(19) United States
(12) Patent Application Publication
(10) Pub. No.: US 2026/0053861 A1
(43) Pub. Date: Feb. 26, 2026

(54) CELL COMPOSITIONS AND METHODS OF USING THE SAME

(71) Applicant: THE REGENTS OF THE UNIVERSITY OF CALIFORNIA, Oakland, CA (US)

(72) Inventors: Faranak FATTAHI, Oakland, CA (US); Homa MAJD, San Francisco, CA (US)

(73) Assignee: THE REGENTS OF THE UNIVERSITY OF CALIFORNIA, Oakland, CA (US)

(21) Appl. No.: 19/104,308

(22) PCT Filed: Aug. 16, 2023
(86) PCT No.: PCT/US2023/072323

§ 371 Date: Feb. 17, 2025

Related U.S. Application Data
(60) Provisional application No. 63/398,470, filed on Aug. 16, 2022.

(51) Int. Cl. A61K 35/30; A61P 25/02; C12N 5/079

(52) U.S. Cl. CPC A61K 35/30; A61P 25/02; C12N 5/0622; C12N 2501/115; C12N 2502/02; C12N 2506/03; C12N 2513/00; C12N 2533/52

(57) ABSTRACT

The present disclosure relates generally to methods and systems of producing Schwann cells from pluripotent stem cells under fully defined conditions. The Schwann cells produced by the disclosed methods find applications as models of the enteric nervous system, tools for high throughput screening of potential therapeutics for treatment of enteric neuropathies, and in regenerative medicine.

### CROSS-REFERENCE TO RELATED APPLICATIONS

This application claims the benefit of U.S. Application No. 63/398,470, filed on Aug. 16, 2022, the contents of which are hereby incorporated by reference in their entireties.

### STATEMENT REGARDING FEDERALLY FUNDED RESEARCH

This invention was made with government support under grant numbers DP2NS116769 and R01DK121169 awarded by the National Institute of Health (NIH). The government has certain rights in the invention.

### SEQUENCE LISTING

The contents of the electronic sequence listing attached herewith (UCAL-031-PCT_Seq List.xml; size: 158,489 bytes; and date of creation: Aug. 15, 2023) is herein incorporated by reference in its entirety.

### TECHNOLOGY FIELD

The present disclosure relates generally to methods of culturing pluripotent stem cells in defined conditions, inducing the pluripotent stem cells to differentiate into Schwann cells. Composition comprising Schwann cells are suitable for screening for potential therapeutic agents of Charcot-Marie-Tooth disease, Schwannomatosis, Guillain-Barre Syndrome and diabetic peripheral neuropathy (DPN) in vitro, and, independently, for applications in regenerative medicine through direct administration or transplantation.

### BACKGROUND

Schwann cells (SCs) are vital components and the major glial cells of the peripheral nervous system (PNS). They are crucial for the development, structural maintenance and function of the nerves and exhibit a remarkable ability to promote neural repair following injury (Jessen and Mirsky, 2005; Lavdas et al., 2008). SCs support axons by forming insulating myelin sheaths and Remak bundles, and provide essential neurotrophic factors. Schwann cells develop from the neural crest (NC) via a Schwann cell precursor (SCP) intermediate that is highly proliferative and migratory. SCPs further differentiate into immature SCs that ultimately give rise to mature myelinating or non-myelinating SCs. In addition to SCs, SCPs can give rise to other derivatives (SCPDs) such as melanocytes (Adameyko et al., 2009; Bonnamour et al., 2021; Nitzan et al., 2013). SC defects are involved in genetic and acquired PNS disorders such as Charcot-Marie-Tooth disease, Schwannomatosis, Guillain-Barre Syndrome and diabetic peripheral neuropathy (DPN) for which there are currently no faithful disease models or effective therapies.

Understanding the development and function of SCs and their roles in PNS health and disease has broad basic and translational implications. However, access to authentic models of human SCs at large scale is a major challenge.

### SUMMARY

The disclosure relates to a method of differentiating at least one or a plurality of stem cells into Schwann cells. The disclosure relates to a composition comprising one or a plurality of Schwann cells, wherein the Schwann cell comprises CD98 or a functional fragment thereof that comprises at least about 70% sequence to CD98. In some embodiments, the cell is derived from a neural crest (NC) cell. In some embodiments, the cell is in culture no fewer than about 35 days. In some embodiments, the cell is in culture no fewer than about 58 days.

In some embodiments, the composition further comprises one or a combination of S100, myelin binding protein (MBP), and GFAP.

In some embodiments, the cell further comprises one or a combination of: SOX10, POU3F2, GAP43, or a functional fragment thereof that comprises at least about 70% sequence identity to SOX10, POU3F2, and GAP43. In some embodiments, the cell comprises an mRNA transcript that encodes one or a combination of: SOX10, POU3F2, GAP43, or a functional fragment thereof that comprises at least about 70% sequence identity to SOX10, POU3F2, and GAP43, respectively.

In some embodiments, the cell further comprises one or a combination of PMP22, SOX10, POU3F2, GAP43, NGFR, MP2, CD46, CD146, CD147, CD166, ERBB3, GDNF, or a functional fragment thereof, such functional fragment of any of one or combination that comprises at least about 70% sequence identity to PMP22, SOX10, POU3F2, GAP43, NGFR, MP2, CD46, CD146, CD147, CD166, ERBB3, and GDNF. In some embodiments, the cell comprises an mRNA transcript that encodes one or a combination of: PMP22, SOX10, POU3F2, GAP43, NGFR, MP2, CD46, CD146, CD147, CD166, ERBB3, GDNF or a functional fragment thereof.

In some embodiments, the cell further comprises one or a combination of FOX01, TBX19, MATN2, PLAT, or a functional fragment thereof that comprises at least about 70% sequence identity to FOX01, TBX19, MATN2, and PLAT. In some embodiments, the cell comprises an mRNA transcript that encodes one or a combination of: FOX01, TBX19, MATN2, PLAT or a functional fragment thereof.

In some embodiments, the cell further comprises one or a combination of PMP22, POU3F2, GAP43, NGFR, MP2, CD46, CD146, CD147, CD166, ERBB3, GDNF, CD9, CD49e, CD171, or a functional fragment thereof that comprises at least about 70% sequence identity to one of PMP22, POU3F2, GAP43, NGFR, MP2, CD46, CD146, CD147, CD166, ERBB3, GDNF, CD9, CD49e, and CD171. In some embodiments, the cell comprises an mRNA transcript that encodes one or a combination of: PMP22, POU3F2, GAP43, NGFR, MP2, CD46, CD146, CD147, CD166, ERBB3, GDNF, CD9, CD49e, CD171 or a functional fragment thereof.

In some embodiments, the Schwann cell comprises MPZ, MAG, PMPP22, PLLP or a functional fragment thereof that comprises at least about 70% sequence to MPZ, MAG, PMPP22, PLLP. In some embodiments, the cell comprises an mRNA transcript that encodes one or a combination of: MPZ, MAG, PMPP22, PLLP or a functional fragment thereof.

In some embodiments, the Schwann cell comprises POU6F2, CD44, CD81 or a functional fragment thereof that comprises at least about 70% sequence to POU6F2, CD44, CD81. In some embodiments, the cell comprises an mRNA transcript that encodes one or a combination of: POU6F2, CD44, CD81 or a functional fragment thereof.

The disclosure relates to a cell line or composition comprising any of the cells identified above. In some embodiments, the disclosure relates to a pharmaceutical composition comprising any of the disclosed cells and a pharmaceutically acceptable carrier. In some embodiments, the compositions disclosed herein comprise greater than about 70% Schwann cells. In some embodiments, the compositions disclosed herein comprise greater than about 80% Schwann cells. In some embodiments, the compositions disclosed herein comprise greater than about 90% Schwann cells. In some embodiments, the cells are derived from human pluripotent stem cells. In some embodiments, the cells are in culture at least about 2 weeks.

The disclosure also relates to a system comprising any of the disclosed cells and a tissue culture medium. In some embodiments, the system further comprises a solid substrate, such as plastic, on which the cells adhere. In some embodiments, the cells are in culture for more than about 2 weeks. In some embodiments, the disclosure relates to an animal or patient comprising any one or plurality of the cells disclosed herein. The disclosure also relates to a tissue culture system comprising a composition of cells as described in the present disclosure and a tissue culture medium. In some embodiments, the system further comprises a solid substrate upon which the cells are positioned.

The disclosure also relates to a pharmaceutical composition comprising a pharmaceutically effective amount of cells as described in the present disclosure; and a pharmaceutically acceptable carrier or excipient.

The disclosure also relates to a method of differentiating a pluripotent stem cell into a Schwann cell, the method comprising exposing an effective amount of FGF2, or a functional fragment thereof, to a neural crest cell for a time period sufficient to differentiate the neural crest into a Schwann cell. The disclosure also relates to a method of enriching Schwann cells in a cell culture comprising exposing a composition of pluripotent stem cells to FGF2, or a functional fragment thereof, for a time period sufficient for the pluripotent stem cell to become a neural crest cell and subsequently exposing the neural crest cell to express to FGF2, or a functional fragment thereof, for a time period sufficient for the neural crest cell to express one or plurality of mRNA encoding SOX10 or a functional fragment thereof. In some embodiments, the neural crest cell expresses an amino acid sequence comprising SOX10 or a functional fragment thereof.

In some embodiments, the method further comprises exposing the composition of neural crest cells with a WNT pathway activator for a time period sufficient for the neural crest cell to express SOX10 or a functional fragment thereof.

In some embodiments, the method further comprises exposing the composition of neural crest cells with SB431542 and/or dbcAMP for a time period sufficient for the neural crest cell to express one or combination of mRNAs encoding or an amino acid comprising: POU3F1, PMP22, MBP, MPZ, AQP4, or a functional fragment thereof. In some embodiments, the step of exposing the composition of neural crest cells with SB431542 and/or dbcAMP comprises exposure for a time period sufficient to differentiate the neural crest cell into a Schwann cell. In some embodiments, the one or plurality of steps of exposing are performed cumulatively for more than about 19 days. In some embodiments, the methods further comprise observing the morphology of the cells and/or performing a polymerase chain reaction (PCR) and/or immunohistochemistry to confirm that differentiation from a neural crest cell to a Schwann cell has occurred. In some embodiments, the cells are in culture in the form of a crestosphere or spheroid.

The disclosure also relates to a method of culturing one or a plurality of Schwann cells comprising exposing one or a plurality of neural crest cells to a tissue culture medium comprising FGF2, SB431542 and/or dbcAMP, or a derivative or functional fragment thereof. In some embodiments, the one or plurality of steps of exposing are performed cumulatively for more than about 19 days. In some embodiments, the method further comprises the step of differentiating a human pluripotent stem cell into a neural crest cell prior to the step of exposing the neural crest cell into a Schwann cell. In some embodiments, the Schwann cells are in culture for no less than about 20, 30, 40, 50, 60, 70, 80, 90, or about 100 days.

The disclosure also relates to a method for screening one or more agents for neuromodulatory activity comprising i) culturing the composition of any one of the disclosed cells in a tissue culture system comprising one or a plurality of healthy or dysfunctional neural cells; ii) exposing the composition to one or more agents; iii) monitoring the composition for neuromodulatory activity; iv) and identifying the one more agents as toxic to healthy cells of the nervous system if the neuromodulatory activity of the agent inhibits or disrupts or reduces viability of the neural cells, as compared to the neuromodulatory activity of the neural cells in the absence of the one or more agents; or identifying the one more agents as inducing repair of neural cells if the neuromodulatory activity of dysfunctional neural cells improves or becomes restored in respect to function as compared to the neuromodulatory activity of dysfunctional neural cells in the absence of the one or more agents.

The disclosure also relates to a method of transplanting a Schwann cell population into a subject in need thereof by administering to the subject a pharmaceutical composition comprising a therapeutically effective amount of any one or combination of disclosed Schwann cells and a pharmaceutically acceptable carrier.

The disclosure also relates to a method of treating a spinal cord injury in a subject in need thereof comprising administering to the subject a therapeutically effective amount of an agent or a pharmaceutical composition comprising a therapeutically effective amount of any one or combination of disclosed Schwann cells and a pharmaceutically acceptable carrier.

The disclosure also relates to a method of treating diabetic peripheral neuropathy in a subject in need thereof comprising administering to the subject a therapeutically effective amount of an agent or a pharmaceutical composition comprising a therapeutically effective amount of any one or combination of disclosed Schwann cells and a pharmaceutically acceptable carrier. In some embodiments, the agent is chosen from an agent of Table S4. In some embodiments, the agent is chosen from an agent of Table S4 or a pharmaceutically acceptable salt or derivative thereof. In some embodiments, the agent is chosen from an agent of Table S4, or a pharmaceutically acceptable salt or derivative thereof, that comprises a Z score above 1.0, 1.1, 1.2, 1.3, 1.4, 1.5, 1.6, 1.7, 1.8, 1.9, or above 2.0.

### BRIEF DESCRIPTION OF THE DRAWINGS

FIGS. 1A through 1J: Deriving Schwann cells from hPSCs. FIG. 1A. Schematic illustration of the protocol for deriving developing precursors and Schwann cell (SC) cultures from hPSCs-derived neural crests (NCs). FIG. 1B. SOX10::GFP expression at day 11, 25 and 35 of differentiation. Scale bars=100 μm in B left and middle panel and 25 μm in B right panel. FIG. 1C. Representative immunofluorescence images of hPSC-derived SCs for Schwann lineage markers at day 60. Scale bar=25 pam. FIG. 1D. Quantification of markers in (D). FIG. 1E. UMAP visualization of scRNA-seq data for low passage (Day 38) and high passage (Day 58) SC cultures. SCPD: Schwann cell precursor derived; SCP: Schwann cell precursor; SC: Schwann cells. FIG. 1F. Dot plot of the scaled average expression of SC differentiation and myelination (left) and nerve support (right) markers in single cell RNA-seq data of low and high passage Schwann cells. FIG. 1G. Dot plot of the scaled average expression of top 15 primary mouse myelinating (mySC) and non-myelinating (nmSC) Schwann cell markers in single cell RNA-seq data of low and high passage Schwann cell culture. FIG. 1H. Module scoring of top 100 high passage (HP) Schwann cell type specific DE marker genes in low passage (LP) Schwann cell types (left). Feature (left) and dot plot (right) visualizations are depicted. FIG. 1I. Module scoring top 100 low passage (LP) Schwann cell type specific DE marker genes in high passage (HP) Schwann cell types (left). Feature (left) and dot plot (right) visualizations are depicted. FIG. 1J. Principal component analysis (PCA) of NC cells, developing precursors, human primary Schwann cells, and hPSC-derived SC cultures at day 50 and day 100 of differentiation in comparison with central nervous system (CNS) precursors.

FIGS. 2A through 2J: hPSC-derived Schwann cells myelinate hPSC-derived sensory neurons and engraft in injured rat sciatic nerves. FIG. 2A. Feature plots of mature SC clusters isolated from low (top) and high (bottom) passage culture single cell RNA-seq data with dark colors indicating SCs identified as myelinating (mySC). Bar plots show the relative population of mySCs. FIG. 2B. Pathway enrichment analysis of top 250 DE genes of myelinating mature SCs in low (left) and high (right) passage cells. Top 50 pathways from combined GO BP, Reactome and KEGG analysis are shown. FIG. 2C. Schematic illustration of the hPSC-SC co-cultures with hPSC-derived sensory or motor neurons. FIG. 2D. Physical association of hPSC-SCs with hPSC-sensory neurons. FIG. 2E. Physical association of hPSC-SCs with hPSC-derived motor neurons. FIG. 2F. Schematic illustration of hPSC-SC transplantation in adult rat sciatic nerves. RFP+ hPSC-derived Schwann cells were injected following nerve crush at the site of injury (adult Cyclosporin-A treated SD rats). FIG. 2G. Immunofluorescence staining of grafted sciatic nerves for human specific nuclear marker SC101 at 8 weeks post transplantation. FIG. 2H. Confocal analysis of teased sciatic nerve fibers for RFP (grafted human cells), axonal marker (NFH) and DAPI. FIG. 2I. Confocal analysis of teased nerve fibers for RFP (grafted human cells), myelin markers MAG and P0 and DAPL FIG. 2J-2L. Confocal analysis of teased nerve fibers for RFP and node markers Pan-Na+ (sodium channel, arrow heads, FIG. 2H), CASPR (arrow heads, FIG. 2I) and Kv1.2 (K+ channel, arrow heads, FIG. 2J). Scale bars=100 μm in FIG. 2B left panel, 20 μm in B right panel, 0.2 μm in FIG. 2C, 100 μm in, 20 μm in FIG. 2F and FIG. 2G and 10 μm in FIG. 2H-2J.

FIGS. 3A through 3P: Schwann cells are selectively vulnerable to high glucose exposure. FIG. 3A. Schematic illustration of the experimental paradigm for modeling diabetic nerve damage in hPSC-derived cell types. FIG. 3B. Lactate dehydrogenase (LDH) cytotoxicity analysis of hPSC-derived SCs and sensory neurons in response to exposure to different glucose concentration using LDH activity assay. FIG. 3C. Oxidative stress measurement of hPSC-derived SCs exposed to increasing concentration of glucose. Statistical analysis was performed using one-way ANOVA comparing values to the low glucose (5 mM) condition. ns, not significant, p-values are: *p<0.05; **p<0.01. FIG. 3D. Schematic illustration of high-throughput drug screening for identification of compounds that enhance the viability of high glucose-treated hPSC-SCs. FIG. 3E. Presentation of the distribution of library compounds by their corresponding normalized viability z-score. FIG. 3F. Gene-set enrichment analysis using iPAGE for the library compounds targets identifies GO terms associated with hits improving and worsening SC viability. FIG. 3G. p-value correlation plot to identify the genes that are most likely the targets of the effective treatment. Normalized z-score from all the treatments associated with a gene are integrated. In addition, to assess the enrichment of individual genes among those that are targets of the treatments with increased z-scores, a Fisher's exact test is performed. The plot shows the correlation between the p-values. FIG. 3H. One-sided volcano plot showing the average z-score vs. −log of p-value for all genes with positive z-scores. The genes that pass the statistical thresholds of combined z-score FDR<0.25 and Fisher's p-value <0.1 are marked in gold. FIG. I. Identified target genes (marked gold in H) ranked by their combined z-score. FIG. 3J. Protein-protein interaction network of the identified target genes (listed in I) constructed by STRING database. Minimum required interaction score was set to 0.4 and edge thickness indicates the degree of data support. FIG. 3K. Predicted targets of hits in (K) compiled from the following databases: BindingDB (Liu et al., 2007), Carlsbad (Mathias et al., 2013), Dinies (Yamanishi et al., 2014), PubChem bioassays (Kim et al., 2019), SEA (Keiser et al., 2007), Superdrug 2 (Siramshetty et al., 2018) and SwisTargetPrediction (Gfeller et al., 2014). FIG. 3L. Schematic illustration of the unbiased metabolite and transcriptional profiling of differentially treated SCs. FIG. 3M. Pathway enrichment analysis of genes upregulated in high glucose and downregulated upon BP treatment. FIG. 3N. Glycerolipid metabolism enzymes upregulated in high glucose condition. FIG. 3O. Glycerolipid metabolism schematic adopted from KEGG shows changes in the enzymes and metabolites in response to high glucose and BP treatments in SCs. HighGluc: high glucose, LowGluc: low glucose, HighGluc.Bup: high glucose plus bupropion, LowGluc.Bup: low glucose plus bupropion. FIG. 3P. PTGER4 KO in SCs rescues high glucose treated SCs. CRISPR-Cas9 mediated knocking out of PTGER4 in SCs protects them against increased levels of cleaved caspase 3 (marker of apoptosis) under high glucose condition as measured by flow cytometry. p-values are: *p<0.05; **p<0.01; ***p<0.001.

FIG. 4A through 4F: Bupropion treatment prevents diabetic nerve damage in mice. FIG. 4A. Schematic illustration of modeling diabetes and Bupropion treatment in mice. FIG. 4B. Thermal sensitivity test measuring the latency of hind paw withdrawal in normal mice and mice treated with STZ and Bupropion. FIGS. 4C and 4D. TUNEL staining (FIG. 4C) and quantification (FIG. 4D) in sciatic nerves of normal mice and mice treated with STZ and Bupropion. FIGS. 4E and 4F. Electron transmission microscopy (FIG. 4E) and quantification (FIG. 4F) of damaged myelin structures in sciatic nerves of normal mice and mice treated with STZ and Bupropion. p-values are: *p<0.05; **p<0.01; ***p<0.001; ****p<0.0001. Scale bars=100 μm in FIG. 4C and 5 μm in FIG. 4E. BP, Bupropion HCL.

FIGS. 5A through 5B: Characterization of hPSC-derived SC lineages. FIG. 5A. qRT-PCR for a panel of Schwann cell and precursor markers at different timepoints of the differentiation protocol. Log 2 fold change relative to D0 of differentiation is shown. FIG. 5B. qRT-PCR for a panel of Schwann lineage markers involved in Schwann cell differentiation and myelination (left) and nerve interaction and support (right).

FIGS. 6A through 6D: Sub-type specific characterization of hPSC-derived SC lineages. FIG. 6A. Dot plot of the scaled average expression of the top ten differentially expressed (DE) genes for each low and high passage Schwann cell types. SCPD: Schwann cell precursor derived; SCP: Schwann cell precursor; SC: Schwann cells. FIGS. 6B-6D. Dot plots of scaled average expression of low and high passage cell type specific neurotrophic factors (FIG. 6B), neurotransmitter receptors and postsynaptic signal transmission (FIG. 6C), and transcription factors (FIG. 6D). All gene sets were prior filtered to include genes expressed in at least 25% of cells in either cell type in low (LP) and high (HP) passage SC culture data.

FIG. 7A through 7H: Molecular changes in SC cultures after long-term maintenance. FIGS. 7A-7B. Feature plot (left) and distribution of cell cycle phases (right) in low passage (FIG. 7A) and high passage (FIG. 7B) Schwann cell cultures. FIG. 7C UMAP visualization of merged low and high passage Schwann cell dataset. FIG. 7D Feature plots showing module scoring of top 100 low passage (top) and high passage (bottom) cell type specific DE marker genes in the merged dataset. FIGS. 7E-7G UMAP visualization, (FIG. 7E) distribution of cell cycle phases (FIG. 7F), and cell population proportion (FIG. 7G) in low and high passage merged dataset depicting subclusters of early SCs and SCPDs. FIG. 7H. Gene ontology biological process (GO BP) pathway enrichment analysis on top 250 differentially expressed genes in low and high passage SCPDs.

FIGS. 8A through 8D: Antibody screen identifies novel surface markers for human SCs. FIG. 8A. Schematic illustration of the human antibody screening paradigm. FIG. 8B. Primary screening identifies novel surface markers for hPSC-SCs. FIG. 8C. Dot plot of the scaled average expression of low and high passage cell type specific surface markers. Surface markers identified in the human antibody screen highlighted in red. Gene set was prior filtered to include genes expressed in at least 25% of cells in either cell type in low (LP) and high (HP) passage SC culture data. FIG. 8D. Immunocytochemistry (left), flow cytometry-based (right) validation of surface marker expression at different stages of SC differentiation.

FIG. 9A through 9C: Molecular characterization of myelinating SCs. FIGS. 9A-9C. Dot plots of scaled average expression of specific neurotrophic factors (FIG. 9A), neurotransmitter receptors (FIG. 9B), and Cell Adhesion Molecules (FIG. 9C) in myelinating SCs (myScs) and other cells in low and high passage cultures. All gene sets were prior filtered to include genes expressed in at least 25% of cells in either cell type in low (LP) and high (HP) passage SC culture data.

FIG. 10A through 10B. hPSC-SCs accelerate functional maturation of hPSC-motor neurons in co-cultures. FIGS. 10A-10B. Calcium imaging of motor neuron single culture and motor-neuron Schwann co-cultures at day 40 (FIG. 10A) and 70 (FIG. 10B) of motor neuron differentiation.

FIG. 11. hPSC-SCs expression profile in connection with differentiation factors. Expression plots of various expression patterns of genes related to CMT in cells that have different degrees of maturation.

FIG. 12. Bupropion treatment is associated with lower odds of neuropathy in diabetic patients (Left and Middle Panels) Schematic (Left Panel) and Venn diagram (Middle Panel) of the cohort of diabetes individuals derived from health records. (Right Panel) Association of bupropion with neuropathy in diabetic patients in multivariate logistic models adjusted for age, duration of diabetes, sex, smoking, and antidepressant drug treatment.

FIGS. 13A through 13D: Association of BP and other drugs with diabetic neuropathy Schematic (FIG. 13A) and Venn diagram (FIG. 13B) of the cohort of diabetic individuals filtered for age (older than 40 years) and duration of diabetes (longer than 10 years) in the University of California San Francisco (UCSF) de-identified health records. Incidence of neuropathy is evaluated in individuals taking gabapentin & pregabalin (green), bupropion (blue), fluoxetine (an SSRI) (light red) and armitriptyline (a TCA) (orange). Neuropathy is highlighted. FIG. 13C. Association of neuropathy with bupropion, fluoxetine, gabapentin & pregabalin and amitriptyline in diabetic individuals in multivariate logistic models adjusted for age, duration of diabetes, sex, smoking. FIG. 13D. Anti-depressive and anti-psychotic drugs in the FDA approved drug library, ordered according to their z-scores for effect on SC viability when co-treated with 30 mM glucose.

### DETAILED DESCRIPTION OF EMBODIMENTS

The disclosed methods and compositions may be understood more readily by reference to the following detailed description of particular embodiments and the examples included therein and to the figures and their previous and following description. It is to be understood that the disclosed methods and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

The disclosure relates to hPSC differentiation strategies (methods of differentiating cells) for efficient derivation of SCs that recapitulate molecular features and function of primary SCs. We characterized the diversity of cell types in our cultures using a combination of imaging and high-resolution transcriptomic profiling and identified novel markers and molecular signatures for human SC subtypes. We further validated the engraftment potential of these cells upon transplantation into a rat model of peripheral neuropathy. Finally, we leveraged hPSC-derived SCs to model the most common cause of peripheral neuropathy, i.e. diabetic peripheral neuropathy (DPN) that affects 30-60% of diabetic patients (Callaghan et al., 2012) and is the leading cause of diabetes-related hospital admissions and nontraumatic lower-extremity amputations (Boulton et al., 2005). The pathogenesis of DPN is complex involving vascular disease, hyperglycemia, hypoxia and oxidative stress that result in cytotoxicity and progressive degeneration of peripheral nerves (Simmons and Feldman, 2002). While symptoms arise from neuronal dysfunction, it is unclear whether sensory neuron damage is the primary event in DPN, and there is evidence that SC degeneration and peripheral demyelination may be contributing factors (Eckersley, 2002). Dissecting cell type specific mechanisms is challenging using current animal models of DPN given the involvement of various non-cell autonomous factors including systemic vascular abnormalities. We utilized hPSC-derived SCs and sensory neurons to determine cell type specific vulnerabilities to high glucose, establish an alternative human-based model of DPN and identify potential therapeutic candidates. Therefore, there remains a need for novel protocols for derivation of enteric neurons (ENs) from hPSCs and a basis for modeling ENS development and the contribution of specific lineages to ENS disease.

The disclosure relates to compositions comprising SCs. The disclosure further relates to a tissue culture system. The disclosure also relates to methods of differentiating pluripotent stem cells into SCs, methods of enriching SCs in a cell culture system, and methods of culturing SCs. It should be appreciated that such methods find applications, for example, in probing the genetic contributions underpinning ENS pathogenesis using induced pluripotent stem cell (iPSC) lines. In some embodiments the cells are from stem cells from patients suffering from enteric neuropathies. Disease phenotypes can be modeled through in vitro differentiations and addressed via genetic or molecular perturbation strategies. As such, the disclosure also relates to methods for screening compounds, methods for transplanting SCs, and methods for treating a spinal cord injury and peripheral nerve damage by administering pharmaceutical compositions comprising any one or combination of disclosed cells and a pharmaceutically acceptable carrier.

Unless defined otherwise, technical and scientific terms used herein have the same meaning as commonly understood by one of ordinary skill in the art to which this invention belongs. For example, Singleton et al., Dictionary of Microbiology and Molecular Biology 2nd ed., J. Wiley & Sons (New York, NY 1994), provide one skilled in the art with a general guide to many of the terms used in the present application. Additionally, the practice of the present invention will employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual”, 2nd edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4th edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); and “PCR: The Polymerase Chain Reaction”, (Mullis et al., eds., 1994).

As used in the present disclosure and claims, the singular forms “a”, “an” and “the” include plural forms unless the context clearly dictates otherwise. Thus, for example, reference to “a nucleic acid sequence” includes a plurality of nucleotides that are present, and reference to “the nucleic acid sequence” is a reference to one or more nucleic acid sequences and equivalents thereof known to those skilled in the art, and so forth.

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “substantially free of” as used herein refers to a composition that only has trace or negligible amounts of the substance to which it refers. In some embodiments, substantially free means that the composition comprises only about 0.1%, 0.2%, 0.3% 0.4% or 0.5% of the substance to which it refers. In some embodiments, substantially free means that the composition comprises less than about 1.0% of the substance to which it refers relative to the number or mass of substances in the compositions and confers no biological effect to the compositions.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. The term “about” as used herein when referring to a measurable value such as an amount, a temporal duration, and the like, is meant to encompass variations of ±20%, ±10%, ±5%, ±1%, ±0.5%, or ±0.1% from the specified value, as such variations are appropriate to perform the disclosed methods.

“Contacting” is used in accordance with its plain ordinary meaning and refers to the process of allowing at least two distinct species (e.g., chemical compounds, including biomolecules, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a cell (e.g., a Schwann cell). In some embodiments contacting includes allowing a compound described herein to interact with a protein or enzyme that is involved in a signaling pathway, such as PDGFR.

The term “culture vessel” as used herein is defined as any vessel suitable for growing, culturing, cultivating, proliferating, propagating, or otherwise similarly manipulating cells. A culture vessel may also be referred to herein as a “culture insert”. In some embodiments, the culture vessel is made out of biocompatible plastic and/or glass. In some embodiments, the plastic is a thin layer of plastic comprising one or a plurality of pores that allow diffusion of protein, nucleic acid, nutrients (such as heavy metals and hormones) antibiotics, and other cell culture medium components through the pores. In some embodiments, the pores are not more than about 0.1, 0.5 1.0, 2, 3, 4, 5, 6, 7, 8, 9, 10, 15, 20, 25, 30, 40, 50 microns wide. In some embodiments, the culture vessel in a hydrogel matrix and free of a base or any other structure. In some embodiments, the culture vessel is designed to contain a hydrogel or hydrogel matrix and various culture mediums. In some embodiments, the culture vessel consists of or consists essentially of a hydrogel or hydrogel matrix. In some embodiments, the only plastic component of the culture vessel is the components of the culture vessel that make up the side walls and/or bottom of the culture vessel that separate the volume of a well or zone of cellular growth from a point exterior to the culture vessel. In some embodiments, the culture vessel comprises a hydrogel and one or a plurality of isolated Schwann cells. In some embodiments, the culture vessel comprises a hydrogel and one or a plurality of isolated pluripotent stem cells or, to which one or a plurality of neuronal cells are seeded.

The term “exposing” as used herein refers to bringing a disclosed compound and a cell, target receptor, or other biological entity together in direct or indirect contact, in such a manner that the compound can affect the activity of the cell (e.g., receptor, cell, etc.). Directly this can occur by physical contact between the disclosed compound and the cell, receptor o other entity; i.e., by interacting with the target or cell itself, or indirectly this can occur by interacting with another molecule, co-factor, factor, or protein on which the activity of the cell is dependent. In some embodiments, the activity of the cell in response to the compound or molecule is differentiation. In some embodiments, the compound is one or more differentiation factors.

“Analogues” or “derivatives,” as used interchangeably, of the compounds disclosed herein are pharmaceutically acceptable salts, prodrugs, deuterated forms, radio-actively labeled forms, isomers, solvates and combinations thereof. The “combinations” mentioned in this context are refer to derivatives falling within at least two of the groups: pharmaceutically acceptable salts, prodrugs, deuterated forms, radio-actively labeled forms, isomers, and solvates. Examples of radio-actively labeled forms include compounds labeled with tritium, phosphorous-32, iodine-129, carbon-11, fluorine-18, and the like. The compounds described herein may be present in the form of pharmaceutically acceptable salts. For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Examples of pharmaceutically acceptable base addition salts include e.g., sodium, potassium, calcium, ammonium, organic amino, or magnesium salt.

As used herein, the term “salt” refers to acid or base salts of the compounds used in the methods of the present disclosure. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The term “pharmaceutically acceptable excipient, carrier or diluent” as used herein is meant to refer to an excipient, carrier or diluent that can be administered to a subject, together with an agent, and which does not destroy the pharmacological activity thereof and is nontoxic when administered in doses sufficient to deliver a therapeutic amount of the agent. The term “pharmaceutically acceptable salt” of nucleic acids as used herein may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, suifanilic, formic, toluenesulfonie, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenyiacetic, a!kanoic such as acetic, HOOC—(CH2)n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts for the pooled viral specific antigens or polynucleotides provided herein, including those listed by Remington's Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

The term “progenitor cell” as used herein is defined as a cell that is pluripotent cell exposed to cell medium that comprises differentiation factors but remains pluripotent at least partially undifferentiated. In some embodiments, a progenitor cell comprises WNT2B+. In some embodiments, a progenitor cell comprises PAX6+.

The term “pluripotent stem cell” as used herein is defined as a cell that is self-replicating and capable of developing into cells and tissues of the three primary germ layers. Pluripotent stem cells include embryonic and induced pluripotent cells as defined herein. Contemplated pluripotent stem cells originate from mammals, e.g., human, mouse, rat, monkey, horse, goat, sheep, dog, cat etc.

The term “induced pluripotent stem cell” (iPSC) means a type of pluripotent cell made by reprogramming a somatic cell to have the same properties as embryonic stem cells, namely, the ability to self-renew and differentiate into the three primary germ layers. In some embodiments, iPSCs include mammalian cells, e.g., human, mouse, rat, monkey, horse, goat, sheep, dog, cat etc., reprogrammed to express Oct4, Nanog, Sox2, and optionally c-Myc. In some embodiments, iPSCs comprise reprogrammed primary cell lines. In some embodiments. iPSCs are obtained from a repository, such as the Coriell Institute for Medical Research (e.g., Catalog ID GM25256 (WTC-11), GM25430, GM23392, GM23396, GM24666, GM27177, GM24683), California Institute for Regenerative Medicine: California's Stem Cell Agency (e.g., CW60261, CW60354, CW60359, CW60480, CW60335, CW60280, CW60594, CW60083, CW60086, CW60087, CW60167, CW60186), and the American Type Culture Collection (ATCC®) (e.g., ATCC-DYR0530 Human Induced Pluripotent Stem (IPS) Cells (ATCC® ACS-1012™, ATCC® ACS-1011™, ATCC® Number: ACS-1024™, ATCC® Number: ACS-1028™, ATCC® Number: ACS-1031™, ATCC® Number: ACS-1004™, ATCC® Number: ACS-1029™, ATCC® Number: ACS-1020™, ATCC® Number: ACS-1007™, ATCC® Number: ACS-1030™) Induced pluripotent stem cells may be derived from cell types such as fibroblasts taken from the skin, lung, or vein of subjects that are apparently healthy or diseased. In some embodiments, iSPCs are isolated from a subject suffering from an indication disclosed herein such as diabetic peripheral neuropathy.

As defined herein, the term “inhibition,” “inhibit,” “inhibiting,” and the like in reference to a protein-inhibitor (e.g., antagonist) interaction means negatively affecting (e.g., decreasing) the activity or function of the protein relative to the activity or function of the protein in the absence of the inhibitor. In embodiments inhibition refers to reduction of a disease or symptoms of disease. In embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein.

The term “embryonic stem cell line” as used herein is defined as a cell derived from the inner cell mass of the pre-implantation blastocyst capable of self-renewal and differentiation into the three primary germ layers. In some embodiments, embryonic stem cell lines listed in the NIH Human Embryonic Stem Cell Registry, e.g., CHB-1, CHB-2, CHB-3, CHB-4, CHB-5, CHB-6, CHB-8, CHB-9, CHB-10, CHB-11, CHB-12, RUES1, RUES2, HUES 1, HUES 2, HUES 3, HUES 4, HUES 5, HUES 6, HUES 7, HUES 8, HUES 9, HUES 10, HUES 11, HUES 12, HUES 13, HUES 14, HUES 15, HUES 16, HUES 17, HUES 18, HUES 19, HUES 20, HUES 21, HUES 22, HUES 23, HUES 24, HUES 26, HUES 27, HUES 28, CyT49, RUES3, WA01 (H1), UCSF4, NYUES1, NYUES2, NYUES3, NYUES4, NYUES5, NYUES6, NYUES7, MFS5, HUES 48, HUES 49, HUES 53, HUES 65, HUES 66, UCLA 1, UCLA 2, UCLA 3, WA07 (H7), WA09 (H9), WA13 (H13), WA14 (H14), HUES 62, HUES 63, HUES 64, CT1, CT2, CT3, CT4, MA135, Endeavour-2, WIBR1, WIBR2, HUES 45, Shef 3, Shef 6, WIBR3, WIBR4, WIBR5, WIBR6, BJNhem19, BJNhem20, SA001, SA002, UCLA 4, UCLA 5, UCLA 6, HUES PGD 13, HUES PGD 3, ESI-014, ESI-017, HUES PGD 11, HUES PGD 12, WA15, WA16, WA17, WA18, WA19, etc. In some embodiments, embryonic stem cells comprise gene(s) associated with diseases or disorders.

The term “enteric neural crest cell” or “neural crest cell” means a cell produced by inducing differentiation of a pluripotent stem cell, wherein the enteric neural crest cell expresses SOX10, PHOX2B, EDNRB, TFAP2A, BRN3A, ISL1 and/or ASCL1. In some embodiments, the enteric neural crest cell comprises FOX3D. In some embodiments, the neural crest cell is present in an embryoid body or neural rosette. In some embodiments, the neural crest cell expresses vagal markers HOXB2, HOXB3, and/or HOXB5. In some embodiments, neural crest cells express p75 and HNK1. In some embodiments, neural crest cells express HOXB2, HOXB3, HAND2 and EDNRB. In some embodiments, the neural crest cell or enteric neural crest is isolated from a primary stem cell or an induced pluripotent stem cell. In some embodiments, the neural crest cells are any of those cells identified as “neural crest cells” in PCT/US2021/024244 or PCT/US2019/068447, both of which are incorporated by reference in their entireties. In some embodiments, the Schwann cells are any of those cells identified as Schwann cells in WO 2018/090002 or WO 2018/090006, both of which are incorporated by reference in their entireties.

The term “enteric neuron” means a cell that exhibits downregulation of SOX10, sustained expression of EDNRB, ASCL1 and PHOX2B, and upregulation of TUJ1 and TRKC. In some embodiments, enteric neurons express neuronal subtype specific markers including the cholinergic neuronal marker Choline Acetyl Transferase (CHAT), serotonin (5-HT) receptor, gamma-Aminobutyric acid (GABA), and neuronal nitric oxide synthase (nNOS). In some embodiments, CHAT expression indicates the presence of cholinergic neurons. In some embodiments, expression of NOS1 indicates the presence of nitrergic neurons. In some embodiments, enteric neurons include glial cells expressing glial fibrillary acidic protein (GFAP) and SOX10. In some embodiments, the enteric neuron is produced by inducing differentiation of an enteric neural crest cell. In some embodiments, the enteric neurons express SOX10, sustained expression of EDNRB, ASCL1 and PHOX2B, and upregulation of TUJ1 and TRKC.

The term “enteric glial cell” means a cell that exhibits expression of SOX10 and: GPAP and/or PMP22. In some embodiments, the enteric glial cell exhibits expression of SOX10 and PMP22. In some embodiments, the enteric glial cells is produced by inducing differentiation of an enteric neural crest cell.

The term “Schwann cell” means a cell that is a neural crest-derived nerve associated cell from the peripheral nervous system. In some embodiments, Schwann cells interact with neurons and support their function if positioned in an animal or patient. In some embodiments, Schwann cells express nucleic acid sequences that encode or amino acid sequences that comprise at least about 90% sequence identity to CD98, SOX10, POU3F2, NGFR and/or GFAP43, or functional fragments thereof. In some embodiments, the cells exhibits expression of one or a combination of the proteins or nucleic acid sequences that encodes a protein identified in any of claims 1 through 10.

The term “rho kinase inhibitor” means a compound that decreases the activity of rho kinase. In some embodiments, the rho kinase inhibitor is N-[(3-Hydroxyphenyl)methyl]-N′-[4-(4-pyridinyl)-2-thiazolyl]urea dihydrochloride (RKI-1447), (+)-(R)-trans-4-(1-aminoethyl)-N-(4-pyridyl)cyclohexanecarboxamide dihydrochloride (Y-27632), Fasudil (HA-1077), Hydroxyfasudil (HA 1100 hydrochloride), Thiazovivin, GSK429286A, Narciclasine, and/or (+)-(R)-trans4-(1-aminoethyl)-N-(1H-pyrrolo[2,3-b]pyridin-4-yl)cyclohexanecarboxamide dihydrochloride (Y-30141).

The term “hydrogel” as used herein is defined as any water-insoluble, crosslinked, three-dimensional network of polymer chains with the voids between polymer chains filled with or capable of being filled with water. The term “hydrogel matrix” as used herein is defined as any three-dimensional hydrogel construct, system, device, or similar structure. In some embodiments, the hydrogel or hydrogel matrix comprises one or more proteins and/or glycoproteins. In some embodiments, the hydrogel or hydrogel matrix comprises one or more of the following proteins: collagen, gelatin, elastin, titin, laminin, fibronectin, fibrin, keratin, silk fibroin, and any derivatives or combinations thereof. In some embodiments, the hydrogel or hydrogel matrix comprises Matrigel® or vitronectin. In some embodiments, the hydrogel or hydrogel matrix can be solidified into various shapes, for example, a bifurcating shape designed to mimic a neuronal tract. In some embodiments, the hydrogel or hydrogel matrix comprises poly (ethylene glycol) dimethacrylate (PEG). In some embodiments, the hydrogel or hydrogel matrix comprises Puramatrix. In some embodiments, the hydrogel or hydrogel matrix comprises glycidyl methacrylate-dextran (MeDex). In some embodiments, two or more hydrogels or hydrogel matrixes are used simultaneously cell culture vessel. In some embodiments, two or more hydrogels or hydrogel matrixes are used simultaneously in the same cell culture vessel but the hydrogels are separated by a wall that create independently addressable microenvironments in the tissue culture vessel such as wells. In a multiplexed tissue culture vessel it is possible for some embodiments to include any number of aforementioned wells or independently addressable location within the cell culture vessel such that a hydrogel matrix in one well or location is different or the same as the hydrogel matrix in another well or location of the cell culture vessel.

The term “Matrigel®” means a solubilized basement membrane preparation extracted from the Engelbreth-Holm-Swarm (EHS) mouse sarcoma comprising ECM proteins including laminin, collagen IV, heparin sulfate proteoglycans, entactin/nidogen, and other growth factors. In some embodiments, Cultrex® BME (Trevigen, Inc.) or Geltrex® (Thermo-Fisher Inc.) may be substituted for Matrigel®.

The term “two-dimensional culture” as used herein is defined as cultures of cells on flat hydrogels, including Matrigel® and vitronectin, disposed in culture vessels.

As used herein, a “spheroid” or “cell spheroid” means any grouping of cells in a three-dimensional shape that generally corresponds to an oval or circle rotated about one of its principal axes, major or minor, and includes three-dimensional egg shapes, oblate and prolate spheroids, spheres, and substantially equivalent shapes.

The term “subject” as used herein refers to any animal (e.g., a mammal), including, but not limited to, humans, non-human primates, canines, felines, rodents, and the like. In some embodiments, the subject is a human subject. The terms “subject,” “individual,” and “patient” are used interchangeably herein. The terms “subject,” “individual,” and “patient” thus encompass individuals having cancer (e.g., breast cancer), including those who have undergone or are candidates for resection (surgery) to remove cancerous tissue.

As used herein, the term “diagnosed” means having been subjected to a physical examination by a person of skill, for example, a physician, and found to have a condition that can be diagnosed or treated by the compounds, compositions, or methods disclosed herein. In some embodiments of the disclosed methods, the subject has been diagnosed with a need for treatment of a nerve damage or DNP, prior to the administering step. As used herein, the phrase “identified to be in need of treatment for a disorder,” or the like, refers to selection of a subject based upon need for treatment of the disorder. It is contemplated that the identification can, in some embodiments, be performed by a person different from the person making the diagnosis. It is also contemplated, in further embodiments, that the administration can be performed by one who subsequently performed the administration.

The term “associated” or “associated with” in the context of a substance or substance activity or function associated with a disease (e.g., a protein associated disease, a symptom associated with nerve repair or dysfunction, a symptom associated with the disease (e.g., diabetic peripheral neuropathy) is caused by (in whole or in part), or a symptom of the disease is caused by (in whole or in part) the substance or substance activity or function. For example, a symptom of a condition may be a symptom that results (entirely or partially) from spinal cord damage or DNP. As used herein, what is described as being associated with a disease, if a causative agent, could be a target for treatment of the disease.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a device comprising disclosed cells, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., cardiomyopathy therapies including, for example, Angiotensin Converting Enzyme Inhibitors (e.g., Enalipril, Lisinopril), Angiotensin Receptor Blockers (e.g., Losartan, Valsartan), Beta Blockers (e.g., Lopressor, Toprol-XL), Digoxin, or Diuretics (e.g., Lasix; or Parkinson's disease therapies including, for example, levodopa, dopamine agonists (e.g., bromocriptine, pergolide, pramipexole, ropinirole, piribedil, cabergoline, apomorphine, lisuride), MAO-B inhibitors (e.g., selegiline or rasagiline), amantadine, anticholinergics, antipsychotics (e.g., clozapine), cholinesterase inhibitors, modafinil, or non-steroidal anti-inflammatory drugs.

As used herein, the terms “subject,” “individual,” “host,” and “patient,” are used interchangeably herein and refer to a vertebrate animal, including but not limited to a mammal or human, for whom diagnosis, treatment or therapy is desired, particularly humans. Mammals include, but are not limited to, murines, simians, humans, farm animals, cows, pigs, goats, sheep, horses, dogs, sport animals, rats and pets. Tissues, cells and their progeny obtained in vivo or cultured in vitro are also encompassed by the definition of the term “subject.” The methods described herein are applicable to both human therapy and veterinary applications. In some instances in the description of the present disclosure, the term “patient” refers to human patients suffering from a particular disease or disorder. In some embodiments, the subject may be a human suspected of having or being identified as at risk to develop a peripheral neuropathy. In some embodiments, the subject may be diagnosed as having DPN and of having or being identified as at risk to develop DPN. In some embodiments, the subject is a mammal, and, in other embodiments, the subject is a human. In some embodiments, the subject is a non-human vertebrate.

As used herein, the term “therapeutic” means an agent utilized to treat, combat, ameliorate, prevent or improve an unwanted condition or disease of a patient.

A “therapeutically effective amount” or “effective amount” of a composition is a predetermined amount calculated to achieve the desired effect, i.e., to treat, combat, ameliorate, prevent or improve one or more symptoms of a viral infection. The activity contemplated by the present methods includes both medical therapeutic and/or prophylactic treatment, as appropriate. The specific dose of a compound administered according to the present disclosure to obtain therapeutic and/or prophylactic effects will, of course, be determined by the particular circumstances surrounding the case, including, for example, the compound administered, the route of administration, and the condition being treated. It will be understood that the effective amount administered will be determined by the physician in the light of the relevant circumstances including the condition to be treated, the choice of compound to be administered, and the chosen route of administration, and therefore the above dosage ranges are not intended to limit the scope of the present disclosure in any way. A therapeutically effective amount of compounds of embodiments of the present disclosure is typically an amount such that when it is administered in a physiologically tolerable excipient composition, it is sufficient to achieve an effective systemic concentration or local concentration in the tissue. The terms “treating” or “treatment” or “treat” as used herein refer to therapeutic measures that cure, slow down, lessen symptoms of, and/or halt progression of a diagnosed pathologic condition or disorder.

The term “preventing” or “prevention” or “prevent” as used herein refers to prophylactic or preventative measures that prevent or slow the development of a targeted pathologic condition or disorder. Those in need of treatment include those already diagnosed with the disorder; those prone to have the disorder; and those in whom the disorder is to be prevented.

The “percent identity” or “percent homology” of two polynucleotide or two polypeptide sequences is determined by comparing the sequences using the GAP computer program (a part of the GCG Wisconsin Package, version 10.3 (Accelrys, San Diego, Calif.)) using its default parameters. “Identical” or “identity” as used herein in the context of two or more nucleic acids or amino acid sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0. Briefly, the BLAST algorithm, which stands for Basic Local Alignment Search Tool is suitable for determining sequence similarity. Software for performing BLAST analyses is publicly available through the National Center for Biotechnology Information (http://www.ncbi.nlm.nih.gov). This algorithm involves first identifying high scoring sequence pair (HSPs) by identifying short words of length Win the query sequence that either match or satisfy some positive-valued threshold score T when aligned with a word of the same length in a database sequence. T is referred to as the neighborhood word score threshold (Altschul et al., supra). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide sequences would occur by chance. For example, a nucleic acid is considered similar to another if the smallest sum probability in comparison of the test nucleic acid to the other nucleic acid is less than about 1, less than about 0.1, less than about 0.01, and less than about 0.001. Two single-stranded polynucleotides are “the complement” of each other if their sequences can be aligned in an anti-parallel orientation such that every nucleotide in one polynucleotide is opposite its complementary nucleotide in the other polynucleotide, without the introduction of gaps, and without unpaired nucleotides at the 5′ or the 3′ end of either sequence. A polynucleotide is “complementary” to another polynucleotide if the two polynucleotides can hybridize to one another under moderately stringent conditions. Thus, a polynucleotide can be complementary to another polynucleotide without being its complement.

The term “functional fragment” means any portion of a polypeptide or nucleic acid sequence from which the respective full-length polypeptide or nucleic acid relates that is of a sufficient length and has a sufficient structure to confer a biological affect that is at least similar or substantially similar to the full-length polypeptide or nucleic acid upon which the fragment is based. In some embodiments, a functional fragment is a portion of a full-length or wild-type nucleic acid sequence that encodes any one of the nucleic acid sequences disclosed herein, and said portion encodes a polypeptide of a certain length and/or structure that is less than full-length but encodes a domain that still biologically functional as compared to the full-length or wild-type protein. In some embodiments, the functional fragment may have a reduced biological activity, about equivalent biological activity, or an enhanced biological activity as compared to the wild-type or full-length polypeptide sequence upon which the fragment is based. In some embodiments, the functional fragment is derived from the sequence of an organism, such as a human. In such embodiments, the functional fragment may retain 99%, 98%, 97%, 96%, 95%, 94%, 93%, 92%, 91%, or 90% sequence identity to the wild-type human sequence upon which the sequence is derived. In some embodiments, the functional fragment may retain 85%, 80%, 75%, 70%, 65%, or 60% sequence identity to the wild-type sequence upon which the sequence is derived.

By “fragment” is meant a portion of a polypeptide or nucleic acid molecule. This portion contains, preferably, at least about 5%, 10%, 20%, 30%, 40%, 50%, 60%, 70%, 80%, or about 90% of the entire length of the reference nucleic acid molecule or polypeptide. A fragment may contain about 5, 10, 20, 30, 40, 50, 60, 70, 80, 90, or 100, 200, 300, 400, 500, 600, 700, 800, 900, 1000 or more nucleotides or amino acids.

“Variants” is intended to mean substantially similar sequences. For nucleic acid molecules, a variant comprises a nucleic acid molecule having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” nucleic acid molecule or polypeptide comprises a naturally occurring nucleotide sequence or amino acid sequence, respectively. For nucleic acid molecules, conservative variants include those sequences that, because of the degeneracy of the genetic code, encode the amino acid sequence of one of the polypeptides of the disclosure. Variant nucleic acid molecules also include synthetically derived nucleic acid molecules, such as those generated, for example, by using site-directed mutagenesis but which still encode a protein of the disclosure. Generally, variants of a particular nucleic acid molecule of the disclosure will have at least about 70%, 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein. Variants of a particular nucleic acid molecule of the disclosure (i.e., the nucleic acids that encodes any amino acids in Tables 1, 2 or 3) can also be evaluated by comparison of the percent sequence identity between the polypeptide encoded by a variant nucleic acid molecule and the polypeptide encoded by the reference nucleic acid molecule. Percent sequence identity between any two polypeptides can be calculated using sequence alignment programs and parameters described elsewhere herein. Where any given pair of nucleic acid molecule of the disclosure is evaluated by comparison of the percent sequence identity shared by the two polypeptides that they encode, the percent sequence identity between the two encoded polypeptides is at least about 70%, 75%, 80%, 85%, 90%, 91%, 92%, 93%, 94%, 95%, 96%, 97%, 98%, 99% or more sequence identity. In some embodiments, the term “variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Variant proteins encompassed by the present disclosure are biologically active, that is they continue to possess the desired biological activity of the native protein as described herein. Such variants may result from, for example, genetic polymorphism or from human manipulation. Biologically active variants of a protein of the disclosure will have at least about 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the disclosure may differ from that protein by as few as 1-15 amino acid residues, as few as 1-10, such as 6-10, as few as 5, as few as 4, 3, 2, or even 1 amino acid residue. The proteins or polypeptides of the disclosure may be altered in various ways including amino acid substitutions, deletions, truncations, and insertions. Methods for such manipulations are generally known in the art. For example, amino acid sequence variants and fragments of the proteins can be prepared by mutations in the nucleic acid sequence that encode the amino acid sequence recombinantly. In some embodiments, the disclosure relates to cells comprising variants of those amino acids (or nucleic acid sequences encoding amino acid sequences) identified in Tables 1, 2 or 3.

“Optional” or “optionally” means that the subsequently described event, circumstance, or material may or may not occur or be present, and that the description includes instances where the event, circumstance, or material occurs or is present and instances where it does not occur or is not present.

In some embodiments, the disclosure relates to a system comprising a culture vessel comprising a hydrogel and one or a plurality of isolated stem cells and/or neural crest cells. In some embodiments, the culture vessel comprises Schwann cells. In some embodiments, the culture vessel comprises Schwann cells differentiated in culture from about 12 to about 20 days. In some embodiments, the culture vessel comprises a hydrogel and one or a plurality of isolated pluripotent stem cells and tissue culture medium comprising FGF2 or a functional fragment or variant thereof.

### Compositions of Matter

The disclosure relates to Schwann cells (SC) and, in some embodiments, compositions comprising the same. In some embodiments, SC cells of the present disclosure express CD98. In some embodiments, the cells comprise a nucleic acid sequence encoding or an amino acid sequence comprising CD98, or an amino acid sequence comprising at least about 70%, 75%, 80%, 85%, 90%, 91%, 92%, 93%, 94%, 95%, 96%, 97%, 98%, or 99% sequence identity to SEQ ID NO: 1, or a functional fragment that comprises at least about 70%, 75%, 80%, 85%, 90%, 91%, 92%, 93%, 94%, 95%, 96%, 97%, 98%, or 99% sequence identity to SEQ ID NO: 1.

In some embodiments, SC cells of the present disclosure express S100. In some embodiments, the SC cells comprise a nucleic acid sequence encoding or an amino acid sequence comprising S100, or an amino acid sequence comprising at least about 70%, 75%, 80%, 85%, 90%, 91%, 92%, 93%, 94%, 95%, 96%, 97%, 98%, or 99% sequence identity to SEQ ID NO: 2 or a functional fragment that comprises at least about 70%, 75%, 80%, 85%, 90%, 91%, 92%, 93%, 94%, 95%, 96%, 97%, 98%, or 99% sequence identity to SEQ ID NO: 2.

In some embodiments, SC cells of the present disclosure express MBP. In some embodiments, the SC cells comprise a nucleic acid sequence encoding or an amino acid sequence comprising MBP or an amino acid sequence comprising at least about 70%, 75%, 80%, 85%, 90%, 91%, 92%, 93%, 94%, 95%, 96%, 97%, 98%, or 99% sequence identity to SEQ ID NO: 3 or a functional fragment that comprises at least about 70%, 75%, 80%, 85%, 90%, 91%, 92%, 93%, 94%, 95%, 96%, 97%, 98%, or 99% sequence identity to SEQ ID NO: 3.

In some embodiments, SC cells of the present disclosure express GFAP. In some embodiments, the SC cells comprise a nucleic acid sequence encoding or an amino acid sequence comprising GFAP, or an amino acid sequence comprising at least about 70%, 75%, 80%, 85%, 90%, 91%, 92%, 93%, 94%, 95%, 96%, 97%, 98%, or 99% sequence identity to SEQ ID NO: 4 or a functional fragment that comprises at least about 70%, 75%, 80%, 85%, 90%, 91%, 92%, 93%, 94%, 95%, 96%, 97%, 98%, or 99% sequence identity to SEQ ID NO: 4.

In some embodiments, SC cells of the present disclosure express PMP22. In some embodiments, the SC cells comprise a nucleic acid sequence encoding or an amino acid sequence comprising PMP22, or an amino acid sequence comprising at least about 70%, 75%, 80%, 85%, 90%, 91%, 92%, 93%, 94%, 95%, 96%, 97%, 98%, or 99% sequence identity to SEQ ID NO: 5 or a functional fragment that comprises at least about 70%, 75%, 80%, 85%, 90%, 91%, 92%, 93%, 94%, 95%, 96%, 97%, 98%, or 99% sequence identity to SEQ ID NO: 5.

In some embodiments, SC cells of the present disclosure express CD6. In some embodiments, the SC cells comprise a nucleic acid sequence encoding or an amino acid sequence comprising CD6, or an amino acid sequence comprising at least about 70%, 75%, 80%, 85%, 90%, 91%, 92%, 93%, 94%, 95%, 96%, 97%, 98%, or 99% sequence identity to SEQ ID NO: 6 or a functional fragment that comprises at least about 70%, 75%, 80%, 85%, 90%, 91%, 92%, 93%, 94%, 95%, 96%, 97%, 98%, or 99% sequence identity to SEQ ID NO: 6.

In some embodiments, SC cells of the present disclosure express CD9. In some embodiments, the SC cells comprise a nucleic acid sequence encoding or an amino acid sequence comprising CD9 or an amino acid sequence comprising at least about 70%, 75%, 80%, 85%, 90%, 91%, 92%, 93%, 94%, 95%, 96%, 97%, 98%, or 99% sequence identity to SEQ ID NO: 7 or a functional fragment that comprises at least about 70%, 75%, 80%, 85%, 90%, 91%, 92%, 93%, 94%, 95%, 96%, 97%, 98%, or 99% sequence identity to SEQ ID NO: 7.

In some embodiments, SC cells of the present disclosure express CD44. In some embodiments, the SC cells comprise a nucleic acid sequence encoding or an amino acid sequence comprising CD44 or an amino acid sequence comprising at least about 70%, 75%, 80%, 85%, 90%, 91%, 92%, 93%, 94%, 95%, 96%, 97%, 98%, or 99% sequence identity to SEQ ID NO: 8 or a functional fragment that comprises at least about 70%, 75%, 80%, 85%, 90%, 91%, 92%, 93%, 94%, 95%, 96%, 97%, 98%, or 99% sequence identity to SEQ ID NO: 8.

In some embodiments, SC cells of the present disclosure express CD46. In some embodiments, the SC cells comprise a nucleic acid sequence encoding or an amino acid sequence comprising CD46, or an amino acid sequence comprising at least about 70%, 75%, 80%, 85%, 90%, 91%, 92%, 93%, 94%, 95%, 96%, 97%, 98%, or 99% sequence identity to SEQ ID NO: 9 or a functional fragment that comprises at least about 70%, 75%, 80%, 85%, 90%, 91%, 92%, 93%, 94%, 95%, 96%, 97%, 98%, or 99% sequence identity to SEQ ID NO: 9.

In some embodiments, SC cells of the present disclosure express CD49e. In some embodiments, the SC cells comprise a nucleic acid sequence encoding or an amino acid sequence comprising CD49e, or an amino acid sequence comprising at least about 70%, 75%, 80%, 85%, 90%, 91%, 92%, 93%, 94%, 95%, 96%, 97%, 98%, or 99% sequence identity to SEQ ID NO: 10 or a functional fragment that comprises at least about 70%, 75%, 80%, 85%, 90%, 91%, 92%, 93%, 94%, 95%, 96%, 97%, 98%, or 99% sequence identity to SEQ ID NO: 10.

In some embodiments, SC cells of the present disclosure express CD81. In some embodiments, the SC cells comprise a nucleic acid sequence encoding or an amino acid sequence comprising CD81, or an amino acid sequence comprising at least about 70%, 75%, 80%, 85%, 90%, 91%, 92%, 93%, 94%, 95%, 96%, 97%, 98%, or 99% sequence identity to SEQ ID NO: 11 or a functional fragment that comprises at least about 70%, 75%, 80%, 85%, 90%, 91%, 92%, 93%, 94%, 95%, 96%, 97%, 98%, or 99% sequence identity to SEQ ID NO: 11.

In some embodiments, SC cells of the present disclosure express CD146. In some embodiments, the SC cells comprise a nucleic acid sequence encoding or an amino acid sequence comprising CD146, or an amino acid sequence comprising at least about 70%, 75%, 80%, 85%, 90%, 91%, 92%, 93%, 94%, 95%, 96%, 97%, 98%, or 99% sequence identity to SEQ ID NO: 12 or a functional fragment that comprises at least about 70%, 75%, 80%, 85%, 90%, 91%, 92%, 93%, 94%, 95%, 96%, 97%, 98%, or 99% sequence identity to SEQ ID NO: 12.

In some embodiments, SC cells of the present disclosure express CD147. In some embodiments, the SC cells comprise a nucleic acid sequence encoding or an amino acid sequence comprising CD147, or an amino acid sequence comprising at least about 70%, 75%, 80%, 85%, 90%, 91%, 92%, 93%, 94%, 95%, 96%, 97%, 98%, or 99% sequence identity to SEQ ID NO: 13 or a functional fragment that comprises at least about 70%, 75%, 80%, 85%, 90%, 91%, 92%, 93%, 94%, 95%, 96%, 97%, 98%, or 99% sequence identity to SEQ ID NO: 13.

In some embodiments, SC cells of the present disclosure express CD166. In some embodiments, the SC cells comprise a nucleic acid sequence encoding or an amino acid sequence comprising CD166, or an amino acid sequence comprising at least about 70%, 75%, 80%, 85%, 90%, 91%, 92%, 93%, 94%, 95%, 96%, 97%, 98%, or 99% sequence identity to SEQ ID NO: 14 or a functional fragment that comprises at least about 70%, 75%, 80%, 85%, 90%, 91%, 92%, 93%, 94%, 95%, 96%, 97%, 98%, or 99% sequence identity to SEQ ID NO: 14.

In some embodiments, SC cells of the present disclosure express CD171. In some embodiments, the SC cells comprise a nucleic acid sequence encoding or an amino acid sequence comprising CD171 or an amino acid sequence comprising at least about 70%, 75%, 80%, 85%, 90%, 91%, 92%, 93%, 94%, 95%, 96%, 97%, 98%, or 99% sequence identity to SEQ ID NO: 15 or a functional fragment that comprises at least about 70%, 75%, 80%, 85%, 90%, 91%, 92%, 93%, 94%, 95%, 96%, 97%, 98%, or 99% sequence identity to SEQ ID NO: 15.

In some embodiments, SC cells of the present disclosure express NGFR. In some embodiments, the SC cells comprise a nucleic acid sequence encoding or an amino acid sequence comprising NGFR or an amino acid sequence comprising at least about 70%, 75%, 80%, 85%, 90%, 91%, 92%, 93%, 94%, 95%, 96%, 97%, 98%, or 99% sequence identity to SEQ ID NO: 16 or a functional fragment that comprises at least about 70%, 75%, 80%, 85%, 90%, 91%, 92%, 93%, 94%, 95%, 96%, 97%, 98%, or 99% sequence identity to SEQ ID NO: 16.

In some embodiments, SC cells of the present disclosure express SOX10. In some embodiments, the SC cells comprise a nucleic acid sequence encoding or an amino acid sequence comprising SOX10, or an amino acid sequence comprising at least about 70%, 75%, 80%, 85%, 90%, 91%, 92%, 93%, 94%, 95%, 96%, 97%, 98%, or 99% sequence identity to SEQ ID NO: 17 or a functional fragment that comprises at least about 70%, 75%, 80%, 85%, 90%, 91%, 92%, 93%, 94%, 95%, 96%, 97%, 98%, or 99% sequence identity to SEQ ID NO: 17.

In some embodiments, SC cells of the present disclosure express POU3F2. In some embodiments, the SC cells comprise a nucleic acid sequence encoding or an amino acid sequence comprising POU3F2 or an amino acid sequence comprising at least about 70%, 75%, 80%, 85%, 90%, 91%, 92%, 93%, 94%, 95%, 96%, 97%, 98%, or 99% sequence identity to SEQ ID NO: 18 or a functional fragment that comprises at least about 70%, 75%, 80%, 85%, 90%, 91%, 92%, 93%, 94%, 95%, 96%, 97%, 98%, or 99% sequence identity to SEQ ID NO: 18.

In some embodiments, SC cells of the present disclosure express MPZ. In some embodiments, the SC cells comprise a nucleic acid sequence encoding or an amino acid sequence comprising MPZ or an amino acid sequence comprising at least about 70%, 75%, 80%, 85%, 90%, 91%, 92%, 93%, 94%, 95%, 96%, 97%, 98%, or 99% sequence identity to SEQ ID NO: 19 or a functional fragment that comprises at least about 70%, 75%, 80%, 85%, 90%, 91%, 92%, 93%, 94%, 95%, 96%, 97%, 98%, or 99% sequence identity to SEQ ID NO: 19.

In some embodiments, SC cells of the present disclosure express GAP43. In some embodiments, the SC cells comprise a nucleic acid sequence encoding or an amino acid sequence comprising GAP43, or an amino acid sequence comprising at least about 70%, 75%, 80%, 85%, 90%, 91%, 92%, 93%, 94%, 95%, 96%, 97%, 98%, or 99% sequence identity to SEQ ID NO: 20 or a functional fragment that comprises at least about 70%, 75%, 80%, 85%, 90%, 91%, 92%, 93%, 94%, 95%, 96%, 97%, 98%, or 99% sequence identity to SEQ ID NO: 20.

In some embodiments, SC cells of the present disclosure express ERBB3. In some embodiments, the SC cells comprise a nucleic acid sequence encoding or an amino acid sequence comprising ERBB3, or an amino acid sequence comprising at least about 70%, 75%, 80%, 85%, 90%, 91%, 92%, 93%, 94%, 95%, 96%, 97%, 98%, or 99% sequence identity to SEQ ID NO: 21 or a functional fragment that comprises at least about 70%, 75%, 80%, 85%, 90%, 91%, 92%, 93%, 94%, 95%, 96%, 97%, 98%, or 99% sequence identity to SEQ ID NO: 21.

In some embodiments, SC cells of the present disclosure express GDNF. In some embodiments, the SC cells comprise a nucleic acid sequence encoding or an amino acid sequence comprising GDNF, or an amino acid sequence comprising at least about 70%, 75%, 80%, 85%, 90%, 91%, 92%, 93%, 94%, 95%, 96%, 97%, 98%, or 99% sequence identity to SEQ ID NO: 22 or a functional fragment that comprises at least about 70%, 75%, 80%, 85%, 90%, 91%, 92%, 93%, 94%, 95%, 96%, 97%, 98%, or 99% sequence identity to SEQ ID NO: 22.

In some embodiments, SC cells of the present disclosure express MAG. In some embodiments, the SC cells comprise a nucleic acid sequence encoding or an amino acid sequence comprising MAG, or an amino acid sequence comprising at least about 70%, 75%, 80%, 85%, 90%, 91%, 92%, 93%, 94%, 95%, 96%, 97%, 98%, or 99% sequence identity to SEQ ID NO: 23 or a functional fragment that comprises at least about 70%, 75%, 80%, 85%, 90%, 91%, 92%, 93%, 94%, 95%, 96%, 97%, 98%, or 99% sequence identity to SEQ ID NO: 23.

In some embodiments, SC cells of the present disclosure express PLLP. In some embodiments, the SC cells comprise a nucleic acid sequence encoding or an amino acid sequence comprising PLLP, or an amino acid sequence comprising at least about 70%, 75%, 80%, 85%, 90%, 91%, 92%, 93%, 94%, 95%, 96%, 97%, 98%, or 99% sequence identity to SEQ ID NO: 24 or a functional fragment that comprises at least about 70%, 75%, 80%, 85%, 90%, 91%, 92%, 93%, 94%, 95%, 96%, 97%, 98%, or 99% sequence identity to SEQ ID NO: 24.

In some embodiments, SC cells of the present disclosure express POU6F2. In some embodiments, the SC cells comprise a nucleic acid sequence encoding or an amino acid sequence comprising POU6F2, or an amino acid sequence comprising at least about 70%, 75%, 80%, 85%, 90%, 91%, 92%, 93%, 94%, 95%, 96%, 97%, 98%, or 99% sequence identity to SEQ ID NO: 25 or a functional fragment that comprises at least about 70%, 75%, 80%, 85%, 90%, 91%, 92%, 93%, 94%, 95%, 96%, 97%, 98%, or 99% sequence identity to SEQ ID NO: 25.

In some embodiments, SC cells of the present disclosure express PLXNB3. In some embodiments, the SC cells comprise a nucleic acid sequence encoding or an amino acid sequence comprising PLXNB3, or an amino acid sequence comprising at least about 70%, 75%, 80%, 85%, 90%, 91%, 92%, 93%, 94%, 95%, 96%, 97%, 98%, or 99% sequence identity to SEQ ID NO: 26 or a functional fragment that comprises at least about 70%, 75%, 80%, 85%, 90%, 91%, 92%, 93%, 94%, 95%, 96%, 97%, 98%, or 99% sequence identity to SEQ ID NO: 26.

In some embodiments, SC cells of the present disclosure express ERBB3, or an amino acid sequence comprising at least about 70%, 75%, 80%, 85%, 90%, 91%, 92%, 93%, 94%, 95%, 96%, 97%, 98%, or 99% sequence identity to SEQ ID NO: 27 or a functional fragment that comprises at least about 70%, 75%, 80%, 85%, 90%, 91%, 92%, 93%, 94%, 95%, 96%, 97%, 98%, or 99% sequence identity to SEQ ID NO: 27.

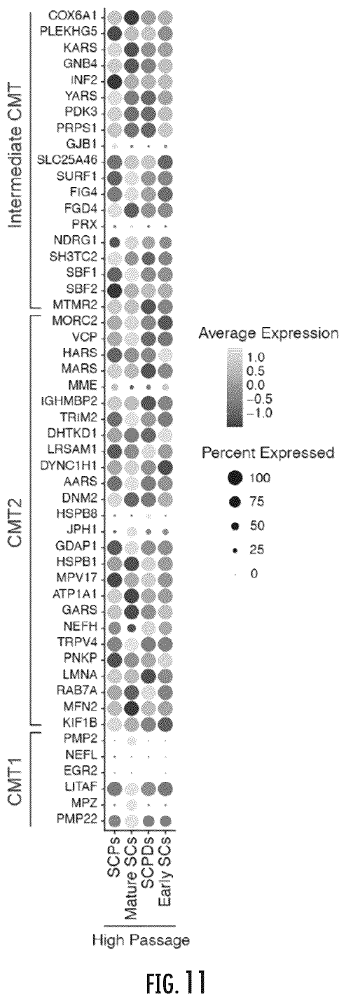

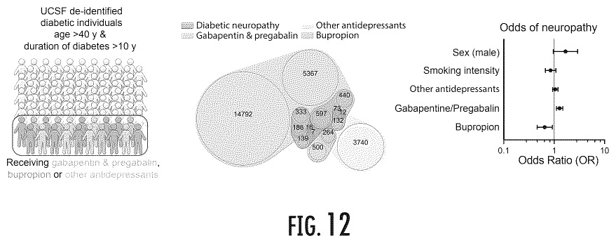

In some embodiments, the SC cells comprise a nucleic acid sequence encoding or an amino acid sequence comprising one or a combination of proteins identified in FIG. 11, or an amino acid sequence comprising at least about 70%, 75%, 80%, 85%, 90%, 91%, 92%, 93%, 94%, 95%, 96%, 97%, 98%, or 99% sequence identity to one or a combination of nucleic acids encoding one or a combination of amino acids identified in FIG. 11. or a functional fragment that comprises at least about 70%, 75%, 80%, 85%, 90%, 91%, 92%, 93%, 94%, 95%, 96%, 97%, 98%, or 99% sequence identity to one or a combination of nucleic acids encoding one or a combination of amino acids identified in FIG. 11.

Glial fibrillary acidic protein (GFAP) is a class-III intermediate filament. During the development of the central nervous system, GFAP is a cell-specific marker that distinguishes astrocytes from other glial cells. A non-limiting example of GFAP is the GFAP from human (Homo sapiens, UniProt accession No. P14136) having the following sequence:

(SEQ ID NO: 53)
MERRRITSAARRSYVSSGEMMVGGLAPGRRLGPGTRLSLARMPPPLPTRV
DFSLAGALNAGFKETRASERAEMMELNDRFASYIEKVRFLEQQNKALAAE
LNQLRAKEPTKLADVYQAELRELRLRLDQLTANSARLEVERDNLAQDLAT
VRQKLQDETNLRLEAENNLAAYRQEADEATLARLDLERKIESLEEEIRFL
RKIHEEEVRELQEQLARQQVHVELDVAKPDLTAALKEIRTQYEAMASSNM
HEAEEWYRSKFADLTDAAARNAELLRQAKHEANDYRRQLQSLTCDLESLR
GTNESLERQMREQEERHVREAASYQEALARLEEEGQSLKDEMARHLQEYQ
DLLNVKLALDIEIATYRKLLEGEENRITIPVQTFSNLQIRETSLDTKSVS
EGHLKRNIVVKTVEMRDGEVIKESKQEHKDVM.

Another non-limiting example of GFAP is the GFAP from rat (Rattus norvegicus; UniProt accession No. P47819) having the following sequence:

(SEQ ID NO: 54)
MERRRITSARRSYASSETMVRGHGPTRHLGTIPRLSLSRMTPPLPARVDF
SLAGALNAGFKETRASERAEMMELNDRFASYIEKVRFLEQQNKALAAELN
QLRAKEPTKLADVYQAELRELRLRLDQLTTNSARLEVERDNLTQDLGTLR
QKLQDETNLRLEAENNLAVYRQEADEATLARVDLERKVESLEEEIQFLRK
IHEEEVRELQEQLAQQQVHVEMDVAKPDLTAALREIRTQYEAVATSNMQE
TEEWYRSKFADLTDVASRNAELLRQAKHEANDYRRQLQALTCDLESLRGT
NESLERQMREQEERHARESASYQEALARLEEEGQSLKEEMARHLQEYQDL
LNVKLALDIEIATYRKLLEGEENRITIPVQTFSNLQIRETSLDTKSVSEG
HLKRNIVVKTVEMRDGEVIKESKQEHKDVM.

A further non-limiting example of GFAP is the GFAP from mouse (Mus musculus; UniProt accession No. P03995) having the following sequence:

(SEQ ID NO: 55)
MERRRITSARRSYASETVVRGLGPSRQLGTMPRFSLSRMTPPLPARVDFS
LAGALNAGFKETRASERAEMMELNDRFASYIEKVRFLEQQNKALAAELNQ
LRAKEPTKLADVYQAELRELRLRLDQLTANSARLEVERDNFAQDLGTLRQ
KLQDETNLRLEAENNLAAYRQEADEATLARVDLERKVESLEEEIQFLRKI
YEEEVRELREQLAQQQVHVEMDVAKPDLTAALREIRTQYEAVATSNMQET
EEWYRSKFADLTDAASRNAELLRQAKHEANDYRRQLQALTCDLESLRGTN
ESLERQMREQEERHARESASYQEALARLEEEGQSLKEEMARHLQEYQDLL
NVKLALDIEIATYRKLLEGEENRITIPVQTFSNLQIRETSLDTKSVSEGH
LKRNIVVKTVEMRDGEVIKDSKQEHKDVVM.

In some embodiments therefore, GFAP comprises at least about 70% sequence identity to SEQ ID NO: 53, SEQ ID NO: 54 or SEQ ID NO: 55, or a functional fragment thereof. In some embodiments, GFAP comprises at least about 75%, 80%, 85%, 90%, 95%, 96%, 97%, 98% or 99% sequence identity to SEQ ID NO: 53, SEQ ID NO: 54 or SEQ ID NO: 55, or a functional fragment thereof. In some embodiments, GFAP comprises SEQ ID NO: 53, SEQ ID NO: 54 or SEQ ID NO: 55, or a functional fragment thereof.

Enteric neural crest cells express transcription factor SOX10, which directs the activity of other genes that signal neural crest cells to become more specific cell types including enteric nerves. A non-limiting example of SOX10 is the SOX10 from human (Homo sapiens, UniProt accession No. P56693) having the following sequence:

(SEQ ID NO: 56)
MAEEQDLSEVELSPVGSEEPRCLSPGSAPSLGPDGGGGGSGLRASPGPGE
LGKVKKEQQDGEADDDKFPVCIREAVSQVLSGYDWTLVPMPVRVNGASKS
KPHVKRPMNAFMVWAQAARRKLADQYPHLHNAELSKTLGKLWRLLNESDK
RPFIEEAERLRMQHKKDHPDYKYQPRRRKNGKAAQGEAECPGGEAEQGGT
AAIQAHYKSAHLDHRHPGEGSPMSDGNPEHPSGQSHGPPTPPTTPKTELQ
SGKADPKRDGRSMGEGGKPHIDFGNVDIGEISHEVMSNMETFDVAELDQY
LPPNGHPGHVSSYSAAGYGLGSALAVASGHSAWISKPPGVALPTVSPPGV
DAKAQVKTETAGPQGPPHYTDQPSTSQIAYTSLSLPHYGSAFPSISRPQF
DYSDHQPSGPYYGHSGQASGLYSAFSYMGPSQRPLYTAISDPSPSGPQSH
SPTHWEQPVYTTLSRP.

Another non-limiting example of SOX10 is the SOX10 from rat (Rattus norvegicus; UniProt accession No. 055170) having the following sequence:

(SEQ ID NO: 57)
MAEEQDLSEVELSPVGSEEPRCLSPSSAPSLGPDGGGGGSGLRASPGPGE
LGKVKKEQQDGEADDDKFPVCIREAVSQVLSGYDWTLVPMPVRVNGASKS
KPHVKRPMNAFMVWAQAARRKLADQYPHLHNAELSKTLGKLWRLLNESDK
RPFIEEAERLRMQHKKDHPDYKYQPRRRKNGKAAQGEAECPGGETDQGGA
AAIQAHYKSAHLDHRHPEEGSPMSDGNPEHPSGQSHGPPTPPTTPKTELQ
SGKADPKRDGRSLGEGGKPHIDFGNVDIGEISHEVMSNMETFDVTELDQY
LPPNGHPGHVGSYSAAGYGLSSALAVASGHSAWISKPPGVALPTVSPPAV
DAKAQVKTETTGPQGPPHYTDQPSTSQIAYTSLSLPHYGSAFPSISRPQF
DYSDHQPSGPYYGHAGQASGLYSAFSYMGPSQRPLYTAISDPSPSGPQSH
SPTHWEQPVYTTLSRP.

A further non-limiting example of SOX10 is the SOX10 from mouse (Mus musculus; UniProt accession No. Q04888) having the following sequence:

(SEQ ID NO: 58)
MAEEQDLSEVELSPVGSEEPRCLSPGSAPSLGPDGGGGGSGLRASPGPGE
LGKVKKEQQDGEADDDKFPVCIREAVSQVLSGYDWTLVPMPVRVNGASKS
KPHVKRPMNAFMVWAQAARRKLADQYPHLHNAELSKTLGKLWRLLNESDK
RPFIEEAERLRMQHKKDHPDYKYQPRRRKNGKAAQGEAECPGGEAEQGGA
AAIQAHYKSAHLDHRHPEEGSPMSDGNPEHPSGQSHGPPTPPTTPKTELQ
SGKADPKRDGRSLGEGGKPHIDFGNVDIGEISHEVMSNMETFDVTELDQY
LPPNGHPGHVGSYSAAGYGLGSALAVASGHSAWISKPPGVALPTVSPPGV
DAKAQVKTETTGPQGPPHYTDQPSTSQIAYTSLSLPHYGSAFPSISRPQF
DYSDHQPSGPYYGHAGQASGLYSAFSYMGPSQRPLYTAISDPSPSGPQSH
SPTHWEQPVYTTLSRP.

In some embodiments therefore, SOX10 comprises at least about 70% sequence identity to SEQ ID NO: 56, SEQ ID NO: 57 or SEQ ID NO: 58, or a functional fragment thereof. In some embodiments, SOX10 comprises at least about 75%, 80%, 85%, 90%, 95%, 96%, 97%, 98% or 99% sequence identity to SEQ ID NO: 16, SEQ ID NO: 17 or SEQ ID NO: 18, or a functional fragment thereof. In some embodiments, SOX10 comprises SEQ ID NO: 56, SEQ ID NO: 57 or SEQ ID NO: 58, or a functional fragment thereof.

TABLE 1

| SEQ ID NO: | AMINO ACID SEQUENCE |
|---|---|
| CD98 | MAGAGPKRRA LAAPAAEEKE EAREKMLAAK SADGSAPAGE GEGVTLQRNI |
| SEQ ID NO: 1 | TLLNGVAIIV GTIIGSGIFV TPTGVLKEAG SPGLALVVWA ACGVFSIVGA |
| | LCYAELGTTI SKSGGDYAYM LEVYGSLPAF LKLWIELLII RPSSQYIVAL |
| | VFATYLLKPL FPTCPVPEEA AKLVACLCVL LLTAVNCYSV KAATRVQDAF |
| | AAAKLLALAL IILLGFVQIG KGDVSNLDPN FSFEGTKLDV GNIVLALYSG |
| | LFAYGGWNYL NFVTEEMINP YRNLPLAIII SLPIVTLVYV LTNLAYFTTL |
| | STEQMLSSEA VAVDFGNYHL GVMSWIIPVF VGLSCFGSVN GSLFTSSRLF |
| | FVGSREGHLP SILSMIHPQL LTPVPSLVFT CVMTLLYAFS KDIFSVINFF |
| | SFFNWLCVAL AIIGMIWLRH RKPELERPIK VNLALPVFFI LACLFLIAVS |
| | FWKTPVECGI GFTIILSGLP VYFFGVWWKN KPKWLLQGIF STTVLCQKLM QVVPQET |
| S100 | MGSELETAME TLINVFHAHS GKEGDKYKLS KKELKELLQT ELSGFLDAQK |
| SEQ ID NO: 2 | DVDAVDKVMK ELDENGDGEV DFQEYVVLVA ALTVACNNFF WENS |
| MBP | MASQKRPSQR HGSKYLATAS TMDHARHGFL PRHRDTGILD SIGRFFGGDR |
| SEQ ID NO: 3 | GAPKRGSGKV PWLKPGRSPL PSHARSQPGL CNMYKDSHHP ARTAHYGSLP |
| | QKSHGRTQDE NPVVHFFKNI VTPRTPPPSQ GKGRGLSLSR FSWGAEGQRP |
| | GFGYGGRASD YKSAHKGFKG VDAQGTLSKI FKLGGRDSRS GSPMARR |
| GFAP | MERRRITSAA RRSYVSSGEM MVGGLAPGRR LGPGTRLSLA RMPPPLPTRV |
| SEQ ID NO: 4 | DFSLAGALNA GFKETRASER AEMMELNDRF ASYIEKVRFL EQQNKALAAE |
| | LNQLRAKEPT KLADVYQAEL RELRLRLDQL TANSARLEVE RDNLAQDLAT |
| | VRQKLQDETN LRLEAENNLA AYRQEADEAT LARLDLERKI ESLEEEIRFL |
| | RKIHEEEVRE LQEQLARQQV HVELDVAKPD LTAALKEIRT QYEAMASSNM |
| | HEAEEWYRSK FADLTDAAAR NAELLRQAKH EANDYRRQLQ SLTCDLESLR |
| | GTNESLERQM REQEERHVRE AASYQEALAR LEEEGQSLKD EMARHLQEYQ |
| | DLLNVKLALD IEIATYRKLL EGEENRITIP VQTFSNLQIR GGKSTKDGEN |
| | HKVTRYLKSL TIRVIPIQAH QIVNGTPPAR ETSLDTKSVS EGHLKRNIVV |
| | KTVEMRDGEV IKESKQEHKD VM |
| PMP22 | MLLLLLSIIV LHVAVLVLLF VSTIVSQWIV GNGHATDLWQ NCSTSSSGNV |
| SEQ ID NO: 5 | HHCFSSSPNE WLQSVQATMI LSIIFSILSL FLFFCQLFTL TKGGRFYITG IFQILAGLCV |
| | MSAAAIYTVR HPEWHLNSDY SYGFAYILAW VAFPLALLSG VIYVILRKRE |
| CD6 | MWLFFGITGL LTAALSGHPS PAPPDQLNTS SAESELWEPG ERLPVRLTNG |
| SEQ ID NO: 6 | SSSCSGTVEV RLEASWEPAC GALWDSRAAE AVCRALGCGG AEAASQLAPP |
| | TPELPPPPAA GNTSVAANAT LAGAPALLCS GAEWRLCEVV EHACRSDGRR |
| | ARVTCAENRA LRLVDGGGAC AGRVEMLEHG EWGSVCDDTW DLEDAHVVCR |
| | QLGCGWAVQA LPGLHFTPGR GPIHRDQVNC SGAEAYLWDC PGLPGQHYCG |
| | HKEDAGAVCS EHQSWRLTGG ADRCEGQVEV HFRGVWNTVC DSEWYPSEAK |
| | VLCQSLGCGT AVERPKGLPH SLSGRMYYSC NGEELTLSNC SWRENNSNLC |
| | SQSLAARVLC SASRSLHNLS TPEVPASVQT VTIESSVTVK IENKESRELM LLIPSIVLGI |
| | LLLGSLIFIA FILLRIKGKY ALPVMVNHQH LPTTIPAGSN SYQPVPITIP KEVFMLPIQV |
| | QAPPPEDSDS GSDSDYEHYD FSAQPPVALT TFYNSQRHRV TDEEVQQSRF |
| | QMPPLEEGLE ELHASHIPTA NPGHCITDPP SLGPQYHPRS NSESSTSSGE |
| | DYCNSPKSKL PPWNPQVFSS ERSSFLEQPP NLELAGTQPA FSAGPPADDS |
| | SSTSSGEWYQ NFQPPPQPPS EEQFGCPGSP SPQPDSTDND DYDDISAA |
| CD9 | MPVKGGTKCI KYLLFGFNFI FWLAGIAVLA IGLWLRFDSQ TKSIFEQETN |
| SEQ ID NO: 7 | NNNSSFYTGV YILIGAGALM MLVGFLGCCG AVQESQCMLG LFFGFLLVIF |
| | AIEIAAAIWG YSHKDEVIKE VQEFYKDTYN KLKTKDEPQR ETLKAIHYAV |
| | CRLGKDTLLR FLRIVSAHRV LTAPDQCKHS NRLLTICFSH PHPCLLAVVE |
| | LLWFGWGRGT VYLRHLPQEG RTRNLHREGK LRPGSWCPCP HCSGQTLQA |
| CD44 | MDKFWWHAAW GLCLVPLSLA QIDLNITCRF AGVFHVEKNG RYSISRTEAA |
| SEQ ID NO: 8 | DLCKAFNSTL PTMAQMEKAL SIGFETCRYG FIEGHVVIPR IHPNSICAAN |
| | NTGVYILTSN TSQYDTYCFN ASAPPEEDCT SVTDLPNAFD GPITITIVNR |
| | DGTRYVQKGE YRTNPEDIYP SNPTDDDVSS GSSSERSSTS GGYIFYTEST |
| | VHPIPDEDSP WITDSTDRIP ATTLMSTSAT ATETATKRQE TWDWFSWLFL |
| | PSESKNHLHT TTQMAGTSSN TISAGWEPNE ENEDERDRHL SFSGSGIDDD |
| | EDFISSTIST TPRAFDHTKQ NQDWTQWNPS HSNPEVLLQT TTRMTDVDRN |
| | GTTAYEGNWN PEAHPPLIHH EHHEEEETPH STSTIQATPS STTEETATQK |
| | EQWFGNRWHE GYRQTPKEDS HSTTGTAAAS AHTSHPMQGR TTPSPEDSSW |
| | TDFFNPISHP MGRGHQAGRR MDMDSSHSIT LQPTANPNTG LVEDLDRTGP |
| | LSMTTQQSNS QSFSTSHEGL EEDKDHPTTS TLTSSNRNDV TGGRRDPNHS |
| | EGSTTLLEGY TSHYPHTKES RTFIPVTSAK TGSFGVTAVT VGDSNSNVNR |
| | SLSGDQDTFH PSGGSHTTHG SESDGHSHGS QEGGANTTSG PIRTPQIPEW |
| | LIILASLLAL ALILAVCIAV NSRRRCGQKK KLVINSGNGA VEDRKPSGLN |
| | GEASKSQEMV HLVNKESSET PDQFMTADET RNLQNVDMKI GV |
| CD46 | MEPPGRRECP FPSWRFPGLL LAAMVLLLYS FSDACEEPPT FEAMELIGKP |
| SEQ ID NO: 9 | KPYYEIGERV DYKCKKGYFY IPPLATHTIC DRNHTWLPVS DDACYRETCP |
| | YIRDPLNGQA VPANGTYEFG YQMHFICNEG YYLIGEEILY CELKGSVAIW |
| | SGKPPICEKV LCTPPPKIKN GKHTFSEVEV FEYLDAVTYS CDPAPGPDPF |
| | SLIGESTIYC GDNSVWSRAA PECKVVKCRF PVVENGKQIS GFGKKFYYKA |
| | TVMFECDKGF YLDGSDTIVC DSNSTWDPPV PKCLKVLPPS STKPPALSHS |
| | VSTSSTTKSP ASSASGPRPT YKPPVSNYPG YPKPEEGILD SLDVWVIAVI |
| | VIAIVVGVAV ICVVPYRYLQ RRKKKGKADG GAEYATYQTK STTPAEQRG |
| CD49e | MGSRTPESPL HAVQLRWGPR RRPPLLPLLL LLLPPPPRVG GENLDAEAPA |
| SEQ ID NO: 10 | VLSGPPGSFF GFSVEFYRPG TDGVSVLVGA PKANTSQPGV LQGGAVYLCP |
| | WGASPTQCTP IEFDSKGSRL LESSLSSSEG EEPVEYKSLQ WFGATVRAHG |
| | SSILACAPLY SWRTEKEPLS DPVGTCYLST DNFTRILEYA PCRSDFSWAA |
| | GQGYCQGGFS AEFTKTGRVV LGGPGSYFWQ GQILSATQEQ IAESYYPEYL |
| | INLVQGQLQT RQASSIYDDS YLGYSVAVGE FSGDDTEDFV AGVPKGNLTY |
| | GYVTILNGSD IRSLYNFSGE QMASYFGYAV AATDVNGDGL DDLLVGAPLL |
| | MDRTPDGRPQ EVGRVYVYLQ HPAGIEPTPT LTLTGHDEFG RFGSSLTPLG |
| | DLDQDGYNDV AIGAPFGGET QQGVVFVFPG GPGGLGSKPS QVLQPLWAAS |
| | HTPDFFGSAL RGGRDLDGNG YPDLIVGSFG VDKAVVYRGR PIVSASASLT |
| | IFPAMENPEE RSCSLEGNPV ACINLSFCLN ASGKHVADSI GFTVELQLDW |
| | QKQKGGVRRA LFLASRQATL TQTLLIQNGA REDCREMKIY LRNESEFRDK |
| | LSPIHIALNF SLDPQAPVDS HGLRPALHYQ SKSRIEDKAQ ILLDCGEDNI |
| | CVPDLQLEVF GEQNHVYLGD KNALNLTFHA QNVGEGGAYE AELRVTAPPE |
| | AEYSGLVRHP GNFSSLSCDY FAVNQSRLLV CDLGNPMKAG ASLWGGLRFT |
| | VPHLRDTKKT IQFDFQILSK NLNNSQSDVV SFRLSVEAQA QVTLNGVSKP |
| | EAVLFPVSDW HPRDQPQKEE DLGPAVHHVY ELINQGPSSI SQGVLELSCP |
| | QALEGQQLLY VTRVTGLNCT TNHPINPKGL ELDPEGSLHH QQKREAPSRS |
| | SASSGPQILK CPEAECFRLR CELGPLHQQE SQSLQLHFRV WAKTFLQREH |
| | QPFSLQCEAV YKALKMPYRI LPRQLPQKER QVATAVQWTK AEGSYGVPLW |
| | IIILAILFGL LLLGLLIYIL YKLGFFKRSL PYGTAMEKAQ LKPPATSDA |
| CD81 | MGVEGCTKCI KYLLFVFNFV FWLAGGVILG VALWLRHDPQ TTNLLYLELG |
| SEQ ID NO: 11 | DKPAPNTFYV GIYILIAVGA VMMFVGFLGC YGAIQESQCL LGTFFTCLVI |
| | LFACEVAAGI WGFVNKDQIA KDVKQFYDQA LQQAVVDDDA NNAKAVVKTF |
| | HETLDCCGSS TLTALTTSVL KNNLCPSGSN IISNLFKEDC HQKIDDLFSG KLYLIGIAAI |
| | VVAVIMIFEM ILSMVLCCGI RNSSVY |
| CD146 | MGLPRLVCAF LLAACCCCPR VAGVPGEAEQ PAPELVEVEV GSTALLKCGL |
| SEQ ID NO: 12 | SQSQGNLSHV DWFSVHKEKR TLIFRVRQGQ GQSEPGEYEQ RLSLQDRGAT |
| | LALTQVTPQD ERIFLCQGKR PRSQEYRIQL RVYKAPEEPN IQVNPLGIPV |
| | NSKEPEEVAT CVGRNGYPIP QVIWYKNGRP LKEEKNRVHI QSSQTVESSG |
| | LYTLQSILKA QLVKEDKDAQ FYCELNYRLP SGNHMKESRE VTVPVFYPTE |
| | KVWLEVEPVG MLKEGDRVEI RCLADGNPPP HFSISKQNPS TREAEEETTN |
| | DNGVLVLEPA RKEHSGRYEC QGLDLDTMIS LLSEPQELLV NYVSDVRVSP |
| | AAPERQEGSS LTLTCEAESS QDLEFQWLRE ETGQVLERGP VLQLHDLKRE |
| | AGGGYRCVAS VPSIPGLNRT QLVNVAIFGP PWMAFKERKV WVKENMVLNL |
| | SCEASGHPRP TISWNVNGTA SEQDQDPQRV LSTLNVLVTP ELLETGVECT |
| | ASNDLGKNTS ILFLELVNLT TLTPDSNTTT GLSTSTASPH TRANSTSTER |
| | KLPEPESRGV VIVAVIVCIL VLAVLGAVLY FLYKKGKLPC RRSGKQEITL |
| | PPSRKSELVV EVKSDKLPEE MGLLQGSSGD KRAPGDQGEK YIDLRH |
| CD147 | MAAALFVLLG FALLGTHGAS GAAGFVQAPL SQQRWVGGSV ELHCEAVGSP |
| SEQ ID NO: 13 | VPEIQWWFEG QGPNDTCSQL WDGARLDRVH IHATYHQHAA STISIDTLVE |
| | EDTGTYECRA SNDPDRNHLT RAPRVKWVRA QAVVLVLEPG TVFTTVEDLG |
| | SKILLTCSLN DSATEVTGHR WLKGGVVLKE DALPGQKTEF KVDSDDQWGE |
| | YSCVFLPEPM GTANIQLHGP PRVKAVKSSE HINEGETAML VCKSESVPPV |
| | TDWAWYKITD SEDKALMNGS ESRFFVSSSQ GRSELHIENL NMEADPGQYR |
| | CNGTSSKGSD QAIITLRVRS HLAALWPFLG IVAEVLVLVT IIFIYEKRRK |
| | PEDVLDDDDA GSAPLKSSGQ HQNDKGKNVR QRNSS |
| CD166 | MESKGASSCR LLFCLLISAT VFRPGLGWYT VNSAYGDTII IPCRLDVPQN |
| SEQ ID NO: 14 | LMFGKWKYEK PDGSPVFIAF RSSTKKSVQY DDVPEYKDRL NLSENYTLSI |
| | SNARISDEKR FVCMLVTEDN VFEAPTIVKV FKQPSKPEIV SKALFLETEQ |
| | LKKLGDCISE DSYPDGNITW YRNGKVLHPL EGAVVIIFKK EMDPVTQLYT |
| | MTSTLEYKTT KADIQMPFTC SVTYYGPSGQ KTIHSEQAVF DIYYPTEQVT |
| | IQVLPPKNAI KEGDNITLKC LGNGNPPPEE FLFYLPGQPE GIRSSNTYTL |
| | TDVRRNATGD YKCSLIDKKS MIASTAITVH YLDLSLNPSG EVTRQIGDAL |
| | PVSCTISASR NATVVWMKDN IRLRSSPSFS SLHYQDAGNY VCETALQEVE |
| | GLKKRESLTL IVEGKPQIKM TKKTDPSGLS KTIICHVEGF PKPAIQWTIT |
| | GSGSVINQTE ESPYINGRYY SKIIISPEEN VTLTCTAENQ LERTVNSLNV SAISIPEHDE |
| | ADEISDENRE KVNDQAKLIV GIVVGLLLAA LVAGVVYWLY MKKSKTASKH |
| | VNKDLGNMEE NKKLEENNHK TEA |
| CD171 | MVVALRYVWP LLLCSPCLLI QIPEEYEGHH VMEPPVITEQ SPRRLVVFPT |
| SEQ ID NO: 15 | DDISLKCEAS GKPEVQFRWT RDGVHFKPKE ELGVTVYQSP HSGSFTITGN |
| | NSNFAQRFQG IYRCFASNKL GTAMSHEIRL MAEGAPKWPK ETVKPVEVEE |
| | GESVVLPCNP PPSAEPLRIY WMNSKILHIK QDERVTMGQN GNLYFANVLT |
| | SDNHSDYICH AHFPGTRTII QKEPIDLRVK ATNSMIDRKP RLLFPTNSSS |
| | HLVALQGQPL VLECIAEGFP TPTIKWLRPS GPMPADRVTY QNHNKTLQLL |
| | KVGEEDDGEY RCLAENSLGS ARHAYYVTVE AAPYWLHKPQ SHLYGPGETA |
| | RLDCQVQGRP QPEVTWRING IPVEELAKDQ KYRIQRGALI LSNVQPSDTM |
| | VTQCEARNRH GLLLANAYIY VVQLPAKILT ADNQTYMAVQ GSTAYLLCKA |
| | FGAPVPSVQW LDEDGTTVLQ DERFFPYANG TLGIRDLQAN DTGRYFCLAA |
| | NDQNNVTIMA NLKVKDATQI TQGPRSTIEK KGSRVTFTCQ ASFDPSLQPS |
| | ITWRGDGRDL QELGDSDKYF IEDGRLVIHS LDYSDQGNYS CVASTELDVV |
| | ESRAQLLVVG SPGPVPRLVL SDLHLLTQSQ VRVSWSPAED HNAPIEKYDI |
| | EFEDKEMAPE KWYSLGKVPG NQTSTTLKLS PYVHYTFRVT AINKYGPGEP |
| | SPVSETVVTP EAAPEKNPVD VKGEGNETTN MVITWKPLRW MDWNAPQVQY |
| | RVQWRPQGTR GPWQEQIVSD PFLVVSNTST FVPYEIKVQA VNSQGKGPEP |
| | QVTIGYSGED YPQAIPELEG IEILNSSAVL VKWRPVDLAQ VKGHLRGYNV |
| | TYWREGSQRK HSKRHIHKDH VVVPANTTSV ILSGLRPYSS YHLEVQAFNG |
| | RGSGPASEFT FSTPEGVPGH PEALHLECQS NTSLLLRWQP PLSHNGVLTG |
| | YVLSYHPLDE GGKGQLSFNL RDPELRTHNL TDLSPHLRYR FQLQATTKEG |
| | PGEAIVREGG TMALSGISDF GNISATAGEN YSVVSWVPKE GQCNFRFHIL |
| | FKALGEEKGG ASLSPQYVSY NQSSYTQWDL QPDTDYEIHL FKERMFRHQM |
| | AVKTNGTGRV RLPPAGFATE GWFIGFVSAI ILLLLVLLIL CFIKRSKGGK |
| | YSVKDKEDTQ VDSEARPMKD ETFGEYRSLE SDNEEKAFGS SQPSLNGDIK |
| | PLGSDDSLAD YGGSVDVQFN EDGSFIGQYS GKKEKEAAGG NDSSGATSPI NPAVALE |
| NGFR | MGAGATGRAM DGPRLLLLLL LGVSLGGAKE ACPTGLYTHS GECCKACNLG |
| SEQ ID NO: 16 | EGVAQPCGAN QTVCEPCLDS VTFSDVVSAT EPCKPCTECV GLQSMSAPCV |
| | EADDAVCRCA YGYYQDETTG RCEACRVCEA GSGLVFSCQD KQNTVCEECP |
| | DGTYSDEANH VDPCLPCTVC EDTERQLREC TRWADAECEE IPGRWITRST |
| | PPEGSDSTAP STQEPEAPPE QDLIASTVAG VVTTVMGSSQ PVVTRGTTDN |
| | LIPVYCSILA AVVVGLVAYI AFKRWNSCKQ NKQGANSRPV NQTPPPEGEK |
| | LHSDSGISVD SQSLHDQQPH TQTASGQALK GDGGLYSSLP PAKREEVEKL |
| | LNGSAGDTWR HLAGELGYQP EHIDSFTHEA CPVRALLASW ATQDSATLDA |
| | LLAALRRIQR ADLVESLCSE STATSPV |
| SOX10 | MAEEQDLSEV ELSPVGSEEP RCLSPGSAPS LGPDGGGGGS GLRASPGPGE |
| SEQ ID NO: 17 | LGKVKKEQQD GEADDDKFPV CIREAVSQVL SGYDWTLVPM PVRVNGASKS |
| | KPHVKRPMNA FMVWAQAARR KLADQYPHLH NAELSKTLGK LWRLLNESDK |
| | RPFIEEAERL RMQHKKDHPD YKYQPRRRKN GKAAQGEAEC PGGEAEQGGT |
| | AAIQAHYKSA HLDHRHPGEG SPMSDGNPEH PSGQSHGPPT PPTTPKTELQ |
| | SGKADPKRDG RSMGEGGKPH IDFGNVDIGE ISHEVMSNME TFDVAELDQY |
| | LPPNGHPGHV SSYSAAGYGL GSALAVASGH SAWISKPPGV ALPTVSPPGV |
| | DAKAQVKTET AGPQGPPHYT DQPSTSQIAY TSLSLPHYGS AFPSISRPQF |
| | DYSDHQPSGP YYGHSGQASG LYSAFSYMGP SQRPLYTAIS DPSPSGPQSH |
| | SPTHWEQPVY TTLSRP |
| POU3F2 | MATAASNHYS LLTSSASIVH AEPPGGMQQG AGGYREAQSL VQGDYGALQS |
| SEQ ID NO: 18 | NGHPLSHAHQ WITALSHGGG GGGGGGGGGG GGGGGGGGDG SPWSTSPLGQ |
| | PDIKPSVVVQ QGGRGDELHG PGALQQQHQQ QQQQQQQQQQ QQQQQQQQQR |
| | PPHLVHHAAN HHPGPGAWRS AAAAAHLPPS MGASNGGLLY SQPSFTVNGM |
| | LGAGGQPAGL HHHGLRDAHD EPHHADHHPH PHSHPHQQPP PPPPPQGPPG |
| | HPGAHHDPHS DEDTPTSDDL EQFAKQFKQR RIKLGFTQAD VGLALGTLYG |
| | NVFSQTTICR FEALQLSFKN MCKLKPLLNK WLEEADSSSG SPTSIDKIAA |
| | QGRKRKKRTS IEVSVKGALE SHFLKCPKPS AQEITSLADS LQLEKEVVRV |
| | WFCNRRQKEK RMTPPGGTLP GAEDVYGGSR DTPPHHGVQT PVQ |
| MPZ | MAPGAPSSSP SPILAVLLFS SLVLSPAQAI VVYTDREVHG AVGSRVTLHC |
| SEQ ID NO: 19 | SFWSSEWVSD DISFTWRYQP EGGRDAISIF HYAKGQPYID EVGTFKERIQ |
| | WVGDPRWKDG SIVIHNLDYS DNGTFTCDVK NPPDIVGKTS QVTLYVFEKV |
| | PTRYGVVLGA VIGGVLGVVL LLLLLFYVVR YCWLRRQAAL QRRLSAMEKG |
| | KLHKPGKDAS KRGRQTPVLY AMLDHSRSTK AVSEKKAKGL GESRKDKKXR |
| | LAGRAGDRGL GVESAKGPKV MVIEMELRKD EQSPELRPAV KSPSRTSLKN |
| | ALKNMMGLNS DK |
| GAP43 | MTKSCSELCH PALHELPCLG GLRKNLQRAV RPSPYSLGFL TFWISRVEKN |
| SEQ ID NO: 20 | DDDQKIEQDG IKPEDKAHKA ATKIQASFRG HITRKKLKGE KKDDVQAAEA |
| | EANKKDEAPV ADGVEKKGEG TTTAEAAPAT GSKPDEPGKA GETPSEEKKG |
| | EGDAATEQAA PQAPASSEEK AGSAETESAT KASTDNSPSS KAEDAPAKEE |
| | PKQADVPAAV TAAAATTPAA EDAAAKATAQ PPTETGESSQ AEENIEAVDE |
| | TKPKESARQD EGKEEEPEAD QEHA |
| ERBB3 | MRANDALQVL GLLFSLARGS EVGNSQAVCP GTLNGLSVTG DAENQYQTLY |
| SEQ ID NO: 21 | KLYERCEVVM GNLEIVLTGH NADLSFLQWI REVTGYVLVA MNEFSTLPLP |
| | NLRVVRGTQV YDGKFAIFVM LNYNTNSSHA LRQLRLTQLT EILSGGVYIE |
| | KNDKLCHMDT IDWRDIVRDR DAEIVVKDNG RSCPPCHEVC KGRCWGPGSE |
| | DCQTLTKTIC APQCNGHCFG PNPNQCCHDE CAGGCSGPQD TDCFACRHEN |
| | DSGACVPRCP QPLVYNKLTF QLEPNPHTKY QYGGVCVASC PHNFVVDQTS |
| | CVRACPPDKM EVDKNGLKMC EPCGGLCPKA CEGTGSGSRF QTVDSSNIDG |
| | FVNCTKILGN LDFLITGLNG DPWHKIPALD PEKLNVERTV REITGYLNIQ |
| | SWPPHMHNFS VESNLTTIGG RSLYNRGFSL LIMKNLNVTS LGFRSLKEIS |
| | AGRIYISANR QLCYHHSLNW TKVLRGPTEE RLDIKHNRPR RDCVAEGKVC |
| | DPLCSSGGCW GPGPGQCLSC RNYSRGGVCV THCNFLNGEP REFAHEAECF |
| | SCHPECQPME GTATCNGSGS DTCAQCAHFR DGPHCVSSCP HGVLGAKGPI |
| | YKYPDVQNEC RPCHENCTQG CKGPELQDCL GQTLVLIGKT HLTMALTVIA |
| | GLVVIFMMLG GTFLYWRGRR IQNKRAMRRY LERGESIEPL DPSEKANKVL |
| | ARIFKETELR KLKVLGSGVF GTVHKGVWIP EGESIKIPVC IKVIEDKSGR |
| | QSFQAVTDHM LAIGSLDHAH IVRLLGLCPG SSLQLVTQYL PLGSLLDHVR |
| | QHRGALGPQL LLNWGVQIAK GMYYLEEHGM VHRNLAARNV LLKSPSQVQV |
| | ADFGVADLLP PDDKQLLYSE AKTPIKWMAL ESIHFGKYTH QSDVWSYGVT |
| | VWELMTFGAE PYAGLRLAEV PDLLEKGERL AQPQICTIDV YMVMVKCWMI |
| | DENIRPTFKE LANEFTRMAR DPPRYLVIKR ESGPGIAPGP EPHGLTNKKL |
| | EEVELEPELD LDLDLEAEED NLATTTLGSA LSLPVGTLNR PRGSQSLLSP |
| | SSGYMPMNQG NLGESCQESA VSGSSERCPR PVSLHPMPRG CLASESSEGH |
| | VTGSEAELQE KVSMCRSRSR SRSPRPRGDS AYHSQRHSLL TPVTPLSPPG |
| | LEEEDVNGYV MPDTHLKGTP SSREGTLSSV GLSSVLGTEE EDEDEEYEYM |
| | NRRRRHSPPH PPRPSSLEEL GYEYMDVGSD LSASLGSTQS CPLHPVPIMP |
| | TAGTTPDEDY EYMNRQRDGG GPGGDYAAMG ACPASEQGYE EMRAFQGPGH |
| | QAPHVHYARL KTLRSLEATD SAFDNPDYWH SRLFPKANAQ RT |
| GDNF | MQSLPNSNGA AAGRDFKMKL WDVVAVCLVL LHTASAFPLP AGKRPPEAPA |
| SEQ ID NO: 22 | EDRSLGRRRA PFALSSDSNM PEDYPDQFDD VMDFIQATIK RLKRSPDKQM |
| | AVLPRRERNR QAAAANPENS RGKGRRGQRG KNRGCVLTAI HLNVTDLGLG |
| | YETKEELIFR YCSGSCDAAE TTYDKILKNL SRNRRLVSDK VGQACCRPIA |
| | FDDDLSFLDD NLVYHILRKH SAKRCGCI |
| MAG | MIFLTALPLF WIMISASRGG HWGAWMPSSI SAFEGTCVSI PCREDFPDEL |
| SEQ ID NO: 23 | RPAVVHGVWY FNSPYPKNYP PVVFKSRTQV VHESFQGRSR LLGDLGLRNC |
| | TLLLSNVSPE LGGKYYFRGD LGGYNQYTES EHSVLDIVNT PNIVVPPEVV |
| | AGTEVEVSCM VPDNCPELRP ELSWLGHEGL GEPAVLGRLR EDEGTWVQVS |
| | LLHFVPTREA NGHRLGCQAS FPNTTLQFEG YASMDVKYPP VIVEMNSSVE |
| | AIEGSHVSLL CGADSNPPPL LTWMRDGTVL REAVAESLLL ELEEVTPAED |
| | GVYACLAENA YGQDNRTVGL SVMYAPWKPT VNGTMVAVEG ETVSILCSTQ |
| | SNPDPILTIF KEKQILSTVI YESELQLELP AVSPEDDGEY WCVAENQYGQ |
| | RATAFNLSVE FAPVLLLESH CAAARDTVQC LCVVKSNPEP SVAFELPSRN |
| | VTVNESEREF VYSERSGLVL TSILTLRGQA QAPPRVICTA RNLYGAKSLE |
| | LPFQGAHRLM WAKIGPVGAV VAFAILIAIV CYITQTRRKK NVTESPSFSA |
| | GDNPPVLFSS DFRISGAPEK YESERRLGSE RRLLGLRGEP PELDLSYSHS |
| | DLGKRPTKDS YTLTEELAEY AEIRVK |
| PLLP | MAEFPSKVST RTSSPAQGAE ASVSALRPDL GFVRSRLGAL MLLQLVLGLL |
| SEQ ID NO: 24 | VWALIADTPY HLYPAYGWVM FVAVFLWLVT IVLFNLYLFQ LHMKLYMVPW |
| | PLVLMIFNIS ATVLYITAFI ACSAAVDLTS LRGTRPYNQR AAASFFACLV |
| | MIAYGVSAFF SYQAWRGVGS NAATSQMAGG YA |
| POU6F2 | MHARNPHSPF QRQHMMDCYL SAQQDTGTMQ AVIGQDPMIA GQVSKPLLSV |
| SEQ ID NO: 25 | RSEMNAELRG EDKAATSDSE LNEPLLAPVE SNDSEDTPSK LFGARGNPAL |
| | SDPGTPDQHQ ASQTHPPFPV GPQPLLTAQQ LASAVAGVMP GGPPALNQPI |
| | LIPFNMAGQL GGQQGLVLTL PTANLTNIQG LVAAAAAGGI MTLPLQNLQA |
| | TSSLNSQLQQ LQLQLQQQQQ QQQQQPPPST NQHPQPAPQA PSQSQQQPLQ |
| | PTPPQQPPPA SQQPPAPTSQ LQQAPQPQQH QPHSHSQNQN QPSPTQQSSS |
| | PPQKPSQSPG HGLPSPLTPP NPLQLVNNPL ASQAAAAAAA MSSIASSQAF |
| | GNALSSLQGV TGQLVTNAQG QIIGTIPLMP NPGPSSQAAS GTQGLQVQPI |
| | TPQLLTNAQG QIIATVIGNQ ILPVINTQGI TLSPIKPGQQ LHQPSQTSVG |
| | QAASQGNLLH LAHSQASMSQ SPVRQASSSS SSSSSSSALS VGQLVSNPQT |
| | AAGEVDGVNL EEIREFAKAF KIRRLSLGLT QTQVGQALSA TEGPAYSQSA |
| | ICRHTILRSH FFLPQEAQEN TIASSLTAKL NPGLLYPARF EKLDITPKSA |
| | QKIKPVLERW MAEAEARHRA GMQNLTEFIG SEPSKKRKRR TSFTPQALEI |
| | LNAHFEKNTH PSGQEMTEIA EKLNYDREVV RVWFCNKRQA LKNTIKRLKQ |
| | HEPATAVPLE PLTDSLEENS |
| PLXNB3 | MELTPASSLT CSLLSPRLPG SFPQLRRVPP CSRPWLPKAP VMARWPPFGL |
| SEQ ID NO: 26 | CLLLLLLSPP PLPLTGAHRF SAPNTTLNHL ALAPGRGTLY VGAVNRLFQL |
| | SPELQLEAVA VTGPVIDSPD CVPFRDPAEC PQAQLTDNAN QLLLVSSRAQ |
| | ELVACGQVRQ GVCETRRLGD VAEVLYQAED PGDGQFVAAN TPGVATVGLV |
| | VPLPGRDLLL VARGLAGKLS AGVPPLAIRQ LAGSQPFSSE GLGRLVVGDF |
| | SDYNNSYVGA FADARSAYFV FRRRGARAQA EYRSYVARVC LGDTNLYSYV |
| | EVPLACQGQG LIQAAFLAPG TLLGVFAAGP RGTQAALCAF PMVELGASME |
| | QARRLCYTAG GRGPSGAEEA TVEYGVTSRC VTLPLDSPES YPCGDEHTPS |
| | PIAGRQPLEV QPLLKLGQPV SAVAALQADG HMIAFLGDTQ GQLYKVFLHG |
| | SQGQVYHSQQ VGPPGSAISP DLLLDSSGSH LYVLTAHQVD RIPVAACPQF |
| | PDCASCLQAQ DPLCGWCVLQ GRCTRKGQCG RAGQLNQWLW SYEEDSHCLH |
| | IQSLLPGHHP RQEQGQVTLS VPRLPILDAD EYFHCAFGDY DSLAHVEGPH |
| | VACVTPPQDQ VPLNPPGTDH VTVPLALMFE DVTVAATNFS FYDCSAVQAL |
| | EAAAPCRACV GSIWRCHWCP QSSHCVYGEH CPEGERTIYS AQEVDIQVRG |
| | PGACPQVEGL AGPHLVPVGW ESHLALRVRN LQHFRGLPAS FHCWLELPGE |
| | LRGLPATLEE TAGDSGLIHC QAHQFYPSMS QRELPVPIYV TQGEAQRLDN |
| | THALYVILYD CAMGHPDCSH CQAANRSLGC LWCADGQPAC RYGPLCPPGA |
| | VELLCPAPSI DAVEPLTGPP EGGLALTILG SNLGRAFADV QYAVSVASRP |
| | CNPEPSLYRT SARIVCVTSP APNGTTGPVR VAIKSQPPGI SSQHFTYQDP |
| | VLLSLSPRWG PQAGGTQLTI RGQHLQTGGN TSAFVGGQPC PILEPVCPEA |
| | IVCRTRPQAA PGEAAVLVVF GHAQRTLLAS PFRYTANPQL VAAEPSASFR |
| | GGGRLIRVRG TGLDVVQRPL LSVWLEADAE VQASRAQPQD PQPRRSCGAP |
| | AADPQACIQL GGGLLQCSTV CSVNSSSLLL CRSPAVPDRA HPQRVFFTLD |
| | NVQVDFASAS GGQGFLYQPN PRLAPLSREG PARPYRLKPG HVLDVEGEGL |
| | NLGISKEEVR VHIGRGECLV KTLTRTHLYC EPPAHAPQPA NGSGLPQFVV |
| | QMGNVQLALG PVQYEAEPPL SAFPVEAQAG VGMGAAVLIA AVLLLTLMYR |
| | HKSKQALRDY QKVLVQLESL ETGVGDQCRK EFTDLMTEMT DLSSDLEGSG |
| | IPFLDYRTYA ERAFFPGHGG CPLQPKPEGP GEDGHCATVR QGLTQLSNLL |
| | NSKLFLLTLI HTLEEQPSFS QRDRCHVASL LSLALHGKLE YLTDIMRTLL |
| | GDLAAHYVHR NPKLMLRRTE TMVEKLLTNW LSICLYAFLR EVAGEPLYML |
| | FRAIQYQVDK GPVDAVTGKA KRTLNDSRLL REDVEFQPLT LMVLVGPGAG |
| | GAAGSSEMQR VPARVLDTDT ITQVKEKVLD QVYKGTPFSQ RPSVHALDLE |
| | WRSGLAGHLT LSDEDLTSVT QNHWKRLNTL QHYKVPDGAT VGLVPQLHRG |
| | STISQSLAQR CPLGENIPTL EDGEEGGVCL WHLVKATEEP EGAKVRCSSL |
| | REREPARAKA IPEIYLTRLL SMKGTLQKFV DDTFQAILSV NRPIPIAVKY |
| | LFDLLDELAE KHGIEDPGTL HIWKTNSLLL RFWVNALKNP QLIFDVRVSD |
| | NVDAILAVIA QTFIDSCTTS EHKVGRDSPV NKLLYAREIP RYKQMVERYY |
| | ADIRQSSPAS YQEMNSALAE LSGNYTSAPH CLEALQELYN HIHRYYDQII |
| | SALEEDPVGQ KLQLACRLQQ VAALVENKVT DL |

In some embodiments, the compositions of the present disclosure comprise greater than about 25% Schwann cells. In some embodiments, the compositions of the present disclosure comprise greater than about 30% Schwann cells. In some embodiments, the compositions of the present disclosure comprise greater than about 35% Schwann cells. In some embodiments, the compositions of the present disclosure comprise greater than about 40% Schwann cells. In some embodiments, the compositions of the present disclosure comprise greater than about 45% Schwann cells. In some embodiments, the compositions of the present disclosure comprise greater than about 50% Schwann cells. In some embodiments, the compositions of the present disclosure comprise greater than about 55% Schwann cells. In some embodiments, the compositions of the present disclosure comprise greater than about 60% Schwann cells. In some embodiments, the compositions of the present disclosure comprise greater than about 65% Schwann cells. In some embodiments, the compositions of the present disclosure comprise greater than about 70% Schwann cells. In some embodiments, the compositions comprise greater than about 75% Schwann cells. In some embodiments, the compositions comprise greater than about 80% Schwann cells. In some embodiments, the compositions comprise greater than about 85% Schwann cells. In some embodiments, the compositions comprise greater than about 90% Schwann cells. In some embodiments, the compositions comprise greater than about 95% Schwann cells. In some embodiments, the compositions comprise greater than about 99% Schwann cells.

In some embodiments, the compositions of the present disclosure comprise SCs derived from pluripotent stem cells. In some embodiments, the compositions of the present disclosure are passaged for at least about 1 week, at least about 2 weeks, or at least about 3 weeks, while maintaining expression of one or a combination of: CD98, S100, MBP, GFAP, PMP22 or functional fragments or variants thereof.

In some embodiments, the compositions of the present disclosure comprise a spheroid. A spheroid of the present invention can have any suitable width, length, thickness, and/or diameter. In some embodiments, a spheroid may have a width, length, thickness, and/or diameter in a range from about 100 μm to about 50,000 μm, or any range therein, such as, but not limited to, from about 100 μm to about 900 μm, from about 100 μm to about 700 μm, from about 300 μm to about 600 μm, from about 400 μm to about 500 μm, from about 500 μm to about 1,000 μm, from about 600 μm to about 1,000 μm, from about 700 μm to about 1,000 μm, from about 800 μm to about 1,000 μm, from about 900 μm to about 1,000 μm, from about 750 μm to about 1,500 μm, from about 1,000 μm to about 5,000 μm, from about 1,000 μm to about 10,000 μm, from about 2,000 to about 50,000 μm, from about 25,000 μm to about 40,000 μm, or from about 3,000 μm to about 15,000 μm. In some embodiments, a spheroid may have a width, length, thickness, and/or diameter of about 100 μm, 200 μm, 300 μm, 400 μm, 500 μm, 600 μm, 700 μm, 800 μm, 900 μm, 1,000 μm, 5,000 μm, 10,000 μm, 20,000 μm, 30,000 μm, 40,000 μm, or about 50,000 μm. In some embodiments, a plurality of spheroids are generated, and each of the spheroids of the plurality may have a width, length, thickness, and/or diameter that varies by less than about 20%, such as, for example, less than about 15%, 10%, or 5%. In some embodiments, each of the spheroids of the plurality may have a different width, length, thickness, and/or diameter within any of the ranges set forth above.

The cells in a spheroid may have a particular orientation. In some embodiments, the spheroid may comprise an interior core and an exterior surface. In some embodiments, the spheroid may be hollow (i.e., may not comprise cells in the interior). In some embodiments, the interior core cells and the exterior surface cells are different types of cell. In some embodiments, the spheroid comprises a neural crest cell and at least one Schwann cell.

In some embodiments, spheroids may be made up of one, two, three or more different cell types, including one or a plurality of neuronal cell types and/or one or a plurality of stem cell types. In some embodiments, the interior core cells may be made up of one, two, three, or more different cell types. In some embodiments, the exterior surface cells may be made up of one, two, three, or more different cell types. In some embodiments, the spheroids comprise a Schwann cell.

In some embodiments, the spheroids comprise at least two types of cells, wherein at least on cell type is a SC.

In some embodiments, the hydrogel or hydrogel matrixes can have various thicknesses. In some embodiments, the thickness of the hydrogel or hydrogel matrix is from about 100 μm to about 800 μm. In some embodiments, the thickness of the hydrogel or hydrogel matrix is from about 150 μm to about 800 μm. In some embodiments, the thickness of the hydrogel or hydrogel matrix is from about 200 μm to about 800 μm. In some embodiments, the thickness of the hydrogel or hydrogel matrix is from about 250 μm to about 800 μm. In some embodiments, the thickness of the hydrogel or hydrogel matrix is from about 300 μm to about 800 μm. In some embodiments, the thickness of the hydrogel or hydrogel matrix is from about 350 μm to about 800 μm. In some embodiments, the thickness of the hydrogel or hydrogel matrix is from about 400 μm to about 800 μm. In some embodiments, the thickness of the hydrogel or hydrogel matrix is from about 450 μm to about 800 μm. In some embodiments, the thickness of the hydrogel or hydrogel matrix is from about 500 μm to about 800 μm. In some embodiments, the thickness of the hydrogel or hydrogel matrix is from about 550 μm to about 800 μm. In some embodiments, the thickness of the hydrogel or hydrogel matrix is from about 600 μm to about 800 μm. In some embodiments, the thickness of the hydrogel or hydrogel matrix is from about 650 μm to about 800 μm. In some embodiments, the thickness of the hydrogel or hydrogel matrix is from about 700 μm to about 800 μm. In some embodiments, the thickness of the hydrogel or hydrogel matrix is from about 750 μm to about 800 μm. In some embodiments, the thickness of the hydrogel or hydrogel matrix is from about 100 μm to about 750 μm. In some embodiments, the thickness of the hydrogel or hydrogel matrix is from about 100 μm to about 700 μm. In some embodiments, the thickness of the hydrogel or hydrogel matrix is from about 100 μm to about 650 μm. In some embodiments, the thickness of the hydrogel or hydrogel matrix is from about 100 μm to about 600 μm. In some embodiments, the thickness of the hydrogel or hydrogel matrix is from about 100 μm to about 550 μm. In some embodiments, the thickness of the hydrogel or hydrogel matrix is from about 100 μm to about 500 μm. In some embodiments, the thickness of the hydrogel or hydrogel matrix is from about 100 μm to about 450 μm. In some embodiments, the thickness of the hydrogel or hydrogel matrix is from about 100 μm to about 400 μm. In some embodiments, the thickness of the hydrogel or hydrogel matrix is from about 100 μm to about 350 μm. In some embodiments, the thickness of the hydrogel or hydrogel matrix is from about 100 μm to about 300 μm. In some embodiments, the thickness of the hydrogel or hydrogel matrix is from about 100 μm to about 250 μm. In some embodiments, the thickness of the hydrogel or hydrogel matrix is from about 100 μm to about 200 μm. In some embodiments, the thickness of the hydrogel or hydrogel matrix is from about 100 μm to about 150 μm. In some embodiments, the thickness of the hydrogel or hydrogel matrix is from about 300 μm to about 600 μm. In some embodiments, the thickness of the hydrogel or hydrogel matrix is from about 400 μm to about 500 μm.

In some embodiments, the hydrogel or hydrogel matrixes can have various thicknesses. In some embodiments, the thickness of the hydrogel or hydrogel matrix is from about 10 μm to about 3000 μm. In some embodiments, the thickness of the hydrogel or hydrogel matrix is from about 150 μm to about 3000 μm. In some embodiments, the thickness of the hydrogel or hydrogel matrix is from about 200 μm to about 3000 μm. In some embodiments, the thickness of the hydrogel or hydrogel matrix is from about 250 μm to about 3000 μm. In some embodiments, the thickness of the hydrogel or hydrogel matrix is from about 300 μm to about 3000 μm. In some embodiments, the thickness of the hydrogel or hydrogel matrix is from about 350 μm to about 3000 μm. In some embodiments, the thickness of the hydrogel or hydrogel matrix is from about 400 μm to about 3000 μm. In some embodiments, the thickness of the hydrogel or hydrogel matrix is from about 450 μm to about 3000 μm. In some embodiments, the thickness of the hydrogel or hydrogel matrix is from about 500 μm to about 3000 μm. In some embodiments, the thickness of the hydrogel or hydrogel matrix is from about 550 μm to about 3000 μm. In some embodiments, the thickness of the hydrogel or hydrogel matrix is from about 600 μm to about 3000 μm. In some embodiments, the thickness of the hydrogel or hydrogel matrix is from about 650 μm to about 3000 μm. In some embodiments, the thickness of the hydrogel or hydrogel matrix is from about 700 μm to about 3000 μm. In some embodiments, the thickness of the hydrogel or hydrogel matrix is from about 750 μm to about 3000 μm. In some embodiments, the thickness of the hydrogel or hydrogel matrix is from about 800 μm to about 3000 μm. In some embodiments, the thickness of the hydrogel or hydrogel matrix is from about 850 μm to about 3000 μm. In some embodiments, the thickness of the hydrogel or hydrogel matrix is from about 900 μm to about 3000 μm. In some embodiments, the thickness of the hydrogel or hydrogel matrix is from about 950 μm to about 3000 μm. In some embodiments, the thickness of the hydrogel or hydrogel matrix is from about 1000 μm to about 3000 μm. In some embodiments, the thickness of the hydrogel or hydrogel matrix is from about 1500 μm to about 3000 μm. In some embodiments, the thickness of the hydrogel or hydrogel matrix is from about 2000 μm to about 3000 μm. In some embodiments, the thickness of the hydrogel or hydrogel matrix is from about 2500 μm to about 3000 μm. In some embodiments, the thickness of the hydrogel or hydrogel matrix is from about 100 μm to about 2500 μm. In some embodiments, the thickness of the hydrogel or hydrogel matrix is from about 100 μm to about 2000 μm. In some embodiments, the thickness of the hydrogel or hydrogel matrix is from about 100 μm to about 1500 μm. In some embodiments, the thickness of the hydrogel or hydrogel matrix is from about 100 μm to about 1000 μm. In some embodiments, the thickness of the hydrogel or hydrogel matrix is from about 100 μm to about 950 μm. In some embodiments, the thickness of the hydrogel or hydrogel matrix is from about 100 μm to about 900 μm. In some embodiments, the thickness of the hydrogel or hydrogel matrix is from about 100 μm to about 850 μm. In some embodiments, the thickness of the hydrogel or hydrogel matrix is from about 100 μm to about 800 μm. In some embodiments, the thickness of the hydrogel or hydrogel matrix is from about 100 μm to about 750 μm. In some embodiments, the thickness of the hydrogel or hydrogel matrix is from about 100 μm to about 700 μm. In some embodiments, the thickness of the hydrogel or hydrogel matrix is from about 100 μm to about 650 μm. In some embodiments, the thickness of the hydrogel or hydrogel matrix is from about 100 μm to about 600 μm. In some embodiments, the thickness of the hydrogel or hydrogel matrix is from about 100 μm to about 550 μm. In some embodiments, the thickness of the hydrogel or hydrogel matrix is from about 100 μm to about 500 μm. In some embodiments, the thickness of the hydrogel or hydrogel matrix is from about 100 μm to about 450 μm. In some embodiments, the thickness of the hydrogel or hydrogel matrix is from about 100 μm to about 400 μm. In some embodiments, the thickness of the hydrogel or hydrogel matrix is from about 100 μm to about 350 μm. In some embodiments, the thickness of the hydrogel or hydrogel matrix is from about 100 μm to about 300 μm. In some embodiments, the thickness of the hydrogel or hydrogel matrix is from about 100 μm to about 250 μm. In some embodiments, the thickness of the hydrogel or hydrogel matrix is from about 100 μm to about 200 μm. In some embodiments, the thickness of the hydrogel or hydrogel matrix is from about 100 μm to about 150 μm. In some embodiments, the thickness of the hydrogel or hydrogel matrix is from about 300 μm to about 600 μm. In some embodiments, the thickness of the hydrogel or hydrogel matrix is from about 400 μm to about 500 μm.

In some embodiments, the hydrogel or hydrogel matrix comprises one or more synthetic polymers. In some embodiments, the hydrogel or hydrogel matrix comprises one or more of the following synthetic polymers: polyethylene glycol (polyethylene oxide), polyvinyl alcohol, poly-2-hydroxyethyl methacrylate, polyacrylamide, silicones, and any derivatives or combinations thereof.

In some embodiments, the hydrogel or hydrogel matrix comprises one or more synthetic and/or natural polysaccharides. In some embodiments, the hydrogel or hydrogel matrix comprises one or more of the following polysaccharides: hyaluronic acid, heparin sulfate, heparin, dextran, agarose, chitosan, alginate, and any derivatives or combinations thereof.

In some embodiments, the hydrogel or hydrogel matrix comprises one or more proteins and/or glycoproteins. In some embodiments, the hydrogel or hydrogel matrix comprises one or more of the following proteins: collagen, gelatin, elastin, titin, laminin, fibronectin, fibrin, keratin, polyornithine, silk fibroin, and any derivatives or combinations thereof.

In some embodiments, the one or plurality of cells is stimulated by a differentiation factor, or differentiator. Differentiation factors may include one or a combination of any of the following:

```
BMP4
[SEQ ID NO: 59]
MIPGNRMLMV VLLCQVLLGG ASHASLIPET GKKKVAEIQG HAGGRRSGQS
HELLRDFEAT LLQMFGLRRR PQPSKSAVIP DYMRDLYRLQ SGEEEEEQIH
STGLEYPERP ASRANTVRSF HHEEHLENIP GTSENSAFRF LFNLSSIPEN
EVISSAELRL FREQVDQGPD WERGFHRINI YEVMKPPAEV VPGHLITRLL
DTRLVHHNVT RWETFDVSPA VLRWTREKQP NYGLAIEVTH LHQTRTHQGQ
HVRISRSLPQ GSGNWAQLRP LLVTFGHDGR GHALTRRRRA KRSPKHHSQR
ARKKNKNCRR HSLYVDFSDV GWNDWIVAPP GYQAFYCHGD CPFPLADHLN
STNHAIVQTL VNSVNSSIPK ACCVPTELSA ISMLYLDEYD KVVLKNYQEM
VVEGCGCR
FGF2
  [SEQ ID NO: 60]
MVGVGGGDVE DVTPRPGGCQ ISGRGARGCN GIPGAAAWEA ALPRRRPRRH
PSVNPRSRAA GSPRTRGRRT EERPSGSRLG DRGRGRALPG GRLGGRGRGR
APERVGGRGR GRGTAAPRAA PAARGSRPGP AGTMAAGSIT TLPALPEDGG
SGAFPPGHFK DPKRLYCKNG GFFLRIHPDG RVDGVREKSD PHIKLQLQAE
ERGVVSIKGV CANRYLAMKE DGRLLASKCV TDECFFFERL ESNNYNTYRS
RKYTSWYVAL KRTGQYKLGS KTGPGQKAIL FLPMSAKS
NRG1
  [SEQ ID NO: 61]
MEIYSPDMSE VAAERSSSPS TQLSADPSLD GLPAAEDMPE PQTEDGRTPG
LVGLAVPCCA CLEAERLRGC LNSEKICIVP ILACLVSLCL CIAGLKWVFV
DKIFEYDSPT HLDPGGLGQD PIISLDATAA SAVWVSSEAY TSPVSRAQSE
SEVQVTVQGD KAVVSFEPSA APTPKNRIFA FSFLPSTAPS FPSPTRNPEV
RTPKSATQPQ TTETNLQTAP KLSTSTSTTG TSHLVKCAEK EKTFCVNGGE
CFMVKDLSNP SRYLCKCPNE FTGDRCQNYV MASFYSTSTP FLSLPE
```

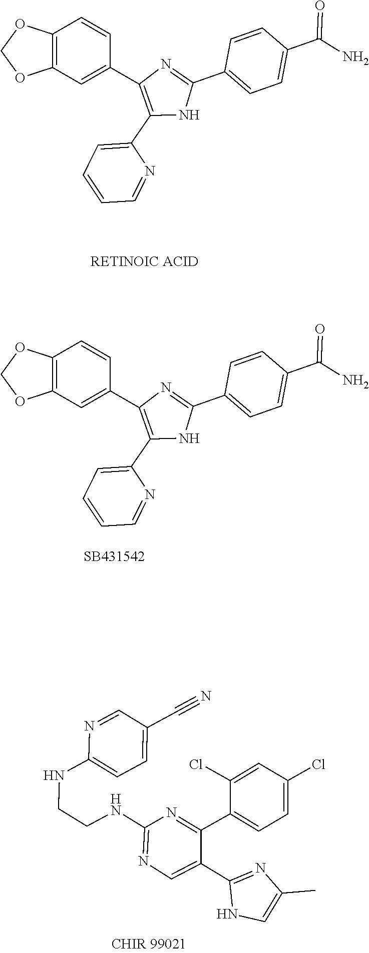 [SMILES: NC(=O)c1ccc(-c2nc(-c3ccc4c(c3)OCO4)c(-c3ccccn3)[nH]2)cc1 ; NC(=O)c1ccc(-c2nc(-c3ccc4c(c3)OCO4)c(-c3ccccn3)[nH]2)cc1 ; Cc1c[nH]c(-c2cnc(NCCNc3ccc(C#N)cn3)nc2-c2ccc(Cl)cc2Cl)n1]

The systems or methods disclosed herein can comprise a tissue culture medium comprising any one or combination of differentiation factors, or functional fragments, salts or derivatives thereof. In any of the methods or systems disclosed herein, the differentiation factors used may be functional fragments or variants of the polypeptides disclosed above with at least about 70% sequence identity to the above sequences. In any of the methods or systems disclosed herein, the differentiation factors used may be functional fragments or variants of the polypeptides disclosed above with at least about 80% sequence identity to the above sequences. In any of the methods or systems disclosed herein, the differentiation factors used may be functional fragments or variants of the polypeptides disclosed above with at least about 85% sequence identity to the above sequences. In any of the methods or systems disclosed herein, the differentiation factors used may be functional fragments or variants of the polypeptides disclosed above with at least about 90% sequence identity to the above sequences. In any of the methods or systems disclosed herein, the differentiation factors used may be functional fragments or variants of the polypeptides disclosed above with at least about 95% sequence identity to the above sequences. In any of the methods or systems disclosed herein, the differentiation factors used may be functional analogues of the small molecules disclosed above. The methods of the disclosure relate to the sequential exposure of a culture of cells to two or more different tissue culture mediums. In some embodiments, systems disclosed herein comprise one or more of the above-identified differentiation factors or functional fragments or derivatives thereof. In some embodiments, cells disclosed herein are exposed to an amount of a differentiation factor for a time period sufficient to differentiate the cell or cells into another cell type, such as SCs. In some embodiments, methods disclosed herein comprise exposing neural crest cells to one or combination of differentiation factors, or a salt thereof, for a time period sufficient to differentiate the neural crest cell into a SC. In such methods, the exposing step may be about 2, 3, 4, 5, 6, 7, 8, 9, 10, 11, or 12 or more days. In some embodiments, the methods of the disclosure comprise the step of exposing the Schwann cells to a segment of gastrointestinal tissue in culture.

### Pharmaceutical Compositions

In some embodiments, the compositions described above are pharmaceutical compositions.

In some embodiments, the composition, spheroid or pharmaceutical composition comprising an SC is administered at a desired dosage, which in some aspects includes a desired dose or number of cells and/or a desired ratio of neuronal cell subpopulations. In some embodiments, the dosage of cells is based on a total number of cells (or number per m2 body surface area or per kg body weight) and a desired ratio of the individual populations or sub-types. In some embodiments, the dosage of cells is based on a desired total number (or number per m2 body surface area or per kg of body weight) of cells in the individual populations or of individual cell types. In some embodiments, the dosage is based on a combination of such features, such as a desired number of total cells, desired ratio, and desired total number of cells in the individual populations.

In some embodiments, the composition, spheroid or pharmaceutical composition comprising an SC is administered at or within a tolerated difference of a desired dose of total cells, such as a desired dose of subtypes of neuronal cells, e.g., Schwann cells. In some aspects, the desired dose is a desired number of cells, a desired number of cells per unit of body surface area or a desired number of cells per unit of body weight of the subject to whom the cells are administered, e.g., cells/m2 or cells/kg. In some aspects, the desired dose is at or above a minimum number of cells or minimum number of cells per unit of body surface area or body weight. In some aspects, among the total cells, administered at the desired dose, the individual populations or sub-types are present at or near a desired output ratio as described herein, e.g., within a certain tolerated difference or error of such a ratio.

In some embodiments, the cells are administered at or within a tolerated difference of a desired dose. In some aspects, the desired dose is a desired number of cells, or a desired number of such cells per unit of body surface area or body weight of the subject to whom the cells are administered, e.g., cells/m² or cells/kg. In some aspects, the desired dose is at or above a minimum number of cells of the population, or minimum number of cells of the population per unit of body surface area or body weight.

Thus, in some embodiments, the dosage is based on a desired fixed dose of total cells and a desired ratio, and/or based on a desired fixed dose of two or more, e.g., each, of the individual neuronal subpopulations. Thus, in some embodiments, the dosage is based on a desired fixed or minimum dose of neuronal subpopulations and a desired ratio thereof.

In certain embodiments, the composition, spheroid or pharmaceutical composition comprising an SC is administered to the subject at a range of from about one million to about 100 billion cells, such as, e.g., from about 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as from about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases from about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual neuronal subpopulations of cells is within a range of between at or about 10⁴ and at or about 10⁹ cells/meter2 (m2) body surface area, such as between 10⁵ and 10⁶ cells/m2 body surface area, for example, at or about 1×10⁵ cells/m2, 1.5×10⁵ cells/m2, 2×10⁵ cells/m2, or 1×10⁶ cells/m2 body surface area. For example, in some embodiments, the cells are administered at, or within a certain range of error of from about 10⁴ and at or about 10⁹ neuronal cells/meter² (m²) body surface area, such as between 10⁵ and 10⁶ SCs/m² body surface area, for example, at or about 1×10⁵ SC cells/m², 1.5×10⁵ SC cells/m², 2×10⁵ SC cells/m², or 1×10⁶ SC cells/m² body surface area.

In some embodiments, the cells are administered at or within a certain range of error of between at or about 10⁴ and at or about 10⁹ cells/meter² (m2) body weight, such as between 10⁵ and 10⁶ cells/m² body weight, for example, at or about 1×10⁵ cells/m², 1.5×10⁵ cells/m², 2×10⁵ cells/kg, or 1×10⁶ cells/m² body surface area.

Pharmaceutical compositions provided by the present disclosure include compositions wherein the active ingredient (e.g., cells described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of cells effective to achieve the desired result, e.g., modulating the activity of a subject (e.g., increase the number of Schwann cells in the subject), and/or reducing, eliminating, or slowing the progression of disease symptoms (e.g., symptoms of peripheral neuropathy). Determination of a therapeutically effective amount of a compound of the disclosure is well within the capabilities of those skilled in the art, especially in light of the detailed disclosure herein.

The pharmaceutical composition may be formulated according to the mode of administration to be used. An injectable pharmaceutical composition may be sterile, pyrogen-free and particulate free. An isotonic formulation or solution may be used as a pharmaceutically acceptable carrier. Additives for isotonicity may include sodium chloride, dextrose, mannitol, sorbitol, and lactose. The isotonic solutions may include phosphate buffered saline. The pharmaceutical composition may further comprise stabilizers including gelatin and albumin. The stabilizing may allow the formulation to be stable at room or ambient temperature for extended periods of time such as LGS or polycations or polyanions to the pharmaceutical composition formulation.

### Systems

The present disclosure also relates to a system comprising: (i) a cell culture vessel optionally comprising a hydrogel; (ii) one or a plurality of stem cells or Schwann cells either in suspension or adhered to a solid substrate; and (iii) one or a plurality of differentiation factors.

The present disclosure also relates to a system comprising: (i) a cell culture vessel optionally comprising a hydrogel; (ii) one or a plurality of stem cells or Schwann cells either in suspension or as a component of a spheroid; and (iii) on or plurality of differentiation factors. In some embodiments, the system further comprises one or combination of culture mediums disclosed herein. The disclosure also relates to a method of culturing Schwann cells in a system, the system comprising: (i) a cell culture vessel optionally comprising a hydrogel; (ii) one or a plurality of stem cells or neural crest cells either in suspension or as a component of a spheroid; and (iii) one or plurality of differentiation factors. In some embodiments, the system further comprises one or combination of culture mediums disclosed herein. In some embodiments, the methods relate to replacing medium during a culture time of from about 12 to about 21 days at least one time to (i) expose one or a plurality of stem cells to a first cell medium for a time period sufficient to differentiate the one or plurality of stem cells into neural crest cells and the sequentially replacing the medium to (ii) expose one or plurality of neural crest cells to a second cell medium for a time period sufficient to differentiate the one or plurality of neural crest cells into Schwann cells.

In some embodiments, the system comprises a solid substrate. The term “solid substrate” as used herein refers to any substance that is a solid support that is free of or substantially free of cellular toxins. In some embodiments, the solid substrate comprise one or a combination of silica, plastic, and metal. In some embodiments, the solid substrate comprises pores of a size and shape sufficient to allow diffusion or non-active transport of proteins, nutrients, and gas through the solid substrate in the presence of a cell culture medium. In some embodiments, the pore size is no more than about 10, 9, 8, 7, 6, 5, 4, 3, 2 microns or 1 micron in diameter. One of ordinary skill could determine how big of a pore size is necessary based upon the contents of the cell culture medium and exposure of cells growing on the solid substrate in a particular microenvironment. For instance, one of ordinary skill in the art can observe whether any cultured cells in the system or device are viable under conditions with a solid substrate comprises pores of various diameters. In some embodiments, the solid substrate comprises a base with a predetermined shape that defines the shape of the exterior and interior surface. In some embodiments, the base comprises one or a combination of silica, plastic, ceramic, or metal and wherein the base is in a shape of a cylinder or in a shape substantially similar to a cylinder, such that a first polymer coats the interior surface of the base and define a cylindrical or substantially cylindrical interior chamber; and wherein the opening is positioned at one end of the cylinder. In some embodiments, the base comprises one or a plurality of pores of a size and shape sufficient to allow diffusion of protein, nutrients, and oxygen through the solid substrate in the presence of the cell culture medium. In some embodiments, the solid substrate comprises a plastic base with a pore size of no more than about 1 micron in diameter and comprises at least one layer of hydrogel matrix wherein the solid substrate comprises at least one compartment defined at least in part by the shape of an interior surface of the solid substrate and accessible from a point outside of the solid substrate by an opening, optionally positioned at one end of the solid substrate. In embodiments, where the solid substrate comprises a hollow interior portion defined by at least one interior surface, the cells in suspension or tissue explants may be seeded by placement of cells at or proximate to the opening such that the cells may adhere to at least a portion the interior surface of the solid substrate for prior to growth. The at least one compartment or hollow interior of the solid substrate allows a containment of the cells in a particular three-dimensional shape defined by the shape of the interior surface. In some embodiments, the solid substrate and encourages directional growth of the cells away from the opening. In the case of neuronal cells, the degree of containment and shape of the at least one compartment are conducive to axon growth from soma positioned within the at least one compartment and at or proximate to the opening.

In some embodiments, the solid substrate is coated with polyornithine (“PO”), laminin (“LM”) and/or fibronectin (“FN”).

The present disclosure provides devices, methods, and systems involving production, maintenance, and physiological interrogation of neural cells in microengineered configurations designed to mimic native nerve tissue anatomy. It is another object of the disclosure to provide a medium to high-throughput assay of neurological function for the screening of pharmacological and/or toxicological properties of chemical and biological agents. In some embodiments, the agents are cells, such as any type of cell disclosed herein, or antibodies, such as antibodies that are used to treat clinical disease. In some embodiments, the agents are any drugs or agents that are used to treat human disease such that toxicities, effects or neuromodulation can be compared among a new agent which is a proposed mammalian treatment and existing treatments from human disease. In some embodiments, new agents for treatment of human disease are treatments for neurodegenerative disease and are compared to existing treatments for neurodegenerative disease.

Similarly, information gathered from imaging can determine quantitative metrics for the degree of cell toxicology and lends further insight into toxic and neuroprotective mechanisms of various agents or compounds of interest. In some embodiments, the at least one agent comprises a small chemical compound. In some embodiments, the at least one agent comprises at least one environmental or industrial pollutant. In some embodiments, the at least one agent comprises one or a combination of small chemical compounds chosen from: chemotherapeutics, analgesics, cardiovascular modulators, cholesterol, neuroprotectants, neuromodulators, immunomodulators, anti-inflammatories, and anti-microbial drugs.

In some embodiments, the at least one agent comprises one or a combination of chemotherapeutics chosen from: Actinomycin, Alitretinoin, All-trans retinoic acid, Azacitidine, Azathioprine, Bexarotene, Bleomycin, Bortezomib, Capecitabine, Carboplatin, Chlorambucil, Cisplatin, Cyclophosphamide, Cytarabine, Dacarbazine (DTIC), Daunorubicin, Docetaxel, Doxifluridine, Doxorubicin, Epirubicin, Epothilone, Erlotinib, Etoposide, Fluorouracil, Gefitinib, Gemcitabine, Hydroxyurea, Idarubicin, Imatinib, Irinotecan, Mechlorethamine, Melphalan, Mercaptopurine, Methotrexate, Mitoxantrone, Nitrosoureas, Oxaliplatin, Paclitaxel, Pemetrexed, Romidepsin, Tafluposide, Temozolomide (Oral dacarbazine), Teniposide, Tioguanine (formerly Thioguanine), Topotecan, Tretinoin, Vakrubicin, Vemurafenib, Vinblastine, Vincristine, Vindesine, Vinorelbine, Vismodegib, and Vorinostat. In some embodiments, the at least one agent comprises one or a combination of analgesics chosen from: Paracetoamol, Non-steroidal anti-inflammatory drugs (NSAIDs), COX-2 inhibitors, opioids, flupirtine, tricyclic antidepressants, carbamaxepine, gabapentin, and pregabalin.

In some embodiments, the at least one agent comprises one or a combination of cardiovascular modulators chosen from: nepicastat, cholesterol, niacin, scutellaria, prenylamine, dehydroepiandrosterone, monatepil, esketamine, niguldipine, asenapine, atomoxetine, flunarizine, milnacipran, mexiletine, amphetamine, sodium thiopental, flavonoid, bretylium, oxazepam, and honokiol.

In some embodiments, the at least one agent comprises one or a combination of neuroprotectants and/or neuromodulators chosen from: tryptamine, galanin receptor 2, phenylalanine, phenethylamine, N-methylphenethylamine, adenosine, kyptorphin, substance P, 3-methoxytyramine, catecholamine, dopamine, GABA, calcium, acetylcholine, epinephrine, norepinephrine, and serotonin. In some embodiments, the at least one agent comprises one or a combination of immunomodulators chosen from: clenolizimab, enoticumab, ligelizumab, simtuzumab, vatelizumab, parsatuzumab, Imgatuzumab, tregalizaumb, pateclizumab, namulumab, perakizumab, faralimomab, patritumab, atinumab, ublituximab, futuximab, and duligotumab.

In some embodiments, the at least one agent comprises one or a combination of anti-inflammatories chosen from: ibuprofen, aspirin, ketoprofen, sulindac, naproxen, etodolac, fenoprofen, diclofenac, flurbiprofen, ketorolac, piroxicam, indomethacin, mefenamic acid, meloxicam, nabumetone, oxaprozin, ketoprofen, famotidine, meclofenamate, tolmetin, and salsalate. In some embodiments, the at least one agent comprises one or a combination of anti-microbials chosen from: antibacterials, antifungals, antivirals, antiparasitics, heat, radiation, and ozone.

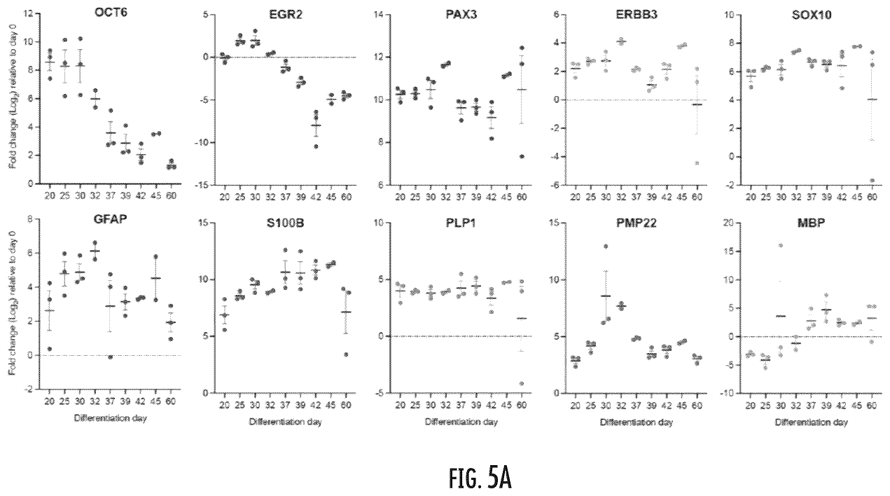

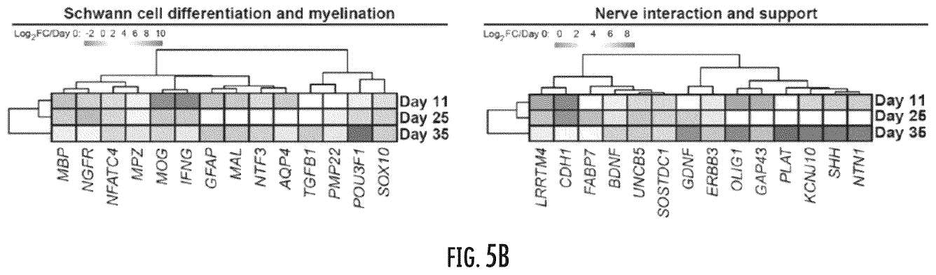

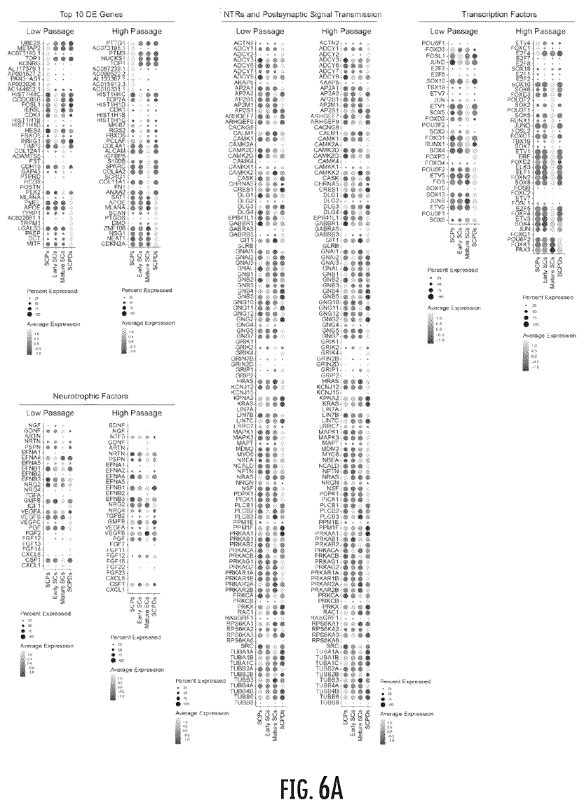

Table 2 is a list of those biomarkers specific for one or a plurality of cells disclosed in the application. Biomarkers may be expressed as proteins on the surface of the cells. In some embodiments, the biomarkers are expressed as mRNA encoding the proteins identified in Table 2 or functional fragments thereof. The biomarkers of the Figures, including FIG. 5, are disclosed in FIGS. 5E and 5F and matched with the cell type disclosed in those panels. It is understood that, if the cell type is matched with the gene name, then that cell type comprises a protein or expresses the nucleic acid sequence that is disclosed or encodes the amino acid associated with that nucleic acid sequence. Compositions of the disclosure relate to compositions comprising Low Passage Mature SCs, High Passage Mature SCs, Myelinating SCs and SCPDs. In some embodiments, compositions consist of one of: Low Passage Mature SCs, High Passage Mature SCs, Myelinating SCs and SCPDs. In some embodiments, compositions comprise one or a combination of: Low Passage Mature SCs, High Passage Mature SCs, Myelinating SCs and SCPDs. In some embodiments, the compositions comprise greater than about 20% of: Low Passage Mature SCs, High Passage Mature SCs, Myelinating SCs or SCPDs. In some embodiments, the compositions comprise greater than about 30% of: Low Passage Mature SCs, High Passage Mature SCs, Myelinating SCs or SCPDs. In some embodiments, the compositions comprise greater than about 40% of: Low Passage Mature SCs, High Passage Mature SCs, Myelinating SCs or SCPDs. In some embodiments, the compositions comprise greater than about 50% of: Low Passage Mature SCs, High Passage Mature SCs, Myelinating SCs or SCPDs. In some embodiments, the compositions comprise greater than about 60% of: Low Passage Mature SCs, High Passage Mature SCs, Myelinating SCs or SCPDs. In some embodiments, the compositions comprise greater than about 70% of: Low Passage Mature SCs, High Passage Mature SCs, Myelinating SCs or SCPDs. In some embodiments, the compositions comprise greater than about 80% of: Low Passage Mature SCs, High Passage Mature SCs, Myelinating SCs or SCPDs. In some embodiments, the compositions comprise greater than about 90% of: Low Passage Mature SCs, High Passage Mature SCs, Myelinating SCs or SCPDs.

In some embodiments, the cell types disclosed in the supplementary figures express RNA associated with the accession number in Table 3. In some embodiments, the cell types disclosed in the supplementary figures express protein associated with the accession number in Table 3.

TABLE 2

| Low Passage | High Passage | Myelinating | |
|---|---|---|---|
| Mature SC | mature SC | SC | SCPD |
| CD98 | CD98 | HLA-DR | POU6F2 |
| PMP22 | PMP22 | MAG | CD44 |
| NGFR | NGFR | MPZ | CD81 |
| SOX10 | ERBB3 | CNTN1 | |
| FOXO1 | GDNF | CNTN4 | |
| POU3F2 | NGF | CNTN6 | |
| TBX19 | BDNF | PLXNB3 | |
| MPZ | GAP43 | SECISBP2L | |
| MATN2 | SOX10 | CNP | |
| CD46 | FOXO1 | POU3F1 | |
| CD146 | POU3F2 | KIT | |
| CD147 | TBX19 | CRYAB | |
| CD166 | MPZ | UGT8 | |
| PLAT | MATN2 | PMP22 | |
| GAP43 | CD9 | PLLP | |
| ERBB3 | CD49e | | |
| GDNF | CD171 | | |
| | CD46 | | |
| | CD146 | | |
| | CD147 | | |
| | CD166 | | |
| | PLAT | | |
| | GAP43 | | |
| | ERBB3 | | |
| | GDNF | | |
| | NTN1 | | |

TABLE 3
Biomarker RNA sequences

| SEQ | | |
|---|---|---|
| ID | | |
| NO: | biomarker | RNA Sequence |
| 27 | CD98 | GCATTGCGGC TTGGTTTTCT CACCCAGTGC ATGTGGCAGG AGCGGTGAGA |
| | | TCACTGCCTC ACGGCGATCC TGGACTGACG GTCACGACTG CCTACCCTCT |
| | | AACCCTGTTC TGAGCTGCCC CTTGCCCACA CACCCCAAAC CTGTGTGCAG |
| | | GATCCGCCTC CATGGAGCTA CAGCCTCCTG AAGCCTCGAT CGCCGTCGTG |
| | | TCGATTCCGC GCCAGTTGCC TGGCTCACAT TCGGAGGCTG GTGTCCAGGG |
| | | TCTCAGCGCG GGGGACGACT CAGAGACGGG GTCTGACTGT GTTACCCAGG |
| | | CTGGTCTTCA ACTCTTGGCC TCAAGTGATC CTCCTGCCTT AGCTTCCAAG |
| | | AATGCTGAGG TTACAGTAGA AACGGGGTTT CACCATGTTA GCCAGGCTGA |
| | | TATTGAATTC CTGACCTCAA TTGATCCGAC TGCCTCGGCC TCCGGAAGTG |
| | | CTGGGATTAC AGGCACCATG AGCCAGGACA CCGAGGTGGA TATGAAGGAG |
| | | GTGGAGCTGA ATGAGTTAGA GCCCGAGAAG CAGCCGATGA ACGCGGCGTC |
| | | TGGGGCGGCC ATGTCCCTGG CGGGAGCCGA GAAGAATGGT CTGGTGAAGA |
| | | TCAAGGTGGC GGAAGACGAG GCGGAGGCGG CAGCCGCGGC TAAGTTCACG |
| | | GGCCTGTCCA AGGAGGAGCT GCTGAAGGTG GCAGGCAGCC CCGGCTGGGT |
| | | ACGCACCCGC TGGGCACTGC TGCTGCTCTT CTGGCTCGGC TGGCTCGGCA |
| | | TGCTTGCTGG TGCCGTGGTC ATAATCGTGC GAGCGCCGCG TTGTCGCGAG |
| | | CTACCGGCGC AGAAGTGGTG GCACACGGGC GCCCTCTACC GCATCGGCGA |
| | | CCTTCAGGCC TTCCAGGGCC ACGGCGCGGG CAACCTGGCG GGTCTGAAGG |
| | | GGCGTCTCGA TTACCTGAGC TCTCTGAAGG TGAAGGGCCT TGTGCTGGGT |
| | | CCAATTCACA AGAACCAGAA GGATGATGTC GCTCAGACTG ACTTGCTGCA |
| | | GATCGACCCC AATTTTGGCT CCAAGGAAGA TTTTGACAGT CTCTTGCAAT |
| | | CGGCTAAAAA AAAGAGCATC CGTGTCATTC TGGACCTTAC TCCCAACTAC |
| | | CGGGGTGAGA ACTCGTGGTT CTCCACTCAG GTTGACACTG TGGCCACCAA |
| | | GGTGAAGGAT GCTCTGGAGT TTTGGCTGCA AGCTGGCGTG GATGGGTTCC |
| | | AGGTTCGGGA CATAGAGAAT CTGAAGGATG CATCCTCATT CTTGGCTGAG |
| | | TGGCAAAATA TCACCAAGGG CTTCAGTGAA GACAGGCTCT TGATTGCGGG |
| | | GACTAACTCC TCCGACCTTC AGCAGATCCT GAGCCTACTC GAATCCAACA |
| | | AAGACTTGCT GTTGACTAGC TCATACCTGT CTGATTCTGG TTCTACTGGG |
| | | GAGCATACAA AATCCCTAGT CACACAGTAT TTGAATGCCA CTGGCAATCG |
| | | CTGGTGCAGC TGGAGTTTGT CTCAGGCAAG GCTCCTGACT TCCTTCTTGC |
| | | CGGCTCAACT TCTCCGACTC TACCAGCTGA TGCTCTTCAC CCTGCCAGGG |
| | | ACCCCTGTTT TCAGCTACGG GGATGAGATT GGCCTGGATG CAGCTGCCCT |
| | | TCCTGGACAG CCTATGGAGG CTCCAGTCAT GCTGTGGGAT GAGTCCAGCT |
| | | TCCCTGACAT CCCAGGGGCT GTAAGTGCCA ACATGACTGT GAAGGGCCAG |
| | | AGTGAAGACC CTGGCTCCCT CCTTTCCTTG TTCCGGCGGC TGAGTGACCA |
| | | GCGGAGTAAG GAGCGCTCCC TACTGCATGG GGACTTCCAC GCGTTCTCCG |
| | | CTGGGCCTGG ACTCTTCTCC TATATCCGCC ACTGGGACCA GAATGAGCGT |
| | | TTTCTGGTAG TGCTTAACTT TGGGGATGTG GGCCTCTCGG CTGGACTGCA |
| | | GGCCTCCGAC CTGCCTGCCA GCGCCAGCCT GCCAGCCAAG GCTGACCTCC |
| | | TGCTCAGCAC CCAGCCAGGC CGTGAGGAGG GCTCCCCTCT TGAGCTGGAA |
| | | CGCCTGAAAC TGGAGCCTCA CGAAGGGCTG CTGCTCCGCT TCCCCTACGC |
| | | GGCCTGACTT CAGCCTGACA TGGACCCACT ACCCTTCTCC TTTCCTTCCC |
| | | AGGCCCTTTG GCTTCTGATT TTTCTCTTTT TTAAAAACAA ACAAACAAAC |
| | | TGTTGCAGAT TATGAGTGAA CCCCCAAATA GGGTGTTTTC TGCCTTCAAA |
| | | TAAAAGTCAC CCCTGCATGG TGAA |
| 28 | S100 | AGCCACATTT GCAACCTTGG CCATCTGTCC AGAACCTGCT CCCACCTCAG |
| | | GCCCAGGCCA ACCGTGCACT GCTGCAATGG GCTCTGAGCT GGAGACGGCG |
| | | ATGGAGACCC TCATCAACGT GTTCCACGCC CACTCGGGCA AAGAGGGGGA |
| | | CAAGTACAAG CTGAGCAAGA AGGAGCTGAA AGAGCTGCTG CAGACGGAGC |
| | | TCTCTGGCTT CCTGGATGCC CAGAAGGATG TGGATGCTGT GGACAAGGTG |
| | | ATGAAGGAGC TAGACGAGAA TGGAGACGGG GAGGTGGACT TCCAGGAGTA |
| | | TGTGGTGCTT GTGGCTGCTC TCACAGTGGC CTGTAACAAT TTCTTCTGGG |
| | | AGAACAGTTG AGCAGACAGC CACATTGGGC AGCGCCCTTC CTCTCCACCC |
| | | TCCCAGACCT GCCTCTTCCC CCTGCTTCCA CCTCACCCCA CTTATCCCTC |
| | | TCCATAACCC CACCCTTGCC CACCCCACCC CCACCCCCAC CAAGGGCGCA |
| | | AGAGTAGCGG TCCAAGCCTG CAACTCATCT TTCATTAAAG GCTTCTCTCT |
| | | CACCAGCCA |
| 29 | MBP | AGTCACCGCC GCCGCGCGCC AGAGAGAAGC AGCCTCCGGC CCCGGCGGCC |
| | | CCTGTCTCCC GACCCCGGAA GGCGAAGCAG GCTGCCCGGG GACCCCGCGC |
| | | GTGGGCGCTT GAAGCCGAGA CCAGCCTGCC CGGGCCTGGG CAGGCGGAGC |
| | | AGGGCCTTGG ACCCCGCGGC GCCCCTCGGC CTCGGAGCAA CGAGCGCAGC |
| | | GCCGCCTCTG AAGAGCCAAT CCATTCAGGA TGGGAAACCA CGCAGGCAAA |
| | | CGAGAATTAA ATGCCGAGAA GGCCAGTACG AATAGTGAAA CTAACAGAGG |
| | | AGAATCTGAA AAAAAGAGAA ACCTGGGTGA ACTTTCACGG ACAACCTCAG |
| | | AGGACAACGA AGTGTTCGGA GAGGCAGATG CGAACCAGAA CAATGGGACC |
| | | TCCTCTCAGG ACACAGCGGT GACTGACTCC AAGCGCACAG CGGACCCGAA |
| | | GAATGCCTGG CAGGATGCCC ACCCAGCTGA CCCAGGGAGC CGCCCCCACT |
| | | TGATCCGCCT CTTTTCCCGA GATGCCCCGG GGAGGGAGGA CAACACCTTC |
| | | AAAGACAGGC CCTCTGAGTC CGACGAGCTC CAGACCATCC AAGAAGACAG |
| | | TGCAGCCACC TCCGAGAGCC TGGATGTGAT GGCGTCACAG AAGAGACCCT |
| | | CCCAGAGGCA CGGATCCAAG TACCTGGCCA CAGCAAGTAC CATGGACCAT |
| | | GCCAGGCATG GCTTCCTCCC AAGGCACAGA GACACGGGCA TCCTTGACTC |
| | | CATCGGGCGC TTCTTTGGCG GTGACAGGGG TGCGCCCAAG CGGGGCTCTG |
| | | GCAAGGACTC ACACCACCCG GCAAGAACTG CTCACTACGG CTCCCTGCCC |
| | | CAGAAGTCAC ACGGCCGGAC CCAAGATGAA AACCCCGTAG TCCACTTCTT |
| | | CAAGAACATT GTGACGCCTC GCACACCACC CCCGTCGCAG GGAAAGGGGA |
| | | GAGGACTGTC CCTGAGCAGA TTTAGCTGGG GGGCCGAAGG CCAGAGACCA |
| | | GGATTTGGCT ACGGAGGCAG AGCGTCCGAC TATAAATCGG CTCACAAGGG |
| | | ATTCAAGGGA GTCGATGCCC AGGGCACGCT TTCCAAAATT TTTAAGCTGG |
| | | GAGGAAGAGA TAGTCGCTCT GGATCACCCA TGGCTAGACG CTGAAAACCC |
| | | ACCTGGTTCC GGAATCCTGT CCTCAGCTTC TTAATATAAC TGCCTTAAAA |
| | | CTTTAATCCC ACTTGCCCCT GTTACCTAAT TAGAGCAGAT GACCCCTCCC |
| | | CTAATGCCTG CGGAGTTGTG CACGTAGTAG GGTCAGGCCA CGGCAGCCTA |
| | | CCGGCAATTT CCGGCCAACA GTTAAATGAG AACATGAAAA CAGAAAACGG |
| | | TTAAAACTGT CCCTTTCTGT GTGAAGATCA CGTTCCTTCC CCCGCAATGT |
| | | GCCCCCAGAC GCACGTGGGT CTTCAGGGGG CCAGGTGCAC AGACGTCCCT |
| | | CCACGTTCAC CCCTCCACCC TTGGACTTTC TTTTCGCCGT GGCTGCGGCA |
| | | CCCTTGCGCT TTTGCTGGTC ACTGCCATGG AGGCACACAG CTGCAGAGAC |
| | | AGAGAGGACG TGGGCGGCAG AGAGGACTGT TGACATCCAA GCTTCCTTTG |
| | | TTTTTTTTTC CTGTCCTTCT CTCACCTCCT AAAGTAGACT TCATTTTTCC |
| | | TAACAGGATT AGACAGTCAA GGAGTGGCTT ACTACATGTG GGAGCTTTTG |
| | | GTATGTGACA TGCGGGCTGG GCAGCTGTTA GAGTCCAACG TGGGGCAGCA |
| | | CAGAGAGGGG GCCACCTCCC CAGGCCGTGG CTGCCCACAC ACCCCAATTA |
| | | GCTGAATTCG CGTGTGGCAG AGGGAGGAAA AGGAGGCAAA CGTGGGCTGG |
| | | GCAATGGCCT CACATAGGAA ACAGGGTCTT CCTGGAGATT TGGTGATGGA |
| | | GATGTCAAGC AGGTGGCCTC TGGACGTCAC CGTTGCCCTG CATGGTGGCC |
| | | CCAGAGCAGC CTCTATGAAC AACCTCGTTT CCAAACCACA GCCCACAGCC |
| | | GGAGAGTCCA GGAAGACTTG CGCACTCAGA GCAGAAGGGT AGGAGTCCTC |
| | | TAGACAGCCT CGCAGCCGCG CCAGTCGCCC ATAGACACTG GCTGTGACCG |
| | | GGCGTGCTGG CAGCGGCAGT GCACAGTGGC CAGCACTAAC CCTCCCTGAG |
| | | AAGATAACCG GCTCATTCAC TTCCTCCCAG AAGACGCGTG GTAGCGAGTA |
| | | GGCACAGGCG TGCACCTGCT CCCGAATTAC TCACCGAGAC ACACGGGCTG |
| | | AGCAGACGGC CCCGTGGATG GAGACAAAGA GCTCTTCTGA CCATATCCTT |
| | | CTTAACACCC GCTGGCATCT CCTTTCGCGC CTCCCTCCCT AACCTACTGA |
| | | CCCACCTTTT GATTTTAGCG CACCTGTGAT TGATAGGCCT TCCAAAGAGT |
| | | CCCACGCTGG CATCACCCTC CCCGAGGACG GAGATGAGGA GTAGTCAGCG |
| | | TGATGCCAAA ACGCGTCTTC TTAATCCAAT TCTAATTCTG AATGTTTCGT |
| | | GTGGGCTTAA TACCATGTCT ATTAATATAT AGCCTCGATG ATGAGAGAGT |
| | | TACAAAGAAC AAAACTCCAG ACACAAACCT CCAAATTTTT CAGCAGAAGC |
| | | ACTCTGCGTC GCTGAGCTGA GGTCGGCTCT GCGATCCATA CGTGGCCGCA |
| | | CCCACACAGC ACGTGCTGTG ACGATGGCTG AACGGAAAGT GTACACTGTT |
| | | CCTGAATATT GAAATAAAAC AATAAACTTT TAATGGTA |
| 30 | GFAP | AGAGCCAGAG CAGGATGGAG AGGAGACGCA TCACCTCCGC TGCTCGCCGC |
| | | TCCTACGTCT CCTCAGGGGA GATGATGGTG GGGGGCCTGG CTCCTGGCCG |
| | | CCGTCTGGGT CCTGGCACCC GCCTCTCCCT GGCTCGAATG CCCCCTCCAC |
| | | TCCCGACCCG GGTGGATTTC TCCCTGGCTG GGGCACTCAA TGCTGGCTTC |
| | | AAGGAGACCC GGGCCAGTGA GCGGGCAGAG ATGATGGAGC TCAATGACCG |
| | | CTTTGCCAGC TACATCGAGA AGGTTCGCTT CCTGGAACAG CAAAACAAGG |
| | | CGCTGGCTGC TGAGCTGAAC CAGCTGCGGG CCAAGGAGCC CACCAAGCTG |
| | | GCAGACGTCT ACCAGGCTGA GCTGCGAGAG CTGCGGCTGC GGCTCGATCA |
| | | ACTCACCGCC AACAGCGCCC GGCTGGAGGT TGAGAGGGAC AATCTGGCAC |
| | | AGGACCTGGC CACTGTGAGG CAGAAGCTCC AGGATGAAAC CAGATGAAGC |
| | | CACCCTGGCC CGTCTGGATC TGGAGAGGAA GATTGAGTCG CTGGAGGAGG |
| | | AGATCCGGTT CTTGAGGAAG ATCCACGAGG AGGAGGTTCG GGAACTCCAG |
| | | GAGCAGCTGG CCCGACAGCA GGTCCATGTG GAGCTTGACG TGGCCAAGCC |
| | | AGACCTCACC GCAGCCCTGA AAGAGATCCG CACGCAGTAT GAGGCAATGG |
| | | CGTCCAGCAA CATGCATGAA GCCGAAGAGT GGTACCGCTC CAAGTTTGCA |
| | | GACCTGACAG ACGCTGCTGC CCGCAACGCG GAGCTGCTCC GCCAGGCCAA |
| | | GCACGAAGCC AACGACTACC GGCGCCAGTT GCAGTCCTTG ACCTGCGACC |
| | | TGGAGTCTCT GCGCGGCACG AACGAGTCCC TGGAGAGGCA GATGCGCGAG |
| | | CAGGAGGAGC GGCACGTGCG GGAGGCGGCC AGTTATCAGG AGGCGCTGGC |
| | | GCGGCTGGAG GAAGAGGGGC AGAGCCTCAA GGACGAGATG GCCCGCCACT |
| | | TGCAGGAGTA CCAGGACCTG CTCAATGTCA AGCTGGCCCT GGACATCGAG |
| | | ATCGCCACCT ACAGGAAGCT GCTAGAGGGC GAGGAGAACC GGATCACCAT |
| | | TCCCGTGCAG ACCTTCTCCA ACCTGCAGAT TCGAGAAACC AGCCTGGACA |
| | | CCAAGTCTGT GTCAGAAGGC CACCTCAAGA GGAACATCGT GGTGAAGACC |
| | | GTGGAGATGC GGGATGGAGA GGTCATTAAG GAGTCCAAGC AGGAGCACAA |
| | | GGATGTGATG TGAGGCAGGA CCCACCTGGT GGCCTCTGCC CCGTCTCATG |
| | | AGGGGCCCGA GCAGAAGCAG GATAGTTGCT CCGCCTCTGC TGGCACATTT |
| | | CCCCAGACCT GAGCTCCCCA CCACCCCAGC TGCTCCCCTC CCTCCTCTGT |
| | | CCCTAGGTCA GCTTGCTGCC CTAGGCTCCG TCAGTATCAG GCCTGCCAGA |
| | | CGGCACCCAC CCAGCACCCA GCAACTCCAA CTAACAAGAA ACTCACCCCC |
| | | AAGGGGCAGT CTGGAGGGGC ATGGCCAGCA GCTTGCGTTA GAATGAGGAG |
| | | GAAGGAGAGA AGGGGAGGAG GGCGGGGGGC ACCTACTACA TCGCCCTCCA |
| | | CATCCCTGAT TCCTGTTGTT ATGGAAACTG TTGCCAGAGA TGGAGGTTCT |
| | | CTCGGAGTAT CTGGGAACTG TGCCTTTGAG TTTCCTCAGG CTGCTGGAGG |
| | | AAAACTGAGA CTCAGACAGG AAAGGGAAGG CCCCACAGAC AAGGTAGCCC |
| | | TGGCCAGAGG CTTGTTTTGT CTTTTGGTTT TTATGAGGTG GGATATCCCT |
| | | ATGCTGCCTA GGCTGACCTT GAACTCCTGG GCTCAAGCAG TCTACCCACC |
| | | TCAGCCTCCT GTGTAGCTGG GATTATAGAT TGGAGCCACC ATGCCCAGCT |
| | | CAGAGGGTTG TTCTCCTAGA CTGACCCTGA TCAGTCTAAG ATGGGTGGGG |
| | | ACGTCCTGCC ACCTGGGGCA GTCACCTGCC CAGATCCCAG AAGGACCTCC |
| | | TGAGCGATGA CTCAAGTGTC TCAGTCCACC TGAGCTGCCA TCCAGGGATG |
| | | CCATCTGTGG GCACGCTGTG GGCAGGTGGG AGCTTGATTC TCAGCACTTG |
| | | GGGGATCTGT TGTGTACGTG GAGAGGGATG AGGTGCTGGG AGGGATAGAG |
| | | GGGGGCTGCC TGGCCCCCAG CTGTGGGTAC AGAGAGGTCA AGCCCAGGAG |
| | | GACTGCCCCG TGCAGACTGG AGGGGACGCT GGTAGAGATG GAGGAGGAGG |
| | | CAATTGGGAT GGCGCTAGGC ATACAAGTAG GGGTTGTGGG TGACCAGTTG |
| | | CACTTGGCCT CTGGATTGTG GGAATTAAGG AAGTGACTCA TCCTCTTGAA |
| | | GATGCTGAAA CAGGAGAGAA AGGGGATGTA TCCATGGGGG CAGGGCATGA |
| | | CTTTGTCCCA TTTCTAAAGG CCTCTTCCTT GCTGTGTCAT ACCAGGCCGC |
| | | CCCAGCCTCT GAGCCCCTGG GACTGCTGCT TCTTAACCCC AGTAAGCCAC |
| | | TGCCACACGT CTGACCCTCT CCACCCCATA GTGACCGGCT GCTTTTCCCT |
| | | AAGCCAAGGG CCTCTTGCGG TCCCTTCTTA CTCACACACA AAATGTACCC |
| | | AGTATTCTAG GTAGTGCCCT ATTTTACAAT TGTAAAACTG AGGCACGAGC |
| | | AAAGTGAAGA CACTGGCTCA TATTCCTGCA GCCTGGAGGC CGGGTGCTCA |
| | | GGGCTGACAC GTCCACCCCA GTGCACCCAC TCTGCTTTGA CTGAGCAGAC |
| | | TGGTGAGCAG ACTGGTGGGA TCTGTGCCCA GAGATGGGAC TGGGAGGGCC |
| | | CACTTCAGGG TTCTCCTCTC CCCTCTAAGG CCGAAGAAGG GTCCTTCCCT |
| | | CTCCCCAAGA CTTGGTGTCC TTTCCCTCCA CTCCTTCCTG CCACCTGCTG |
| | | CTGCTGCTGC TGCTAATCTT CAGGGCACTG CTGCTGCCTT TAGTCGCTGA |
| | | GGAAAAATAA AGACAAATGC TGCGCCCTTC CCCAGAGTGG ACTCTGATCT |
| | | GTTCATGAGA GGGCGGGACT GGGGCCAAGA TGTAGCCTTT GACAAGACCA |
| | | ACTCATTTCT TATTACTGAT CATCTCTGGG GCCCATGCCC TCACCAAATT |
| | | CCACCCGCAG CCAAAGAGGA CATACACCAG CTCCCTCCAC TCTTTTCTTC |
| | | CTTCCTCTCC CTGCTACCTG CAACTCAACC AGCACAATCT TCATAGGCAA |
| | | GAAAGCAAAG CAGCTCAAAC ATGATTCAAC ACTGATCAGT GTTTACCACT |
| | | GGATAAATCT GAGTTCACAC TTTCCTTCTC TGACCTAAAT GTGAAGTCAG |
| | | GAAACACATG TGCCCTACTT CCATCCTGAG CTCAGTCCCC AATCTCCCAC |
| | | CAGCCTCAGG CCCCTCCACT TCTCAGATCA GGTCCCAGAC CTGCCCATGA |
| | | AAATGGGGAG CAGGCTGTAA CAGATTTGTC CACATGTTCC TACCACCTGT |
| | | CCCAACCCAG GGTACCCACC CAGAGACATC TGGTATCATT TAACAAACAC |
| | | ATTGAAGGAC AACTGGTCTT CAGAGCTGAA GAGAGCTCCT AGGGGGAGAA |
| | | GCTGGGACAA CAGTGAAATA AGTAGCAGCA GCAACGACAG AAGTGAATGG |
| | | TGACAAAGAC TGCTGTGATG AGCAGGTAGC CTATCAGGGT GAGCTCCACA |
| | | GCCGAGCGAG TCTCAGGATC TGAGAACGAG GCTGGGTAGT GCCCATGAGA |
| | | TGTCACACCC AGCCGGAAGC CAGCAACTAG CACACCCTGC CTCCAGCAAT |
| | | AGTAGATGCC CCGGTCATCC AGCTGGGTGA AGCGGATGTG GAGCTGGTTG |
| | | CCGTGGTCAA TGAACACCCT CATGGACCTG TTGACACCCT TCAGGTACTG |
| | | TGTGCGGTAG AGGTGCTGGC GGTCTTTGTC CCAGGCCACT GCATGCTCTG |
| | | GCCGGGCCCC AGGACAGGAG ATGATGAGTC CATGGCCCAG TCTCTGCTGG |
| | | TGGAACTGAA TGGGCACCTG GGGCACCCAG GGCCGGCTGC CCACTTTGGA |
| | | CACATAGTTA ATGATGGCCA GCACGCCCTC CCGGATGGTC TTTGTCTTCT |
| | | CACAGGGTAC TAAGCAGCTC CGAACCAGCA CCTCAGGCGT GTGGTCCCTG |
| | | GCCTTGGTCC GCAGCTTCCT TGGCACAGCC CTTGAGCCAC AAGACACCAC |
| | | ATCGGGCACG GCCTTGAGGT AGCGTGGGGA GAGGTCTGGG CTCTGCAGGT |
| | | AGCAGAGGCC GATGCGCCAC TGCTCCCCAC GCACTCCGCA GCGGTCACAG |
| | | GGGGTCCATT CCCAGAAGGT GGTGAAGACA TGGAGGTGCC CATAGTATTC |
| | | ATCTGCAAAG GGCTCCTGGC CCTTGTCCTG GAAAGTGGCC ACCATTCCCT |
| | | CACTGTTCTG GATGTCCACA TCGTAGGCGT AAAAGTAGTC CCCCTTGCGG |
| | | GTGCCGCAGA AGTACAGGCC TGAGTCCTCA GACTGAGCCC TGAAAACCAA |
| | | CAAGCTGAAC ATGCGGATGC TGAAGCGGGT CAGCATGTCG CTGCCCACAC |
| | | GTACCTGGGC TGCCTCCGTC AGCACCCGCC CATCAAAGTC CGTCAGCACT |
| | | TTGGTGTGGC TGCTACCTAG GTGCTTTTGG TAGAACCAGA CTACAGCTGG |
| | | CACCTCTTCG GGTTTGCAGT GACAGGGAAG CTCAAAGCTC ATGTCGGCCA |
| | | GGTAGGCTGC ATTTTCAAAC ATCAGGAAAG CAGGGCAGGG GGTCCTCTGA |
| | | AAAATGTTTT CCTTCTCCAC AATTTCAAAG GCCTGGAGCC CCCATGCCCA |
| | | CAGGAGCACA GTGGTGAGGG CCAGGTGCAT ACCTGAAGGA GGCAGGGGTC |
| | | AGAGGGGCAG GGCAAAACCA GGGCATTAAA GGCTCATAGG GCTCCTAGAA |
| | | AGCTCTGCTA AGCGGAAGCC TCTAGATGAG GAAAGGATTA TGCAGCCAGG |
| | | AAAAGCAGCA ACAATCTGCA GAGGAAGCCG CCAAGTGCAA GGCAATTTAT |
| | | TCCCAGTGGA TGTACAAGAT GCCCTTCTAA CATTCCAGAC CTGATCTCAG |
| | | GGTGGGGGGG GAAAGCCATT CTAGAACCTG GCCTTTACTC CCCTTTCTAG |
| | | AACACTGGCG CTCACCCAAG AATGGGTCAA AGGAAACCGG AATGAGAAGG |
| | | GCGGGCCGAG GTGCTCGGGC AGGGAGATCT CTGCCTCAGT GCTCCAGGCC |
| | | CTGCCCTGCC AGCCTGGTGG AAAAGTCTTT CATCAACCTG GGGGATGAAG |
| | | GAAACCCACC CTCCTGCATA TCTGGCCATC CGGGAGGCTG GCTGGACCTG |
| | | AGCTGATGGC TTGGGACTTT CCCAGGCCCA ACCTGCACAA GAACTGAGTC |
| | | TCTAGGGGAA AATTCAACAC CTCAAATGAT GTAGTATTTG ATCATTTGTT |
| | | GATTACATGT CCATTCATTG GTTTGGGGCT ATAAACATTC TTGTTAAGAG |
| | | CTGTGGAGAT CAGTGTTTGT TTACCATAAA GATTTTGCTT TTTCCCTTTT |
| | | A |
| 31 | PMP22 | AGTTACAGGG AGCACCACCA GGGAACATCT CGGGGAGCCT GGTTGGAAGC |
| | | TGCAGGCTTA GTCTGTCGGC TGCGGGTCTC TGACTGCCCT GTGGGGAGGG |
| | | TCTTGCCTTA ACATCCCTTG CATTTGGCTG CAAAGAAATC TGCTTGGAAG |
| | | AAGGGGTTAC GCTGTTTGGC CGGGCAGAAA CTCCGCTGAG CAGAACTTGC |
| | | CGCCAGAATG CTCCTCCTGT TGCTGAGTAT CATCGTCCTC CACGTCGCGG |
| | | TGCTGGTGCT GCTGTTCGTC TCCACGATCG TCAGCCAATG GATCGTGGGC |
| | | AATGGACACG CAACTGATCT CTGGCAGAAC TGTAGCACCT CTTCCTCAGG |
| | | AAATGTCCAC CACTGTTTCT CATCATCACC AAACGAATGG CTGCAGTCTG |
| | | TCCAGGCCAC CATGATCCTG TCGATCATCT TCAGCATTCT GTCTCTGTTC |
| | | CTGTTCTTCT GCCAACTCTT CACCCTCACC AAGGGGGGCA GGTTTTACAT |
| | | CACTGGAATC TTCCAAATTC TTGCTGGTCT GTGCGTGATG AGTGCTGCGG |
| | | CCATCTACAC GGTGAGGCAC CCGGAGTGGC ATCTCAACTC GGATTACTCC |
| | | TACGGTTTCG CCTACATCCT GGCCTGGGTG GCCTTCCCCC TGGCCCTTCT |
| | | CAGCGGTGTC ATCTATGTGA TCTTGCGGAA ACGCGAATGA GGCGCCCAGA |
| | | CGGTCTGTCT GAGGCTCTGA GCGTACATAG GGAAGGGAGG AAGGGAAAAC |
| | | AGAAAGCAGA CAAAGAAAAA AGAGCTAGCC CAAAATCCCA AACTCAAACC |
| | | AAACCAAACA GAAAGCAGTG GAGGTGGGGG TTGCTGTTGA TTGAAGATGT |
| | | ATATAATATC TCCGGTTTAT AAAACCTATT TATAACACTT TTTACATATA |
| | | TGTACATAGT ATTGTTTGCT TTTTATGTTG ACCATCAGCC TCGTGTTGAG |
| | | CCTTAAAGAA GTAGCTAAGG AACTTTACAT CCTAACAGTA TAATCCAGCT |
| | | CAGTATTTTT GTTTTGTTTT TTGTTTGTTT GTTTTGTTTT ACCCAGAAAT |
| | | AAGATAACTC CATCTCGCCC CTTCCCTTTC ATCTGAAAGA AGATACCTCC |
| | | CTCCCAGTCC ACCTCATTTA GAAAACCAAA GTGTGGGTAG AAACCCCAAA |
| | | TGTCCAAAAG CCCTTTTCTG GTGGGTGACC CAGTGCATCC AACAGAAACA |
| | | GCCGCTGCCC GAACCTCTGT GTGAAGCTTT ACGCGCACAC GGACAAAATG |
| | | CCCAAACTGG AGCCCTTGCA AAAACACGGC TTGTGGCATT GGCATACTTG |
| | | CCCTTACAGG TGGAGTATCT TCGTCACACA TCTAAATGAG AAATCAGTGA |
| | | CAACAAGTCT TTGAAATGGT GCTATGGATT TACCATTCCT TATTATCACT |
| | | AATCATCTAA ACAACTCACT GGAAATCCAA TTAACAATTT TACAACATAA |
| | | GATAGAATGG AGACCTGAAT AATTCTGTGT AATATAAATG GTTTATAACT |
| | | GCTTTTGTAC CTAGCTAGGC TGCTATTATT ACTATAATGA GTAAATCATA |
| | | AAGCCTTCAT CACTCCCACA TTTTTCTTAC GGTCGGAGCA TCAGAACAAG |
| | | CGTCTAGACT CCTTGGGACC GTGAGTTCCT AGAGCTTGGC TGGGTCTAGG |
| | | CTGTTCTGTG CCTCCAAGGA CTGTCTGGCA ATGACTTGTA TTGGCCACCA |
| | | ACTGTAGATG TATATATGGT GCCCTTCTGA TGCTAAGACT CCAGACCTTT |
| | | TGTTTTTGCT TTGCATTTTC TGATTTTATA CCAACTGTGT GGACTAAGAT |
| | | GCATTAAAAT AAACATCAGA GTAACTCA |
| 32 | CD6 | ACTCACAGGT TGGGTTTGAT CGCATGCGTG TCGGAGAGGA GAGAGCAGAG |
| | | AGAGACACAG GAACAAGAAC AGCAAAGGGT AGAGCAGACC TGCGCCAGGG |
| | | GCGCACAACG GCCGTGTCCA CCTCCCGGCC CCAAGATGGT GCTTCCCACA |
| | | GGCAGCCACG CGTAGCAGCC AGAGACAGCT CCAGACATGT GGCTCTTCTT |
| | | CGGGATCACT GGATTGCTGA CGGCAGCCCT CTCAGGTCAT CCATCTCCAG |
| | | CCCCACCTGA CCAGCTCAAC ACCAGCAGTG CAGAGAGTGA GCTCTGGGAG |
| | | CCAGGGGAGC GGCTTCCGGT CCGTCTGACA AACGGGAGCA GCAGCTGCAG |
| | | CGGGACGGTG GAGGTGCGGC TCGAGGCGTC CTGGGAGCCC GCGTGCGGGG |
| | | CGCTCTGGGA CAGCCGCGCC GCCGAGGCCG TGTGCCGAGC ACTGGGCTGC |
| | | GGCGGGGCGG AGGCCGCCTC TCAGCTCGCC CCGCCGACCC CTGAGCTGCC |
| | | GCCCCCGCCT GCAGCCGGGA ACACCAGCGT AGCAGCTAAT GCCACTCTGG |
| | | CCGGGGCGCC CGCCCTCCTG TGCAGCGGCG CCGAGTGGCG GCTCTGCGAG |
| | | GTGGTGGAGC ACGCGTGCCG CAGCGACGGG AGGCGGGCCC GTGTCACCTG |
| | | TGCAGAGAAC CGCGCGCTGC GCCTGGTGGA CGGTGGCGGC GCCTGCGCCG |
| | | GCCGCGTGGA GATGCTGGAG CATGGCGAGT GGGGATCAGT GTGCGATGAC |
| | | ACTTGGGACC TGGAGGACGC CCACGTGGTG TGCAGGCAAC TGGGCTGCGG |
| | | CTGGGCAGTC CAGGCCCTGC CCGGCTTGCA CTTCACGCCC GGCCGCGGGC |
| | | CTATCCACCG GGACCAGGTG AACTGCTCGG GGGCCGAAGC TTACCTGTGG |
| | | GACTGCCCGG GGCTGCCAGG ACAGCACTAC TGCGGCCACA AAGAGGACGC |
| | | GGGCGCGGTG TGCTCAGAGC ACCAGTCCTG GCGCCTGACA GGGGGCGCTG |
| | | ACCGCTGCGA GGGGCAGGTG GAGGTACACT TCCGAGGGGT CTGGAACACA |
| | | GTGTGTGACA GTGAGTGGTA CCCATCGGAG GCCAAGGTGC TCTGCCAGTC |
| | | CTTGGGCTGT GGAACTGCGG TTGAGAGGCC CAAGGGGCTG CCCCACTCCT |
| | | TGTCCGGCAG GATGTACTAC TCATGCAATG GGGAGGAGCT CACCCTCTCC |
| | | AACTGCTCCT GGCGGTTCAA CAACTCCAAC CTCTGCAGCC AGTCGCTGGC |
| | | AGCCAGGGTC CTCTGCTCAG CTTCCCGGAG TTTGCACAAT CTGTCCACTC |
| | | CCGAAGTCCC TGCAAGTGTT CAGACAGTCA CTATAGAATC TTCTGTGACA |
| | | GTGAAAATAG AGAACAAGGA ATCTCGGGAG CTAATGCTCC TCATCCCCTC |
| | | CATCGTTCTG GGAATTCTCC TCCTTGGCTC CCTCATCTTC ATAGCCTTCA |
| | | TCCTCTTGAG AATTAAAGGA AAATATGCCC TCCCCGTAAT GGTGAACCAC |
| | | CAGCACCTAC CCACCACCAT CCCGGCAGGG AGCAATAGCT ATCAACCGGT |
| | | CCCCATCACC ATCCCCAAAG AAGTTTTCAT GCTGCCCATC CAGGTCCAGG |
| | | CCCCGCCCCC TGAGGACTCA GACTCTGGCT CGGACTCAGA CTATGAGCAC |
| | | TATGACTTCA GCGCCCAGCC TCCTGTGGCC CTGACCACCT TCTACAATTC |
| | | CCAGCGGCAT CGGGTCACAG ATGAGGAGGT CCAGCAAAGC AGGTTCCAGA |
| | | TGCCACCCTT GGAGGAAGGA CTTGAAGAGT TGCATGCCTC CCACATCCCA |
| | | ACTGCCAACC CTGGACACTG CATTACAGAC CCGCCATCCC TGGGCCCTCA |
| | | GTATCACCCG AGGAGCAACA GTGAGTCGAG CACCTCTTCA GGGGAGGATT |
| | | ACTGCAATAG TCCCAAAAGC AAGCTGCCTC CATGGAACCC CCAGGTGTTT |
| | | TCTTCAGAGA GGAGTTCCTT CCTGGAGCAG CCCCCAAACT TGGAGCTGGC |
| | | CGGCACCCAG CCAGCCTTTT CAGCAGGGCC CCCGGCTGAT GACAGCTCCA |
| | | GCACCTCATC CGGGGAGTGG TACCAGAACT TCCAGCCACC ACCCCAGCCC |
| | | CCTTCGGAGG AGCAGTTTGG CTGTCCAGGG TCCCCCAGCC CTCAGCCTGA |
| | | CTCCACCGAC AACGATGACT ACGATGACAT CAGCGCAGCC TAGGCCGGGG |
| | | CCAGCCGAGG CCCCTTTCCC ACCCTCCCAG CTCACCTCCC CATGGAGCTG |
| | | AGAGGCCTCC CTTGGAGAGA TGGAAGGAAA CGTTATACCT TGTACCCCTC |
| | | GGTCTCCATC CATCAAGCCA AACCTGCTGC CACAGCCCTC CCCCGGCCCC |
| | | AGATAGCAGC CCCAGGGAGG ATGCTGCCTC CAAGAGGTGT GAGCCCTCTG |
| | | TCTCGGGGAT GAACAAGCAG AGTCTGGGCT ACCTCTTGAC AGCTGGTGGA |
| | | GGGGAGTTGG GGAGCTGGAC TGGATGACTC TGGAGGCCCC TTCCAAACCT |
| | | CAAGTGTCCG GCGCTTTGAT TGCCTGAGTT TCTGACACTT CAGGGCCCAG |
| | | AGGTCCTGCG AGGGGCAGAA CTGGACCCCC ATGCCAGTGC TGCTGCAGGA |
| | | GGGCCCATAT ACTAGGGTCT GCTGAGCTGT TGTCACTGAT CGGTGGGCGC |
| | | TGGGGGGGTA GGGTAGCACA CCAGCTGTCC CAGGCTTTGC TCCGGGCGGT |
| | | AACTGCACTT GGGCAGGGAA TATAGCCTTC CTGGGCACAA CTAGCTGACA |
| | | ATGACAGGTT GACTGTGTAC CCCCAACCAA GGAGCTGGGG CCCAAGGCCA |
| | | GTCCTGCCCC AGAGACACTC CAAGTCCGCC AGGGGCACAG ACCAGTTCTG |
| | | CAGTGACTGT CCCTGGACAA TGGGTCTTTA TTCTGAGTTT CCTATGGTTT |
| | | ACAAAGAGGG CCCCAGCCCA GCCCCACCAC AGATCCCAGA GATAGGGGCC |
| | | CAGTCTCCAT GGGGGCAAGG AGCATAGAGA TGTTTTCCAG GAAGGGGCTC |
| | | AGAAGCTGCA CTAGGCCCCG AGTCCCCATG TGTCTCCTTG AATTGATGAG |
| | | GATGCTCCTG GGAGGGATGC GTGACTATGT GGTGTTGCAC CCGGGGCTGC |
| | | AAACGTCTCC GTGCAGCCCC CAGAGAGAGG CCCATGGGCT CAGACCAGGC |
| | | TTTGTTGTCC TGCTCTGAGT ATCCTGAGAT TAAACTGAAT TGCTGAATGA |
| | | AA |
| 33 | CD9 | AGCCGCCTGC ATCTGTATCC AGCGCCAGGT CCCGCCAGTC CCAGCTGCGC |
| | | GCGCCCCCCA GTCCCGCACC CGTTCGGCCC AGGCTAAGTT AGCCCTCACC |
| | | ATGCCGGTCA AAGGAGGCAC CAAGTGCATC AAATACCTGC TGTTCGGATT |
| | | TAACTTCATC TTCTGGCTTG CCGGGATTGC TGTCCTTGCC ATTGGACTAT |
| | | GGCTCCGATT CGACTCTCAG ACCAAGAGCA TCTTCGAGCA AGAAACTAAT |
| | | AATAATAATT CCAGCTTCTA CACAGGAGTC TATATTCTGA TCGGAGCCGG |
| | | CGCCCTCATG ATGCTGGTGG GCTTCCTGGG CTGCTGCGGG GCTGTGCAGG |
| | | AGTCCCAGTG CATGCTGGGA CTGTTCTTCG GCTTCCTCTT GGTGATATTC |
| | | GCCATTGAAA TAGCTGCGGC CATCTGGGGA TATTCCCACA AGGATGAGGT |
| | | GATTAAGGAA GTCCAGGAGT TTTACAAGGA CACCTACAAC AAGCTGAAAA |
| | | CCAAGGATGA GCCCCAGCGG GAAACGCTGA AAGCCATCCA CTATGCGTTG |
| | | AACTGCTGTG GTTTGGCTGG GGGCGTGGAA CAGTTTATCT CAGACATCTG |
| | | CCCCAAGAAG GACGTACTCG AAACCTTCAC CGTGAAGTCC TGTCCTGATG |
| | | CCATCAAAGA GGTCTTCGAC AATAAATTCC ACATCATCGG CGCAGTGGGC |
| | | ATCGGCATTG CCGTGGTCAT GATATTTGGC ATGATCTTCA GTATGATCTT |
| | | GTGCTGTGCT ATCCGCAGGA ACCGCGAGAT GGTCTAGAGT CAGCTTACAT |
| | | CCCTGAGCAG GAAAGTTTAC CCATGAAGAT TGGTGGGATT TTTTGTTTGT |
| | | TTGTTTTGTT TTGTTTGTTG TTTGTTGTTT GTTTTTTTGC CACTAATTTT |
| | | AGTATTCATT CTGCATTGCT AGATAAAAGC TGAAGTTACT TTATGTTTGT |
| | | CTTTTAATGC TTCATTCAAT ATTGACATTT GTAGTTGAGC GGGGGGTTTG |
| | | GTTTGCTTTG GTTTATATTT TTTCAGTTGT TTGTTTTTGC TTGTTATATT |
| | | AAGCAGAAAT CCTGCAATGA AAGGTACTAT ATTTGCTAGA CTCTAGACAA |
| | | GATATTGTAC ATAAAAGAAT TTTTTTGTCT TTAAATAGAT ACAAATGTCT |
| | | ATCAACTTTA ATCAAGTTGT AACTTATATT GAAGACAATT TGATACATAA |
| | | TAAAAAATTA TGACAATGTC CTGGA |
| 34 | CD44 | CTCATTGCCC AGCGGACCCC AGCCTCTGCC AGGTTCGGTC CGCCATCCTC |
| | | GTCCCGTCCT CCGCCGGCCC CTGCCCCGCG CCCAGGGATC CTCCAGCTCC |
| | | TTTCGCCCGC GCCCTCCGTT CGCTCCGGAC ACCATGGACA AGTTTTGGTG |
| | | GCACGCAGCC TGGGGACTCT GCCTCGTGCC GCTGAGCCTG GCGCAGATCG |
| | | ATTTGAATAT AACCTGCCGC TTTGCAGGTG TATTCCACGT GGAGAAAAAT |
| | | GGTCGCTACA GCATCTCTCG GACGGAGGCC GCTGACCTCT GCAAGGCTTT |
| | | CAATAGCACC TTGCCCACAA TGGCCCAGAT GGAGAAAGCT CTGAGCATCG |
| | | GATTTGAGAC CTGCAGGTAT GGGTTCATAG AAGGGCACGT GGTGATTCCC |
| | | CGGATCCACC CCAACTCCAT CTGTGCAGCA AACAACACAG GGGTGTACAT |
| | | CCTCACATCC AACACCTCCC AGTATGACAC ATATTGCTTC AATGCTTCAG |
| | | CTCCACCTGA AGAAGATTGT ACATCAGTCA CAGACCTGCC CAATGCCTTT |
| | | GATGGACCAA TTACCATAAC TATTGTTAAC CGTGATGGCA CCCGCTATGT |
| | | CCAGAAAGGA GAATACAGAA CGAATCCTGA AGACATCTAC CCCAGCAACC |
| | | CTACTGATGA TGACGTGAGC AGCGGCTCCT CCAGTGAAAG GAGCAGCACT |
| | | TCAGGAGGTT ACATCTTTTA CACCTTTTCT ACTGTACACC CCATCCCAGA |
| | | CGAAGACAGT CCCTGGATCA CCGACAGCAC AGACAGAATC CCTGCTACCA |
| | | CTTTGATGAG CACTAGTGCT ACAGCAACTG AGACAGCAAC CAAGAGGCAA |
| | | GAAACCTGGG ATTGGTTTTC ATGGTTGTTT CTACCATCAG AGTCAAAGAA |
| | | TCATCTTCAC ACAACAACAC AAATGGCTGG TACGTCTTCA AATACCATCT |
| | | CAGCAGGCTG GGAGCCAAAT GAAGAAAATG AAGATGAAAG AGACAGACAC |
| | | CTCAGTTTTT CTGGATCAGG CATTGATGAT GATGAAGATT TTATCTCCAG |
| | | CACCATTTCA ACCACACCAC GGGCTTTTGA CCACACAAAA CAGAACCAGG |
| | | ACTGGACCCA GTGGAACCCA AGCCATTCAA ATCCGGAAGT GCTACTTCAG |
| | | ACAACCACAA GGATGACTGA TGTAGACAGA AATGGCACCA CTGCTTATGA |
| | | AGGAAACTGG AACCCAGAAG CACACCCTCC CCTCATTCAC CATGAGCATC |
| | | ATGAGGAAGA AGAGACCCCA CATTCTACAA GCACAATCCA GGCAACTCCT |
| | | AGTAGTACAA CGGAAGAAAC AGCTACCCAG AAGGAACAGT GGTTTGGCAA |
| | | CAGATGGCAT GAGGGATATC GCCAAACACC CAAAGAAGAC TCCCATTCGA |
| | | CAACAGGGAC AGCTGCAGCC TCAGCTCATA CCAGCCATCC AATGCAAGGA |
| | | AGGACAACAC CAAGCCCAGA GGACAGTTCC TGGACTGATT TCTTCAACCC |
| | | AATCTCACAC CCCATGGGAC GAGGTCATCA AGCAGGAAGA AGGATGGATA |
| | | TGGACTCCAG TCATAGTATA ACGCTTCAGC CTACTGCAAA TCCAAACACA |
| | | GGTTTGGTGG AAGATTTGGA CAGGACAGGA CCTCTTTCAA TGACAACGCA |
| | | GCAGAGTAAT TCTCAGAGCT TCTCTACATC ACATGAAGGC TTGGAAGAAG |
| | | ATAAAGACCA TCCAACAACT TCTACTCTGA CATCAAGCAA TAGGAATGAT |
| | | GTCACAGGTG GAAGAAGAGA CCCAAATCAT TCTGAAGGCT CAACTACTTT |
| | | ACTGGAAGGT TATACCTCTC ATTACCCACA CACGAAGGAA AGCAGGACCT |
| | | TCATCCCAGT GACCTCAGCT AAGACTGGGT CCTTTGGAGT TACTGCAGTT |
| | | ACTGTTGGAG ATTCCAACTC TAATGTCAAT CGTTCCTTAT CAGGAGACCA |
| | | AGACACATTC CACCCCAGTG GGGGGTCCCA TACCACTCAT GGATCTGAAT |
| | | CAGATGGACA CTCACATGGG AGTCAAGAAG GTGGAGCAAA CACAACCTCT |
| | | GGTCCTATAA GGACACCCCA AATTCCAGAA TGGCTGATCA TCTTGGCATC |
| | | CCTCTTGGCC TTGGCTTTGA TTCTTGCAGT TTGCATTGCA GTCAACAGTC |
| | | GAAGAAGGTG TGGGCAGAAG AAAAAGCTAG TGATCAACAG TGGCAATGGA |
| | | GCTGTGGAGG ACAGAAAGCC AAGTGGACTC AACGGAGAGG CCAGCAAGTC |
| | | TCAGGAAATG GTGCATTTGG TGAACAAGGA GTCGTCAGAA ACTCCAGACC |
| | | AGTTTATGAC AGCTGATGAG ACAAGGAACC TGCAGAATGT GGACATGAAG |
| | | ATTGGGGTGT AACACCTACA CCATTATCTT GGAAAGAAAC AACCGTTGGA |
| | | AACATAACCA TTACAGGGAG CTGGGACACT TAACAGATGC AATGTGCTAC |
| | | TGATTGTTTC ATTGCGAATC TTTTTTAGCA TAAAATTTTC TACTCTTTTT |
| | | GTTTTTTGTG TTTTGTTCTT TAAAGTCAGG TCCAATTTGT AAAAACAGCA |
| | | TTGCTTTCTG AAATTAGGGC CCAATTAATA ATCAGCAAGA ATTTGATCGT |
| | | TCCAGTTCCC ACTTGGAGGC CTTTCATCCC TCGGGTGTGC TATGGATGGC |
| | | TTCTAACAAA AACTACACAT ATGTATTCCT GATCGCCAAC CTTTCCCCCA |
| | | CCAGCTAAGG ACATTTCCCA GGGTTAATAG GGCCTGGTCC CTGGGAGGAA |
| | | ATTTGAATGG GTCCATTTTG CCCTTCCATA GCCTAATCCC TGGGCATTGC |
| | | TTTCCACTGA GGTTGGGGGT TGGGGTGTAC TAGTTACACA TCTTCAACAG |
| | | ACCCCCTCTA GAAATTTTTC AGATGCTTCT GGGAGACACC CAAAGGGTGA |
| | | AGCTATTTAT CTGTAGTAAA CTATTTATCT GTGTTTTTGA AATATTAAAC |
| | | CCTGGATCAG TCCTTTGATC AGTATAATTT TTTAAAGTTA CTTTGTCAGA |
| | | GGCACAAAAG GGTTTAAACT GATTCATAAT AAATATCTGT ACTTCTTCGA |
| | | TCTTCACCTT TTGTGCTGTG ATTCTTCAGT TTCTAAACCA GCACTGTCTG |
| | | GGTCCCTACA ATGTATCAGG AAGAGCTGAG AATGGTAAGG AGACTCTTCT |
| | | AAGTCTTCAT CTCAGAGACC CTGAGTTCCC ACTCAGACCC ACTCAGCCAA |
| | | ATCTCATGGA AGACCAAGGA GGGCAGCACT GTTTTTGTTT TTTGTTTTTT |
| | | GTTTTTTTTT TTTGACACTG TCCAAAGGTT TTCCATCCTG TCCTGGAATC |
| | | AGAGTTGGAA GCTGAGGAGC TTCAGCCTCT TTTATGGTTT AATGGCCACC |
| | | TGTTCTCTCC TGTGAAAGGC TTTGCAAAGT CACATTAAGT TTGCATGACC |
| | | TGTTATCCCT GGGGCCCTAT TTCATAGAGG CTGGCCCTAT TAGTGATTTC |
| | | CAAAAACAAT ATGGAAGTGC CTTTTGATGT CTTACAATAA GAGAAGAAGC |
| | | CAATGGAAAT GAAAGAGATT GGCAAAGGGG AAGGATGATG CCATGTAGAT |
| | | CCTGTTTGAC ATTTTTATGG CTGTATTTGT AAACTTAAAC ACACCAGTGT |
| | | CTGTTCTTGA TGCAGTTGCT ATTTAGGATG AGTTAAGTGC CTGGGGAGTC |
| | | CCTCAAAAGG TTAAAGGGAT TCCCATCATT GGAATCTTAT CACCAGATAG |
| | | GCAAGTTTAT GACCAAACAA GAGAGTACTG GCTTTATCCT CTAACCTCAT |
| | | ATTTTCTCCC ACTTGGCAAG TCCTTTGTGG CATTTATTCA TCAGTCAGGG |
| | | TGTCCGATTG GTCCTAGAAC TTCCAAAGGC TGCTTGTCAT AGAAGCCATT |
| | | GCATCTATAA AGCAACGGCT CCTGTTAAAT GGTATCTCCT TTCTGAGGCT |
| | | CCTACTAAAA GTCATTTGTT ACCTAAACTT ATGTGCTTAA CAGGCAATGC |
| | | TTCTCAGACC ACAAAGCAGA AAGAAGAAGA AAAGCTCCTG ACTAAATCAG |
| | | GGCTGGGCTT AGACAGAGTT GATCTGTAGA ATATCTTTAA AGGAGAGATG |
| | | TCAACTTTCT GCACTATTCC CAGCCTCTGC TCCTCCCTGT CTACCCTCTC |
| | | CCCTCCCTCT CTCCCTCCAC TTCACCCCAC AATCTTGAAA AACTTCCTTT |
| | | CTCTTCTGTG AACATCATTG GCCAGATCCA TTTTCAGTGG TCTGGATTTC |
| | | TTTTTATTTT CTTTTCAACT TGAAAGAAAC TGGACATTAG GCCACTATGT |
| | | GTTGTTACTG CCACTAGTGT TCAAGTGCCT CTTGTTTTCC CAGAGATTTC |
| | | CTGGGTCTGC CAGAGGCCCA GACAGGCTCA CTCAAGCTCT TTAACTGAAA |
| | | AGCAACAAGC CACTCCAGGA CAAGGTTCAA AATGGTTACA ACAGCCTCTA |
| | | CCTGTCGCCC CAGGGAGAAA GGGGTAGTGA TACAAGTCTC ATAGCCAGAG |
| | | ATGGTTTTCC ACTCCTTCTA GATATTCCCA AAAAGAGGCT GAGACAGGAG |
| | | GTTATTTTCA ATTTTATTTT GGAATTAAAT ACTTTTTTCC CTTTATTACT |
| | | GTTGTAGTCC CTCACTTGGA TATACCTCTG TTTTCACGAT AGAAATAAGG |
| | | GAGGTCTAGA GCTTCTATTC CTTGGCCATT GTCAACGGAG AGCTGGCCAA |
| | | GTCTTCACAA ACCCTTGCAA CATTGCCTGA AGTTTATGGA ATAAGATGTA |
| | | TTCTCACTCC CTTGATCTCA AGGGCGTAAC TCTGGAAGCA CAGCTTGACT |
| | | ACACGTCATT TTTACCAATG ATTTTCAGGT GACCTGGGCT AAGTCATTTA |
| | | AACTGGGTCT TTATAAAAGT AAAAGGCCAA CATTTAATTA TTTTGCAAAG |
| | | CAACCTAAGA GCTAAAGATG TAATTTTTCT TGCAATTGTA AATCTTTTGT |
| | | GTCTCCTGAA GACTTCCCTT AAAATTAGCT CTGAGTGAAA AATCAAAAGA |
| | | GACAAAAGAC ATCTTCGAAT CCATATTTCA AGCCTGGTAG AATTGGCTTT |
| | | TCTAGCAGAA CCTTTCCAAA AGTTTTATAT TGAGATTCAT AACAACACCA |
| | | AGAATTGATT TTGTAGCCAA CATTCATTCA ATACTGTTAT ATCAGAGGAG |
| | | TAGGAGAGAG GAAACATTTG ACTTATCTGG AAAAGCAAAA TGTACTTAAG |
| | | AATAAGAATA ACATGGTCCA TTCACCTTTA TGTTATAGAT ATGTCTTTGT |
| | | GTAAATCATT TGTTTTGAGT TTTCAAAGAA TAGCCCATTG TTCATTCTTG |
| | | TGCTGTACAA TGACCACTGT TATTGTTACT TTGACTTTTC AGAGCACACC |
| | | CTTCCTCTGG TTTTTGTATA TTTATTGATG GATCAATAAT AATGAGGAAA |
| | | GCATGATATG TATATTGCTG AGTTGAAAGC ACTTATTGGA AAATATTAAA |
| | | AGGCTAACAT TAAAAGACTA AAGGAAACAG A |
| 35 | CD46 | CACTTCCGCC CCGGGCGCGG CTCGGGCCAC GCCCACCTGT CCTGCAGCAC |
| | | TGGATGCTTT GTGAGTTGGG GATTGTTGCG TCCCATATCT GGACCCAGAA |
| | | GGGACTTCCC TGCTCGGCTG GCTCTCGGTT TCTCTGCTTT CCTCCGGAGA |
| | | AATAACAGCG TCTTCCGCGC CGCGCATGGA GCCTCCCGGC CGCCGCGAGT |
| | | GTCCCTTTCC TTCCTGGCGC TTTCCTGGGT TGCTTCTGGC GGCCATGGTG |
| | | TTGCTGCTGT ACTCCTTCTC CGATGCCTGT GAGGAGCCAC CAACATTTGA |
| | | AGCTATGGAG CTCATTGGTA AACCAAAACC CTACTATGAG ATTGGTGAAC |
| | | GAGTAGATTA TAAGTGTAAA AAAGGATACT TCTATATACC TCCTCTTGCC |
| | | ACCCATACTA TTTGTGATCG GAATCATACA TGGCTACCTG TCTCAGATGA |
| | | CGCCTGTTAT AGAGAAACAT GTCCATATAT ACGGGATCCT TTAAATGGCC |
| | | AAGCAGTCCC TGCAAATGGG ACTTACGAGT TTGGTTATCA GATGCACTTT |
| | | ATTTGTAATG AGGGTTATTA CTTAATTGGT GAAGAAATTC TATATTGTGA |
| | | ACTTAAAGGA TCAGTAGCAA TTTGGAGCGG TAAGCCCCCA ATATGTGAAA |
| | | AGGTTTTGTG TACACCACCT CCAAAAATAA AAAATGGAAA ACACACCTTT |
| | | AGTGAAGTAG AAGTATTTGA GTATCTTGAT GCAGTAACTT ATAGTTGTGA |
| | | TCCTGCACCT GGACCAGATC CATTTTCACT TATTGGAGAG AGCACGATTT |
| | | ATTGTGGTGA CAATTCAGTG TGGAGTCGTG CTGCTCCAGA GTGTAAAGTG |
| | | GTCAAATGTC GATTTCCAGT AGTCGAAAAT GGAAAACAGA TATCAGGATT |
| | | TGGAAAAAAA TTTTACTACA AAGCAACAGT TATGTTTGAA TGCGATAAGG |
| | | GTTTTTACCT CGATGGCAGC GACACAATTG TCTGTGACAG TAACAGTACT |
| | | TGGGATCCCC CAGTTCCAAA GTGTCTTAAA GTGCTGCCTC CATCTAGTAC |
| | | AAAACCTCCA GCTTTGAGTC ATTCAGTGTC GACTTCTTCC ACTACAAAAT |
| | | CTCCAGCGTC CAGTGCCTCA GGTCCTAGGC CTACTTACAA GCCTCCAGTC |
| | | TCAAATTATC CAGGATATCC TAAACCTGAG GAAGGAATAC TTGACAGTTT |
| | | GGATGTTTGG GTCATTGCTG TGATTGTTAT TGCCATAGTT GTTGGAGTTG |
| | | CAGTAATTTG TGTTGTCCCG TACAGATATC TTCAAAGGAG GAAGAAGAAA |
| | | GGCACATACC TAACTGATGA GACCCACAGA GAAGTAAAAT TTACTTCTCT |
| | | CTGAGAAGGA GAGATGAGAG AAAGGTTTGC TTTTATCATT AAAAGGAAAG |
| | | CAGATGGTGG AGCTGAATAT GCCACTTACC AGACTAAATC AACCACTCCA |
| | | GCAGAGCAGA GAGGCTGAAT AGATTCCACA ACCTGGTTTG CCAGTTCATC |
| | | TTTTGACTCT ATTAAAATCT TCAATAGTTG TTATTCTGTA GTTTCACTCT |
| | | CATGAGTGCA ACTGTGGCTT AGCTAATATT GCAATGTGGC TTGAATGTAG |
| | | GTAGCATCCT TTGATGCTTC TTTGAAACTT GTATGAATTT GGGTATGAAC |
| | | AGATTGCCTG CTTTCCCTTA AATAACACTT AGATTTATTG GACCAGTCAG |
| | | CACAGCATGC CTGGTTGTAT TAAAGCAGGG ATATGCTGTA TTTTATAAAA |
| | | TTGGCAAAAT TAGAGAAATA TAGTTCACAA TGAAATTATA TTTTCTTTGT |
| | | AAAGAAAGTG GCTTGAAATC TTTTTTGTTC AAAGATTAAT GCCAACTCTT |
| | | AAGATTATTC TTTCACCAAC TATAGAATGT ATTTTATATA TCGTTCATTG |
| | | TAAAAAGCCC TTAAAAATAT GTGTATACTA CTTTGGCTCT TGTGCATAAA |
| | | AACAAGAACA CTGAAAATTG GGAATATGCA CAAACTTGGC TTCTTTAACC |
| | | AAGAATATTA TTGGAAAATT CTCTAAAAGT TAATAGGGTA AATTCTCTAT |
| | | TTTTTGTAAT GTGTTCGGTG ATTTCAGAAA GCTAGAAAGT GTATGTGTGG |
| | | CATTTGTTTT CACTTTTTAA AACATCCCTA ACTGATCGAA TATATCAGTA |
| | | ATTTCAGAAT CAGATGCATC CTTTCATAAG AAGTGAGAGG ACTCTGACAG |
| | | CCATAACAGG AGTGCCACTT CATGGTGCGA AGTGAACACT GTAGTCTTGT |
| | | TGTTTTCCCA AAGAGAACTC CGTATGTTCT CTTAGGTTGA GTAACCCACT |
| | | CTGAATTCTG GTTACATGTG TTTTTCTCTC CCTCCTTAAA TAAAGAGAGG |
| | | GGTTAAACAT GCCCTCTAAA AGTAGGTGGT TTTGAAGAGA ATAAATTCAT |
| | | CAGATAACCT CAAGTCACAT GAGAATCTTA GTCCATTTAC ATTGCCTTGG |
| | | CTAGTAAAAG CCATCTATGT ATATGTCTTA CCTCATCTCC TAAAAGGCAG |
| | | AGTACAAAGT AAGCCATGTA TCTCAGGAAG GTAACTTCAT TTTGTCTATT |
| | | TGCTGTTGAT TGTACCAAGG GATGGAAGAA GTAAATATAG CTCAGGTAGC |
| | | ACTTTATACT CAGGCAGATC TCAGCCCTCT ACTGAGTCCC TTAGCCAAGC |
| | | AGTTTCTTTC AAAGAAGCCA GCAGGCGAAA AGCAGGGACT GCCACTGCAT |
| | | TTCATATCAC ACTGTTAAAA GTTGTGTTTT GAAATTTTAT GTTTAGTTGC |
| | | ACAAATTGGG CCAAAGAAAC ATTGCCTTGA GGAAGATATG ATTGGAAAAT |
| | | CAAGAGTGTA GAAGAATAAA TACTGTTTTA CTGTCCAAAG ACATGTTTAT |
| | | AGTGCTCTGT AAATGTTCCT TTCCTTTGTA GTCTCTGGCA AGATGCTTTA |
| | | GGAAGATAAA AGTTTGAGGA GAACAAACAG GAATTCTGAA TTAAGCACAG |
| | | AGTTGAAGTT TATACCCGTT TCACATGCTT TTCAAGAATG TCGCAATTAC |
| | | TAAGAAGCAG ATAATGGTGT TTTTTAGAAA CCTAATTGAA GTATATTCAA |
| | | CCAAATACTT TAATGTATAA AATAAATATT ATACAATATA CTTGTATAGC |
| | | AGTTTCTGCT TCACATTTGA TTTTTTCAAA TTTAATATTT ATATTAGAGA |
| | | TCTATATATG TATAAATATG TATTTTGTCA AATTTGTTAC TTAAATATAT |
| | | AGAGACCAGT TTTCTCTGGA AGTTTGTTTA AATGACAGAA GCGTATATGA |
| | | ATTCAAGAAA ATTTAAGCTG CAAAAATGTA TTTGCTATAA AATGAGAAGT |
| | | CTCACTGATA GAGGTTCTTT ATTGCTCATT TTTTAAAAAA TGGACTCTTG |
| | | AAATCTGTTA AAATAAAATT GTACATTTGG AGATGTTTCA TGAAAAAAAA |
| | | AA |
| 36 | CD49e | ATTCGCCTCT GGGAGGTTTA GGAAGCGGCT CCGGGTCGGT GGCCCCAGGA |
| | | CAGGGAAGAG CGGGCGCTAT GGGGAGCCGG ACGCCAGAGT CCCCTCTCCA |
| | | CGCCGTGCAG CTGCGCTGGG GCCCCCGGCG CCGACCCCCG CTGCTGCCGC |
| | | TGCTGTTGCT GCTGCTGCCG CCGCCACCCA GGGTCGGGGG CTTCAACTTA |
| | | GACGCGGAGG CCCCAGCAGT ACTCTCGGGG CCCCCGGGCT CCTTCTTCGG |
| | | ATTCTCAGTG GAGTTTTACC GGCCGGGAAC AGACGGGGTC AGTGTGCTGG |
| | | TGGGAGCACC CAAGGCTAAT ACCAGCCAGC CAGGAGTGCT GCAGGGTGGT |
| | | GCTGTCTACC TCTGTCCTTG GGGTGCCAGC CCCACACAGT GCACCCCCAT |
| | | TGAATTTGAC AGCAAAGGCT CTCGGCTCCT GGAGTCCTCA CTGTCCAGCT |
| | | CAGAGGGAGA GGAGCCTGTG GAGTACAAGT CCTTGCAGTG GTTCGGGGCA |
| | | ACAGTTCGAG CCCATGGCTC CTCCATCTTG GCATGCGCTC CACTGTACAG |
| | | CTGGCGCACA GAGAAGGAGC CACTGAGCGA CCCCGTGGGC ACCTGCTACC |
| | | TCTCCACAGA TAACTTCACC CGAATTCTGG AGTATGCACC CTGCCGCTCA |
| | | GATTTCAGCT GGGCAGCAGG ACAGGGTTAC TGCCAAGGAG GCTTCAGTGC |
| | | CGAGTTCACC AAGACTGGCC GTGTGGTTTT AGGTGGACCA GGAAGCTATT |
| | | TCTGGCAAGG CCAGATCCTG TCTGCCACTC AGGAGCAGAT TGCAGAATCT |
| | | TATTACCCCG AGTACCTGAT CAACCTGGTT CAGGGGCAGC TGCAGACTCG |
| | | CCAGGCCAGT TCCATCTATG ATGACAGCTA CCTAGGATAC TCTGTGGCTG |
| | | TTGGTGAATT CAGTGGTGAT GACACAGAAG ACTTTGTTGC TGGTGTGCCC |
| | | AAAGGGAACC TCACTTACGG CTATGTCACC ATCCTTAATG GCTCAGACAT |
| | | TCGATCCCTC TACAACTTCT CAGGGGAACA GATGGCCTCC TACTTTGGCT |
| | | ATGCAGTGGC CGCCACAGAC GTCAATGGGG ACGGGCTGGA TGACTTGCTG |
| | | GTGGGGGCAC CCCTGCTCAT GGATCGGACC CCTGACGGGC GGCCTCAGGA |
| | | GGTGGGCAGG GTCTACGTCT ACCTGCAGCA CCCAGCCGGC ATAGAGCCCA |
| | | CGCCCACCCT TACCCTCACT GGCCATGATG AGTTTGGCCG ATTTGGCAGC |
| | | TCCTTGACCC CCCTGGGGGA CCTGGACCAG GATGGCTACA ATGATGTGGC |
| | | CATCGGGGCT CCCTTTGGTG GGGAGACCCA GCAGGGAGTA GTGTTTGTAT |
| | | TTCCTGGGGG CCCAGGAGGG CTGGGCTCTA AGCCTTCCCA GGTTCTGCAG |
| | | CCCCTGTGGG CAGCCAGCCA CACCCCAGAC TTCTTTGGCT CTGCCCTTCG |
| | | AGGAGGCCGA GACCTGGATG GCAATGGATA TCCTGATCTG ATTGTGGGGT |
| | | CCTTTGGTGT GGACAAGGCT GTGGTATACA GGGGCCGCCC CATCGTGTCC |
| | | GCTAGTGCCT CCCTCACCAT CTTCCCCGCC ATGTTCAACC CAGAGGAGCG |
| | | GAGCTGCAGC TTAGAGGGGA ACCCTGTGGC CTGCATCAAC CTTAGCTTCT |
| | | GCCTCAATGC TTCTGGAAAA CACGTTGCTG ACTCCATTGG TTTCACAGTG |
| | | GAACTTCAGC TGGACTGGCA GAAGCAGAAG GGAGGGGTAC GGCGGGCACT |
| | | GTTCCTGGCC TCCAGGCAGG CAACCCTGAC CCAGACCCTG CTCATCCAGA |
| | | ATGGGGCTCG AGAGGATTGC AGAGAGATGA AGATCTACCT CAGGAACGAG |
| | | TCAGAATTTC GAGACAAACT CTCGCCGATT CACATCGCTC TCAACTTCTC |
| | | CTTGGACCCC CAAGCCCCAG TGGACAGCCA CGGCCTCAGG CCAGCCCTAC |
| | | ATTATCAGAG CAAGAGCCGG ATAGAGGACA AGGCTCAGAT CTTGCTGGAC |
| | | TGTGGAGAAG ACAACATCTG TGTGCCTGAC CTGCAGCTGG AAGTGTTTGG |
| | | GGAGCAGAAC CATGTGTACC TGGGTGACAA GAATGCCCTG AACCTCACTT |
| | | TCCATGCCCA GAATGTGGGT GAGGGTGGCG CCTATGAGGC TGAGCTTCGG |
| | | GTCACCGCCC CTCCAGAGGC TGAGTACTCA GGACTCGTCA GACACCCAGG |
| | | GAACTTCTCC AGCCTGAGCT GTGACTACTT TGCCGTGAAC CAGAGCCGCC |
| | | TGCTGGTGTG TGACCTGGGC AACCCCATGA AGGCAGGAGC CAGTCTGTGG |
| | | GGTGGCCTTC GGTTTACAGT CCCTCATCTC CGGGACACTA AGAAAACCAT |
| | | CCAGTTTGAC TTCCAGATCC TCAGCAAGAA TCTCAACAAC TCGCAAAGCG |
| | | ACGTGGTTTC CTTTCGGCTC TCCGTGGAGG CTCAGGCCCA GGTCACCCTG |
| | | AACGGTGTCT CCAAGCCTGA GGCAGTGCTA TTCCCAGTAA GCGACTGGCA |
| | | TCCCCGAGAC CAGCCTCAGA AGGAGGAGGA CCTGGGACCT GCTGTCCACC |
| | | ATGTCTATGA GCTCATCAAC CAAGGCCCCA GCTCCATTAG CCAGGGTGTG |
| | | CTGGAACTCA GCTGTCCCCA GGCTCTGGAA GGTCAGCAGC TCCTATATGT |
| | | GACCAGAGTT ACGGGACTCA ACTGCACCAC CAATCACCCC ATTAACCCAA |
| | | AGGGCCTGGA GTTGGATCCC GAGGGTTCCC TGCACCACCA GCAAAAACGG |
| | | GAAGCTCCAA GCCGCAGCTC TGCTTCCTCG GGACCTCAGA TCCTGAAATG |
| | | CCCGGAGGCT GAGTGTTTCA GGCTGCGCTG TGAGCTCGGG CCCCTGCACC |
| | | AACAAGAGAG CCAAAGTCTG CAGTTGCATT TCCGAGTCTG GGCCAAGACT |
| | | TTCTTGCAGC GGGAGCACCA GCCATTTAGC CTGCAGTGTG AGGCTGTGTA |
| | | CAAAGCCCTG AAGATGCCCT ACCGAATCCT GCCTCGGCAG CTGCCCCAAA |
| | | AAGAGCGTCA GGTGGCCACA GCTGTGCAAT GGACCAAGGC AGAAGGCAGC |
| | | TATGGCGTCC CACTGTGGAT CATCATCCTA GCCATCCTGT TTGGCCTCCT |
| | | GCTCCTAGGT CTACTCATCT ACATCCTCTA CAAGCTTGGA TTCTTCAAAC |
| | | GCTCCCTCCC ATATGGCACC GCCATGGAAA AAGCTCAGCT CAAGCCTCCA |
| | | GCCACCTCTG ATGCCTGAGT CCTCCCAATT TCAGACTCCC ATTCCTGAAG |
| | | AACCAGTCCC CCCACCCTCA TTCTACTGAA AAGGAGGGGT CTGGGTACTT |
| | | CTTGAAGGTG CTGACGGCCA GGGAGAAGCT CCTCTCCCCA GCCCAGAGAC |
| | | ATACTTGAAG GGCCAGAGCC AGGGGGGTGA GGAGCTGGGG ATCCCTCCCC |
| | | CCCATGCACT GTGAAGGACC CTTGTTTACA CATACCCTCT TCATGGATGG |
| | | GGGAACTCAG ATCCAGGGAC AGAGGCCCCA GCCTCCCTGA AGCCTTTGCA |
| | | TTTTGGAGAG TTTCCTGAAA CAACTTGGAA AGATAACTAG GAAATCCATT |
| | | CACAGTTCTT TGGGCCAGAC ATGCCACAAG GACTTCCTGT CCAGCTCCAA |
| | | CCTGCAAAGA TCTGTCCTCA GCCTTGCCAG AGATCCAAAA GAAGCCCCCA |
| | | GCTAAGAACC TGGAACTTGG GGAGTTAAGA CCTGGCAGCT CTGGACAGCC |
| | | CCACCCTGGT GGGCCAACAA AGAACACTAA CTATGCATGG TGCCCCAGGA |
| | | CCAGCTCAGG ACAGATGCCA CACAAGGATA GATGCTGGCC CAGGGCCCAG |
| | | AGCCCAGCTC CAAGGGGAAT CAGAACTCAA ATGGGGCCAG ATCCAGCCTG |
| | | GGGTCTGGAG TTGATCTGGA ACCCAGACTC AGACATTGGC ACCTAATCCA |
| | | GGCAGATCCA GGACTATATT TGGGCCTGCT CCAGACCTGA TCCTGGAGGC |
| | | CCAGTTCACC CTGATTTAGG AGAAGCCAGG AATTTCCCAG GACCCTGAAG |
| | | GGGCCATGAT GGCAACAGAT CTGGAACCTC AGCCTGGCCA GACACAGGCC |
| | | CTCCCTGTTC CCCAGAGAAA GGGGAGCCCA CTGTCCTGGG CCTGCAGAAT |
| | | TTGGGTTCTG CCTGCCAGCT GCACTGATGC TGCCCCTCAT CTCTCTGCCC |
| | | AACCCTTCCC TCACCTTGGC ACCAGACACC CAGGACTTAT TTAAACTCTG |
| | | TTGCAAGTGC AATAAATCTG ACCCAGTGCC CCCACTGACC AGAACTAGAA |
| 37 | CD81 | GGCCAGAGAG CGAGCGCGCA ACGGCGGCGA CGGCGGCGAC CCCACCGCGC |
| | | ATCCTGCCAG GCCTCCGGCG CCCAGCGCCC CACGCGCCCC CGCGCCCCCG |
| | | CGCCCCCGCG CCCCTTTCTT CGCGCCCCCG CCCCTCGGCC CGCCAGGCCC |
| | | CCTTGCCGGC CACCCGCCAG GCCCCGCGCC GGCCCGCCCG CCGCCCAGGA |
| | | CCGGCCCGCG CCCCGCAGGC CGCCCGCCGC CCGCGCCGCC ATGGGAGTGG |
| | | AGGGCTGCAC CAAGTGCATC AAGTACCTGC TCTTCGTCTT CAATTTCGTC |
| | | TTCTGGCTGG CTGGAGGCGT GATCCTGGGT GTGGCCCTGT GGCTCCGCCA |
| | | TGACCCGCAG ACCACCAACC TCCTGTATCT GGAGCTGGGA GACAAGCCCG |
| | | CGCCCAACAC CTTCTATGTA GGCATCTACA TCCTCATCGC TGTGGGCGCT |
| | | GTCATGATGT TCGTTGGCTT CCTGGGCTGC TACGGGGCCA TCCAGGAATC |
| | | CCAGTGCCTG CTGGGGACGT TCTTCACCTG CCTGGTCATC CTGTTTGCCT |
| | | GTGAGGTGGC CGCCGGCATC TGGGGCTTTG TCAACAAGGA CCAGATCGCC |
| | | AAGGATGTGA AGCAGTTCTA TGACCAGGCC CTACAGCAGG CCGTGGTGGA |
| | | TGATGACGCC AACAACGCCA AGGCTGTGGT GAAGACCTTC CACGAGACGC |
| | | TTGACTGCTG TGGCTCCAGC ACACTGACTG CTTTGACCAC CTCAGTGCTC |
| | | AAGAACAATT TGTGTCCCTC GGGCAGCAAC ATCATCAGCA ACCTCTTCAA |
| | | GGAGGACTGC CACCAGAAGA TCGATGACCT CTTCTCCGGG AAGCTGTACC |
| | | TCATCGGCAT TGCTGCCATC GTGGTCGCTG TGATCATGAT CTTCGAGATG |
| | | ATCCTGAGCA TGGTGCTGTG CTGTGGCATC CGGAACAGCT CCGTGTACTG |
| | | AGGCCCCGCA GCTCTGGCCA CAGGGACCTC TGCAGTGCCC CCTAAGTGAC |
| | | CCGGACACTT CCGAGGGGGC CATCACCGCC TGTGTATATA ACGTTTCCGG |
| | | TATTACTCTG CTACACGTAG CCTTTTTACT TTTGGGGTTT TGTTTTTGTT |
| | | CTGAACTTTC CTGTTACCTT TTCAGGGCTG ACGTCACATG TAGGTGGCGT |
| | | GTATGAGTGG AGACGGGCCT GGGTCTTGGG GACTGGAGGG CAGGGGTCCT |
| | | TCTGCCCTGG GGTCCCAGGG TGCTCTGCCT GCTCAGCCAG GCCTCTCCTG |
| | | GGAGCCACTC GCCCAGAGAC TCAGCTTGGC CAACTTGGGG GGCTGTGTCC |
| | | ACCCAGCCCG CCCGTCCTGT GGGCTGCACA GCTCACCTTG TTCCCTCCTG |
| | | CCCCGGTTCG AGAGCCGAGT CTGTGGGCAC TCTCTGCCTT CATGCACCTG |
| | | TCCTTTCTAA CACGTCGCCT TCAACTGTAA TCACAACATC CTGACTCCGT |
| | | CATTTAATAA AGAAGGAACA TCAGGCATGC TA |
| 38 | CD146 | ACTTGGCTCT CGCCCTCCGG CCGGGAAGCA TGGGGCTTCC CAGGCTGGTC |
| | | TGCGCCTTCT TGCTCGCCGC CTGCTGCTGC TGTCCTCGCG TCGCGGGTGT |
| | | GCCCGGAGAG GCTGAGCAGC CTGCGCCTGA GCTGGTGGAG GTGGAAGTGG |
| | | GCAGCACAGC CCTTCTGAAG TGCGGCCTCT CCCAGTCCCA AGGCAACCTC |
| | | AGCCATGTCG ACTGGTTTTC TGTCCACAAG GAGAAGCGGA CGCTCATCTT |
| | | CCGTGTGCGC CAGGGCCAGG GCCAGAGCGA ACCTGGGGAG TACGAGCAGC |
| | | GGCTCAGCCT CCAGGACAGA GGGGCTACTC TGGCCCTGAC TCAAGTCACC |
| | | CCCCAAGACG AGCGCATCTT CTTGTGCCAG GGCAAGCGCC CTCGGTCCCA |
| | | GGAGTACCGC ATCCAGCTCC GCGTCTACAA AGCTCCGGAG GAGCCAAACA |
| | | TCCAGGTCAA CCCCCTGGGC ATCCCTGTGA ACAGTAAGGA GCCTGAGGAG |
| | | GTCGCTACCT GTGTAGGGAG GAACGGGTAC CCCATTCCTC AAGTCATCTG |
| | | GTACAAGAAT GGCCGGCCTC TGAAGGAGGA GAAGAACCGG GTCCACATTC |
| | | AGTCGTCCCA GACTGTGGAG TCGAGTGGTT TGTACACCTT GCAGAGTATT |
| | | CTGAAGGCAC AGCTGGTTAA AGAAGACAAA GATGCCCAGT TTTACTGTGA |
| | | GCTCAACTAC CGGCTGCCCA GTGGGAACCA CATGAAGGAG TCCAGGGAAG |
| | | TCACCGTCCC TGTTTTCTAC CCGACAGAAA AAGTGTGGCT GGAAGTGGAG |
| | | CCCGTGGGAA TGCTGAAGGA AGGGGACCGC GTGGAAATCA GGTGTTTGGC |
| | | TGATGGCAAC CCTCCACCAC ACTTCAGCAT CAGCAAGCAG AACCCCAGCA |
| | | CCAGGGAGGC AGAGGAAGAG ACAACCAACG ACAACGGGGT CCTGGTGCTG |
| | | GAGCCTGCCC GGAAGGAACA CAGTGGGCGC TATGAATGTC AGGGCCTGGA |
| | | CTTGGACACC ATGATATCGC TGCTGAGTGA ACCACAGGAA CTACTGGTGA |
| | | ACTATGTGTC TGACGTCCGA GTGAGTCCCG CAGCCCCTGA GAGACAGGAA |
| | | GGCAGCAGCC TCACCCTGAC CTGTGAGGCA GAGAGTAGCC AGGACCTCGA |
| | | GTTCCAGTGG CTGAGAGAAG AGACAGGCCA GGTGCTGGAA AGGGGGCCTG |
| | | TGCTTCAGTT GCATGACCTG AAACGGGAGG CAGGAGGCGG CTATCGCTGC |
| | | GTGGCGTCTG TGCCCAGCAT ACCCGGCCTG AACCGCACAC AGCTGGTCAA |
| | | CGTGGCCATT TTTGGCCCCC CTTGGATGGC ATTCAAGGAG AGGAAGGTGT |
| | | GGGTGAAAGA GAATATGGTG TTGAATCTGT CTTGTGAAGC GTCAGGGCAC |
| | | CCCCGGCCCA CCATCTCCTG GAACGTCAAC GGCACGGCAA GTGAACAAGA |
| | | CCAAGATCCA CAGCGAGTCC TGAGCACCCT GAATGTCCTC GTGACCCCGG |
| | | AGCTGTTGGA GACAGGTGTT GAATGCACGG CCTCCAACGA CCTGGGCAAA |
| | | AACACCAGCA TCCTCTTCCT GGAGCTGGTC AATTTAACCA CCCTCACACC |
| | | AGACTCCAAC ACAACCACTG GCCTCAGCAC TTCCACTGCC AGTCCTCATA |
| | | CCAGAGCCAA CAGCACCTCC ACAGAGAGAA AGCTGCCGGA GCCGGAGAGC |
| | | CGGGGCGTGG TCATCGTGGC TGTGATTGTG TGCATCCTGG TCCTGGCGGT |
| | | GCTGGGCGCT GTCCTCTATT TCCTCTATAA GAAGGGCAAG CTGCCGTGCA |
| | | GGCGCTCAGG GAAGCAGGAG ATCACGCTAC CCCCGTCTCG TAAGAGCGAA |
| | | CTTGTAGTTG AAGTTAAGTC AGATAAGCTC CCAGAAGAGA TGGGCCTCCT |
| | | GCAGGGCAGC AGCGGTGACA AGAGGGCTCC GGGAGACCAG GGAGAGAAAT |
| | | ACATCGATCT GAGGCATTAG CCCCGAATCA CTTCAGCTCC CTTCCCTGCC |
| | | TGGACCATTC CCAGCTCCCT GCTCACTCTT CTCTCAGCCA AAGCCTCCAA |
| | | AGGGACTAGA GAGAAGCCTC CTGCTCCCCT CGCCTGCACA CCCCCTTTCA |
| | | GAGGGCCACT GGGTTAGGAC CTGAGGACCT CACTTGGCCC TGCAAGGCCC |
| | | GCTTTTCAGG GACCAGTCCA CCACCATCTC CTCCACGTTG AGTGAAGCTC |
| | | ATCCCAAGCA AGGAGCCCCA GTCTCCCGAG CGGGTAGGAG AGTTTCTTGC |
| | | AGAACGTGTT TTTTCTTTAC ACACATTATG GCTGTAAATA CCTGGCTCCT |
| | | GCCAGCAGCT GAGCTGGGTA GCCTCTCTGA GCTGGTTTCC TGCCCCAAAG |
| | | GCTGGCTTCC ACCATCCAGG TGCACCACTG AAGTGAGGAC ACACCGGAGC |
| | | CAGGCGCCTG CTCATGTTGA AGTGCGCTGT TCACACCCGC TCCGGAGAGC |
| | | ACCCCAGCAG CATCCAGAAG CAGCTGCAGT GTTGCTGCCA CCACCCTCCT |
| | | GTCTGCCTCT TCAAAGTCTC CTGTGACATT TTTTCTTTGG TCAGAAGCCA |
| | | GGAACTGGTG TCATTCCTTA AAAGATACGT GCCGGGGCCA GGTGTGGTGG |
| | | CTCACGCCTG TAATCCCAGC ACTTTGGGAG GCCGAGGCGG GCGGATCACA |
| | | AAGTCAGGAC GAGACCATCC TGGCTAACAC GGTGAAACCC TGTCTCTACT |
| | | AAAAATACAA AAAAAAATTA GCTAGGCGTA GTGGTTGGCA CCTATAGTCC |
| | | CAGCTACTCG GAAGGCTGAA GCAGGAGAAT GGTATGAATC CAGGAGGTGG |
| | | AGCTTGCAGT GAGCCGAGAC CGTGCCACTG CACTCCAGCC TGGGCAACAC |
| | | AGCGAGACTC CGTCTCGAGG AAAAAAAAAG AAAAGATACG TGCCTGCGGT |
| | | GAGGAAGCTG GGCGCTGTTT TCGAGTTCAG GTGAATTAGC CTCAATCCCC |
| | | CGTGTTCACT TGGCTCCCAT AGCCCTCTTG ATGGATCACG TAAAACTGAA |
| | | AGGCAGCGGG GAGCAGACAA AGATGAGGTC TACACTGTCC TTCATGGGGA |
| | | TTAAAGCTAT GGTTATATTA GCACCAAACT TCTACAAACC AAGCTCAGGG |
| | | CCCCAACCCT AGAAGGGCCC AAATGAGAGA ATGGTACTTA GGGATGGAAA |
| | | ACGGGCCTGG CTAGAGCTTC GGGTGTGTGT GTCTGTCTGT GTGTATGCAT |
| | | ACATATGTGT GTATATATGG TTTTGTCAGG TGTGTAAATT TGCAAATTGT |
| | | TTCCTTTATA TATGTATGTA TATATATATA TGAAAATATA TATATATATG |
| | | AAAAATAAAG CTTAATTGTC CCAGAAA |
| 39 | CD147 | AGCGGTTGGA GGTTGTAGGA CCGGCGAGGA ATAGGAATCA TGGCGGCTGC |
| | | GCTGTTCGTG CTGCTGGGAT TCGCGCTGCT GGGCACCCAC GGAGCCTCCG |
| | | GGGCTGCCGG CACAGTCTTC ACTACCGTAG AAGACCTTGG CTCCAAGATA |
| | | CTCCTCACCT GCTCCTTGAA TGACAGCGCC ACAGAGGTCA CAGGGCACCG |
| | | CTGGCTGAAG GGGGGCGTGG TGCTGAAGGA GGACGCGCTG CCCGGCCAGA |
| | | AAACGGAGTT CAAGGTGGAC TCCGACGACC AGTGGGGAGA GTACTCCTGC |
| | | GTCTTCCTCC CCGAGCCCAT GGGCACGGCC AACATCCAGC TCCACGGGCC |
| | | TCCCAGAGTG AAGGCTGTGA AGTCGTCAGA ACACATCAAC GAGGGGGAGA |
| | | CGGCCATGCT GGTCTGCAAG TCAGAGTCCG TGCCACCTGT CACTGACTGG |
| | | GCCTGGTACA AGATCACTGA CTCTGAGGAC AAGGCCCTCA TGAACGGCTC |
| | | CGAGAGCAGG TTCTTCGTGA GTTCCTCGCA GGGCCGGTCA GAGCTACACA |
| | | TTGAGAACCT GAACATGGAG GCCGACCCCG GCCAGTACCG GTGCAACGGC |
| | | ACCAGCTCCA AGGGCTCCGA CCAGGCCATC ATCACGCTCC GCGTGCGCAG |
| | | CCACCTGGCC GCCCTCTGGC CCCTCCTGGG CATCGTGGCT GAGGTGCTGG |
| | | TGCTGGTCAC CATCATCTTC ATCTACGAGA AGCGCCGGAA GCCCGAGGAC |
| | | GTCCTGGATG ATGACGACGC CGGCTCTGCA CCCCTGAAGA GCAGCGGGCA |
| | | GCACCAGAAT GACAAAGGCA AGAACGTCCG CCAGAGGAAC TCTTCCTGAG |
| | | GCAGGTGGCC CGAGGACGCT CCCTGCTCCA CGTCTGCGCC GCCGCCGGAG |
| | | TCCACTCCCA GTGCTTGCAA GATTCCAAGT TCTCACCTCT TAAAGAAAAC |
| | | CCACCCCGTA GATTCCCATC AT |
| 40 | CD166 | AGAGCAGCCC GGAGACCGCT GCCGCCGCTG CCGCTGCTAC CACCGCTGCC |
| | | ACCTGAGGAG ACCCGCCGCC CCCCCGTCGC CGCCTCCTGC GAGTCCTTCT |
| | | TAGCACCTGG CGTTTCATGC ACATTGCCAC TGCCATTATT ATTATCATTC |
| | | CAATACAAGG AAAATAAAAG AAGATACCAG CGAAAAGAAC CGCTTACACC |
| | | TTTCCGAATT ACTCAAGTGT CTCCTGGAAA CAGAGGGTCG TTGTCCCCGG |
| | | AGGAGCAGCC GAAGGGCCCG TGGGCTGGTG TTGACCGGGA GGGAGGAGGA |
| | | GTTGGGGGCA TTGCGTGGTG GAAAGTTGCG TGCGGCAGAG AACCGAAGGT |
| | | GCAGCGCCAC AGCCCAGGGG ACGGTGTGTC TGGGAGAAGA CGCTGCCCCT |
| | | GCGTCGGGAC CCGCCAGCGC GCGGGCACCG CGGGGCCCGG GACGACGCCC |
| | | CCTCCTGCGG CGTGGACTCC GTCAGTGGCC CACCAAGAAG GAGGAGGAAT |
| | | ATGGAATCCA AGGGGGCCAG TTCCTGCCGT CTGCTCTTCT GCCTCTTGAT |
| | | CTCCGCCACC GTCTTCAGGC CAGGCCTTGG ATGGTATACT GTAAATTCAG |
| | | CATATGGAGA TACCATTATC ATACCTTGCC GACTTGACGT ACCTCAGAAT |
| | | CTCATGTTTG GCAAATGGAA ATATGAAAAG CCCGATGGCT CCCCAGTATT |
| | | TATTGCCTTC AGATCCTCTA CAAAGAAAAG TGTGCAGTAC GACGATGTAC |
| | | CAGAATACAA AGACAGATTG AACCTCTCAG AAAACTACAC TTTGTCTATC |
| | | AGTAATGCAA GGATCAGTGA TGAAAAGAGA TTTGTGTGCA TGCTAGTAAC |
| | | TGAGGACAAC GTGTTTGAGG CACCTACAAT AGTCAAGGTG TTCAAGCAAC |
| | | CATCTAAACC TGAAATTGTA AGCAAAGCAC TGTTTCTCGA AACAGAGCAG |
| | | CTAAAAAAGT TGGGTGACTG CATTTCAGAA GACAGTTATC CAGATGGCAA |
| | | TATCACATGG TACAGGAATG GAAAAGTGCT ACATCCCCTT GAAGGAGCGG |
| | | TGGTCATAAT TTTTAAAAAG GAAATGGACC CAGTGACTCA GCTCTATACC |
| | | ATGACTTCCA CCCTGGAGTA CAAGACAACC AAGGCTGACA TACAAATGCC |
| | | ATTCACCTGC TCGGTGACAT ATTATGGACC ATCTGGCCAG AAAACAATTC |
| | | ATTCTGAACA GGCAGTATTT GATATTTACT ATCCTACAGA GCAGGTGACA |
| | | ATACAAGTGC TGCCACCAAA AAATGCCATC AAAGAAGGGG ATAACATCAC |
| | | TCTTAAATGC TTAGGGAATG GCAACCCTCC CCCAGAGGAA TTTTTGTTTT |
| | | ACTTACCAGG ACAGCCCGAA GGAATAAGAA GCTCAAATAC TTACACACTG |
| | | ACGGATGTGA GGCGCAATGC AACAGGAGAC TACAAGTGTT CCCTGATAGA |
| | | CAAAAAAAGC ATGATTGCTT CAACAGCTAT CACAGTTCAC TATTTGGATT |
| | | TGTCCTTAAA CCCAAGTGGA GAAGTGACTA GACAGATTGG TGATGCCCTA |
| | | CCCGTGTCAT GCACAATATC TGCTAGCAGG AATGCAACTG TGGTATGGAT |
| | | GAAAGATAAC ATCAGGCTTC GATCTAGCCC GTCATTTTCT AGTCTTCATT |
| | | ATCAGGATGC TGGAAACTAT GTCTGCGAAA CTGCTCTGCA GGAGGTTGAA |
| | | GGACTAAAGA AAAGAGAGTC ATTGACTCTC ATTGTAGAAG GCAAACCTCA |
| | | AATAAAAATG ACAAAGAAAA CTGATCCCAG TGGACTATCT AAAACAATAA |
| | | TCTGCCATGT GGAAGGTTTT CCAAAGCCAG CCATTCAATG GACAATTACT |
| | | GGCAGTGGAA GCGTCATAAA CCAAACAGAG GAATCTCCTT ATATTAATGG |
| | | CAGGTATTAT AGTAAAATTA TCATTTCCCC TGAAGAGAAT GTTACATTAA |
| | | CTTGCACAGC AGAAAACCAA CTGGAGAGAA CAGTAAACTC CTTGAATGTC |
| | | TCTGCTAATG AAAACAGAGA AAAGGTGAAT GACCAGGCAA AACTAATTGT |
| | | GGGAATCGTT GTTGGTCTCC TCCTTGCTGC CCTTGTTGCT GGTGTCGTCT |
| | | ACTGGCTGTA CATGAAGAAG TCAAAGACTG CATCAAAACA TGTAAACAAG |
| | | GACCTCGGTA ATATGGAAGA AAACAAAAAG TTAGAAGAAA ACAATCACAA |
| | | AACTGAAGCC TAAGAGAGAA ACTGTCCTAG TTGTCCAGAG ATAAAAATCA |
| | | TATAGACCAA TTGAAGCATG AACGTGGATT GTATTTAAGA CATAAACAAA |
| | | GACATTGACA GCAATTCATG GTTCAAGTAT TAAGCAGTTC ATTCTACCAA |
| | | GCTGTCACAG GTTTTCAGAG AATTATCTCA AGTAAAACAA ATGAAATTTA |
| | | ATTACAAACA ATAAGAACAA GTTTTGGCAG CCATGATAAT AGGTCATATG |
| | | TTGTGTTTGG TTCAATTTTT TTTCCGTAAA TGTCTGCACT GAGGATTTCT |
| | | TTTTGGTTTG CCTTTTATGT AAATTTTTTA CGTAGCTATT TTTATACACT |
| | | GTAAGCTTTG TTCTGGGAGT TGCTGTTAAT CTGATGTATA ATGTAATGTT |
| | | TTTATTTCAA TTGTTTATAT GGATAATCTG AGCAGGTACA TTTCTGATTC |
| | | TGATTGCTAT CAGCAATGCC CCAAACTTTC TCATAAGCAC CTAAAACCCA |
| | | AAGGTGGCAG CTTGTGAAGA TTGGGGACAC TCATATTGCC CTAATTAAAA |
| | | ACTGTGATTT TTATCACAAG GGAGGGGAGG CCGAGAGTCA GACTGATAGA |
| | | CACCATAGGA GCCGACTCTT TGATATGCCA CCAGCGAACT CTCAGAAATA |
| | | AATCACAGAT GCATATAGAC ACACATACAT AATGGTACTC CCAAACTGAC |
| | | AATTTTACCT ATTCTGAAAA AGACATAAAA CAGAATTTGG TAGCACTTAC |
| | | CTCTACAGAC ACCTGCTAAT AAATTATTTT CTGTCAAAAG AAAAAACACA |
| | | AGCATGTGTG AGAGACAGTT TGGAAAAATC ATGGTCAACA TTCCCATTTT |
| | | CATAGATCAC AATGTAAATC ACTATAATTA CAAATTGGTG TTAAATCCTT |
| | | TGGGTTATCC ACTGCCTTAA AATTATACCT ATTTCATGTT TAAAAAGATA |
| | | TCAATCAGAA TTGGAGTTTT TAACAGTGGT CATTATCAAA GCTGTGTTAT |
| | | TTTCCACAGA ATATAGAATA TATATTTTTT TCGTGTGTGT TTTTGTTAAC |
| | | TACCCTACAG ATATTGAATG CACCTTGAGA TAATTTAGTG TTTTTAACTG |
| | | ATACATAATT TATCAAGCAG TACATGAAAG TGTAATAATA AAATGTCTAT |
| | | GTATCTTTAG TTACATTCAA ATTTGTAACT TTATAAACAT GTTTTATGCT |
| | | TGAGGAAATT TTTAAGGTGG TAGTATAAAT GGAAACTTTT TGAAGTAGAC |
| | | CAGATATGGG CTACTTGTGA CTAGACTTTT AAACTTTGCT CTTTCAAGCA |
| | | GAAGCCTGGT TTCTGGGAGA ACACTGCACA GCGATTTCTT TCCCAGGATT |
| | | TACACAACTT TAAAGGGAAG ATAAATGAAC ATCAGATTTC TAGGTATAGA |
| | | ACTATGTTAT TGAAAGGAAA AGGAAAACTG GTGTTTGTTT CTTAGACTCA |
| | | TGAAATAAAA AATTATGAAG GCAATGAAAA ATAAATTGAA AATTAAAGTC |
| | | AGATGAGAAT AGGAATAATA CTTTGCCACT TCTGCATTAT TTAGAAACAT |
| | | ACGTTATTGT ACATTTGTAA ACCATTTACT GTCTGGGCAA TAGTGACTCC |
| | | GTTTAATAAA AGCTTCCGTA GTGCATTGGT ATGGATTAAA TGCATAAAAT |
| | | ATTCTTAGAC TCGATGCTGT ATAAAATATT ATGGGAAAAA AAGAAAATAC |
| | | GTTATTTTGC CTCTAAACTT TTATTGAAGT TTTATTTGGC AGGAAAAAAA |
| | | ATTGAATCTT GGTCAACATT TAAACCAAAG TAAAAGGGGA AAAACCAAAG |
| | | TTATTTGTTT TGCATGGCTA AGCCATTCTG TTATCTCTGT AAATACTGTG |
| | | ATTTCTTTTT TATTTTCTCT TTAGAATTTT GTTAAAGAAA TTCTAAAATT |
| | | TTTAAACACC TGCTCTCCAC AATAAATCAC AAACACTAAA ATAAAATTAC |
| | | TTCCATATAA ATATTATTTT CTCTTTTGGT GTGGGAGATC AAAGGTTTAA |
| | | AGTCTAACTT CTAAGATATA TTTGCAGAAA GAAGCAACAT GACAATAGAG |
| | | AGAGTTATGC TACAATTATT TCTTGGTTTC CACTTGCAAT GGTTAATTAA |
| | | GTCCAAAAAC AGCTGTCAGA ACCTCGAGAG CAGAACATGA GAAACTCAGA |
| | | GCTCTGGACC GAAAGCAGAA AGTTTGCCGG GAAAAAAAAA GACAACATTA |
| | | TTACCATCGA TTCAGTGCCT GGATAAAGAG GAAAGCTTAC TTGTTTAATG |
| | | GCAGCCACAT GCACGAAGAT GCTAAGAAGA AAAAGAATTC CAAATCCTCA |
| | | ACTTTTGAGG TTTCGGCTCT CCAATTTAAC TCTTTGGCAA CAGGAAACAG |
| | | GTTTTGCAAG TTCAAGGTTC ACTCCCTATA TGTGATTATA GGAATTGTTT |
| | | GTGGAAATGG ATTAACATAC CCGTCTATGC CTAAAAGATA ATAAAACTGA |
| | | AATATGTCTT CA |
| 41 | CD171 | AGTCACTAAC GTCCTTCCGT TCTCTCGTCT CTCTTCCCCC ACCCTTCCCT |
| | | TCCCTTCTCC CCTCTCCCAG TGCCCCCACT CCCAACTCCC GCCCCAAGCC |
| | | GCCCACCAGC CCCCTTCCCC TCCGGCCGGA GCCTGAACCG AGCCCGGCTG |
| | | GCTGTGCTGC GCGGTGCCGC CGGGAAAGAT GGTCGTGGCG CTGCGGTACG |
| | | TGTGGCCTCT CCTCCTCTGC AGCCCCTGCC TGCTTATCCA GATCCCCGAG |
| | | GAATATGAAG GACACCATGT GATGGAGCCA CCTGTCATCA CGGAACAGTC |
| | | TCCACGGCGC CTGGTTGTCT TCCCCACAGA TGACATCAGC CTCAAGTGTG |
| | | AGGCCAGTGG CAAGCCCGAA GTGCAGTTCC GCTGGACGAG GGATGGTGTC |
| | | CACTTCAAAC CCAAGGAAGA GCTGGGTGTG ACCGTGTACC AGTCGCCCCA |
| | | CTCTGGCTCC TTCACCATCA CGGGCAACAA CAGCAACTTT GCTCAGAGGT |
| | | TCCAGGGCAT CTACCGCTGC TTTGCCAGCA ATAAGCTGGG CACCGCCATG |
| | | TCCCATGAGA TCCGGCTCAT GGCCGAGGGT GCCCCCAAGT GGCCAAAGGA |
| | | GACAGTGAAG CCCGTGGAGG TGGAGGAAGG GGAGTCAGTG GTTCTGCCTT |
| | | GCAACCCTCC CCCAAGTGCA GAGCCTCTCC GGATCTACTG GATGAACAGC |
| | | AAGATCTTGC ACATCAAGCA GGACGAGCGG GTGACGATGG GCCAGAACGG |
| | | CAACCTCTAC TTTGCCAATG TGCTCACCTC CGACAACCAC TCAGACTACA |
| | | TCTGCCACGC CCACTTCCCA GGCACCAGGA CCATCATTCA GAAGGAACCC |
| | | ATTGACCTCC GGGTCAAGGC CACCAACAGC ATGATTGACA GGAAGCCGCG |
| | | CCTGCTCTTC CCCACCAACT CCAGCAGCCA CCTGGTGGCC TTGCAGGGGC |
| | | AGCCATTGGT CCTGGAGTGC ATCGCCGAGG GCTTTCCCAC GCCCACCATC |
| | | AAATGGCTGC GCCCCAGTGG CCCCATGCCA GCCGACCGTG TCACCTACCA |
| | | GAACCACAAC AAGACCCTGC AGCTGCTGAA AGTGGGCGAG GAGGATGATG |
| | | GCGAGTACCG CTGCCTGGCC GAGAACTCAC TGGGCAGTGC CCGGCATGCG |
| | | TACTATGTCA CCGTGGAGGC TGCCCCGTAC TGGCTGCACA AGCCCCAGAG |
| | | CCATCTATAT GGGCCAGGAG AGACTGCCCG CCTGGACTGC CAAGTCCAGG |
| | | GCAGGCCCCA ACCAGAGGTC ACCTGGAGAA TCAACGGGAT CCCTGTGGAG |
| | | GAGCTGGCCA AAGACCAGAA GTACCGGATT CAGCGTGGCG CCCTGATCCT |
| | | GAGCAACGTG CAGCCCAGTG ACACAATGGT GACCCAATGT GAGGCCCGCA |
| | | ACCGGCACGG GCTCTTGCTG GCCAATGCCT ACATCTACGT TGTCCAGCTG |
| | | CCAGCCAAGA TCCTGACTGC GGACAATCAG ACGTACATGG CTGTCCAGGG |
| | | CAGCACTGCC TACCTTCTGT GCAAGGCCTT CGGAGCGCCT GTGCCCAGTG |
| | | TTCAGTGGCT GGACGAGGAT GGGACAACAG TGCTTCAGGA CGAACGCTTC |
| | | TTCCCCTATG CCAATGGGAC CCTGGGCATT CGAGACCTCC AGGCCAATGA |
| | | CACCGGACGC TACTTCTGCC TGGCTGCCAA TGACCAAAAC AATGTTACCA |
| | | TCATGGCTAA CCTGAAGGTT AAAGATGCAA CTCAGATCAC TCAGGGGCCC |
| | | CGCAGCACAA TCGAGAAGAA AGGTTCCAGG GTGACCTTCA CGTGCCAGGC |
| | | CTCCTTTGAC CCCTCCTTGC AGCCCAGCAT CACCTGGCGT GGGGACGGTC |
| | | GAGACCTCCA GGAGCTTGGG GACAGTGACA AGTACTTCAT AGAGGATGGG |
| | | CGCCTGGTCA TCCACAGCCT GGACTACAGC GACCAGGGCA ACTACAGCTG |
| | | CGTGGCCAGT ACCGAACTGG ATGTGGTGGA GAGTAGGGCA CAGCTCTTGG |
| | | TGGTGGGGAG CCCTGGGCCG GTGCCACGGC TGGTGCTGTC CGACCTGCAC |
| | | CTGCTGACGC AGAGCCAGGT GCGCGTGTCC TGGAGTCCTG CAGAAGACCA |
| | | CAATGCCCCC ATTGAGAAAT ATGACATTGA ATTTGAGGAC AAGGAAATGG |
| | | CGCCTGAAAA ATGGTACAGT CTGGGCAAGG TTCCAGGGAA CCAGACCTCT |
| | | ACCACCCTCA AGCTGTCGCC CTATGTCCAC TACACCTTTA GGGTTACTGC |
| | | CATAAACAAA TATGGCCCCG GGGAGCCCAG CCCGGTCTCT GAGACTGTGG |
| | | TCACACCTGA GGCAGCCCCA GAGAAGAACC CTGTGGATGT GAAGGGGGAA |
| | | GGAAATGAGA CCACCAATAT GGTCATCACG TGGAAGCCGC TCCGGTGGAT |
| | | GGACTGGAAC GCCCCCCAGG TTCAGTACCG CGTGCAGTGG CGCCCTCAGG |
| | | GGACACGAGG GCCCTGGCAG GAGCAGATTG TCAGCGACCC CTTCCTGGTG |
| | | GTGTCCAACA CGTCCACCTT CGTGCCCTAT GAGATCAAAG TCCAGGCCGT |
| | | CAACAGCCAG GGCAAGGGAC CAGAGCCCCA GGTCACTATC GGCTACTCTG |
| | | GAGAGGACTA CCCCCAGGCA ATCCCTGAGC TGGAAGGCAT TGAAATCCTC |
| | | AACTCAAGTG CCGTGCTGGT CAAGTGGCGG CCGGTGGACC TGGCCCAGGT |
| | | CAAGGGCCAC CTCCGCGGAT ACAATGTGAC GTACTGGAGG GAGGGCAGTC |
| | | AGAGGAAGCA CAGCAAGAGA CATATCCACA AAGACCATGT GGTGGTGCCC |
| | | GCCAACACCA CCAGTGTCAT CCTCAGTGGC TTGCGGCCCT ATAGCTCCTA |
| | | CCACCTGGAG GTGCAGGCCT TTAACGGGCG AGGATCGGGG CCCGCCAGCG |
| | | AGTTCACCTT CAGCACCCCA GAGGGAGTGC CTGGCCACCC CGAGGCGTTG |
| | | CACCTGGAGT GCCAGTCGAA CACCAGCCTG CTGCTGCGCT GGCAGCCCCC |
| | | ACTCAGCCAC AACGGCGTGC TCACCGGCTA CGTGCTCTCC TACCACCCCC |
| | | TGGATGAGGG GGGCAAGGGG CAACTGTCCT TCAACCTTCG GGACCCCGAA |
| | | CTTCGGACAC ACAACCTGAC CGATCTCAGC CCCCACCTGC GGTACCGCTT |
| | | CCAGCTTCAG GCCACCACCA AAGAGGGCCC TGGTGAAGCC ATCGTACGGG |
| | | AAGGAGGCAC TATGGCCTTG TCTGGGATCT CAGATTTTGG CAACATCTCA |
| | | GCCACAGCGG GTGAAAACTA CAGTGTCGTC TCCTGGGTCC CCAAGGAGGG |
| | | CCAGTGCAAC TTCAGGTTCC ATATCTTGTT CAAAGCCTTG GGAGAAGAGA |
| | | AGGGTGGGGC TTCCCTTTCG CCACAGTATG TCAGCTACAA CCAGAGCTCC |
| | | TACACGCAGT GGGACCTGCA GCCTGACACT GACTACGAGA TCCACTTGTT |
| | | TAAGGAGAGG ATGTTCCGGC ACCAAATGGC TGTGAAGACC AATGGCACAG |
| | | GCCGCGTGAG GCTCCCTCCT GCTGGCTTCG CCACTGAGGG CTGGTTCATC |
| | | GGCTTTGTGA GTGCCATCAT CCTCCTGCTC CTCGTCCTGC TCATCCTCTG |
| | | CTTCATCAAG CGCAGCAAGG GCGGCAAATA CTCAGTGAAG GATAAGGAGG |
| | | ACACCCAGGT GGACTCTGAG GCCCGACCGA TGAAAGATGA GACCTTCGGC |
| | | GAGTACAGGT CCCTGGAGAG TGACAACGAG GAGAAGGCCT TTGGCAGCAG |
| | | CCAGCCATCG CTCAACGGGG ACATCAAGCC CCTGGGCAGT GACGACAGCC |
| | | TGGCCGATTA TGGGGGCAGC GTGGATGTTC AGTTCAACGA GGATGGTTCG |
| | | TTCATTGGCC AGTACAGTGG CAAGAAGGAG AAGGAGGCGG CAGGGGGCAA |
| | | TGACAGCTCA GGGGCCACTT CCCCCATCAA CCCTGCCGTG GCCCTAGAAT |
| | | AGTGGAGTCC AGGACAGGAG ATGCTGTGCC CCTGGCCTTG GGATCCAGGC |
| | | CCCTCCCTCT CCAGCAGGCC CATGGGAGGC TGGAGTTGGG GCAGAGGAGA |
| | | ACTTGCTGCC TCGGATCCCC TTCCTACCAC CCGGTCCCCA CTTTATTGCC |
| | | AAAACCCAGC TGCACCCCTT CCTGGGCACA CGCTGCTCTG CCCCAGCTTG |
| | | GGCAGATCTC CCACATGCCA GGGGCCTTTG GGTGCTGTTT TGCCAGCCCA |
| | | TTTGGGCAGA GAGGCTGTGG TTTGGGGGAG AAGAAGTAGG GGTGGCCCGA |
| | | AAGGGTCTCC GAAATGCTGT CTTTCTTGCT CCCTGACTGG GGGCAGACAT |
| | | GGTGGGGTCT CCTCAGGACC AGGGTTGGCA CCTTCCCCCT CCCCCAGCCA |
| | | CTCCCCAGCC AGCCTGGCTG GGACTGGGAA CAGAACTCGG TGTCCCCACC |
| | | ATCTGCTGTC TTTTCTTTGC CATCTCTGCT CCAACCGGGA TGGGAGCCGG |
| | | GCAAACTGGC CGCGGGGGCA GGGGAGGCCA TCTGGAGAGC CCAGAGTCCC |
| | | CCCACTCCCA GCATCGCACT CTGGCAGCAC CGCCTCTTCC CGCCGCCCAG |
| | | CCCACCCCAT GGCCGGCTTT CAGGAGCTCC ATACACACGC TGCCTTCGGT |
| | | ACCCACCACA CAACATCCAA GTGGCCTCCG TCACTACCTG GCTGCGGGGC |
| | | GGGCACACCT CCTCCCACTG CCCACTGGCC GGCCTCTTCC AGCCGCCCCC |
| | | ACCCCCCAGG ACCCCTTAGC AGCCCTGCCC TCCCTATCGT CTGAACAGTT |
| | | GTCTTCCTCA GCCTCCTCCC GCCCCCACCT TGGGAATGTA AATACACCGT |
| | | GACTTTGAAA GTTTGTACCC CTGTCCTTCC CTTTACGCCA CTAGTGTGTA |
| | | GGCAGATGTC TGAGTCCCTA GGTGGTTTCT AGGATTGATA GCAATTAGCT |
| | | TTGATGAACC CATCCCAGGA AAAATAAAAA CAGACAAAAA AAAAGGAAAG |
| | | ATTGGTTCTC CCAGCACTGC TCAGCAGCCA CAGCCTCCCT GTATGCCTGT |
| | | GCTTGGTCTA CTGATAAGCC CTCTACAAAA AAACAAAAGT ATATATATAT |
| | | ATGTACATAA TATCAGAATT ATAACAGGCA AATAAAACCT GAAAATCAA |
| 42 | ERBB3 | GCAATTTGCA ACCTCCGCTG CCGTCGCCGC AGCAGCCACC AATTCGCCAG |
| | | CGGTTCAGGT GGCTCTTGCC TCGATGTCCT AGCCTAGGGG CCCCCGGGCC |
| | | GGACTTGGCT GGGCTCCCTT CACCCTCTGC GGAGTCATGA GGGCGAACGA |
| | | CGCTCTGCAG GTGCTGGGCT TGCTTTTCAG CCTGGCCCGG GGCTCCGAGG |
| | | TGGGCAACTC TCAGGCAGTG TGTCCTGGGA CTCTGAATGG CCTGAGTGTG |
| | | ACCGGCGATG CTGAGAACCA ATACCAGACA CTGTACAAGC TCTACGAGAG |
| | | GTGTGAGGTG GTGATGGGGA ACCTTGAGAT TGTGCTCACG GGACACAATG |
| | | CCGACCTCTC CTTCCTGCAG TGGATTCGAG AAGTGACAGG CTATGTCCTC |
| | | GTGGCCATGA ATGAATTCTC TACTCTACCA TTGCCCAACC TCCGCGTGGT |
| | | GCGAGGGACC CAGGTCTACG ATGGGAAGTT TGCCATCTTC GTCATGTTGA |
| | | ACTATAACAC CAACTCCAGC CACGCTCTGC GCCAGCTCCG CTTGACTCAG |
| | | CTCACCGAGA TTCTGTCAGG GGGTGTTTAT ATTGAGAAGA ACGATAAGCT |
| | | TTGTCACATG GACACAATTG ACTGGAGGGA CATCGTGAGG GACCGAGATG |
| | | CTGAGATAGT GGTGAAGGAC AATGGCAGAA GCTGTCCCCC CTGTCATGAG |
| | | GTTTGCAAGG GGCGATGCTG GGGTCCTGGA TCAGAAGACT GCCAGACATT |
| | | GACCAAGACC ATCTGTGCTC CTCAGTGTAA TGGTCACTGC TTTGGGCCCA |
| | | ACCCCAACCA GTGCTGCCAT GATGAGTGTG CCGGGGGCTG CTCAGGCCCT |
| | | CAGGACACAG ACTGCTTTGC CTGCCGGCAC TTCAATGACA GTGGAGCCTG |
| | | TGTACCTCGC TGTCCACAGC CTCTTGTCTA CAACAAGCTA ACTTTCCAGC |
| | | TGGAACCCAA TCCCCACACC AAGTATCAGT ATGGAGGAGT TTGTGTAGCC |
| | | AGCTGTCCCC ATAACTTTGT GGTGGATCAA ACATCCTGTG TCAGGGCCTG |
| | | TCCTCCTGAC AAGATGGAAG TAGATAAAAA TGGGCTCAAG ATGTGTGAGC |
| | | CTTGTGGGGG ACTATGTCCC AAAGCCTGTG AGGGAACAGG CTCTGGGAGC |
| | | CGCTTCCAGA CTGTGGACTC GAGCAACATT GATGGATTTG TGAACTGCAC |
| | | CAAGATCCTG GGCAACCTGG ACTTTCTGAT CACCGGCCTC AATGGAGACC |
| | | CCTGGCACAA GATCCCTGCC CTGGACCCAG AGAAGCTCAA TGTCTTCCGG |
| | | ACAGTACGGG AGATCACAGG TTACCTGAAC ATCCAGTCCT GGCCGCCCCA |
| | | CATGCACAAC TTCAGTGTTT TTTCCAATTT GACAACCATT GGAGGCAGAA |
| | | GCCTCTACAA CCGGGGCTTC TCATTGTTGA TCATGAAGAA CTTGAATGTC |
| | | ACATCTCTGG GCTTCCGATC CCTGAAGGAA ATTAGTGCTG GGCGTATCTA |
| | | TATAAGTGCC AATAGGCAGC TCTGCTACCA CCACTCTTTG AACTGGACCA |
| | | AGGTGCTTCG GGGGCCTACG GAAGAGCGAC TAGACATCAA GCATAATCGG |
| | | CCGCGCAGAG ACTGCGTGGC AGAGGGCAAA GTGTGTGACC CACTGTGCTC |
| | | CTCTGGGGGA TGCTGGGGCC CAGGCCCTGG TCAGTGCTTG TCCTGTCGAA |
| | | ATTATAGCCG AGGAGGTGTC TGTGTGACCC ACTGCAACTT TCTGAATGGG |
| | | GAGCCTCGAG AATTTGCCCA TGAGGCCGAA TGCTTCTCCT GCCACCCGGA |
| | | ATGCCAACCC ATGGAGGGCA CTGCCACATG CAATGGCTCG GGCTCTGATA |
| | | CTTGTGCTCA ATGTGCCCAT TTTCGAGATG GGCCCCACTG TGTGAGCAGC |
| | | TGCCCCCATG GAGTCCTAGG TGCCAAGGGC CCAATCTACA AGTACCCAGA |
| | | TGTTCAGAAT GAATGTCGGC CCTGCCATGA GAACTGCACC CAGGGGTGTA |
| | | AAGGACCAGA GCTTCAAGAC TGTTTAGGAC AAACACTGGT GCTGATCGGC |
| | | AAAACCCATC TGACAATGGC TTTGACAGTG ATAGCAGGAT TGGTAGTGAT |
| | | TTTCATGATG CTGGGCGGCA CTTTTCTCTA CTGGCGTGGG CGCCGGATTC |
| | | AGAATAAAAG GGCTATGAGG CGATACTTGG AACGGGGTGA GAGCATAGAG |
| | | CCTCTGGACC CCAGTGAGAA GGCTAACAAA GTCTTGGCCA GAATCTTCAA |
| | | AGAGACAGAG CTAAGGAAGC TTAAAGTGCT TGGCTCGGGT GTCTTTGGAA |
| | | CTGTGCACAA AGGAGTGTGG ATCCCTGAGG GTGAATCAAT CAAGATTCCA |
| | | GTCTGCATTA AAGTCATTGA GGACAAGAGT GGACGGCAGA GTTTTCAAGC |
| | | TGTGACAGAT CATATGCTGG CCATTGGCAG CCTGGACCAT GCCCACATTG |
| | | TAAGGCTGCT GGGACTATGC CCAGGGTCAT CTCTGCAGCT TGTCACTCAA |
| | | TATTTGCCTC TGGGTTCTCT GCTGGATCAT GTGAGACAAC ACCGGGGGGC |
| | | ACTGGGGCCA CAGCTGCTGC TCAACTGGGG AGTACAAATT GCCAAGGGAA |
| | | TGTACTACCT TGAGGAACAT GGTATGGTGC ATAGAAACCT GGCTGCCCGA |
| | | AACGTGCTAC TCAAGTCACC CAGTCAGGTT CAGGTGGCAG ATTTTGGTGT |
| | | GGCTGACCTG CTGCCTCCTG ATGATAAGCA GCTGCTATAC AGTGAGGCCA |
| | | AGACTCCAAT TAAGTGGATG GCCCTTGAGA GTATCCACTT TGGGAAATAC |
| | | ACACACCAGA GTGATGTCTG GAGCTATGGT GTGACAGTTT GGGAGTTGAT |
| | | GACCTTCGGG GCAGAGCCCT ATGCAGGGCT ACGATTGGCT GAAGTACCAG |
| | | ACCTGCTAGA GAAGGGGGAG CGGTTGGCAC AGCCCCAGAT CTGCACAATT |
| | | GATGTCTACA TGGTGATGGT CAAGTGTTGG ATGATTGATG AGAACATTCG |
| | | CCCAACCTTT AAAGAACTAG CCAATGAGTT CACCAGGATG GCCCGAGACC |
| | | CACCACGGTA TCTGGTCATA AAGAGAGAGA GTGGGCCTGG AATAGCCCCT |
| | | GGGCCAGAGC CCCATGGTCT GACAAACAAG AAGCTAGAGG AAGTAGAGCT |
| | | GGAGCCAGAA CTAGACCTAG ACCTAGACTT GGAAGCAGAG GAGGACAACC |
| | | TGGCAACCAC CACACTGGGC TCCGCCCTCA GCCTACCAGT TGGAACACTT |
| | | AATCGGCCAC GTGGGAGCCA GAGCCTTTTA AGTCCATCAT CTGGATACAT |
| | | GCCCATGAAC CAGGGTAATC TTGGGGAGTC TTGCCAGGAG TCTGCAGTTT |
| | | CTGGGAGCAG TGAACGGTGC CCCCGTCCAG TCTCTCTACA CCCAATGCCA |
| | | CGGGGATGCC TGGCATCAGA GTCATCAGAG GGGCATGTAA CAGGCTCTGA |
| | | GGCTGAGCTC CAGGAGAAAG TGTCAATGTG TAGGAGCCGG AGCAGGAGCC |
| | | GGAGCCCACG GCCACGCGGA GATAGCGCCT ACCATTCCCA GCGCCACAGT |
| | | CTGCTGACTC CTGTTACCCC ACTCTCCCCA CCCGGGTTAG AGGAAGAGGA |
| | | TGTCAACGGT TATGTCATGC CAGATACACA CCTCAAAGGT ACTCCCTCCT |
| | | CCCGGGAAGG CACCCTTTCT TCAGTGGGTC TCAGTTCTGT CCTGGGTACT |
| | | GAAGAAGAAG ATGAAGATGA GGAGTATGAA TACATGAACC GGAGGAGAAG |
| | | GCACAGTCCA CCTCATCCCC CTAGGCCAAG TTCCCTTGAG GAGCTGGGTT |
| | | ATGAGTACAT GGATGTGGGG TCAGACCTCA GTGCCTCTCT GGGCAGCACA |
| | | CAGAGTTGCC CACTCCACCC TGTACCCATC ATGCCCACTG CAGGCACAAC |
| | | TCCAGATGAA GACTATGAAT ATATGAATCG GCAACGAGAT GGAGGTGGTC |
| | | CTGGGGGTGA TTATGCAGCC ATGGGGGCCT GCCCAGCATC TGAGCAAGGG |
| | | TATGAAGAGA TGAGAGCTTT TCAGGGGCCT GGACATCAGG CCCCCCATGT |
| | | CCATTATGCC CGCCTAAAAA CTCTACGTAG CTTAGAGGCT ACAGACTCTG |
| | | CCTTTGATAA CCCTGATTAC TGGCATAGCA GGCTTTTCCC CAAGGCTAAT |
| | | GCCCAGAGAA CGTAACTCCT GCTCCCTGTG GCACTCAGGG AGCATTTAAT |
| | | GGCAGCTAGT GCCTTTAGAG GGTACCGTCT TCTCCCTATT CCCTCTCTCT |
| | | CCCAGGTCCC AGCCCCTTTT CCCCAGTCCC AGACAATTCC ATTCAATCTT |
| | | TGGAGGCTTT TAAACATTTT GACACAAAAT TCTTATGGTA TGTAGCCAGC |
| | | TGTGCACTTT CTTCTCTTTC CCAACCCCAG GAAAGGTTTT CCTTATTTTG |
| | | TGTGCTTTCC CAGTCCCATT CCTCAGCTTC TTCACAGGCA CTCCTGGAGA |
| | | TATGAAGGAT TACTCTCCAT ATCCCTTCCT CTCAGGCTCT TGACTACTTG |
| | | GAACTAGGCT CTTATGTGTG CCTTTGTTTC CCATCAGACT GTCAAGAAGA |
| | | GGAAAGGGAG GAAACCTAGC AGAGGAAAGT GTAATTTTGG TTTATGACTC |
| | | TTAACCCCCT AGAAAGACAG AAGCTTAAAA TCTGTGAAGA AAGAGGTTAG |
| | | GAGTAGATAT TGATTACTAT CATAATTCAG CACTTAACTA TGAGCCAGGC |
| | | ATCATACTAA ACTTCACCTA CATTATCTCA CTTAGTCCTT TATCATCCTT |
| | | AAAACAATTC TGTGACATAC ATATTATCTC ATTTTACACA AAGGGAAGTC |
| | | GGGCATGGTG GCTCATGCCT GTAATCTCAG CACTTTGGGA GGCTGAGGCA |
| | | GAAGGATTAC CTGAGGCAAG GAGTTTGAGA CCAGCTTAGC CAACATAGTA |
| | | AGACCCCCAT CTCTTTAAAA AAAAAAAAAA AAAAAAAAAA AAAACTTTAG |
| | | AACTGGGTGC AGTGGCTCAT GCCTGTAATC CCAGCCAGCA CTTTGGGAGG |
| | | CTGAGATGGG AAGATCACTT GAGCCCAGAA TTAGAGATAA GCCTATGGAA |
| | | ACATAGCAAG ACACTGTCTC TACAGGGGAA AAAAAAAAAA GAAACTGAGC |
| | | CTTAAAGAGA TGAAATAAAT TAAGCAGTAG ATCCAGGATG CAAAATCCTC |
| | | CCAATTCCTG TGCATGTGCT CTTATTGTAA GGTGCCAAGA AAAACTGATT |
| | | TAAGTTACAG CCCTTGTTTA AGGGGCACTG TTTCTTGTTT TTGCACTGAA |
| | | TCAAGTCTAA CCCCAACAGC CACATCCTCC TATACCTAGA CATCTCATCT |
| | | CAGGAAGTGG TGGTGGGGGT AGTCAGAAGG AAAAATAACT GGACATCTTT |
| | | GTGTAAACCA TAATCCACAT GTGCCGTAAA TGATCTTCAC TCCTTATCCG |
| | | AGGGCAAATT CACAAGGATC CCCAAGATCC ACTTTTAGAA GCCATTCTCA |
| | | TCCAGCAGTG AGAAGCTTCC AGGTAGGACA GAAAAAAGAT CCAGCTTCAG |
| | | CTGCACACCT CTGTCCCCTT GGATGGGGAA CTAAGGGAAA ACGTCTGTTG |
| | | TATCACTGAA GTTTTTTGTT TTGTTTTTAT ACGTGTCTGA ATAAAAATGC |
| | | CAAAGTTTTT TTTCA |
| 43 | GAP43 | ACAGTTGCTG CTAACTGCCC TGGTGTGTGT GAGGGAGAGA GAGGGAGGGA |
| | | GGGAGAGAGA GCGCGCTAGC GCGAGAGAGC GAGTGAGCAA GCGAGCAGAA |
| | | AAGAGGTGGA GAGGGGGGGA ATAAGAAAGA GAGAGAAGGA AAGGAGAGAA |
| | | GGCAGGAAGA AGGCAAGGGA CGAGACAACC ATGCTGTGCT GTATGAGAAG |
| | | AACCAAACAG AATTAAAAGG GAACCTGGTC TCTGGGTTGT TTTCAACATC |
| | | TCAAGTGTGA ATTTTCCCTG TCAAAATCTT CACAAGGAAA ATGAGTCACA |
| | | GCATCACCTG GGTGACGAGG TCATAACACC TCAGCCCTTG CTTAAAAAAT |
| | | TTTATTTCTA CTTTTCTATT GTAAAGAGAT CTCAAAACAG GAAGATAAAA |
| | | TTGGACTGAC AGCTCTACAG CCTAGTCTTT TAGACAGTGA ACTAGGCCAG |
| | | CATTGGCAGA CACTGGCGAT GACAAAGTCC TGCTCTGAAT TATGCCACCC |
| | | CGCACTCCAC TTTTTACCTT GCCTGGGAGG CTTGAGGAAA AATCTTCAGA |
| | | GAGCAGTTCG ACCTAGTCCT TATTCACTTG GCTTCTTGAC TTTCTGGATT |
| | | TCAAGGGTTG AAAAAAATGA TGACGACCAA AAGATTGAAC AAGATGGTAT |
| | | CAAACCAGAA GATAAAGCTC ATAAGGCCGC AACCAAAATT CAGGCTAGCT |
| | | TCCGTGGACA CATAACAAGG AAAAAGCTCA AAGGAGAGAA GAAGGATGAT |
| | | GTCCAAGCTG CTGAGGCTGA AGCTAATAAG AAGGATGAAG CCCCTGTTGC |
| | | CGATGGGGTG GAGAAGAAGG GAGAAGGCAC CACTACTGCC GAAGCAGCCC |
| | | CAGCCACTGG CTCCAAGCCT GATGAGCCCG GCAAAGCAGG AGAAACTCCT |
| | | TCCGAGGAGA AGAAGGGGGA GGGTGATGCT GCCACAGAGC AGGCAGCCCC |
| | | CCAGGCTCCT GCATCCTCAG AGGAGAAGGC CGGCTCAGCT GAGACAGAAA |
| | | GTGCCACTAA AGCTTCCACT GATAACTCGC CGTCCTCCAA GGCTGAAGAT |
| | | GCCCCAGCCA AGGAGGAGCC TAAACAAGCC GATGTGCCTG CTGCTGTCAC |
| | | TGCTGCTGCT GCCACCACCC CTGCCGCAGA GGATGCTGCT GCCAAGGCAA |
| | | CAGCCCAGCC TCCAACGGAG ACTGGGGAGA GCAGCCAAGC TGAAGAGAAC |
| | | ATAGAAGCTG TAGATGAAAC CAAACCTAAG GAAAGTGCCC GGCAGGACGA |
| | | GGGTAAAGAA GAGGAACCTG AGGCTGACCA AGAACATGCC TGAACTCTAA |
| | | GAAATGGCTT TCCACATCCC CACCCTCCCC TCTCCTGAGC CTGTCTCTCC |
| | | CTACCCTCTT CTCAGCTCCA CTCTGAAGTC CCTTCCTGTC CTGCTCACGT |
| | | CTGTGAGTCT GTCCTTTCCC ACCCACTAGC CCTCTTTCTC TCTGTGTGGC |
| | | AAACATTTAA AAAAAAAAAA AAAAAGCAGG AAAGATCCCA AGTCAAACAG |
| | | TGTGGCTTAA ACATTTTTTG TTTCTTGGTG TTGTTATGGC AAGTTTTTGG |
| | | TAATGATGAT TCAATCATTT TGGGAAATTC TTGCACTGTA TCCAAGTTAT |
| | | TTGATCTGGT GCGTGTGGCC CTGTGGGAGT CCACTTTCCT CTCTCTCTCT |
| | | CTCTCTGTTC CAAGTGTGTG TGCAATGTTC CGTTCATCTG AGGAGTCCAA |
| | | AATATCGAGT GAATTCAAAA TCATTTTTGT TTTCCTCCTT TTCAATGTGA |
| | | TGGAATGAAC AAAAAGGAAA AAATTCAAAA AACCCAGTTT GTTTTAAAAA |
| | | TAAATAAATA AAGCAAATGT GCCAATTAGC GTAAACTTGC GGCTCTAAGG |
| | | CTCCTTTTTC AACCCGAATA TTAATAAATC ATGAGAGTAA TCAA |
| 44 | GDNF | AGCCTGCGCT CCTGGCGCCC TCATGTCTTC ACGGGACTCC CCGCGCCGGT |
| | | TGACGTGGTG TCTCGTTCGG ATCTCCAGGC AAGACCTCAG CTCCGGCAGC |
| | | AGCATCAGAC AAACCAGTCT CGTGCTCCCA GGCAGTGCGC CCAGAGGAGG |
| | | CGCAGAGCGC GGCAGCTGCC GCTGAGCCGC CCGCAGCGCC CCGGGCCCGC |
| | | GCAGCCCCAG CCAAGAGCGC GACGCGCGCA GCCCTGTCAG CCCCCCACCC |
| | | AAAGCAGCGG CGGCTGCTCG GACCTCGGCT TCTGGGGGTG CGGGGGCCCG |
| | | GCGGGAGAGT TGCCGGCAGC CCTCGCCCTG TTGGCGGCGG CGGCGGCGGG |
| | | AGTCTTGGCC GCCGCCTCCA GCGCGCCCTT GCTGCCCCGC GCGACCCCAG |
| | | GATTGCGAAC TCTTGCCCCT GACCTGTTGG GCGGGGCTCC GCGCTCCAGC |
| | | CATCAGCCCG GATGGGTCTC CTGGCTGGGA CTTGGGGCAC CTGGAGTTAA |
| | | TGTCCAACCT AGGGTCTGCG GAGACCCGAT CCGAGGTGCC GCCGCCGGAC |
| | | GGGACTTTAA GATGAAGTTA TGGGATGTCG TGGCTGTCTG CCTGGTGCTG |
| | | CTCCACACCG CGTCCGCCTT CCCGCTGCCC GCCGGTAAGA GGCCTCCCGA |
| | | GGCGCCCGCC GAAGACCGCT CCCTCGGCCG CCGCCGCGCG CCCTTCGCGC |
| | | TGAGCAGTGA CTCAAATATG CCAGAGGATT ATCCTGATCA GTTCGATGAT |
| | | GTCATGGATT TTATTCAAGC CACCATTAAA AGACTGAAAA GGTCACCAGA |
| | | TAAACAAATG GCAGTGCTTC CTAGAAGAGA GCGGAATCGG CAGGCTGCAG |
| | | CTGCCAACCC AGAGAATTCC AGAGGAAAAG GTCGGAGAGG CCAGAGGGGC |
| | | AAAAACCGGG GTTGTGTCTT AACTGCAATA CATTTAAATG TCACTGACTT |
| | | GGGTCTGGGC TATGAAACCA AGGAGGAACT GATTTTTAGG TACTGCAGCG |
| | | GCTCTTGCGA TGCAGCTGAG ACAACGTACG ACAAAATATT GAAAAACTTA |
| | | TCCAGAAATA GAAGGCTGGT GAGTGACAAA GTAGGGCAGG CATGTTGCAG |
| | | ACCCATCGCC TTTGATGATG ACCTGTCGTT TTTAGATGAT AACCTGGTTT |
| | | ACCATATTCT AAGAAAGCAT TCCGCTAAAA GGTGTGGATG TATCTGACTC |
| | | CGGCTCCAGA GACTGCTGTG TATTGCATTC CTGCTACAGT GCAAAGAAAG |
| | | GGACCAAGGT TCCCAGGAAA TGTTTGCCCA GAATGGAAGA TGAGGACCAA |
| | | GGAGGCGGAG GAGGAGGAAG AAGAAGAGGA GGAGGAGGAG GAGGAGGAGG |
| | | AGGAGGAGGA AGGCAGCCAT CATGGGAGCC TGGTAGAGGG AGATCCAGCT |
| | | ACAGACAACT GGACAGGAGA GAGAGAAAAC AGCCCTCTGG ATTCTCCAGG |
| | | ATGGCAGCCG ATGTCACTAG AAGCTCAGGG CTGATGTTCC TGGTTGGCTA |
| | | TTGCCACCAT TTCAGCTGAT ACAGTCCACC ATCACTGATT ACCGGCGCGG |
| | | TTGCGGTGGA TGCACTTGAA CCAAACCAGT GTATCTCCTG TGATTTGTTT |
| | | TCATGTGTCC GAAGACACCA GGGAAACAGA GATCCTGGTG TTGTTCCTTG |
| | | TTATTACGTT TTACTGCTGA AAGTAAGAGG TTTATTTTTC TGTCACTCAG |
| | | TGGAGACATA CCCTGGAAAG GAGAGGGGAA AAAAAAAGCA AAGATACAAG |
| | | AGATAATCAC CTTTGCATTT GAAAGTTGAG GCCCGAGGTT TACTACAACC |
| | | AGCATTTTTG CCAACGGTTG GTGATTGATT TCCATCACGG TGTGTGGGGT |
| | | GGGAAGAAGT TGGCTAGGAA CCAAAAAGGC TGTGCTCATG ATTAAACACA |
| | | AACCTGAAGG TATTTCCTTT ATGTCCTTGG AAACAGGAAA CGAGTTGTGG |
| | | TTTTCGCCAG CATTCTTGTA GGAGAGAATC GGGGAAGGCC CCGAACTGCC |
| | | CCCGGGCAGG GAGAGCCCCT CAGGCCTGTT GGTTTACAGA GAGACAGATG |
| | | TTACATAACC AGCTCCGTTG ATGCGTGGTC ACCAGTGACC AGAGAAGCTA |
| | | CTCGATGCAA TGCATCTGTT TCAGATACAG AAATATAGAG AAGATATTTA |
| | | TTGAAATTTA AGTTATTGTT ATTTATTACC GTTCACTAAT GAATTTCTCT |
| | | TTTTTCCCTT ATTTATTAAA GTTTCTTTTC AAAGGTGCCA AAGTATATGT |
| | | GCTCGCAAAA TGCAAAGAAA GGTGACAAAA GGAAATTTGA ATTGGGAACA |
| | | AGGGTCCATG CTTTTCAAAG TATTAAAAAG TTTTTTGCCA GGCAAAAATC |
| | | ACTTACTTTA CCTTTTTAAG AAAATTTGTC ATTAATTTTC CCCAGATTTC |
| | | AGCATTTTTC CCAATTTTTA TTTGTGGAGC ATCTCAGGCA AGCCCCCTTT |
| | | CCTGGAGCAG CGTGCAGAGA CCACTGGCAC TTGACTTTAT TTCTTCCTTG |
| | | CTCCATTGCT GAACAGAAAT GTCGTGGGCT CCACTTCCTG TTGTCTTTAA |
| | | GCTCTTAGTC CCCTCCACGT ATACCTATCT GTACTATGCA TAACCATATG |
| | | TAGAAAAGGT TCAGTTCCTT TTAGTAGGTA GTCCTGGATT TAATGCTGAC |
| | | CTAAAAGTAA TGTCGACAAT GCTGTCAGGT AGCTGCCGTT CTACCGACTC |
| | | CCTCCATCCC TGCCCACCCA CTGCCCTCCC GAGAATATGC TGGCTGCCCA |
| | | GTGCAGCCCG GGAGACACAG GGGCCTTCCA GAGGTAGGGT CTACCAGGTC |
| | | CTGTACAACC CCTGGGCTGT CACCGGGGGT CAACAGCTGC TGCTCCTATA |
| | | TACCCAAACA CCTGACAGCT CCCTGGGGAG CAGATGGCTG AGAAGGGTGC |
| | | TGAGGAAGCC ATATTGGGAC CAGCCACAGC CACACACATG GAGCCTCATA |
| | | CTTAGGAGCG TGCTGCCTTT AAATGAAGGT GGTCGGGGCC AGTGCAGCGG |
| | | CTCACACCCA TAATCCCAAC ACTTTGGAAA GCCAAGGTGG GAGGATCTCT |
| | | TGAACCCAGG AGTTTGAGAC CAGCTTGGGC AACATAGGGA GACCCTGTCT |
| | | CTACAGAAAC TTTAAAAATT AGGCAGGCAT GATGGTGCAC ACCTGTGGTC |
| | | CCAGCTACTC AAGAGGCTGA AGGAGGATCA CTTGAGTCCA GAAGGTCGAG |
| | | GCTGCAGTGA GCTGTGATCA TGCCACTGCA CTCCAGCCTA AGTGACAGTG |
| | | CGGTACCCTG TCTCAAAAAA AAAAAAAAAA AAAAAAAGAG GTTGGAGCAG |
| | | GAGGAAGCAT AGGGGCGGGA ACAGCCACCT CCTCCATGCC CTAGATTGTG |
| | | AATTTATCGG GCAGCCAACA CATGTATGAC ACACTAGGCC CTGTATTACA |
| | | GCTTGTTACG CATTTCATAA AAGGGATTTT CATTAAGGAG ATAATCTATT |
| | | ACTACCTACC TTAGTGGCTA CTAGTATAAA ACTATGACAG ATTTAGCAAT |
| | | TAAATGAAAT ACTGGCCTCC ATCAAATAAT CATAGTAACA AGAAGCAGCA |
| | | GTTACCAGAC ATCTGATCCC CTTCCCCCAA AATACCCAAA TTCTTCATGG |
| | | TTCTGCCCTT CTCTGTCCTT TCTGCTCCCC TTGCTCGCCT GGGAAATGGA |
| | | GGAAAGGCCT TCCCTCTCAC ACTGTCTTGG GATCTTGCTG AGAATTCAGA |
| | | CTGCTCGAAA CAGTGACAAA CCCCAGCCAT CCAGTCATTC GTGGAGCACA |
| | | ATTTGGATGT GGCCCCAGGG GCATCTGTCC CATTCAGAGA ACCTTGGCAG |
| | | TGCGATGGCC ACTGTTCCCA GGCTTCAACC TCAGTGACCC CCCCCAACAA |
| | | CTCCCCATGG AGAGTCCCTG CCCAAAAAAG CTGTAGGATC CAAGGGGTGT |
| | | CAATAGCTCG TTCCCGGCAT CACCTACACA CCACAAGCAG GTTTTAATGG |
| | | AAGCAAGTTG CTCCACCAAA TCCACAAAAG GGTAAAGTTT GTGATTTTTC |
| | | TTTATCATTG CGATCACCAT CTGATACCGT AAGGAGTGCA CTTGTTTGGA |
| | | AGTTCTGACT TCTCTGATCT GTCTTGGTCG TTTGTGTTAT AAAACCAAAG |
| | | TTCTCTACAG ACTTTATTTT TGTACAATAT CATTTTGTAA CTTTTTACAA |
| | | ATAAAAACTC ATTTCTATTG C |
| 45 | MAG | GAGCTGGGCT GGCGGAGCAG AGGTGCAGAA GCAACTGAGT CCAAGTTGTC |
| | | TGGCGGCTTC AGGTGGACCC AGAAGACGTC CCCAACTCAG GGAGATTCAG |
| | | CGATCACTCA CTCGCTGTAC AGAATGATAT TCCTCACGGC ACTGCCTCTG |
| | | TTCTGGATTA TGATTTCAGC CTCCCGAGGG GGTCACTGGG GTGCCTGGAT |
| | | GCCCTCGTCC ATCTCGGCCT TCGAAGGCAC GTGCGTCTCC ATCCCCTGCC |
| | | GCTTTGACTT CCCGGATGAG CTGCGGCCCG CTGTGGTGCA TGGTGTCTGG |
| | | TACTTCAATA GCCCCTACCC CAAGAACTAC CCCCCGGTGG TCTTCAAGTC |
| | | GCGCACCCAA GTAGTCCACG AGAGCTTCCA GGGCCGCAGC CGCCTCCTGG |
| | | GGGACCTGGG CCTGCGAAAC TGCACCCTCC TGCTCAGCAA CGTCAGCCCC |
| | | GAGCTGGGCG GGAAGTACTA CTTCCGTGGG GACCTGGGCG GCTACAACCA |
| | | GTACACCTTC TCAGAGCACA GCGTCCTGGA TATCGTCAAC ACCCCCAACA |
| | | TCGTGGTGCC CCCAGAGGTG GTGGCAGGCA CGGAGGTGGA GGTCAGCTGC |
| | | ATGGTGCCGG ACAACTGCCC AGAGCTGCGC CCTGAGCTGA GCTGGCTGGG |
| | | CCACGAGGGG CTGGGGGAGC CCGCTGTGCT GGGCCGGCTG CGGGAGGACG |
| | | AGGGCACCTG GGTGCAGGTG TCACTGCTGC ACTTCGTGCC CACGAGGGAG |
| | | GCCAACGGCC ACAGGCTGGG CTGCCAGGCC TCCTTCCCCA ACACCACCCT |
| | | GCAGTTCGAG GGCTACGCCA GCATGGACGT CAAGTACCCC CCGGTGATTG |
| | | TGGAGATGAA CTCCTCGGTG GAGGCCATCG AGGGCTCCCA CGTGAGCCTG |
| | | CTCTGTGGGG CTGACAGCAA CCCCCCGCCG CTGCTGACCT GGATGCGGGA |
| | | CGGGACAGTC CTCCGGGAGG CGGTGGCCGA GAGCCTGCTC CTGGAGCTGG |
| | | AGGAGGTGAC CCCCGCCGAA GACGGCGTCT ATGCCTGCCT GGCCGAGAAT |
| | | GCCTATGGCC AGGACAACCG CACCGTGGGG CTCAGTGTCA TGTATGCACC |
| | | CTGGAAGCCA ACAGTGAACG GGACAATGGT GGCCGTAGAG GGGGAGACGG |
| | | TCTCTATCTT GTGCTCCACA CAGAGCAACC CGGACCCTAT TCTCACCATC |
| | | TTCAAGGAGA AGCAGATCCT GTCCACGGTC ATCTACGAGA GCGAGCTGCA |
| | | GCTGGAGCTG CCGGCCGTGT CACCCGAGGA TGATGGAGAG TACTGGTGTG |
| | | TGGCTGAGAA CCAGTATGGC CAGAGGGCCA CCGCCTTCAA CCTGTCTGTG |
| | | GAGTTCGCCC CTGTGCTCCT CCTGGAGTCC CACTGCGCGG CAGCCCGAGA |
| | | CACGGTGCAG TGCCTGTGCG TGGTGAAGTC CAACCCGGAG CCGTCCGTGG |
| | | CCTTTGAGCT GCCATCGCGC AATGTGACCG TGAACGAGAG CGAGCGGGAG |
| | | TTCGTGTACT CGGAGCGCAG CGGCCTCGTG CTCACCAGCA TCCTCACGCT |
| | | GCGGGGGCAG GCCCAGGCCC CGCCCCGCGT CATCTGCACC GCGAGGAACC |
| | | TCTATGGCGC CAAGAGCCTG GAGCTGCCCT TCCAGGGAGC CCATCGACTG |
| | | ATGTGGGCCA AGATCGGGCC TGTGGGCGCC GTGGTCGCCT TTGCCATCCT |
| | | GATTGCCATC GTCTGCTACA TTACCCAGAC ACGCAGGAAA AAGAACGTGA |
| | | CAGAGAGCCC CAGCTTCTCG GCAGGGGACA ACCCTCCCGT CCTGTTCAGC |
| | | AGCGACTTCC GCATCTCTGG GGCACCAGAG AAGTACGAGT CCAAAGAGGT |
| | | TTCTACCCTG GAATCTCACT GAGTGCCCCA GGAGAGCGAG AGGCGCCTGG |
| | | GATCTGAGAG GAGGCTGCTG GGCCTTCGGG GTGAGCCCCC AGAGCTGGAC |
| | | CTGAGCTATT CTCACTCGGA CCTGGGGAAA CGGCCCACCA AGGACAGCTA |
| | | CACGCTGACG GAGGAGCTAG CTGAGTATGC TGAAATCCGG GTCAAGTGAA |
| | | GGAGCTGGGG GCAGCCTGCG TGGCTGACCC CCCTCAGGAC CCTCGCTGGC |
| | | CCCCACTGGC TGTGGGCTCC CTTCCTCCCA AAAGTATCGG GGGCTGGGGC |
| | | AGGAGGGGAG TGAGGCAGGT GACAGTGAGG TCCTGGGGGC CTGACCTCCC |
| | | CCTCCTTCCC AGCTGCCCCT CCCTGCCAGC ACCCCCACGC CCTCATTACG |
| | | GCTCCTCTCT AACCTCCTTT ACCCTCATCT GTCTGGAGGG GAGCTCTGTC |
| | | TGTCCGTGTT ATTTATTGCT ACTTCCTGCC TGGTCTCCTG CCCCCACACC |
| | | TGGCCCTGGG GCCTGTACAA AAGGGACATG AAATAAATGC CCCAAAGCCA |
| | | AA |
| 46 | MPZ | AGTTCCTGGT CCCCCACTTT CTCAACCCCA CAGATGCTCC GGGCCCCTGC |
| | | CCCTGCCCCA GCTATGGCTC CTGGGGCTCC CTCATCCAGC CCCAGCCCTA |
| | | TCCTGGCTGT GCTGCTCTTC TCTTCTTTGG TGCTGTCCCC GGCCCAGGCC |
| | | ATCGTGGTTT ACACCGACAG GGAGGTCCAT GGTGCTGTGG GCTCCCGGGT |
| | | GACCCTGCAC TGCTCCTTCT GGTCCAGTGA GTGGGTCTCA GATGACATCT |
| | | CCTTCACCTG GCGCTACCAG CCCGAAGGGG GCAGAGATGC CATTTCGATC |
| | | TTCCACTATG CCAAGGGACA ACCCTACATT GACGAGGTGG GGACCTTCAA |
| | | AGAGCGCATC CAGTGGGTAG GGGACCCTCG CTGGAAGGAT GGCTCCATTG |
| | | TCATACACAA CCTAGACTAC AGTGACAATG GCACGTTCAC TTGTGACGTC |
| | | AAAAACCCTC CAGACATAGT GGGCAAGACC TCTCAGGTCA CGCTGTATGT |
| | | CTTTGAAAAA GTGCCAACTA GGTACGGGGT CGTTCTGGGA GCTGTGATCG |
| | | GGGGTGTCCT CGGGGTGGTG CTGTTGCTGC TGCTGCTTTT CTACGTGGTT |
| | | CGGTACTGCT GGCTACGCAG GCAGGCGGCC CTGCAGAGGA GGCTCAGTGC |
| | | TATGGAGAAG GGGAAATTGC ACAAGCCAGG AAAGGACGCG TCGAAGCGCG |
| | | GGCGGCAGAC GCCAGTGCTG TATGCAATGC TGGACCACAG CAGAAGCACC |
| | | AAAGCTGTCA GTGAGAAGAA GGCCAAGGGG CTGGGGGAGT CTCGCAAGGA |
| | | TAAGAAATAG CGGTTAGCGG GCCGGGCGGG GGATCGGGGG TTAGGGGTGG |
| | | AGTCCGCCAA AGGCCCAAAG GTGATGGTCA TCGAGATGGA GCTACGAAAG |
| | | GATGAGCAGA GCCCGGAGCT CCGGCCTGCT GTCAAGTCCC CCAGCAGAAC |
| | | CAGCCTCAAA AACGCCCTCA AGAACATGAT GGGCCTGAAC TCGGACAAGT |
| | | GATCGCCACC CCCCCACCCC AGGCCCTGCC AGAGCAGGGG GACCTAGGCT |
| | | CCTCTTACCC CCGTCTAGGT GCTTTCCCTC TTTGCTCCCC CGCCCTGCCC |
| | | TGCCCTCACC TCCCTTTGAG ATGTAAGTTT CATTCCAGAA TTCATTCCCC |
| | | AGGCAATTGT ATTCTCCCCC ACCTTCACCC CTGGCTTTCT GGGAGCCCAG |
| | | GAGCTAATCC TACCCCTCAC CTGCCCCGGG GGCTGTGTGT TTGGTGCCTG |
| | | TCCACCTGAG CACTGAGAAG AAAGGGACTT TGATACCCTC TGCCTCAAGT |
| | | CCAGGCCACC TGGCATTCCC ATCTCCTGCA TCCCCCAGCC TGTCCCCCTG |
| | | GCTGTTTCCT CTCCCGTCCC TCCCTCCCCT CTACCAGGTG GCCCAGCTCC |
| | | ATACTCTGTC CCCCCAGCTA ATACCCAGAG CACCCAGATC AGACTCTCCT |
| | | TCAGGGTTTA TTTAGGTTAT TATTTTTTAT TTTTTAATCC ATTCTTTGTT |
| | | TGTTTACCTG TGCTCATCCT CTGCCCTTAC ACCCATGACT GAGGACCAAT |
| | | GACGTCATGT GGCTTTTGCA ATTCACGCCC CCCTTAAGTC CTTAATGAAG |
| | | AGCCAGCCCA AGTAGAGGGG CCCCTGATCC TCACACTTCA GTATAGCATT |
| | | GGTTCCCCCT GACCACTTTG GAGCACTGTT CTGGGACTCC AGGTCTTGAG |
| | | GAGAGAGACA GAGAGAGAGA ATGGATCCTC ATAGGTCAGG GAGTGGGGGA |
| | | GGGGGCAAAT GAGCCTTAAG AAATGGTTTT TAAACAACCA AACAAAAAGC |
| | | AGGAAAAACA AATGGGAAAT GGGGGGGCGG GGGGGAGGAA GAGGCTGCAC |
| | | TGCAGCCACA GGGGATTCTT AGGATTTTTC TACATTCTGT ATATTTCTTC |
| | | TCAAACCTCC AAATGTCCTT AAATGTTTAA TAAACACTGA CATTTCCAGA |
| | | A |
| 47 | NGFR | AGAGCGAGCC GAGCCGCGGC CAGCTCCGGC GGGCAGGGGG GGCGCTGGAG |
| | | CGCAGCGCAG CGCAGCCCCA TCAGTCCGCA AAGCGGACCG AGCTGGAAGT |
| | | CGAGCGCTGC CGCGGGAGGC GGGCGATGGG GGCAGGTGCC ACCGGCCGCG |
| | | CCATGGACGG GCCGCGCCTG CTGCTGTTGC TGCTTCTGGG GGTGTCCCTT |
| | | GGAGGTGCCA AGGAGGCATG CCCCACAGGC CTGTACACAC ACAGCGGTGA |
| | | GTGCTGCAAA GCCTGCAACC TGGGCGAGGG TGTGGCCCAG CCTTGTGGAG |
| | | CCAACCAGAC CGTGTGTGAG CCCTGCCTGG ACAGCGTGAC GTTCTCCGAC |
| | | GTGGTGAGCG CGACCGAGCC GTGCAAGCCG TGCACCGAGT GCGTGGGGCT |
| | | CCAGAGCATG TCGGCGCCGT GCGTGGAGGC CGACGACGCC GTGTGCCGCT |
| | | GCGCCTACGG CTACTACCAG GATGAGACGA CTGGGCGCTG CGAGGCGTGC |
| | | CGCGTGTGCG AGGCGGGCTC GGGCCTCGTG TTCTCCTGCC AGGACAAGCA |
| | | GAACACCGTG TGCGAGGAGT GCCCCGACGG CACGTATTCC GACGAGGCCA |
| | | ACCACGTGGA CCCGTGCCTG CCCTGCACCG TGTGCGAGGA CACCGAGCGC |
| | | CAGCTCCGCG AGTGCACACG CTGGGCCGAC GCCGAGTGCG AGGAGATCCC |
| | | TGGCCGTTGG ATTACACGGT CCACACCCCC AGAGGGCTCG GACAGCACAG |
| | | CCCCCAGCAC CCAGGAGCCT GAGGCACCTC CAGAACAAGA CCTCATAGCC |
| | | AGCACGGTGG CAGGTGTGGT GACCACAGTG ATGGGCAGCT CCCAGCCCGT |
| | | GGTGACCCGA GGCACCACCG ACAACCTCAT CCCTGTCTAT TGCTCCATCC |
| | | TGGCTGCTGT GGTTGTGGGC CTTGTGGCCT ACATAGCCTT CAAGAGGTGG |
| | | AACAGCTGCA AGCAGAACAA GCAAGGAGCC AACAGCCGGC CAGTGAACCA |
| | | GACGCCCCCA CCAGAGGGAG AAAAACTCCA CAGCGACAGT GGCATCTCCG |
| | | TGGACAGCCA GAGCCTGCAT GACCAGCAGC CCCACACGCA GACAGCCTCG |
| | | GGCCAGGCCC TCAAGGGTGA CGGAGGCCTC TACAGCAGCC TGCCCCCAGC |
| | | CAAGCGGGAG GAGGTGGAGA AGCTTCTCAA CGGCTCTGCG GGGGACACCT |
| | | GGCGGCACCT GGCGGGCGAG CTGGGCTACC AGCCCGAGCA CATAGACTCC |
| | | TTTACCCATG AGGCCTGCCC CGTTCGCGCC CTGCTTGCAA GCTGGGCCAC |
| | | CCAGGACAGC GCCACACTGG ACGCCCTCCT GGCCGCCCTG CGCCGCATCC |
| | | AGCGAGCCGA CCTCGTGGAG AGTCTGTGCA GTGAGTCCAC TGCCACATCC |
| | | CCGGTGTGAG CCCAACCGGG GAGCCCCCGC CCCGCCCCAC ATTCCGACAA |
| | | CCGATGCTCC AGCCAACCCC TGTGGAGCCC GCACCCCCAC CCTTTGGGGG |
| | | GGGCCCGCCT GGCAGAACTG AGCTCCTCTG GGCAGGACCT CAGAGTCCAG |
| | | GCCCCAAAAC CACAGCCCTG TCAGTGCAGC CCGTGTGGCC CCTTCACTTC |
| | | TGACCACACT TCCTGTCCAG AGAGAGAAGT GCCCCTGCTG CCTCCCCAAC |
| | | CCTGCCCCTG CCCCGTCACC ATCTCAGGCC ACCTGCCCCC TTCTCCCACA |
| | | CTGCTAGGTG GGCCAGCCCC TCCCACCACA GCAGGTGTCA TATATGGGGG |
| | | GCCAACACCA GGGATGGTAC TAGGGGGAAG TGACAAGGCC CCAGAGACTC |
| | | AGAGGGAGGA ATCGAGGAAC CAGAGCCATG GACTCTACAC TGTGAACTTG |
| | | GGGAACAAGG GTGGCATCCC AGTGGCCTCA ACCCTCCCTC AGCCCCTCTT |
| | | GCCCCCCACC CCAGCCTAAG ATGAAGAGGA TCGGAGGCTT GTCAGAGCTG |
| | | GGAGGGGTTT TCGAAGCTCA GCCCACCCCC CTCATTTTGG ATATAGGTCA |
| | | GTGAGGCCCA GGGAGAGGCC ATGATTCGCC CAAAGCCAGA CAGCAACGGG |
| | | GAGGCCAAGT GCAGGCTGGC ACCGCCTTCT CTAAATGAGG GGCCTCAGGT |
| | | TTGCCTGAGG GCGAGGGGAG GGTGGCAGGT GACCTTCTGG GAAATGGCTT |
| | | GAAGCCAAGT CAGCTTTGCC TTCCACGCTG TCTCCAGACC CCCACCCCTT |
| | | CCCCACTGCC TGCCCACCCG TGGAGATGGG ATGCTTGCCT AGGGCCTGGT |
| | | CCATGATGGA GTCAGGTTTG GGGTTCGTGG AAAGGGTGCT GCTTCCCTCT |
| | | GCCTGTCCCT CTCAGGCATG CCTGTGTGAC ATCAGTGGCA TGGCTCCAGT |
| | | CTGCTGCCCT CCATCCCGAC ATGGACCCGG CTCACCTTGC AACACACAGA |
| | | CACACGCACA CACACACACA GGAGGAGAAA AGCTAACACT GGCCCCTAGA |
| | | ATCAGCCTAG GGGTCAGGGA CCAAGGACCC CTCACCTTGC AACACACAGA |
| | | CACACGCACA CACACACACA GGAGGAGAAA TCTCACTTTT CTCCATGAGT |
| | | TTTTTCTCTT GGGCTGAGAC TGGATACTGC CCGGGGCAGC TGCCAGAGAA |
| | | GCATCGGAGG GAATTGAGGT CTGCTCGGCC GTCTTCACTC GCCCCCGGGT |
| | | TTGGCGGGCC AAGGACTGCC GACCGAGGCT GGAGCTGGCG TCTGTCTTCA |
| | | AGGGCTTACA CGTGGAGGAA TGCTCCCCCA TCCTCCCCTT CCCTGCAAAC |
| | | ATGGGGTTGG CTGGGCCCAG AAGGTTGTGA TGAAGAAAAG TGGGCCAGTG |
| | | TGGGAATGCG GCAAGAAGGA ATTGACTTCG ACTGTGACCT GTGGGGATTT |
| | | CTCCCAGCTC TAGACAACCC TGCAAAGGAC TGTTTTTTCC TGAGCTTGGC |
| | | CAGAAGGGGG CCATGAGGCC TCAGTGGACT TTCCACCCCC TCCCTGGCCT |
| | | GTTCTGTTTT GCCTGAAGTT GGAGTGAGTG TGGCTCCCCT CTATTTAGCA |
| | | TGACAAGCCC CAGGCAGGCT GTGCGCTGAC AACCACCGCT CCCCAGCCCA |
| | | GGGTTCCCCC AGCCCTGTGG AAGGGACTAG GAGCACTGTA GTAAATGGCA |
| | | ATTCTTTGAC CTCAACCTGT GATGAGGGGA GGAAACTCAC CTGCTGGCCC |
| | | CTCACCTGGG CACCTGGGGA GTGGGACAGA GTCTGGGTGT ATTTATTTTC |
| | | CTCCCCAGCA GGTGGGGAGG GGGTTTGGGG GCTTGCAAGT ATGTTTTAGC |
| | | ATGTGTTTGG TTCTGGGGCC CCTTTTTACT CCCCTTGAGC TGAGATGGAA |
| | | CCCTTTTGGC CCCCGAGCTG GGGGCCATGA GCTCCAGACC CCCAGCAACC |
| | | CTCCTATCAC CTCCCCTCCT TGCCTCCTGT GTAATCATTT CTTGGGCCCT |
| | | CCTGAAACTT ACACACAAAA CGTTAAGTGA TGAACATTAA ATAGCAAAGA |
| | | AAGAAAAA |
| 48 | PLLP | GACGCCTCCT GCAGCGCCTG GAGCCACACA GGGATCCGGA GCCTGGGGGA |
| | | AAAGCGGCGC GGGAGCCGGC ACCCACCGCT GGAGGGGCGG CGACGGCGGC |
| | | CGTAGCGACC TCGGGAGGCA AGCGGAGCCG CCATGGCCGA GTTCCCGTCG |
| | | AAAGTTAGCA CGCGGACCAG CAGTCCTGCG CAGGGCGCCG AAGCCTCGGT |
| | | GTCGGCGCTG CGCCCGGACC TGGGCTTCGT GCGCTCCCGC CTCGGGGCGC |
| | | TCATGCTGCT GCAGCTGGTG CTGGGGCTGC TGGTGTGGGC GCTGATTGCG |
| | | GACACCCCGT ACCACCTGTA TCCGGCCTAT GGCTGGGTGA TGTTCGTCGC |
| | | TGTCTTCCTC TGGCTGGTGA CAATCGTCCT CTTCAACCTC TACCTGTTTC |
| | | AGCTGCACAT GAAGTTGTAC ATGGTTCCCT GGCCACTGGT GTTAATGATC |
| | | TTTAACATCA GCGCCACCGT TCTCTACATC ACCGCCTTCA TCGCCTGCTC |
| | | TGCGGCAGTT GACCTGACAT CCCTGAGGGG CACCCGGCCT TATAACCAGC |
| | | GCGCGGCTGC CTCGTTCTTT GCGTGTTTGG TGATGATCGC CTATGGAGTG |
| | | AGTGCCTTCT TCAGCTACCA GGCCTGGCGA GGAGTAGGCA GCAATGCGGC |
| | | CACCAGTCAG ATGGCTGGCG GCTATGCCTA AACCACCTGT GCCACGGCCC |
| | | CCTCTGGGGC TGAAGCCGCC GCTGGGTCAC AGAGCAGGGT CACCCTGCAA |
| | | GCCTGAAGCT GGGGAGCCCT GCGTGGAGTC AGCCCAACAG GGACTGCATT |
| | | TGCTCCTCTC TGCCCGTCAG ACATAAGCTC TCACAGCGCT AAGGAAGCAG |
| | | GCCCAGGCTG GCAGGCATCT CGGCTTGCAG GAGGCCAACT GCTGAGACCT |
| | | CTTCTCCATC CCCCTTATTC AGTGGAAGAT GACGGGGGAT CTGAGGCTGT |
| | | GTCTCTGCCT TGTCTTTAGA GGACTTCAGC GTCCAAGACT GGGGCCCACC |
| | | CTTCTCACCA GCACTAAATG CACTAACAAG GACTCCAGAC CTGCAGCCCC |
| | | AGACCCGCCG TAGTATAAGC CTAACAAGCA ACACGTAGCA CCTTAGTCTT |
| | | TGTTCCAGGA GAGCTGAGCA AGCTGGTGAA ACCACTCTCC TTCCTTTAAA |
| | | CACCGTTTCA ACCAACCTCT CCCTGGAGCC AACCTGTAAA AAGTGGGTTG |
| | | ATTGCTGACA GCATGGTCTT CCCTCCCTGC ATTTCAGACA TACCAGTTAC |
| | | TGAAAGCAAA TCAGTTTTAA GTGATTTCTC AGTGCTGAAA AGCCTGTCCA |
| | | GGTTTCCTTC CCTTTCCCAA GCCTCTCTCT GTAATACTCC CTTTGGGCGA |
| | | AGCTAACATC GGTGCCTCCC CGACCTTGCT GACTAGGCAC ATGGGACGCA |
| | | AAGGAGGGAG GGAAGCAAGG CCTTGCCTGG CGAGTTGTCA TGTGGTTGGT |
| | | GGTGACTGTT TTATTTTTTT TAATAAAAAT AAAGATGAGA GAAATTA |
| 49 | PLXNB3 | GAGACGTGCT CCTGGCACCG CCAGCTGCTA CTTGGCCCTC GCCGGTGGCC |
| | | CACCAGGACA ATGCCCCCCC GCAGCCATCT CATGCCCATC GCCACTGCCC |
| | | TGGGGCAGCT GAACTGAGCG TATGTGCCAC GCCGCCCAGG AGACCCCTCT |
| | | GCTGCACCAC TTCATGCACC CCCGCTGCCT TGCCCCAGTG CTTCCTCGCC |
| | | CTGGAGCTGG GCGCCAGCAT GGAGCTCACC CCTGCCTCTT CGCTGACTTG |
| | | CTCCTTGCTC AGCCCGCGGC TGCCTGGCTC TTTCCCCCAG CTGCGGAGGG |
| | | TTCCTCCTTG CAGCCGGCCC TGGCTGCCCA AGGCCCCCGT GATGGCTCGC |
| | | TGGCCTCCCT TCGGCCTCTG CCTCCTCCTG CTGCTGCTGT CCCCACCGCC |
| | | ACTGCCCTTG ACAGGGGCCC ATCGCTTCTC CGCACCTAAT ACCACTCTCA |
| | | ACCACTTGGC ACTGGCACCT GGCCGAGGCA CACTCTATGT CGGCGCAGTG |
| | | AACCGCCTCT TCCAGCTCAG CCCCGAGCTG CAGCTCGAGG CCGTGGCTGT |
| | | CACTGGCCCT GTAATCGACA GCCCTGACTG CGTGCCCTTC CGTGACCCAG |
| | | CCGAGTGCCC ACAGGCCCAG CTCACTGACA ATGCCAACCA GCTGCTGCTG |
| | | GTGAGCAGCC GCGCCCAGGA GCTGGTGGCC TGCGGGCAGG TGCGGCAGGG |
| | | CGTGTGTGAG ACACGGCGCC TTGGGGATGT GGCCGAGGTG CTGTACCAGG |
| | | CTGAGGACCC TGGTGACGGG CAGTTTGTGG CTGCCAATAC CCCGGGAGTG |
| | | GCAACGGTGG GGCTGGTGGT GCCCTTGCCC GGCCGGGACC TCCTGCTTGT |
| | | GGCCAGAGGC CTGGCGGGCA AGCTGTCGGC AGGGGTGCCA CCCCTGGCCA |
| | | TCCGCCAGCT GGCCGGGTCT CAGCCCTTCT CCAGCGAGGG CCTGGGCCGC |
| | | CTGGTGGTGG GCGACTTCTC CGACTACAAC AACAGCTACG TCGGGGCCTT |
| | | TGCCGACGCC CGCTCCGCCT ACTTCGTGTT CCGCCGCCGC GGGGCCCGGG |
| | | CCCAGGCTGA GTACCGCTCC TACGTGGCCC GCGTCTGCCT GGGGGACACC |
| | | AACCTGTACT CCTACGTGGA GGTCCCCCTC GCCTGCCAGG GCCAGGGCCT |
| | | CATCCAGGCC GCCTTCCTTG CCCCGGGCAC CTTGCTAGGG GTGTTTGCCG |
| | | CGGGCCCAAG GGGCACCCAG GCGGCGCTCT GTGCCTTCCC CATGGTGGAG |
| | | CTGGGTGCCA GCATGGAGCA GGCCCGGAGA CTCTGCTACA CGGCGGGCGG |
| | | CCGGGGCCCC AGCGGCGCAG AGGAAGCCAC CGTGGAGTAC GGCGTCACGT |
| | | CGCGCTGCGT CACCCTGCCC CTTGATTCCC CCGAGTCGTA CCCCTGTGGC |
| | | GACGAGCACA CCCCCAGCCC CATTGCTGGC CGCCAGCCCC TGGAGGTCCA |
| | | GCCTCTGCTG AAGCTCGGGC AGCCGGTCAG CGCCGTGGCA GCTCTCCAGG |
| | | CAGATGGGCA CATGATAGCC TTCCTGGGGG ACACCCAGGG CCAGCTGTAC |
| | | AAGGTCTTTC TCCACGGCTC CCAGGGCCAG GTTTACCACT CCCAGCAAGT |
| | | GGGGCCTCCA GGCTCAGCCA TCAGCCCAGA CCTGCTGCTG GACAGCAGTG |
| | | GCAGTCACCT CTATGTCCTG ACTGCCCACC AGGTGGACCG GATACCTGTG |
| | | GCAGCCTGCC CCCAGTTCCC TGACTGTGCC AGCTGCCTCC AGGCCCAGGA |
| | | CCCGCTGTGT GGCTGGTGTG TCCTCCAGGG CAGGTGTACC CGGAAGGGCC |
| | | AGTGCGGGCG GGCAGGCCAG CTGAACCAGT GGCTGTGGAG TTATGAGGAG |
| | | GACAGCCACT GCCTGCACAT CCAGAGCCTG CTGCCGGGCC ACCACCCCCG |
| | | CCAGGAGCAG GGCCAGGTCA CTTTGTCTGT CCCCCGGCTG CCCATCCTGG |
| | | ATGCAGATGA ATACTTCCAT TGTGCGTTCG GGGACTATGA CAGCTTGGCT |
| | | CATGTGGAAG GGCCCCACGT GGCCTGTGTC ACCCCTCCCC AAGACCAGGT |
| | | GCCACTTAAC CCTCCAGGCA CAGACCACGT CACTGTGCCC CTGGCCCTGA |
| | | TGTTCGAGGA CGTGACTGTG GCTGCCACCA ACTTCTCCTT TTATGACTGC |
| | | AGTGCCGTCC AGGCCTTGGA GGCGGCTGCC CCGTGTCGCG CTTGCGTGGG |
| | | CAGCATCTGG CGGTGTCACT GGTGCCCGCA GAGTAGCCAC TGCGTGTACG |
| | | GAGAGCACTG CCCAGAGGGC GAGAGGACCA TCTACAGCGC CCAGGAGGTG |
| | | GACATCCAGG TGCGTGGCCC AGGGGCTTGC CCACAGGTCG AAGGCCTGGC |
| | | AGGTCCCCAC CTGGTGCCTG TGGGCTGGGA GAGCCATTTG GCCCTACGCG |
| | | TGCGGAACCT TCAACATTTC CGAGGCCTGC CTGCCTCCTT CCACTGCTGG |
| | | CTGGAGCTGC CTGGAGAACT TCGGGGACTG CCGGCCACCC TGGAGGAGAC |
| | | AGCAGGGGAT TCAGGCCTCA TCCACTGCCA GGCCCACCAG TTTTATCCCT |
| | | CCATGTCCCA GCGGGAGCTC CCAGTGCCCA TCTACGTCAC CCAGGGTGAA |
| | | GCCCAGAGGC TGGACAACAC CCATGCTCTT TATGTGATCC TGTACGACTG |
| | | CGCCATGGGC CACCCGGACT GCAGCCACTG CCAAGCGGCC AACAGGAGCC |
| | | TGGGCTGCCT GTGGTGTGCT GACGGCCAGC CTGCCTGTCG CTATGGGCCC |
| | | TTGTGCCCGC CGGGGGCTGT GGAGCTGCTG TGTCCTGCGC CCAGCATTGA |
| | | TGCAGTCGAG CCCCTGACCG GTCCCCCTGA GGGAGGCTTG GCCCTCACCA |
| | | TCCTGGGCTC CAACCTGGGC CGGGCCTTCG CCGATGTGCA GTACGCCGTG |
| | | AGCGTGGCCA GCCGGCCCTG CAACCCTGAG CCCTCTCTCT ACCGCACGTC |
| | | GGCCCGGATT GTGTGTGTGA CATCTCCTGC CCCCAATGGC ACCACTGGGC |
| | | CCGTCCGGGT GGCCATTAAG AGCCAGCCAC CAGGCATCTC AAGCCAGCAC |
| | | TTCACCTACC AGGACCCTGT CCTGCTGAGC CTGAGTCCTC GCTGGGGCCC |
| | | CCAGGCAGGG GGCACCCAGC TCACCATCCG AGGTCAGCAC CTCCAGACAG |
| | | GTGGCAACAC CAGTGCCTTC GTGGGTGGCC AACCCTGTCC CATCCTGGAG |
| | | CCAGTGTGTC CGGAGGCCAT CGTGTGCCGT ACCAGGCCCC AGGCTGCCCC |
| | | AGGAGAAGCA GCGGTCCTTG TGGTCTTTGG CCATGCCCAG CGCACACTGC |
| | | TCGCCAGCCC CTTCCGCTAC ACCGCCAACC CCCAGCTTGT AGCGGCGGAG |
| | | CCCAGTGCCA GCTTCCGGGG GGGTGGGCGA CTGATCCGTG TCAGGGGCAC |
| | | CGGCCTAGAC GTGGTGCAGC GGCCCCTACT GTCTGTGTGG CTGGAGGCTG |
| | | ACGCAGAGGT GCAGGCTTCC AGGGCCCAGC CCCAGGACCC ACAGCCAAGG |
| | | AGGAGCTGTG GAGCCCCTGC TGCGGACCCC CAGGCTTGTA TCCAGCTCGG |
| | | TGGGGGGCTG CTGCAGTGCT CCACCGTCTG CTCCGTCAAC TCGTCCAGCC |
| | | TCCTCCTGTG CCGGAGCCCT GCTGTACCAG ACAGAGCCCA CCCGCAGCGG |
| | | GTCTTCTTCA CCCTAGACAA CGTGCAAGTG GACTTCGCCA GTGCCAGTGG |
| | | GGGCCAGGGC TTCCTGTACC AGCCCAACCC CCGCCTGGCA CCCCTCAGCC |
| | | GCGAGGGGCC TGCCCGCCCC TACCGCCTCA AGCCAGGCCA TGTCCTGGAT |
| | | GTGGAGGGCG AGGGCCTCAA CCTGGGCATC AGCAAGGAGG AGGTGCGCGT |
| | | GCACATCGGC CGCGGCGAGT GCCTGGTGAA GACGCTCACG CGCACCCACC |
| | | TGTACTGCGA GCCGCCTGCG CACGCCCCGC AGCCTGCCAA TGGCTCCGGC |
| | | CTGCCACAGT TCGTGGTGCA GATGGGCAAT GTGCAGCTGG CCCTGGGCCC |
| | | TGTGCAGTAC GAGGCTGAAC CCCCGCTGTC TGCCTTTCCC GTGGAGGCCC |
| | | AGGCAGGCGT GGGCATGGGT GCTGCAGTGC TGATTGCCGC CGTGCTCCTC |
| | | CTCACCCTCA TGTACAGGCA CAAGAGCAAG CAGGCCCTGC GGGACTACCA |
| | | GAAGGTGCTA GTGCAGCTGG AGAGCCTGGA GACCGGCGTG GGAGACCAGT |
| | | GCCGCAAGGA GTTCACAGAC CTCATGACGG AGATGACCGA CCTCAGCAGC |
| | | GACCTGGAGG GCAGCGGGAT CCCCTTCCTG GACTACCGCA CCTACGCCGA |
| | | GCGCGCCTTC TTCCCTGGCC ATGGCGGTTG CCCGCTGCAG CCCAAGCCTG |
| | | AGGGGCCAGG GGAGGACGGC CACTGTGCCA CTGTGCGCCA GGGCCTCACG |
| | | CAGCTCTCCA ACCTGCTCAA CAGCAAGCTC TTCCTCCTCA CGCTCATCCA |
| | | CACCCTGGAG GAGCAGCCCA GCTTTTCCCA GAGGGATCGC TGCCATGTGG |
| | | CTTCGCTGCT GTCGCTAGCG CTACACGGCA AGCTGGAGTA CCTGACGGAC |
| | | ATCATGAGGA CCCTGCTGGG TGACCTGGCG GCCCATTACG TGCACAGGAA |
| | | CCCCAAGCTC ATGCTACGCA GGACAGAGAC CATGGTGGAG AAACTGCTCA |
| | | CCAACTGGCT GTCCATCTGC CTGTACGCCT TCCTGAGGGA GGTGGCTGGT |
| | | GAACCACTGT ACATGCTCTT CCGGGCCATC CAGTACCAGG TGGACAAAGG |
| | | CCCCGTGGAC GCCGTGACAG GCAAGGCCAA ACGGACCCTG AATGATAGCC |
| | | GCTTGCTGCG GGAGGACGTG GAGTTCCAGC CCCTGACGCT GATGGTGCTG |
| | | GTGGGGCCCG GGGCTGGCGG GGCCGCAGGC AGCAGCGAGA TGCAGCGCGT |
| | | GCCAGCCCGG GTGCTCGACA CGGACACCAT CACCCAGGTC AAGGAGAAGG |
| | | TGTTGGACCA AGTCTACAAG GGCACCCCCT TCTCCCAGAG GCCCTCAGTG |
| | | CATGCCCTAG ACCTTGAGTG GCGCTCAGGC CTGGCTGGTC ACCTGACCCT |
| | | ATCGGACGAA GACTTGACCT CCGTGACCCA AAACCACTGG AAGAGACTCA |
| | | ACACCTTGCA ACACTACAAG GTCCCAGATG GAGCAACAGT GGGGCTCGTC |
| | | CCTCAGCTGC ACCGTGGCAG CACCATCTCC CAGAGCCTGG CCCAGAGGTG |
| | | CCCCTTGGGA GAGAACATAC CCACGCTGGA GGATGGCGAG GAGGGGGGGG |
| | | TGTGCCTCTG GCACCTGGTG AAAGCCACCG AGGAGCCAGA AGGGGCCAAG |
| | | GTGCGGTGCA GCAGCCTGCG GGAGCGCGAG CCAGCAAGGG CCAAGGCCAT |
| | | TCCGGAAATC TACCTCACCC GTCTGCTGTC CATGAAGGGC ACGCTGCAGA |
| | | AGTTTGTGGA CGACACCTTC CAGGCCATTC TCAGCGTGAA CCGGCCCATC |
| | | CCCATCGCCG TCAAGTACCT GTTTGACCTT CTGGATGAGC TAGCAGAGAA |
| | | GCACGGCATC GAGGACCCAG GGACCCTGCA CATCTGGAAG ACCAACAGTC |
| | | TGCTGCTGCG GTTCTGGGTG AATGCCTTGA AGAACCCACA GCTCATCTTT |
| | | GATGTACGGG TGTCGGACAA TGTGGACGCC ATCCTTGCTG TCATCGCCCA |
| | | GACCTTCATT GACTCCTGTA CCACCTCGGA GCATAAAGTG GGCCGGGATT |
| | | CCCCAGTGAA CAAACTGCTC TACGCCCGGG AGATCCCACG CTACAAGCAG |
| | | ATGGTGGAGA GGTACTATGC GGACATTCGC CAGAGCTCTC CGGCGAGCTA |
| | | CCAGGAGATG AACTCTGCTT TGGCTGAGCT CTCCGGGAAC TACACTTCTG |
| | | CTCCCCACTG TCTGGAGGCT CTGCAAGAAC TCTACAACCA CATCCACAGG |
| | | TACTATGATC AGATTATCAG TGCCCTGGAG GAGGACCCTG TGGGCCAGAA |
| | | GCTGCAGCTG GCCTGCCGCC TGCAGCAGGT CGCCGCCCTG GTGGAAAACA |
| | | AAGTGACTGA CCTGTGAGCT CTGGCTCAGA CAGCAGCAAG CCGGATCCAC |
| | | CAACACCGCA GCGCCTTATG ACCCCGGAAC CGAGCCAGCC ACTGAGGGGA |
| | | GCTGGCAGAG CCTGGGGGCA CAGGGTGCAA AGCCAGGCAC TGTGCCCAGC |
| | | AGTGGGCTCC CTGCCTGCCA CCTCCCCTGC CAGCCCACCC ACCTTCCCCC |
| | | CACCTGAGAT TGTTTCTAAT TTATAAGGAT CCCCCTCCTT CCCCCTCTCC |
| | | CCATTGTATT TATTTGCCTG CTGGAAAATC ACATCCGGAA ATAAAATAGA |
| | | AATATGTCTT TTTATTTTA |
| 50 | POU3F2 | AGAGAGCTGG AGAGAGCAGG GAGAGGGGGG AGCGCCGAGC TAGTCAGAGA |
| | | GTGAGCGAGA GCGAGAAGGA GGGAGAGGAG GAGAAAGAGA GCGAGGGCGG |
| | | GCGGGAGGCG GCGGCGGCGG CAGCAGCAGC AGTAATAGCA GGAGCAGCAA |
| | | CAGAAGGCGT CGGAGCGGGC GTCGGAGCTG CCCGCTGTGG GAGAGAGAGG |
| | | AGACAGAAAG AGCGAGCGAG GAGAGGGAGC CCGAGGCGAA AAAGTAACTG |
| | | TCAAATGCGC GGCTCCTTTA ACCGGAGCGC TCAGTCCGGC TCCGAGAGTC |
| | | ATGGCGACCG CAGCGTCTAA CCACTACAGC CTGCTCACCT CCAGCGCCTC |
| | | CATCGTGCAC GCCGAGCCGC CCGGCGGCAT GCAGCAGGGC GCGGGGGGCT |
| | | ACCGCGAAGC GCAGAGCCTG GTGCAGGGCG ACTACGGCGC TCTGCAGAGC |
| | | AACGGACACC CGCTCAGCCA CGCTCACCAG TGGATCACCG CGCTGTCCCA |
| | | CGGCGGCGGC GGCGGGGGCG GTGGCGGCGG CGGGGGGGGC GGGGGCGGCG |
| | | GCGGGGGGGG CGGCGACGGC TCCCCGTGGT CCACCAGCCC CCTGGGCCAG |
| | | CCGGACATCA AGCCCTCGGT GGTGGTGCAG CAGGGCGGCC GCGGAGACGA |
| | | GCTGCACGGG CCAGGCGCCC TGCAGCAGCA GCATCAGCAG CAGCAACAGC |
| | | AACAGCAGCA GCAACAGCAG CAACAGCAGC AGCAGCAGCA GCAACAGCGG |
| | | CCGCCGCATC TGGTGCACCA CGCCGCTAAC CACCACCCGG GACCCGGGGC |
| | | ATGGCGGAGC GCGGCGGCTG CAGCGCACCT CCCACCCTCC ATGGGAGCGT |
| | | CCAACGGCGG CTTGCTCTAC TCGCAGCCCA GCTTCACGGT GAACGGCATG |
| | | CTGGGCGCCG GCGGGCAGCC GGCCGGTCTG CACCACCACG GCCTGCGGGA |
| | | CGCGCACGAC GAGCCACACC ATGCCGACCA CCACCCGCAC CCGCACTCGC |
| | | ACCCACACCA GCAGCCGCCG CCCCCGCCGC CCCCGCAGGG TCCGCCTGGC |
| | | CACCCAGGCG CGCACCACGA CCCGCACTCG GACGAGGACA CGCCGACCTC |
| | | GGACGACCTG GAGCAGTTCG CCAAGCAGTT CAAGCAGCGG CGGATCAAAC |
| | | TGGGATTTAC CCAAGCGGAC GTGGGGCTGG CTCTGGGCAC CCTGTATGGC |
| | | AACGTGTTCT CGCAGACCAC CATCTGCAGG TTTGAGGCCC TGCAGCTGAG |
| | | CTTCAAGAAC ATGTGCAAGC TGAAGCCTTT GTTGAACAAG TGGTTGGAGG |
| | | AGGCGGACTC GTCCTCGGGC AGCCCCACGA GCATAGACAA GATCGCAGCG |
| | | CAAGGGCGCA AGCGGAAAAA GCGGACCTCC ATCGAGGTGA GCGTCAAGGG |
| | | GGCTCTGGAG AGCCATTTCC TCAAATGCCC CAAGCCCTCG GCCCAGGAGA |
| | | TCACCTCCCT CGCGGACAGC TTACAGCTGG AGAAGGAGGT GGTGAGAGTT |
| | | TGGTTTTGTA ACAGGAGACA GAAAGAGAAA AGGATGACCC CTCCCGGAGG |
| | | GACTCTGCCG GGCGCCGAGG ATGTGTACGG GGGGAGTAGG GACACTCCAC |
| | | CACACCACGG GGTGCAGACG CCCGTCCAGT GAACTCGAGC TGGGGGAGGG |
| | | GCAGAGCGCG GGGCTCCCCC TCCCCTTCGG TCCTTGGCCC TTTCCCGGCC |
| | | CTCTTGTTCC CTCTCTAACT TCTGATTGTT CTTTTATTTT TAATTATTAT |
| | | TTCCCCGTCC CTTAAAAAGA CAAAAAAAAT AAGGCAAAAG GAAAGCAACT |
| | | AAGACACTGG ACTATCCTTT AAAGGTAGCA GGTGTAATGA TGTGTTTTGA |
| | | CCTTTGCAGG CGAGTAACCA GGCAATGGAG TGGAGTGTCT CCTGGAGAGA |
| | | GTGAGGAGAG TGTGTGATAG CTAGAAAGAG AGAGAGACAG AGAGATGGCA |
| | | AGCACTGAGA TAAATACCTG GCAAAACTAA ATAAATTACC AAAAAGGAAA |
| | | AAAAATCCAC CAAACCATGA TAAACACAAA ATGCAGCTTC CTGATGCTTA |
| | | GAGTTGGCAC ATGCTGCTGT GTTTATTTAT TGTGGATTCC CATCAGGAAA |
| | | GAGGAAAAAA TACACATGTT CTTTCATATA GGCAAAATTT AACCACATAA |
| | | ATTTGCACTG CAAGAAAATT GAAGTTTACG TGAACAAATT CATGAGCATA |
| | | TTTTCTCTTT CTCCCCACCG TTAATTTGGG AGTTGCCGTT TTGGGGGATT |
| | | TTGTTTTGCT TTGCTTTATT CATCGGAGAG AGTTGAAGCC AGCTCTTGGC |
| | | CACTCTCCAT TTCTAATGTT CTTGTGTTGC CCCTTCTTCG TACTGTTTGT |
| | | GAACTTTGGT TACCTTCACA TTCCCCTTAC GAGGGTGTAA CATCTATTTG |
| | | TTCCTCTTAC CAAAGCAAAA GGATTGGCTT CATACAAAAT AGACAATTCT |
| | | CTGATTTCAG GAAATGTGCA TGGTCTACCC GCTTTATCGA AGGCAAGAAT |
| | | CCGGTTTGGA ATATAAAAAT AAGCATTGGT TGTTCTTACC AGCCACAAAG |
| | | TAAACTTCAT TTTCAGGCAG TGTTTCTGGG GGAGGTTATG GAGGGAAGAA |
| | | AAAAGAAAAA TCGATAGTGA GTGACTGATT GCTTCATTTT ATCAGGCGGG |
| | | CCCATTGTGA AAGAGCTCAG GGGAAATGTG GAGGTTAAAT ATATTTCCAG |
| | | AGTIGTCCAG CAGAAAGAAA GTGGCACTTT GAAGAGAACT AGGGAAGTAC |
| | | ATATCTTCAG ATATCCCTAT ATAGTTCTCT ACCTTCAGTT TTAGTAACAA |
| | | TTATGAAGAA TTATTTGTGC TGACAGCAGC AGTTAAACTT TGTTTCTCTA |
| | | ATAGCTTTTT TTTTACATAA AAAAAGACCC AGGAACTTAA TAGTGTATGC |
| | | ATAAGACTGT GTTTTTTAGC ACACAGATAC CCACAGCATA CACTGACGAT |
| | | CTCCACGCAG TAGACAGGTT TTGTCTTCAC TAGCTCATTT GTTTATCAAG |
| | | TCATATTTAG GGTCCCACAC CCTCTTTTCC TGTAATTTAT TGCAGAATAC |
| | | ACCACTTTGA CTTGGACAGC TTTCTGCCCC CTCTTTCACT AAGGAAGGCA |
| | | AATGAAGTGA AAAAAAAAAA TGCCATTTTC AATCCTTCCT TTCTCCCCTT |
| | | TGTTAATAGT TTTAAGTGAA TTTTTGACCT TATCTTAATG GAAAACGGTT |
| | | AACTCCAAAC ACAAAAGACT CTACTGGAAA GTGTAGGTGA AAAAACTTGT |
| | | AACTGTATTG AAAATAAATA CCATTAAACT GTGATCAGTT AAAATTTAAA |
| | | AGAAAAATCA GCACAAAAGG GCGCTAAAAG GGAAAACACT TTTTATTAAT |
| | | CTTAAAAGTT TGGGGGTTTT TTTCCAGTTA GGTATTAGAT AAATTTTTAT |
| | | TTTAAAAAAT GAAAGTCTCA CTACCATAAA ATTATGGTTC AGCATCAGAT |
| | | TAGCATTGCA CTCAGTAGTC TTTAAGGTTT TAGGAAATAT GCTTTATATT |
| | | GTCTTTTCAA ACACCTGTGA TTGTTTCATT TTCCATGTTT TTGCAAGATA |
| | | AATGGTGACT TATAATGGGC ATATTTATTT GCCTGTATTT CATTTCCCCC |
| | | AATGAATGTC ACAAGGAGAT GGGCACGGAG CTGCTTCGGG TGCATCACGC |
| | | TGCTCGTTCC TGAGGTATGG GAACTGGCCT TTAGTGAAGC TATCCAGAGC |
| | | AGGGCAAATA GCCACTGGTA AAGGGAGGAA ATGAATTTCC AGATACTTAT |
| | | TACCAAGTAG GTAAGGTCAG AAGCTGGAGT TCAGAGAATG TGTCTACAGC |
| | | TTCTCTGACT CTTATAGGTT TACTAAGATG AAAGTTACCA CTGAACCTTA |
| | | CCACTATGTA TATATGTTTA ATATCTGTCT TTTGAAATGC AGAAATAGTT |
| | | TAAATGTTTC TTTGTCTATT TTTCTTTTTT TTTAATGCTA CCCAGGGAAA |
| | | TATTTTCATA TCATTTTTAA GTGGCCTGCC TCAATGTATA TTTATTTCTT |
| | | TTGAAACAAA AAGGTTCTGG AAACTGTTTT TCTGTAGCTT TAAATGAATA |
| | | GGTGAGCAAA ATCTATATGG GATGTAATTT TTTTGTTCAG TCTCTTAAAA |
| | | AATACTTTGT TTTGGTACAT TTGGTTGTGC TTGTGGGGAA AATAAAAACG |
| | | CAGAGATCCT TATATATTTA TGTTAAAGTA ATATTTTATT ATCTACATAA |
| | | AACAGAAATG CACAATACCT TCATAGTTTG TTCTAATTAT TGAAATATCT |
| | | TTATTTTATT TTTAAAGATA GTGCCAAGTT TTAAGGGGGG AAAACCCTAG |
| | | ACCTTAAATT GACTGAGTTG AGTTGTGTGT AAAACACTTC CCTTCCTTTA |
| | | TACTTCATAA AGTTTTGGAA TAAATTTTAT GCATATACTG CCAGATTTGA |
| | | TGTTCATAAC TTTCAGAGGC TTTTTTTTTT TTTAATGGAG ACTACTGGTC |
| | | TAATTCACTT TACTTTGCAA AAACTATCAG TCCCAAATCT TTCAGTCACT |
| | | ATGCCTGTAG CATTAAATTG AAATGGTCAT TGGGTTTGAG CTTCAATTTG |
| | | CTTGCCATTT CATGGTCCTA CAAAGAGATG TTTGTCCCCT TTAAACATAT |
| | | GCAGATATGC CTGCATCTTC TTTCCAGGTT AACTATACCT TATTCGATTG |
| | | TGCTGTTTTG GAGAACATCA GTGGAACTAG GTGGACTTTG ATCTCTACCC |
| | | ATAGGTCCTC AAATAATTTG GGATCTACAG AAAAGCAGGA TTTAGTAGAT |
| | | TATTATTTTT AATAATTTAC AAATATCTCT TAACAAAGAA TAACCCTGAT |
| | | AGTATACTAT TGTGAGTTTA CTAAATGATT AGAAGTAGAT CCTATACATT |
| | | ATTCCTGTTT GGTTTGCATA AAAAGATGAA TTTTA |
| 51 | POU6F2 | CCCTTGCTTT TGGTGATGGT TGTGAGTGCA GAAGTTGATT GACAGATGCA |
| | | TGCCAGAAAC CCCCATTCTC CTTTTCAAAG ACAACATATG ATGGATTGCT |
| | | ATCTCTCAGC GCAGCAGGAC ACGGGGACCA TGCAAGCTGT AATTGGTCAG |
| | | GCATGAACTC TCTTGGGTTG TGTTACCCTG TTTGCTGTTT ACTGTCAAGA |
| | | TGCTGAAAGA ATGTTCTTAT AATGATCCAA GAGGAAGTGG CAAATGAGTG |
| | | CTCTTCTTCA GGATCCAATG ATAGCTGGAC AAGTCAGTAA GCCCTTGCTG |
| | | TCAGTGCGGA GTGAAATGAA TGCGGAGTTG AGAGGTGAGG ACAAGGCTGC |
| | | TACTTCAGAC AGCGAGCTGA ATGAGCCCCT GCTTGCGCCT GTGGAATCAA |
| | | ATGACAGCGA GGACACTCCC AGCAAGCTCT TCGGGGCTAG AGGAAACCCA |
| | | GCATTATCAG ACCCAGGCAC TCCTGACCAA CACCAGGCCA GTCAGACCCA |
| | | CCCCCCATTT CCAGTTGGGC CACAGCCACT TCTGACGGCA CAGCAGTTAG |
| | | CTTCTGCTGT GGCCGGCGTG ATGCCGGGAG GCCCCCCAGC CCTCAACCAG |
| | | CCAATCCTCA TTCCCTTCAA CATGGCGGGA CAGCTAGGAG GCCAGCAAGG |
| | | ACTGGTTCTC ACACTGCCAA CAGCGAATCT CACCAACATC CAAGGGCTGG |
| | | TGGCAGCAGC TGCAGCCGGA GGCATTATGA CTCTGCCACT GCAAAATCTA |
| | | CAAGCTACCT CATCCCTGAA CTCCCAGCTC CAGCAGCTCC AGCTCCAGCT |
| | | CCAGCAGCAG CAGCAGCAGC AGCAGCAGCA GCCTCCCCCG TCAACCAACC |
| | | AGCACCCGCA ACCAGCCCCA CAGGCGCCCT CGCAGTCCCA GCAGCAGCCG |
| | | CTGCAGCCCA CCCCACCCCA GCAGCCACCA CCCGCCTCTC AGCAGCCGCC |
| | | AGCTCCTACA TCTCAGCTGC AACAGGCGCC TCAGCCCCAG CAGCACCAAC |
| | | CCCACTCCCA CTCCCAGAAC CAGAACCAAC CATCTCCAAC CCAGCAGAGC |
| | | TCCAGCCCCC CGCAGAAACC TAGTCAGTCT CCAGGACATG GCCTGCCTTC |
| | | ACCGCTCACG CCACCCAATC CTCTACAGCT GGTTAATAAT CCACTAGCAA |
| | | GTCAGGCTGC AGCGGCTGCA GCAGCCATGA GCTCCATAGC AAGCTCACAG |
| | | GCCTTTGGCA ATGCCCTCTC CAGTCTTCAG GGGGTCACAG GTCAACTAGT |
| | | TACTAATGCA CAAGGACAGA TTATCGGGAC CATTCCACTG ATGCCTAATC |
| | | CAGGGCCATC GAGCCAAGCA GCAAGCGGCA CTCAGGGCTT GCAAGTGCAG |
| | | CCAATCACCC CCCAGCTCCT CACAAACGCC CAGGGCCAGA TCATCGCCAC |
| | | AGTCATTGGG AACCAGATCC TGCCCGTGAT CAACACCCAG GGCATCACGC |
| | | TGTCACCCAT CAAGCCCGGC CAGCAGCTCC ACCAACCCTC CCAGACGTCA |
| | | GTGGGTCAAG CAGCCTCCCA AGGCAACCTT CTGCACCTGG CTCACAGCCA |
| | | AGCATCCATG TCTCAAAGTC CCGTCCGGCA GGCTTCCTCT TCTTCCTCCT |
| | | CATCCTCCTC TTCTTCAGCT TTGAGCGTGG GCCAGTTAGT CAGCAATCCT |
| | | CAAACGGCAG CGGGTGAGGT GGATGGGGTT AATCTGGAGG AGATCCGAGA |
| | | ATTTGCCAAA GCTTTTAAAA TCCGGCGCCT GTCCCTTGGC CTGACCCAGA |
| | | CTCAGGTGGG ACAGGCTCTC AGTGCTACAG AGGGCCCCGC GTACAGCCAG |
| | | TCGGCCATCT GCAGACACAC CATCCTGAGA AGCCACTTTT TCCTACCACA |
| | | GGAAGCCCAA GAGAACACTA TAGCTAGCAG TCTGACAGCC AAACTGAACC |
| | | CTGGCCTTTT GTATCCTGCC AGGTTTGAAA AGCTGGACAT CACCCCTAAA |
| | | AGTGCCCAGA AGATCAAGCC GGTGCTTGAG CGGTGGATGG CTGAGGCTGA |
| | | GGCCCGCCAT CGAGCAGGTA TGCAGAACCT GACCGAGTTT ATCGGGAGTG |
| | | AACCATCCAA AAAGCGCAAG CGGCGCACCT CCTTCACACC CCAGGCCCTT |
| | | GAGATCCTCA ATGCCCACTT TGAGAAGAAC ACACACCCTT CTGGGCAGGA |
| | | AATGACCGAA ATTGCTGAGA AGCTGAACTA TGACCGAGAA GTAGTTAGAG |
| | | TTTGGTTCTG CAATAAGAGG CAAGCCCTGA AGAACACAAT TAAACGCTTA |
| | | AAACAGCACG AGCCGGCCAC GGCAGTCCCT TTGGAGCCCT TAACAGACTC |
| | | TCTGGAAGAA AACTCCTAAA GAGATGCCCA CCCATAATCA GAAGCAAAAT |
| | | TCACAGAAAC TAAACTCCAC CCTTGGGACT CCACAACAAC AACAACAACA |
| | | AAATTTAATT TAATTTAAAA ATAGCCCCAG TCGTCATCAC CCTTGTAAGT |
| | | AAATGACTAA GAAAACTACC AAGTGGACAG AATGGTTTCT ACATGTCCGT |
| | | TGGTTTTCCA AAAAGGAAAG AAGAAAATTT TTAGAAAATT TTTAAACAAG |
| | | GAATACACCA CACTGAAGGT GTGTGTGGTA GGATAGTTCC CTTCCCCCAC |
| | | CTGTCTCCCC CAAAGCCAGT TTTTTAATGG ACTTAAAGCA AACCAAATAA |
| | | CCACGTACTT TTTTCTGTAT ATTATGAAAA TGTGAACACA TTTTAAGGAA |
| | | AAAGAAAAAA AAAAACTAAA CCAAAAACCA ACAACGAAGG AACAAAAACT |
| | | TTGATCTGTT CAAAGCGAAT ACAAGCCTGC CACCTGGAGG AAGGACTGCA |
| | | CCCCTTCAGG TACTAAGTGC TGATTCACTA TGAAACCTAT TAACCAAAGT |
| | | CAGAAACATG GCATTGCAAC GCGATCGTTC GTCTACGCTT CTCCGCACGT |
| | | AAAGTTGTGT TACGAATTTT TACATTTGTA CTAACAGAAC AATAGGAAGC |
| | | CTGATTTCTC CCATCTTTCC CATCTACTGT CTCCACCCAT GGGGTGGGTA |
| | | CCATTGTTGA AGCCATTCTG TGAGGCTCAC TATTGGGTTT TTTTGTGGGG |
| | | GTGGTAGGGA GGGTGGTCTT TTTTCTTTGT CTTTCTTTTT GACGGGAGGG |
| | | CATCCTGGAT CGTGTGCCAA AGCATTTGTT GCTTTTTTCT CACTATGACT |
| | | TGTGGGTTTG AGAAAAGAAA ATGGAGCTCG CATTTCTCTC TTTTCCTCCA |
| | | TTTCTCCATC TCCCTCGCGC GTGGCTCCTG GGGTCTGCTG GAAGGCCACA |
| | | GAAGTGGGGA GAAGCAGTGT CTTTTCCACT CAGATCCTAG TGAAATGCAG |
| | | GAGAGACTCC AAAATAACTA GGGCTTTGCT CGATGAACTG TCAACACTGG |
| | | CATAAGCTGT AATTGTGCTC ACTGTCCACA CCAGAGCTTG GGATTTTTCT |
| | | CAGTCTGTTG GCCACGTACA TGGAGAGCTG ACCAAAACTA ATTTTGTAAT |
| | | ATAAACATAA GCTGCACATT TGGTTCAATA CTTACATCTA TGTTATGCTT |
| | | CTGTGCAAAG CAATTTCTCT CAGAAGTCTG ATAGCCAAAG AAATGTCTCA |
| | | AGATTTCAGT CAAATACACA CATGACATGC ACACACCCAT AAACACACAC |
| | | ACAAAGAAAC AGGCTAAAAA GAAAGTGATA GCAACTGGAT CATTAAAGGC |
| | | CATCGTGTAT CCTGTTAATC TCATCTTTTC TGCTACTTCT TTCATTCCAA |
| | | TAGCGATGGC AGACTTTTTT CAGGGGGAAT AATTACTCTT CGGGATATAT |
| | | AAATGGAGCA GAAAGGTGGC GGGCAAATTT AAACAACTGG AATTGGGGGC |
| | | CAGGTTCTTC AGGGGAAAGG AAGTTTGAAG AAGCTTTCAC CAAAAGAAAA |
| | | AAATATAGAA GGGGCTTATC TAACAATCAG AATAGCCTGC AATATGAGAA |
| | | CTAGAGGATT ATTTCCTTTA AAAAAAATAA CTTAAAAGAT CTGTGGGCCA |
| | | CAATAACCTA ATAAGCAAGG ACATGTTGGG ATGTGAGGAC GGCTGTGCAG |
| | | GTGAGCACTT GGCAGCTGCC ACTGGTGTTT TCACCCAGGT ACAGGCAGAC |
| | | GGTGTTCCAT TAAAGGAGCA GGCAAAAGAG AGCTGTCAAT TTGCTTGCTG |
| | | TTGAAATGTA CATATTTATG CATAATTTAT AATCATGCTA ATGTATTATC |
| | | TAGAAGTAAG TTGTGAAAGA AAAAAGAGAA ACCCAATCTC AAGTTGCCCA |
| | | ACTGATTAGA AATGCCATCT CCTTCCCCTA ATACACTCCC TCTCCCTGGG |
| | | GAGTTTGAGT TTCTTCTGTT GGCTCCTTTG CTGTTTGTGT AAATGCTACA |
| | | GTATAATTGT CCCCTCCAAT GATTCATGCA GCAATCAAGA GACTACCAGC |
| | | AAGGAAACTC TAAAAGGAAT CCTGGAAGTG GAACAAGTCA CTCGTAAAGG |
| | | TGTTTCCATT TTACCAGTCA ATGCTAACTG GACCACAGCT AACTTGATCT |
| | | TCAATGCCAA CATCACAGAG CCCCTTTCGC CACCTCAAAA ACTGCACAAA |
| | | CATGGACAAC CCAGAAACCC AAAACTTTAT TGATTAAAAC AGATTTTGGA |
| | | TTCTCCTAGG ACCTGCTCCA AATTCCATCA AGAAAAGCTC CCTGCGAAGA |
| | | GAAGTTAACA AGATAACATA TGATGGATGC TAAATATTTA AATATATGCC |
| | | GGCAGCGGTT ACATAAGGCC TGCTCAGTTC TGAGATCTAT AATTGGGAGG |
| | | GTCTAACTAG GACAGGGAAA ATATAAAATA AAAGTAATCT TCCAGAGTGG |
| | | AACAAAAAAG AAATATATTC ATCCGACAGT CGTAACACCA TTCATGTGCA |
| | | GTGATTTTTT TATAGTTTTA TAATTTTGTA TTGTTTTATA AATTATTTAT |
| | | AAGGTGTTGT AATGCTTCTT ATATTAATTT TTTACAGATA AATTTTTTGC |
| | | TACAAGGCAT AAAAAGGTGC CTGAACCGAT TCTTAGGAAT ATAAATTATA |
| | | CTGTGTAACT CATAAGTCTT TGGGACCACA CCTAATGCTT AATTTTATGA |
| | | TGACATTTCT CATGTCTACT TGTAAACAAC TTCATATGCC AATGGCGTTT |
| | | AAGTGTCTTT TATGTTTCCA TGAACTGAAG GTTAAGGTTG CCACCCACAA |
| | | AAAATAAAAG CCACTTTGGG CATATGTGAT TGTATTTCAA GCTTCAGTAT |
| | | TTTTCTCACA TAATTTTTAA TTATTGTTAT CTGCATTTTC ATTACTTCAT |
| | | GAAAAAATGA CAGTGGATAT TACATTTTAC ACTTGTTTAT AGATTTTCTT |
| | | TCTTTTTCAA CCAAGAAAAT ATTTTGAACA ATTGAAACTT TTAATTGCAT |
| | | TTGCAAGGTT TTGGCTTCTT TATGTTGTCA TCACACAGAT GCACACACAC |
| | | ATACACATAC AACACAAAAG AGAGAACAAA AAAGCAAAAG GAGCTAGGAA |
| | | AAAAACAAAA CAAAACAAGT AGAGGTGTAT CAAAGAACTC AAGCTATAAC |
| | | CAAAAAGAAA TCGTAAAATG CCTTTGCTCG TTTCTCTACG CTGGACCAAA |
| | | GCTCAATATT TGTAGGTATA TGCACATTGT ATAGATATGG CTAAATGTTG |
| | | CTGACAATCT CGCAATACTA AACTGTTCCT ATTTTAAGAA AAAAAAAAGA |
| | | AATACAAACT GTTCATCAAT GTTTTACCTC AGCACTCTAC TTGTACCCAG |
| | | TTAATGACCA AGCTTAAAAA AAAATTGAGA TAAAGGAAAT TGATTTTCAT |
| | | TTCAATGTTT GACTGTAAAA TCTGTTTGGA TAACATTTTG TAATGAGCTT |
| | | TTTGTCATGT GATTTGCTTG TCTTCAACTT GAAATTATGT GAGGCACATT |
| | | TGTTTATTTG TTGTTAAGAA AGTGATTTTT TTTTTTTTTT GTCCTATGTG |
| | | CTGTGATCTA GACTGGTCAC CGGGGTCACT TATGAGGCAC CACAAAGAGA |
| | | TCTGCTTCTC CGTGCCCGGA GCAGGCAGCA GGAGGGAGGG CAGGAAACAG |
| | | GACTGGGTTG CTTTGGAAAA ATCATCATGA CAAGAAGTTG ACATGATAGA |
| | | CTTGTGACCA AGAAGCCAAA CTGAATATTT AAAAGCTCCT TACTTGCTCT |
| | | GACATTGAAA CCAAAGCTGA TTTTATCTGC ACAGGTTGCT TAATATTTAA |
| | | AAAAAAAAAA AACTGTACTT AATCTAGAGC AATATCTGTA TGGTCAGTAA |
| | | AGCTGCACTT TGTGTATTTC TTAACAGCTT CAGATCTGTC ACTTTTAATT |
| | | TGTACCATAA AAAATAAATA ATTGTTTGAC ATGA |
| 52 | SOX10 | AGTCGCTCAG TCAGTCTCGG GCTGTCCGGC CAGGGTGGTT GGTGGTAAGG |
| | | ATTCAGGCTC CGTCCTAACG AGGCCGTGGC CTGAGGCTCA GGGCCCCCCG |
| | | CCCCTCCCTC CCAGCCCACC AGCGTCACCT CCCAGCCCCG AGCTGGACCG |
| | | CACACCTTGG GACACGGTTT TCCACTTCCT AAGGACGAGC CCCAGACTGG |
| | | AGGAGAGGTC CGAGGAGGTG GGCGTTGGAC TCTTTGCGAG GACCCCGGCG |
| | | GCTGGCCCGG GGGAGGCGGC CGAGGCGGCG GCGGCGGCGG CCGGGGGCGA |
| | | CATGGCGGAG GAGCAGGACC TATCGGAGGT GGAGCTGAGC CCCGTGGGCT |
| | | CGGAGGAGCC CCGCTGCCTG TCCCCGGGGA GCGCGCCCTC GCTAGGGCCC |
| | | GACGGCGGCG GCGGCGGATC GGGCCTGCGA GCCAGCCCGG GGCCAGGCGA |
| | | GCTGGGCAAG GTCAAGAAGG AGCAGCAGGA CGGCGAGGCG GACGATGACA |
| | | AGTTCCCCGT GTGCATCCGC GAGGCCGTCA GCCAGGTGCT CAGCGGCTAC |
| | | GACTGGACGC TGGTGCCCAT GCCCGTGCGC GTCAACGGCG CCAGCAAAAG |
| | | CAAGCCGCAC GTCAAGCGGC CCATGAACGC CTTCATGGTG TGGGCTCAGG |
| | | CAGCGCGCAG GAAGCTCGCG GACCAGTACC CGCACCTGCA CAACGCTGAG |
| | | CTCAGCAAGA CGCTGGGCAA GCTCTGGAGG CTGCTGAACG AAAGTGACAA |
| | | GCGCCCCTTC ATCGAGGAGG CTGAGCGGCT CCGTATGCAG CACAAGAAAG |
| | | ACCACCCGGA CTACAAGTAC CAGCCCAGGC GGCGGAAGAA CGGGAAGGCC |
| | | GCCCAGGGCG AGGCGGAGTG CCCCGGTGGG GAGGCCGAGC AAGGTGGGAC |
| | | CGCCGCCATC CAGGCCCACT ACAAGAGCGC CCACTTGGAC CACCGGCACC |
| | | CAGGAGAGGG CTCCCCCATG TCAGATGGGA ACCCCGAGCA CCCCTCAGGC |
| | | CAGAGCCATG GCCCACCCAC CCCTCCAACC ACCCCGAAGA CAGAGCTGCA |
| | | GTCGGGCAAG GCAGACCCGA AGCGGGACGG GCGCTCCATG GGGGAGGGCG |
| | | GGAAGCCTCA CATCGACTTC GGCAACGTGG ACATTGGTGA GATCAGCCAC |
| | | GAGGTAATGT CCAACATGGA GACCTTTGAT GTGGCTGAGT TGGACCAGTA |
| | | CCTGCCGCCC AATGGGCACC CAGGCCATGT GAGCAGCTAC TCAGCAGCCG |
| | | GCTATGGGCT GGGCAGTGCC CTGGCCGTGG CCAGTGGACA CTCCGCCTGG |
| | | ATCTCCAAGC CACCAGGCGT GGCTCTGCCC ACGGTCTCAC CACCTGGTGT |
| | | GGATGCCAAA GCCCAGGTGA AGACAGAGAC CGCGGGGCCC CAGGGGCCCC |
| | | CACACTACAC CGACCAGCCA TCCACCTCAC AGATCGCCTA CACCTCCCTC |
| | | AGCCTGCCCC ACTATGGCTC AGCCTTCCCC TCCATCTCCC GCCCCCAGTT |
| | | TGACTACTCT GACCATCAGC CCTCAGGACC CTATTATGGC CACTCGGGCC |
| | | AGGCCTCTGG CCTCTACTCG GCCTTCTCCT ATATGGGGCC CTCGCAGCGG |
| | | CCCCTCTACA CGGCCATCTC TGACCCCAGC CCCTCAGGGC CCCAGTCCCA |
| | | CAGCCCCACA CACTGGGAGC AGCCAGTATA TACGACACTG TCCCGGCCCT |
| | | AAAGGGGGCC CTGTCGCCAC CACCCCCCGC CCAGCCCCTG CCCCCAGCCT |
| | | GTGTGCCCTG TTCCTTGCCC ACCTCAGGCC TGGTGGTGGC AGTGGAGGAG |
| | | GCTGAGGAGG CTGAAGAGGC TGACAGGTCG GGGGGCTTTC TGTCTGGCTC |
| | | ACTGCCCTGA TGACCCACCC GCCCCATCCA GGCTCCAGCA GCAAAGCCCC |
| | | AGGAGAACAG GCTGGACAGA GGAGAAGGAG GTTGACTGTT GCACCCACAC |
| | | TGAAAGATGA GGGGCTGCAC CTTCCCCCAG GAATGACCCT CTATCCCAGG |
| | | ACCTGAGAAG GGCCTGCTCA CCCTCCTCGG GGAGGGGAAG CACCAGGGTT |
| | | GGTGGCATCG GAGGCCTTAC CACTCCTATG ACTCCTGTTT TCTCTCTCAC |
| | | AGATAGTGAG GGTCTGACAT GCCCATGCCA CCTATGCCAC AGTGCCTAAG |
| | | GGCTAGGCCA CCCAGAGACT GTGCCCGGAG CTGGCCGTGT CTCCCACTCA |
| | | GGGGCTGAGA GTAGCTTTGA GGAGCCTCAT TGGGGAGTGG GGGGTTCGAG |
| | | GGACTTAGTG GAGTTCTCAT CCCTTCAATG CCCCCTCCCT TTCTGAAGGC |
| | | AGGAAGGAGT TGGCACAGAG GCCCCCTGAT CCAATTCTGT GCCAATAACC |
| | | TCATTCTTTG TCTGAGAAAC AGCCCCCAGT CCTCCTCCAC TACAACCTCC |
| | | ATGACCTTGA GACGCATCCC AGGAGGTGAC GAGGCAGGGG CTCCAGGAAA |
| | | GGAATCAGAG ACAATTCACA GAGCCTCCCT CCCTGGGCTC CTTGCCAGCT |
| | | CCCTCTTCCC TTACTAGGCT CTATGGCCCC TGCTCAGTCA GCCCCACTCC |
| | | CTGGGCTTCC CAGAGAGTGA CAGCTGCTCA GGCCCTAACC CTTGGCTCCA |
| | | GGAGACACAG GGCCCAGCAC CCAGGTTGCT GTCGGCAGGC TGAAGACACT |
| | | AGAATCCTGA CCTGTACATT CTGCCCTTGC CTCTTACCCC TTGCCTCCCA |
| | | GTGGTATTTG AATAAAGTAT GTAGCTATAT CTGCCCCTAT TTTCCTGTTC |
| | | TGCAGCCCCC CAAATCCACA TGTAACTCAT TACTGTCTCC TGTTATTTAT |
| | | CTCAGTAGTC CCCTCTCCTA GCCACTCTAG CCCCTATTAA CTCTGCATTA |
| | | AGCATTCCAC ATAATAAAAT TAAAGGTTCC GGTTA |

In some embodiments, the cell types disclosed in the supplementary figures express RNA associated with the accession number in Table 4. In some embodiments, the cell types disclosed in the supplementary figures express protein associated with the accession number in Table 4.

TABLE 4
Genbank Accession Numbers

| From | To | Species | Gene Name |
|---|---|---|---|
| STMN2 | DQ895968 | Homo sapiens | stathmin 2(STMN2) |
| STMN2 | AK308557 | Homo sapiens | stathmin 2(STMN2) |
| STMN2 | AK297485 | Homo sapiens | stathmin 2(STMN2) |
| STMN2 | AK297594 | Homo sapiens | stathmin 2(STMN2) |
| STMN2 | AL110174 | Homo sapiens | stathmin 2(STMN2) |
| STMN2 | DQ892719 | Homo sapiens | stathmin 2(STMN2) |
| STMN2 | NM_007029 | Homo sapiens | stathmin 2(STMN2) |
| STMN2 | S82024 | Homo sapiens | stathmin 2(STMN2) |
| STMN2 | XM_005251142 | Homo sapiens | stathmin 2(STMN2) |
| STMN2 | BT020034 | Homo sapiens | stathmin 2(STMN2) |
| STMN2 | AI096716 | Homo sapiens | stathmin 2(STMN2) |
| STMN2 | NM_001199214 | Homo sapiens | stathmin 2(STMN2) |
| STMN2 | BQ069488 | Homo sapiens | stathmin 2(STMN2) |
| STMN2 | DC319376 | Homo sapiens | stathmin 2(STMN2) |
| STMN2 | BC006302 | Homo sapiens | stathmin 2(STMN2) |
| STMN2 | AK292737 | Homo sapiens | stathmin 2(STMN2) |
| STMN2 | AK309130 | Homo sapiens | stathmin 2(STMN2) |
| STMN2 | D50375 | Homo sapiens | stathmin 2(STMN2) |
| STMN2 | AK091336 | Homo sapiens | stathmin 2(STMN2) |
| STMN2 | AK299500 | Homo sapiens | stathmin 2(STMN2) |
| STMN2 | CR456833 | Homo sapiens | stathmin 2(STMN2) |
| AQP4 | AB209156 | Homo sapiens | aquaporin 4(AQP4) |
| AQP4 | BI917845 | Homo sapiens | aquaporin 4(AQP4) |
| AQP4 | AB128929 | Homo sapiens | aquaporin 4(AQP4) |
| AQP4 | CD105892 | Homo sapiens | aquaporin 4(AQP4) |
| AQP4 | BC022286 | Homo sapiens | aquaporin 4(AQP4) |
| AQP4 | BI667387 | Homo sapiens | aquaporin 4(AQP4) |
| AQP4 | AK026728 | Homo sapiens | aquaporin 4(AQP4) |
| AQP4 | BC030745 | Homo sapiens | aquaporin 4(AQP4) |
| AQP4 | AV725241 | Homo sapiens | aquaporin 4(AQP4) |
| AQP4 | R35726 | Homo sapiens | aquaporin 4(AQP4) |
| AQP4 | N50070 | Homo sapiens | aquaporin 4(AQP4) |
| AQP4 | AK222684 | Homo sapiens | aquaporin 4(AQP4) |
| AQP4 | XM_011525942 | Homo sapiens | aquaporin 4(AQP4) |
| AQP4 | AK295069 | Homo sapiens | aquaporin 4(AQP4) |
| AQP4 | CK001094 | Homo sapiens | aquaporin 4(AQP4) |
| AQP4 | AL138136 | Homo sapiens | aquaporin 4(AQP4) |
| AQP4 | KF055862 | Homo sapiens | aquaporin 4(AQP4) |
| AQP4 | NM_001650 | Homo sapiens | aquaporin 4(AQP4) |
| AQP4 | NM_004028 | Homo sapiens | aquaporin 4(AQP4) |
| AOP4 | BI596912 | Homo sapiens | aquaporin 4(AQP4) |
| AQP4 | D63412 | Homo sapiens | aquaporin 4(AQP4) |
| AQP4 | U34845 | Homo sapiens | aquaporin 4(AQP4) |
| AQP4 | BC045780 | Homo sapiens | aquaporin 4(AQP4) |
| AQP4 | HQ447648 | Homo sapiens | aquaporin 4(AQP4) |
| AQP4 | HQ901095 | Homo sapiens | aquaporin 4(AQP4) |
| AQP4 | NM_001317387 | Homo sapiens | aquaporin 4(AQP4) |
| AQP4 | U63622 | Homo sapiens | aquaporin 4(AQP4) |
| AQP4 | U63623 | Homo sapiens | aquaporin 4(AQP4) |
| AQP4 | AL119338 | Homo sapiens | aquaporin 4(AQP4) |
| AQP4 | NM_001317384 | Homo sapiens | aquaporin 4(AQP4) |
| AQP4 | BQ638704 | Homo sapiens | aquaporin 4(AQP4) |
| AQP4 | BU687682 | Homo sapiens | aquaporin 4(AQP4) |
| STMN4 | AK309207 | Homo sapiens | stathmin 4(STMN4) |
| STMN4 | AK294023 | Homo sapiens | stathmin 4(STMN4) |
| STMN4 | AL136568 | Homo sapiens | stathmin 4(STMN4) |
| STMN4 | BC011520 | Homo sapiens | stathmin 4(STMN4) |
| STMN4 | AI638208 | Homo sapiens | stathmin 4(STMN4) |
| STMN4 | DQ894219 | Homo sapiens | stathmin 4(STMN4) |
| STMN4 | AK225142 | Homo sapiens | stathmin 4(STMN4) |
| STMN4 | NM_030795 | Homo sapiens | stathmin 4(STMN4) |
| STMN4 | XM_005273652 | Homo sapiens | stathmin 4(STMN4) |
| STMN4 | BC111001 | Homo sapiens | stathmin 4(STMN4) |
| STMN4 | XM_005273655 | Homo sapiens | stathmin 4(STMN4) |
| STMN4 | AK295329 | Homo sapiens | stathmin 4(STMN4) |
| STMN4 | AJ303455 | Homo sapiens | stathmin 4(STMN4) |
| STMN4 | NM_001283054 | Homo sapiens | stathmin 4(STMN4) |
| STMN4 | DQ891040 | Homo sapiens | stathmin 4(STMN4) |
| STMN4 | NM_001283053 | Homo sapiens | stathmin 4(STMN4) |
| STMN4 | AK297415 | Homo sapiens | stathmin 4(STMN4) |
| STMN4 | NM_001283055 | Homo sapiens | stathmin 4(STMN4) |
| SOX2 | BM722297 | Homo sapiens | SRY-box 2(SOX2) |
| SOX2 | KM822781 | Homo sapiens | SRY-box 2(SOX2) |
| SOX2 | AW016610 | Homo sapiens | SRY-box 2(SOX2) |
| SOX2 | BC013923 | Homo sapiens | SRY-box 2(SOX2) |
| SOX2 | L07335 | Homo sapiens | SRY-box 2(SOX2) |
| SOX2 | BM668019 | Homo sapiens | SRY-box 2(SOX2) |
| SOX2 | NM_003106 | Homo sapiens | SRY-box 2(SOX2) |
| SOX2 | Z31560 | Homo sapiens | SRY-box 2(SOX2) |
| SOX2 | AK312595 | Homo sapiens | SRY-box 2(SOX2) |
| SOX2 | BF305585 | Homo sapiens | SRY-box 2(SOX2) |
| SOX2 | CN430685 | Homo sapiens | SRY-box 2(SOX2) |
| SOX2 | DA388996 | Homo sapiens | SRY-box 2(SOX2) |
| SOX2 | EU446654 | Homo sapiens | SRY-box 2(SOX2) |
| SOX2 | AW163619 | Homo sapiens | SRY-box 2(SOX2) |
| CDH6 | XM_017008910 | Homo sapiens | cadherin 6(CDH6) |
| CDH6 | AK291290 | Homo sapiens | cadherin 6(CDH6) |
| CDH6 | XM_017008911 | Homo sapiens | cadherin 6(CDH6) |
| CDH6 | DC353791 | Homo sapiens | cadherin 6(CDH6) |
| CDH6 | AL049227 | Homo sapiens | cadherin 6(CDH6) |
| CDH6 | XR_001741972 | Homo sapiens | cadherin 6(CDH6) |
| CDH6 | BC013907 | Homo sapiens | cadherin 6(CDH6) |
| CDH6 | NM_004932 | Homo sapiens | cadherin 6(CDH6) |
| CDH6 | AK024238 | Homo sapiens | cadherin 6(CDH6) |
| CDH6 | XM_011513921 | Homo sapiens | cadherin 6(CDH6) |
| CDH6 | AU130185 | Homo sapiens | cadherin 6(CDH6) |
| CDH6 | BC000019 | Homo sapiens | cadherin 6(CDH6) |
| CDH6 | D31784 | Homo sapiens | cadherin 6(CDH6) |
| EDNRA | AK304451 | Homo sapiens | endothelin receptor type A(EDNRA) |
| EDNRA | S81539 | Homo sapiens | endothelin receptor type A(EDNRA) |
| EDNRA | DA183901 | Homo sapiens | endothelin receptor type A(EDNRA) |
| EDNRA | BQ006584 | Homo sapiens | endothelin receptor type A(EDNRA) |
| EDNRA | NM_001166055 | Homo sapiens | endothelin receptor type A(EDNRA) |
| EDNRA | L06622 | Homo sapiens | endothelin receptor type A(EDNRA) |
| EDNRA | NM_001256283 | Homo sapiens | endothelin receptor type A(EDNRA) |
| EDNRA | DA956937 | Homo sapiens | endothelin receptor type A(EDNRA) |
| EDNRA | S45956 | Homo sapiens | endothelin receptor type A(EDNRA) |
| EDNRA | S67127 | Homo sapiens | endothelin receptor type A(EDNRA) |
| EDNRA | S57498 | Homo sapiens | endothelin receptor type A(EDNRA) |
| EDNRA | X61950 | Homo sapiens | endothelin receptor type A(EDNRA) |
| EDNRA | AK312812 | Homo sapiens | endothelin receptor type A(EDNRA) |
| EDNRA | NM_001957 | Homo sapiens | endothelin receptor type A(EDNRA) |
| EDNRA | DQ892329 | Homo sapiens | endothelin receptor type A(EDNRA) |
| EDNRA | S81545 | Homo sapiens | endothelin receptor type A(EDNRA) |
| EDNRA | BC022511 | Homo sapiens | endothelin receptor type A(EDNRA) |
| EDNRA | DA775295 | Homo sapiens | endothelin receptor type A(EDNRA) |
| EDNRA | S81542 | Homo sapiens | endothelin receptor type A(EDNRA) |
| EDNRA | NR_045958 | Homo sapiens | endothelin receptor type A(EDNRA) |
| EDNRA | AY275462 | Homo sapiens | endothelin receptor type A(EDNRA) |
| EDNRA | CD723797 | Homo sapiens | endothelin receptor type A(EDNRA) |
| EDNRA | AK315931 | Homo sapiens | endothelin receptor type A(EDNRA) |
| EDNRA | DQ895532 | Homo sapiens | endothelin receptor type A(EDNRA) |
| EDNRA | AF014826 | Homo sapiens | endothelin receptor type A(EDNRA) |
| EDNRA | S63938 | Homo sapiens | endothelin receptor type A(EDNRA) |
| EDNRA | D90348 | Homo sapiens | endothelin receptor type A(EDNRA) |
| EDNRB | S75587 | Homo sapiens | endothelin receptor type B(EDNRB) |
| EDNRB | NM_000115 | Homo sapiens | endothelin receptor type B(EDNRB) |
| EDNRB | NM_001122659 | Homo sapiens | endothelin receptor type B(EDNRB) |
| EDNRB | AF114164 | Homo sapiens | endothelin receptor type B(EDNRB) |
| EDNRB | AF114165 | Homo sapiens | endothelin receptor type B(EDNRB) |
| EDNRB | M74921 | Homo sapiens | endothelin receptor type B(EDNRB) |
| EDNRB | L06623 | Homo sapiens | endothelin receptor type B(EDNRB) |
| EDNRB | AB209198 | Homo sapiens | endothelin receptor type B(EDNRB) |
| EDNRB | AY275463 | Homo sapiens | endothelin receptor type B(EDNRB) |
| EDNRB | S44866 | Homo sapiens | endothelin receptor type B(EDNRB) |
| EDNRB | BM557607 | Homo sapiens | endothelin receptor type B(EDNRB) |
| EDNRB | NR_047024 | Homo sapiens | endothelin receptor type B(EDNRB) |
| EDNRB | AF114163 | Homo sapiens | endothelin receptor type B(EDNRB) |
| EDNRB | BG436360 | Homo sapiens | endothelin receptor type B(EDNRB) |
| EDNRB | NM_003991 | Homo sapiens | endothelin receptor type B(EDNRB) |
| EDNRB | D90402 | Homo sapiens | endothelin receptor type B(EDNRB) |
| EDNRB | NM_001201397 | Homo sapiens | endothelin receptor type B(EDNRB) |
| EDNRB | AK290699 | Homo sapiens | endothelin receptor type B(EDNRB) |
| EDNRB | X99250 | Homo sapiens | endothelin receptor type B(EDNRB) |
| EDNRB | S57283 | Homo sapiens | endothelin receptor type B(EDNRB) |
| EDNRB | BC014472 | Homo sapiens | endothelin receptor type B(EDNRB) |
| EDNRB | H28710 | Homo sapiens | endothelin receptor type B(EDNRB) |
| CDH1 | XM_011523488 | Homo sapiens | cadherin 1(CDH1) |
| CDH1 | BC013851 | Homo sapiens | cadherin 1(CDH1) |
| CDH1 | X12790 | Homo sapiens | cadherin 1(CDH1) |
| CDH1 | XM_011523489 | Homo sapiens | cadherin 1(CDH1) |
| CDH1 | BC146662 | Homo sapiens | cadherin 1(CDH1) |
| CDH1 | EU709494 | Homo sapiens | cadherin 1(CDH1) |
| CDH1 | Z13009 | Homo sapiens | cadherin 1(CDH1) |
| CDH1 | AB025106 | Homo sapiens | cadherin 1(CDH1) |
| CDH1 | AB025105 | Homo sapiens | cadherin 1(CDH1) |
| CDH1 | AI890107 | Homo sapiens | cadherin 1(CDH1) |
| CDH1 | AK309703 | Homo sapiens | cadherin 1(CDH1) |
| CDH1 | L08599 | Homo sapiens | cadherin 1(CDH1) |
| CDH1 | BC141838 | Homo sapiens | cadherin 1(CDH1) |
| CDH1 | NM_004360 | Homo sapiens | cadherin 1(CDH1) |
| CDH1 | X52279 | Homo sapiens | cadherin 1(CDH1) |
| CDH1 | AK311198 | Homo sapiens | cadherin 1(CDH1) |
| CDH1 | AK312551 | Homo sapiens | cadherin 1(CDH1) |
| CDH1 | NM_001317184 | Homo sapiens | cadherin 1(CDH1) |
| CDH1 | AK290012 | Homo sapiens | cadherin 1(CDH1) |
| CDH1 | Z18923 | Homo sapiens | cadherin 1(CDH1) |
| CDH1 | AK297913 | Homo sapiens | cadherin 1(CDH1) |
| CDH1 | NM_001317186 | Homo sapiens | cadherin 1(CDH1) |
| CDH1 | NM_001317185 | Homo sapiens | cadherin 1(CDH1) |
| CDH1 | BC144283 | Homo sapiens | cadherin 1(CDH1) |
| MPZ | BC006491 | Homo sapiens | myelin protein zero(MPZ) |
| MPZ | CD172418 | Homo sapiens | myelin protein zero(MPZ) |
| MPZ | D10537 | Homo sapiens | myelin protein zero(MPZ) |
| MPZ | NM_000530 | Homo sapiens | myelin protein zero(MPZ) |
| MPZ | BM663255 | Homo sapiens | myelin protein zero(MPZ) |
| MPZ | XM_017001321 | Homo sapiens | myelin protein zero(MPZ) |
| MPZ | BT006765 | Homo sapiens | myelin protein zero(MPZ) |
| MPZ | CD515400 | Homo sapiens | myelin protein zero(MPZ) |
| MPZ | DB210759 | Homo sapiens | myelin protein zero(MPZ) |
| MPZ | CD515605 | Homo sapiens | myelin protein zero(MPZ) |
| MPZ | EU176378 | Homo sapiens | myelin protein zero(MPZ) |
| MPZ | BF509916 | Homo sapiens | myelin protein zero(MPZ) |
| MPZ | DQ895885 | Homo sapiens | myelin protein zero(MPZ) |
| MPZ | NM_001315491 | Homo sapiens | myelin protein zero(MPZ) |
| MPZ | AK313555 | Homo sapiens | myelin protein zero(MPZ) |
| MPZ | S66705 | Homo sapiens | myelin protein zero(MPZ) |
| NRCAM | BX538010 | Homo sapiens | neuronal cell adhesion molecule(NRCAM) |
| NRCAM | XM_017012239 | Homo sapiens | neuronal cell adhesion molecule(NRCAM) |
| NRCAM | XM_017012238 | Homo sapiens | neuronal cell adhesion molecule(NRCAM) |
| NRCAM | XM_017012237 | Homo sapiens | neuronal cell adhesion molecule(NRCAM) |
| NRCAM | XM_017012236 | Homo sapiens | neuronal cell adhesion molecule(NRCAM) |
| NRCAM | XM_005250385 | Homo sapiens | neuronal cell adhesion molecule(NRCAM) |
| NRCAM | XM_005250383 | Homo sapiens | neuronal cell adhesion molecule(NRCAM) |
| NRCAM | BC114570 | Homo sapiens | neuronal cell adhesion molecule(NRCAM) |
| NRCAM | BC115736 | Homo sapiens | neuronal cell adhesion molecule(NRCAM) |
| NRCAM | AK299870 | Homo sapiens | neuronal cell adhesion molecule(NRCAM) |
| NRCAM | BX954399 | Homo sapiens | neuronal cell adhesion molecule(NRCAM) |
| NRCAM | XM_011516256 | Homo sapiens | neuronal cell adhesion molecule(NRCAM) |
| NRCAM | XM_011516255 | Homo sapiens | neuronal cell adhesion molecule(NRCAM) |
| NRCAM | XM_005250373 | Homo sapiens | neuronal cell adhesion molecule(NRCAM) |
| NRCAM | XM_011516253 | Homo sapiens | neuronal cell adhesion molecule(NRCAM) |
| NRCAM | DA398450 | Homo sapiens | neuronal cell adhesion molecule(NRCAM) |
| NRCAM | NM_001037133 | Homo sapiens | neuronal cell adhesion molecule(NRCAM) |
| NRCAM | AJ001057 | Homo sapiens | neuronal cell adhesion molecule(NRCAM) |
| NRCAM | NM_001037132 | Homo sapiens | neuronal cell adhesion molecule(NRCAM) |
| NRCAM | AJ001054 | Homo sapiens | neuronal cell adhesion molecule(NRCAM) |
| NRCAM | XM_017012259 | Homo sapiens | neuronal cell adhesion molecule(NRCAM) |
| NRCAM | XM_017012258 | Homo sapiens | neuronal cell adhesion molecule(NRCAM) |
| NRCAM | BU735065 | Homo sapiens | neuronal cell adhesion molecule(NRCAM) |
| NRCAM | XM_011516267 | Homo sapiens | neuronal cell adhesion molecule(NRCAM) |
| NRCAM | AY528240 | Homo sapiens | neuronal cell adhesion molecule(NRCAM) |
| NRCAM | XM_011516266 | Homo sapiens | neuronal cell adhesion molecule(NRCAM) |
| NRCAM | XM_011516265 | Homo sapiens | neuronal cell adhesion molecule(NRCAM) |
| NRCAM | XM_011516262 | Homo sapiens | neuronal cell adhesion molecule(NRCAM) |
| NRCAM | XM_011516261 | Homo sapiens | neuronal cell adhesion molecule(NRCAM) |
| NRCAM | NM_005010 | Homo sapiens | neuronal cell adhesion molecule(NRCAM) |
| NRCAM | AK127035 | Homo sapiens | neuronal cell adhesion molecule(NRCAM) |
| NRCAM | AK092330 | Homo sapiens | neuronal cell adhesion molecule(NRCAM) |
| NRCAM | NM_001193583 | Homo sapiens | neuronal cell adhesion molecule(NRCAM) |
| NRCAM | NM_001193584 | Homo sapiens | neuronal cell adhesion molecule(NRCAM) |
| NRCAM | XM_011516259 | Homo sapiens | neuronal cell adhesion molecule(NRCAM) |
| NRCAM | XM_011516258 | Homo sapiens | neuronal cell adhesion molecule(NRCAM) |
| NRCAM | NM_001193582 | Homo sapiens | neuronal cell adhesion molecule(NRCAM) |
| NRCAM | XM_011516257 | Homo sapiens | neuronal cell adhesion molecule(NRCAM) |
| NRCAM | XM_006716007 | Homo sapiens | neuronal cell adhesion molecule(NRCAM) |
| NRCAM | XM_017012253 | Homo sapiens | neuronal cell adhesion molecule(NRCAM) |
| NRCAM | XM_017012252 | Homo sapiens | neuronal cell adhesion molecule(NRCAM) |
| NRCAM | XM_017012251 | Homo sapiens | neuronal cell adhesion molecule(NRCAM) |
| NRCAM | XM_017012250 | Homo sapiens | neuronal cell adhesion molecule(NRCAM) |
| NRCAM | XM_006716003 | Homo sapiens | neuronal cell adhesion molecule(NRCAM) |
| NRCAM | XM_017012257 | Homo sapiens | neuronal cell adhesion molecule(NRCAM) |
| NRCAM | XM_017012256 | Homo sapiens | neuronal cell adhesion molecule(NRCAM) |
| NRCAM | XM_017012255 | Homo sapiens | neuronal cell adhesion molecule(NRCAM) |
| NRCAM | XM_017012254 | Homo sapiens | neuronal cell adhesion molecule(NRCAM) |
| NRCAM | XM_011516270 | Homo sapiens | neuronal cell adhesion molecule(NRCAM) |
| NRCAM | XM_017012249 | Homo sapiens | neuronal cell adhesion molecule(NRCAM) |
| NRCAM | XM_017012248 | Homo sapiens | neuronal cell adhesion molecule(NRCAM) |
| NRCAM | XM_017012247 | Homo sapiens | neuronal cell adhesion molecule(NRCAM) |
| NRCAM | AK294195 | Homo sapiens | neuronal cell adhesion molecule(NRCAM) |
| NRCAM | XM_011516271 | Homo sapiens | neuronal cell adhesion molecule(NRCAM) |
| NRCAM | AI031622 | Homo sapiens | neuronal cell adhesion molecule(NRCAM) |
| NRCAM | BC098401 | Homo sapiens | neuronal cell adhesion molecule(NRCAM) |
| NRCAM | AB002341 | Homo sapiens | neuronal cell adhesion molecule(NRCAM) |
| NRCAM | DA292156 | Homo sapiens | neuronal cell adhesion molecule(NRCAM) |
| NRCAM | XM_011516269 | Homo sapiens | neuronal cell adhesion molecule(NRCAM) |
| NRCAM | XM_011516268 | Homo sapiens | neuronal cell adhesion molecule(NRCAM) |
| NRCAM | XM_017012242 | Homo sapiens | neuronal cell adhesion molecule(NRCAM) |
| NRCAM | XM_017012241 | Homo sapiens | neuronal cell adhesion molecule(NRCAM) |
| NRCAM | XM_017012240 | Homo sapiens | neuronal cell adhesion molecule(NRCAM) |
| NRCAM | XM_006716014 | Homo sapiens | neuronal cell adhesion molecule(NRCAM) |
| NRCAM | XM_017012246 | Homo sapiens | neuronal cell adhesion molecule(NRCAM) |
| NRCAM | XM_017012245 | Homo sapiens | neuronal cell adhesion molecule(NRCAM) |
| NRCAM | XM_006716012 | Homo sapiens | neuronal cell adhesion molecule(NRCAM) |
| NRCAM | XM_017012244 | Homo sapiens | neuronal cell adhesion molecule(NRCAM) |
| NRCAM | XM_017012243 | Homo sapiens | neuronal cell adhesion molecule(NRCAM) |
| NCKAP5 | NM_207481 | Homo sapiens | NCK associated protein 5(NCKAP5) |
| NCKAP5 | XM_005263659 | Homo sapiens | NCK associated protein 5(NCKAP5) |
| NCKAP5 | NM_207363 | Homo sapiens | NCK associated protein 5(NCKAP5) |
| NCKAP5 | AK057980 | Homo sapiens | NCK associated protein 5(NCKAP5) |
| NCKAP5 | CA438539 | Homo sapiens | NCK associated protein 5(NCKAP5) |
| NCKAP5 | AB005217 | Homo sapiens | NCK associated protein 5(NCKAP5) |
| NCKAP5 | XM_011511097 | Homo sapiens | NCK associated protein 5(NCKAP5) |
| NCKAP5 | XM_017003980 | Homo sapiens | NCK associated protein 5(NCKAP5) |
| NCKAP5 | XM_011511103 | Homo sapiens | NCK associated protein 5(NCKAP5) |
| NCKAP5 | XM_011511102 | Homo sapiens | NCK associated protein 5(NCKAP5) |
| NCKAP5 | XM_011511105 | Homo sapiens | NCK associated protein 5(NCKAP5) |
| NCKAP5 | XM_011511104 | Homo sapiens | NCK associated protein 5(NCKAP5) |
| NCKAP5 | XM_011511101 | Homo sapiens | NCK associated protein 5(NCKAP5) |
| NCKAP5 | XM_011511100 | Homo sapiens | NCK associated protein 5(NCKAP5) |
| NCKAP5 | BC172401 | Homo sapiens | NCK associated protein 5(NCKAP5) |
| NCKAP5 | AY946008 | Homo sapiens | NCK associated protein 5(NCKAP5) |
| NCKAP5 | AY946007 | Homo sapiens | NCK associated protein 5(NCKAP5) |
| NCKAP5 | AK124659 | Homo sapiens | NCK associated protein 5(NCKAP5) |
| NCKAP5 | XM_017003974 | Homo sapiens | NCK associated protein 5(NCKAP5) |
| NCKAP5 | AK092189 | Homo sapiens | NCK associated protein 5(NCKAP5) |
| NCKAP5 | XM_017003979 | Homo sapiens | NCK associated protein 5(NCKAP5) |
| NCKAP5 | BC110831 | Homo sapiens | NCK associated protein 5(NCKAP5) |
| NCKAP5 | XM_011511099 | Homo sapiens | NCK associated protein 5(NCKAP5) |
| NCKAP5 | XM_017003976 | Homo sapiens | NCK associated protein 5(NCKAP5) |
| NCKAP5 | XM_011511098 | Homo sapiens | NCK associated protein 5(NCKAP5) |
| NCKAP5 | XM_017003975 | Homo sapiens | NCK associated protein 5(NCKAP5) |
| NCKAP5 | XM_017003978 | Homo sapiens | NCK associated protein 5(NCKAP5) |
| NCKAP5 | XM_017003977 | Homo sapiens | NCK associated protein 5(NCKAP5) |
| NCKAP5 | XM_005263660 | Homo sapiens | NCK associated protein 5(NCKAP5) |
| WLS | XM_017002390 | Homo sapiens | wntless Wnt ligand secretion mediator(WLS) |
| WLS | AB097018 | Homo sapiens | wntless Wnt ligand secretion mediator(WLS) |
| WLS | AW137622 | Homo sapiens | wntless Wnt ligand secretion mediator(WLS) |
| WLS | BC007211 | Homo sapiens | wntless Wnt ligand secretion mediator(WLS) |
| WLS | AK074583 | Homo sapiens | wntless Wnt ligand secretion mediator(WLS) |
| WLS | BC137113 | Homo sapiens | wntless Wnt ligand secretion mediator(WLS) |
| WLS | AK026744 | Homo sapiens | wntless Wnt ligand secretion mediator(WLS) |
| WLS | AY359035 | Homo sapiens | wntless Wnt ligand secretion mediator(WLS) |
| WLS | CA438784 | Homo sapiens | wntless Wnt ligand secretion mediator(WLS) |
| WLS | BC137109 | Homo sapiens | wntless Wnt ligand secretion mediator(WLS) |
| WLS | AK074984 | Homo sapiens | wntless Wnt ligand secretion mediator(WLS) |
| WLS | DB478499 | Homo sapiens | wntless Wnt ligand secretion mediator(WLS) |
| WLS | BC110826 | Homo sapiens | wntless Wnt ligand secretion mediator(WLS) |
| WLS | BX648748 | Homo sapiens | wntless Wnt ligand secretion mediator(WLS) |
| WLS | DA753300 | Homo sapiens | wntless Wnt ligand secretion mediator(WLS) |
| WLS | AK309779 | Homo sapiens | wntless Wnt ligand secretion mediator(WLS) |
| WLS | BX537492 | Homo sapiens | wntless Wnt ligand secretion mediator(WLS) |
| WLS | NM_024911 | Homo sapiens | wntless Wnt ligand secretion mediator(WLS) |
| WLS | NM_001002292 | Homo sapiens | wntless Wnt ligand secretion mediator(WLS) |
| WLS | BX538320 | Homo sapiens | wntless Wnt ligand secretion mediator(WLS) |
| WLS | AK301613 | Homo sapiens | wntless Wnt ligand secretion mediator(WLS) |
| WLS | AI217373 | Homo sapiens | wntless Wnt ligand secretion mediator(WLS) |
| WLS | NM_001193334 | Homo sapiens | wntless Wnt ligand secretion mediator(WLS) |
| WLS | DQ323735 | Homo sapiens | wntless Wnt ligand secretion mediator(WLS) |
| WLS | BG701224 | Homo sapiens | wntless Wnt ligand secretion mediator(WLS) |
| WLS | XM_011542191 | Homo sapiens | wntless Wnt ligand secretion mediator(WLS) |
| WLS | XM_011542192 | Homo sapiens | wntless Wnt ligand secretion mediator(WLS) |
| PDGFRB | J03278 | Homo sapiens | platelet derived growth factor receptor beta(PDGFRB) |
| PDGFRB | AK293093 | Homo sapiens | platelet derived growth factor receptor beta(PDGFRB) |
| PDGFRB | NM_002609 | Homo sapiens | platelet derived growth factor receptor beta(PDGFRB) |
| PDGFRB | AB209657 | Homo sapiens | platelet derived growth factor receptor beta(PDGFRB) |
| PDGFRB | EU826595 | Homo sapiens | platelet derived growth factor receptor beta(PDGFRB) |
| PDGFRB | M30493 | Homo sapiens | platelet derived growth factor receptor beta(PDGFRB) |
| PDGFRB | XM_005268464 | Homo sapiens | platelet derived growth factor receptor beta(PDGFRB) |
| PDGFRB | XM_011537658 | Homo sapiens | platelet derived growth factor receptor beta(PDGFRB) |
| PDGFRB | M21616 | Homo sapiens | platelet derived growth factor receptor beta(PDGFRB) |
| PDGFRB | XM_011537659 | Homo sapiens | platelet derived growth factor receptor beta(PDGFRB) |
| PDGFRB | AI346188 | Homo sapiens | platelet derived growth factor receptor beta(PDGFRB) |
| PDGFRB | EU176549 | Homo sapiens | platelet derived growth factor receptor beta(PDGFRB) |
| PDGFRB | BC032224 | Homo sapiens | platelet derived growth factor receptor beta(PDGFRB) |
| PDGFRB | DQ892124 | Homo sapiens | platelet derived growth factor receptor beta(PDGFRB) |
| PDGFRB | CN268096 | Homo sapiens | platelet derived growth factor receptor beta(PDGFRB) |
| PDGFRA | XM_006714041 | Homo sapiens | platelet derived growth factor receptor alpha(PDGFRA) |
| PDGFRA | BC063414 | Homo sapiens | platelet derived growth factor receptor alpha(PDGFRA) |
| PDGFRA | AK308353 | Homo sapiens | platelet derived growth factor receptor alpha(PDGFRA) |
| PDGFRA | XM_005265743 | Homo sapiens | platelet derived growth factor receptor alpha(PDGFRA) |
| PDGFRA | XM_017008282 | Homo sapiens | platelet derived growth factor receptor alpha(PDGFRA) |
| PDGFRA | NM_006206 | Homo sapiens | platelet derived growth factor receptor alpha(PDGFRA) |
| PDGFRA | AA599881 | Homo sapiens | platelet derived growth factor receptor alpha(PDGFRA) |
| PDGFRA | M30494 | Homo sapiens | platelet derived growth factor receptor alpha(PDGFRA) |
| PDGFRA | DA678599 | Homo sapiens | platelet derived growth factor receptor alpha(PDGFRA) |
| PDGFRA | X76079 | Homo sapiens | platelet derived growth factor receptor alpha(PDGFRA) |
| PDGFRA | AV689272 | Homo sapiens | platelet derived growth factor receptor alpha(PDGFRA) |
| PDGFRA | XM_011534385 | Homo sapiens | platelet derived growth factor receptor alpha(PDGFRA) |
| PDGFRA | M22734 | Homo sapiens | platelet derived growth factor receptor alpha(PDGFRA) |
| PDGFRA | XM_006714039 | Homo sapiens | platelet derived growth factor receptor alpha(PDGFRA) |
| PDGFRA | XM_017008281 | Homo sapiens | platelet derived growth factor receptor alpha(PDGFRA) |
| PDGFRA | L25829 | Homo sapiens | platelet derived growth factor receptor alpha(PDGFRA) |
| PDGFRA | XM_017008280 | Homo sapiens | platelet derived growth factor receptor alpha(PDGFRA) |
| PDGFRA | AK316578 | Homo sapiens | platelet derived growth factor receptor alpha(PDGFRA) |
| PDGFRA | M21574 | Homo sapiens | platelet derived growth factor receptor alpha(PDGFRA) |
| PDGFRA | AK311006 | Homo sapiens | platelet derived growth factor receptor alpha(PDGFRA) |
| PDGFRA | AA625689 | Homo sapiens | platelet derived growth factor receptor alpha(PDGFRA) |
| PDGFRA | BC015186 | Homo sapiens | platelet derived growth factor receptor alpha(PDGFRA) |
| MEF2C | EU446634 | Homo sapiens | myocyte enhancer factor 2C(MEF2C) |
| MEF2C | AK307883 | Homo sapiens | myocyte enhancer factor 2C(MEF2C) |
| MEF2C | XM_011543400 | Homo sapiens | myocyte enhancer factor 2C(MEF2C) |
| MEF2C | NM_001308002 | Homo sapiens | myocyte enhancer factor 2C(MEF2C) |
| MEF2C | XM_011543401 | Homo sapiens | myocyte enhancer factor 2C(MEF2C) |
| MEF2C | BC152784 | Homo sapiens | myocyte enhancer factor 2C(MEF2C) |
| MEF2C | EU832832 | Homo sapiens | myocyte enhancer factor 2C(MEF2C) |
| MEF2C | GQ129219 | Homo sapiens | myocyte enhancer factor 2C(MEF2C) |
| MEF2C | XM_017009481 | Homo sapiens | myocyte enhancer factor 2C(MEF2C) |
| MEF2C | AW191949 | Homo sapiens | myocyte enhancer factor 2C(MEF2C) |
| MEF2C | FM163484 | Homo sapiens | myocyte enhancer factor 2C(MEF2C) |
| MEF2C | XM_017009482 | Homo sapiens | myocyte enhancer factor 2C(MEF2C) |
| MEF2C | XM_017009483 | Homo sapiens | myocyte enhancer factor 2C(MEF2C) |
| MEF2C | FM180475 | Homo sapiens | myocyte enhancer factor 2C(MEF2C) |
| MEF2C | BP231922 | Homo sapiens | myocyte enhancer factor 2C(MEF2C) |
| MEF2C | AK312472 | Homo sapiens | myocyte enhancer factor 2C(MEF2C) |
| MEF2C | XM_006714625 | Homo sapiens | myocyte enhancer factor 2C(MEF2C) |
| MEF2C | XM_005248511 | Homo sapiens | myocyte enhancer factor 2C(MEF2C) |
| MEF2C | DA516520 | Homo sapiens | myocyte enhancer factor 2C(MEF2C) |
| MEF2C | BC156603 | Homo sapiens | myocyte enhancer factor 2C(MEF2C) |
| MEF2C | S57212 | Homo sapiens | myocyte enhancer factor 2C(MEF2C) |
| MEF2C | AL833274 | Homo sapiens | myocyte enhancer factor 2C(MEF2C) |
| MEF2C | DC318557 | Homo sapiens | myocyte enhancer factor 2C(MEF2C) |
| MEF2C | DC377336 | Homo sapiens | myocyte enhancer factor 2C(MEF2C) |
| MEF2C | DC377710 | Homo sapiens | myocyte enhancer factor 2C(MEF2C) |
| MEF2C | NM_001193349 | Homo sapiens | myocyte enhancer factor 2C(MEF2C) |
| MEF2C | NM_001193347 | Homo sapiens | myocyte enhancer factor 2C(MEF2C) |
| MEF2C | NM_001193348 | Homo sapiens | myocyte enhancer factor 2C(MEF2C) |
| MEF2C | XM_017009478 | Homo sapiens | myocyte enhancer factor 2C(MEF2C) |
| MEF2C | XM_017009479 | Homo sapiens | myocyte enhancer factor 2C(MEF2C) |
| MEF2C | XM_011543396 | Homo sapiens | myocyte enhancer factor 2C(MEF2C) |
| MEF2C | XM_011543397 | Homo sapiens | myocyte enhancer factor 2C(MEF2C) |
| MEF2C | GQ129221 | Homo sapiens | myocyte enhancer factor 2C(MEF2C) |
| MEF2C | BC026341 | Homo sapiens | myocyte enhancer factor 2C(MEF2C) |
| MEF2C | XM_017009475 | Homo sapiens | myocyte enhancer factor 2C(MEF2C) |
| MEF2C | XM_006714619 | Homo sapiens | myocyte enhancer factor 2C(MEF2C) |
| MEF2C | XM_017009476 | Homo sapiens | myocyte enhancer factor 2C(MEF2C) |
| MEF2C | L08895 | Homo sapiens | myocyte enhancer factor 2C(MEF2C) |
| MEF2C | XM_017009477 | Homo sapiens | myocyte enhancer factor 2C(MEF2C) |
| MEF2C | NM_001193350 | Homo sapiens | myocyte enhancer factor 2C(MEF2C) |
| MEF2C | NM_002397 | Homo sapiens | myocyte enhancer factor 2C(MEF2C) |
| MEF2C | XM_017009480 | Homo sapiens | myocyte enhancer factor 2C(MEF2C) |
| MEF2C | GQ129392 | Homo sapiens | myocyte enhancer factor 2C(MEF2C) |
| MEF2C | GQ129393 | Homo sapiens | myocyte enhancer factor 2C(MEF2C) |
| MEF2C | NM_001131005 | Homo sapiens | myocyte enhancer factor 2C(MEF2C) |
| MEF2C | AL833268 | Homo sapiens | myocyte enhancer factor 2C(MEF2C) |
| MEF2C | DA494302 | Homo sapiens | myocyte enhancer factor 2C(MEF2C) |
| RBFOX3 | NM_001039904 | Homo sapiens | RNA binding protein, fox-1 homolog 3(RBFOX3) |
| RBFOX3 | NM_001082575 | Homo sapiens | RNA binding protein, fox-1 homolog 3(RBFOX3) |
| RBFOX3 | XM_011524365 | Homo sapiens | RNA binding protein, fox-1 homolog 3(RBFOX3) |
| RBFOX3 | AK054893 | Homo sapiens | RNA binding protein, fox-1 homolog 3(RBFOX3) |
| RBFOX3 | XM_011524367 | Homo sapiens | RNA binding protein, fox-1 homolog 3(RBFOX3) |
| RBFOX3 | XM_011524366 | Homo sapiens | RNA binding protein, fox-1 homolog 3(RBFOX3) |
| RBFOX3 | BC140939 | Homo sapiens | RNA binding protein, fox-1 homolog 3(RBFOX3) |
| RBFOX3 | LK938159 | Homo sapiens | RNA binding protein, fox-1 homolog 3(RBFOX3) |
| RBFOX3 | BM714144 | Homo sapiens | RNA binding protein, fox-1 homolog 3(RBFOX3) |
| RBFOX3 | BU741507 | Homo sapiens | RNA binding protein, fox-1 homolog 3(RBFOX3) |
| RBFOX3 | XM_017024208 | Homo sapiens | RNA binding protein, fox-1 homolog 3(RBFOX3) |
| RBFOX3 | AK293617 | Homo sapiens | RNA binding protein, fox-1 homolog 3(RBFOX3) |
| RBFOX3 | XM_017024209 | Homo sapiens | RNA binding protein, fox-1 homolog 3(RBFOX3) |
| RBFOX3 | AK124644 | Homo sapiens | RNA binding protein, fox-1 homolog 3(RBFOX3) |
| RBFOX3 | AK126788 | Homo sapiens | RNA binding protein, fox-1 homolog 3(RBFOX3) |
| RBFOX3 | NM_001025448 | Homo sapiens | RNA binding protein, fox-1 homolog 3(RBFOX3) |
| RBFOX3 | BC093713 | Homo sapiens | RNA binding protein, fox-1 homolog 3(RBFOX3) |
| RBFOX3 | XM_011524359 | Homo sapiens | RNA binding protein, fox-1 homolog 3(RBFOX3) |
| RBFOX3 | BX452143 | Homo sapiens | RNA binding protein, fox-1 homolog 3(RBFOX3) |
| RBFOX3 | XM_017024210 | Homo sapiens | RNA binding protein, fox-1 homolog 3(RBFOX3) |
| RBFOX3 | XM_017024211 | Homo sapiens | RNA binding protein, fox-1 homolog 3(RBFOX3) |
| RBFOX3 | AK293905 | Homo sapiens | RNA binding protein, fox-1 homolog 3(RBFOX3) |
| RBFOX3 | DN990270 | Homo sapiens | RNA binding protein, fox-1 homolog 3(RBFOX3) |
| RBFOX3 | XM_011524360 | Homo sapiens | RNA binding protein, fox-1 homolog 3(RBFOX3) |
| RBFOX3 | AK128131 | Homo sapiens | RNA binding protein, fox-1 homolog 3(RBFOX3) |
| RBFOX3 | XM_011524363 | Homo sapiens | RNA binding protein, fox-1 homolog 3(RBFOX3) |
| RBFOX3 | XM_011524362 | Homo sapiens | RNA binding protein, fox-1 homolog 3(RBFOX3) |
| LIMCH1 | AK299835 | Homo sapiens | LIM and calponin homology domains 1(LIMCH1) |
| LIMCH1 | NM_001330674 | Homo sapiens | LIM and calponin homology domains 1(LIMCH1) |
| LIMCH1 | XM_005248060 | Homo sapiens | LIM and calponin homology domains 1(LIMCH1) |
| LIMCH1 | NM_001330672 | Homo sapiens | LIM and calponin homology domains 1(LIMCH1) |
| LIMCH1 | NM_001330793 | Homo sapiens | LIM and calponin homology domains 1(LIMCH1) |
| LIMCH1 | AM393081 | Homo sapiens | LIM and calponin homology domains 1(LIMCH1) |
| LIMCH1 | NM_001330791 | Homo sapiens | LIM and calponin homology domains 1(LIMCH1) |
| LIMCH1 | NM_001330792 | Homo sapiens | LIM and calponin homology domains 1(LIMCH1) |
| LIMCH1 | NM_001112718 | Homo sapiens | LIM and calponin homology domains 1(LIMCH1) |
| LIMCH1 | NM_001112717 | Homo sapiens | LIM and calponin homology domains 1(LIMCH1) |
| LIMCH1 | AK309846 | Homo sapiens | LIM and calponin homology domains 1(LIMCH1) |
| LIMCH1 | NM_001112719 | Homo sapiens | LIM and calponin homology domains 1(LIMCH1) |
| LIMCH1 | BC029735 | Homo sapiens | LIM and calponin homology domains 1(LIMCH1) |
| LIMCH1 | XM_011513645 | Homo sapiens | LIM and calponin homology domains 1(LIMCH1) |
| LIMCH1 | XM_011513646 | Homo sapiens | LIM and calponin homology domains 1(LIMCH1) |
| LIMCH1 | XM_011513643 | Homo sapiens | LIM and calponin homology domains 1(LIMCH1) |
| LIMCH1 | XM_011513644 | Homo sapiens | LIM and calponin homology domains 1(LIMCH1) |
| LIMCH1 | XM_005248058 | Homo sapiens | LIM and calponin homology domains 1(LIMCH1) |
| LIMCH1 | CR749205 | Homo sapiens | LIM and calponin homology domains 1(LIMCH1) |
| LIMCH1 | XM_011513642 | Homo sapiens | LIM and calponin homology domains 1(LIMCH1) |
| LIMCH1 | XM_006713996 | Homo sapiens | LIM and calponin homology domains 1(LIMCH1) |
| LIMCH1 | AK295836 | Homo sapiens | LIM and calponin homology domains 1(LIMCH1) |
| LIMCH1 | NM_001330790 | Homo sapiens | LIM and calponin homology domains 1(LIMCH1) |
| LIMCH1 | XM_005248057 | Homo sapiens | LIM and calponin homology domains 1(LIMCH1) |
| LIMCH1 | XM_011513649 | Homo sapiens | LIM and calponin homology domains 1(LIMCH1) |
| LIMCH1 | CR936664 | Homo sapiens | LIM and calponin homology domains 1(LIMCH1) |
| LIMCH1 | CR936661 | Homo sapiens | LIM and calponin homology domains 1(LIMCH1) |
| LIMCH1 | XM_011513648 | Homo sapiens | LIM and calponin homology domains 1(LIMCH1) |
| LIMCH1 | NM_001330786 | Homo sapiens | LIM and calponin homology domains 1(LIMCH1) |
| LIMCH1 | AK295784 | Homo sapiens | LIM and calponin homology domains 1(LIMCH1) |
| LIMCH1 | NM_001330787 | Homo sapiens | LIM and calponin homology domains 1(LIMCH1) |
| LIMCH1 | NM_001330784 | Homo sapiens | LIM and calponin homology domains 1(LIMCH1) |
| LIMCH1 | NM_001330982 | Homo sapiens | LIM and calponin homology domains 1(LIMCH1) |
| LIMCH1 | NM_001330983 | Homo sapiens | LIM and calponin homology domains 1(LIMCH1) |
| LIMCH1 | AL117572 | Homo sapiens | LIM and calponin homology domains 1(LIMCH1) |
| LIMCH1 | NM_001330788 | Homo sapiens | LIM and calponin homology domains 1(LIMCH1) |
| LIMCH1 | NM_001330789 | Homo sapiens | LIM and calponin homology domains 1(LIMCH1) |
| LIMCH1 | BC095394 | Homo sapiens | LIM and calponin homology domains 1(LIMCH1) |
| LIMCH1 | XM_011513656 | Homo sapiens | LIM and calponin homology domains 1(LIMCH1) |
| LIMCH1 | AL831962 | Homo sapiens | LIM and calponin homology domains 1(LIMCH1) |
| LIMCH1 | XM_011513657 | Homo sapiens | LIM and calponin homology domains 1(LIMCH1) |
| LIMCH1 | XM_011513654 | Homo sapiens | LIM and calponin homology domains 1(LIMCH1) |
| LIMCH1 | XM_011513655 | Homo sapiens | LIM and calponin homology domains 1(LIMCH1) |
| LIMCH1 | BC053639 | Homo sapiens | LIM and calponin homology domains 1(LIMCH1) |
| LIMCH1 | CR933645 | Homo sapiens | LIM and calponin homology domains 1(LIMCH1) |
| LIMCH1 | DA786971 | Homo sapiens | LIM and calponin homology domains 1(LIMCH1) |
| LIMCH1 | XM_011513653 | Homo sapiens | LIM and calponin homology domains 1(LIMCH1) |
| LIMCH1 | AB029025 | Homo sapiens | LIM and calponin homology domains 1(LIMCH1) |
| LIMCH1 | XM_011513651 | Homo sapiens | LIM and calponin homology domains 1(LIMCH1) |
| LIMCH1 | AK311596 | Homo sapiens | LIM and calponin homology domains 1(LIMCH1) |
| LIMCH1 | XM_005248067 | Homo sapiens | LIM and calponin homology domains 1(LIMCH1) |
| LIMCH1 | AK125004 | Homo sapiens | LIM and calponin homology domains 1(LIMCH1) |
| LIMCH1 | AK294774 | Homo sapiens | LIM and calponin homology domains 1(LIMCH1) |
| LIMCH1 | XM_005248061 | Homo sapiens | LIM and calponin homology domains 1(LIMCH1) |
| LIMCH1 | XM_011513658 | Homo sapiens | LIM and calponin homology domains 1(LIMCH1) |
| LIMCH1 | NM_001289122 | Homo sapiens | LIM and calponin homology domains 1(LIMCH1) |
| LIMCH1 | AK027231 | Homo sapiens | LIM and calponin homology domains 1(LIMCH1) |
| LIMCH1 | AK299297 | Homo sapiens | LIM and calponin homology domains 1(LIMCH1) |
| LIMCH1 | XM_017007901 | Homo sapiens | LIM and calponin homology domains 1(LIMCH1) |
| LIMCH1 | XM_017007900 | Homo sapiens | LIM and calponin homology domains 1(LIMCH1) |
| LIMCH1 | AK302674 | Homo sapiens | LIM and calponin homology domains 1(LIMCH1) |
| LIMCH1 | BC068200 | Homo sapiens | LIM and calponin homology domains 1(LIMCH1) |
| LIMCH1 | CR936601 | Homo sapiens | LIM and calponin homology domains 1(LIMCH1) |
| LIMCH1 | XM_017007905 | Homo sapiens | LIM and calponin homology domains 1(LIMCH1) |
| LIMCH1 | XM_017007904 | Homo sapiens | LIM and calponin homology domains 1(LIMCH1) |
| LIMCH1 | XM_017007903 | Homo sapiens | LIM and calponin homology domains 1(LIMCH1) |
| LIMCH1 | XM_017007902 | Homo sapiens | LIM and calponin homology domains 1(LIMCH1) |
| LIMCH1 | NM_001289124 | Homo sapiens | LIM and calponin homology domains 1(LIMCH1) |
| LIMCH1 | XM_005248072 | Homo sapiens | LIM and calponin homology domains 1(LIMCH1) |
| LIMCH1 | XM_005248074 | Homo sapiens | LIM and calponin homology domains 1(LIMCH1) |
| LIMCH1 | XM_005248075 | Homo sapiens | LIM and calponin homology domains 1(LIMCH1) |
| LIMCH1 | NM_001112720 | Homo sapiens | LIM and calponin homology domains 1(LIMCH1) |
| LIMCH1 | XM_017007899 | Homo sapiens | LIM and calponin homology domains 1(LIMCH1) |
| LIMCH1 | DC325132 | Homo sapiens | LIM and calponin homology domains 1(LIMCH1) |
| LIMCH1 | XM_017007898 | Homo sapiens | LIM and calponin homology domains 1(LIMCH1) |
| LIMCH1 | XM_017007897 | Homo sapiens | LIM and calponin homology domains 1(LIMCH1) |
| LIMCH1 | XM_017007896 | Homo sapiens | LIM and calponin homology domains 1(LIMCH1) |
| LIMCH1 | XM_017007895 | Homo sapiens | LIM and calponin homology domains 1(LIMCH1) |
| LIMCH1 | XM_017007894 | Homo sapiens | LIM and calponin homology domains 1(LIMCH1) |
| LIMCH1 | AK026815 | Homo sapiens | LIM and calponin homology domains 1(LIMCH1) |
| LIMCH1 | BX537916 | Homo sapiens | LIM and calponin homology domains 1(LIMCH1) |
| LIMCH1 | NM_014988 | Homo sapiens | LIM and calponin homology domains 1(LIMCH1) |
| LIMCH1 | CR936610 | Homo sapiens | LIM and calponin homology domains 1(LIMCH1) |
| LIMCH1 | CR936658 | Homo sapiens | LIM and calponin homology domains 1(LIMCH1) |
| LIMCH1 | BC023546 | Homo sapiens | LIM and calponin homology domains 1(LIMCH1) |
| LIMCH1 | BX640692 | Homo sapiens | LIM and calponin homology domains 1(LIMCH1) |
| LIMCH1 | AK298915 | Homo sapiens | LIM and calponin homology domains 1(LIMCH1) |
| LIMCH1 | AM392601 | Homo sapiens | LIM and calponin homology domains 1(LIMCH1) |
| FNDC3B | AK027052 | Homo sapiens | fibronectin type III domain containing 3B(FNDC3B) |
| FNDC3B | NM_022763 | Homo sapiens | fibronectin type III domain containing 3B(FNDC3B) |
| FNDC3B | AF543840 | Homo sapiens | fibronectin type III domain containing 3B(FNDC3B) |
| FNDC3B | AK075220 | Homo sapiens | fibronectin type III domain containing 3B(FNDC3B) |
| FNDC3B | NM_001135095 | Homo sapiens | fibronectin type III domain containing 3B(FNDC3B) |
| FNDC3B | XM_017007064 | Homo sapiens | fibronectin type III domain containing 3B(FNDC3B) |
| FNDC3B | BX648340 | Homo sapiens | fibronectin type III domain containing 3B(FNDC3B) |
| FNDC3B | XM_017007063 | Homo sapiens | fibronectin type III domain containing 3B(FNDC3B) |
| FNDC3B | AI417065 | Homo sapiens | fibronectin type III domain containing 3B(FNDC3B) |
| FNDC3B | AK223599 | Homo sapiens | fibronectin type III domain containing 3B(FNDC3B) |
| FNDC3B | XM_017007062 | Homo sapiens | fibronectin type III domain containing 3B(FNDC3B) |
| FNDC3B | AK127826 | Homo sapiens | fibronectin type III domain containing 3B(FNDC3B) |
| FNDC3B | BC012204 | Homo sapiens | fibronectin type III domain containing 3B(FNDC3B) |
| FNDC3B | AB098597 | Homo sapiens | fibronectin type III domain containing 3B(FNDC3B) |
| FNDC3B | AL157482 | Homo sapiens | fibronectin type III domain containing 3B(FNDC3B) |
| FNDC3B | BC026005 | Homo sapiens | fibronectin type III domain containing 3B(FNDC3B) |
| FNDC3B | AY358367 | Homo sapiens | fibronectin type III domain containing 3B(FNDC3B) |
| FNDC3B | AY358146 | Homo sapiens | fibronectin type III domain containing 3B(FNDC3B) |
| FNDC3B | AK092465 | Homo sapiens | fibronectin type III domain containing 3B(FNDC3B) |
| FNDC3B | BC033635 | Homo sapiens | fibronectin type III domain containing 3B(FNDC3B) |
| FNDC3B | AK314478 | Homo sapiens | fibronectin type III domain containing 3B(FNDC3B) |
| FNDC3B | BC039297 | Homo sapiens | fibronectin type III domain containing 3B(FNDC3B) |
| FNDC3B | BX648415 | Homo sapiens | fibronectin type III domain containing 3B(FNDC3B) |
| DYRK1A | AB015284 | Homo sapiens | dual specificity tyrosine phosphorylation regulated kinase 1A(DYRK1A) |
| DYRK1A | NM_101395 | Homo sapiens | dual specificity tyrosine phosphorylation regulated kinase 1A(DYRK1A) |
| DYRK1A | XM_017028284 | Homo sapiens | dual specificity tyrosine phosphorylation regulated kinase 1A(DYRK1A) |
| DYRK1A | AB015283 | Homo sapiens | dual specificity tyrosine phosphorylation regulated kinase 1A(DYRK1A) |
| DYRK1A | XM_017028285 | Homo sapiens | dual specificity tyrosine phosphorylation regulated kinase 1A(DYRK1A) |
| DYRK1A | AB015282 | Homo sapiens | dual specificity tyrosine phosphorylation regulated kinase 1A(DYRK1A) |
| DYRK1A | XM_017028286 | Homo sapiens | dual specificity tyrosine phosphorylation regulated kinase 1A(DYRK1A) |
| DYRK1A | BC045802 | Homo sapiens | dual specificity tyrosine phosphorylation regulated kinase 1A(DYRK1A) |
| DYRK1A | U58496 | Homo sapiens | dual specificity tyrosine phosphorylation regulated kinase 1A(DYRK1A) |
| DYRK1A | NM_130438 | Homo sapiens | dual specificity tyrosine phosphorylation regulated kinase 1A(DYRK1A) |
| DYRK1A | D85759 | Homo sapiens | dual specificity tyrosine phosphorylation regulated kinase 1A(DYRK1A) |
| DYRK1A | NM_130437 | Homo sapiens | dual specificity tyrosine phosphorylation regulated kinase 1A(DYRK1A) |
| DYRK1A | NM_130436 | Homo sapiens | dual specificity tyrosine phosphorylation regulated kinase 1A(DYRK1A) |
| DYRK1A | U52373 | Homo sapiens | dual specificity tyrosine phosphorylation regulated kinase 1A(DYRK1A) |
| DYRK1A | Z25423 | Homo sapiens | dual specificity tyrosine phosphorylation regulated kinase 1A(DYRK1A) |
| DYRK1A | BC065184 | Homo sapiens | dual specificity tyrosine phosphorylation regulated kinase 1A(DYRK1A) |
| DYRK1A | NM_001396 | Homo sapiens | dual specificity tyrosine phosphorylation regulated kinase 1A(DYRK1A) |
| DYRK1A | BC156309 | Homo sapiens | dual specificity tyrosine phosphorylation regulated kinase 1A(DYRK1A) |
| DYRK1A | XM_011529485 | Homo sapiens | dual specificity tyrosine phosphorylation regulated kinase 1A(DYRK1A) |
| DYRK1A | AF108830 | Homo sapiens | dual specificity tyrosine phosphorylation regulated kinase 1A(DYRK1A) |
| DYRK1A | XM_011529484 | Homo sapiens | dual specificity tyrosine phosphorylation regulated kinase 1A(DYRK1A) |
| DYRK1A | XM_011529483 | Homo sapiens | dual specificity tyrosine phosphorylation regulated kinase 1A(DYRK1A) |
| DYRK1A | XM_011529482 | Homo sapiens | dual specificity tyrosine phosphorylation regulated kinase 1A(DYRK1A) |
| DYRK1A | XM_006723976 | Homo sapiens | dual specificity tyrosine phosphorylation regulated kinase 1A(DYRK1A) |
| DYRK1A | XM_006723977 | Homo sapiens | dual specificity tyrosine phosphorylation regulated kinase 1A(DYRK1A) |
| DYRK1A | XM_006723978 | Homo sapiens | dual specificity tyrosine phosphorylation regulated kinase 1A(DYRK1A) |
| DYRK1A | XM_006723979 | Homo sapiens | dual specificity tyrosine phosphorylation regulated kinase 1A(DYRK1A) |
| DYRK1A | BC172505 | Homo sapiens | dual specificity tyrosine phosphorylation regulated kinase 1A(DYRK1A) |
| DYRK1A | HF584752 | Homo sapiens | dual specificity tyrosine phosphorylation regulated kinase 1A(DYRK1A) |
| DYRK1A | HF584751 | Homo sapiens | dual specificity tyrosine phosphorylation regulated kinase 1A(DYRK1A) |
| DYRK1A | AK301752 | Homo sapiens | dual specificity tyrosine phosphorylation regulated kinase 1A(DYRK1A) |
| DYRK1A | XM_005260933 | Homo sapiens | dual specificity tyrosine phosphorylation regulated kinase 1A(DYRK1A) |
| DYRK1A | BC030515 | Homo sapiens | dual specificity tyrosine phosphorylation regulated kinase 1A(DYRK1A) |
| DYRK1A | AJ001870 | Homo sapiens | dual specificity tyrosine phosphorylation regulated kinase 1A(DYRK1A) |
| DYRK1A | D86550 | Homo sapiens | dual specificity tyrosine phosphorylation regulated kinase 1A(DYRK1A) |
| DYRK1A | AJ001871 | Homo sapiens | dual specificity tyrosine phosphorylation regulated kinase 1A(DYRK1A) |
| MITF | AB006909 | Homo sapiens | melanogenesis associated transcription factor(MITF) |
| MITF | BQ219650 | Homo sapiens | melanogenesis associated transcription factor(MITF) |
| MITF | AL110195 | Homo sapiens | melanogenesis associated transcription factor(MITF) |
| MITF | BC012503 | Homo sapiens | melanogenesis associated transcription factor(MITF) |
| MITF | AB006988 | Homo sapiens | melanogenesis associated transcription factor(MITF) |
| MITF | NM_001184967 | Homo sapiens | melanogenesis associated transcription factor(MITF) |
| MITF | AB006989 | Homo sapiens | melanogenesis associated transcription factor(MITF) |
| MITF | NM_001184968 | Homo sapiens | melanogenesis associated transcription factor(MITF) |
| MITF | BC026961 | Homo sapiens | melanogenesis associated transcription factor(MITF) |
| MITF | AY632572 | Homo sapiens | melanogenesis associated transcription factor(MITF) |
| MITF | AK291318 | Homo sapiens | melanogenesis associated transcription factor(MITF) |
| MITF | AY632574 | Homo sapiens | melanogenesis associated transcription factor(MITF) |
| MITF | AK297858 | Homo sapiens | melanogenesis associated transcription factor(MITF) |
| MITF | NM_198159 | Homo sapiens | melanogenesis associated transcription factor(MITF) |
| MITF | NM_198158 | Homo sapiens | melanogenesis associated transcription factor(MITF) |
| MITF | BU167035 | Homo sapiens | melanogenesis associated transcription factor(MITF) |
| MITF | AK296129 | Homo sapiens | melanogenesis associated transcription factor(MITF) |
| MITF | NM_198178 | Homo sapiens | melanogenesis associated transcription factor(MITF) |
| MITF | NM_198177 | Homo sapiens | melanogenesis associated transcription factor(MITF) |
| MITF | GU355676 | Homo sapiens | melanogenesis associated transcription factor(MITF) |
| MITF | BC011461 | Homo sapiens | melanogenesis associated transcription factor(MITF) |
| MITF | NM_000248 | Homo sapiens | melanogenesis associated transcription factor(MITF) |
| MITF | XM_005264755 | Homo sapiens | melanogenesis associated transcription factor(MITF) |
| MITF | AB061771 | Homo sapiens | melanogenesis associated transcription factor(MITF) |
| MITF | AL117653 | Homo sapiens | melanogenesis associated transcription factor(MITF) |
| MITF | XM_005264754 | Homo sapiens | melanogenesis associated transcription factor(MITF) |
| MITF | XM_017006448 | Homo sapiens | melanogenesis associated transcription factor(MITF) |
| MITF | BC065243 | Homo sapiens | melanogenesis associated transcription factor(MITF) |
| MITF | XM_017006447 | Homo sapiens | melanogenesis associated transcription factor(MITF) |
| MITF | AW242257 | Homo sapiens | melanogenesis associated transcription factor(MITF) |
| MITF | NM_006722 | Homo sapiens | melanogenesis associated transcription factor(MITF) |
| MITF | XM_017006446 | Homo sapiens | melanogenesis associated transcription factor(MITF) |
| MITF | XM_017006445 | Homo sapiens | melanogenesis associated transcription factor(MITF) |
| MITF | XM_017006444 | Homo sapiens | melanogenesis associated transcription factor(MITF) |
| MITF | DC388606 | Homo sapiens | melanogenesis associated transcription factor(MITF) |
| MITF | DA058963 | Homo sapiens | melanogenesis associated transcription factor(MITF) |
| MITF | BM800230 | Homo sapiens | melanogenesis associated transcription factor(MITF) |
| MITF | XM_011533725 | Homo sapiens | melanogenesis associated transcription factor(MITF) |
| MITF | Z29678 | Homo sapiens | melanogenesis associated transcription factor(MITF) |
| MITF | XM_011533722 | Homo sapiens | melanogenesis associated transcription factor(MITF) |
| MITF | XM_011533723 | Homo sapiens | melanogenesis associated transcription factor(MITF) |
| MITF | XM_006713164 | Homo sapiens | melanogenesis associated transcription factor(MITF) |
| MITF | XM_011533726 | Homo sapiens | melanogenesis associated transcription factor(MITF) |
| PAX8-AS1 | AK056355 | Homo sapiens | PAX8 antisense RNA 1(PAX8-AS1) |
| PAX8-AS1 | BF056746 | Homo sapiens | PAX8 antisense RNA 1(PAX8-AS1) |
| PAX8-AS1 | AK310158 | Homo sapiens | PAX8 antisense RNA 1(PAX8-AS1) |
| PAX8-AS1 | NR_015377 | Homo sapiens | PAX8 antisense RNA 1(PAX8-AS1) |
| PAX8-AS1 | AK307781 | Homo sapiens | PAX8 antisense RNA 1(PAX8-AS1) |
| PAX8-AS1 | AY007128 | Homo sapiens | PAX8 antisense RNA 1(PAX8-AS1) |
| PAX8-AS1 | BC042373 | Homo sapiens | PAX8 antisense RNA 1(PAX8-AS1) |
| PAX8-AS1 | AK056052 | Homo sapiens | PAX8 antisense RNA 1(PAX8-AS1) |
| PAX8-AS1 | LK937783 | Homo sapiens | PAX8 antisense RNA 1(PAX8-AS1) |
| PAX8-AS1 | AK130275 | Homo sapiens | PAX8 antisense RNA 1(PAX8-AS1) |
| PAX8-AS1 | AK126431 | Homo sapiens | PAX8 antisense RNA 1(PAX8-AS1) |
| PAX8-AS1 | AK001856 | Homo sapiens | PAX8 antisense RNA 1(PAX8-AS1) |
| PAX8-AS1 | AL390179 | Homo sapiens | PAX8 antisense RNA 1(PAX8-AS1) |
| PAX8-AS1 | BX537688 | Homo sapiens | PAX8 antisense RNA 1(PAX8-AS1) |
| PAX8-AS1 | CA416382 | Homo sapiens | PAX8 antisense RNA 1(PAX8-AS1) |
| PAX8-AS1 | BC036699 | Homo sapiens | PAX8 antisense RNA 1(PAX8-AS1) |
| PAX8-AS1 | BC033562 | Homo sapiens | PAX8 antisense RNA 1(PAX8-AS1) |
| PAX8-AS1 | DA573927 | Homo sapiens | PAX8 antisense RNA 1(PAX8-AS1) |
| PAX8-AS1 | NR_047570 | Homo sapiens | PAX8 antisense RNA 1(PAX8-AS1) |
| NEAT1 | LK938844 | Homo sapiens | nuclear paraspeckle assembly transcript 1 (non-protein coding)(NEAT1) |
| NEAT1 | LK938846 | Homo sapiens | nuclear paraspeckle assembly transcript 1 (non-protein coding)(NEAT1) |
| NEAT1 | HG503866 | Homo sapiens | nuclear paraspeckle assembly transcript 1 (non-protein coding)(NEAT1) |
| NEAT1 | LK938845 | Homo sapiens | nuclear paraspeckle assembly transcript 1 (non-protein coding)(NEAT1) |
| NEAT1 | EF177379 | Homo sapiens | nuclear paraspeckle assembly transcript 1 (non-protein coding)(NEAT1) |
| NEAT1 | AI590745 | Homo sapiens | nuclear paraspeckle assembly transcript 1 (non-protein coding)(NEAT1) |
| NEAT1 | NR_002802 | Homo sapiens | nuclear paraspeckle assembly transcript 1 (non-protein coding)(NEAT1) |
| NEAT1 | AF508303 | Homo sapiens | nuclear paraspeckle assembly transcript 1 (non-protein coding)(NEAT1) |
| NEAT1 | NR_131012 | Homo sapiens | nuclear paraspeckle assembly transcript 1 (non-protein coding)(NEAT1) |
| NEAT1 | AF080092 | Homo sapiens | nuclear paraspeckle assembly transcript 1 (non-protein coding)(NEAT1) |
| NEAT1 | GQ859162 | Homo sapiens | nuclear paraspeckle assembly transcript 1 (non-protein coding)(NEAT1) |
| NEAT1 | AK027191 | Homo sapiens | nuclear paraspeckle assembly transcript 1 (non-protein coding)(NEAT1) |
| NEAT1 | HG503867 | Homo sapiens | nuclear paraspeckle assembly transcript 1 (non-protein coding)(NEAT1) |
| NEAT1 | U60873 | Homo sapiens | nuclear paraspeckle assembly transcript 1 (non-protein coding)(NEAT1) |
| NEAT1 | NR_028272 | Homo sapiens | nuclear paraspeckle assembly transcript 1 (non-protein coding)(NEAT1) |
| SOX10 | CR536571 | Homo sapiens | SRY-box 10(SOX10) |
| SOX10 | BT020029 | Homo sapiens | SRY-box 10(SOX10) |
| SOX10 | CR456584 | Homo sapiens | SRY-box 10(SOX10) |
| SOX10 | NM_006941 | Homo sapiens | SRY-box 10(SOX10) |
| SOX10 | BC007595 | Homo sapiens | SRY-box 10(SOX10) |
| SOX10 | BC018808 | Homo sapiens | SRY-box 10(SOX10) |
| SOX10 | AK300945 | Homo sapiens | SRY-box 10(SOX10) |
| SOX10 | DQ896471 | Homo sapiens | SRY-box 10(SOX10) |
| SOX10 | DQ893172 | Homo sapiens | SRY-box 10(SOX10) |
| SOX10 | AJ001183 | Homo sapiens | SRY-box 10(SOX10) |
| SOX10 | BC002824 | Homo sapiens | SRY-box 10(SOX10) |
| SOX10 | AK310896 | Homo sapiens | SRY-box 10(SOX10) |
| SOX10 | CU013471 | Homo sapiens | SRY-box 10(SOX10) |
| SOX10 | CU013183 | Homo sapiens | SRY-box 10(SOX10) |
| RUNX2 | XR_926323 | Homo sapiens | runt related transcription factor 2(RUNX2) |
| RUNX2 | NM_001015051 | Homo sapiens | runt related transcription factor 2(RUNX2) |
| RUNX2 | NM_001024630 | Homo sapiens | runt related transcription factor 2(RUNX2) |
| RUNX2 | BC160022 | Homo sapiens | runt related transcription factor 2(RUNX2) |
| RUNX2 | BX108677 | Homo sapiens | runt related transcription factor 2(RUNX2) |
| RUNX2 | XM_006715232 | Homo sapiens | runt related transcription factor 2(RUNX2) |
| RUNX2 | AF053952 | Homo sapiens | runt related transcription factor 2(RUNX2) |
| RUNX2 | BC108919 | Homo sapiens | runt related transcription factor 2(RUNX2) |
| RUNX2 | CN431726 | Homo sapiens | runt related transcription factor 2(RUNX2) |
| RUNX2 | AL353944 | Homo sapiens | runt related transcription factor 2(RUNX2) |
| RUNX2 | L40992 | Homo sapiens | runt related transcription factor 2(RUNX2) |
| RUNX2 | NM_004348 | Homo sapiens | runt related transcription factor 2(RUNX2) |
| RUNX2 | AW469546 | Homo sapiens | runt related transcription factor 2(RUNX2) |
| RUNX2 | NM_001278478 | Homo sapiens | runt related transcription factor 2(RUNX2) |
| RUNX2 | XR_001743701 | Homo sapiens | runt related transcription factor 2(RUNX2) |
| RUNX2 | XM_011514966 | Homo sapiens | runt related transcription factor 2(RUNX2) |
| RUNX2 | AF087960 | Homo sapiens | runt related transcription factor 2(RUNX2) |
| RUNX2 | XM_011514965 | Homo sapiens | runt related transcription factor 2(RUNX2) |
| RUNX2 | XM_011514964 | Homo sapiens | runt related transcription factor 2(RUNX2) |
| RUNX2 | XM_011514963 | Homo sapiens | runt related transcription factor 2(RUNX2) |
| RUNX2 | BC108920 | Homo sapiens | runt related transcription factor 2(RUNX2) |
| RUNX2 | XM_011514962 | Homo sapiens | runt related transcription factor 2(RUNX2) |
| RUNX2 | XM_011514961 | Homo sapiens | runt related transcription factor 2(RUNX2) |
| RUNX2 | XM_017011391 | Homo sapiens | runt related transcription factor 2(RUNX2) |
| RUNX2 | XM_011514960 | Homo sapiens | runt related transcription factor 2(RUNX2) |
| RUNX2 | NR_103533 | Homo sapiens | runt related transcription factor 2(RUNX2) |
| RUNX2 | XM_017011394 | Homo sapiens | runt related transcription factor 2(RUNX2) |
| RUNX2 | XM_017011395 | Homo sapiens | runt related transcription factor 2(RUNX2) |
| RUNX2 | XM_017011392 | Homo sapiens | runt related transcription factor 2(RUNX2) |
| RUNX2 | XM_017011393 | Homo sapiens | runt related transcription factor 2(RUNX2) |
| RUNX2 | CD001961 | Homo sapiens | runt related transcription factor 2(RUNX2) |
| RUNX2 | DR005078 | Homo sapiens | runt related transcription factor 2(RUNX2) |
| RUNX2 | XM_017011396 | Homo sapiens | runt related transcription factor 2(RUNX2) |
| RUNX2 | NR_103532 | Homo sapiens | runt related transcription factor 2(RUNX2) |
| MYL4 | XM_005257391 | Homo sapiens | myosin light chain 4(MYL4) |
| MYLA | XM_011524839 | Homo sapiens | myosin light chain 4(MYL4) |
| MYL4 | NM_002476 | Homo sapiens | myosin light chain 4(MYL4) |
| MYL4 | XM_017024683 | Homo sapiens | myosin light chain 4(MYL4) |
| MYL4 | X13955 | Homo sapiens | myosin light chain 4(MYL4) |
| MYL4 | X52005 | Homo sapiens | myosin light chain 4(MYL4) |
| MYL4 | AF116676 | Homo sapiens | myosin light chain 4(MYL4) |
| MYL4 | XM_017024684 | Homo sapiens | myosin light chain 4(MYL4) |
| MYL4 | BC030228 | Homo sapiens | myosin light chain 4(MYL4) |
| MYL4 | M36172 | Homo sapiens | myosin light chain 4(MYL4) |
| MYL4 | H83803 | Homo sapiens | myosin light chain 4(MYL4) |
| MYL4 | M20641 | Homo sapiens | myosin light chain 4(MYL4) |
| MYL4 | AM392902 | Homo sapiens | myosin light chain 4(MYL4) |
| MYL4 | AM392536 | Homo sapiens | myosin light chain 4(MYL4) |
| MYL4 | AM392523 | Homo sapiens | myosin light chain 4(MYL4) |
| MYLA | AM393643 | Homo sapiens | myosin light chain 4(MYL4) |
| MYL4 | NM_001002841 | Homo sapiens | myosin light chain 4(MYL4) |
| MYL4 | AM393677 | Homo sapiens | myosin light chain 4(MYL4) |
| MYL4 | BU658678 | Homo sapiens | myosin light chain 4(MYL4) |
| MYL4 | M24121 | Homo sapiens | myosin light chain 4(MYL4) |
| LMX1A | NM_177399 | Homo sapiens | LIM homeobox transcription factor 1 alpha(LMX1A) |
| LMX1A | NM_177398 | Homo sapiens | LIM homeobox transcription factor 1 alpha(LMX1A) |
| LMX1A | AK122800 | Homo sapiens | LIM homeobox transcription factor 1 alpha(LMX1A) |
| LMX1A | NM_001033507 | Homo sapiens | LIM homeobox transcription factor 1 alpha(LMX1A) |
| LMX1A | BC119744 | Homo sapiens | LIM homeobox transcription factor 1 alpha(LMX1A) |
| LMX1A | BC119743 | Homo sapiens | LIM homeobox transcription factor 1 alpha(LMX1A) |
| LMX1A | XM_011509538 | Homo sapiens | LIM homeobox transcription factor 1 alpha(LMX1A) |
| LMX1A | BC066353 | Homo sapiens | LIM homeobox transcription factor 1 alpha(LMX1A) |
| LMX1A | NM_001174069 | Homo sapiens | LIM homeobox transcription factor 1 alpha(LMX1A) |
| LMX1A | XM_011509540 | Homo sapiens | LIM homeobox transcription factor 1 alpha(LMX1A) |
| LMX1A | BM678780 | Homo sapiens | LIM homeobox transcription factor 1 alpha(LMX1A) |
| LMX1A | AK127724 | Homo sapiens | LIM homeobox transcription factor 1 alpha(LMX1A) |
| LMX1A | JF432394 | Homo sapiens | LIM homeobox transcription factor 1 alpha(LMX1A) |
| LMX1A | BC160062 | Homo sapiens | LIM homeobox transcription factor 1 alpha(LMX1A) |
| PSME4 | XM_011532709 | Homo sapiens | proteasome activator subunit 4(PSME4) |
| PSME4 | AK026085 | Homo sapiens | proteasome activator subunit 4(PSME4) |
| PSME4 | XM_011532707 | Homo sapiens | proteasome activator subunit 4(PSME4) |
| PSME4 | AA884260 | Homo sapiens | proteasome activator subunit 4(PSME4) |
| PSME4 | BC043602 | Homo sapiens | proteasome activator subunit 4(PSME4) |
| PSME4 | AK124923 | Homo sapiens | proteasome activator subunit 4(PSME4) |
| PSME4 | AU117034 | Homo sapiens | proteasome activator subunit 4(PSME4) |
| PSME4 | AB621805 | Homo sapiens | proteasome activator subunit 4(PSME4) |
| PSME4 | AL045471 | Homo sapiens | proteasome activator subunit 4(PSME4) |
| PSME4 | BC143737 | Homo sapiens | proteasome activator subunit 4(PSME4) |
| PSME4 | BC062760 | Homo sapiens | proteasome activator subunit 4(PSME4) |
| PSME4 | BC143739 | Homo sapiens | proteasome activator subunit 4(PSME4) |
| PSME4 | NM_014614 | Homo sapiens | proteasome activator subunit 4(PSME4) |
| PSME4 | AK025517 | Homo sapiens | proteasome activator subunit 4(PSME4) |
| PSME4 | BC112169 | Homo sapiens | proteasome activator subunit 4(PSME4) |
| PSME4 | AL599601 | Homo sapiens | proteasome activator subunit 4(PSME4) |
| PSME4 | AY894754 | Homo sapiens | proteasome activator subunit 4(PSME4) |
| PSME4 | D38521 | Homo sapiens | proteasome activator subunit 4(PSME4) |
| PSME4 | XM_011532705 | Homo sapiens | proteasome activator subunit 4(PSME4) |
| PSME4 | XM_011532706 | Homo sapiens | proteasome activator subunit 4(PSME4) |
| PSME4 | XR_001738679 | Homo sapiens | proteasome activator subunit 4(PSME4) |
| PSME4 | AY894755 | Homo sapiens | proteasome activator subunit 4(PSME4) |
| PSME4 | AY894756 | Homo sapiens | proteasome activator subunit 4(PSME4) |
| PSME4 | BC071768 | Homo sapiens | proteasome activator subunit 4(PSME4) |
| PSME4 | BQ898809 | Homo sapiens | proteasome activator subunit 4(PSME4) |
| PSME4 | XM_006711969 | Homo sapiens | proteasome activator subunit 4(PSME4) |
| PSME4 | BU569553 | Homo sapiens | proteasome activator subunit 4(PSME4) |
| PSME4 | BC032418 | Homo sapiens | proteasome activator subunit 4(PSME4) |
| PSME4 | BC017090 | Homo sapiens | proteasome activator subunit 4(PSME4) |
| PSME4 | BC113668 | Homo sapiens | proteasome activator subunit 4(PSME4) |
| CFTR | BC156254 | Homo sapiens | cystic fibrosis transmembrane conductance regulator(CFTR) |
| CFTR | XM_017011699 | Homo sapiens | cystic fibrosis transmembrane conductance regulator(CFTR) |
| CFTR | X73053 | Homo sapiens | cystic fibrosis transmembrane conductance regulator(CFTR) |
| CFTR | XM_011515754 | Homo sapiens | cystic fibrosis transmembrane conductance regulator(CFTR) |
| CFTR | M28668 | Homo sapiens | cystic fibrosis transmembrane conductance regulator(CFTR) |
| CFTR | XM_011515753 | Homo sapiens | cystic fibrosis transmembrane conductance regulator(CFTR) |
| CFTR | S64699 | Homo sapiens | cystic fibrosis transmembrane conductance regulator(CFTR) |
| CFTR | XM_011515751 | Homo sapiens | cystic fibrosis transmembrane conductance regulator(CFTR) |
| CFTR | NM_000492 | Homo sapiens | cystic fibrosis transmembrane conductance regulator(CFTR) |
| CFTR | S82430 | Homo sapiens | cystic fibrosis transmembrane conductance regulator(CFTR) |
| CFTR | BC143713 | Homo sapiens | cystic fibrosis transmembrane conductance regulator(CFTR) |
| CNTN4-AS2 | BE041831 | Homo sapiens | cystic fibrosis transmembrane conductance regulator(CFTR) |
| CNTN4-AS2 | NR_046555 | Homo sapiens | CNTN4 antisense RNA 2(CNTN4-AS2) |
| CNTN4-AS2 | HG495393 | Homo sapiens | CNTN4 antisense RNA 2(CNTN4-AS2) |
| CHRNA1 | Y00762 | Homo sapiens | cholinergic receptor nicotinic alpha 1 subunit(CHRNA1) |
| CHRNA1 | CD013889 | Homo sapiens | cholinergic receptor nicotinic alpha 1 subunit(CHRNA1) |
| CHRNA1 | AK299445 | Homo sapiens | cholinergic receptor nicotinic alpha 1 subunit(CHRNA1) |
| CHRNA1 | NM_000079 | Homo sapiens | cholinergic receptor nicotinic alpha 1 subunit(CHRNA1) |
| CHRNA1 | XM_017003257 | Homo sapiens | cholinergic receptor nicotinic alpha 1 subunit(CHRNA1) |
| CHRNA1 | XM_017003256 | Homo sapiens | cholinergic receptor nicotinic alpha 1 subunit(CHRNA1) |
| CHRNA1 | BG828551 | Homo sapiens | cholinergic receptor nicotinic alpha 1 subunit(CHRNA1) |
| CHRNA1 | NM_001039523 | Homo sapiens | cholinergic receptor nicotinic alpha 1 subunit(CHRNA1) |
| CHRNA1 | BC043196 | Homo sapiens | cholinergic receptor nicotinic alpha 1 subunit(CHRNA1) |
| CHRNA1 | AK291338 | Homo sapiens | cholinergic receptor nicotinic alpha 1 subunit(CHRNA1) |
| CHRNA1 | BC006314 | Homo sapiens | cholinergic receptor nicotinic alpha 1 subunit(CHRNA1) |
| CHRNA1 | AK315312 | Homo sapiens | cholinergic receptor nicotinic alpha 1 subunit(CHRNA1) |
| CHRNA1 | DQ323657 | Homo sapiens | cholinergic receptor nicotinic alpha 1 subunit(CHRNA1) |
| CHRNA1 | DQ323658 | Homo sapiens | cholinergic receptor nicotinic alpha 1 subunit(CHRNA1) |
| CHRNA1 | S77094 | Homo sapiens | cholinergic receptor nicotinic alpha 1 subunit(CHRNA1) |
| NOTCH1 | AB209873 | Homo sapiens | notch 1(NOTCH1) |
| NOTCH1 | AF308602 | Homo sapiens | notch 1(NOTCH1) |
| NOTCH1 | CR457221 | Homo sapiens | notch 1(NOTCH1) |
| NOTCH1 | R42303 | Homo sapiens | notch 1(NOTCH1) |
| NOTCH1 | AK000012 | Homo sapiens | notch 1(NOTCH1) |
| NOTCH1 | BC046127 | Homo sapiens | notch 1(NOTCH1) |
| NOTCH1 | BC013208 | Homo sapiens | notch 1(NOTCH1) |
| NOTCH1 | BC049843 | Homo sapiens | notch 1(NOTCH1) |
| NOTCH1 | M73980 | Homo sapiens | notch 1(NOTCH1) |
| NOTCH1 | BC063597 | Homo sapiens | notch 1(NOTCH1) |
| NOTCH1 | CN431067 | Homo sapiens | notch 1(NOTCH1) |
| NOTCH1 | BC039147 | Homo sapiens | notch 1(NOTCH1) |
| NOTCH1 | DA324222 | Homo sapiens | notch 1(NOTCH1) |
| NOTCH1 | NM_017617 | Homo sapiens | notch 1(NOTCH1) |
| NOTCH1 | XM_011518717 | Homo sapiens | notch 1(NOTCH1) |
| PDE1A | XM_017004295 | Homo sapiens | phosphodiesterase 1A(PDE1A) |
| PDE1A | XM_017004296 | Homo sapiens | phosphodiesterase 1A(PDE1A) |
| PDE1A | DA128734 | Homo sapiens | phosphodiesterase 1A(PDE1A) |
| PDE1A | XM_017004294 | Homo sapiens | phosphodiesterase 1A(PDE1A) |
| PDE1A | AF110240 | Homo sapiens | phosphodiesterase 1A(PDE1A) |
| PDE1A | BG196993 | Homo sapiens | phosphodiesterase 1A(PDE1A) |
| PDE1A | AK301720 | Homo sapiens | phosphodiesterase 1A(PDE1A) |
| PDE1A | DB128091 | Homo sapiens | phosphodiesterase 1A(PDE1A) |
| PDE1A | XM_017004299 | Homo sapiens | phosphodiesterase 1A(PDE1A) |
| PDE1A | XM_017004297 | Homo sapiens | phosphodiesterase 1A(PDE1A) |
| PDE1A | XM_017004298 | Homo sapiens | phosphodiesterase 1A(PDE1A) |
| PDE1A | XM_011511323 | Homo sapiens | phosphodiesterase 1A(PDE1A) |
| PDE1A | BM719913 | Homo sapiens | phosphodiesterase 1A(PDE1A) |
| PDE1A | XM_011511325 | Homo sapiens | phosphodiesterase 1A(PDE1A) |
| PDE1A | XM_011511324 | Homo sapiens | phosphodiesterase 1A(PDE1A) |
| PDEIA | AJ401610 | Homo sapiens | phosphodiesterase 1A(PDE1A) |
| PDE1A | AK295657 | Homo sapiens | phosphodiesterase 1A(PDE1A) |
| PDE1A | DQ896694 | Homo sapiens | phosphodiesterase 1A(PDE1A) |
| PDE1A | XM_011511326 | Homo sapiens | phosphodiesterase 1A(PDE1A) |
| PDE1A | AA846454 | Homo sapiens | phosphodiesterase 1A(PDE1A) |
| PDE1A | DQ892369 | Homo sapiens | phosphodiesterase 1A(PDE1A) |
| PDE1A | NM_001003683 | Homo sapiens | phosphodiesterase 1A(PDE1A) |
| PDE1A | U40370 | Homo sapiens | phosphodiesterase 1A(PDE1A) |
| PDE1A | AL110263 | Homo sapiens | phosphodiesterase 1A(PDE1A) |
| PDE1A | NM_005019 | Homo sapiens | phosphodiesterase 1A(PDE1A) |
| PDE1A | XM_017004301 | Homo sapiens | phosphodiesterase 1A(PDE1A) |
| PDE1A | AF110238 | Homo sapiens | phosphodiesterase 1A(PDE1A) |
| PDE1A | XM_017004302 | Homo sapiens | phosphodiesterase 1A(PDE1A) |
| PDE1A | AF110237 | Homo sapiens | phosphodiesterase 1A(PDE1A) |
| PDE1A | AF110236 | Homo sapiens | phosphodiesterase 1A(PDE1A) |
| PDE1A | XM_017004300 | Homo sapiens | phosphodiesterase 1A(PDE1A) |
| PDE1A | AF110235 | Homo sapiens | phosphodiesterase 1A(PDE1A) |
| PDE1A | AL536937 | Homo sapiens | phosphodiesterase 1A(PDE1A) |
| PDE1A | BC047057 | Homo sapiens | phosphodiesterase 1A(PDE1A) |
| PDE1A | NM_001258313 | Homo sapiens | phosphodiesterase 1A(PDE1A) |
| PDE1A | DB512285 | Homo sapiens | phosphodiesterase 1A(PDE1A) |
| PDE1A | NM_001258312 | Homo sapiens | phosphodiesterase 1A(PDE1A) |
| PDE1A | BC022480 | Homo sapiens | phosphodiesterase 1A(PDE1A) |
| PDE1A | DA295167 | Homo sapiens | phosphodiesterase 1A(PDE1A) |
| PDE1A | DC396228 | Homo sapiens | phosphodiesterase 1A(PDE1A) |
| PDE1A | AK130643 | Homo sapiens | phosphodiesterase 1A(PDE1A) |
| PDE1A | NM_001258314 | Homo sapiens | phosphodiesterase 1A(PDE1A) |
| PDE1A | AK294239 | Homo sapiens | phosphodiesterase 1A(PDE1A) |
| PDE1A | AB038227 | Homo sapiens | phosphodiesterase 1A(PDE1A) |
| PDE1A | DQ893372 | Homo sapiens | phosphodiesterase 1A(PDE1A) |
| PDE1A | DQ895578 | Homo sapiens | phosphodiesterase 1A(PDE1A) |
| PDE1A | AB038228 | Homo sapiens | phosphodiesterase 1A(PDE1A) |
| PDE1A | XR_001738769 | Homo sapiens | phosphodiesterase 1A(PDE1A) |
| ARX | BC169333 | Homo sapiens | aristaless related homeobox(ARX) |
| ARX | BC169334 | Homo sapiens | aristaless related homeobox(ARX) |
| ARX | AY038071 | Homo sapiens | aristaless related homeobox(ARX) |
| ARX | AA484051 | Homo sapiens | aristaless related homeobox(ARX) |
| ARX | BQ269551 | Homo sapiens | aristaless related homeobox(ARX) |
| ARX | NM_139058 | Homo sapiens | aristaless related homeobox(ARX) |
| ARX | BQ100952 | Homo sapiens | aristaless related homeobox(ARX) |
| ARX | BF196892 | Homo sapiens | aristaless related homeobox(ARX) |
| ARX | CA775911 | Homo sapiens | aristaless related homeobox(ARX) |
| GLIS3 | DQ438889 | Homo sapiens | GLIS family zinc finger 3(GLIS3) |
| GLIS3 | DQ438888 | Homo sapiens | GLIS family zinc finger 3(GLIS3) |
| GLIS3 | DQ438885 | Homo sapiens | GLIS family zinc finger 3(GLIS3) |
| GLIS3 | DQ438884 | Homo sapiens | GLIS family zinc finger 3(GLIS3) |
| GLIS3 | DQ438887 | Homo sapiens | GLIS family zinc finger 3(GLIS3) |
| GLIS3 | DQ438886 | Homo sapiens | GLIS family zinc finger 3(GLIS3) |
| GLIS3 | DQ438881 | Homo sapiens | GLIS family zinc finger 3(GLIS3) |
| GLIS3 | DQ438880 | Homo sapiens | GLIS family zinc finger 3(GLIS3) |
| GLIS3 | DQ438883 | Homo sapiens | GLIS family zinc finger 3(GLIS3) |
| GLIS3 | DQ438882 | Homo sapiens | GLIS family zinc finger 3(GLIS3) |
| GLIS3 | NM_152629 | Homo sapiens | GLIS family zinc finger 3(GLIS3) |
| GLIS3 | AK096318 | Homo sapiens | GLIS family zinc finger 3(GLIS3) |
| GLIS3 | AK075059 | Homo sapiens | GLIS family zinc finger 3(GLIS3) |
| GLIS3 | XM_011517764 | Homo sapiens | GLIS family zinc finger 3(GLIS3) |
| GLIS3 | XM_011517765 | Homo sapiens | GLIS family zinc finger 3(GLIS3) |
| GLIS3 | XM_011517766 | Homo sapiens | GLIS family zinc finger 3(GLIS3) |
| GLIS3 | XM_011517767 | Homo sapiens | GLIS family zinc finger 3(GLIS3) |
| GLIS3 | XM_011517769 | Homo sapiens | GLIS family zinc finger 3(GLIS3) |
| GLIS3 | DQ438899 | Homo sapiens | GLIS family zinc finger 3(GLIS3) |
| GLIS3 | DQ438896 | Homo sapiens | GLIS family zinc finger 3(GLIS3) |
| GLIS3 | DQ438895 | Homo sapiens | GLIS family zinc finger 3(GLIS3) |
| GLIS3 | KU178885 | Homo sapiens | GLIS family zinc finger 3(GLIS3) |
| GLIS3 | DQ438898 | Homo sapiens | GLIS family zinc finger 3(GLIS3) |
| GLIS3 | KU178886 | Homo sapiens | GLIS family zinc finger 3(GLIS3) |
| GLIS3 | DQ438897 | Homo sapiens | GLIS family zinc finger 3(GLIS3) |
| GLIS3 | KU178887 | Homo sapiens | GLIS family zinc finger 3(GLIS3) |
| GLIS3 | DQ438892 | Homo sapiens | GLIS family zinc finger 3(GLIS3) |
| GLIS3 | XM_005251387 | Homo sapiens | GLIS family zinc finger 3(GLIS3) |
| GLIS3 | DQ438891 | Homo sapiens | GLIS family zinc finger 3(GLIS3) |
| GLIS3 | XM_005251386 | Homo sapiens | GLIS family zinc finger 3(GLIS3) |
| GLIS3 | DQ438894 | Homo sapiens | GLIS family zinc finger 3(GLIS3) |
| GLIS3 | DQ438893 | Homo sapiens | GLIS family zinc finger 3(GLIS3) |
| GLIS3 | DQ438890 | Homo sapiens | GLIS family zinc finger 3(GLIS3) |
| GLIS3 | XM_017014361 | Homo sapiens | GLIS family zinc finger 3(GLIS3) |
| GLIS3 | XM_005251389 | Homo sapiens | GLIS family zinc finger 3(GLIS3) |
| GLIS3 | XM_005251388 | Homo sapiens | GLIS family zinc finger 3(GLIS3) |
| GLIS3 | EU446681 | Homo sapiens | GLIS family zinc finger 3(GLIS3) |
| GLIS3 | DQ438900 | Homo sapiens | GLIS family zinc finger 3(GLIS3) |
| GLIS3 | AB065086 | Homo sapiens | GLIS family zinc finger 3(GLIS3) |
| GLIS3 | DQ438902 | Homo sapiens | GLIS family zinc finger 3(GLIS3) |
| GLIS3 | DQ438901 | Homo sapiens | GLIS family zinc finger 3(GLIS3) |
| GLIS3 | AA933816 | Homo sapiens | GLIS family zinc finger 3(GLIS3) |
| GLIS3 | AK055907 | Homo sapiens | GLIS family zinc finger 3(GLIS3) |
| GLIS3 | BC033899 | Homo sapiens | GLIS family zinc finger 3(GLIS3) |
| GLIS3 | DQ438907 | Homo sapiens | GLIS family zinc finger 3(GLIS3) |
| GLIS3 | HG501866 | Homo sapiens | GLIS family zinc finger 3(GLIS3) |
| GLIS3 | DQ438904 | Homo sapiens | GLIS family zinc finger 3(GLIS3) |
| GLIS3 | DQ438903 | Homo sapiens | GLIS family zinc finger 3(GLIS3) |
| GLIS3 | DQ438906 | Homo sapiens | GLIS family zinc finger 3(GLIS3) |
| GLIS3 | DQ438905 | Homo sapiens | GLIS family zinc finger 3(GLIS3) |
| GLIS3 | DQ438878 | Homo sapiens | GLIS family zinc finger 3(GLIS3) |
| GLIS3 | DQ438877 | Homo sapiens | GLIS family zinc finger 3(GLIS3) |
| GLIS3 | DQ438879 | Homo sapiens | GLIS family zinc finger 3(GLIS3) |
| GLIS3 | AB209404 | Homo sapiens | GLIS family zinc finger 3(GLIS3) |
| GLIS3 | XM_011517763 | Homo sapiens | GLIS family zinc finger 3(GLIS3) |
| GLIS3 | XR_929206 | Homo sapiens | GLIS family zinc finger 3(GLIS3) |
| GLIS3 | XM_006716731 | Homo sapiens | GLIS family zinc finger 3(GLIS3) |
| GLIS3 | NM_001042413 | Homo sapiens | GLIS family zinc finger 3(GLIS3) |
| TWIST1 | Y11177 | Homo sapiens | twist family bHLH transcription factor 1(TWIST1) |
| TWIST1 | Y11178 | Homo sapiens | twist family bHLH transcription factor 1(TWIST1) |
| TWIST1 | AW173505 | Homo sapiens | twist family bHLH transcription factor 1(TWIST1) |
| TWIST1 | X99268 | Homo sapiens | twist family bHLH transcription factor 1(TWIST1) |
| TWIST1 | BC036704 | Homo sapiens | twist family bHLH transcription factor 1(TWIST1) |
| TWIST1 | NM_000474 | Homo sapiens | twist family bHLH transcription factor 1(TWIST1) |
| TWIST1 | XM_011515496 | Homo sapiens | twist family bHLH transcription factor 1(TWIST1) |
| TWIST1 | DQ896770 | Homo sapiens | twist family bHLH transcription factor 1(TWIST1) |
| PHYHIPL | DB551000 | Homo sapiens | phytanoyl-CoA 2-hydroxylase interacting protein like(PHYHIPL) |
| PHYHIPL | BC011268 | Homo sapiens | phytanoyl-CoA 2-hydroxylase interacting protein like(PHYHIPL) |
| PHYHIPL | AM393181 | Homo sapiens | phytanoyl-CoA 2-hydroxylase interacting protein like(PHYHIPL) |
| PHYHIPL | DB455338 | Homo sapiens | phytanoyl-CoA 2-hydroxylase interacting protein like(PHYHIPL) |
| PHYHIPL | AY358162 | Homo sapiens | phytanoyl-CoA 2-hydroxylase interacting protein like(PHYHIPL) |
| PHYHIPL | AW299583 | Homo sapiens | phytanoyl-CoA 2-hydroxylase interacting protein like(PHYHIPL) |
| PHYHIPL | NM_001143774 | Homo sapiens | phytanoyl-CoA 2-hydroxylase interacting protein like(PHYHIPL) |
| PHYHIPL | AB058699 | Homo sapiens | phytanoyl-CoA 2-hydroxylase interacting protein like(PHYHIPL) |
| PHYHIPL | CR749429 | Homo sapiens | phytanoyl-CoA 2-hydroxylase interacting protein like(PHYHIPL) |
| PHYHIPL | AL365474 | Homo sapiens | phytanoyl-CoA 2-hydroxylase interacting protein like(PHYHIPL) |
| PHYHIPL | AL834339 | Homo sapiens | phytanoyl-CoA 2-hydroxylase interacting protein like(PHYHIPL) |
| PHYHIPL | AM392743 | Homo sapiens | phytanoyl-CoA 2-hydroxylase interacting protein like(PHYHIPL) |
| PHYHIPL | BM676577 | Homo sapiens | phytanoyl-CoA 2-hydroxylase interacting protein like(PHYHIPL) |
| PHYHIPL | NM_032439 | Homo sapiens | phytanoyl-CoA 2-hydroxylase interacting protein like(PHYHIPL) |
| PHYHIPL | XM_011540276 | Homo sapiens | phytanoyl-CoA 2-hydroxylase interacting protein like(PHYHIPL) |
| PHYHIPL | XM_011540275 | Homo sapiens | phytanoyl-CoA 2-hydroxylase interacting protein like(PHYHIPL) |
| PHYHIPL | XM_017016783 | Homo sapiens | phytanoyl-CoA 2-hydroxylase interacting protein like(PHYHIPL) |
| PHYHIPL | XM_017016782 | Homo sapiens | phytanoyl-CoA 2-hydroxylase interacting protein like(PHYHIPL) |
| PHYHIPL | AK054956 | Homo sapiens | phytanoyl-CoA 2-hydroxylase interacting protein like(PHYHIPL) |
| TRPM1 | BU933033 | Homo sapiens | transient receptor potential cation channel subfamily M member 1(TRPM1) |
| TRPM1 | BC058286 | Homo sapiens | transient receptor potential cation channel subfamily M member 1(TRPM1) |
| TRPM1 | GQ502181 | Homo sapiens | transient receptor potential cation channel subfamily M member 1(TRPM1) |
| TRPM1 | BC005892 | Homo sapiens | transient receptor potential cation channel subfamily M member 1(TRPM1) |
| TRPM1 | AB115498 | Homo sapiens | transient receptor potential cation channel subfamily M member 1(TRPM1) |
| TRPM1 | AB115500 | Homo sapiens | transient receptor potential cation channel subfamily M member 1(TRPM1) |
| TRPM1 | NM_001252030 | Homo sapiens | transient receptor potential cation channel subfamily M member 1(TRPM1) |
| TRPM1 | AB115501 | Homo sapiens | transient receptor potential cation channel subfamily M member 1(TRPM1) |
| TRPM1 | BC156069 | Homo sapiens | transient receptor potential cation channel subfamily M member 1(TRPM1) |
| TRPM1 | AB115502 | Homo sapiens | transient receptor potential cation channel subfamily M member 1(TRPM1) |
| TRPM1 | GU576175 | Homo sapiens | transient receptor potential cation channel subfamily M member 1(TRPM1) |
| TRPM1 | N42519 | Homo sapiens | transient receptor potential cation channel subfamily M member 1(TRPM1) |
| TRPM1 | BM695497 | Homo sapiens | transient receptor potential cation channel subfamily M member 1(TRPM1) |
| TRPM1 | NM_002420 | Homo sapiens | transient receptor potential cation channel subfamily M member 1(TRPM1) |
| TRPM1 | NM_001252024 | Homo sapiens | transient receptor potential cation channel subfamily M member 1(TRPM1) |
| TRPM1 | BC017849 | Homo sapiens | transient receptor potential cation channel subfamily M member 1(TRPM1) |
| TRPM1 | NM_001252020 | Homo sapiens | transient receptor potential cation channel subfamily M member 1(TRPM1) |
| TRPM1 | AB115499 | Homo sapiens | transient receptor potential cation channel subfamily M member 1(TRPM1) |
| TRPM1 | BC033627 | Homo sapiens | transient receptor potential cation channel subfamily M member 1(TRPM1) |
| TRPM1 | AF071787 | Homo sapiens | transient receptor potential cation channel subfamily M member 1(TRPM1) |
| TRPM1 | HM135791 | Homo sapiens | transient receptor potential cation channel subfamily M member 1(TRPM1) |
| TRPM1 | BC070356 | Homo sapiens | transient receptor potential cation channel subfamily M member 1(TRPM1) |
| TRPM1 | HM135790 | Homo sapiens | transient receptor potential cation channel subfamily M member 1(TRPM1) |
| TRPM1 | BU743036 | Homo sapiens | transient receptor potential cation channel subfamily M member 1(TRPM1) |
| TRPM1 | BU741333 | Homo sapiens | transient receptor potential cation channel subfamily M member 1(TRPM1) |
| ADGRG2 | BC063315 | Homo sapiens | adhesion G protein-coupled receptor G2(ADGRG2) |
| ADGRG2 | AF539455 | Homo sapiens | adhesion G protein-coupled receptor G2(ADGRG2) |
| ADGRG2 | AF539456 | Homo sapiens | adhesion G protein-coupled receptor G2(ADGRG2) |
| ADGRG2 | NM_001079860 | Homo sapiens | adhesion G protein-coupled receptor G2(ADGRG2) |
| ADGRG2 | XM_006724455 | Homo sapiens | adhesion G protein-coupled receptor G2(ADGRG2) |
| ADGRG2 | NM_001184837 | Homo sapiens | adhesion G protein-coupled receptor G2(ADGRG2) |
| ADGRG2 | AF538954 | Homo sapiens | adhesion G protein-coupled receptor G2(ADGRG2) |
| ADGRG2 | AK291012 | Homo sapiens | adhesion G protein-coupled receptor G2(ADGRG2) |
| ADGRG2 | AY148343 | Homo sapiens | adhesion G protein-coupled receptor G2(ADGRG2) |
| ADGRG2 | BC099901 | Homo sapiens | adhesion G protein-coupled receptor G2(ADGRG2) |
| ADGRG2 | BC113979 | Homo sapiens | adhesion G protein-coupled receptor G2(ADGRG2) |
| ADGRG2 | BC113978 | Homo sapiens | adhesion G protein-coupled receptor G2(ADGRG2) |
| ADGRG2 | XM_011545434 | Homo sapiens | adhesion G protein-coupled receptor G2(ADGRG2) |
| ADGRG2 | XM_011545435 | Homo sapiens | adhesion G protein-coupled receptor G2(ADGRG2) |
| ADGRG2 | NM_001184833 | Homo sapiens | adhesion G protein-coupled receptor G2(ADGRG2) |
| ADGRG2 | NM_001184834 | Homo sapiens | adhesion G protein-coupled receptor G2(ADGRG2) |
| ADGRG2 | NM_005756 | Homo sapiens | adhesion G protein-coupled receptor G2(ADGRG2) |
| ADGRG2 | NM_001184835 | Homo sapiens | adhesion G protein-coupled receptor G2(ADGRG2) |
| ADGRG2 | NM_001184836 | Homo sapiens | adhesion G protein-coupled receptor G2(ADGRG2) |
| ADGRG2 | AK309439 | Homo sapiens | adhesion G protein-coupled receptor G2(ADGRG2) |
| ADGRG2 | NM_001079858 | Homo sapiens | adhesion G protein-coupled receptor G2(ADGRG2) |
| ADGRG2 | NM_001079859 | Homo sapiens | adhesion G protein-coupled receptor G2(ADGRG2) |
| ADGRG2 | AY143364 | Homo sapiens | adhesion G protein-coupled receptor G2(ADGRG2) |
| ADGRG2 | AY143366 | Homo sapiens | adhesion G protein-coupled receptor G2(ADGRG2) |
| ADGRG2 | AY143365 | Homo sapiens | adhesion G protein-coupled receptor G2(ADGRG2) |
| ADGRG2 | AY143367 | Homo sapiens | adhesion G protein-coupled receptor G2(ADGRG2) |
| ADGRG2 | X81892 | Homo sapiens | adhesion G protein-coupled receptor G2(ADGRG2) |
| ADGRG2 | AK090402 | Homo sapiens | adhesion G protein-coupled receptor G2(ADGRG2) |
| GALR1 | AY541036 | Homo sapiens | galanin receptor 1(GALR1) |
| GALR1 | NM_001480 | Homo sapiens | galanin receptor 1(GALR1) |
| GALR1 | BC095530 | Homo sapiens | galanin receptor 1(GALR1) |
| GALR1 | U23854 | Homo sapiens | galanin receptor 1(GALR1) |
| GALR1 | XM_017025691 | Homo sapiens | galanin receptor 1(GALR1) |
| GALR1 | L34339 | Homo sapiens | galanin receptor 1(GALR1) |
| GALR1 | U53511 | Homo sapiens | galanin receptor 1(GALR1) |
| MBP | XM_011526009 | Homo sapiens | myelin basic protein(MBP) |
| MBP | BC030093 | Homo sapiens | myelin basic protein(MBP) |
| MBP | NM_001025100 | Homo sapiens | myelin basic protein(MBP) |
| MBP | CR627018 | Homo sapiens | myelin basic protein(MBP) |
| MBP | BC101771 | Homo sapiens | myelin basic protein(MBP) |
| MBP | BC101773 | Homo sapiens | myelin basic protein(MBP) |
| MBP | CR541919 | Homo sapiens | myelin basic protein(MBP) |
| MBP | AK094611 | Homo sapiens | myelin basic protein(MBP) |
| MBP | AK128788 | Homo sapiens | myelin basic protein(MBP) |
| MBP | BC068550 | Homo sapiens | myelin basic protein(MBP) |
| MBP | AK314553 | Homo sapiens | myelin basic protein(MBP) |
| MBP | CB156561 | Homo sapiens | myelin basic protein(MBP) |
| MBP | BU430656 | Homo sapiens | myelin basic protein(MBP) |
| MBP | NM_001025101 | Homo sapiens | myelin basic protein(MBP) |
| MBP | BC065248 | Homo sapiens | myelin basic protein(MBP) |
| MBP | M30515 | Homo sapiens | myelin basic protein(MBP) |
| MBP | AK126858 | Homo sapiens | myelin basic protein(MBP) |
| MBP | CR536534 | Homo sapiens | myelin basic protein(MBP) |
| MBP | BG766942 | Homo sapiens | myelin basic protein(MBP) |
| MBP | N20370 | Homo sapiens | myelin basic protein(MBP) |
| MBP | M20009 | Homo sapiens | myelin basic protein(MBP) |
| MBP | AK074315 | Homo sapiens | myelin basic protein(MBP) |
| MBP | BC008749 | Homo sapiens | myelin basic protein(MBP) |
| MBP | AK124830 | Homo sapiens | myelin basic protein(MBP) |
| MBP | AK293922 | Homo sapiens | myelin basic protein(MBP) |
| MBP | M30516 | Homo sapiens | myelin basic protein(MBP) |
| MBP | M30047 | Homo sapiens | myelin basic protein(MBP) |
| MBP | AB208986 | Homo sapiens | myelin basic protein(MBP) |
| MBP | AK289893 | Homo sapiens | myelin basic protein(MBP) |
| MBP | AK095121 | Homo sapiens | myelin basic protein(MBP) |
| MBP | NM_001025081 | Homo sapiens | myelin basic protein(MBP) |
| MBP | BC080654 | Homo sapiens | myelin basic protein(MBP) |
| MBP | XM_017025779 | Homo sapiens | myelin basic protein(MBP) |
| MBP | XM_017025778 | Homo sapiens | myelin basic protein(MBP) |
| MBP | BC130034 | Homo sapiens | myelin basic protein(MBP) |
| MBP | AK093588 | Homo sapiens | myelin basic protein(MBP) |
| MBP | NM_002385 | Homo sapiens | myelin basic protein(MBP) |
| MBP | AK098513 | Homo sapiens | myelin basic protein(MBP) |
| MBP | AK123433 | Homo sapiens | myelin basic protein(MBP) |
| MBP | L18865 | Homo sapiens | myelin basic protein(MBP) |
| MBP | NM_001025094 | Homo sapiens | myelin basic protein(MBP) |
| MBP | XR_001753201 | Homo sapiens | myelin basic protein(MBP) |
| MBP | XR_001753202 | Homo sapiens | myelin basic protein(MBP) |
| MBP | AK296492 | Homo sapiens | myelin basic protein(MBP) |
| MBP | NM_001025098 | Homo sapiens | myelin basic protein(MBP) |
| MBP | BC143348 | Homo sapiens | myelin basic protein(MBP) |
| MBP | M13577 | Homo sapiens | myelin basic protein(MBP) |
| MBP | XM_017025780 | Homo sapiens | myelin basic protein(MBP) |
| MBP | NM_001025092 | Homo sapiens | myelin basic protein(MBP) |
| MBP | CB153535 | Homo sapiens | myelin basic protein(MBP) |
| MBP | NM_001025090 | Homo sapiens | myelin basic protein(MBP) |
| MBP | AK098402 | Homo sapiens | myelin basic protein(MBP) |
| MBP | BM977768 | Homo sapiens | myelin basic protein(MBP) |
| MBP | AK128770 | Homo sapiens | myelin basic protein(MBP) |
| MBP | AK122594 | Homo sapiens | myelin basic protein(MBP) |
| MBP | BC143350 | Homo sapiens | myelin basic protein(MBP) |
| OTX2 | NM_001270524 | Homo sapiens | orthodenticle homeobox 2(OTX2) |
| OTX2 | BU176852 | Homo sapiens | orthodenticle homeobox 2(OTX2) |
| OTX2 | DQ890875 | Homo sapiens | orthodenticle homeobox 2(OTX2) |
| OTX2 | NM_001270523 | Homo sapiens | orthodenticle homeobox 2(OTX2) |
| OTX2 | BE781530 | Homo sapiens | orthodenticle homeobox 2(OTX2) |
| OTX2 | DB294481 | Homo sapiens | orthodenticle homeobox 2(OTX2) |
| OTX2 | NM_001270525 | Homo sapiens | orthodenticle homeobox 2(OTX2) |
| OTX2 | NM_021728 | Homo sapiens | orthodenticle homeobox 2(OTX2) |
| OTX2 | NM_172337 | Homo sapiens | orthodenticle homeobox 2(OTX2) |
| OTX2 | EU176492 | Homo sapiens | orthodenticle homeobox 2(OTX2) |
| OTX2 | CV812961 | Homo sapiens | orthodenticle homeobox 2(OTX2) |
| OTX2 | AB593056 | Homo sapiens | orthodenticle homeobox 2(OTX2) |
| OTX2 | AK314271 | Homo sapiens | orthodenticle homeobox 2(OTX2) |
| OTX2 | NR_073034 | Homo sapiens | orthodenticle homeobox 2(OTX2) |
| OTX2 | NR_073036 | Homo sapiens | orthodenticle homeobox 2(OTX2) |
| OTX2 | BC032579 | Homo sapiens | orthodenticle homeobox 2(OTX2) |
| OTX2 | AF093138 | Homo sapiens | orthodenticle homeobox 2(OTX2) |
| OTX2 | AB593058 | Homo sapiens | orthodenticle homeobox 2(OTX2) |
| OTX2 | AB593057 | Homo sapiens | orthodenticle homeobox 2(OTX2) |
| PIP5K1B | CB988216 | Homo sapiens | phosphatidylinositol-4-phosphate 5-kinase type 1 beta(PIP5K1B) |
| PIP5K1B | NM_001031687 | Homo sapiens | phosphatidylinositol-4-phosphate 5-kinase type 1 beta(PIP5K1B) |
| PIP5K1B | DQ890874 | Homo sapiens | phosphatidylinositol-4-phosphate 5-kinase type 1 beta(PIP5K1B) |
| PIP5K1B | XM_006717300 | Homo sapiens | phosphatidylinositol-4-phosphate 5-kinase type 1 beta(PIP5K1B) |
| PIP5K1B | XM_006717301 | Homo sapiens | phosphatidylinositol-4-phosphate 5-kinase type 1 beta(PIP5K1B) |
| PIP5K1B | XM_011519082 | Homo sapiens | phosphatidylinositol-4-phosphate 5-kinase type 1 beta(PIP5K1B) |
| PIP5K1B | NM_003558 | Homo sapiens | phosphatidylinositol-4-phosphate 5-kinase type 1 beta(PIP5K1B) |
| PIP5K1B | U78579 | Homo sapiens | phosphatidylinositol-4-phosphate 5-kinase type 1 beta(PIP5K1B) |
| PIP5K1B | XM_011519084 | Homo sapiens | phosphatidylinositol-4-phosphate 5-kinase type 1 beta(PIP5K1B) |
| PIP5K1B | XM_005252262 | Homo sapiens | phosphatidylinositol-4-phosphate 5-kinase type 1 beta(PIP5K1B) |
| PIP5K1B | XM_011519083 | Homo sapiens | phosphatidylinositol-4-phosphate 5-kinase type 1 beta(PIP5K1B) |
| PIP5K1B | XM_005252261 | Homo sapiens | phosphatidylinositol-4-phosphate 5-kinase type 1 beta(PIP5K1B) |
| PIP5K1B | NM_001278253 | Homo sapiens | phosphatidylinositol-4-phosphate 5-kinase type 1 beta(PIP5K1B) |
| PIP5K1B | BC030587 | Homo sapiens | phosphatidylinositol-4-phosphate 5-kinase type 1 beta(PIP5K1B) |
| PIP5K1B | DQ894030 | Homo sapiens | phosphatidylinositol-4-phosphate 5-kinase type 1 beta(PIP5K1B) |
| PIP5K1B | XM_017015190 | Homo sapiens | phosphatidylinositol-4-phosphate 5-kinase type 1 beta(PIP5K1B) |
| PIP5K1B | U78581 | Homo sapiens | phosphatidylinositol-4-phosphate 5-kinase type 1 beta(PIP5K1B) |
| PIP5K1B | XM_017015191 | Homo sapiens | phosphatidylinositol-4-phosphate 5-kinase type 1 beta(PIP5K1B) |
| PIP5K1B | U78580 | Homo sapiens | phosphatidylinositol-4-phosphate 5-kinase type 1 beta(PIP5K1B) |
| PIP5K1B | AK292734 | Homo sapiens | phosphatidylinositol-4-phosphate 5-kinase type 1 beta(PIP5K1B) |
| PIP5K1B | XM_017015188 | Homo sapiens | phosphatidylinositol-4-phosphate 5-kinase type 1 beta(PIP5K1B) |
| PIP5K1B | AK295587 | Homo sapiens | phosphatidylinositol-4-phosphate 5-kinase type 1 beta(PIP5K1B) |
| PIP5K1B | CK299479 | Homo sapiens | phosphatidylinositol-4-phosphate 5-kinase type 1 beta(PIP5K1B) |
| PIP5K1B | XM_017015189 | Homo sapiens | phosphatidylinositol-4-phosphate 5-kinase type 1 beta(PIP5K1B) |
| OTX1 | NR_130153 | Homo sapiens | orthodenticle homeobox 1(OTX1) |
| OTX1 | DQ896509 | Homo sapiens | orthodenticle homeobox 1(OTX1) |
| OTX1 | BU689444 | Homo sapiens | orthodenticle homeobox 1(OTX1) |
| OTX1 | NM_001199770 | Homo sapiens | orthodenticle homeobox 1(OTX1) |
| OTX1 | AK095680 | Homo sapiens | orthodenticle homeobox 1(OTX1) |
| OTX1 | NM_014562 | Homo sapiens | orthodenticle homeobox 1(OTX1) |
| OTX1 | AK297308 | Homo sapiens | orthodenticle homeobox 1(OTX1) |
| OTX1 | BC007621 | Homo sapiens | orthodenticle homeobox 1(OTX1) |
| OTX1 | BP310240 | Homo sapiens | orthodenticle homeobox 1(OTX1) |
| OTX1 | BM720707 | Homo sapiens | orthodenticle homeobox 1(OTX1) |
| OTX1 | DQ893301 | Homo sapiens | orthodenticle homeobox 1(OTX1) |
| FAM83D | BC001068 | Homo sapiens | family with sequence similarity 83 member D(FAM83D) |
| FAM83D | XM_017028088 | Homo sapiens | family with sequence similarity 83 member D(FAM83D) |
| FAM83D | AK303860 | Homo sapiens | family with sequence similarity 83 member D(FAM83D) |
| FAM83D | NM_030919 | Homo sapiens | family with sequence similarity 83 member D(FAM83D) |
| FAM83D | AK055793 | Homo sapiens | family with sequence similarity 83 member D(FAM83D) |
| FAM83D | AL832274 | Homo sapiens | family with sequence similarity 83 member D(FAM83D) |
| FAM83D | BC080188 | Homo sapiens | family with sequence similarity 83 member D(FAM83D) |
| FAM83D | AK095660 | Homo sapiens | family with sequence similarity 83 member D(FAM83D) |
| FAM83D | BC006553 | Homo sapiens | family with sequence similarity 83 member D(FAM83D) |
| FAM83D | BC063661 | Homo sapiens | family with sequence similarity 83 member D(FAM83D) |
| FAM83D | BC053683 | Homo sapiens | family with sequence similarity 83 member D(FAM83D) |
| TRPM3 | XM_011519035 | Homo sapiens | transient receptor potential cation channel subfamily M member 3(TRPM3) |
| TRPM3 | XM_011519037 | Homo sapiens | transient receptor potential cation channel subfamily M member 3(TRPM3) |
| TRPM3 | XM_011519036 | Homo sapiens | transient receptor potential cation channel subfamily M member 3(TRPM3) |
| TRPM3 | AL136545 | Homo sapiens | transient receptor potential cation channel subfamily M member 3(TRPM3) |
| TRPM3 | XM_011519039 | Homo sapiens | transient receptor potential cation channel subfamily M member 3(TRPM3) |
| TRPM3 | XM_011519038 | Homo sapiens | transient receptor potential cation channel subfamily M member 3(TRPM3) |
| TRPM3 | BC121821 | Homo sapiens | transient receptor potential cation channel subfamily M member 3(TRPM3) |
| TRPM3 | BC067733 | Homo sapiens | transient receptor potential cation channel subfamily M member 3(TRPM3) |
| TRPM3 | XM_017015141 | Homo sapiens | transient receptor potential cation channel subfamily M member 3(TRPM3) |
| TRPM3 | XM_017015142 | Homo sapiens | transient receptor potential cation channel subfamily M member 3(TRPM3) |
| TRPM3 | BC134414 | Homo sapiens | transient receptor potential cation channel subfamily M member 3(TRPM3) |
| TRPM3 | NM_001007470 | Homo sapiens | transient receptor potential cation channel subfamily M member 3(TRPM3) |
| TRPM3 | NM_001007471 | Homo sapiens | transient receptor potential cation channel subfamily M member 3(TRPM3) |
| TRPM3 | XM_017015147 | Homo sapiens | transient receptor potential cation channel subfamily M member 3(TRPM3) |
| TRPM3 | XM_017015148 | Homo sapiens | transient receptor potential cation channel subfamily M member 3(TRPM3) |
| TRPM3 | AK308682 | Homo sapiens | transient receptor potential cation channel subfamily M member 3(TRPM3) |
| TRPM3 | XM_017015149 | Homo sapiens | transient receptor potential cation channel subfamily M member 3(TRPM3) |
| TRPM3 | XM_017015143 | Homo sapiens | transient receptor potential cation channel subfamily M member 3(TRPM3) |
| TRPM3 | XM_017015144 | Homo sapiens | transient receptor potential cation channel subfamily M member 3(TRPM3) |
| TRPM3 | XM_017015145 | Homo sapiens | transient receptor potential cation channel subfamily M member 3(TRPM3) |
| TRPM3 | XM_017015146 | Homo sapiens | transient receptor potential cation channel subfamily M member 3(TRPM3) |
| TRPM3 | AK225732 | Homo sapiens | transient receptor potential cation channel subfamily M member 3(TRPM3) |
| TRPM3 | XM_011519044 | Homo sapiens | transient receptor potential cation channel subfamily M member 3(TRPM3) |
| TRPM3 | XM_011519043 | Homo sapiens | transient receptor potential cation channel subfamily M member 3(TRPM3) |
| TRPM3 | XM_011519046 | Homo sapiens | transient receptor potential cation channel subfamily M member 3(TRPM3) |
| TRPM3 | XM_011519045 | Homo sapiens | transient receptor potential cation channel subfamily M member 3(TRPM3) |
| TRPM3 | XM_011519047 | Homo sapiens | transient receptor potential cation channel subfamily M member 3(TRPM3) |
| TRPM3 | AB099665 | Homo sapiens | transient receptor potential cation channel subfamily M member 3(TRPM3) |
| TRPM3 | AB099663 | Homo sapiens | transient receptor potential cation channel subfamily M member 3(TRPM3) |
| TRPM3 | AB099664 | Homo sapiens | transient receptor potential cation channel subfamily M member 3(TRPM3) |
| TRPM3 | AB099661 | Homo sapiens | transient receptor potential cation channel subfamily M member 3(TRPM3) |
| TRPM3 | NM_020952 | Homo sapiens | transient receptor potential cation channel subfamily M member 3(TRPM3) |
| TRPM3 | XM_011519040 | Homo sapiens | transient receptor potential cation channel subfamily M member 3(TRPM3) |
| TRPM3 | AB099662 | Homo sapiens | transient receptor potential cation channel subfamily M member 3(TRPM3) |
| TRPM3 | XM_011519042 | Homo sapiens | transient receptor potential cation channel subfamily M member 3(TRPM3) |
| TRPM3 | XM_011519041 | Homo sapiens | transient receptor potential cation channel subfamily M member 3(TRPM3) |
| TRPM3 | AK021788 | Homo sapiens | transient receptor potential cation channel subfamily M member 3(TRPM3) |
| TRPM3 | CD673832 | Homo sapiens | transient receptor potential cation channel subfamily M member 3(TRPM3) |
| TRPM3 | AF325212 | Homo sapiens | transient receptor potential cation channel subfamily M member 3(TRPM3) |
| TRPM3 | AJ505026 | Homo sapiens | transient receptor potential cation channel subfamily M member 3(TRPM3) |
| TRPM3 | AJ505025 | Homo sapiens | transient receptor potential cation channel subfamily M member 3(TRPM3) |
| TRPM3 | BC172350 | Homo sapiens | transient receptor potential cation channel subfamily M member 3(TRPM3) |
| TRPM3 | BM668925 | Homo sapiens | transient receptor potential cation channel subfamily M member 3(TRPM3) |
| TRPM3 | NM_206947 | Homo sapiens | transient receptor potential cation channel subfamily M member 3(TRPM3) |
| TRPM3 | XM_017015161 | Homo sapiens | transient receptor potential cation channel subfamily M member 3(TRPM3) |
| TRPM3 | NM_206948 | Homo sapiens | transient receptor potential cation channel subfamily M member 3(TRPM3) |
| TRPM3 | NM_206944 | Homo sapiens | transient receptor potential cation channel subfamily M member 3(TRPM3) |
| TRPM3 | NM_206945 | Homo sapiens | transient receptor potential cation channel subfamily M member 3(TRPM3) |
| TRPM3 | NM_206946 | Homo sapiens | transient receptor potential cation channel subfamily M member 3(TRPM3) |
| TRPM3 | XM_017015160 | Homo sapiens | transient receptor potential cation channel subfamily M member 3(TRPM3) |
| TRPM3 | AF536752 | Homo sapiens | transient receptor potential cation channel subfamily M member 3(TRPM3) |
| TRPM3 | AF536751 | Homo sapiens | transient receptor potential cation channel subfamily M member 3(TRPM3) |
| TRPM3 | AF536753 | Homo sapiens | transient receptor potential cation channel subfamily M member 3(TRPM3) |
| TRPM3 | BC094699 | Homo sapiens | transient receptor potential cation channel subfamily M member 3(TRPM3) |
| TRPM3 | AF536750 | Homo sapiens | transient receptor potential cation channel subfamily M member 3(TRPM3) |
| TRPM3 | BC142972 | Homo sapiens | transient receptor potential cation channel subfamily M member 3(TRPM3) |
| TRPM3 | NM_024971 | Homo sapiens | transient receptor potential cation channel subfamily M member 3(TRPM3) |
| TRPM3 | BC022454 | Homo sapiens | transient receptor potential cation channel subfamily M member 3(TRPM3) |
| TRPM3 | AB046836 | Homo sapiens | transient receptor potential cation channel subfamily M member 3(TRPM3) |
| TRPM3 | CD104154 | Homo sapiens | transient receptor potential cation channel subfamily M member 3(TRPM3) |
| TRPM3 | KF987075 | Homo sapiens | transient receptor potential cation channel subfamily M member 3(TRPM3) |
| TRPM3 | AF536749 | Homo sapiens | transient receptor potential cation channel subfamily M member 3(TRPM3) |
| TRPM3 | XM_017015150 | Homo sapiens | transient receptor potential cation channel subfamily M member 3(TRPM3) |
| TRPM3 | AF536748 | Homo sapiens | transient receptor potential cation channel subfamily M member 3(TRPM3) |
| TRPM3 | XM_017015151 | Homo sapiens | transient receptor potential cation channel subfamily M member 3(TRPM3) |
| TRPM3 | XM_017015152 | Homo sapiens | transient receptor potential cation channel subfamily M member 3(TRPM3) |
| TRPM3 | XM_017015153 | Homo sapiens | transient receptor potential cation channel subfamily M member 3(TRPM3) |
| TRPM3 | XM_017015158 | Homo sapiens | transient receptor potential cation channel subfamily M member 3(TRPM3) |
| TRPM3 | XM_017015159 | Homo sapiens | transient receptor potential cation channel subfamily M member 3(TRPM3) |
| TRPM3 | XM_017015154 | Homo sapiens | transient receptor potential cation channel subfamily M member 3(TRPM3) |
| TRPM3 | XM_017015155 | Homo sapiens | transient receptor potential cation channel subfamily M member 3(TRPM3) |
| TRPM3 | XM_017015156 | Homo sapiens | transient receptor potential cation channel subfamily M member 3(TRPM3) |
| TRPM3 | XM_017015157 | Homo sapiens | transient receptor potential cation channel subfamily M member 3(TRPM3) |
| HSPA9 | AU130219 | Homo sapiens | heat shock protein family A (Hsp70) member 9(HSPA9) |
| HSPA9 | DQ185038 | Homo sapiens | heat shock protein family A (Hsp70) member 9(HSPA9) |
| HSPA9 | AK225488 | Homo sapiens | heat shock protein family A (Hsp70) member 9(HSPA9) |
| HSPA9 | AK297795 | Homo sapiens | heat shock protein family A (Hsp70) member 9(HSPA9) |
| HSPA9 | AK293990 | Homo sapiens | heat shock protein family A (Hsp70) member 9(HSPA9) |
| HSPA9 | DQ480334 | Homo sapiens | heat shock protein family A (Hsp70) member 9(HSPA9) |
| HSPA9 | BC024034 | Homo sapiens | heat shock protein family A (Hsp70) member 9(HSPA9) |
| HSPA9 | FJ224291 | Homo sapiens | heat shock protein family A (Hsp70) member 9(HSPA9) |
| HSPA9 | AK023317 | Homo sapiens | heat shock protein family A (Hsp70) member 9(HSPA9) |
| HSPA9 | BC000478 | Homo sapiens | heat shock protein family A (Hsp70) member 9(HSPA9) |
| HSPA9 | BX426279 | Homo sapiens | heat shock protein family A (Hsp70) member 9(HSPA9) |
| HSPA9 | AK315177 | Homo sapiens | heat shock protein family A (Hsp70) member 9(HSPA9) |
| HSPA9 | BC030634 | Homo sapiens | heat shock protein family A (Hsp70) member 9(HSPA9) |
| HSPA9 | NM_004134 | Homo sapiens | heat shock protein family A (Hsp70) member 9(HSPA9) |
| HSPA9 | AK222758 | Homo sapiens | heat shock protein family A (Hsp70) member 9(HSPA9) |
| HSPA9 | EU446981 | Homo sapiens | heat shock protein family A (Hsp70) member 9(HSPA9) |
| HSPA9 | JF432370 | Homo sapiens | heat shock protein family A (Hsp70) member 9(HSPA9) |
| HSPA9 | L15189 | Homo sapiens | heat shock protein family A (Hsp70) member 9(HSPA9) |
| HSPA9 | DC356852 | Homo sapiens | heat shock protein family A (Hsp70) member 9(HSPA9) |
| HSPA9 | L11066 | Homo sapiens | heat shock protein family A (Hsp70) member 9(HSPA9) |
| HSPA9 | AK297897 | Homo sapiens | heat shock protein family A (Hsp70) member 9(HSPA9) |
| TFAP2B | AK291172 | Homo sapiens | transcription factor AP-2 beta(TFAP2B) |
| TFAP2B | AK313749 | Homo sapiens | transcription factor AP-2 beta(TFAP2B) |
| TFAP2B | BC037225 | Homo sapiens | transcription factor AP-2 beta(TFAP2B) |
| TFAP2B | DQ891443 | Homo sapiens | transcription factor AP-2 beta(TFAP2B) |
| TFAP2B | XM_017011235 | Homo sapiens | transcription factor AP-2 beta(TFAP2B) |
| TFAP2B | BU738725 | Homo sapiens | transcription factor AP-2 beta(TFAP2B) |
| TFAP2B | NM_003221 | Homo sapiens | transcription factor AP-2 beta(TFAP2B) |
| TFAP2B | X95694 | Homo sapiens | transcription factor AP-2 beta(TFAP2B) |
| TFAP2B | DQ894621 | Homo sapiens | transcription factor AP-2 beta(TFAP2B) |
| TFAP2B | XM_017011233 | Homo sapiens | transcription factor AP-2 beta(TFAP2B) |
| TFAP2B | AU141084 | Homo sapiens | transcription factor AP-2 beta(TFAP2B) |
| TFAP2B | XM_011514837 | Homo sapiens | transcription factor AP-2 beta(TFAP2B) |
| TFAP2B | XM_017011234 | Homo sapiens | transcription factor AP-2 beta(TFAP2B) |
| FZD3 | AK291480 | Homo sapiens | frizzled class receptor 3(FZD3) |
| FZD3 | BU676090 | Homo sapiens | frizzled class receptor 3(FZD3) |
| FZD3 | XM_017013844 | Homo sapiens | frizzled class receptor 3(FZD3) |
| FZD3 | XM_017013843 | Homo sapiens | frizzled class receptor 3(FZD3) |
| FZD3 | XM_017013842 | Homo sapiens | frizzled class receptor 3(FZD3) |
| FZD3 | NM_017412 | Homo sapiens | frizzled class receptor 3(FZD3) |
| FZD3 | XR_001745597 | Homo sapiens | frizzled class receptor 3(FZD3) |
| FZD3 | BC042009 | Homo sapiens | frizzled class receptor 3(FZD3) |
| FZD3 | XR_949476 | Homo sapiens | frizzled class receptor 3(FZD3) |
| FZD3 | BC172481 | Homo sapiens | frizzled class receptor 3(FZD3) |
| FZD3 | BC156293 | Homo sapiens | frizzled class receptor 3(FZD3) |
| FZD3 | AY005130 | Homo sapiens | frizzled class receptor 3(FZD3) |
| FZD3 | NM_145866 | Homo sapiens | frizzled class receptor 3(FZD3) |
| FZD3 | AB039723 | Homo sapiens | frizzled class receptor 3(FZD3) |
| FZD3 | XM_017013841 | Homo sapiens | frizzled class receptor 3(FZD3) |
| FZD3 | BC034278 | Homo sapiens | frizzled class receptor 3(FZD3) |
| FZD3 | AJ272427 | Homo sapiens | frizzled class receptor 3(FZD3) |
| FZD3 | DA250029 | Homo sapiens | frizzled class receptor 3(FZD3) |
| DNAH11 | BQ365981 | Homo sapiens | dynein axonemal heavy chain 11(DNAH11) |
| DNAH11 | JQ247523 | Homo sapiens | dynein axonemal heavy chain 11(DNAH11) |
| DNAH11 | AK095018 | Homo sapiens | dynein axonemal heavy chain 11(DNAH11) |
| DNAH11 | AL529367 | Homo sapiens | dynein axonemal heavy chain 11(DNAH11) |
| DNAH11 | CA424782 | Homo sapiens | dynein axonemal heavy chain 11(DNAH11) |
| DNAH11 | JQ247524 | Homo sapiens | dynein axonemal heavy chain 11(DNAH11) |
| DNAH11 | AJ132087 | Homo sapiens | dynein axonemal heavy chain 11(DNAH11) |
| DNAH11 | AK054657 | Homo sapiens | dynein axonemal heavy chain 11(DNAH11) |
| DNAH11 | NM_001277115 | Homo sapiens | dynein axonemal heavy chain 11(DNAH11) |
| DNAH11 | AJ320497 | Homo sapiens | dynein axonemal heavy chain 11(DNAH11) |
| DNAH11 | AI743846 | Homo sapiens | dynein axonemal heavy chain 11(DNAH11) |
| DNAH11 | BF369731 | Homo sapiens | dynein axonemal heavy chain 11(DNAH11) |
| PLK1 | AB209179 | Homo sapiens | polo like kinase 1(PLK1) |
| PLK1 | AK303263 | Homo sapiens | polo like kinase 1(PLK1) |
| PLK1 | AK308276 | Homo sapiens | polo like kinase 1(PLK1) |
| PLK1 | X75932 | Homo sapiens | polo like kinase 1(PLK1) |
| PLK1 | BC014846 | Homo sapiens | polo like kinase 1(PLK1) |
| PLK1 | X73458 | Homo sapiens | polo like kinase 1(PLK1) |
| PLK1 | DC300189 | Homo sapiens | polo like kinase 1(PLK1) |
| PLK1 | AB084459 | Homo sapiens | polo like kinase 1(PLK1) |
| PLK1 | NM_005030 | Homo sapiens | polo like kinase 1(PLK1) |
| PLK1 | BC003002 | Homo sapiens | polo like kinase 1(PLK1) |
| PLK1 | BC002369 | Homo sapiens | polo like kinase 1(PLK1) |
| PLK1 | L19559 | Homo sapiens | polo like kinase 1(PLK1) |
| PLK1 | U01038 | Homo sapiens | polo like kinase 1(PLK1) |
| PLK1 | DQ891045 | Homo sapiens | polo like kinase 1(PLK1) |
| PLK1 | AK313227 | Homo sapiens | polo like kinase 1(PLK1) |
| PLK1 | DQ894224 | Homo sapiens | polo like kinase 1(PLK1) |
| FN1 | AF130095 | Homo sapiens | fibronectin 1(FN1) |
| FN1 | AJ849445 | Homo sapiens | fibronectin 1(FN1) |
| FN1 | BX641150 | Homo sapiens | fibronectin 1(FN1) |
| FN1 | CA422234 | Homo sapiens | fibronectin 1(FN1) |
| FN1 | NM_054034 | Homo sapiens | fibronectin 1(FN1) |
| FN1 | AF312399 | Homo sapiens | fibronectin 1(FN1) |
| FN1 | BX538018 | Homo sapiens | fibronectin 1(FN1) |
| FN1 | U41724 | Homo sapiens | fibronectin 1(FN1) |
| FN1 | BX538017 | Homo sapiens | fibronectin 1(FN1) |
| FN1 | NM_001306129 | Homo sapiens | fibronectin 1(FN1) |
| FN1 | BC016875 | Homo sapiens | fibronectin 1(FN1) |
| FN1 | CR749281 | Homo sapiens | fibronectin 1(FN1) |
| FN1 | EU831936 | Homo sapiens | fibronectin 1(FN1) |
| FN1 | NM_001306132 | Homo sapiens | fibronectin 1(FN1) |
| FN1 | NM_001306130 | Homo sapiens | fibronectin 1(FN1) |
| FN1 | XM_005246408 | Homo sapiens | fibronectin 1(FN1) |
| FN1 | NM_001306131 | Homo sapiens | fibronectin 1(FN1) |
| FN1 | XM_005246407 | Homo sapiens | fibronectin 1(FN1) |
| FN1 | NM_002026 | Homo sapiens | fibronectin 1(FN1) |
| FN1 | XM_005246406 | Homo sapiens | fibronectin 1(FN1) |
| FN1 | XM_005246405 | Homo sapiens | fibronectin 1(FN1) |
| FN1 | XM_005246409 | Homo sapiens | fibronectin 1(FN1) |
| FN1 | BX640875 | Homo sapiens | fibronectin 1(FN1) |
| FN1 | CR749317 | Homo sapiens | fibronectin 1(FN1) |
| FN1 | BX640638 | Homo sapiens | fibronectin 1(FN1) |
| FN1 | CR749316 | Homo sapiens | fibronectin 1(FN1) |
| FN1 | XM_005246404 | Homo sapiens | fibronectin 1(FN1) |
| FN1 | XM_005246403 | Homo sapiens | fibronectin 1(FN1) |
| FN1 | BX640999 | Homo sapiens | fibronectin 1(FN1) |
| FN1 | XM_005246402 | Homo sapiens | fibronectin 1(FN1) |
| FN1 | XM_005246401 | Homo sapiens | fibronectin 1(FN1) |
| FN1 | M27590 | Homo sapiens | fibronectin 1(FN1) |
| FN1 | XM_005246416 | Homo sapiens | fibronectin 1(FN1) |
| FN1 | AK300216 | Homo sapiens | fibronectin 1(FN1) |
| FN1 | AK300458 | Homo sapiens | fibronectin 1(FN1) |
| FN1 | XM_005246411 | Homo sapiens | fibronectin 1(FN1) |
| FN1 | XM_005246410 | Homo sapiens | fibronectin 1(FN1) |
| FN1 | AK316350 | Homo sapiens | fibronectin 1(FN1) |
| FN1 | AL832202 | Homo sapiens | fibronectin 1(FN1) |
| FN1 | XM_005246414 | Homo sapiens | fibronectin 1(FN1) |
| FN1 | CR749666 | Homo sapiens | fibronectin 1(FN1) |
| FN1 | XM_005246412 | Homo sapiens | fibronectin 1(FN1) |
| FN1 | M27589 | Homo sapiens | fibronectin 1(FN1) |
| FN1 | U42455 | Homo sapiens | fibronectin 1(FN1) |
| FN1 | BC117176 | Homo sapiens | fibronectin 1(FN1) |
| FN1 | BX537590 | Homo sapiens | fibronectin 1(FN1) |
| FN1 | U42456 | Homo sapiens | fibronectin 1(FN1) |
| FN1 | U42457 | Homo sapiens | fibronectin 1(FN1) |
| FN1 | U42458 | Homo sapiens | fibronectin 1(FN1) |
| FN1 | BX538045 | Homo sapiens | fibronectin 1(FN1) |
| FN1 | BX640608 | Homo sapiens | fibronectin 1(FN1) |
| FN1 | BX640731 | Homo sapiens | fibronectin 1(FN1) |
| FN1 | AB191261 | Homo sapiens | fibronectin 1(FN1) |
| FN1 | M10905 | Homo sapiens | fibronectin 1(FN1) |
| FN1 | BQ005645 | Homo sapiens | fibronectin 1(FN1) |
| FN1 | BC078656 | Homo sapiens | fibronectin 1(FN1) |
| FN1 | X02761 | Homo sapiens | fibronectin 1(FN1) |
| FN1 | EF550130 | Homo sapiens | fibronectin 1(FN1) |
| FN1 | BT006856 | Homo sapiens | fibronectin 1(FN1) |
| FN1 | AI033037 | Homo sapiens | fibronectin 1(FN1) |
| FN1 | CR936623 | Homo sapiens | fibronectin 1(FN1) |
| FN1 | XM_005246399 | Homo sapiens | fibronectin 1(FN1) |
| FN1 | XM_005246398 | Homo sapiens | fibronectin 1(FN1) |
| FN1 | XM_005246397 | Homo sapiens | fibronectin 1(FN1) |
| FN1 | U42592 | Homo sapiens | fibronectin 1(FN1) |
| FN1 | U42593 | Homo sapiens | fibronectin 1(FN1) |
| FN1 | U42594 | Homo sapiens | fibronectin 1(FN1) |
| FN1 | AK297733 | Homo sapiens | fibronectin 1(FN1) |
| FN1 | BC143763 | Homo sapiens | fibronectin 1(FN1) |
| FN1 | EF550135 | Homo sapiens | fibronectin 1(FN1) |
| FN1 | EF550134 | Homo sapiens | fibronectin 1(FN1) |
| FN1 | EF550133 | Homo sapiens | fibronectin 1(FN1) |
| FN1 | EF550132 | Homo sapiens | fibronectin 1(FN1) |
| FN1 | EF550131 | Homo sapiens | fibronectin 1(FN1) |
| FN1 | AL832771 | Homo sapiens | fibronectin 1(FN1) |
| FN1 | NM_212476 | Homo sapiens | fibronectin 1(FN1) |
| FN1 | NM_212478 | Homo sapiens | fibronectin 1(FN1) |
| FN1 | AJ320527 | Homo sapiens | fibronectin 1(FN1) |
| FN1 | U42404 | Homo sapiens | fibronectin 1(FN1) |
| FN1 | AJ320526 | Homo sapiens | fibronectin 1(FN1) |
| FN1 | BC143754 | Homo sapiens | fibronectin 1(FN1) |
| FN1 | AJ320525 | Homo sapiens | fibronectin 1(FN1) |
| FN1 | DA851446 | Homo sapiens | fibronectin 1(FN1) |
| FN1 | AK094153 | Homo sapiens | fibronectin 1(FN1) |
| FN1 | BX640920 | Homo sapiens | fibronectin 1(FN1) |
| FN1 | BX640803 | Homo sapiens | fibronectin 1(FN1) |
| FN1 | BX640802 | Homo sapiens | fibronectin 1(FN1) |
| FN1 | U60068 | Homo sapiens | fibronectin 1(FN1) |
| FN1 | U60067 | Homo sapiens | fibronectin 1(FN1) |
| FN1 | BC100030 | Homo sapiens | fibronectin 1(FN1) |
| FN1 | NM_212482 | Homo sapiens | fibronectin 1(FN1) |
| FN1 | EU832031 | Homo sapiens | fibronectin 1(FN1) |
| FN1 | AB209840 | Homo sapiens | fibronectin 1(FN1) |
| FN1 | BX649182 | Homo sapiens | fibronectin 1(FN1) |
| FN1 | AK300246 | Homo sapiens | fibronectin 1(FN1) |
| FN1 | AJ276395 | Homo sapiens | fibronectin 1(FN1) |
| FN1 | XM_017003693 | Homo sapiens | fibronectin 1(FN1) |
| FN1 | XM_017003692 | Homo sapiens | fibronectin 1(FN1) |
| FN1 | AB209287 | Homo sapiens | fibronectin 1(FN1) |
| FN1 | XM_017003695 | Homo sapiens | fibronectin 1(FN1) |
| FN1 | XM_017003694 | Homo sapiens | fibronectin 1(FN1) |
| FN1 | AJ535086 | Homo sapiens | fibronectin 1(FN1) |
| FN1 | BC005858 | Homo sapiens | fibronectin 1(FN1) |
| FN1 | AK026737 | Homo sapiens | fibronectin 1(FN1) |
| FN1 | NM_212474 | Homo sapiens | fibronectin 1(FN1) |
| FN1 | U41850 | Homo sapiens | fibronectin 1(FN1) |
| FN1 | NM_212475 | Homo sapiens | fibronectin 1(FN1) |
| TYR | J03581 | Homo sapiens | tyrosinase(TYR) |
| TYR | AB775901 | Homo sapiens | tyrosinase(TYR) |
| TYR | XM_011542970 | Homo sapiens | tyrosinase(TYR) |
| TYR | AB775899 | Homo sapiens | tyrosinase(TYR) |
| TYR | M27160 | Homo sapiens | tyrosinase(TYR) |
| TYR | AB775900 | Homo sapiens | tyrosinase(TYR) |
| TYR | M74314 | Homo sapiens | tyrosinase(TYR) |
| TYR | BC027179 | Homo sapiens | tyrosinase(TYR) |
| TYR | BU736025 | Homo sapiens | tyrosinase(TYR) |
| TYR | S66645 | Homo sapiens | tyrosinase(TYR) |
| TYR | NM_000372 | Homo sapiens | tyrosinase(TYR) |
| TYR | U01873 | Homo sapiens | tyrosinase(TYR) |
| TYR | Y00819 | Homo sapiens | tyrosinase(TYR) |
| LRP1B | AF176832 | Homo sapiens | LDL receptor related protein 1B(LRP1B) |
| LRP1B | AK054663 | Homo sapiens | LDL receptor related protein 1B(LRP1B) |
| LRP1B | NM_018557 | Homo sapiens | LDL receptor related protein 1B(LRP1B) |
| LRP1B | AB209707 | Homo sapiens | LDL receptor related protein 1B(LRP1B) |
| LRP1B | XR_001738778 | Homo sapiens | LDL receptor related protein 1B(LRP1B) |
| LRP1B | XM_017004341 | Homo sapiens | LDL receptor related protein 1B(LRP1B) |
| LRP1B | XM_017004342 | Homo sapiens | LDL receptor related protein 1B(LRP1B) |
| PHOX2B | NM_003924 | Homo sapiens | paired like homeobox 2b(PHOX2B) |
| PHOX2B | D82344 | Homo sapiens | paired like homeobox 2b(PHOX2B) |
| PHOX2B | AI266171 | Homo sapiens | paired like homeobox 2b(PHOX2B) |
| PHOX2B | BC017199 | Homo sapiens | paired like homeobox 2b(PHOX2B) |
| GFAP | BC041765 | Homo sapiens | glial fibrillary acidic protein(GFAP) |
| GFAP | DA254392 | Homo sapiens | glial fibrillary acidic protein(GFAP) |
| GFAP | BC013596 | Homo sapiens | glial fibrillary acidic protein(GFAP) |
| GFAP | NM_001242376 | Homo sapiens | glial fibrillary acidic protein(GFAP) |
| GFAP | AL133013 | Homo sapiens | glial fibrillary acidic protein(GFAP) |
| GFAP | BC012228 | Homo sapiens | glial fibrillary acidic protein(GFAP) |
| GFAP | AK098758 | Homo sapiens | glial fibrillary acidic protein(GFAP) |
| GFAP | AK303728 | Homo sapiens | glial fibrillary acidic protein(GFAP) |
| GFAP | JF432461 | Homo sapiens | glial fibrillary acidic protein(GFAP) |
| GFAP | M26638 | Homo sapiens | glial fibrillary acidic protein(GFAP) |
| GFAP | NM_001131019 | Homo sapiens | glial fibrillary acidic protein(GFAP) |
| GFAP | AK295734 | Homo sapiens | glial fibrillary acidic protein(GFAP) |
| GFAP | BC127871 | Homo sapiens | glial fibrillary acidic protein(GFAP) |
| GFAP | AK124465 | Homo sapiens | glial fibrillary acidic protein(GFAP) |
| GFAP | BC062609 | Homo sapiens | glial fibrillary acidic protein(GFAP) |
| GFAP | BM931499 | Homo sapiens | glial fibrillary acidic protein(GFAP) |
| GFAP | AK098380 | Homo sapiens | glial fibrillary acidic protein(GFAP) |
| GFAP | AK222683 | Homo sapiens | glial fibrillary acidic protein(GFAP) |
| GFAP | S40719 | Homo sapiens | glial fibrillary acidic protein(GFAP) |
| GFAP | DQ979832 | Homo sapiens | glial fibrillary acidic protein(GFAP) |
| GFAP | XM_017024451 | Homo sapiens | glial fibrillary acidic protein(GFAP) |
| GFAP | J04569 | Homo sapiens | glial fibrillary acidic protein(GFAP) |
| GFAP | U92979 | Homo sapiens | glial fibrillary acidic protein(GFAP) |
| GFAP | BF526347 | Homo sapiens | glial fibrillary acidic protein(GFAP) |
| GFAP | BM687259 | Homo sapiens | glial fibrillary acidic protein(GFAP) |
| GFAP | AF419299 | Homo sapiens | glial fibrillary acidic protein(GFAP) |
| GFAP | BF341765 | Homo sapiens | glial fibrillary acidic protein(GFAP) |
| GFAP | NM_002055 | Homo sapiens | glial fibrillary acidic protein(GFAP) |
| GFAP | AK315398 | Homo sapiens | glial fibrillary acidic protein(GFAP) |
| GFAP | DA315956 | Homo sapiens | glial fibrillary acidic protein(GFAP) |
| GFAP | AJ306447 | Homo sapiens | glial fibrillary acidic protein(GFAP) |
| GFAP | AK128790 | Homo sapiens | glial fibrillary acidic protein(GFAP) |
| GFAP | AK296259 | Homo sapiens | glial fibrillary acidic protein(GFAP) |
| PHOX2A | BQ890461 | Homo sapiens | paired like homeobox 2a(PHOX2A) |
| PHOX2A | AK290645 | Homo sapiens | paired like homeobox 2a(PHOX2A) |
| PHOX2A | NM_005169 | Homo sapiens | paired like homeobox 2a(PHOX2A) |
| PHOX2A | BC041564 | Homo sapiens | paired like homeobox 2a(PHOX2A) |
| PHOX2A | EU446462 | Homo sapiens | paired like homeobox 2a(PHOX2A) |
| GDF7 | BC160118 | Homo sapiens | growth differentiation factor 7(GDF7) |
| GDF7 | BQ182131 | Homo sapiens | growth differentiation factor 7(GDF7) |
| GDF7 | AB158468 | Homo sapiens | growth differentiation factor 7(GDF7) |
| GDF7 | NM_182828 | Homo sapiens | growth differentiation factor 7(GDF7) |
| GDF7 | AF522369 | Homo sapiens | growth differentiation factor 7(GDF7) |
| COL1A1 | AB209597 | Homo sapiens | collagen type I alpha 1 chain(COL1A1) |
| COL1A1 | AK297731 | Homo sapiens | collagen type I alpha 1 chain(COL1A1) |
| COL1A1 | M32798 | Homo sapiens | collagen type I alpha 1 chain(COL1A1) |
| COL1A1 | DQ590066 | Homo sapiens | collagen type I alpha 1 chain(COL1A1) |
| COL1A1 | XM_005257059 | Homo sapiens | collagen type I alpha 1 chain(COL1A1) |
| COL1A1 | XM_005257058 | Homo sapiens | collagen type I alpha 1 chain(COL1A1) |
| COL1A1 | NM_000088 | Homo sapiens | collagen type I alpha 1 chain(COL1A1) |
| COL1A1 | S67495 | Homo sapiens | collagen type I alpha 1 chain(COL1A1) |
| COL1A1 | M36546 | Homo sapiens | collagen type I alpha 1 chain(COL1A1) |
| COL1A1 | JQ236861 | Homo sapiens | collagen type I alpha 1 chain(COL1A1) |
| COL1A1 | Z74615 | Homo sapiens | collagen type I alpha 1 chain(COL1A1) |
| COL1A1 | S64596 | Homo sapiens | collagen type I alpha 1 chain(COL1A1) |
| COL1A1 | EU176569 | Homo sapiens | collagen type I alpha 1 chain(COL1A1) |
| COL1A1 | DQ893571 | Homo sapiens | collagen type I alpha 1 chain(COL1A1) |
| COL1A1 | K01228 | Homo sapiens | collagen type I alpha 1 chain(COL1A1) |
| COL1A1 | XM_011524341 | Homo sapiens | collagen type I alpha 1 chain(COL1A1) |
| COL1A1 | X07884 | Homo sapiens | collagen type I alpha 1 chain(COL1A1) |
| COL1A1 | BC036531 | Homo sapiens | collagen type I alpha 1 chain(COL1A1) |
| COL1A1 | S64717 | Homo sapiens | collagen type I alpha 1 chain(COL1A1) |
| COL1A1 | X06269 | Homo sapiens | collagen type I alpha 1 chain(COL1A1) |
| COL1A2 | AK297786 | Homo sapiens | collagen type I alpha 2 chain(COL1A2) |
| COL1A2 | AK309504 | Homo sapiens | collagen type I alpha 2 chain(COL1A2) |
| COL1A2 | AK300194 | Homo sapiens | collagen type I alpha 2 chain(COL1A2) |
| COL1A2 | NM_000089 | Homo sapiens | collagen type I alpha 2 chain(COL1A2) |
| COL1A2 | AA457209 | Homo sapiens | collagen type I alpha 2 chain(COL1A2) |
| COL1A2 | J03464 | Homo sapiens | collagen type I alpha 2 chain(COL1A2) |
| COL1A2 | AK226074 | Homo sapiens | collagen type I alpha 2 chain(COL1A2) |
| COL1A2 | BC042586 | Homo sapiens | collagen type I alpha 2 chain(COL1A2) |
| COL1A2 | J00114 | Homo sapiens | collagen type I alpha 2 chain(COL1A2) |
| COL1A2 | M22816 | Homo sapiens | collagen type I alpha 2 chain(COL1A2) |
| COL1A2 | X55525 | Homo sapiens | collagen type I alpha 2 chain(COL1A2) |
| COL1A2 | L47668 | Homo sapiens | collagen type I alpha 2 chain(COL1A2) |
| COL1A2 | AL833478 | Homo sapiens | collagen type I alpha 2 chain(COL1A2) |
| COL1A2 | S41099 | Homo sapiens | collagen type I alpha 2 chain(COL1A2) |
| COL1A2 | Z74616 | Homo sapiens | collagen type I alpha 2 chain(COL1A2) |
| COL1A2 | S62614 | Homo sapiens | collagen type I alpha 2 chain(COL1A2) |
| COL1A2 | S96821 | Homo sapiens | collagen type I alpha 2 chain(COL1A2) |
| COL1A2 | V00503 | Homo sapiens | collagen type I alpha 2 chain(COL1A2) |
| COL1A2 | KU177988 | Homo sapiens | collagen type I alpha 2 chain(COL1A2) |
| COL1A2 | Y00724 | Homo sapiens | collagen type I alpha 2 chain(COL1A2) |
| COL1A2 | KU177989 | Homo sapiens | collagen type I alpha 2 chain(COL1A2) |
| COL1A2 | CF125721 | Homo sapiens | collagen type I alpha 2 chain(COL1A2) |
| COL1A2 | X02488 | Homo sapiens | collagen type I alpha 2 chain(COL1A2) |
| COL1A2 | BC054498 | Homo sapiens | collagen type I alpha 2 chain(COL1A2) |
| KIF4A | AB208797 | Homo sapiens | kinesin family member 4A(KIF4A) |
| KIF4A | BC038459 | Homo sapiens | kinesin family member 4A(KIF4A) |
| KIF4A | AK022592 | Homo sapiens | kinesin family member 4A(KIF4A) |
| KIF4A | AW592235 | Homo sapiens | kinesin family member 4A(KIF4A) |
| KIF4A | BC049218 | Homo sapiens | kinesin family member 4A(KIF4A) |
| KIF4A | AK024266 | Homo sapiens | kinesin family member 4A(KIF4A) |
| KIF4A | BC011801 | Homo sapiens | kinesin family member 4A(KIF4A) |
| KIF4A | AF071592 | Homo sapiens | kinesin family member 4A(KIF4A) |
| KIF4A | AF179308 | Homo sapiens | kinesin family member 4A(KIF4A) |
| KIF4A | AK000638 | Homo sapiens | kinesin family member 4A(KIF4A) |
| KIF4A | BC003664 | Homo sapiens | kinesin family member 4A(KIF4A) |
| KIF4A | BC050548 | Homo sapiens | kinesin family member 4A(KIF4A) |
| KIF4A | AF277375 | Homo sapiens | kinesin family member 4A(KIF4A) |
| KIF4A | AK022717 | Homo sapiens | kinesin family member 4A(KIF4A) |
| KIF4A | AK313133 | Homo sapiens | kinesin family member 4A(KIF4A) |
| KIF4A | NM_012310 | Homo sapiens | kinesin family member 4A(KIF4A) |
| KIF4A | AJ271784 | Homo sapiens | kinesin family member 4A(KIF4A) |
| KIF4A | DA021880 | Homo sapiens | kinesin family member 4A(KIF4A) |
| COL5A2 | M10956 | Homo sapiens | collagen type V alpha 2 chain(COL5A2) |
| COL5A2 | J04478 | Homo sapiens | collagen type V alpha 2 chain(COL5A2) |
| COL5A2 | BC043613 | Homo sapiens | collagen type V alpha 2 chain(COL5A2) |
| COL5A2 | M11135 | Homo sapiens | collagen type V alpha 2 chain(COL5A2) |
| COL5A2 | BC015705 | Homo sapiens | collagen type V alpha 2 chain(COL5A2) |
| COL5A2 | AK300146 | Homo sapiens | collagen type V alpha 2 chain(COL5A2) |
| COL5A2 | AL552427 | Homo sapiens | collagen type V alpha 2 chain(COL5A2) |
| COL5A2 | M31365 | Homo sapiens | collagen type V alpha 2 chain(COL5A2) |
| COL5A2 | AB209045 | Homo sapiens | collagen type V alpha 2 chain(COL5A2) |
| COL5A2 | BC086874 | Homo sapiens | collagen type V alpha 2 chain(COL5A2) |
| COL5A2 | XM_011510573 | Homo sapiens | collagen type V alpha 2 chain(COL5A2) |
| COL5A2 | AK297936 | Homo sapiens | collagen type V alpha 2 chain(COL5A2) |
| COL5A2 | X04758 | Homo sapiens | collagen type V alpha 2 chain(COL5A2) |
| COL5A2 | NM_000393 | Homo sapiens | collagen type V alpha 2 chain(COL5A2) |
| COL5A2 | Y14690 | Homo sapiens | collagen type V alpha 2 chain(COL5A2) |
| COL5A2 | M11718 | Homo sapiens | collagen type V alpha 2 chain(COL5A2) |
| FAT4 | NM_001291285 | Homo sapiens | FAT atypical cadherin 4(FAT4) |
| FAT4 | AK291461 | Homo sapiens | FAT atypical cadherin 4(FAT4) |
| FAT4 | AL713715 | Homo sapiens | FAT atypical cadherin 4(FAT4) |
| FAT4 | KJ131484 | Homo sapiens | FAT atypical cadherin 4(FAT4) |
| FAT4 | KJ131485 | Homo sapiens | FAT atypical cadherin 4(FAT4) |
| FAT4 | DT932755 | Homo sapiens | FAT atypical cadherin 4(FAT4) |
| FAT4 | KJ131486 | Homo sapiens | FAT atypical cadherin 4(FAT4) |
| FAT4 | KJ131487 | Homo sapiens | FAT atypical cadherin 4(FAT4) |
| FAT4 | NM_001291303 | Homo sapiens | FAT atypical cadherin 4(FAT4) |
| FAT4 | KJ131488 | Homo sapiens | FAT atypical cadherin 4(FAT4) |
| FAT4 | AK091292 | Homo sapiens | FAT atypical cadherin 4(FAT4) |
| FAT4 | AK096646 | Homo sapiens | FAT atypical cadherin 4(FAT4) |
| FAT4 | AK026709 | Homo sapiens | FAT atypical cadherin 4(FAT4) |
| FAT4 | DT932756 | Homo sapiens | FAT atypical cadherin 4(FAT4) |
| FAT4 | KJ131481 | Homo sapiens | FAT atypical cadherin 4(FAT4) |
| FAT4 | XM_011532236 | Homo sapiens | FAT atypical cadherin 4(FAT4) |
| FAT4 | KJ131482 | Homo sapiens | FAT atypical cadherin 4(FAT4) |
| FAT4 | KJ131483 | Homo sapiens | FAT atypical cadherin 4(FAT4) |
| FAT4 | AY356402 | Homo sapiens | FAT atypical cadherin 4(FAT4) |
| FAT4 | NM_024582 | Homo sapiens | FAT atypical cadherin 4(FAT4) |
| FAT4 | XM_011532237 | Homo sapiens | FAT atypical cadherin 4(FAT4) |
| FAT4 | AB075518 | Homo sapiens | FAT atypical cadherin 4(FAT4) |
| CD24 | BU580553 | Homo sapiens | CD24 molecule(CD24) |
| CD24 | DA753855 | Homo sapiens | CD24 molecule(CD24) |
| CD24 | DQ890636 | Homo sapiens | CD24 molecule(CD24) |
| CD24 | NR_117089 | Homo sapiens | CD24 molecule(CD24) |
| CD24 | AU125939 | Homo sapiens | CD24 molecule(CD24) |
| CD24 | NM_013230 | Homo sapiens | CD24 molecule(CD24) |
| CD24 | BT007404 | Homo sapiens | CD24 molecule(CD24) |
| CD24 | AK026603 | Homo sapiens | CD24 molecule(CD24) |
| CD24 | DA745135 | Homo sapiens | CD24 molecule(CD24) |
| CD24 | NM_001291739 | Homo sapiens | CD24 molecule(CD24) |
| CD24 | NM_001291737 | Homo sapiens | CD24 molecule(CD24) |
| CD24 | NM_001291738 | Homo sapiens | CD24 molecule(CD24) |
| CD24 | AL535013 | Homo sapiens | CD24 molecule(CD24) |
| CD24 | AK125531 | Homo sapiens | CD24 molecule(CD24) |
| CD24 | DQ893812 | Homo sapiens | CD24 molecule(CD24) |
| CD24 | X69397 | Homo sapiens | CD24 molecule(CD24) |
| CD24 | D87667 | Homo sapiens | CD24 molecule(CD24) |
| CD24 | M58664 | Homo sapiens | CD24 molecule(CD24) |
| CD24 | BG755979 | Homo sapiens | CD24 molecule(CD24) |
| CD24 | DQ530234 | Homo sapiens | CD24 molecule(CD24) |
| CD24 | DA378105 | Homo sapiens | CD24 molecule(CD24) |
| CD24 | DQ530232 | Homo sapiens | CD24 molecule(CD24) |
| CD24 | DQ530233 | Homo sapiens | CD24 molecule(CD24) |
| CD24 | DQ530230 | Homo sapiens | CD24 molecule(CD24) |
| CD24 | DQ530231 | Homo sapiens | CD24 molecule(CD24) |
| CD24 | BC064619 | Homo sapiens | CD24 molecule(CD24) |
| CD24 | BG260536 | Homo sapiens | CD24 molecule(CD24) |
| CD24 | NR_117090 | Homo sapiens | CD24 molecule(CD24) |
| CD24 | L33930 | Homo sapiens | CD24 molecule(CD24) |
| CD24 | DA518957 | Homo sapiens | CD24 molecule(CD24) |
| CD24 | DA253943 | Homo sapiens | CD24 molecule(CD24) |
| MXD3 | AF114834 | Homo sapiens | MAX dimerization protein 3(MXD3) |
| MXD3 | AK057034 | Homo sapiens | MAX dimerization protein 3(MXD3) |
| MXD3 | AK222579 | Homo sapiens | MAX dimerization protein 3(MXD3) |
| MXD3 | AK303397 | Homo sapiens | MAX dimerization protein 3(MXD3) |
| MXD3 | DQ892734 | Homo sapiens | MAX dimerization protein 3(MXD3) |
| MXD3 | NM_001142935 | Homo sapiens | MAX dimerization protein 3(MXD3) |
| MXD3 | BC041690 | Homo sapiens | MAX dimerization protein 3(MXD3) |
| MXD3 | AL833959 | Homo sapiens | MAX dimerization protein 3(MXD3) |
| MXD3 | BM849150 | Homo sapiens | MAX dimerization protein 3(MXD3) |
| MXD3 | BU620484 | Homo sapiens | MAX dimerization protein 3(MXD3) |
| MXD3 | AK092842 | Homo sapiens | MAX dimerization protein 3(MXD3) |
| MXD3 | NM_031300 | Homo sapiens | MAX dimerization protein 3(MXD3) |
| MXD3 | BC032586 | Homo sapiens | MAX dimerization protein 3(MXD3) |
| MXD3 | DQ896213 | Homo sapiens | MAX dimerization protein 3(MXD3) |
| MXD3 | AK316468 | Homo sapiens | MAX dimerization protein 3(MXD3) |
| MXD3 | BC000745 | Homo sapiens | MAX dimerization protein 3(MXD3) |
| MXD3 | AL522759 | Homo sapiens | MAX dimerization protein 3(MXD3) |
| MXD3 | DQ895981 | Homo sapiens | MAX dimerization protein 3(MXD3) |
| RNF220 | BC062600 | Homo sapiens | ring finger protein 220(RNF220) |
| RNF220 | CN285725 | Homo sapiens | ring finger protein 220(RNF220) |
| RNF220 | DC340836 | Homo sapiens | ring finger protein 220(RNF220) |
| RNF220 | XR_946692 | Homo sapiens | ring finger protein 220(RNF220) |
| RNF220 | XM_011541700 | Homo sapiens | ring finger protein 220(RNF220) |
| RNF220 | XM_011541702 | Homo sapiens | ring finger protein 220(RNF220) |
| RNF220 | NM_001319956 | Homo sapiens | ring finger protein 220(RNF220) |
| RNF220 | NM_001319957 | Homo sapiens | ring finger protein 220(RNF220) |
| RNF220 | XM_005270993 | Homo sapiens | ring finger protein 220(RNF220) |
| RNF220 | XM_017001625 | Homo sapiens | ring finger protein 220(RNF220) |
| RNF220 | XM_005270992 | Homo sapiens | ring finger protein 220(RNF220) |
| RNF220 | XM_017001626 | Homo sapiens | ring finger protein 220(RNF220) |
| RNF220 | XM_005270996 | Homo sapiens | ring finger protein 220(RNF220) |
| RNF220 | XM_017001623 | Homo sapiens | ring finger protein 220(RNF220) |
| RNF220 | BC000279 | Homo sapiens | ring finger protein 220(RNF220) |
| RNF220 | XM_017001624 | Homo sapiens | ring finger protein 220(RNF220) |
| RNF220 | BC073835 | Homo sapiens | ring finger protein 220(RNF220) |
| RNF220 | AF151080 | Homo sapiens | ring finger protein 220(RNF220) |
| RNF220 | AK293411 | Homo sapiens | ring finger protein 220(RNF220) |
| RNF220 | BC034221 | Homo sapiens | ring finger protein 220(RNF220) |
| RNF220 | AK056424 | Homo sapiens | ring finger protein 220(RNF220) |
| RNF220 | XR_001737281 | Homo sapiens | ring finger protein 220(RNF220) |
| RNF220 | XM_011541698 | Homo sapiens | ring finger protein 220(RNF220) |
| RNF220 | XM_011541699 | Homo sapiens | ring finger protein 220(RNF220) |
| RNF220 | NM_018150 | Homo sapiens | ring finger protein 220(RNF220) |
| RNF220 | AK025744 | Homo sapiens | ring finger protein 220(RNF220) |
| RNF220 | XR_001737280 | Homo sapiens | ring finger protein 220(RNF220) |
| RNF220 | BC098300 | Homo sapiens | ring finger protein 220(RNF220) |
| RNF220 | BC105790 | Homo sapiens | ring finger protein 220(RNF220) |
| RNF220 | XR_001737278 | Homo sapiens | ring finger protein 220(RNF220) |
| RNF220 | BC098266 | Homo sapiens | ring finger protein 220(RNF220) |
| RNF220 | XR_001737279 | Homo sapiens | ring finger protein 220(RNF220) |
| RNF220 | AK001459 | Homo sapiens | ring finger protein 220(RNF220) |
| RNF220 | XM_006710735 | Homo sapiens | ring finger protein 220(RNF220) |
| RNF220 | AK124790 | Homo sapiens | ring finger protein 220(RNF220) |
| RNF220 | AK225180 | Homo sapiens | ring finger protein 220(RNF220) |
| RNF220 | AK297228 | Homo sapiens | ring finger protein 220(RNF220) |
| RNF220 | AK296038 | Homo sapiens | ring finger protein 220(RNF220) |
| SNAP25 | L19760 | Homo sapiens | synaptosome associated protein 25(SNAP25) |
| SNAP25 | XM_017028021 | Homo sapiens | synaptosome associated protein 25(SNAP25) |
| SNAP25 | XM_017028022 | Homo sapiens | synaptosome associated protein 25(SNAP25) |
| SNAP25 | L19761 | Homo sapiens | synaptosome associated protein 25(SNAP25) |
| SNAP25 | XM_017028023 | Homo sapiens | synaptosome associated protein 25(SNAP25) |
| SNAP25 | HY127744 | Homo sapiens | synaptosome associated protein 25(SNAP25) |
| SNAP25 | NM_001322909 | Homo sapiens | synaptosome associated protein 25(SNAP25) |
| SNAP25 | DA072367 | Homo sapiens | synaptosome associated protein 25(SNAP25) |
| SNAP25 | XM_005260808 | Homo sapiens | synaptosome associated protein 25(SNAP25) |
| SNAP25 | BC010647 | Homo sapiens | synaptosome associated protein 25(SNAP25) |
| SNAP25 | NM_130811 | Homo sapiens | synaptosome associated protein 25(SNAP25) |
| SNAP25 | AK314359 | Homo sapiens | synaptosome associated protein 25(SNAP25) |
| SNAP25 | AK090857 | Homo sapiens | synaptosome associated protein 25(SNAP25) |
| SNAP25 | NM_001322910 | Homo sapiens | synaptosome associated protein 25(SNAP25) |
| SNAP25 | AM393230 | Homo sapiens | synaptosome associated protein 25(SNAP25) |
| SNAP25 | BT019684 | Homo sapiens | synaptosome associated protein 25(SNAP25) |
| SNAP25 | HQ447952 | Homo sapiens | synaptosome associated protein 25(SNAP25) |
| SNAP25 | BM666216 | Homo sapiens | synaptosome associated protein 25(SNAP25) |
| SNAP25 | AK094560 | Homo sapiens | synaptosome associated protein 25(SNAP25) |
| SNAP25 | BI604120 | Homo sapiens | synaptosome associated protein 25(SNAP25) |
| SNAP25 | BI552777 | Homo sapiens | synaptosome associated protein 25(SNAP25) |
| SNAP25 | BI602062 | Homo sapiens | synaptosome associated protein 25(SNAP25) |
| SNAP25 | AK026732 | Homo sapiens | synaptosome associated protein 25(SNAP25) |
| SNAP25 | NM_001322902 | Homo sapiens | synaptosome associated protein 25(SNAP25) |
| SNAP25 | AK289647 | Homo sapiens | synaptosome associated protein 25(SNAP25) |
| SNAP25 | D21267 | Homo sapiens | synaptosome associated protein 25(SNAP25) |
| SNAP25 | DA162693 | Homo sapiens | synaptosome associated protein 25(SNAP25) |
| SNAP25 | NM_001322904 | Homo sapiens | synaptosome associated protein 25(SNAP25) |
| SNAP25 | NM_001322903 | Homo sapiens | synaptosome associated protein 25(SNAP25) |
| SNAP25 | AK098347 | Homo sapiens | synaptosome associated protein 25(SNAP25) |
| SNAP25 | NM_001322906 | Homo sapiens | synaptosome associated protein 25(SNAP25) |
| SNAP25 | NM_001322905 | Homo sapiens | synaptosome associated protein 25(SNAP25) |
| SNAP25 | AK098767 | Homo sapiens | synaptosome associated protein 25(SNAP25) |
| SNAP25 | NM_001322908 | Homo sapiens | synaptosome associated protein 25(SNAP25) |
| SNAP25 | NM_001322907 | Homo sapiens | synaptosome associated protein 25(SNAP25) |
| SNAP25 | AK223617 | Homo sapiens | synaptosome associated protein 25(SNAP25) |
| SNAP25 | AM393653 | Homo sapiens | synaptosome associated protein 25(SNAP25) |
| SNAP25 | BX647443 | Homo sapiens | synaptosome associated protein 25(SNAP25) |
| SNAP25 | NM_003081 | Homo sapiens | synaptosome associated protein 25(SNAP25) |
| FOXM1 | NM_001243088 | Homo sapiens | forkhead box M1(FOXM1) |
| FOXM1 | NM_001243089 | Homo sapiens | forkhead box M1(FOXM1) |
| FOXM1 | AY542306 | Homo sapiens | forkhead box M1(FOXM1) |
| FOXM1 | BC012863 | Homo sapiens | forkhead box M1(FOXM1) |
| FOXM1 | NM_021953 | Homo sapiens | forkhead box M1(FOXM1) |
| FOXM1 | U74613 | Homo sapiens | forkhead box M1(FOXM1) |
| FOXM1 | U74612 | Homo sapiens | forkhead box M1(FOXM1) |
| FOXM1 | AW780338 | Homo sapiens | forkhead box M1(FOXM1) |
| FOXM1 | BC035437 | Homo sapiens | forkhead box M1(FOXM1) |
| FOXM1 | XM_005253676 | Homo sapiens | forkhead box M1(FOXM1) |
| FOXM1 | BQ229497 | Homo sapiens | forkhead box M1(FOXM1) |
| FOXM1 | L16783 | Homo sapiens | forkhead box M1(FOXM1) |
| FOXM1 | AK313845 | Homo sapiens | forkhead box M1(FOXM1) |
| FOXMI | BC006192 | Homo sapiens | forkhead box M1(FOXM1) |
| FOXM1 | XM_011520932 | Homo sapiens | forkhead box M1(FOXM1) |
| FOXM1 | XM_011520931 | Homo sapiens | forkhead box M1(FOXM1) |
| FOXM1 | XM_011520930 | Homo sapiens | forkhead box M1(FOXM1) |
| FOXM1 | XR_931507 | Homo sapiens | forkhead box M1(FOXM1) |
| FOXM1 | BT006986 | Homo sapiens | forkhead box M1(FOXM1) |
| FOXM1 | NM_202002 | Homo sapiens | forkhead box M1(FOXM1) |
| FOXM1 | NM_202003 | Homo sapiens | forkhead box M1(FOXM1) |
| FOXM1 | XM_011520935 | Homo sapiens | forkhead box M1(FOXM1) |
| FOXM1 | XM_011520934 | Homo sapiens | forkhead box M1(FOXM1) |
| FOXM1 | XM_011520933 | Homo sapiens | forkhead box M1(FOXM1) |
| FOXM1 | BC006529 | Homo sapiens | forkhead box M1(FOXM1) |
| FOXM1 | DB137846 | Homo sapiens | forkhead box M1(FOXM1) |
| FOXM1 | AK291206 | Homo sapiens | forkhead box M1(FOXM1) |
| FOXM1 | JF432495 | Homo sapiens | forkhead box M1(FOXM1) |
| FOXM1 | U83113 | Homo sapiens | forkhead box M1(FOXM1) |
| CHD1 | AF006513 | Homo sapiens | chromodomain helicase DNA binding protein 1(CHD1) |
| CHD1 | BC117134 | Homo sapiens | chromodomain helicase DNA binding protein 1(CHD1) |
| CHD1 | XM_011543112 | Homo sapiens | chromodomain helicase DNA binding protein 1(CHD1) |
| CHD1 | CN277742 | Homo sapiens | chromodomain helicase DNA binding protein 1(CHD1) |
| CHD1 | XR_427702 | Homo sapiens | chromodomain helicase DNA binding protein 1(CHD1) |
| CHD1 | XM_017008991 | Homo sapiens | chromodomain helicase DNA binding protein 1(CHD1) |
| CHD1 | BQ436172 | Homo sapiens | chromodomain helicase DNA binding protein 1(CHD1) |
| CHD1 | XM_017008992 | Homo sapiens | chromodomain helicase DNA binding protein 1(CHD1) |
| CHD1 | XR_001741986 | Homo sapiens | chromodomain helicase DNA binding protein 1(CHD1) |
| CHD1 | NM_001270 | Homo sapiens | chromodomain helicase DNA binding protein 1(CHD1) |
| CHD1 | XM_005271866 | Homo sapiens | chromodomain helicase DNA binding protein 1(CHD1) |
| CHD1 | AK094871 | Homo sapiens | chromodomain helicase DNA binding protein 1(CHD1) |
| CHD1 | BX648767 | Homo sapiens | chromodomain helicase DNA binding protein 1(CHD1) |
| CHD1 | XM_005271867 | Homo sapiens | chromodomain helicase DNA binding protein 1(CHD1) |
| CHD1 | BM698633 | Homo sapiens | chromodomain helicase DNA binding protein 1(CHD1) |
| CHD1 | BC054860 | Homo sapiens | chromodomain helicase DNA binding protein 1(CHD1) |
| ELAVL2 | XM_017014418 | Homo sapiens | ELAV like RNA binding protein 2(ELAVL2) |
| ELAVL2 | XM_017014419 | Homo sapiens | ELAV like RNA binding protein 2(ELAVL2) |
| ELAVL2 | XR_929211 | Homo sapiens | ELAV like RNA binding protein 2(ELAVL2) |
| ELAVL2 | XM_017014414 | Homo sapiens | ELAV like RNA binding protein 2(ELAVL2) |
| ELAVL2 | XM_017014415 | Homo sapiens | ELAV like RNA binding protein 2(ELAVL2) |
| ELAVL2 | XM_017014416 | Homo sapiens | ELAV like RNA binding protein 2(ELAVL2) |
| ELAVL2 | XM_011517774 | Homo sapiens | ELAV like RNA binding protein 2(ELAVL2) |
| ELAVL2 | XM_017014417 | Homo sapiens | ELAV like RNA binding protein 2(ELAVL2) |
| ELAVL2 | AB209294 | Homo sapiens | ELAV like RNA binding protein 2(ELAVL2) |
| ELAVL2 | BC042393 | Homo sapiens | ELAV like RNA binding protein 2(ELAVL2) |
| ELAVL2 | XM_005251395 | Homo sapiens | ELAV like RNA binding protein 2(ELAVL2) |
| ELAVL2 | XM_005251394 | Homo sapiens | ELAV like RNA binding protein 2(ELAVL2) |
| ELAVL2 | XM_005251393 | Homo sapiens | ELAV like RNA binding protein 2(ELAVL2) |
| ELAVL2 | XR_929212 | Homo sapiens | ELAV like RNA binding protein 2(ELAVL2) |
| ELAVL2 | NM_001171197 | Homo sapiens | ELAV like RNA binding protein 2(ELAVL2) |
| ELAVL2 | NM_001171195 | Homo sapiens | ELAV like RNA binding protein 2(ELAVL2) |
| ELAVL2 | XM_011517786 | Homo sapiens | ELAV like RNA binding protein 2(ELAVL2) |
| ELAVL2 | XM_017014410 | Homo sapiens | ELAV like RNA binding protein 2(ELAVL2) |
| ELAVL2 | XM_017014411 | Homo sapiens | ELAV like RNA binding protein 2(ELAVL2) |
| ELAVL2 | XM_017014412 | Homo sapiens | ELAV like RNA binding protein 2(ELAVL2) |
| ELAVL2 | XM_017014413 | Homo sapiens | ELAV like RNA binding protein 2(ELAVL2) |
| ELAVL2 | XM_017014408 | Homo sapiens | ELAV like RNA binding protein 2(ELAVL2) |
| ELAVL2 | XM_011517780 | Homo sapiens | ELAV like RNA binding protein 2(ELAVL2) |
| ELAVL2 | XM_017014409 | Homo sapiens | ELAV like RNA binding protein 2(ELAVL2) |
| ELAVL2 | XM_017014425 | Homo sapiens | ELAV like RNA binding protein 2(ELAVL2) |
| ELAVL2 | XM_011517783 | Homo sapiens | ELAV like RNA binding protein 2(ELAVL2) |
| ELAVL2 | XM_017014426 | Homo sapiens | ELAV like RNA binding protein 2(ELAVL2) |
| ELAVL2 | XM_011517784 | Homo sapiens | ELAV like RNA binding protein 2(ELAVL2) |
| ELAVL2 | XM_011517785 | Homo sapiens | ELAV like RNA binding protein 2(ELAVL2) |
| ELAVL2 | DQ894809 | Homo sapiens | ELAV like RNA binding protein 2(ELAVL2) |
| ELAVL2 | DQ891617 | Homo sapiens | ELAV like RNA binding protein 2(ELAVL2) |
| ELAVL2 | U13706 | Homo sapiens | ELAV like RNA binding protein 2(ELAVL2) |
| ELAVL2 | XM_006716736 | Homo sapiens | ELAV like RNA binding protein 2(ELAVL2) |
| ELAVL2 | NM_004432 | Homo sapiens | ELAV like RNA binding protein 2(ELAVL2) |
| ELAVL2 | XM_006716735 | Homo sapiens | ELAV like RNA binding protein 2(ELAVL2) |
| ELAVL2 | XM_006716734 | Homo sapiens | ELAV like RNA binding protein 2(ELAVL2) |
| ELAVL2 | XM_017014421 | Homo sapiens | ELAV like RNA binding protein 2(ELAVL2) |
| ELAVL2 | EU176647 | Homo sapiens | ELAV like RNA binding protein 2(ELAVL2) |
| ELAVL2 | XM_011517776 | Homo sapiens | ELAV like RNA binding protein 2(ELAVL2) |
| ELAVL2 | XM_017014422 | Homo sapiens | ELAV like RNA binding protein 2(ELAVL2) |
| ELAVL2 | XM_011517777 | Homo sapiens | ELAV like RNA binding protein 2(ELAVL2) |
| ELAVL2 | XM_017014423 | Homo sapiens | ELAV like RNA binding protein 2(ELAVL2) |
| ELAVL2 | XM_011517778 | Homo sapiens | ELAV like RNA binding protein 2(ELAVL2) |
| ELAVL2 | XM_017014424 | Homo sapiens | ELAV like RNA binding protein 2(ELAVL2) |
| ELAVL2 | BC035004 | Homo sapiens | ELAV like RNA binding protein 2(ELAVL2) |
| ELAVL2 | XM_011517779 | Homo sapiens | ELAV like RNA binding protein 2(ELAVL2) |
| ELAVL2 | BC030692 | Homo sapiens | ELAV like RNA binding protein 2(ELAVL2) |
| ELAVL2 | XM_017014420 | Homo sapiens | ELAV like RNA binding protein 2(ELAVL2) |
| SHH | XM_011516480 | Homo sapiens | sonic hedgehog(SHH) |
| SHH | BX452210 | Homo sapiens | sonic hedgehog(SHH) |
| SHH | NR_132318 | Homo sapiens | sonic hedgehog(SHH) |
| SHH | NR_132319 | Homo sapiens | sonic hedgehog(SHH) |
| SHH | AI192528 | Homo sapiens | sonic hedgehog(SHH) |
| SHH | L38518 | Homo sapiens | sonic hedgehog(SHH) |
| SHH | NM_001310462 | Homo sapiens | sonic hedgehog(SHH) |
| SHH | AY927454 | Homo sapiens | sonic hedgehog(SHH) |
| SHH | AY927453 | Homo sapiens | sonic hedgehog(SHH) |
| SHH | BC111925 | Homo sapiens | sonic hedgehog(SHH) |
| SHH | XM_011516479 | Homo sapiens | sonic hedgehog(SHH) |
| SHH | AY927455 | Homo sapiens | sonic hedgehog(SHH) |
| SHH | AY927450 | Homo sapiens | sonic hedgehog(SHH) |
| SHH | BX109787 | Homo sapiens | sonic hedgehog(SHH) |
| SHH | NM_000193 | Homo sapiens | sonic hedgehog(SHH) |
| SHH | BY797055 | Homo sapiens | sonic hedgehog(SHH) |
| SHH | AY927452 | Homo sapiens | sonic hedgehog(SHH) |
| SHH | AY927451 | Homo sapiens | sonic hedgehog(SHH) |
| EPB41L4A | XM_017009689 | Homo sapiens | erythrocyte membrane protein band 4.1 like 4A(EPB41L4A) |
| EPB41L4A | AL117425 | Homo sapiens | erythrocyte membrane protein band 4.1 like 4A(EPB41L4A) |
| EPB41L4A | XM_011543530 | Homo sapiens | erythrocyte membrane protein band 4.1 like 4A(EPB41L4A) |
| EPB41L4A | XR_001742173 | Homo sapiens | erythrocyte membrane protein band 4.1 like 4A(EPB41L4A) |
| EPB41L4A | AA406206 | Homo sapiens | erythrocyte membrane protein band 4.1 like 4A(EPB41L4A) |
| EPB41L4A | XM_011543531 | Homo sapiens | erythrocyte membrane protein band 4.1 like 4A(EPB41L4A) |
| EPB41L4A | XM_011543532 | Homo sapiens | erythrocyte membrane protein band 4.1 like 4A(EPB41L4A) |
| EPB41L4A | XR_001742175 | Homo sapiens | erythrocyte membrane protein band 4.1 like 4A(EPB41L4A) |
| EPB41L4A | XM_011543533 | Homo sapiens | erythrocyte membrane protein band 4.1 like 4A(EPB41L4A) |
| EPB41L4A | AK096057 | Homo sapiens | erythrocyte membrane protein band 4.1 like 4A(EPB41L4A) |
| EPB41L4A | CB048933 | Homo sapiens | erythrocyte membrane protein band 4.1 like 4A(EPB41L4A) |
| EPB41L4A | AA350575 | Homo sapiens | erythrocyte membrane protein band 4.1 like 4A(EPB41L4A) |
| EPB41L4A | AK021578 | Homo sapiens | erythrocyte membrane protein band 4.1 like 4A(EPB41L4A) |
| EPB41L4A | JF432740 | Homo sapiens | erythrocyte membrane protein band 4.1 like 4A(EPB41L4A) |
| EPB41L4A | AK123285 | Homo sapiens | erythrocyte membrane protein band 4.1 like 4A(EPB41L4A) |
| EPB41L4A | BC114942 | Homo sapiens | erythrocyte membrane protein band 4.1 like 4A(EPB41L4A) |
| EPB41L4A | BC114632 | Homo sapiens | erythrocyte membrane protein band 4.1 like 4A(EPB41L4A) |
| EPB41L4A | AB030240 | Homo sapiens | erythrocyte membrane protein band 4.1 like 4A(EPB41L4A) |
| EPB41L4A | NM_022140 | Homo sapiens | erythrocyte membrane protein band 4.1 like 4A(EPB41L4A) |
| EPB41L4A | BC160044 | Homo sapiens | erythrocyte membrane protein band 4.1 like 4A(EPB41L4A) |
| EPB41L4A | BC031042 | Homo sapiens | erythrocyte membrane protein band 4.1 like 4A(EPB41L4A) |
| EPB41L4A | BC015441 | Homo sapiens | erythrocyte membrane protein band 4.1 like 4A(EPB41L4A) |
| ADAMTS3 | AF247668 | Homo sapiens | ADAM metallopeptidase with thrombospondin type 1 motif 3(ADAMTS3) |
| ADAMTS3 | BC019707 | Homo sapiens | ADAM metallopeptidase with thrombospondin type 1 motif 3(ADAMTS3) |
| ADAMTS3 | XM_011532422 | Homo sapiens | ADAM metallopeptidase with thrombospondin type 1 motif 3(ADAMTS3) |
| ADAMTS3 | AK295131 | Homo sapiens | ADAM metallopeptidase with thrombospondin type 1 motif 3(ADAMTS3) |
| ADAMTS3 | BC130287 | Homo sapiens | ADAM metallopeptidase with thrombospondin type 1 motif 3(ADAMTS3) |
| ADAMTS3 | XM_011532421 | Homo sapiens | ADAM metallopeptidase with thrombospondin type 1 motif 3(ADAMTS3) |
| ADAMTS3 | BC132735 | Homo sapiens | ADAM metallopeptidase with thrombospondin type 1 motif 3(ADAMTS3) |
| ADAMTS3 | NM_014243 | Homo sapiens | ADAM metallopeptidase with thrombospondin type 1 motif 3(ADAMTS3) |
| ADAMTS3 | AB002364 | Homo sapiens | ADAM metallopeptidase with thrombospondin type 1 motif 3(ADAMTS3) |
| ADAMTS3 | BC016451 | Homo sapiens | ADAM metallopeptidase with thrombospondin type 1 motif 3(ADAMTS3) |
| SIX3 | AJ012611 | Homo sapiens | SIX Homeobox 3(SIX3) |
| SIX3 | BF569811 | Homo sapiens | SIX Homeobox 3(SIX3) |
| SIX3 | EL952320 | Homo sapiens | SIX Homeobox 3(SIX3) |
| SIX3 | BF570321 | Homo sapiens | SIX Homeobox 3(SIX3) |
| SIX3 | CD673488 | Homo sapiens | SIX Homeobox 3(SIX3) |
| SIX3 | BC153026 | Homo sapiens | SIX Homeobox 3(SIX3) |
| SIX3 | HG493938 | Homo sapiens | SIX Homeobox 3(SIX3) |
| SIX3 | BC030289 | Homo sapiens | SIX Homeobox 3(SIX3) |
| SIX3 | NM_005413 | Homo sapiens | SIX Homeobox 3(SIX3) |
| SIX3 | AL162671 | Homo sapiens | SIX Homeobox 3(SIX3) |
| SIX3 | BM668514 | Homo sapiens | SIX Homeobox 3(SIX3) |
| GRM8 | BC093725 | Homo sapiens | glutamate metabotropic receptor 8(GRM8) |
| GRM8 | XM_011516102 | Homo sapiens | glutamate metabotropic receptor 8(GRM8) |
| GRM8 | XM_011516101 | Homo sapiens | glutamate metabotropic receptor 8(GRM8) |
| GRM8 | BC101675 | Homo sapiens | glutamate metabotropic receptor 8(GRM8) |
| GRM8 | XM_006715938 | Homo sapiens | glutamate metabotropic receptor 8(GRM8) |
| GRM8 | AK123053 | Homo sapiens | glutamate metabotropic receptor 8(GRM8) |
| GRM8 | AY608335 | Homo sapiens | glutamate metabotropic receptor 8(GRM8) |
| GRM8 | XM_017012077 | Homo sapiens | glutamate metabotropic receptor 8(GRM8) |
| GRM8 | XM_017012076 | Homo sapiens | glutamate metabotropic receptor 8(GRM8) |
| GRM8 | AK315203 | Homo sapiens | glutamate metabotropic receptor 8(GRM8) |
| GRM8 | XM_017012075 | Homo sapiens | glutamate metabotropic receptor 8(GRM8) |
| GRM8 | XM_017012074 | Homo sapiens | glutamate metabotropic receptor 8(GRM8) |
| GRM8 | AJ236922 | Homo sapiens | glutamate metabotropic receptor 8(GRM8) |
| GRM8 | XM_017012079 | Homo sapiens | glutamate metabotropic receptor 8(GRM8) |
| GRM8 | AJ236921 | Homo sapiens | glutamate metabotropic receptor 8(GRM8) |
| GRM8 | XM_017012078 | Homo sapiens | glutamate metabotropic receptor 8(GRM8) |
| GRM8 | XM_011516094 | Homo sapiens | glutamate metabotropic receptor 8(GRM8) |
| GRM8 | XM_011516092 | Homo sapiens | glutamate metabotropic receptor 8(GRM8) |
| GRM8 | XM_011516091 | Homo sapiens | glutamate metabotropic receptor 8(GRM8) |
| GRM8 | BC143344 | Homo sapiens | glutamate metabotropic receptor 8(GRM8) |
| GRM8 | U95025 | Homo sapiens | glutamate metabotropic receptor 8(GRM8) |
| GRM8 | BG211606 | Homo sapiens | glutamate metabotropic receptor 8(GRM8) |
| GRM8 | U92459 | Homo sapiens | glutamate metabotropic receptor 8(GRM8) |
| GRM8 | NM_000845 | Homo sapiens | glutamate metabotropic receptor 8(GRM8) |
| GRM8 | NM_001127323 | Homo sapiens | glutamate metabotropic receptor 8(GRM8) |
| GRM8 | XM_011516095 | Homo sapiens | glutamate metabotropic receptor 8(GRM8) |
| GRM8 | BG202742 | Homo sapiens | glutamate metabotropic receptor 8(GRM8) |
| GRM8 | XM_017012080 | Homo sapiens | glutamate metabotropic receptor 8(GRM8) |
| GRM8 | XM_011516108 | Homo sapiens | glutamate metabotropic receptor 8(GRM8) |
| GRM8 | EU432125 | Homo sapiens | glutamate metabotropic receptor 8(GRM8) |
| GRM8 | XM_011516103 | Homo sapiens | glutamate metabotropic receptor 8(GRM8) |
| GRM8 | AK290197 | Homo sapiens | glutamate metabotropic receptor 8(GRM8) |
| GRM8 | NR_028041 | Homo sapiens | glutamate metabotropic receptor 8(GRM8) |
| CFAP47 | BG197183 | Homo sapiens | cilia and flagella associated protein 47(CFAP47) |
| CFAP47 | CD686506 | Homo sapiens | cilia and flagella associated protein 47(CFAP47) |
| CFAP47 | DV080631 | Homo sapiens | cilia and flagella associated protein 47(CFAP47) |
| CFAP47 | XM_017029452 | Homo sapiens | cilia and flagella associated protein 47(CFAP47) |
| CFAP47 | DV080632 | Homo sapiens | cilia and flagella associated protein 47(CFAP47) |
| CFAP47 | XM_017029453 | Homo sapiens | cilia and flagella associated protein 47(CFAP47) |
| CFAP47 | AI648401 | Homo sapiens | cilia and flagella associated protein 47(CFAP47) |
| CFAP47 | BC101698 | Homo sapiens | cilia and flagella associated protein 47(CFAP47) |
| CFAP47 | DY655297 | Homo sapiens | cilia and flagella associated protein 47(CFAP47) |
| CFAP47 | BG190566 | Homo sapiens | cilia and flagella associated protein 47(CFAP47) |
| CFAP47 | BG200277 | Homo sapiens | cilia and flagella associated protein 47(CFAP47) |
| CFAP47 | CD106515 | Homo sapiens | cilia and flagella associated protein 47(CFAP47) |
| CFAP47 | AK093920 | Homo sapiens | cilia and flagella associated protein 47(CFAP47) |
| CFAP47 | DA315191 | Homo sapiens | cilia and flagella associated protein 47(CFAP47) |
| CFAP47 | BC027936 | Homo sapiens | cilia and flagella associated protein 47(CFAP47) |
| CFAP47 | DV080105 | Homo sapiens | cilia and flagella associated protein 47(CFAP47) |
| CFAP47 | DV080305 | Homo sapiens | cilia and flagella associated protein 47(CFAP47) |
| CFAP47 | NM_001304548 | Homo sapiens | cilia and flagella associated protein 47(CFAP47) |
| CFAP47 | DV080684 | Homo sapiens | cilia and flagella associated protein 47(CFAP47) |
| CFAP47 | NM_152632 | Homo sapiens | cilia and flagella associated protein 47(CFAP47) |
| CFAP47 | DV080141 | Homo sapiens | cilia and flagella associated protein 47(CFAP47) |
| CFAP47 | DV080142 | Homo sapiens | cilia and flagella associated protein 47(CFAP47) |
| CFAP47 | NM_173695 | Homo sapiens | cilia and flagella associated protein 47(CFAP47) |
| CFAP47 | AI822140 | Homo sapiens | cilia and flagella associated protein 47(CFAP47) |
| CFAP47 | DV080683 | Homo sapiens | cilia and flagella associated protein 47(CFAP47) |
| CFAP47 | BX111076 | Homo sapiens | cilia and flagella associated protein 47(CFAP47) |
| CFAP47 | BC101700 | Homo sapiens | cilia and flagella associated protein 47(CFAP47) |
| CFAP47 | AK126295 | Homo sapiens | cilia and flagella associated protein 47(CFAP47) |
| CFAP47 | DV080519 | Homo sapiens | cilia and flagella associated protein 47(CFAP47) |
| CFAP47 | DV080613 | Homo sapiens | cilia and flagella associated protein 47(CFAP47) |
| CFAP47 | BG186336 | Homo sapiens | cilia and flagella associated protein 47(CFAP47) |
| HYDIN | AK057467 | Homo sapiens | HYDIN, axonemal central pair apparatus protein(HYDIN) |
| HYDIN | DA758908 | Homo sapiens | HYDIN, axonemal central pair apparatus protein(HYDIN) |
| HYDIN | AK299016 | Homo sapiens | HYDIN, axonemal central pair apparatus protein(HYDIN) |
| HYDIN | XM_006721206 | Homo sapiens | HYDIN, axonemal central pair apparatus protein(HYDIN) |
| HYDIN | XM_011523155 | Homo sapiens | HYDIN, axonemal central pair apparatus protein(HYDIN) |
| HYDIN | AL137259 | Homo sapiens | HYDIN, axonemal central pair apparatus protein(HYDIN) |
| HYDIN | BC028351 | Homo sapiens | HYDIN, axonemal central pair apparatus protein(HYDIN) |
| HYDIN | AK026688 | Homo sapiens | HYDIN, axonemal central pair apparatus protein(HYDIN) |
| HYDIN | JX501991 | Homo sapiens | HYDIN, axonemal central pair apparatus protein(HYDIN) |
| HYDIN | DB339472 | Homo sapiens | HYDIN, axonemal central pair apparatus protein(HYDIN) |
| HYDIN | AL122038 | Homo sapiens | HYDIN, axonemal central pair apparatus protein(HYDIN) |
| HYDIN | DB079205 | Homo sapiens | HYDIN, axonemal central pair apparatus protein(HYDIN) |
| HYDIN | NM_032821 | Homo sapiens | HYDIN, axonemal central pair apparatus protein(HYDIN) |
| HYDIN | AL705531 | Homo sapiens | HYDIN, axonemal central pair apparatus protein(HYDIN) |
| HYDIN | AK299348 | Homo sapiens | HYDIN, axonemal central pair apparatus protein(HYDIN) |
| HYDIN | XM_011523147 | Homo sapiens | HYDIN, axonemal central pair apparatus protein(HYDIN) |
| HYDIN | XM_011523148 | Homo sapiens | HYDIN, axonemal central pair apparatus protein(HYDIN) |
| HYDIN | AL133042 | Homo sapiens | HYDIN, axonemal central pair apparatus protein(HYDIN) |
| HYDIN | NM_001270974 | Homo sapiens | HYDIN, axonemal central pair apparatus protein(HYDIN) |
| HYDIN | XM_011523146 | Homo sapiens | HYDIN, axonemal central pair apparatus protein(HYDIN) |
| HYDIN | BP228881 | Homo sapiens | HYDIN, axonemal central pair apparatus protein(HYDIN) |
| HYDIN | AK074472 | Homo sapiens | HYDIN, axonemal central pair apparatus protein(HYDIN) |
| HYDIN | BC043273 | Homo sapiens | HYDIN, axonemal central pair apparatus protein(HYDIN) |
| HYDIN | HG508232 | Homo sapiens | HYDIN, axonemal central pair apparatus protein(HYDIN) |
| HYDIN | NM_001198542 | Homo sapiens | HYDIN, axonemal central pair apparatus protein(HYDIN) |
| HYDIN | XM_017023347 | Homo sapiens | HYDIN, axonemal central pair apparatus protein(HYDIN) |
| HYDIN | NM_001198543 | Homo sapiens | HYDIN, axonemal central pair apparatus protein(HYDIN) |
| HYDIN | XM_017023346 | Homo sapiens | HYDIN, axonemal central pair apparatus protein(HYDIN) |
| HYDIN | AK022933 | Homo sapiens | HYDIN, axonemal central pair apparatus protein(HYDIN) |
| HYDIN | XM_017023348 | Homo sapiens | HYDIN, axonemal central pair apparatus protein(HYDIN) |
| HYDIN | XM_011523151 | Homo sapiens | HYDIN, axonemal central pair apparatus protein(HYDIN) |
| HYDIN | AK125886 | Homo sapiens | HYDIN, axonemal central pair apparatus protein(HYDIN) |
| HYDIN | XM_011523152 | Homo sapiens | HYDIN, axonemal central pair apparatus protein(HYDIN) |
| HYDIN | AK308296 | Homo sapiens | HYDIN, axonemal central pair apparatus protein(HYDIN) |
| HYDIN | NM_017558 | Homo sapiens | HYDIN, axonemal central pair apparatus protein(HYDIN) |
| RSPO3 | XM_017011378 | Homo sapiens | R-spondin 3(RSPO3) |
| RSPO3 | XM_017011379 | Homo sapiens | R-spondin 3(RSPO3) |
| RSPO3 | NM_032784 | Homo sapiens | R-spondin 3(RSPO3) |
| RSPO3 | DQ894583 | Homo sapiens | R-spondin 3(RSPO3) |
| RSPO3 | AA568446 | Homo sapiens | R-spondin 3(RSPO3) |
| RSPO3 | AI095468 | Homo sapiens | R-spondin 3(RSPO3) |
| RSPO3 | BC022367 | Homo sapiens | R-spondin 3(RSPO3) |
| RSPO3 | DQ891407 | Homo sapiens | R-spondin 3(RSPO3) |
| RSPO3 | AF251057 | Homo sapiens | R-spondin 3(RSPO3) |
| RSPO3 | AF086298 | Homo sapiens | R-spondin 3(RSPO3) |
| RSPO3 | AK314912 | Homo sapiens | R-spondin 3(RSPO3) |
| KDM6A | AF000992 | Homo sapiens | lysine demethylase 6A(KDM6A) |
| KDM6A | BC093868 | Homo sapiens | lysine demethylase 6A(KDM6A) |
| KDM6A | AK304114 | Homo sapiens | lysine demethylase 6A(KDM6A) |
| KDM6A | AF000993 | Homo sapiens | lysine demethylase 6A(KDM6A) |
| KDM6A | EU026283 | Homo sapiens | lysine demethylase 6A(KDM6A) |
| KDM6A | NM_001291421 | Homo sapiens | lysine demethylase 6A(KDM6A) |
| KDM6A | NR_111960 | Homo sapiens | lysine demethylase 6A(KDM6A) |
| KDM6A | AL041377 | Homo sapiens | lysine demethylase 6A(KDM6A) |
| KDM6A | CR749602 | Homo sapiens | lysine demethylase 6A(KDM6A) |
| KDM6A | NM_001291418 | Homo sapiens | lysine demethylase 6A(KDM6A) |
| KDM6A | NM_001291416 | Homo sapiens | lysine demethylase 6A(KDM6A) |
| KDM6A | XM_005272656 | Homo sapiens | lysine demethylase 6A(KDM6A) |
| KDM6A | NM_001291417 | Homo sapiens | lysine demethylase 6A(KDM6A) |
| KDM6A | XM_005272659 | Homo sapiens | lysine demethylase 6A(KDM6A) |
| KDM6A | XR_001755723 | Homo sapiens | lysine demethylase 6A(KDM6A) |
| KDM6A | XM_011543957 | Homo sapiens | lysine demethylase 6A(KDM6A) |
| KDM6A | XM_011543959 | Homo sapiens | lysine demethylase 6A(KDM6A) |
| KDM6A | XM_011543958 | Homo sapiens | lysine demethylase 6A(KDM6A) |
| KDM6A | AK294456 | Homo sapiens | lysine demethylase 6A(KDM6A) |
| KDM6A | BC143275 | Homo sapiens | lysine demethylase 6A(KDM6A) |
| KDM6A | BC143277 | Homo sapiens | lysine demethylase 6A(KDM6A) |
| KDM6A | XM_011543960 | Homo sapiens | lysine demethylase 6A(KDM6A) |
| KDM6A | BC143278 | Homo sapiens | lysine demethylase 6A(KDM6A) |
| KDM6A | XM_011543962 | Homo sapiens | lysine demethylase 6A(KDM6A) |
| KDM6A | XM_011543961 | Homo sapiens | lysine demethylase 6A(KDM6A) |
| KDM6A | XM_011543964 | Homo sapiens | lysine demethylase 6A(KDM6A) |
| KDM6A | XM_011543963 | Homo sapiens | lysine demethylase 6A(KDM6A) |
| KDM6A | XM_011543966 | Homo sapiens | lysine demethylase 6A(KDM6A) |
| KDM6A | XM_011543965 | Homo sapiens | lysine demethylase 6A(KDM6A) |
| KDM6A | BC113381 | Homo sapiens | lysine demethylase 6A(KDM6A) |
| KDM6A | XM_011543968 | Homo sapiens | lysine demethylase 6A(KDM6A) |
| KDM6A | XM_011543967 | Homo sapiens | lysine demethylase 6A(KDM6A) |
| KDM6A | XM_011543969 | Homo sapiens | lysine demethylase 6A(KDM6A) |
| KDM6A | AL831996 | Homo sapiens | lysine demethylase 6A(KDM6A) |
| KDM6A | DQ062677 | Homo sapiens | lysine demethylase 6A(KDM6A) |
| KDM6A | DQ062676 | Homo sapiens | lysine demethylase 6A(KDM6A) |
| KDM6A | DQ062678 | Homo sapiens | lysine demethylase 6A(KDM6A) |
| KDM6A | NM_021140 | Homo sapiens | lysine demethylase 6A(KDM6A) |
| KDM6A | AK307691 | Homo sapiens | lysine demethylase 6A(KDM6A) |
| KDM6A | AB208795 | Homo sapiens | lysine demethylase 6A(KDM6A) |
| KDM6A | XM_017029782 | Homo sapiens | lysine demethylase 6A(KDM6A) |
| KDM6A | EU026291 | Homo sapiens | lysine demethylase 6A(KDM6A) |
| KDM6A | XM_017029783 | Homo sapiens | lysine demethylase 6A(KDM6A) |
| KDM6A | XM_011543971 | Homo sapiens | lysine demethylase 6A(KDM6A) |
| KDM6A | XM_011543970 | Homo sapiens | lysine demethylase 6A(KDM6A) |
| KDM6A | AI364446 | Homo sapiens | lysine demethylase 6A(KDM6A) |
| KDM6A | AK303430 | Homo sapiens | lysine demethylase 6A(KDM6A) |
| KDM6A | XM_011543973 | Homo sapiens | lysine demethylase 6A(KDM6A) |
| KDM6A | XM_011543972 | Homo sapiens | lysine demethylase 6A(KDM6A) |
| KDM6A | DC381812 | Homo sapiens | lysine demethylase 6A(KDM6A) |
| KDM6A | XM_011543975 | Homo sapiens | lysine demethylase 6A(KDM6A) |
| KDM6A | NM_001291415 | Homo sapiens | lysine demethylase 6A(KDM6A) |
| KDM6A | XM_011543974 | Homo sapiens | lysine demethylase 6A(KDM6A) |
| KDM6A | XM_017029784 | Homo sapiens | lysine demethylase 6A(KDM6A) |
| KDM6A | XM_017029785 | Homo sapiens | lysine demethylase 6A(KDM6A) |
| KDM6A | BX640694 | Homo sapiens | lysine demethylase 6A(KDM6A) |
| KDM6A | BC143272 | Homo sapiens | lysine demethylase 6A(KDM6A) |
| MYOG | BC053899 | Homo sapiens | myogenin(MYOG) |
| MYOG | BT007233 | Homo sapiens | myogenin(MYOG) |
| MYOG | X17651 | Homo sapiens | myogenin(MYOG) |
| MYOG | AW469592 | Homo sapiens | myogenin(MYOG) |
| MYOG | BF204429 | Homo sapiens | myogenin(MYOG) |
| MYOG | EU831793 | Homo sapiens | myogenin(MYOG) |
| MYOG | EU831870 | Homo sapiens | myogenin(MYOG) |
| MYOG | NM_002479 | Homo sapiens | myogenin(MYOG) |
| GRID2 | AK308611 | Homo sapiens | glutamate ionotropic receptor delta type subunit 2(GRID2) |
| GRID2 | XM_017008124 | Homo sapiens | glutamate ionotropic receptor delta type subunit 2(GRID2) |
| GRID2 | AW295056 | Homo sapiens | glutamate ionotropic receptor delta type subunit 2(GRID2) |
| GRID2 | XM_017008123 | Homo sapiens | glutamate ionotropic receptor delta type subunit 2(GRID2) |
| GRID2 | XM_017008122 | Homo sapiens | glutamate ionotropic receptor delta type subunit 2(GRID2) |
| GRID2 | XM_017008121 | Homo sapiens | glutamate ionotropic receptor delta type subunit 2(GRID2) |
| GRID2 | XM_017008120 | Homo sapiens | glutamate ionotropic receptor delta type subunit 2(GRID2) |
| GRID2 | AK310262 | Homo sapiens | glutamate ionotropic receptor delta type subunit 2(GRID2) |
| GRID2 | AK302355 | Homo sapiens | glutamate ionotropic receptor delta type subunit 2(GRID2) |
| GRID2 | XM_017008119 | Homo sapiens | glutamate ionotropic receptor delta type subunit 2(GRID2) |
| GRID2 | NM_001510 | Homo sapiens | glutamate ionotropic receptor delta type subunit 2(GRID2) |
| GRID2 | XM_017008118 | Homo sapiens | glutamate ionotropic receptor delta type subunit 2(GRID2) |
| GRID2 | XM_017008127 | Homo sapiens | glutamate ionotropic receptor delta type subunit 2(GRID2) |
| GRID2 | XM_017008126 | Homo sapiens | glutamate ionotropic receptor delta type subunit 2(GRID2) |
| GRID2 | XM_017008125 | Homo sapiens | glutamate ionotropic receptor delta type subunit 2(GRID2) |
| GRID2 | BC099653 | Homo sapiens | glutamate ionotropic receptor delta type subunit 2(GRID2) |
| GRID2 | BC099652 | Homo sapiens | glutamate ionotropic receptor delta type subunit 2(GRID2) |
| GRID2 | BC099654 | Homo sapiens | glutamate ionotropic receptor delta type subunit 2(GRID2) |
| GRID2 | NM_001286838 | Homo sapiens | glutamate ionotropic receptor delta type subunit 2(GRID2) |
| GRID2 | XM_011531893 | Homo sapiens | glutamate ionotropic receptor delta type subunit 2(GRID2) |
| GRID2 | XM_011531895 | Homo sapiens | glutamate ionotropic receptor delta type subunit 2(GRID2) |
| GRID2 | AF009014 | Homo sapiens | glutamate ionotropic receptor delta type subunit 2(GRID2) |
| GRID2 | XM_011531894 | Homo sapiens | glutamate ionotropic receptor delta type subunit 2(GRID2) |
| GRID2 | AB209318 | Homo sapiens | glutamate ionotropic receptor delta type subunit 2(GRID2) |
| ADGRV1 | XM_017009963 | Homo sapiens | adhesion G protein-coupled receptor V1(ADGRV1) |
| ADGRV1 | AL136541 | Homo sapiens | adhesion G protein-coupled receptor V1(ADGRV1) |
| ADGRV1 | XM_017009964 | Homo sapiens | adhesion G protein-coupled receptor V1(ADGRV1) |
| ADGRV1 | XM_017009965 | Homo sapiens | adhesion G protein-coupled receptor V1(ADGRV1) |
| ADGRV1 | XM_017009966 | Homo sapiens | adhesion G protein-coupled receptor V1(ADGRV1) |
| ADGRV1 | XM_017009967 | Homo sapiens | adhesion G protein-coupled receptor V1(ADGRV1) |
| ADGRV1 | XM_017009968 | Homo sapiens | adhesion G protein-coupled receptor V1(ADGRV1) |
| ADGRV1 | XM_017009969 | Homo sapiens | adhesion G protein-coupled receptor V1(ADGRV1) |
| ADGRV1 | AF055084 | Homo sapiens | adhesion G protein-coupled receptor V1(ADGRV1) |
| ADGRV1 | AF435925 | Homo sapiens | adhesion G protein-coupled receptor V1(ADGRV1) |
| ADGRV1 | BC034748 | Homo sapiens | adhesion G protein-coupled receptor V1(ADGRV1) |
| ADGRV1 | BX493741 | Homo sapiens | adhesion G protein-coupled receptor V1(ADGRV1) |
| ADGRV1 | AB014586 | Homo sapiens | adhesion G protein-coupled receptor V1(ADGRV1) |
| ADGRV1 | AB075823 | Homo sapiens | adhesion G protein-coupled receptor V1(ADGRV1) |
| ADGRV1 | XM_017009973 | Homo sapiens | adhesion G protein-coupled receptor V1(ADGRV1) |
| ADGRV1 | XM_017009974 | Homo sapiens | adhesion G protein-coupled receptor V1(ADGRV1) |
| ADGRV1 | DA503892 | Homo sapiens | adhesion G protein-coupled receptor V1(ADGRV1) |
| ADGRV1 | AL133041 | Homo sapiens | adhesion G protein-coupled receptor V1(ADGRV1) |
| ADGRV1 | XM_017009970 | Homo sapiens | adhesion G protein-coupled receptor V1(ADGRV1) |
| ADGRV1 | XM_017009971 | Homo sapiens | adhesion G protein-coupled receptor V1(ADGRV1) |
| ADGRV1 | XM_017009972 | Homo sapiens | adhesion G protein-coupled receptor V1(ADGRV1) |
| ADGRV1 | AK024416 | Homo sapiens | adhesion G protein-coupled receptor V1(ADGRV1) |
| ADGRV1 | BI758469 | Homo sapiens | adhesion G protein-coupled receptor V1(ADGRV1) |
| ADGRV1 | NR_003149 | Homo sapiens | adhesion G protein-coupled receptor V1(ADGRV1) |
| ADGRV1 | CD633594 | Homo sapiens | adhesion G protein-coupled receptor V1(ADGRV1) |
| ADGRV1 | NM_032119 | Homo sapiens | adhesion G protein-coupled receptor V1(ADGRV1) |
| ITGA4 | AJ504733 | Homo sapiens | integrin subunit alpha 4(ITGA4) |
| ITGA4 | X16983 | Homo sapiens | integrin subunit alpha 4(ITGA4) |
| ITGA4 | BC080190 | Homo sapiens | integrin subunit alpha 4(ITGA4) |
| ITGA4 | BC146277 | Homo sapiens | integrin subunit alpha 4(ITGA4) |
| ITGA4 | NM_000885 | Homo sapiens | integrin subunit alpha 4(ITGA4) |
| ITGA4 | AK311738 | Homo sapiens | integrin subunit alpha 4(ITGA4) |
| ITGA4 | AB208885 | Homo sapiens | integrin subunit alpha 4(ITGA4) |
| ITGA4 | AK303220 | Homo sapiens | integrin subunit alpha 4(ITGA4) |
| ITGA4 | AJ508356 | Homo sapiens | integrin subunit alpha 4(ITGA4) |
| ITGA4 | AK303478 | Homo sapiens | integrin subunit alpha 4(ITGA4) |
| ITGA4 | BU689519 | Homo sapiens | integrin subunit alpha 4(ITGA4) |
| ITGA4 | NM_001316312 | Homo sapiens | integrin subunit alpha 4(ITGA4) |
| ITGA4 | BC055419 | Homo sapiens | integrin subunit alpha 4(ITGA4) |
| ITGA4 | DB114726 | Homo sapiens | integrin subunit alpha 4(ITGA4) |
| ITGA4 | BC156712 | Homo sapiens | integrin subunit alpha 4(ITGA4) |
| ITGA4 | AJ510248 | Homo sapiens | integrin subunit alpha 4(ITGA4) |
| ITGA4 | AJ510247 | Homo sapiens | integrin subunit alpha 4(ITGA4) |
| ITGA4 | AJ510246 | Homo sapiens | integrin subunit alpha 4(ITGA4) |
| ITGA4 | AK310610 | Homo sapiens | integrin subunit alpha 4(ITGA4) |
| ITGA4 | AJ510249 | Homo sapiens | integrin subunit alpha 4(ITGA4) |
| ITGA4 | BC016671 | Homo sapiens | integrin subunit alpha 4(ITGA4) |
| ITGA4 | L12002 | Homo sapiens | integrin subunit alpha 4(ITGA4) |
| PAX3 | U02309 | Homo sapiens | paired box 3(PAX3) |
| PAX3 | S69370 | Homo sapiens | paired box 3(PAX3) |
| PAX3 | BC101299 | Homo sapiens | paired box 3(PAX3) |
| PAX3 | NM_181461 | Homo sapiens | paired box 3(PAX3) |
| PAX3 | NM_181460 | Homo sapiens | paired box 3(PAX3) |
| PAX3 | AI399816 | Homo sapiens | paired box 3(PAX3) |
| PAX3 | NM_000438 | Homo sapiens | paired box 3(PAX3) |
| PAX3 | L07483 | Homo sapiens | paired box 3(PAX3) |
| PAX3 | NM_013942 | Homo sapiens | paired box 3(PAX3) |
| PAX3 | S69369 | Homo sapiens | paired box 3(PAX3) |
| PAX3 | CA389778 | Homo sapiens | paired box 3(PAX3) |
| PAX3 | AK291278 | Homo sapiens | paired box 3(PAX3) |
| PAX3 | AY251280 | Homo sapiens | paired box 3(PAX3) |
| PAX3 | NM_181458 | Homo sapiens | paired box 3(PAX3) |
| PAX3 | NM_181457 | Homo sapiens | paired box 3(PAX3) |
| PAX3 | BC101302 | Homo sapiens | paired box 3(PAX3) |
| PAX3 | NM_181459 | Homo sapiens | paired box 3(PAX3) |
| PAX3 | BC063547 | Homo sapiens | paired box 3(PAX3) |
| PAX3 | BC101301 | Homo sapiens | paired box 3(PAX3) |
| PAX3 | BC101300 | Homo sapiens | paired box 3(PAX3) |
| PAX3 | EU446645 | Homo sapiens | paired box 3(PAX3) |
| PAX3 | BC114363 | Homo sapiens | paired box 3(PAX3) |
| PAX3 | DN989642 | Homo sapiens | paired box 3(PAX3) |
| PAX3 | NM_001127366 | Homo sapiens | paired box 3(PAX3) |
| PAX3 | AI382779 | Homo sapiens | paired box 3(PAX3) |
| PAX3 | AY251279 | Homo sapiens | paired box 3(PAX3) |
| PAX3 | DA758804 | Homo sapiens | paired box 3(PAX3) |
| NRG1 | XM_017013369 | Homo sapiens | neuregulin 1(NRG1) |
| NRG1 | U02329 | Homo sapiens | neuregulin 1(NRG1) |
| NRG1 | U02328 | Homo sapiens | neuregulin 1(NRG1) |
| NRG1 | U02327 | Homo sapiens | neuregulin 1(NRG1) |
| NRG1 | XM_011544512 | Homo sapiens | neuregulin 1(NRG1) |
| NRG1 | AF026146 | Homo sapiens | neuregulin 1(NRG1) |
| NRG1 | U02326 | Homo sapiens | neuregulin 1(NRG1) |
| NRG1 | BC007675 | Homo sapiens | neuregulin 1(NRG1) |
| NRG1 | BC064587 | Homo sapiens | neuregulin 1(NRG1) |
| NRG1 | BT007389 | Homo sapiens | neuregulin 1(NRG1) |
| NRG1 | DC425692 | Homo sapiens | neuregulin 1(NRG1) |
| NRG1 | NM_013964 | Homo sapiens | neuregulin 1(NRG1) |
| NRG1 | AF176921 | Homo sapiens | neuregulin 1(NRG1) |
| NRG1 | AK097005 | Homo sapiens | neuregulin 1(NRG1) |
| NRG1 | NM_013962 | Homo sapiens | neuregulin 1(NRG1) |
| NRG1 | NM_013960 | Homo sapiens | neuregulin 1(NRG1) |
| NRG1 | DA805080 | Homo sapiens | neuregulin 1(NRG1) |
| NRG1 | L12261 | Homo sapiens | neuregulin 1(NRG1) |
| NRG1 | XM_017013368 | Homo sapiens | neuregulin 1(NRG1) |
| NRG1 | XM_017013367 | Homo sapiens | neuregulin 1(NRG1) |
| NRG1 | U02330 | Homo sapiens | neuregulin 1(NRG1) |
| NRG1 | XM_017013366 | Homo sapiens | neuregulin 1(NRG1) |
| NRG1 | XM_017013365 | Homo sapiens | neuregulin 1(NRG1) |
| NRG1 | AK293240 | Homo sapiens | neuregulin 1(NRG1) |
| NRG1 | L12260 | Homo sapiens | neuregulin 1(NRG1) |
| NRG1 | AK298132 | Homo sapiens | neuregulin 1(NRG1) |
| NRG1 | EU359055 | Homo sapiens | neuregulin 1(NRG1) |
| NRG1 | AB451331 | Homo sapiens | neuregulin 1(NRG1) |
| NRG1 | AK223435 | Homo sapiens | neuregulin 1(NRG1) |
| NRG1 | EF372274 | Homo sapiens | neuregulin 1(NRG1) |
| NRG1 | M94165 | Homo sapiens | neuregulin 1(NRG1) |
| NRG1 | EF372275 | Homo sapiens | neuregulin 1(NRG1) |
| NRG1 | M94166 | Homo sapiens | neuregulin 1(NRG1) |
| NRG1 | M94167 | Homo sapiens | neuregulin 1(NRG1) |
| NRG1 | EF372273 | Homo sapiens | neuregulin 1(NRG1) |
| NRG1 | M94168 | Homo sapiens | neuregulin 1(NRG1) |
| NRG1 | BC006492 | Homo sapiens | neuregulin 1(NRG1) |
| NRG1 | EF372276 | Homo sapiens | neuregulin 1(NRG1) |
| NRG1 | EU363510 | Homo sapiens | neuregulin 1(NRG1) |
| NRG1 | EF372277 | Homo sapiens | neuregulin 1(NRG1) |
| NRG1 | GQ983557 | Homo sapiens | neuregulin 1(NRG1) |
| NRG1 | L41827 | Homo sapiens | neuregulin 1(NRG1) |
| NRG1 | GQ983558 | Homo sapiens | neuregulin 1(NRG1) |
| NRG1 | CN603661 | Homo sapiens | neuregulin 1(NRG1) |
| NRG1 | NM_004495 | Homo sapiens | neuregulin 1(NRG1) |
| NRG1 | NM_001159999 | Homo sapiens | neuregulin 1(NRG1) |
| NRG1 | NM_001322201 | Homo sapiens | neuregulin 1(NRG1) |
| NRG1 | NM_001322202 | Homo sapiens | neuregulin 1(NRG1) |
| NRG1 | NM_001322203 | Homo sapiens | neuregulin 1(NRG1) |
| NRG1 | NM_013959 | Homo sapiens | neuregulin 1(NRG1) |
| NRG1 | NM_001159995 | Homo sapiens | neuregulin 1(NRG1) |
| NRG1 | NM_001322205 | Homo sapiens | neuregulin 1(NRG1) |
| NRG1 | NM_013958 | Homo sapiens | neuregulin 1(NRG1) |
| NRG1 | AK290730 | Homo sapiens | neuregulin 1(NRG1) |
| NRG1 | NM_001159996 | Homo sapiens | neuregulin 1(NRG1) |
| NRG1 | NM_001322206 | Homo sapiens | neuregulin 1(NRG1) |
| NRG1 | NM_013957 | Homo sapiens | neuregulin 1(NRG1) |
| NRG1 | NM_001322207 | Homo sapiens | neuregulin 1(NRG1) |
| NRG1 | NM_013956 | Homo sapiens | neuregulin 1(NRG1) |
| NRG1 | CA429204 | Homo sapiens | neuregulin 1(NRG1) |
| NRG1 | AK293270 | Homo sapiens | neuregulin 1(NRG1) |
| NRG1 | EU363509 | Homo sapiens | neuregulin 1(NRG1) |
| NRG1 | NM_001322197 | Homo sapiens | neuregulin 1(NRG1) |
| NRG1 | BC144446 | Homo sapiens | neuregulin 1(NRG1) |
| NRG1 | EU363508 | Homo sapiens | neuregulin 1(NRG1) |
| NRG1 | XM_005273486 | Homo sapiens | neuregulin 1(NRG1) |
| NRG1 | BC073871 | Homo sapiens | neuregulin 1(NRG1) |
| NRG1 | CN603654 | Homo sapiens | neuregulin 1(NRG1) |
| NRG1 | CN603652 | Homo sapiens | neuregulin 1(NRG1) |
| NRG1 | EF517297 | Homo sapiens | neuregulin 1(NRG1) |
| NRG1 | XM_005273487 | Homo sapiens | neuregulin 1(NRG1) |
| NRG1 | AY207002 | Homo sapiens | neuregulin 1(NRG1) |
| NRG1 | EF517295 | Homo sapiens | neuregulin 1(NRG1) |
| NRG1 | EF517296 | Homo sapiens | neuregulin 1(NRG1) |
| NRG1 | CR450288 | Homo sapiens | neuregulin 1(NRG1) |
| NRG1 | BC150609 | Homo sapiens | neuregulin 1(NRG1) |
| NRG1 | HG501045 | Homo sapiens | neuregulin 1(NRG1) |
| NRG1 | NM_001160001 | Homo sapiens | neuregulin 1(NRG1) |
| NRG1 | AB451475 | Homo sapiens | neuregulin 1(NRG1) |
| NRG1 | NM_001160008 | Homo sapiens | neuregulin 1(NRG1) |
| NRG1 | NM_001160007 | Homo sapiens | neuregulin 1(NRG1) |
| NRG1 | S68256 | Homo sapiens | neuregulin 1(NRG1) |
| NRG1 | NM_001160004 | Homo sapiens | neuregulin 1(NRG1) |
| NRG1 | NM_001160005 | Homo sapiens | neuregulin 1(NRG1) |
| NRG1 | NM_001160002 | Homo sapiens | neuregulin 1(NRG1) |
| NRG1 | AK289927 | Homo sapiens | neuregulin 1(NRG1) |
| NRG1 | BQ018711 | Homo sapiens | neuregulin 1(NRG1) |
| NRG1 | XM_017013371 | Homo sapiens | neuregulin 1(NRG1) |
| NRG1 | XM_017013370 | Homo sapiens | neuregulin 1(NRG1) |
| NRG1 | U02325 | Homo sapiens | neuregulin 1(NRG1) |
| NRG1 | XM_006716335 | Homo sapiens | neuregulin 1(NRG1) |
| NRG1 | XM_017013372 | Homo sapiens | neuregulin 1(NRG1) |
| PAX6 | BX440968 | Homo sapiens | paired box 6(PAX6) |
| PAX6 | DQ894612 | Homo sapiens | paired box 6(PAX6) |
| PAX6 | AY047583 | Homo sapiens | paired box 6(PAX6) |
| PAX6 | CA397106 | Homo sapiens | paired box 6(PAX6) |
| PAX6 | CV569250 | Homo sapiens | paired box 6(PAX6) |
| PAX6 | BM557761 | Homo sapiens | paired box 6(PAX6) |
| PAX6 | KP255960 | Homo sapiens | paired box 6(PAX6) |
| PAX6 | AB593092 | Homo sapiens | paired box 6(PAX6) |
| PAX6 | AK094172 | Homo sapiens | paired box 6(PAX6) |
| PAX6 | AK074881 | Homo sapiens | paired box 6(PAX6) |
| PAX6 | BM725029 | Homo sapiens | paired box 6(PAX6) |
| PAX6 | AB209177 | Homo sapiens | paired box 6(PAX6) |
| PAX6 | AB593094 | Homo sapiens | paired box 6(PAX6) |
| PAX6 | AB593093 | Homo sapiens | paired box 6(PAX6) |
| PAX6 | AK314470 | Homo sapiens | paired box 6(PAX6) |
| PAX6 | DA183294 | Homo sapiens | paired box 6(PAX6) |
| PAX6 | NM_001127612 | Homo sapiens | paired box 6(PAX6) |
| PAX6 | DA141443 | Homo sapiens | paired box 6(PAX6) |
| PAX6 | BP394576 | Homo sapiens | paired box 6(PAX6) |
| PAX6 | DA089215 | Homo sapiens | paired box 6(PAX6) |
| PAX6 | CA397536 | Homo sapiens | paired box 6(PAX6) |
| PAX6 | NM_001310158 | Homo sapiens | paired box 6(PAX6) |
| PAX6 | BX114225 | Homo sapiens | paired box 6(PAX6) |
| PAX6 | M77844 | Homo sapiens | paired box 6(PAX6) |
| PAX6 | NM_001310159 | Homo sapiens | paired box 6(PAX6) |
| PAX6 | DA571138 | Homo sapiens | paired box 6(PAX6) |
| PAX6 | BM313099 | Homo sapiens | paired box 6(PAX6) |
| PAX6 | BE221553 | Homo sapiens | paired box 6(PAX6) |
| PAX6 | NM_001604 | Homo sapiens | paired box 6(PAX6) |
| PAX6 | BM666662 | Homo sapiens | paired box 6(PAX6) |
| PAX6 | BC011953 | Homo sapiens | paired box 6(PAX6) |
| PAX6 | BX089704 | Homo sapiens | paired box 6(PAX6) |
| PAX6 | NM_001258463 | Homo sapiens | paired box 6(PAX6) |
| PAX6 | NM_001258462 | Homo sapiens | paired box 6(PAX6) |
| PAX6 | NM_001258465 | Homo sapiens | paired box 6(PAX6) |
| PAX6 | NM_001258464 | Homo sapiens | paired box 6(PAX6) |
| PAX6 | BP394398 | Homo sapiens | paired box 6(PAX6) |
| PAX6 | DA079367 | Homo sapiens | paired box 6(PAX6) |
| PAX6 | NM_001310161 | Homo sapiens | paired box 6(PAX6) |
| PAX6 | GQ141695 | Homo sapiens | paired box 6(PAX6) |
| PAX6 | NM_001310160 | Homo sapiens | paired box 6(PAX6) |
| PAX6 | DA056636 | Homo sapiens | paired box 6(PAX6) |
| PAX6 | BU072567 | Homo sapiens | paired box 6(PAX6) |
| PAX6 | BX640762 | Homo sapiens | paired box 6(PAX6) |
| PAX6 | NM_000280 | Homo sapiens | paired box 6(PAX6) |
| PAX6 | BI789171 | Homo sapiens | paired box 6(PAX6) |
| PAX6 | AY707088 | Homo sapiens | paired box 6(PAX6) |
| PAX6 | F07809 | Homo sapiens | paired box 6(PAX6) |
| PAX6 | DA078958 | Homo sapiens | paired box 6(PAX6) |
| PAX6 | DQ891436 | Homo sapiens | paired box 6(PAX6) |
| PAX6 | BI816814 | Homo sapiens | paired box 6(PAX6) |
| PAX6 | M93650 | Homo sapiens | paired box 6(PAX6) |
| PAX6 | AK094249 | Homo sapiens | paired box 6(PAX6) |
| PAX6 | CD673930 | Homo sapiens | paired box 6(PAX6) |
| SYP | AK313030 | Homo sapiens | synaptophysin(SYP) |
| SYP | AK094376 | Homo sapiens | synaptophysin(SYP) |
| SYP | AK315953 | Homo sapiens | synaptophysin(SYP) |
| SYP | NM_003179 | Homo sapiens | synaptophysin(SYP) |
| SYP | BC064550 | Homo sapiens | synaptophysin(SYP) |
| SYP | BQ637168 | Homo sapiens | synaptophysin(SYP) |
| SYP | AK295524 | Homo sapiens | synaptophysin(SYP) |
| SYP | HQ448085 | Homo sapiens | synaptophysin(SYP) |
| SYP | X06389 | Homo sapiens | synaptophysin(SYP) |
| SYP | BC032385 | Homo sapiens | synaptophysin(SYP) |
| GFRA2 | XM_011544484 | Homo sapiens | GDNF family receptor alpha 2(GFRA2) |
| GFRA2 | BC041688 | Homo sapiens | GDNF family receptor alpha 2(GFRA2) |
| GFRA2 | AY326396 | Homo sapiens | GDNF family receptor alpha 2(GFRA2) |
| GFRA2 | U93703 | Homo sapiens | GDNF family receptor alpha 2(GFRA2) |
| GFRA2 | NM_001165038 | Homo sapiens | GDNF family receptor alpha 2(GFRA2) |
| GFRA2 | AF002700 | Homo sapiens | GDNF family receptor alpha 2(GFRA2) |
| GFRA2 | AY941828 | Homo sapiens | GDNF family receptor alpha 2(GFRA2) |
| GFRA2 | NM_001165039 | Homo sapiens | GDNF family receptor alpha 2(GFRA2) |
| GFRA2 | JF432396 | Homo sapiens | GDNF family receptor alpha 2(GFRA2) |
| GFRA2 | NM_001495 | Homo sapiens | GDNF family receptor alpha 2(GFRA2) |
| GFRA2 | U97145 | Homo sapiens | GDNF family receptor alpha 2(GFRA2) |
| GFRA2 | XM_006716327 | Homo sapiens | GDNF family receptor alpha 2(GFRA2) |
| GFRA2 | AK299623 | Homo sapiens | GDNF family receptor alpha 2(GFRA2) |
| SYN1 | NM_006950 | Homo sapiens | synapsin I(SYN1) |
| SYN1 | BC036711 | Homo sapiens | synapsin I(SYN1) |
| SYN1 | BC048799 | Homo sapiens | synapsin I(SYN1) |
| SYN1 | AL833961 | Homo sapiens | synapsin I(SYN1) |
| SYN1 | NM_133499 | Homo sapiens | synapsin I(SYN1) |
| SYN1 | AI929645 | Homo sapiens | synapsin I(SYN1) |
| PAX2 | BC148710 | Homo sapiens | paired box 2(PAX2) |
| PAX2 | BC141452 | Homo sapiens | paired box 2(PAX2) |
| PAX2 | NM_000278 | Homo sapiens | paired box 2(PAX2) |
| PAX2 | BJ989901 | Homo sapiens | paired box 2(PAX2) |
| PAX2 | BM671839 | Homo sapiens | paired box 2(PAX2) |
| PAX2 | M89470 | Homo sapiens | paired box 2(PAX2) |
| PAX2 | NM_003987 | Homo sapiens | paired box 2(PAX2) |
| PAX2 | L25597 | Homo sapiens | paired box 2(PAX2) |
| PAX2 | AY153484 | Homo sapiens | paired box 2(PAX2) |
| PAX2 | NM_003989 | Homo sapiens | paired box 2(PAX2) |
| PAX2 | AY153483 | Homo sapiens | paired box 2(PAX2) |
| PAX2 | NM_003988 | Homo sapiens | paired box 2(PAX2) |
| PAX2 | DB077633 | Homo sapiens | paired box 2(PAX2) |
| PAX2 | XM_017016284 | Homo sapiens | paired box 2(PAX2) |
| PAX2 | XM_017016283 | Homo sapiens | paired box 2(PAX2) |
| PAX2 | XM_017016286 | Homo sapiens | paired box 2(PAX2) |
| PAX2 | XM_017016285 | Homo sapiens | paired box 2(PAX2) |
| PAX2 | NM_003990 | Homo sapiens | paired box 2(PAX2) |
| PAX2 | XM_017016282 | Homo sapiens | paired box 2(PAX2) |
| PAX2 | XM_017016281 | Homo sapiens | paired box 2(PAX2) |
| PAX2 | XM_017016288 | Homo sapiens | paired box 2(PAX2) |
| PAX2 | XM_017016287 | Homo sapiens | paired box 2(PAX2) |
| PAX2 | NM_001304569 | Homo sapiens | paired box 2(PAX2) |
| PAX2 | XM_017016289 | Homo sapiens | paired box 2(PAX2) |
| PARD3B | AK056157 | Homo sapiens | par-3 family cell polarity regulator beta(PARD3B) |
| PARD3B | BC172499 | Homo sapiens | par-3 family cell polarity regulator beta(PARD3B) |
| PARD3B | XM_017003284 | Homo sapiens | par-3 family cell polarity regulator beta(PARD3B) |
| PARD3B | XM_017003283 | Homo sapiens | par-3 family cell polarity regulator beta(PARD3B) |
| PARD3B | BC012984 | Homo sapiens | par-3 family cell polarity regulator beta(PARD3B) |
| PARD3B | XM_017003289 | Homo sapiens | par-3 family cell polarity regulator beta(PARD3B) |
| PARD3B | XM_017003286 | Homo sapiens | par-3 family cell polarity regulator beta(PARD3B) |
| PARD3B | NM_205863 | Homo sapiens | par-3 family cell polarity regulator beta(PARD3B) |
| PARD3B | XM_017003285 | Homo sapiens | par-3 family cell polarity regulator beta(PARD3B) |
| PARD3B | AF428251 | Homo sapiens | par-3 family cell polarity regulator beta(PARD3B) |
| PARD3B | BC156252 | Homo sapiens | par-3 family cell polarity regulator beta(PARD3B) |
| PARD3B | NM_057177 | Homo sapiens | par-3 family cell polarity regulator beta(PARD3B) |
| PARD3B | XM_017003288 | Homo sapiens | par-3 family cell polarity regulator beta(PARD3B) |
| PARD3B | AF428250 | Homo sapiens | par-3 family cell polarity regulator beta(PARD3B) |
| PARD3B | NM_152526 | Homo sapiens | par-3 family cell polarity regulator beta(PARD3B) |
| PARD3B | XM_017003287 | Homo sapiens | par-3 family cell polarity regulator beta(PARD3B) |
| PARD3B | XM_011510553 | Homo sapiens | par-3 family cell polarity regulator beta(PARD3B) |
| PARD3B | AF466152 | Homo sapiens | par-3 family cell polarity regulator beta(PARD3B) |
| PARD3B | XM_011510552 | Homo sapiens | par-3 family cell polarity regulator beta(PARD3B) |
| PARD3B | NM_001302769 | Homo sapiens | par-3 family cell polarity regulator beta(PARD3B) |
| PARD3B | AK057965 | Homo sapiens | par-3 family cell polarity regulator beta(PARD3B) |
| PARD3B | AW274720 | Homo sapiens | par-3 family cell polarity regulator beta(PARD3B) |
| PARD3B | XM_017003293 | Homo sapiens | par-3 family cell polarity regulator beta(PARD3B) |
| PARD3B | BF434006 | Homo sapiens | par-3 family cell polarity regulator beta(PARD3B) |
| PARD3B | XM_017003292 | Homo sapiens | par-3 family cell polarity regulator beta(PARD3B) |
| PARD3B | XM_017003294 | Homo sapiens | par-3 family cell polarity regulator beta(PARD3B) |
| PARD3B | AI742400 | Homo sapiens | par-3 family cell polarity regulator beta(PARD3B) |
| PARD3B | XM_017003291 | Homo sapiens | par-3 family cell polarity regulator beta(PARD3B) |
| PARD3B | XM_017003290 | Homo sapiens | par-3 family cell polarity regulator beta(PARD3B) |
| PARD3B | AB073472 | Homo sapiens | par-3 family cell polarity regulator beta(PARD3B) |
| PARD3B | AK312083 | Homo sapiens | par-3 family cell polarity regulator beta(PARD3B) |
| PARD3B | AL832951 | Homo sapiens | par-3 family cell polarity regulator beta(PARD3B) |
| PARD3B | AB053321 | Homo sapiens | par-3 family cell polarity regulator beta(PARD3B) |
| PARD3B | AB092439 | Homo sapiens | par-3 family cell polarity regulator beta(PARD3B) |
| CCNA2 | BP363915 | Homo sapiens | cyclin A2(CCNA2) |
| CCNA2 | CR407692 | Homo sapiens | cyclin A2(CCNA2) |
| CCNA2 | AW276578 | Homo sapiens | cyclin A2(CCNA2) |
| CCNA2 | BC104783 | Homo sapiens | cyclin A2(CCNA2) |
| CCNA2 | AK291931 | Homo sapiens | cyclin A2(CCNA2) |
| CCNA2 | BC104787 | Homo sapiens | cyclin A2(CCNA2) |
| CCNA2 | NM_001237 | Homo sapiens | cyclin A2(CCNA2) |
| CCNA2 | X51688 | Homo sapiens | cyclin A2(CCNA2) |
| CCNA2 | DA748568 | Homo sapiens | cyclin A2(CCNA2) |
| DNAJC3 | BC047936 | Homo sapiens | DnaJ heat shock protein family (Hsp40) member C3(DNAJC3) |
| DNAJC3 | XM_011521104 | Homo sapiens | DnaJ heat shock protein family (Hsp40) member C3(DNAJC3) |
| DNAJC3 | AA430108 | Homo sapiens | DnaJ heat shock protein family (Hsp40) member C3(DNAJC3) |
| DNAJC3 | XM_011521105 | Homo sapiens | DnaJ heat shock protein family (Hsp40) member C3(DNAJC3) |
| DNAJC3 | AF339773 | Homo sapiens | DnaJ heat shock protein family (Hsp40) member C3(DNAJC3) |
| DNAJC3 | AK024941 | Homo sapiens | DnaJ heat shock protein family (Hsp40) member C3(DNAJC3) |
| DNAJC3 | XM_017020674 | Homo sapiens | DnaJ heat shock protein family (Hsp40) member C3(DNAJC3) |
| DNAJC3 | XM_017020675 | Homo sapiens | DnaJ heat shock protein family (Hsp40) member C3(DNAJC3) |
| DNAJC3 | BC033823 | Homo sapiens | DnaJ heat shock protein family (Hsp40) member C3(DNAJC3) |
| DNAJC3 | AF339835 | Homo sapiens | DnaJ heat shock protein family (Hsp40) member C3(DNAJC3) |
| DNAJC3 | AK292947 | Homo sapiens | DnaJ heat shock protein family (Hsp40) member C3(DNAJC3) |
| DNAJC3 | AK312861 | Homo sapiens | DnaJ heat shock protein family (Hsp40) member C3(DNAJC3) |
| DNAJC3 | NM_006260 | Homo sapiens | DnaJ heat shock protein family (Hsp40) member C3(DNAJC3) |
| ACTA1 | BC012597 | Homo sapiens | actin, alpha 1, skeletal muscle(ACTA1) |
| ACTA1 | CR541796 | Homo sapiens | actin, alpha 1, skeletal muscle(ACTA1) |
| ACTA1 | DQ896197 | Homo sapiens | actin, alpha 1, skeletal muscle(ACTA1) |
| ACTA1 | AY280960 | Homo sapiens | actin, alpha 1, skeletal muscle(ACTA1) |
| ACTA1 | AL598491 | Homo sapiens | actin, alpha 1, skeletal muscle(ACTA1) |
| ACTA1 | CR536516 | Homo sapiens | actin, alpha 1, skeletal muscle(ACTA1) |
| ACTA1 | DQ892949 | Homo sapiens | actin, alpha 1, skeletal muscle(ACTA1) |
| ACTA1 | NM_001100 | Homo sapiens | actin, alpha 1, skeletal muscle(ACTA1) |
| ACTA1 | J00068 | Homo sapiens | actin, alpha 1, skeletal muscle(ACTA1) |
| ACTA1 | AK300697 | Homo sapiens | actin, alpha 1, skeletal muscle(ACTA1) |
| ACTA1 | BX648545 | Homo sapiens | actin, alpha 1, skeletal muscle(ACTA1) |
| ACTA1 | AK096902 | Homo sapiens | actin, alpha 1, skeletal muscle(ACTA1) |
| ASPM | AY367065 | Homo sapiens | abnormal spindle microtubule assembly(ASPM) |
| ASPM | AY099892 | Homo sapiens | abnormal spindle microtubule assembly(ASPM) |
| ASPM | BC040439 | Homo sapiens | abnormal spindle microtubule assembly(ASPM) |
| ASPM | NM_018136 | Homo sapiens | abnormal spindle microtubule assembly(ASPM) |
| ASPM | AY099891 | Homo sapiens | abnormal spindle microtubule assembly(ASPM) |
| ASPM | AY971956 | Homo sapiens | abnormal spindle microtubule assembly(ASPM) |
| ASPM | AK001380 | Homo sapiens | abnormal spindle microtubule assembly(ASPM) |
| ASPM | AY099893 | Homo sapiens | abnormal spindle microtubule assembly(ASPM) |
| ASPM | AY971955 | Homo sapiens | abnormal spindle microtubule assembly(ASPM) |
| ASPM | AY099890 | Homo sapiens | abnormal spindle microtubule assembly(ASPM) |
| ASPM | NM_001206846 | Homo sapiens | abnormal spindle microtubule assembly(ASPM) |
| ASPM | AK095892 | Homo sapiens | abnormal spindle microtubule assembly(ASPM) |
| ASPM | BF970006 | Homo sapiens | abnormal spindle microtubule assembly(ASPM) |
| ASPM | AK001379 | Homo sapiens | abnormal spindle microtubule assembly(ASPM) |
| ASPM | AK226178 | Homo sapiens | abnormal spindle microtubule assembly(ASPM) |
| ASPM | AF509326 | Homo sapiens | abnormal spindle microtubule assembly(ASPM) |
| ASPM | AK001411 | Homo sapiens | abnormal spindle microtubule assembly(ASPM) |
| ASPM | BC034607 | Homo sapiens | abnormal spindle microtubule assembly(ASPM) |
| ASPM | AY101201 | Homo sapiens | abnormal spindle microtubule assembly(ASPM) |
| ASPM | BX648804 | Homo sapiens | abnormal spindle microtubule assembly(ASPM) |
| ASPM | AK125107 | Homo sapiens | abnormal spindle microtubule assembly(ASPM) |
| ASPM | AY971957 | Homo sapiens | abnormal spindle microtubule assembly(ASPM) |
| ASPM | BC015396 | Homo sapiens | abnormal spindle microtubule assembly(ASPM) |
| DCT | NM_001922 | Homo sapiens | dopachrome tautomerase(DCT) |
| DCT | NM_001322182 | Homo sapiens | dopachrome tautomerase(DCT) |
| DCT | NM_001322183 | Homo sapiens | dopachrome tautomerase(DCT) |
| DCT | DQ891466 | Homo sapiens | dopachrome tautomerase(DCT) |
| DCT | NM_001322184 | Homo sapiens | dopachrome tautomerase(DCT) |
| DCT | NM_001322185 | Homo sapiens | dopachrome tautomerase(DCT) |
| DCT | XM_011521049 | Homo sapiens | dopachrome tautomerase(DCT) |
| DCT | NM_001322186 | Homo sapiens | dopachrome tautomerase(DCT) |
| DCT | DQ894649 | Homo sapiens | dopachrome tautomerase(DCT) |
| DCT | DQ902581 | Homo sapiens | dopachrome tautomerase(DCT) |
| DCT | AJ000503 | Homo sapiens | dopachrome tautomerase(DCT) |
| DCT | AJ132932 | Homo sapiens | dopachrome tautomerase(DCT) |
| DCT | BC028311 | Homo sapiens | dopachrome tautomerase(DCT) |
| DCT | AJ132933 | Homo sapiens | dopachrome tautomerase(DCT) |
| DCT | S69231 | Homo sapiens | dopachrome tautomerase(DCT) |
| DCT | NM_001129889 | Homo sapiens | dopachrome tautomerase(DCT) |
| DCT | XM_017020401 | Homo sapiens | dopachrome tautomerase(DCT) |
| DCT | BC104476 | Homo sapiens | dopachrome tautomerase(DCT) |
| DCT | BP221841 | Homo sapiens | dopachrome tautomerase(DCT) |
| DCT | L18967 | Homo sapiens | dopachrome tautomerase(DCT) |
| DCT | D17547 | Homo sapiens | dopachrome tautomerase(DCT) |
| DCT | AF339808 | Homo sapiens | dopachrome tautomerase(DCT) |
| DCT | AK293115 | Homo sapiens | dopachrome tautomerase(DCT) |
| PAX7 | DQ322591 | Homo sapiens | paired box 7(PAX7) |
| PAX7 | NM_001135254 | Homo sapiens | paired box 7(PAX7) |
| PAX7 | BC121165 | Homo sapiens | paired box 7(PAX7) |
| PAX7 | BC121166 | Homo sapiens | paired box 7(PAX7) |
| PAX7 | X96743 | Homo sapiens | paired box 7(PAX7) |
| PAX7 | NM_002584 | Homo sapiens | paired box 7(PAX7) |
| PAX7 | AY578141 | Homo sapiens | paired box 7(PAX7) |
| PAX7 | Z35141 | Homo sapiens | paired box 7(PAX7) |
| PAX7 | AK094779 | Homo sapiens | paired box 7(PAX7) |
| PAX7 | DB474828 | Homo sapiens | paired box 7(PAX7) |
| PAX7 | NM_013945 | Homo sapiens | paired box 7(PAX7) |
| KIFC1 | AK297378 | Homo sapiens | kinesin family member C1(KIFC1) |
| KIFC1 | BC063567 | Homo sapiens | kinesin family member C1(KIFC1) |
| KIFC1 | D14678 | Homo sapiens | kinesin family member C1(KIFC1) |
| KIFC1 | XM_017010836 | Homo sapiens | kinesin family member C1(KIFC1) |
| KIFC1 | XM_017010837 | Homo sapiens | kinesin family member C1(KIFC1) |
| KIFC1 | AK303239 | Homo sapiens | kinesin family member C1(KIFC1) |
| KIFC1 | AI990093 | Homo sapiens | kinesin family member C1(KIFC1) |
| KIFC1 | NM_002263 | Homo sapiens | kinesin family member C1(KIFC1) |
| KIFC1 | XM_011514587 | Homo sapiens | kinesin family member C1(KIFC1) |
| KIFC1 | XM_011514585 | Homo sapiens | kinesin family member C1(KIFC1) |
| KIFC1 | BC073878 | Homo sapiens | kinesin family member C1(KIFC1) |
| KIFC1 | BC121041 | Homo sapiens | kinesin family member C1(KIFC1) |
| KIFC1 | AJ010479 | Homo sapiens | kinesin family member C1(KIFC1) |
| KIFC1 | BC000712 | Homo sapiens | kinesin family member C1(KIFC1) |
| KIFC1 | BC121042 | Homo sapiens | kinesin family member C1(KIFC1) |
| KIFC1 | BC098438 | Homo sapiens | kinesin family member C1(KIFC1) |
| KIFC1 | BE885900 | Homo sapiens | kinesin family member C1(KIFC1) |
| PLSCR5 | XM_017006373 | Homo sapiens | phospholipid scramblase family member 5(PLSCR5) |
| PLSCR5 | BC157886 | Homo sapiens | phospholipid scramblase family member 5(PLSCR5) |
| PLSCR5 | NM_001085420 | Homo sapiens | phospholipid scramblase family member 5(PLSCR5) |
| PLSCR5 | NM_001321245 | Homo sapiens | phospholipid scramblase family member 5(PLSCR5) |
| PLSCR5 | AY436642 | Homo sapiens | phospholipid scramblase family member 5(PLSCR5) |
| PLSCR5 | BX431427 | Homo sapiens | phospholipid scramblase family member 5(PLSCR5) |
| PLSCR5 | BC171917 | Homo sapiens | phospholipid scramblase family member 5(PLSCR5) |
| MYOD1 | AI300241 | Homo sapiens | myogenic differentiation 1(MYOD1) |
| MYOD1 | BT007157 | Homo sapiens | myogenic differentiation 1(MYOD1) |
| MYOD1 | X56677 | Homo sapiens | myogenic differentiation 1(MYOD1) |
| MYOD1 | BF304059 | Homo sapiens | myogenic differentiation 1(MYOD1) |
| MYOD1 | X17650 | Homo sapiens | myogenic differentiation 1(MYOD1) |
| MYOD1 | BC064493 | Homo sapiens | myogenic differentiation 1(MYOD1) |
| MYOD1 | BF205042 | Homo sapiens | myogenic differentiation 1(MYOD1) |
| MYOD1 | NM_002478 | Homo sapiens | myogenic differentiation 1(MYOD1) |
| MYOD1 | AK314968 | Homo sapiens | myogenic differentiation 1(MYOD1) |
| TNNT2 | AJ709280 | Homo sapiens | troponin T2, cardiac type(TNNT2) |
| TNNT2 | X74819 | Homo sapiens | troponin T2, cardiac type(TNNT2) |
| TNNT2 | L40162 | Homo sapiens | troponin T2, cardiac type(TNNT2) |
| TNNT2 | NM_001276345 | Homo sapiens | troponin T2, cardiac type(TNNT2) |
| TNNT2 | XM_011509946 | Homo sapiens | troponin T2, cardiac type(TNNT2) |
| TNNT2 | AL832707 | Homo sapiens | troponin T2, cardiac type(TNNT2) |
| TNNT2 | NM_001276346 | Homo sapiens | troponin T2, cardiac type(TNNT2) |
| TNNT2 | NM_001276347 | Homo sapiens | troponin T2, cardiac type(TNNT2) |
| TNNT2 | XM_011509944 | Homo sapiens | troponin T2, cardiac type(TNNT2) |
| TNNT2 | XM_011509943 | Homo sapiens | troponin T2, cardiac type(TNNT2) |
| TNNT2 | BC002653 | Homo sapiens | troponin T2, cardiac type(TNNT2) |
| TNNT2 | X79855 | Homo sapiens | troponin T2, cardiac type(TNNT2) |
| TNNT2 | X79856 | Homo sapiens | troponin T2, cardiac type(TNNT2) |
| TNNT2 | X83744 | Homo sapiens | troponin T2, cardiac type(TNNT2) |
| TNNT2 | X83743 | Homo sapiens | troponin T2, cardiac type(TNNT2) |
| TNNT2 | XM_017002216 | Homo sapiens | troponin T2, cardiac type(TNNT2) |
| TNNT2 | HQ447413 | Homo sapiens | troponin T2, cardiac type(TNNT2) |
| TNNT2 | XM_017002217 | Homo sapiens | troponin T2, cardiac type(TNNT2) |
| TNNT2 | AK309493 | Homo sapiens | troponin T2, cardiac type(TNNT2) |
| TNNT2 | NM_001001430 | Homo sapiens | troponin T2, cardiac type(TNNT2) |
| TNNT2 | AK125236 | Homo sapiens | troponin T2, cardiac type(TNNT2) |
| TNNT2 | AK290621 | Homo sapiens | troponin T2, cardiac type(TNNT2) |
| TNNT2 | AY277394 | Homo sapiens | troponin T2, cardiac type(TNNT2) |
| TNNT2 | NM_001001432 | Homo sapiens | troponin T2, cardiac type(TNNT2) |
| TNNT2 | NM_001001431 | Homo sapiens | troponin T2, cardiac type(TNNT2) |
| TNNT2 | AK055533 | Homo sapiens | troponin T2, cardiac type(TNNT2) |
| TNNT2 | AA865519 | Homo sapiens | troponin T2, cardiac type(TNNT2) |
| TNNT2 | NM_000364 | Homo sapiens | troponin T2, cardiac type(TNNT2) |
| TNNT2 | S71125 | Homo sapiens | troponin T2, cardiac type(TNNT2) |
| TNNT2 | S71126 | Homo sapiens | troponin T2, cardiac type(TNNT2) |
| TNNT2 | XM_011509939 | Homo sapiens | troponin T2, cardiac type(TNNT2) |
| TNNT2 | X79858 | Homo sapiens | troponin T2, cardiac type(TNNT2) |
| TNNT2 | XM_011509938 | Homo sapiens | troponin T2, cardiac type(TNNT2) |
| TNNT2 | X79859 | Homo sapiens | troponin T2, cardiac type(TNNT2) |
| TNNT2 | XM_006711509 | Homo sapiens | troponin T2, cardiac type(TNNT2) |
| TNNT2 | XM_006711508 | Homo sapiens | troponin T2, cardiac type(TNNT2) |
| TNNT2 | S64668 | Homo sapiens | troponin T2, cardiac type(TNNT2) |
| TNNT2 | XM_011509942 | Homo sapiens | troponin T2, cardiac type(TNNT2) |
| TNNT2 | X79861 | Homo sapiens | troponin T2, cardiac type(TNNT2) |
| TNNT2 | XM_011509941 | Homo sapiens | troponin T2, cardiac type(TNNT2) |
| TNNT2 | XM_011509940 | Homo sapiens | troponin T2, cardiac type(TNNT2) |
| DCX | AK299822 | Homo sapiens | doublecortin(DCX) |
| DCX | NM_000555 | Homo sapiens | doublecortin(DCX) |
| DCX | AK002120 | Homo sapiens | doublecortin(DCX) |
| DCX | AK297297 | Homo sapiens | doublecortin(DCX) |
| DCX | AU120758 | Homo sapiens | doublecortin(DCX) |
| DCX | XM_017029312 | Homo sapiens | doublecortin(DCX) |
| DCX | DC375659 | Homo sapiens | doublecortin(DCX) |
| DCX | HQ447825 | Homo sapiens | doublecortin(DCX) |
| DCX | NM_001195553 | Homo sapiens | doublecortin(DCX) |
| DCX | XM_011530880 | Homo sapiens | doublecortin(DCX) |
| DCX | AF040254 | Homo sapiens | doublecortin(DCX) |
| DCX | AJ003112 | Homo sapiens | doublecortin(DCX) |
| DCX | AF040255 | Homo sapiens | doublecortin(DCX) |
| DCX | NM_178151 | Homo sapiens | doublecortin(DCX) |
| DCX | AW163329 | Homo sapiens | doublecortin(DCX) |
| DCX | BC027925 | Homo sapiens | doublecortin(DCX) |
| DCX | AF034634 | Homo sapiens | doublecortin(DCX) |
| DCX | DA497601 | Homo sapiens | doublecortin(DCX) |
| DCX | XM_011530878 | Homo sapiens | doublecortin(DCX) |
| DCX | AK290455 | Homo sapiens | doublecortin(DCX) |
| DCX | NM_178153 | Homo sapiens | doublecortin(DCX) |
| DCX | NM_178152 | Homo sapiens | doublecortin(DCX) |
| DCX | XM_011530879 | Homo sapiens | doublecortin(DCX) |
| PMP2 | AK311758 | Homo sapiens | peripheral myelin protein 2(PMP2) |
| PMP2 | X62167 | Homo sapiens | peripheral myelin protein 2(PMP2) |
| PMP2 | BC034997 | Homo sapiens | peripheral myelin protein 2(PMP2) |
| PMP2 | CF455669 | Homo sapiens | peripheral myelin protein 2(PMP2) |
| PMP2 | CR541649 | Homo sapiens | peripheral myelin protein 2(PMP2) |
| PMP2 | DQ891506 | Homo sapiens | peripheral myelin protein 2(PMP2) |
| PMP2 | CR541738 | Homo sapiens | peripheral myelin protein 2(PMP2) |
| PMP2 | DQ891507 | Homo sapiens | peripheral myelin protein 2(PMP2) |
| PMP2 | NM_002677 | Homo sapiens | peripheral myelin protein 2(PMP2) |
| PMP2 | AK307118 | Homo sapiens | peripheral myelin protein 2(PMP2) |
| PMP2 | DQ894697 | Homo sapiens | peripheral myelin protein 2(PMP2) |
| PMP2 | DQ786226 | Homo sapiens | peripheral myelin protein 2(PMP2) |
| MAPT | AK226139 | Homo sapiens | microtubule associated protein tau(MAPT) |
| MAPT | BC061892 | Homo sapiens | microtubule associated protein tau(MAPT) |
| MAPT | AK299658 | Homo sapiens | microtubule associated protein tau(MAPT) |
| MAPT | BI552187 | Homo sapiens | microtubule associated protein tau(MAPT) |
| MAPT | AW295014 | Homo sapiens | microtubule associated protein tau(MAPT) |
| MAPT | BC101936 | Homo sapiens | microtubule associated protein tau(MAPT) |
| MAPT | BN000503 | Homo sapiens | microtubule associated protein tau(MAPT) |
| MAPT | X14474 | Homo sapiens | microtubule associated protein tau(MAPT) |
| MAPT | AW161357 | Homo sapiens | microtubule associated protein tau(MAPT) |
| MAPT | BC098281 | Homo sapiens | microtubule associated protein tau(MAPT) |
| MAPT | FJ429137 | Homo sapiens | microtubule associated protein tau(MAPT) |
| MAPT | XM_005257370 | Homo sapiens | microtubule associated protein tau(MAPT) |
| MAPT | XM_005257371 | Homo sapiens | microtubule associated protein tau(MAPT) |
| MAPT | NM_001123067 | Homo sapiens | microtubule associated protein tau(MAPT) |
| MAPT | NM_001123066 | Homo sapiens | microtubule associated protein tau(MAPT) |
| MAPT | BC114504 | Homo sapiens | microtubule associated protein tau(MAPT) |
| MAPT | BT006772 | Homo sapiens | microtubule associated protein tau(MAPT) |
| MAPT | BC099721 | Homo sapiens | microtubule associated protein tau(MAPT) |
| MAPT | BC071830 | Homo sapiens | microtubule associated protein tau(MAPT) |
| MAPT | BC000558 | Homo sapiens | microtubule associated protein tau(MAPT) |
| MAPT | AK095802 | Homo sapiens | microtubule associated protein tau(MAPT) |
| MAPT | NM_016834 | Homo sapiens | microtubule associated protein tau(MAPT) |
| MAPT | AK055986 | Homo sapiens | microtubule associated protein tau(MAPT) |
| MAPT | BC114948 | Homo sapiens | microtubule associated protein tau(MAPT) |
| MAPT | NM_016835 | Homo sapiens | microtubule associated protein tau(MAPT) |
| MAPT | BC094805 | Homo sapiens | microtubule associated protein tau(MAPT) |
| MAPT | XM_005257366 | Homo sapiens | microtubule associated protein tau(MAPT) |
| MAPT | XM_005257367 | Homo sapiens | microtubule associated protein tau(MAPT) |
| MAPT | BC040444 | Homo sapiens | microtubule associated protein tau(MAPT) |
| MAPT | DR002467 | Homo sapiens | microtubule associated protein tau(MAPT) |
| MAPT | XM_005257364 | Homo sapiens | microtubule associated protein tau(MAPT) |
| MAPT | AK299704 | Homo sapiens | microtubule associated protein tau(MAPT) |
| MAPT | XM_005257365 | Homo sapiens | microtubule associated protein tau(MAPT) |
| MAPT | AF456477 | Homo sapiens | microtubule associated protein tau(MAPT) |
| MAPT | XM_005257368 | Homo sapiens | microtubule associated protein tau(MAPT) |
| MAPT | XM_005257369 | Homo sapiens | microtubule associated protein tau(MAPT) |
| MAPT | AB073354 | Homo sapiens | microtubule associated protein tau(MAPT) |
| MAPT | AY730549 | Homo sapiens | microtubule associated protein tau(MAPT) |
| MAPT | J03778 | Homo sapiens | microtubule associated protein tau(MAPT) |
| MAPT | NM_005910 | Homo sapiens | microtubule associated protein tau(MAPT) |
| MAPT | XM_005257362 | Homo sapiens | microtubule associated protein tau(MAPT) |
| MAPT | AY526356 | Homo sapiens | microtubule associated protein tau(MAPT) |
| MAPT | NM_001203251 | Homo sapiens | microtubule associated protein tau(MAPT) |
| MAPT | NM_001203252 | Homo sapiens | microtubule associated protein tau(MAPT) |
| MAPT | NM_016841 | Homo sapiens | microtubule associated protein tau(MAPT) |
| MAPT | DN996935 | Homo sapiens | microtubule associated protein tau(MAPT) |
| MAPT | BC032572 | Homo sapiens | microtubule associated protein tau(MAPT) |
| MAPT | M25298 | Homo sapiens | microtubule associated protein tau(MAPT) |
| CFAP54 | XM_011539081 | Homo sapiens | cilia and flagella associated protein 54(CFAP54) |
| CFAP54 | DN831445 | Homo sapiens | cilia and flagella associated protein 54(CFAP54) |
| CFAP54 | XM_011539084 | Homo sapiens | cilia and flagella associated protein 54(CFAP54) |
| CFAP54 | XM_011539083 | Homo sapiens | cilia and flagella associated protein 54(CFAP54) |
| CFAP54 | AK056076 | Homo sapiens | cilia and flagella associated protein 54(CFAP54) |
| CFAP54 | BC148603 | Homo sapiens | cilia and flagella associated protein 54(CFAP54) |
| CFAP54 | BC041426 | Homo sapiens | cilia and flagella associated protein 54(CFAP54) |
| CFAP54 | NM_001306084 | Homo sapiens | cilia and flagella associated protein 54(CFAP54) |
| CFAP54 | XR_945241 | Homo sapiens | cilia and flagella associated protein 54(CFAP54) |
| CFAP54 | XM_011539069 | Homo sapiens | cilia and flagella associated protein 54(CFAP54) |
| CFAP54 | XR_945242 | Homo sapiens | cilia and flagella associated protein 54(CFAP54) |
| CFAP54 | XR_945243 | Homo sapiens | cilia and flagella associated protein 54(CFAP54) |
| CFAP54 | AK307646 | Homo sapiens | cilia and flagella associated protein 54(CFAP54) |
| CFAP54 | XM_011539085 | Homo sapiens | cilia and flagella associated protein 54(CFAP54) |
| CFAP54 | AK126100 | Homo sapiens | cilia and flagella associated protein 54(CFAP54) |
| CFAP54 | BX644506 | Homo sapiens | cilia and flagella associated protein 54(CFAP54) |
| CFAP54 | AK091789 | Homo sapiens | cilia and flagella associated protein 54(CFAP54) |
| CFAP54 | DV080428 | Homo sapiens | cilia and flagella associated protein 54(CFAP54) |
| CFAP54 | DV080725 | Homo sapiens | cilia and flagella associated protein 54(CFAP54) |
| CFAP54 | XM_011539071 | Homo sapiens | cilia and flagella associated protein 54(CFAP54) |
| CFAP54 | XM_011539070 | Homo sapiens | cilia and flagella associated protein 54(CFAP54) |
| CFAP54 | DV080224 | Homo sapiens | cilia and flagella associated protein 54(CFAP54) |
| CFAP54 | XM_011539073 | Homo sapiens | cilia and flagella associated protein 54(CFAP54) |
| CFAP54 | XM_011539072 | Homo sapiens | cilia and flagella associated protein 54(CFAP54) |
| CFAP54 | CF887548 | Homo sapiens | cilia and flagella associated protein 54(CFAP54) |
| CFAP54 | XM_011539079 | Homo sapiens | cilia and flagella associated protein 54(CFAP54) |
| CFAP54 | DB225896 | Homo sapiens | cilia and flagella associated protein 54(CFAP54) |
| CFAP54 | XM_011539078 | Homo sapiens | cilia and flagella associated protein 54(CFAP54) |
| CFAP54 | BC153167 | Homo sapiens | cilia and flagella associated protein 54(CFAP54) |
| CFAP54 | BX281772 | Homo sapiens | cilia and flagella associated protein 54(CFAP54) |
| CFAP54 | XM_011539074 | Homo sapiens | cilia and flagella associated protein 54(CFAP54) |
| CFAP54 | XM_011539077 | Homo sapiens | cilia and flagella associated protein 54(CFAP54) |
| CFAP54 | XM_011539076 | Homo sapiens | cilia and flagella associated protein 54(CFAP54) |
| CFAP54 | CF891050 | Homo sapiens | cilia and flagella associated protein 54(CFAP54) |
| CFAP54 | EG328110 | Homo sapiens | cilia and flagella associated protein 54(CFAP54) |
| CFAP54 | XM_017018867 | Homo sapiens | cilia and flagella associated protein 54(CFAP54) |
| CFAP54 | XM_017018866 | Homo sapiens | cilia and flagella associated protein 54(CFAP54) |
| CFAP54 | NM_198520 | Homo sapiens | cilia and flagella associated protein 54(CFAP54) |
| UGT2B7 | DQ893657 | Homo sapiens | UDP glucuronosyltransferase family 2 member B7(UGT2B7) |
| UGT2B7 | DQ895814 | Homo sapiens | UDP glucuronosyltransferase family 2 member B7(UGT2B7) |
| UGT2B7 | XM_005265702 | Homo sapiens | UDP glucuronosyltransferase family 2 member B7(UGT2B7) |
| UGT2B7 | J05428 | Homo sapiens | UDP glucuronosyltransferase family 2 member B7(UGT2B7) |
| UGT2B7 | AW614236 | Homo sapiens | UDP glucuronosyltransferase family 2 member B7(UGT2B7) |
| UGT2B7 | S82485 | Homo sapiens | UDP glucuronosyltransferase family 2 member B7(UGT2B7) |
| UGT2B7 | AK223142 | Homo sapiens | UDP glucuronosyltransferase family 2 member B7(UGT2B7) |
| UGT2B7 | AK313190 | Homo sapiens | UDP glucuronosyltransferase family 2 member B7(UGT2B7) |
| UGT2B7 | XM_011532230 | Homo sapiens | UDP glucuronosyltransferase family 2 member B7(UGT2B7) |
| UGT2B7 | NM_001074 | Homo sapiens | UDP glucuronosyltransferase family 2 member B7(UGT2B7) |
| UGT2B7 | NM_001330719 | Homo sapiens | UDP glucuronosyltransferase family 2 member B7(UGT2B7) |
| UGT2B7 | BC030974 | Homo sapiens | UDP glucuronosyltransferase family 2 member B7(UGT2B7) |
| UGT2B7 | XM_011532229 | Homo sapiens | UDP glucuronosyltransferase family 2 member B7(UGT2B7) |
| RBM47 | XM_017008310 | Homo sapiens | RNA binding motif protein 47(RBM47) |
| RBM47 | CB124502 | Homo sapiens | RNA binding motif protein 47(RBM47) |
| RBM47 | AK025296 | Homo sapiens | RNA binding motif protein 47(RBM47) |
| RBM47 | HQ258329 | Homo sapiens | RNA binding motif protein 47(RBM47) |
| RBM47 | DA861151 | Homo sapiens | RNA binding motif protein 47(RBM47) |
| RBM47 | BC034402 | Homo sapiens | RNA binding motif protein 47(RBM47) |
| RBM47 | XM_011513708 | Homo sapiens | RNA binding motif protein 47(RBM47) |
| RBM47 | BX648404 | Homo sapiens | RNA binding motif protein 47(RBM47) |
| RBM47 | XM_011513707 | Homo sapiens | RNA binding motif protein 47(RBM47) |
| RBM47 | XM_011513704 | Homo sapiens | RNA binding motif protein 47(RBM47) |
| RBM47 | BC126261 | Homo sapiens | RNA binding motif protein 47(RBM47) |
| RBM47 | BP332501 | Homo sapiens | RNA binding motif protein 47(RBM47) |
| RBM47 | BM837005 | Homo sapiens | RNA binding motif protein 47(RBM47) |
| RBM47 | AK000280 | Homo sapiens | RNA binding motif protein 47(RBM47) |
| RBM47 | NM_019027 | Homo sapiens | RNA binding motif protein 47(RBM47) |
| RBM47 | AF262323 | Homo sapiens | RNA binding motif protein 47(RBM47) |
| RBM47 | AK304206 | Homo sapiens | RNA binding motif protein 47(RBM47) |
| RBM47 | XM_017008308 | Homo sapiens | RNA binding motif protein 47(RBM47) |
| RBM47 | BC143942 | Homo sapiens | RNA binding motif protein 47(RBM47) |
| RBM47 | XM_017008307 | Homo sapiens | RNA binding motif protein 47(RBM47) |
| RBM47 | AK057152 | Homo sapiens | RNA binding motif protein 47(RBM47) |
| RBM47 | XM_017008306 | Homo sapiens | RNA binding motif protein 47(RBM47) |
| RBM47 | XM_017008305 | Homo sapiens | RNA binding motif protein 47(RBM47) |
| RBM47 | XM_017008304 | Homo sapiens | RNA binding motif protein 47(RBM47) |
| RBM47 | AK024997 | Homo sapiens | RNA binding motif protein 47(RBM47) |
| RBM47 | XM_005248107 | Homo sapiens | RNA binding motif protein 47(RBM47) |
| RBM47 | XM_005248108 | Homo sapiens | RNA binding motif protein 47(RBM47) |
| RBM47 | XM_005248109 | Homo sapiens | RNA binding motif protein 47(RBM47) |
| RBM47 | XM_005248103 | Homo sapiens | RNA binding motif protein 47(RBM47) |
| RBM47 | NM_001098634 | Homo sapiens | RNA binding motif protein 47(RBM47) |
| RBM47 | XM_017008309 | Homo sapiens | RNA binding motif protein 47(RBM47) |
| RBM47 | BC071585 | Homo sapiens | RNA binding motif protein 47(RBM47) |
| DNAH7 | Z83801 | Homo sapiens | dynein axonemal heavy chain 7(DNAH7) |
| DNAH7 | NM_018897 | Homo sapiens | dynein axonemal heavy chain 7(DNAH7) |
| DNAH7 | XM_011511491 | Homo sapiens | dynein axonemal heavy chain 7(DNAH7) |
| DNAH7 | XM_011511490 | Homo sapiens | dynein axonemal heavy chain 7(DNAH7) |
| DNAH7 | XM_011511493 | Homo sapiens | dynein axonemal heavy chain 7(DNAH7) |
| DNAH7 | BP199399 | Homo sapiens | dynein axonemal heavy chain 7(DNAH7) |
| DNAH7 | XM_011511492 | Homo sapiens | dynein axonemal heavy chain 7(DNAH7) |
| DNAH7 | BX117789 | Homo sapiens | dynein axonemal heavy chain 7(DNAH7) |
| DNAH7 | XM_011511488 | Homo sapiens | dynein axonemal heavy chain 7(DNAH7) |
| DNAH7 | XM_011511487 | Homo sapiens | dynein axonemal heavy chain 7(DNAH7) |
| DNAH7 | BC029567 | Homo sapiens | dynein axonemal heavy chain 7(DNAH7) |
| DNAH7 | XM_011511489 | Homo sapiens | dynein axonemal heavy chain 7(DNAH7) |
| DNAH7 | AB023161 | Homo sapiens | dynein axonemal heavy chain 7(DNAH7) |
| DNAH7 | XM_011511495 | Homo sapiens | dynein axonemal heavy chain 7(DNAH7) |
| DNAH7 | XM_011511494 | Homo sapiens | dynein axonemal heavy chain 7(DNAH7) |
| DNAH7 | XR_922968 | Homo sapiens | dynein axonemal heavy chain 7(DNAH7) |
| DNAH7 | XM_011511497 | Homo sapiens | dynein axonemal heavy chain 7(DNAH7) |
| DNAH7 | XM_017004504 | Homo sapiens | dynein axonemal heavy chain 7(DNAH7) |
| DNAH7 | AJ132084 | Homo sapiens | dynein axonemal heavy chain 7(DNAH7) |
| DNAH7 | CR749651 | Homo sapiens | dynein axonemal heavy chain 7(DNAH7) |
| DNAH7 | AK094515 | Homo sapiens | dynein axonemal heavy chain 7(DNAH7) |
| DNAH7 | AF327442 | Homo sapiens | dynein axonemal heavy chain 7(DNAH7) |
| COL11A1 | J04177 | Homo sapiens | collagen type XI alpha 1 chain(COL11A1) |
| COL11A1 | AK299025 | Homo sapiens | collagen type XI alpha 1 chain(COL11A1) |
| COL11A1 | AU118365 | Homo sapiens | collagen type XI alpha 1 chain(COL11A1) |
| COL11A1 | BC117697 | Homo sapiens | collagen type XI alpha 1 chain(COL11A1) |
| COL11A1 | AB208844 | Homo sapiens | collagen type XI alpha 1 chain(COL11A1) |
| COL11A1 | NM_001190709 | Homo sapiens | collagen type XI alpha 1 chain(COL11A1) |
| COL11A1 | NM_080629 | Homo sapiens | collagen type XI alpha 1 chain(COL11A1) |
| COL11A1 | NM_001854 | Homo sapiens | collagen type XI alpha 1 chain(COL11A1) |
| COL11A1 | BU159588 | Homo sapiens | collagen type XI alpha 1 chain(COL11A1) |
| COL11A1 | NM_080630 | Homo sapiens | collagen type XI alpha 1 chain(COL11A1) |
| COL11A1 | BX476821 | Homo sapiens | collagen type XI alpha 1 chain(COL11A1) |
| COL11A1 | NR_134980 | Homo sapiens | collagen type XI alpha 1 chain(COL11A1) |
| COL11A1 | XM_017000334 | Homo sapiens | collagen type XI alpha 1 chain(COL11A1) |
| COL11A1 | XM_017000335 | Homo sapiens | collagen type XI alpha 1 chain(COL11A1) |
| COL11A1 | XM_017000336 | Homo sapiens | collagen type XI alpha 1 chain(COL11A1) |
| COL11A1 | CA448616 | Homo sapiens | collagen type XI alpha 1 chain(COL11A1) |
| COL11A1 | XM_017000337 | Homo sapiens | collagen type XI alpha 1 chain(COL11A1) |
| LAMA4 | AK027151 | Homo sapiens | laminin subunit alpha 4(LAMA4) |
| LAMA4 | X76939 | Homo sapiens | laminin subunit alpha 4(LAMA4) |
| LAMA4 | U77706 | Homo sapiens | laminin subunit alpha 4(LAMA4) |
| LAMA4 | AB210027 | Homo sapiens | laminin subunit alpha 4(LAMA4) |
| LAMA4 | XM_017010854 | Homo sapiens | laminin subunit alpha 4(LAMA4) |
| LAMA4 | S78569 | Homo sapiens | laminin subunit alpha 4(LAMA4) |
| LAMA4 | BC004241 | Homo sapiens | laminin subunit alpha 4(LAMA4) |
| LAMA4 | BM662226 | Homo sapiens | laminin subunit alpha 4(LAMA4) |
| LAMA4 | NM_001105207 | Homo sapiens | laminin subunit alpha 4(LAMA4) |
| LAMA4 | NM_001105208 | Homo sapiens | laminin subunit alpha 4(LAMA4) |
| LAMA4 | NM_001105209 | Homo sapiens | laminin subunit alpha 4(LAMA4) |
| LAMA4 | BT006690 | Homo sapiens | laminin subunit alpha 4(LAMA4) |
| LAMA4 | NM_001105206 | Homo sapiens | laminin subunit alpha 4(LAMA4) |
| LAMA4 | AJ710266 | Homo sapiens | laminin subunit alpha 4(LAMA4) |
| LAMA4 | XM_005266984 | Homo sapiens | laminin subunit alpha 4(LAMA4) |
| LAMA4 | XM_005266983 | Homo sapiens | laminin subunit alpha 4(LAMA4) |
| LAMA4 | BX648467 | Homo sapiens | laminin subunit alpha 4(LAMA4) |
| LAMA4 | X70904 | Homo sapiens | laminin subunit alpha 4(LAMA4) |
| LAMA4 | BP234809 | Homo sapiens | laminin subunit alpha 4(LAMA4) |
| LAMA4 | BC066552 | Homo sapiens | laminin subunit alpha 4(LAMA4) |
| LAMA4 | BM014298 | Homo sapiens | laminin subunit alpha 4(LAMA4) |
| LAMA4 | JF432726 | Homo sapiens | laminin subunit alpha 4(LAMA4) |
| LAMA4 | AK304401 | Homo sapiens | laminin subunit alpha 4(LAMA4) |
| LAMA4 | CR407622 | Homo sapiens | laminin subunit alpha 4(LAMA4) |
| LAMA4 | XR_001743406 | Homo sapiens | laminin subunit alpha 4(LAMA4) |
| LAMA4 | XR_001743407 | Homo sapiens | laminin subunit alpha 4(LAMA4) |
| LAMA4 | BC026237 | Homo sapiens | laminin subunit alpha 4(LAMA4) |
| LAMA4 | DB271101 | Homo sapiens | laminin subunit alpha 4(LAMA4) |
| LAMA4 | NM_002290 | Homo sapiens | laminin subunit alpha 4(LAMA4) |
| LAMA4 | X91171 | Homo sapiens | laminin subunit alpha 4(LAMA4) |
| NRXN1 | BM021375 | Homo sapiens | neurexin 1(NRXN1) |
| NRXN1 | NM_001330078 | Homo sapiens | neurexin 1(NRXN1) |
| NRXN1 | NM_001330079 | Homo sapiens | neurexin 1(NRXN1) |
| NRXN1 | NM_001330077 | Homo sapiens | neurexin 1(NRXN1) |
| NRXN1 | XM_011533180 | Homo sapiens | neurexin 1(NRXN1) |
| NRXN1 | XM_017005304 | Homo sapiens | neurexin 1(NRXN1) |
| NRXN1 | XM_017005305 | Homo sapiens | neurexin 1(NRXN1) |
| NRXN1 | XM_017005303 | Homo sapiens | neurexin 1(NRXN1) |
| NRXN1 | BC046631 | Homo sapiens | neurexin 1(NRXN1) |
| NRXN1 | XM_011533183 | Homo sapiens | neurexin 1(NRXN1) |
| NRXN1 | XM_011533177 | Homo sapiens | neurexin 1(NRXN1) |
| NRXN1 | XM_011533178 | Homo sapiens | neurexin 1(NRXN1) |
| NRXN1 | XM_011533175 | Homo sapiens | neurexin 1(NRXN1) |
| NRXN1 | XM_017005308 | Homo sapiens | neurexin 1(NRXN1) |
| NRXN1 | AF087975 | Homo sapiens | neurexin 1(NRXN1) |
| NRXN1 | XM_017005309 | Homo sapiens | neurexin 1(NRXN1) |
| NRXN1 | BX647616 | Homo sapiens | neurexin 1(NRXN1) |
| NRXN1 | XM_017005306 | Homo sapiens | neurexin 1(NRXN1) |
| NRXN1 | XM_017005307 | Homo sapiens | neurexin 1(NRXN1) |
| NRXN1 | NM_001320156 | Homo sapiens | neurexin 1(NRXN1) |
| NRXN1 | NM_001320157 | Homo sapiens | neurexin 1(NRXN1) |
| NRXN1 | XM_006712140 | Homo sapiens | neurexin 1(NRXN1) |
| NRXN1 | XM_017005330 | Homo sapiens | neurexin 1(NRXN1) |
| NRXN1 | AB011150 | Homo sapiens | neurexin 1(NRXN1) |
| NRXN1 | BX113737 | Homo sapiens | neurexin 1(NRXN1) |
| NRXN1 | EF539882 | Homo sapiens | neurexin 1(NRXN1) |
| NRXN1 | XM_017005337 | Homo sapiens | neurexin 1(NRXN1) |
| NRXN1 | XM_017005335 | Homo sapiens | neurexin 1(NRXN1) |
| NRXN1 | XM_017005336 | Homo sapiens | neurexin 1(NRXN1) |
| NRXN1 | NM_004801 | Homo sapiens | neurexin 1(NRXN1) |
| NRXN1 | XM_017005333 | Homo sapiens | neurexin 1(NRXN1) |
| NRXN1 | XM_011533174 | Homo sapiens | neurexin 1(NRXN1) |
| NRXN1 | XM_017005334 | Homo sapiens | neurexin 1(NRXN1) |
| NRXN1 | XM_011533171 | Homo sapiens | neurexin 1(NRXN1) |
| NRXN1 | XM_017005331 | Homo sapiens | neurexin 1(NRXN1) |
| NRXN1 | XM_011533172 | Homo sapiens | neurexin 1(NRXN1) |
| NRXN1 | XM_017005332 | Homo sapiens | neurexin 1(NRXN1) |
| NRXN1 | XM_011533167 | Homo sapiens | neurexin 1(NRXN1) |
| NRXN1 | AK295773 | Homo sapiens | neurexin 1(NRXN1) |
| NRXN1 | DA153976 | Homo sapiens | neurexin 1(NRXN1) |
| NRXN1 | AK124726 | Homo sapiens | neurexin 1(NRXN1) |
| NRXN1 | XM_005264643 | Homo sapiens | neurexin 1(NRXN1) |
| NRXN1 | BC150247 | Homo sapiens | neurexin 1(NRXN1) |
| NRXN1 | XM_017005326 | Homo sapiens | neurexin 1(NRXN1) |
| NRXN1 | NM_138735 | Homo sapiens | neurexin 1(NRXN1) |
| NRXN1 | XM_017005327 | Homo sapiens | neurexin 1(NRXN1) |
| NRXN1 | DA168874 | Homo sapiens | neurexin 1(NRXN1) |
| NRXN1 | XM_017005324 | Homo sapiens | neurexin 1(NRXN1) |
| NRXN1 | XM_017005325 | Homo sapiens | neurexin 1(NRXN1) |
| NRXN1 | XM_017005322 | Homo sapiens | neurexin 1(NRXN1) |
| NRXN1 | XM_017005323 | Homo sapiens | neurexin 1(NRXN1) |
| NRXN1 | XM_017005320 | Homo sapiens | neurexin 1(NRXN1) |
| NRXN1 | AK093260 | Homo sapiens | neurexin 1(NRXN1) |
| NRXN1 | XM_017005321 | Homo sapiens | neurexin 1(NRXN1) |
| NRXN1 | C15866 | Homo sapiens | neurexin 1(NRXN1) |
| NRXN1 | NM_001135659 | Homo sapiens | neurexin 1(NRXN1) |
| NRXN1 | XM_017005328 | Homo sapiens | neurexin 1(NRXN1) |
| NRXN1 | DC324848 | Homo sapiens | neurexin 1(NRXN1) |
| NRXN1 | XM_017005329 | Homo sapiens | neurexin 1(NRXN1) |
| NRXN1 | DA320230 | Homo sapiens | neurexin 1(NRXN1) |
| NRXN1 | NM_001330096 | Homo sapiens | neurexin 1(NRXN1) |
| NRXN1 | DA322531 | Homo sapiens | neurexin 1(NRXN1) |
| NRXN1 | NM_001330097 | Homo sapiens | neurexin 1(NRXN1) |
| NRXN1 | XM_005264642 | Homo sapiens | neurexin 1(NRXN1) |
| NRXN1 | NM_001330094 | Homo sapiens | neurexin 1(NRXN1) |
| NRXN1 | NM_001330095 | Homo sapiens | neurexin 1(NRXN1) |
| NRXN1 | NM_001330092 | Homo sapiens | neurexin 1(NRXN1) |
| NRXN1 | NM_001330093 | Homo sapiens | neurexin 1(NRXN1) |
| NRXN1 | NM_001330090 | Homo sapiens | neurexin 1(NRXN1) |
| NRXN1 | NM_001330091 | Homo sapiens | neurexin 1(NRXN1) |
| NRXN1 | NM_001330089 | Homo sapiens | neurexin 1(NRXN1) |
| NRXN1 | BC125179 | Homo sapiens | neurexin 1(NRXN1) |
| NRXN1 | NM_001330087 | Homo sapiens | neurexin 1(NRXN1) |
| NRXN1 | NM_001330088 | Homo sapiens | neurexin 1(NRXN1) |
| NRXN1 | XM_017005315 | Homo sapiens | neurexin 1(NRXN1) |
| NRXN1 | XM_017005316 | Homo sapiens | neurexin 1(NRXN1) |
| NRXN1 | XM_017005313 | Homo sapiens | neurexin 1(NRXN1) |
| NRXN1 | XM_017005314 | Homo sapiens | neurexin 1(NRXN1) |
| NRXN1 | XM_017005311 | Homo sapiens | neurexin 1(NRXN1) |
| NRXN1 | XM_017005312 | Homo sapiens | neurexin 1(NRXN1) |
| NRXN1 | XM_017005310 | Homo sapiens | neurexin 1(NRXN1) |
| NRXN1 | BC125180 | Homo sapiens | neurexin 1(NRXN1) |
| NRXN1 | AB035356 | Homo sapiens | neurexin 1(NRXN1) |
| NRXN1 | XM_006712137 | Homo sapiens | neurexin 1(NRXN1) |
| NRXN1 | XM_017005319 | Homo sapiens | neurexin 1(NRXN1) |
| NRXN1 | XM_017005317 | Homo sapiens | neurexin 1(NRXN1) |
| NRXN1 | AF064842 | Homo sapiens | neurexin 1(NRXN1) |
| NRXN1 | XM_017005318 | Homo sapiens | neurexin 1(NRXN1) |
| NRXN1 | NM_001330085 | Homo sapiens | neurexin 1(NRXN1) |
| NRXN1 | NM_001330086 | Homo sapiens | neurexin 1(NRXN1) |
| NRXN1 | DC333548 | Homo sapiens | neurexin 1(NRXN1) |
| NRXN1 | NM_001330083 | Homo sapiens | neurexin 1(NRXN1) |
| NRXN1 | NM_001330084 | Homo sapiens | neurexin 1(NRXN1) |
| NRXN1 | NM_001330081 | Homo sapiens | neurexin 1(NRXN1) |
| NRXN1 | NM_001330082 | Homo sapiens | neurexin 1(NRXN1) |
| TYRP1 | DA278582 | Homo sapiens | tyrosinase related protein 1(TYRP1) |
| TYRP1 | CR407683 | Homo sapiens | tyrosinase related protein 1(TYRP1) |
| TYRP1 | X51420 | Homo sapiens | tyrosinase related protein 1(TYRP1) |
| TYRP1 | CD679533 | Homo sapiens | tyrosinase related protein 1(TYRP1) |
| TYRP1 | XR_001746372 | Homo sapiens | tyrosinase related protein 1(TYRP1) |
| TYRP1 | NM_000550 | Homo sapiens | tyrosinase related protein 1(TYRP1) |
| TYRP1 | BC052608 | Homo sapiens | tyrosinase related protein 1(TYRP1) |
| TYRP1 | AL600654 | Homo sapiens | tyrosinase related protein 1(TYRP1) |
| TYRP1 | X51455 | Homo sapiens | tyrosinase related protein 1(TYRP1) |
| TYRP1 | AK297887 | Homo sapiens | tyrosinase related protein 1(TYRP1) |
| NRXN3 | AK310235 | Homo sapiens | neurexin 3(NRXN3) |
| NRXN3 | AK056530 | Homo sapiens | neurexin 3(NRXN3) |
| NRXN3 | NM_001272020 | Homo sapiens | neurexin 3(NRXN3) |
| NRXN3 | XM_017021790 | Homo sapiens | neurexin 3(NRXN3) |
| NRXN3 | BC068469 | Homo sapiens | neurexin 3(NRXN3) |
| NRXN3 | DA243312 | Homo sapiens | neurexin 3(NRXN3) |
| NRXN3 | XM_017021792 | Homo sapiens | neurexin 3(NRXN3) |
| NRXN3 | BC142649 | Homo sapiens | neurexin 3(NRXN3) |
| NRXN3 | XM_017021791 | Homo sapiens | neurexin 3(NRXN3) |
| NRXN3 | XM_017021794 | Homo sapiens | neurexin 3(NRXN3) |
| NRXN3 | XM_017021793 | Homo sapiens | neurexin 3(NRXN3) |
| NRXN3 | XM_017021796 | Homo sapiens | neurexin 3(NRXN3) |
| NRXN3 | XM_017021795 | Homo sapiens | neurexin 3(NRXN3) |
| NRXN3 | XM_017021798 | Homo sapiens | neurexin 3(NRXN3) |
| NRXN3 | XM_017021797 | Homo sapiens | neurexin 3(NRXN3) |
| NRXN3 | XM_011537377 | Homo sapiens | neurexin 3(NRXN3) |
| NRXN3 | XM_017021799 | Homo sapiens | neurexin 3(NRXN3) |
| NRXN3 | NM_001330195 | Homo sapiens | neurexin 3(NRXN3) |
| NRXN3 | XM_006720323 | Homo sapiens | neurexin 3(NRXN3) |
| NRXN3 | AJ493127 | Homo sapiens | neurexin 3(NRXN3) |
| NRXN3 | XM_006720322 | Homo sapiens | neurexin 3(NRXN3) |
| NRXN3 | XR_001750599 | Homo sapiens | neurexin 3(NRXN3) |
| NRXN3 | DA124460 | Homo sapiens | neurexin 3(NRXN3) |
| NRXN3 | XM_005268218 | Homo sapiens | neurexin 3(NRXN3) |
| NRXN3 | XM_011537373 | Homo sapiens | neurexin 3(NRXN3) |
| NRXN3 | NM_001105250 | Homo sapiens | neurexin 3(NRXN3) |
| NRXN3 | XM_011537370 | Homo sapiens | neurexin 3(NRXN3) |
| NRXN3 | XM_011537371 | Homo sapiens | neurexin 3(NRXN3) |
| NRXN3 | XM_011537372 | Homo sapiens | neurexin 3(NRXN3) |
| NRXN3 | XM_011537366 | Homo sapiens | neurexin 3(NRXN3) |
| NRXN3 | XM_017021800 | Homo sapiens | neurexin 3(NRXN3) |
| NRXN3 | XM_011537367 | Homo sapiens | neurexin 3(NRXN3) |
| NRXN3 | AK096247 | Homo sapiens | neurexin 3(NRXN3) |
| NRXN3 | NM_138970 | Homo sapiens | neurexin 3(NRXN3) |
| NRXN3 | NR_073547 | Homo sapiens | neurexin 3(NRXN3) |
| NRXN3 | XM_011537368 | Homo sapiens | neurexin 3(NRXN3) |
| NRXN3 | KM_017021802 | Homo sapiens | neurexin 3(NRXN3) |
| NRXN3 | NR_073546 | Homo sapiens | neurexin 3(NRXN3) |
| NRXN3 | XM_011537369 | Homo sapiens | neurexin 3(NRXN3) |
| NRXN3 | XM_017021801 | Homo sapiens | neurexin 3(NRXN3) |
| NRXN3 | XM_017021804 | Homo sapiens | neurexin 3(NRXN3) |
| NRXN3 | XM_017021803 | Homo sapiens | neurexin 3(NRXN3) |
| NRXN3 | XM_017021806 | Homo sapiens | neurexin 3(NRXN3) |
| NRXN3 | XM_017021805 | Homo sapiens | neurexin 3(NRXN3) |
| NRXN3 | DA083962 | Homo sapiens | neurexin 3(NRXN3) |
| NRXN3 | XM_017021807 | Homo sapiens | neurexin 3(NRXN3) |
| NRXN3 | NM_004796 | Homo sapiens | neurexin 3(NRXN3) |
| NRXN3 | XM_011537363 | Homo sapiens | neurexin 3(NRXN3) |
| NRXN3 | XM_011537364 | Homo sapiens | neurexin 3(NRXN3) |
| NRXN3 | XM_011537365 | Homo sapiens | neurexin 3(NRXN3) |
| NRXN3 | AL833739 | Homo sapiens | neurexin 3(NRXN3) |
| NRXN3 | XR_943563 | Homo sapiens | neurexin 3(NRXN3) |
| NRXN3 | DA213090 | Homo sapiens | neurexin 3(NRXN3) |
| NRXN3 | XR_943562 | Homo sapiens | neurexin 3(NRXN3) |
| NRXN3 | XR_943561 | Homo sapiens | neurexin 3(NRXN3) |
| NRXN3 | XR_001750609 | Homo sapiens | neurexin 3(NRXN3) |
| NRXN3 | XR_001750608 | Homo sapiens | neurexin 3(NRXN3) |
| NRXN3 | XR_001750605 | Homo sapiens | neurexin 3(NRXN3) |
| NRXN3 | XR_001750604 | Homo sapiens | neurexin 3(NRXN3) |
| NRXN3 | XR_001750607 | Homo sapiens | neurexin 3(NRXN3) |
| NRXN3 | XR_001750606 | Homo sapiens | neurexin 3(NRXN3) |
| NRXN3 | XR_001750601 | Homo sapiens | neurexin 3(NRXN3) |
| NRXN3 | XR_001750600 | Homo sapiens | neurexin 3(NRXN3) |
| NRXN3 | XR_001750603 | Homo sapiens | neurexin 3(NRXN3) |
| NRXN3 | BC059368 | Homo sapiens | neurexin 3(NRXN3) |
| NRXN3 | XR_001750602 | Homo sapiens | neurexin 3(NRXN3) |
| NRXN3 | DA128446 | Homo sapiens | neurexin 3(NRXN3) |
| NRXN3 | XR_001750610 | Homo sapiens | neurexin 3(NRXN3) |
| NRXN3 | AB018286 | Homo sapiens | neurexin 3(NRXN3) |
| NRXN3 | BC152457 | Homo sapiens | neurexin 3(NRXN3) |
| NRXN3 | BC150194 | Homo sapiens | neurexin 3(NRXN3) |
| NRXN3 | AJ316284 | Homo sapiens | neurexin 3(NRXN3) |
| NRXN3 | AK126795 | Homo sapiens | neurexin 3(NRXN3) |
| ASCL1 | CF454566 | Homo sapiens | achaete-scute family bHLH transcription factor 1(ASCL1) |
| ASCL1 | BC002341 | Homo sapiens | achaete-scute family bHLH transcription factor 1(ASCL1) |
| ASCL1 | L08424 | Homo sapiens | achaete-scute family bHLH transcription factor 1(ASCL1) |
| ASCL1 | NM_004316 | Homo sapiens | achaete-scute family bHLH transcription factor 1(ASCL1) |
| ASCL1 | BC004425 | Homo sapiens | achaete-scute family bHLH transcription factor 1(ASCL1) |
| ASCL1 | DA005289 | Homo sapiens | achaete-scute family bHLH transcription factor 1(ASCL1) |
| ASCL1 | BC003134 | Homo sapiens | achaete-scute family bHLH transcription factor 1(ASCL1) |
| ASCL1 | AW071836 | Homo sapiens | achaete-scute family bHLH transcription factor 1(ASCL1) |
| ASCL1 | EU176303 | Homo sapiens | achaete-scute family bHLH transcription factor 1(ASCL1) |
| ASCL1 | AK290539 | Homo sapiens | achaete-scute family bHLH transcription factor 1(ASCL1) |
| ASCL1 | BC001638 | Homo sapiens | achaete-scute family bHLH transcription factor 1(ASCL1) |
| ASCL1 | BC031299 | Homo sapiens | achaete-scute family bHLH transcription factor 1(ASCL1) |
| ASCL1 | AK314114 | Homo sapiens | achaete-scute family bHLH transcription factor 1(ASCL1) |
| ASCL1 | DQ894571 | Homo sapiens | achaete-scute family bHLH transcription factor 1(ASCL1) |
| ERBB3 | M34309 | Homo sapiens | erb-b2 receptor tyrosine kinase 3(ERBB3) |
| ERBB3 | AK295650 | Homo sapiens | erb-b2 receptor tyrosine kinase 3(ERBB3) |
| ERBB3 | U88360 | Homo sapiens | erb-b2 receptor tyrosine kinase 3(ERBB3) |
| ERBB3 | BM837872 | Homo sapiens | erb-b2 receptor tyrosine kinase 3(ERBB3) |
| ERBB3 | NM_001982 | Homo sapiens | erb-b2 receptor tyrosine kinase 3(ERBB3) |
| ERBB3 | BC082992 | Homo sapiens | erb-b2 receptor tyrosine kinase 3(ERBB3) |
| ERBB3 | BT007226 | Homo sapiens | erb-b2 receptor tyrosine kinase 3(ERBB3) |
| ERBB3 | NM_001005915 | Homo sapiens | erb-b2 receptor tyrosine kinase 3(ERBB3) |
| ERBB3 | BU674508 | Homo sapiens | erb-b2 receptor tyrosine kinase 3(ERBB3) |
| ERBB3 | U88359 | Homo sapiens | erb-b2 receptor tyrosine kinase 3(ERBB3) |
| ERBB3 | U88358 | Homo sapiens | erb-b2 receptor tyrosine kinase 3(ERBB3) |
| ERBB3 | U88357 | Homo sapiens | erb-b2 receptor tyrosine kinase 3(ERBB3) |
| ERBB3 | M29366 | Homo sapiens | erb-b2 receptor tyrosine kinase 3(ERBB3) |
| ERBB3 | BC041579 | Homo sapiens | erb-b2 receptor tyrosine kinase 3(ERBB3) |
| ERBB3 | AK291681 | Homo sapiens | erb-b2 receptor tyrosine kinase 3(ERBB3) |
| ERBB3 | AU100462 | Homo sapiens | erb-b2 receptor tyrosine kinase 3(ERBB3) |
| ERBB3 | DC347955 | Homo sapiens | erb-b2 receptor tyrosine kinase 3(ERBB3) |
| ERBB3 | AA524528 | Homo sapiens | erb-b2 receptor tyrosine kinase 3(ERBB3) |
| ERBB3 | AK294719 | Homo sapiens | erb-b2 receptor tyrosine kinase 3(ERBB3) |
| ERBB3 | DC344156 | Homo sapiens | erb-b2 receptor tyrosine kinase 3(ERBB3) |
| ERBB3 | BX641868 | Homo sapiens | erb-b2 receptor tyrosine kinase 3(ERBB3) |
| ERBB3 | AK300909 | Homo sapiens | erb-b2 receptor tyrosine kinase 3(ERBB3) |
| ERBB3 | AK125028 | Homo sapiens | erb-b2 receptor tyrosine kinase 3(ERBB3) |
| ERBB3 | AK124710 | Homo sapiens | erb-b2 receptor tyrosine kinase 3(ERBB3) |
| ERBB3 | S61953 | Homo sapiens | erb-b2 receptor tyrosine kinase 3(ERBB3) |
| ERBB3 | BC002706 | Homo sapiens | erb-b2 receptor tyrosine kinase 3(ERBB3) |
| ERBB4 | XM_017003581 | Homo sapiens | erb-b2 receptor tyrosine kinase 4(ERBB4) |
| ERBB4 | AB209697 | Homo sapiens | erb-b2 receptor tyrosine kinase 4(ERBB4) |
| ERBB4 | XM_017003580 | Homo sapiens | erb-b2 receptor tyrosine kinase 4(ERBB4) |
| ERBB4 | AI793060 | Homo sapiens | erb-b2 receptor tyrosine kinase 4(ERBB4) |
| ERBB4 | XM_017003582 | Homo sapiens | erb-b2 receptor tyrosine kinase 4(ERBB4) |
| ERBB4 | AK024204 | Homo sapiens | erb-b2 receptor tyrosine kinase 4(ERBB4) |
| ERBB4 | XM_005246377 | Homo sapiens | erb-b2 receptor tyrosine kinase 4(ERBB4) |
| ERBB4 | XM_005246376 | Homo sapiens | erb-b2 receptor tyrosine kinase 4(ERBB4) |
| ERBB4 | KT281867 | Homo sapiens | erb-b2 receptor tyrosine kinase 4(ERBB4) |
| ERBB4 | NM_005235 | Homo sapiens | erb-b2 receptor tyrosine kinase 4(ERBB4) |
| ERBB4 | XM_006712364 | Homo sapiens | erb-b2 receptor tyrosine kinase 4(ERBB4) |
| ERBB4 | AF007153 | Homo sapiens | erb-b2 receptor tyrosine kinase 4(ERBB4) |
| ERBB4 | BC143741 | Homo sapiens | erb-b2 receptor tyrosine kinase 4(ERBB4) |
| ERBB4 | CR627023 | Homo sapiens | erb-b2 receptor tyrosine kinase 4(ERBB4) |
| ERBB4 | L07868 | Homo sapiens | erb-b2 receptor tyrosine kinase 4(ERBB4) |
| ERBB4 | BC143747 | Homo sapiens | erb-b2 receptor tyrosine kinase 4(ERBB4) |
| ERBB4 | XM_017003578 | Homo sapiens | erb-b2 receptor tyrosine kinase 4(ERBB4) |
| ERBB4 | XM_017003577 | Homo sapiens | erb-b2 receptor tyrosine kinase 4(ERBB4) |
| ERBB4 | BC143749 | Homo sapiens | erb-b2 receptor tyrosine kinase 4(ERBB4) |
| ERBB4 | BQ015804 | Homo sapiens | erb-b2 receptor tyrosine kinase 4(ERBB4) |
| ERBB4 | AK308606 | Homo sapiens | erb-b2 receptor tyrosine kinase 4(ERBB4) |
| ERBB4 | AK126298 | Homo sapiens | erb-b2 receptor tyrosine kinase 4(ERBB4) |
| ERBB4 | KT310076 | Homo sapiens | erb-b2 receptor tyrosine kinase 4(ERBB4) |
| ERBB4 | XM_017003579 | Homo sapiens | erb-b2 receptor tyrosine kinase 4(ERBB4) |
| ERBB4 | BC112199 | Homo sapiens | erb-b2 receptor tyrosine kinase 4(ERBB4) |
| ERBB4 | NM_001042599 | Homo sapiens | erb-b2 receptor tyrosine kinase 4(ERBB4) |
| EPCAM | BC014785 | Homo sapiens | epithelial cell adhesion molecule(EPCAM) |
| EPCAM | CR542259 | Homo sapiens | epithelial cell adhesion molecule(EPCAM) |
| EPCAM | DA963872 | Homo sapiens | epithelial cell adhesion molecule(EPCAM) |
| EPCAM | M32325 | Homo sapiens | epithelial cell adhesion molecule(EPCAM) |
| EPCAM | M26481 | Homo sapiens | epithelial cell adhesion molecule(EPCAM) |
| EPCAM | M32306 | Homo sapiens | epithelial cell adhesion molecule(EPCAM) |
| EPCAM | DQ891338 | Homo sapiens | epithelial cell adhesion molecule(EPCAM) |
| EPCAM | M33011 | Homo sapiens | epithelial cell adhesion molecule(EPCAM) |
| EPCAM | NM_002354 | Homo sapiens | epithelial cell adhesion molecule(EPCAM) |
| EPCAM | AK026585 | Homo sapiens | epithelial cell adhesion molecule(EPCAM) |
| EPCAM | X14758 | Homo sapiens | epithelial cell adhesion molecule(EPCAM) |
| EPCAM | BP262680 | Homo sapiens | epithelial cell adhesion molecule(EPCAM) |
| EPCAM | AW050533 | Homo sapiens | epithelial cell adhesion molecule(EPCAM) |
| EPCAM | CR542283 | Homo sapiens | epithelial cell adhesion molecule(EPCAM) |
| EPCAM | DQ894520 | Homo sapiens | epithelial cell adhesion molecule(EPCAM) |
| EPCAM | DB044092 | Homo sapiens | epithelial cell adhesion molecule(EPCAM) |
| NCAM1 | AK054570 | Homo sapiens | neural cell adhesion molecule 1(NCAM1) |
| NCAM1 | AK056258 | Homo sapiens | neural cell adhesion molecule 1(NCAM1) |
| NCAM1 | BC014205 | Homo sapiens | neural cell adhesion molecule 1(NCAM1) |
| NCAM1 | U63041 | Homo sapiens | neural cell adhesion molecule 1(NCAM1) |
| NCAM1 | X59400 | Homo sapiens | neural cell adhesion molecule 1(NCAM1) |
| NCAM1 | GQ129418 | Homo sapiens | neural cell adhesion molecule 1(NCAM1) |
| NCAM1 | NM_000615 | Homo sapiens | neural cell adhesion molecule 1(NCAM1) |
| NCAM1 | BC047244 | Homo sapiens | neural cell adhesion molecule 1(NCAM1) |
| NCAM1 | BC029119 | Homo sapiens | neural cell adhesion molecule 1(NCAM1) |
| NCAM1 | X55322 | Homo sapiens | neural cell adhesion molecule 1(NCAM1) |
| NCAM1 | AK057509 | Homo sapiens | neural cell adhesion molecule 1(NCAM1) |
| NCAM1 | AW131780 | Homo sapiens | neural cell adhesion molecule 1(NCAM1) |
| NCAM1 | M17409 | Homo sapiens | neural cell adhesion molecule 1(NCAM1) |
| NCAM1 | AL832563 | Homo sapiens | neural cell adhesion molecule 1(NCAM1) |
| NCAM1 | AF005070 | Homo sapiens | neural cell adhesion molecule 1(NCAM1) |
| NCAM1 | AB209443 | Homo sapiens | neural cell adhesion molecule 1(NCAM1) |
| NCAM1 | NM_001076682 | Homo sapiens | neural cell adhesion molecule 1(NCAM1) |
| NCAM1 | S73101 | Homo sapiens | neural cell adhesion molecule 1(NCAM1) |
| NCAM1 | DA231540 | Homo sapiens | neural cell adhesion molecule 1(NCAM1) |
| NCAM1 | M22094 | Homo sapiens | neural cell adhesion molecule 1(NCAM1) |
| NCAM1 | AK303232 | Homo sapiens | neural cell adhesion molecule 1(NCAM1) |
| NCAM1 | BC019845 | Homo sapiens | neural cell adhesion molecule 1(NCAM1) |
| NCAM1 | AA029449 | Homo sapiens | neural cell adhesion molecule 1(NCAM1) |
| NCAM1 | NM_181351 | Homo sapiens | neural cell adhesion molecule 1(NCAM1) |
| NCAM1 | S71824 | Homo sapiens | neural cell adhesion molecule 1(NCAM1) |
| NCAM1 | EU832805 | Homo sapiens | neural cell adhesion molecule 1(NCAM1) |
| NCAM1 | NM_001242607 | Homo sapiens | neural cell adhesion molecule 1(NCAM1) |
| NCAM1 | X16841 | Homo sapiens | neural cell adhesion molecule 1(NCAM1) |
| NCAM1 | NM_001242608 | Homo sapiens | neural cell adhesion molecule 1(NCAM1) |
| NCAM1 | AK054929 | Homo sapiens | neural cell adhesion molecule 1(NCAM1) |
| NCAM1 | M17410 | Homo sapiens | neural cell adhesion molecule 1(NCAM1) |
| NCAM1 | DA337649 | Homo sapiens | neural cell adhesion molecule 1(NCAM1) |
| NCAM1 | AK314589 | Homo sapiens | neural cell adhesion molecule 1(NCAM1) |
| NCAM1 | AK292453 | Homo sapiens | neural cell adhesion molecule 1(NCAM1) |
| NCAM1 | AK299865 | Homo sapiens | neural cell adhesion molecule 1(NCAM1) |
| SLIT2 | AB017168 | Homo sapiens | slit guidance ligand 2(SLIT2) |
| SLIT2 | XM_017008845 | Homo sapiens | slit guidance ligand 2(SLIT2) |
| SLIT2 | AK308444 | Homo sapiens | slit guidance ligand 2(SLIT2) |
| SLIT2 | BI494498 | Homo sapiens | slit guidance ligand 2(SLIT2) |
| SLIT2 | XM_011513909 | Homo sapiens | slit guidance ligand 2(SLIT2) |
| SLIT2 | BC117190 | Homo sapiens | slit guidance ligand 2(SLIT2) |
| SLIT2 | BC143978 | Homo sapiens | slit guidance ligand 2(SLIT2) |
| SLIT2 | AF055585 | Homo sapiens | slit guidance ligand 2(SLIT2) |
| SLIT2 | BF112143 | Homo sapiens | slit guidance ligand 2(SLIT2) |
| SLIT2 | NM_004787 | Homo sapiens | slit guidance ligand 2(SLIT2) |
| SLIT2 | AK027326 | Homo sapiens | slit guidance ligand 2(SLIT2) |
| SLIT2 | DB207352 | Homo sapiens | slit guidance ligand 2(SLIT2) |
| SLIT2 | DA721496 | Homo sapiens | slit guidance ligand 2(SLIT2) |
| SLIT2 | BM474839 | Homo sapiens | slit guidance ligand 2(SLIT2) |
| SLIT2 | AA489463 | Homo sapiens | slit guidance ligand 2(SLIT2) |
| SLIT2 | XM_011513910 | Homo sapiens | slit guidance ligand 2(SLIT2) |
| SLIT2 | XM_006713986 | Homo sapiens | slit guidance ligand 2(SLIT2) |
| SLIT2 | CV370469 | Homo sapiens | slit guidance ligand 2(SLIT2) |
| SLIT2 | NM_001289136 | Homo sapiens | slit guidance ligand 2(SLIT2) |
| SLIT2 | AK126459 | Homo sapiens | slit guidance ligand 2(SLIT2) |
| SLIT2 | NM_001289135 | Homo sapiens | slit guidance ligand 2(SLIT2) |
| SLIT2 | AF133270 | Homo sapiens | slit guidance ligand 2(SLIT2) |
| SLIT2 | XM_005248211 | Homo sapiens | slit guidance ligand 2(SLIT2) |
| MUC6 | AY458429 | Homo sapiens | mucin 6, oligomeric mucus/gel-forming(MUC6) |
| MUC6 | AK092533 | Homo sapiens | mucin 6, oligomeric mucus/gel-forming(MUC6) |
| MUC6 | NM_005961 | Homo sapiens | mucin 6, oligomeric mucus/gel-forming(MUC6) |
| MUC6 | AK098503 | Homo sapiens | mucin 6, oligomeric mucus/gel-forming(MUC6) |
| MUC6 | AY312160 | Homo sapiens | mucin 6, oligomeric mucus/gel-forming(MUC6) |
| MUC6 | U97698 | Homo sapiens | mucin 6, oligomeric mucus/gel-forming(MUC6) |
| MUC6 | AK096772 | Homo sapiens | mucin 6, oligomeric mucus/gel-forming(MUC6) |
| TBX1 | AF012131 | Homo sapiens | T-box 1(TBX1) |
| TBX1 | XM_017028927 | Homo sapiens | T-box 1(TBX1) |
| TBX1 | AF012130 | Homo sapiens | T-box 1(TBX1) |
| TBX1 | XM_017028928 | Homo sapiens | T-box 1(TBX1) |
| TBX1 | XM_017028925 | Homo sapiens | T-box 1(TBX1) |
| TBX1 | NM_005992 | Homo sapiens | T-box 1(TBX1) |
| TBX1 | XM_017028926 | Homo sapiens | T-box 1(TBX1) |
| TBX1 | NM_080646 | Homo sapiens | T-box 1(TBX1) |
| TBX1 | AF373867 | Homo sapiens | T-box 1(TBX1) |
| TBX1 | NM_080647 | Homo sapiens | T-box 1(TBX1) |
| TBX1 | GU014843 | Homo sapiens | T-box 1(TBX1) |
| TBX1 | XM_006724312 | Homo sapiens | T-box 1(TBX1) |
| NTRK2 | AL713745 | Homo sapiens | neurotrophic receptor tyrosine kinase 2(NTRK2) |
| NTRK2 | XM_005252003 | Homo sapiens | neurotrophic receptor tyrosine kinase 2(NTRK2) |
| NTRK2 | AF410901 | Homo sapiens | neurotrophic receptor tyrosine kinase 2(NTRK2) |
| NTRK2 | XM_005252001 | Homo sapiens | neurotrophic receptor tyrosine kinase 2(NTRK2) |
| NTRK2 | AF410900 | Homo sapiens | neurotrophic receptor tyrosine kinase 2(NTRK2) |
| NTRK2 | NM_001291937 | Homo sapiens | neurotrophic receptor tyrosine kinase 2(NTRK2) |
| NTRK2 | XM_005252007 | Homo sapiens | neurotrophic receptor tyrosine kinase 2(NTRK2) |
| NTRK2 | XM_005252006 | Homo sapiens | neurotrophic receptor tyrosine kinase 2(NTRK2) |
| NTRK2 | XM_005252004 | Homo sapiens | neurotrophic receptor tyrosine kinase 2(NTRK2) |
| NTRK2 | HY311899 | Homo sapiens | neurotrophic receptor tyrosine kinase 2(NTRK2) |
| NTRK2 | AL833196 | Homo sapiens | neurotrophic receptor tyrosine kinase 2(NTRK2) |
| NTRK2 | S76473 | Homo sapiens | neurotrophic receptor tyrosine kinase 2(NTRK2) |
| NTRK2 | S76474 | Homo sapiens | neurotrophic receptor tyrosine kinase 2(NTRK2) |
| NTRK2 | U12140 | Homo sapiens | neurotrophic receptor tyrosine kinase 2(NTRK2) |
| NTRK2 | BX649001 | Homo sapiens | neurotrophic receptor tyrosine kinase 2(NTRK2) |
| NTRK2 | AK123824 | Homo sapiens | neurotrophic receptor tyrosine kinase 2(NTRK2) |
| NTRK2 | AF410898 | Homo sapiens | neurotrophic receptor tyrosine kinase 2(NTRK2) |
| NTRK2 | AF410899 | Homo sapiens | neurotrophic receptor tyrosine kinase 2(NTRK2) |
| NTRK2 | AF508964 | Homo sapiens | neurotrophic receptor tyrosine kinase 2(NTRK2) |
| NTRK2 | BC031309 | Homo sapiens | neurotrophic receptor tyrosine kinase 2(NTRK2) |
| NTRK2 | AK289904 | Homo sapiens | neurotrophic receptor tyrosine kinase 2(NTRK2) |
| NTRK2 | BC075804 | Homo sapiens | neurotrophic receptor tyrosine kinase 2(NTRK2) |
| NTRK2 | AL533181 | Homo sapiens | neurotrophic receptor tyrosine kinase 2(NTRK2) |
| NTRK2 | XM_017014760 | Homo sapiens | neurotrophic receptor tyrosine kinase 2(NTRK2) |
| NTRK2 | KM_017014761 | Homo sapiens | neurotrophic receptor tyrosine kinase 2(NTRK2) |
| NTRK2 | XM_017014759 | Homo sapiens | neurotrophic receptor tyrosine kinase 2(NTRK2) |
| NTRK2 | AK294285 | Homo sapiens | neurotrophic receptor tyrosine kinase 2(NTRK2) |
| NTRK2 | AJ420458 | Homo sapiens | neurotrophic receptor tyrosine kinase 2(NTRK2) |
| NTRK2 | XM_017014755 | Homo sapiens | neurotrophic receptor tyrosine kinase 2(NTRK2) |
| NTRK2 | DA283111 | Homo sapiens | neurotrophic receptor tyrosine kinase 2(NTRK2) |
| NTRK2 | XM_017014756 | Homo sapiens | neurotrophic receptor tyrosine kinase 2(NTRK2) |
| NTRK2 | XM_017014757 | Homo sapiens | neurotrophic receptor tyrosine kinase 2(NTRK2) |
| NTRK2 | AB209118 | Homo sapiens | neurotrophic receptor tyrosine kinase 2(NTRK2) |
| NTRK2 | XM_011518720 | Homo sapiens | neurotrophic receptor tyrosine kinase 2(NTRK2) |
| NTRK2 | XM_017014758 | Homo sapiens | neurotrophic receptor tyrosine kinase 2(NTRK2) |
| NTRK2 | BE466753 | Homo sapiens | neurotrophic receptor tyrosine kinase 2(NTRK2) |
| NTRK2 | BC031835 | Homo sapiens | neurotrophic receptor tyrosine kinase 2(NTRK2) |
| NTRK2 | XM_011518718 | Homo sapiens | neurotrophic receptor tyrosine kinase 2(NTRK2) |
| NTRK2 | BE148667 | Homo sapiens | neurotrophic receptor tyrosine kinase 2(NTRK2) |
| NTRK2 | NM_001018066 | Homo sapiens | neurotrophic receptor tyrosine kinase 2(NTRK2) |
| NTRK2 | NM_001018065 | Homo sapiens | neurotrophic receptor tyrosine kinase 2(NTRK2) |
| NTRK2 | NM_001018064 | Homo sapiens | neurotrophic receptor tyrosine kinase 2(NTRK2) |
| NTRK2 | NM_001007097 | Homo sapiens | neurotrophic receptor tyrosine kinase 2(NTRK2) |
| NTRK2 | XM_017014751 | Homo sapiens | neurotrophic receptor tyrosine kinase 2(NTRK2) |
| NTRK2 | NM_006180 | Homo sapiens | neurotrophic receptor tyrosine kinase 2(NTRK2) |
| NTRK2 | XM_017014752 | Homo sapiens | neurotrophic receptor tyrosine kinase 2(NTRK2) |
| NTRK2 | XM_017014753 | Homo sapiens | neurotrophic receptor tyrosine kinase 2(NTRK2) |
| NTRK2 | XM_017014754 | Homo sapiens | neurotrophic receptor tyrosine kinase 2(NTRK2) |
| NTRK2 | AF086101 | Homo sapiens | neurotrophic receptor tyrosine kinase 2(NTRK2) |
| NTRK2 | DA449367 | Homo sapiens | neurotrophic receptor tyrosine kinase 2(NTRK2) |
| NTRK2 | X75958 | Homo sapiens | neurotrophic receptor tyrosine kinase 2(NTRK2) |
| NTRK2 | AF400441 | Homo sapiens | neurotrophic receptor tyrosine kinase 2(NTRK2) |
| NTRK2 | AK092267 | Homo sapiens | neurotrophic receptor tyrosine kinase 2(NTRK2) |
| NTRK2 | KC855566 | Homo sapiens | neurotrophic receptor tyrosine kinase 2(NTRK2) |
| NTRK2 | AW235842 | Homo sapiens | neurotrophic receptor tyrosine kinase 2(NTRK2) |
| NOP58 | BC018805 | Homo sapiens | NOP58 ribonucleoprotein(NOP58) |
| NOP58 | AK307396 | Homo sapiens | NOP58 ribonucleoprotein(NOP58) |
| NOP58 | BP245255 | Homo sapiens | NOP58 ribonucleoprotein(NOP58) |
| NOP58 | AL117554 | Homo sapiens | NOP58 ribonucleoprotein(NOP58) |
| NOP58 | DQ890868 | Homo sapiens | NOP58 ribonucleoprotein(NOP58) |
| NOP58 | EU176490 | Homo sapiens | NOP58 ribonucleoprotein(NOP58) |
| NOP58 | AK300844 | Homo sapiens | NOP58 ribonucleoprotein(NOP58) |
| NOP58 | AK023975 | Homo sapiens | NOP58 ribonucleoprotein(NOP58) |
| NOP58 | AF263608 | Homo sapiens | NOP58 ribonucleoprotein(NOP58) |
| NOP58 | BC009306 | Homo sapiens | NOP58 ribonucleoprotein(NOP58) |
| NOP58 | DQ586402 | Homo sapiens | NOP58 ribonucleoprotein(NOP58) |
| NOP58 | BC001707 | Homo sapiens | NOP58 ribonucleoprotein(NOP58) |
| NOP58 | BC032592 | Homo sapiens | NOP58 ribonucleoprotein(NOP58) |
| NOP58 | AF161469 | Homo sapiens | NOP58 ribonucleoprotein(NOP58) |
| NOP58 | NM_015934 | Homo sapiens | NOP58 ribonucleoprotein(NOP58) |
| NOP58 | AF123534 | Homo sapiens | NOP58 ribonucleoprotein(NOP58) |
| CDKN2C | BE783451 | Homo sapiens | cyclin dependent kinase inhibitor 2C(CDKN2C) |
| CDKN2C | DQ891516 | Homo sapiens | cyclin dependent kinase inhibitor 2C(CDKN2C) |
| CDKN2C | NM_078626 | Homo sapiens | cyclin dependent kinase inhibitor 2C(CDKN2C) |
| CDKN2C | U17074 | Homo sapiens | cyclin dependent kinase inhibitor 2C(CDKN2C) |
| CDKN2C | DQ894707 | Homo sapiens | cyclin dependent kinase inhibitor 2C(CDKN2C) |
| CDKN2C | AK021794 | Homo sapiens | cyclin dependent kinase inhibitor 2C(CDKN2C) |
| CDKN2C | BC005041 | Homo sapiens | cyclin dependent kinase inhibitor 2C(CDKN2C) |
| CDKN2C | BE831176 | Homo sapiens | cyclin dependent kinase inhibitor 2C(CDKN2C) |
| CDKN2C | AK091170 | Homo sapiens | cyclin dependent kinase inhibitor 2C(CDKN2C) |
| CDKN2C | AF041248 | Homo sapiens | cyclin dependent kinase inhibitor 2C(CDKN2C) |
| CDKN2C | NM_001262 | Homo sapiens | cyclin dependent kinase inhibitor 2C(CDKN2C) |
| CDKN2C | BC000598 | Homo sapiens | cyclin dependent kinase inhibitor 2C(CDKN2C) |
| CDKN2C | CR450289 | Homo sapiens | cyclin dependent kinase inhibitor 2C(CDKN2C) |
| CDKN2C | BC016173 | Homo sapiens | cyclin dependent kinase inhibitor 2C(CDKN2C) |
| CDKN2C | BC017036 | Homo sapiens | cyclin dependent kinase inhibitor 2C(CDKN2C) |
| PRRX1 | AA452918 | Homo sapiens | paired related homeobox 1(PRRX1) |
| PRRX1 | BC074993 | Homo sapiens | paired related homeobox 1(PRRX1) |
| PRRX1 | EU446648 | Homo sapiens | paired related homeobox 1(PRRX1) |
| PRRX1 | AA758879 | Homo sapiens | paired related homeobox 1(PRRX1) |
| PRRX1 | BI039001 | Homo sapiens | paired related homeobox 1(PRRX1) |
| PRRX1 | XM_006711388 | Homo sapiens | paired related homeobox 1(PRRX1) |
| PRRX1 | AB451463 | Homo sapiens | paired related homeobox 1(PRRX1) |
| PRRX1 | AV750422 | Homo sapiens | paired related homeobox 1(PRRX1) |
| PRRX1 | NM_022716 | Homo sapiens | paired related homeobox 1(PRRX1) |
| PRRX1 | AK225968 | Homo sapiens | paired related homeobox 1(PRRX1) |
| PRRX1 | NM_006902 | Homo sapiens | paired related homeobox 1(PRRX1) |
| USP9X | BC063645 | Homo sapiens | ubiquitin specific peptidase 9, X-linked(USP9X) |
| USP9X | BE925861 | Homo sapiens | ubiquitin specific peptidase 9, X-linked(USP9X) |
| USP9X | DA854185 | Homo sapiens | ubiquitin specific peptidase 9, X-linked(USP9X) |
| USP9X | AB209666 | Homo sapiens | ubiquitin specific peptidase 9, X-linked(USP9X) |
| USP9X | AJ012078 | Homo sapiens | ubiquitin specific peptidase 9, X-linked(USP9X) |
| USP9X | CD619095 | Homo sapiens | ubiquitin specific peptidase 9, X-linked(USP9X) |
| USP9X | BX956755 | Homo sapiens | ubiquitin specific peptidase 9, X-linked(USP9X) |
| USP9X | AA261797 | Homo sapiens | ubiquitin specific peptidase 9, X-linked(USP9X) |
| USP9X | BC046205 | Homo sapiens | ubiquitin specific peptidase 9, X-linked(USP9X) |
| USP9X | AW183065 | Homo sapiens | ubiquitin specific peptidase 9, X-linked(USP9X) |
| USP9X | XM_005272676 | Homo sapiens | ubiquitin specific peptidase 9, X-linked(USP9X) |
| USP9X | XM_005272675 | Homo sapiens | ubiquitin specific peptidase 9, X-linked(USP9X) |
| USP9X | NM_001039591 | Homo sapiens | ubiquitin specific peptidase 9, X-linked(USP9X) |
| USP9X | NM_021906 | Homo sapiens | ubiquitin specific peptidase 9, X-linked(USP9X) |
| USP9X | AF070645 | Homo sapiens | ubiquitin specific peptidase 9, X-linked(USP9X) |
| USP9X | AK294828 | Homo sapiens | ubiquitin specific peptidase 9, X-linked(USP9X) |
| USP9X | NM_004652 | Homo sapiens | ubiquitin specific peptidase 9, X-linked(USP9X) |
| USP9X | HG512500 | Homo sapiens | ubiquitin specific peptidase 9, X-linked(USP9X) |
| USP9X | BC172429 | Homo sapiens | ubiquitin specific peptidase 9, X-linked(USP9X) |
| USP9X | BC014792 | Homo sapiens | ubiquitin specific peptidase 9, X-linked(USP9X) |
| USP9X | NM_001039590 | Homo sapiens | ubiquitin specific peptidase 9, X-linked(USP9X) |
| USP9X | BC054861 | Homo sapiens | ubiquitin specific peptidase 9, X-linked(USP9X) |
| USP9X | X98296 | Homo sapiens | ubiquitin specific peptidase 9, X-linked(USP9X) |
| NTRK3 | NM_001243101 | Homo sapiens | neurotrophic receptor tyrosine kinase 3(NTRK3) |
| NTRK3 | XM_017022241 | Homo sapiens | neurotrophic receptor tyrosine kinase 3(NTRK3) |
| NTRK3 | XM_017022240 | Homo sapiens | neurotrophic receptor tyrosine kinase 3(NTRK3) |
| NTRK3 | XM_017022243 | Homo sapiens | neurotrophic receptor tyrosine kinase 3(NTRK3) |
| NTRK3 | XM_017022242 | Homo sapiens | neurotrophic receptor tyrosine kinase 3(NTRK3) |
| NTRK3 | XM_011521638 | Homo sapiens | neurotrophic receptor tyrosine kinase 3(NTRK3) |
| NTRK3 | XM_017022245 | Homo sapiens | neurotrophic receptor tyrosine kinase 3(NTRK3) |
| NTRK3 | XM_011521637 | Homo sapiens | neurotrophic receptor tyrosine kinase 3(NTRK3) |
| NTRK3 | XM_017022244 | Homo sapiens | neurotrophic receptor tyrosine kinase 3(NTRK3) |
| NTRK3 | AY065844 | Homo sapiens | neurotrophic receptor tyrosine kinase 3(NTRK3) |
| NTRK3 | XM_017022247 | Homo sapiens | neurotrophic receptor tyrosine kinase 3(NTRK3) |
| NTRK3 | XM_017022246 | Homo sapiens | neurotrophic receptor tyrosine kinase 3(NTRK3) |
| NTRK3 | XM_017022249 | Homo sapiens | neurotrophic receptor tyrosine kinase 3(NTRK3) |
| NTRK3 | CD370531 | Homo sapiens | neurotrophic receptor tyrosine kinase 3(NTRK3) |
| NTRK3 | XM_017022248 | Homo sapiens | neurotrophic receptor tyrosine kinase 3(NTRK3) |
| NTRK3 | NM_001007156 | Homo sapiens | neurotrophic receptor tyrosine kinase 3(NTRK3) |
| NTRK3 | CK903553 | Homo sapiens | neurotrophic receptor tyrosine kinase 3(NTRK3) |
| NTRK3 | S76475 | Homo sapiens | neurotrophic receptor tyrosine kinase 3(NTRK3) |
| NTRK3 | S76476 | Homo sapiens | neurotrophic receptor tyrosine kinase 3(NTRK3) |
| NTRK3 | DQ323597 | Homo sapiens | neurotrophic receptor tyrosine kinase 3(NTRK3) |
| NTRK3 | NM_001012338 | Homo sapiens | neurotrophic receptor tyrosine kinase 3(NTRK3) |
| NTRK3 | AF058389 | Homo sapiens | neurotrophic receptor tyrosine kinase 3(NTRK3) |
| NTRK3 | XM_017022250 | Homo sapiens | neurotrophic receptor tyrosine kinase 3(NTRK3) |
| NTRK3 | HY003065 | Homo sapiens | neurotrophic receptor tyrosine kinase 3(NTRK3) |
| NTRK3 | AK297160 | Homo sapiens | neurotrophic receptor tyrosine kinase 3(NTRK3) |
| NTRK3 | XM_017022252 | Homo sapiens | neurotrophic receptor tyrosine kinase 3(NTRK3) |
| NTRK3 | XM_017022251 | Homo sapiens | neurotrophic receptor tyrosine kinase 3(NTRK3) |
| NTRK3 | XM_017022254 | Homo sapiens | neurotrophic receptor tyrosine kinase 3(NTRK3) |
| NTRK3 | XM_017022253 | Homo sapiens | neurotrophic receptor tyrosine kinase 3(NTRK3) |
| NTRK3 | AL134171 | Homo sapiens | neurotrophic receptor tyrosine kinase 3(NTRK3) |
| NTRK3 | BG741347 | Homo sapiens | neurotrophic receptor tyrosine kinase 3(NTRK3) |
| NTRK3 | AF058390 | Homo sapiens | neurotrophic receptor tyrosine kinase 3(NTRK3) |
| NTRK3 | BT007291 | Homo sapiens | neurotrophic receptor tyrosine kinase 3(NTRK3) |
| NTRK3 | BC013693 | Homo sapiens | neurotrophic receptor tyrosine kinase 3(NTRK3) |
| NTRK3 | U05012 | Homo sapiens | neurotrophic receptor tyrosine kinase 3(NTRK3) |
| NTRK3 | AI613045 | Homo sapiens | neurotrophic receptor tyrosine kinase 3(NTRK3) |
| NTRK3 | BI962878 | Homo sapiens | neurotrophic receptor tyrosine kinase 3(NTRK3) |
| NTRK3 | NM_002530 | Homo sapiens | neurotrophic receptor tyrosine kinase 3(NTRK3) |
| NTRK3 | AL109700 | Homo sapiens | neurotrophic receptor tyrosine kinase 3(NTRK3) |
| NTRK3 | XR_001751292 | Homo sapiens | neurotrophic receptor tyrosine kinase 3(NTRK3) |
| NTRK3 | XR_001751293 | Homo sapiens | neurotrophic receptor tyrosine kinase 3(NTRK3) |
| NTRK3 | XM_006720550 | Homo sapiens | neurotrophic receptor tyrosine kinase 3(NTRK3) |
| NTRK3 | KJ534906 | Homo sapiens | neurotrophic receptor tyrosine kinase 3(NTRK3) |
| NTRK3 | KJ534905 | Homo sapiens | neurotrophic receptor tyrosine kinase 3(NTRK3) |
| NTRK3 | NM_001320134 | Homo sapiens | neurotrophic receptor tyrosine kinase 3(NTRK3) |
| NTRK3 | KJ534904 | Homo sapiens | neurotrophic receptor tyrosine kinase 3(NTRK3) |
| NTRK3 | NM_001320135 | Homo sapiens | neurotrophic receptor tyrosine kinase 3(NTRK3) |
| NTRK3 | AK313646 | Homo sapiens | neurotrophic receptor tyrosine kinase 3(NTRK3) |
| NTRK3 | XM_006720543 | Homo sapiens | neurotrophic receptor tyrosine kinase 3(NTRK3) |
| NTRK3 | AF125808 | Homo sapiens | neurotrophic receptor tyrosine kinase 3(NTRK3) |
| NTRK3 | AK309695 | Homo sapiens | neurotrophic receptor tyrosine kinase 3(NTRK3) |
| NTRK3 | BC128249 | Homo sapiens | neurotrophic receptor tyrosine kinase 3(NTRK3) |
| NTRK3 | XM_006720545 | Homo sapiens | neurotrophic receptor tyrosine kinase 3(NTRK3) |
| NTRK3 | XM_006720544 | Homo sapiens | neurotrophic receptor tyrosine kinase 3(NTRK3) |
| NTRK3 | KJ535052 | Homo sapiens | neurotrophic receptor tyrosine kinase 3(NTRK3) |
| NTRK3 | BQ893817 | Homo sapiens | neurotrophic receptor tyrosine kinase 3(NTRK3) |
| NTRK3 | AK302504 | Homo sapiens | neurotrophic receptor tyrosine kinase 3(NTRK3) |
| NTRK3 | AF052184 | Homo sapiens | neurotrophic receptor tyrosine kinase 3(NTRK3) |
| NTRK3 | BM887782 | Homo sapiens | neurotrophic receptor tyrosine kinase 3(NTRK3) |
| NTRK3 | DA789502 | Homo sapiens | neurotrophic receptor tyrosine kinase 3(NTRK3) |
| NTRK3 | BM675026 | Homo sapiens | neurotrophic receptor tyrosine kinase 3(NTRK3) |
| NTRK3 | CB053605 | Homo sapiens | neurotrophic receptor tyrosine kinase 3(NTRK3) |
| NTRK3 | XM_006720549 | Homo sapiens | neurotrophic receptor tyrosine kinase 3(NTRK3) |
| NTRK3 | AK094929 | Homo sapiens | neurotrophic receptor tyrosine kinase 3(NTRK3) |
| NTRK3 | XM_006720548 | Homo sapiens | neurotrophic receptor tyrosine kinase 3(NTRK3) |
| VEGFC | CR541897 | Homo sapiens | vascular endothelial growth factor C(VEGFC) |
| VEGFC | X94216 | Homo sapiens | vascular endothelial growth factor C(VEGFC) |
| VEGFC | DA044965 | Homo sapiens | vascular endothelial growth factor C(VEGFC) |
| VEGFC | U58111 | Homo sapiens | vascular endothelial growth factor C(VEGFC) |
| VEGFC | NM_005429 | Homo sapiens | vascular endothelial growth factor C(VEGFC) |
| VEGFC | BF528469 | Homo sapiens | vascular endothelial growth factor C(VEGFC) |
| VEGFC | BC063685 | Homo sapiens | vascular endothelial growth factor C(VEGFC) |
| VEGFC | AW294939 | Homo sapiens | vascular endothelial growth factor C(VEGFC) |
| VEGFC | DQ894481 | Homo sapiens | vascular endothelial growth factor C(VEGFC) |
| VEGFC | DQ891162 | Homo sapiens | vascular endothelial growth factor C(VEGFC) |
| VEGFC | DQ896666 | Homo sapiens | vascular endothelial growth factor C(VEGFC) |
| VEGFC | U43142 | Homo sapiens | vascular endothelial growth factor C(VEGFC) |
| VEGFC | DQ893345 | Homo sapiens | vascular endothelial growth factor C(VEGFC) |
| VEGFC | BC035212 | Homo sapiens | vascular endothelial growth factor C(VEGFC) |
| VEGFC | AK313879 | Homo sapiens | vascular endothelial growth factor C(VEGFC) |
| KCNJ16 | DA636394 | Homo sapiens | potassium voltage-gated channel subfamily J member 16(KCNJ16) |
| KCNJ16 | XM_006721885 | Homo sapiens | potassium voltage-gated channel subfamily J member 16(KCNJ16) |
| KCNJ16 | NM_001270422 | Homo sapiens | potassium voltage-gated channel subfamily J member 16(KCNJ16) |
| KCNJ16 | XM_005257337 | Homo sapiens | potassium voltage-gated channel subfamily J member 16(KCNJ16) |
| KCNJ16 | AK225944 | Homo sapiens | potassium voltage-gated channel subfamily J member 16(KCNJ16) |
| KCNJ16 | XM_006721887 | Homo sapiens | potassium voltage-gated channel subfamily J member 16(KCNJ16) |
| KCNJ16 | XM_006721886 | Homo sapiens | potassium voltage-gated channel subfamily J member 16(KCNJ16) |
| KCNJ16 | NM_001291623 | Homo sapiens | potassium voltage-gated channel subfamily J member 16(KCNJ16) |
| KCNJ16 | NM_001291624 | Homo sapiens | potassium voltage-gated channel subfamily J member 16(KCNJ16) |
| KCNJ16 | NM_001291622 | Homo sapiens | potassium voltage-gated channel subfamily J member 16(KCNJ16) |
| KCNJ16 | AF153815 | Homo sapiens | potassium voltage-gated channel subfamily J member 16(KCNJ16) |
| KCNJ16 | AF153816 | Homo sapiens | potassium voltage-gated channel subfamily J member 16(KCNJ16) |
| KCNJ16 | AF153817 | Homo sapiens | potassium voltage-gated channel subfamily J member 16(KCNJ16) |
| KCNJ16 | DA071381 | Homo sapiens | potassium voltage-gated channel subfamily J member 16(KCNJ16) |
| KCNJ16 | NM_170742 | Homo sapiens | potassium voltage-gated channel subfamily J member 16(KCNJ16) |
| KCNJ16 | BC030961 | Homo sapiens | potassium voltage-gated channel subfamily J member 16(KCNJ16) |
| KCNJ16 | NM_170741 | Homo sapiens | potassium voltage-gated channel subfamily J member 16(KCNJ16) |
| KCNJ16 | BC033038 | Homo sapiens | potassium voltage-gated channel subfamily J member 16(KCNJ16) |
| KCNJ16 | DQ892471 | Homo sapiens | potassium voltage-gated channel subfamily J member 16(KCNJ16) |
| KCNJ16 | DA109745 | Homo sapiens | potassium voltage-gated channel subfamily J member 16(KCNJ16) |
| KCNJ16 | NM_018658 | Homo sapiens | potassium voltage-gated channel subfamily J member 16(KCNJ16) |
| KCNJ16 | XM_017024609 | Homo sapiens | potassium voltage-gated channel subfamily J member 16(KCNJ16) |
| KCNJ16 | AF179353 | Homo sapiens | potassium voltage-gated channel subfamily J member 16(KCNJ16) |
| KCNJ16 | DA128508 | Homo sapiens | potassium voltage-gated channel subfamily J member 16(KCNJ16) |
| KCNJ16 | XM_017024610 | Homo sapiens | potassium voltage-gated channel subfamily J member 16(KCNJ16) |
| KCNJ16 | XM_017024613 | Homo sapiens | potassium voltage-gated channel subfamily J member 16(KCNJ16) |
| KCNJ16 | XM_017024611 | Homo sapiens | potassium voltage-gated channel subfamily J member 16(KCNJ16) |
| KCNJ16 | AK290799 | Homo sapiens | potassium voltage-gated channel subfamily J member 16(KCNJ16) |
| KCNJ16 | DA627411 | Homo sapiens | potassium voltage-gated channel subfamily J member 16(KCNJ16) |
| KCNJ16 | XM_017024612 | Homo sapiens | potassium voltage-gated channel subfamily J member 16(KCNJ16) |
| KCNJ16 | NM_001291625 | Homo sapiens | potassium voltage-gated channel subfamily J member 16(KCNJ16) |
| KCNJ16 | XM_011524781 | Homo sapiens | potassium voltage-gated channel subfamily J member 16(KCNJ16) |
| KCNJ16 | DA628604 | Homo sapiens | potassium voltage-gated channel subfamily J member 16(KCNJ16) |
| S100B | KU178320 | Homo sapiens | S100 calcium binding protein B(S100B) |
| S100B | EU176695 | Homo sapiens | S100 calcium binding protein B(S100B) |
| S100B | BC001766 | Homo sapiens | S100 calcium binding protein B(S100B) |
| S100B | BC041935 | Homo sapiens | S100 calcium binding protein B(S100B) |
| S100B | XM_017028424 | Homo sapiens | S100 calcium binding protein B(S100B) |
| S100B | AA310307 | Homo sapiens | S100 calcium binding protein B(S100B) |
| S100B | DQ892491 | Homo sapiens | S100 calcium binding protein B(S100B) |
| S100B | EB386131 | Homo sapiens | S100 calcium binding protein B(S100B) |
| S100B | KU178319 | Homo sapiens | S100 calcium binding protein B(S100B) |
| S100B | CR542123 | Homo sapiens | S100 calcium binding protein B(S100B) |
| S100B | NM_006272 | Homo sapiens | S100 calcium binding protein B(S100B) |
| CFC1 | BC069508 | Homo sapiens | cripto, FRL-1, cryptic family 1(CFC1) |
| CFC1 | NM_001270421 | Homo sapiens | cripto, FRL-1, cryptic family 1(CFC1) |
| CFC1 | CA777249 | Homo sapiens | cripto, FRL-1, cryptic family 1(CFC1) |
| CFC1 | NM_001270420 | Homo sapiens | cripto, FRL-1, cryptic family 1(CFC1) |
| CFC1 | BC146897 | Homo sapiens | cripto, FRL-1, cryptic family 1(CFC1) |
| CFC1 | CA941621 | Homo sapiens | cripto, FRL-1, cryptic family 1(CFC1) |
| CFC1 | AK290094 | Homo sapiens | cripto, FRL-1, cryptic family 1(CFC1) |
| CFC1 | BC074826 | Homo sapiens | cripto, FRL-1, cryptic family 1(CFC1) |
| CFC1 | CA948246 | Homo sapiens | cripto, FRL-1, cryptic family 1(CFC1) |
| CFC1 | BC110080 | Homo sapiens | cripto, FRL-1, cryptic family 1(CFC1) |
| CFC1 | BI793242 | Homo sapiens | cripto, FRL-1, cryptic family 1(CFC1) |
| CFC1 | BM310214 | Homo sapiens | cripto, FRL-1, cryptic family 1(CFC1) |
| CFC1 | NM_032545 | Homo sapiens | cripto, FRL-1, cryptic family 1(CFC1) |
| CFC1 | BG654700 | Homo sapiens | cripto, FRL-1, cryptic family 1(CFC1) |
| CFC1 | XM_011511486 | Homo sapiens | cripto, FRL-1, cryptic family 1(CFC1) |
| CFC1 | AK315326 | Homo sapiens | cripto, FRL-1, cryptic family 1(CFC1) |
| CFC1 | BC074825 | Homo sapiens | cripto, FRL-1, cryptic family 1(CFC1) |
| CFC1 | DQ786275 | Homo sapiens | cripto, FRL-1, cryptic family 1(CFC1) |
| CFC1 | AF312769 | Homo sapiens | cripto, FRL-1, cryptic family 1(CFC1) |
| CFC1 | BM509218 | Homo sapiens | cripto, FRL-1, cryptic family 1(CFC1) |
| COL3A1 | X14420 | Homo sapiens | collagen type III alpha 1 chain(COL3A1) |
| COL3A1 | X15332 | Homo sapiens | collagen type III alpha 1 chain(COL3A1) |
| COL3A1 | S79877 | Homo sapiens | collagen type III alpha 1 chain(COL3A1) |
| COL3A1 | X07240 | Homo sapiens | collagen type III alpha 1 chain(COL3A1) |
| COL3A1 | M11134 | Homo sapiens | collagen type III alpha 1 chain(COL3A1) |
| COL3A1 | M13146 | Homo sapiens | collagen type III alpha 1 chain(COL3A1) |
| COL3A1 | M59227 | Homo sapiens | collagen type III alpha 1 chain(COL3A1) |
| COL3A1 | AI755052 | Homo sapiens | collagen type III alpha 1 chain(COL3A1) |
| COL3A1 | BC028178 | Homo sapiens | collagen type III alpha 1 chain(COL3A1) |
| COL3A1 | NM_000090 | Homo sapiens | collagen type III alpha 1 chain(COL3A1) |
| COL3A1 | BP374999 | Homo sapiens | collagen type III alpha 1 chain(COL3A1) |
| COL3A1 | X06700 | Homo sapiens | collagen type III alpha 1 chain(COL3A1) |
| COL3A1 | X01655 | Homo sapiens | collagen type III alpha 1 chain(COL3A1) |
| COL3A1 | X01742 | Homo sapiens | collagen type III alpha 1 chain(COL3A1) |
| COL3A1 | AK091853 | Homo sapiens | collagen type III alpha 1 chain(COL3A1) |
| COL3A1 | AK308010 | Homo sapiens | collagen type III alpha 1 chain(COL3A1) |
| KRT19 | BC084574 | Homo sapiens | keratin 19(KRT19) |
| KRT19 | BG750971 | Homo sapiens | keratin 19(KRT19) |
| KRT19 | AB041267 | Homo sapiens | keratin 19(KRT19) |
| KRT19 | Y00503 | Homo sapiens | keratin 19(KRT19) |
| KRT19 | BP328445 | Homo sapiens | keratin 19(KRT19) |
| KRT19 | BG490639 | Homo sapiens | keratin 19(KRT19) |
| KRT19 | AB041268 | Homo sapiens | keratin 19(KRT19) |
| KRT19 | BC067744 | Homo sapiens | keratin 19(KRT19) |
| KRT19 | BC010409 | Homo sapiens | keratin 19(KRT19) |
| KRT19 | AK313261 | Homo sapiens | keratin 19(KRT19) |
| KRT19 | BC007628 | Homo sapiens | keratin 19(KRT19) |
| KRT19 | NM_002276 | Homo sapiens | keratin 19(KRT19) |
| KRT19 | BC002539 | Homo sapiens | keratin 19(KRT19) |
| KRT19 | DA068330 | Homo sapiens | keratin 19(KRT19) |
| KRT19 | DQ895373 | Homo sapiens | keratin 19(KRT19) |
| KRT19 | DQ892179 | Homo sapiens | keratin 19(KRT19) |
| DES | NM_001927 | Homo sapiens | desmin(DES) |
| DES | AK098332 | Homo sapiens | desmin(DES) |
| DES | U59167 | Homo sapiens | desmin(DES) |
| DES | AK022087 | Homo sapiens | desmin(DES) |
| DES | JX114780 | Homo sapiens | desmin(DES) |
| DES | AF055083 | Homo sapiens | desmin(DES) |
| DES | DQ104336 | Homo sapiens | desmin(DES) |
| DES | AK300654 | Homo sapiens | desmin(DES) |
| DES | DQ104337 | Homo sapiens | desmin(DES) |
| DES | EF617312 | Homo sapiens | desmin(DES) |
| DES | DQ104335 | Homo sapiens | desmin(DES) |
| DES | BQ941246 | Homo sapiens | desmin(DES) |
| DES | DQ895482 | Homo sapiens | desmin(DES) |
| DES | AF137053 | Homo sapiens | desmin(DES) |
| DES | AF167579 | Homo sapiens | desmin(DES) |
| DES | AY125465 | Homo sapiens | desmin(DES) |
| DES | AF487828 | Homo sapiens | desmin(DES) |
| DES | BC010072 | Homo sapiens | desmin(DES) |
| DES | AF521879 | Homo sapiens | desmin(DES) |
| DES | AF527578 | Homo sapiens | desmin(DES) |
| DES | AJ132926 | Homo sapiens | desmin(DES) |
| DES | AY083345 | Homo sapiens | desmin(DES) |
| DES | AF055081 | Homo sapiens | desmin(DES) |
| DES | AF055082 | Homo sapiens | desmin(DES) |
| DES | AK097038 | Homo sapiens | desmin(DES) |
| DES | BC032116 | Homo sapiens | desmin(DES) |
| DES | AF486807 | Homo sapiens | desmin(DES) |
| DES | DQ892282 | Homo sapiens | desmin(DES) |
| DES | AK123787 | Homo sapiens | desmin(DES) |
| DES | AL541778 | Homo sapiens | desmin(DES) |
| TTC6 | BC103914 | Homo sapiens | tetratricopeptide repeat domain 6(TTC6) |
| TTC6 | BC103915 | Homo sapiens | tetratricopeptide repeat domain 6(TTC6) |
| TTC6 | BC103916 | Homo sapiens | tetratricopeptide repeat domain 6(TTC6) |
| TTC6 | XR_001750287 | Homo sapiens | tetratricopeptide repeat domain 6(TTC6) |
| TTC6 | XM_011537430 | Homo sapiens | tetratricopeptide repeat domain 6(TTC6) |
| TTC6 | NM_001310135 | Homo sapiens | tetratricopeptide repeat domain 6(TTC6) |
| TTC6 | XM_011537431 | Homo sapiens | tetratricopeptide repeat domain 6(TTC6) |
| TTC6 | XM_017021254 | Homo sapiens | tetratricopeptide repeat domain 6(TTC6) |
| TTC6 | XM_017021255 | Homo sapiens | tetratricopeptide repeat domain 6(TTC6) |
| TTC6 | XR_943762 | Homo sapiens | tetratricopeptide repeat domain 6(TTC6) |
| TTC6 | XM_017021256 | Homo sapiens | tetratricopeptide repeat domain 6(TTC6) |
| TTC6 | XM_017021257 | Homo sapiens | tetratricopeptide repeat domain 6(TTC6) |
| TTC6 | XM_011537432 | Homo sapiens | tetratricopeptide repeat domain 6(TTC6) |
| TTC6 | BC038110 | Homo sapiens | tetratricopeptide repeat domain 6(TTC6) |
| TTC6 | BC014342 | Homo sapiens | tetratricopeptide repeat domain 6(TTC6) |
| TTC6 | BX161415 | Homo sapiens | tetratricopeptide repeat domain 6(TTC6) |
| TTC6 | NM_001007795 | Homo sapiens | tetratricopeptide repeat domain 6(TTC6) |
| PPP2R2B | DQ891785 | Homo sapiens | protein phosphatase 2 regulatory subunit Bbeta(PPP2R2B) |
| PPP2R2B | NM_001271899 | Homo sapiens | protein phosphatase 2 regulatory subunit Bbeta(PPP2R2B) |
| PPP2R2B | BI669304 | Homo sapiens | protein phosphatase 2 regulatory subunit Bbeta(PPP2R2B) |
| PPP2R2B | BP195204 | Homo sapiens | protein phosphatase 2 regulatory subunit Bbeta(PPP2R2B) |
| PPP2R2B | AK056192 | Homo sapiens | protein phosphatase 2 regulatory subunit Bbeta(PPP2R2B) |
| PPP2R2B | AK289717 | Homo sapiens | protein phosphatase 2 regulatory subunit Bbeta(PPP2R2B) |
| PPP2R2B | NM_001271948 | Homo sapiens | protein phosphatase 2 regulatory subunit Bbeta(PPP2R2B) |
| PPP2R2B | AK314810 | Homo sapiens | protein phosphatase 2 regulatory subunit Bbeta(PPP2R2B) |
| PPP2R2B | AL540315 | Homo sapiens | protein phosphatase 2 regulatory subunit Bbeta(PPP2R2B) |
| PPP2R2B | BC031790 | Homo sapiens | protein phosphatase 2 regulatory subunit Bbeta(PPP2R2B) |
| PPP2R2B | DA158596 | Homo sapiens | protein phosphatase 2 regulatory subunit Bbeta(PPP2R2B) |
| PPP2R2B | M64930 | Homo sapiens | protein phosphatase 2 regulatory subunit Bbeta(PPP2R2B) |
| PPP2R2B | DA155307 | Homo sapiens | protein phosphatase 2 regulatory subunit Bbeta(PPP2R2B) |
| PPP2R2B | NM_181677 | Homo sapiens | protein phosphatase 2 regulatory subunit Bbeta(PPP2R2B) |
| PPP2R2B | NM_181678 | Homo sapiens | protein phosphatase 2 regulatory subunit Bbeta(PPP2R2B) |
| PPP2R2B | DA365141 | Homo sapiens | protein phosphatase 2 regulatory subunit Bbeta(PPP2R2B) |
| PPP2R2B | NM_001271900 | Homo sapiens | protein phosphatase 2 regulatory subunit Bbeta(PPP2R2B) |
| PPP2R2B | NM_181675 | Homo sapiens | protein phosphatase 2 regulatory subunit Bbeta(PPP2R2B) |
| PPP2R2B | DQ894969 | Homo sapiens | protein phosphatase 2 regulatory subunit Bbeta(PPP2R2B) |
| PPP2R2B | NM_181676 | Homo sapiens | protein phosphatase 2 regulatory subunit Bbeta(PPP2R2B) |
| PPP2R2B | BX464346 | Homo sapiens | protein phosphatase 2 regulatory subunit Bbeta(PPP2R2B) |
| PPP2R2B | NM_181674 | Homo sapiens | protein phosphatase 2 regulatory subunit Bbeta(PPP2R2B) |
| PPP2R2B | DA522334 | Homo sapiens | protein phosphatase 2 regulatory subunit Bbeta(PPP2R2B) |
| PPP2R2B | AI621232 | Homo sapiens | protein phosphatase 2 regulatory subunit Bbeta(PPP2R2B) |
| PPP2R2B | DA523686 | Homo sapiens | protein phosphatase 2 regulatory subunit Bbeta(PPP2R2B) |
| PPP2R2B | NR_073527 | Homo sapiens | protein phosphatase 2 regulatory subunit Bbeta(PPP2R2B) |
| PPP2R2B | NR_073526 | Homo sapiens | protein phosphatase 2 regulatory subunit Bbeta(PPP2R2B) |
| PPP2R2B | AK294659 | Homo sapiens | protein phosphatase 2 regulatory subunit Bbeta(PPP2R2B) |
| PPP2R2B | BX647887 | Homo sapiens | protein phosphatase 2 regulatory subunit Bbeta(PPP2R2B) |
| PPP2R2B | AK295347 | Homo sapiens | protein phosphatase 2 regulatory subunit Bbeta(PPP2R2B) |
| PPP2R2B | AV726282 | Homo sapiens | protein phosphatase 2 regulatory subunit Bbeta(PPP2R2B) |
| PPP2R2B | DA533318 | Homo sapiens | protein phosphatase 2 regulatory subunit Bbeta(PPP2R2B) |
| PMP22 | CD519144 | Homo sapiens | peripheral myelin protein 22(PMP22) |
| PMP22 | BX464118 | Homo sapiens | peripheral myelin protein 22(PMP22) |
| PMP22 | DB466564 | Homo sapiens | peripheral myelin protein 22(PMP22) |
| PMP22 | AK300690 | Homo sapiens | peripheral myelin protein 22(PMP22) |
| PMP22 | D11428 | Homo sapiens | peripheral myelin protein 22(PMP22) |
| PMP22 | CR541931 | Homo sapiens | peripheral myelin protein 22(PMP22) |
| PMP22 | CR541953 | Homo sapiens | peripheral myelin protein 22(PMP22) |
| PMP22 | KR259964 | Homo sapiens | peripheral myelin protein 22(PMP22) |
| PMP22 | KR259963 | Homo sapiens | peripheral myelin protein 22(PMP22) |
| PMP22 | KR259962 | Homo sapiens | peripheral myelin protein 22(PMP22) |
| PMP22 | BQ232542 | Homo sapiens | peripheral myelin protein 22(PMP22) |
| PMP22 | BC019040 | Homo sapiens | peripheral myelin protein 22(PMP22) |
| PMP22 | BQ694106 | Homo sapiens | peripheral myelin protein 22(PMP22) |
| PMP22 | HY099893 | Homo sapiens | peripheral myelin protein 22(PMP22) |
| PMP22 | AK290640 | Homo sapiens | peripheral myelin protein 22(PMP22) |
| PMP22 | NR_104017 | Homo sapiens | peripheral myelin protein 22(PMP22) |
| PMP22 | NR_104018 | Homo sapiens | peripheral myelin protein 22(PMP22) |
| PMP22 | DQ895863 | Homo sapiens | peripheral myelin protein 22(PMP22) |
| PMP22 | BG424796 | Homo sapiens | peripheral myelin protein 22(PMP22) |
| PMP22 | CK818720 | Homo sapiens | peripheral myelin protein 22(PMP22) |
| PMP22 | DB504971 | Homo sapiens | peripheral myelin protein 22(PMP22) |
| PMP22 | NM_000304 | Homo sapiens | peripheral myelin protein 22(PMP22) |
| PMP22 | NM_153322 | Homo sapiens | peripheral myelin protein 22(PMP22) |
| PMP22 | DQ892623 | Homo sapiens | peripheral myelin protein 22(PMP22) |
| PMP22 | NM_001330143 | Homo sapiens | peripheral myelin protein 22(PMP22) |
| PMP22 | NM_153321 | Homo sapiens | peripheral myelin protein 22(PMP22) |
| PMP22 | L03203 | Homo sapiens | peripheral myelin protein 22(PMP22) |
| PMP22 | M94048 | Homo sapiens | peripheral myelin protein 22(PMP22) |
| PMP22 | DV460945 | Homo sapiens | peripheral myelin protein 22(PMP22) |
| PMP22 | XM_017024775 | Homo sapiens | peripheral myelin protein 22(PMP22) |
| PMP22 | HY119295 | Homo sapiens | peripheral myelin protein 22(PMP22) |
| PMP22 | NM_001281455 | Homo sapiens | peripheral myelin protein 22(PMP22) |
| PMP22 | XM_017024776 | Homo sapiens | peripheral myelin protein 22(PMP22) |
| PMP22 | BF725606 | Homo sapiens | peripheral myelin protein 22(PMP22) |
| PMP22 | NM_001281456 | Homo sapiens | peripheral myelin protein 22(PMP22) |
| PMP22 | X65968 | Homo sapiens | peripheral myelin protein 22(PMP22) |
| PMP22 | BQ212717 | Homo sapiens | peripheral myelin protein 22(PMP22) |
| PMP22 | BC091499 | Homo sapiens | peripheral myelin protein 22(PMP22) |
| PMP22 | AL833462 | Homo sapiens | peripheral myelin protein 22(PMP22) |
| PLP1 | M27110 | Homo sapiens | proteolipid protein 1(PLP1) |
| PLP1 | AK295374 | Homo sapiens | proteolipid protein 1(PLP1) |
| PLP1 | NM_000533 | Homo sapiens | proteolipid protein 1(PLP1) |
| PLP1 | DC342996 | Homo sapiens | proteolipid protein 1(PLP1) |
| PLP1 | BQ723748 | Homo sapiens | proteolipid protein 1(PLP1) |
| PLP1 | CR536542 | Homo sapiens | proteolipid protein 1(PLP1) |
| PLP1 | AK292728 | Homo sapiens | proteolipid protein 1(PLP1) |
| PLP1 | BX445448 | Homo sapiens | proteolipid protein 1(PLP1) |
| PLP1 | AK128782 | Homo sapiens | proteolipid protein 1(PLP1) |
| PLP1 | AV731932 | Homo sapiens | proteolipid protein 1(PLP1) |
| PLP1 | AI205189 | Homo sapiens | proteolipid protein 1(PLP1) |
| PLP1 | AK295388 | Homo sapiens | proteolipid protein 1(PLP1) |
| PLP1 | AK309239 | Homo sapiens | proteolipid protein 1(PLP1) |
| PLP1 | NM_001305004 | Homo sapiens | proteolipid protein 1(PLP1) |
| PLP1 | BQ717999 | Homo sapiens | proteolipid protein 1(PLP1) |
| PLP1 | M17085 | Homo sapiens | proteolipid protein 1(PLP1) |
| PLP1 | AK308966 | Homo sapiens | proteolipid protein 1(PLP1) |
| PLP1 | KU178261 | Homo sapiens | proteolipid protein 1(PLP1) |
| PLP1 | KU178262 | Homo sapiens | proteolipid protein 1(PLP1) |
| PLP1 | DC342197 | Homo sapiens | proteolipid protein 1(PLP1) |
| PLP1 | NM_001128834 | Homo sapiens | proteolipid protein 1(PLP1) |
| PLP1 | AK312340 | Homo sapiens | proteolipid protein 1(PLP1) |
| PLP1 | BC095452 | Homo sapiens | proteolipid protein 1(PLP1) |
| PLP1 | DA299940 | Homo sapiens | proteolipid protein 1(PLP1) |
| PLP1 | EB387051 | Homo sapiens | proteolipid protein 1(PLP1) |
| PLP1 | BC002665 | Homo sapiens | proteolipid protein 1(PLP1) |
| PLP1 | M54927 | Homo sapiens | proteolipid protein 1(PLP1) |
| PLP1 | CA389667 | Homo sapiens | proteolipid protein 1(PLP1) |
| PLP1 | NM_199478 | Homo sapiens | proteolipid protein 1(PLP1) |
| PLP1 | BT019602 | Homo sapiens | proteolipid protein 1(PLP1) |
| PLP1 | BT019601 | Homo sapiens | proteolipid protein 1(PLP1) |
| RMST | AF429306 | Homo sapiens | rhabdomyosarcoma 2 associated transcript (non-protein coding)(RMST) |
| RMST | AK056164 | Homo sapiens | rhabdomyosarcoma 2 associated transcript (non-protein coding)(RMST) |
| RMST | AF429305 | Homo sapiens | rhabdomyosarcoma 2 associated transcript (non-protein coding)(RMST) |
| RMST | NR_024037 | Homo sapiens | rhabdomyosarcoma 2 associated transcript (non-protein coding)(RMST) |
| SPC24 | BQ216591 | Homo sapiens | SPC24, NDC80 kinetochore complex component(SPC24) |
| SPC24 | XM_005259753 | Homo sapiens | SPC24, NDC80 kinetochore complex component(SPC24) |
| SPC24 | AY456387 | Homo sapiens | SPC24, NDC80 kinetochore complex component(SPC24) |
| SPC24 | NM_182513 | Homo sapiens | SPC24, NDC80 kinetochore complex component(SPC24) |
| SPC24 | BQ061822 | Homo sapiens | SPC24, NDC80 kinetochore complex component(SPC24) |
| SPC24 | XM_011527702 | Homo sapiens | SPC24, NDC80 kinetochore complex component(SPC24) |
| SPC24 | AK303157 | Homo sapiens | SPC24, NDC80 kinetochore complex component(SPC24) |
| SPC24 | AK075287 | Homo sapiens | SPC24, NDC80 kinetochore complex component(SPC24) |
| SPC24 | AK303266 | Homo sapiens | SPC24, NDC80 kinetochore complex component(SPC24) |
| SPC24 | BC105039 | Homo sapiens | SPC24, NDC80 kinetochore complex component(SPC24) |
| SPC24 | HY082333 | Homo sapiens | SPC24, NDC80 kinetochore complex component(SPC24) |
| SPC24 | NM_001317033 | Homo sapiens | SPC24, NDC80 kinetochore complex component(SPC24) |
| SPC24 | BC105037 | Homo sapiens | SPC24, NDC80 kinetochore complex component(SPC24) |
| SPC24 | NM_001317032 | Homo sapiens | SPC24, NDC80 kinetochore complex component(SPC24) |
| SPC24 | NM_001317031 | Homo sapiens | SPC24, NDC80 kinetochore complex component(SPC24) |
| ADGRL3 | AB018311 | Homo sapiens | adhesion G protein-coupled receptor L3(ADGRL3) |
| ADGRL3 | XM_017007942 | Homo sapiens | adhesion G protein-coupled receptor L3(ADGRL3) |
| ADGRL3 | XM_017007941 | Homo sapiens | adhesion G protein-coupled receptor L3(ADGRL3) |
| ADGRL3 | XM_017007940 | Homo sapiens | adhesion G protein-coupled receptor L3(ADGRL3) |
| ADGRL3 | XM_017007929 | Homo sapiens | adhesion G protein-coupled receptor L3(ADGRL3) |
| ADGRL3 | XM_011531791 | Homo sapiens | adhesion G protein-coupled receptor L3(ADGRL3) |
| ADGRL3 | AK294689 | Homo sapiens | adhesion G protein-coupled receptor L3(ADGRL3) |
| ADGRL3 | AK309793 | Homo sapiens | adhesion G protein-coupled receptor L3(ADGRL3) |
| ADGRL3 | AK299651 | Homo sapiens | adhesion G protein-coupled receptor L3(ADGRL3) |
| ADGRL3 | BC039452 | Homo sapiens | adhesion G protein-coupled receptor L3(ADGRL3) |
| ADGRL3 | DA808156 | Homo sapiens | adhesion G protein-coupled receptor L3(ADGRL3) |
| ADGRL3 | XM_017007934 | Homo sapiens | adhesion G protein-coupled receptor L3(ADGRL3) |
| ADGRL3 | AK000781 | Homo sapiens | adhesion G protein-coupled receptor L3(ADGRL3) |
| ADGRL3 | XM_017007933 | Homo sapiens | adhesion G protein-coupled receptor L3(ADGRL3) |
| ADGRL3 | XM_017007932 | Homo sapiens | adhesion G protein-coupled receptor L3(ADGRL3) |
| ADGRL3 | XM_017007931 | Homo sapiens | adhesion G protein-coupled receptor L3(ADGRL3) |
| ADGRL3 | XM_017007930 | Homo sapiens | adhesion G protein-coupled receptor L3(ADGRL3) |
| ADGRL3 | XM_017007939 | Homo sapiens | adhesion G protein-coupled receptor L3(ADGRL3) |
| ADGRL3 | XM_017007938 | Homo sapiens | adhesion G protein-coupled receptor L3(ADGRL3) |
| ADGRL3 | XM_017007937 | Homo sapiens | adhesion G protein-coupled receptor L3(ADGRL3) |
| ADGRL3 | XM_017007936 | Homo sapiens | adhesion G protein-coupled receptor L3(ADGRL3) |
| ADGRL3 | XM_017007935 | Homo sapiens | adhesion G protein-coupled receptor L3(ADGRL3) |
| ADGRL3 | NM_015236 | Homo sapiens | adhesion G protein-coupled receptor L3(ADGRL3) |
| ADGRL3 | NM_001322246 | Homo sapiens | adhesion G protein-coupled receptor L3(ADGRL3) |
| ADGRL3 | XM_011531788 | Homo sapiens | adhesion G protein-coupled receptor L3(ADGRL3) |
| ADGRL3 | NM_001322402 | Homo sapiens | adhesion G protein-coupled receptor L3(ADGRL3) |
| ADGRL3 | AK094703 | Homo sapiens | adhesion G protein-coupled receptor L3(ADGRL3) |
| ADGRL3 | BF511741 | Homo sapiens | adhesion G protein-coupled receptor L3(ADGRL3) |

### Methods

The present disclosure is also directed to methods of differentiating a pluripotent stem cell into a Schwann cell, the method comprising exposing an effective amount of a differentiator to a pluripotent stem cells for a time period sufficient to differentiate the cell into a Schwann cell.

The present disclosure is also directed to methods of culturing Schwann cells with a TGFP inhibitor. The present disclosure is also directed to methods of culturing Schwann cells, comprising exposing one or a plurality of cells to a tissue culture medium disclosed in any of the disclosed Tables. In some embodiments, the methods include exposing the cells to one or more tissue culture mediums for a time period of from about 1 day to about 20 days.

The disclosure also relates to a method of culturing Schwann cells in a system, the system comprising: (i) a cell culture vessel optionally comprising a hydrogel; (ii) one or a plurality of stem cells either in suspension or as a component of a spheroid; and (iii) one or plurality of differentiation factors. In some embodiments, the system further comprises one or combination of culture mediums disclosed herein. In some embodiments, the methods relate to replacing medium during a culture time of from about 12 to about 21 days at least one time to expose one or a plurality of stem cells to a cell medium for a time period sufficient to differentiate the one or plurality of stem cells into Schwann cells. In some embodiments, the system is free of or substantially free of feeder cells.

The disclosure provides improved methods for the derivation of enteric neural progenitors from human pluripotent stem cells (22). Many labs in the stem cell field no longer rely on the support of feeder cells and have adopted the use of defined basal media, such as mTeSR™1 (Stemcell Tech, 85850) or Essential 8 (Life Technologies, A2858501) for the maintenance of hPSC lines. Nevertheless, previous ENC induction methods commonly involve media containing serum replacement factors, namely knockout serum replacement (KSR), as is also the case in Comparative Example 2 (14, 20). In an effort to reduce the inconsistencies and quality control measures that undefined conditions may introduce to a protocol, optimizing ENC induction in minimal, chemically defined conditions, was pursued.

Recent studies have implemented alternative strategies for general NC induction using hPSCs, namely free floating embryoid body based approaches. The migratory cells that come as a result of embryoid body and subsequent neural rosette formations have been shown to be positive for neural crest specific markers SOX10, TFAP2A, BRN3A, ISL1 and ASCL1, and a subset found to be positive for regionally specific vagal markers HOXB2 and HOXB5, even without the inclusion of RA (23). Overall neural crest induction efficiency was assessed by FACS of p75 and HNK1 double positive cells, a strategy used to isolate NC cells in previous protocols (Lee et al. 2007). Results showed >60% induction efficiency in ES cell line H9 and across independent hiPSC lines. Enriched NC populations were then co-cultured with primary gut explants in a Transwell system to promote ENC identities enriched for HOXB2, HOXB3, HAND2 and EDNRB. Notably, this method incorporates brain-derived neurotrophic factor (BDNF), glial cell line-derived neurotrophic factor (GDNF), nerve growth factor (NGF), neurotrophin-3 (NT3) into culture conditions. A similar embryoid body approach incorporated brief exposure to RA during NC induction before eventually combining hPSC-derived NC cells with hPSC-derived intestinal organoids (HIOs) (24).

The disclosure relates to a method of differentiating a stem cell into a neural crest cell and then differentiating the NC cells into Schwann cells. In some embodiments the methods of culturing or differentiating into SCs comprise exposing a NC to BDNF, GDNF, NGF and/or NT3 for a time period sufficient to differentiate a stem cell into a NC. Some embodiments further comprise exposing one or a plurality of NCs to the differentiators identified above for a time period sufficient to differentiate into SCs. In some embodiments, the time period are sequential time periods in respect to more than one cell culture medium disclosed herein, wherein the cells are exposed to a first cell culture medium for a first time period, a second cell sulture medium for a second time period and a third cell culture medium for a third time period. As an example, in some embodiments, the cells are exposed sequentially in cell tissue medium to first, diffentiate stem cells into neural crest cells, and then neural crest cells into Schwann cells under conditions and for the time periods described in Example 2.

In some embodiments, the cells are cultured in a first and second step wherein the first or second step comprises two different cell culture media, and the first exposure to the first cell culture medium is for about 1, 2, 3, 4, 5, 6, 7, 8, 9, or about 10 or more days. In some embodiments, the second exposure to the second cell culture medium is for about 1, 2, 3, 4, 5, 6, 7, 8, 9, or about 10 or more days after exposure to the first cell culture medium.

In some embodiments, the cells are cultured in a first, second and third step wherein the first, second and third exposure step comprises three different cell culture media (one for each step), and the first exposure to the first cell culture medium is for about 1, 2, 3, 4, 5, 6, 7, 8, 9, or about 10 or more days. In some embodiments, the second exposure to the second cell culture medium is for about 1, 2, 3, 4, 5, 6, 7, 8, 9, or about 10 or more days after exposure to the first cell culture medium. In some embodiments, the second exposure to the third cell culture medium is for about 1, 2, 3, 4, 5, 6, 7, 8, 9, or about 10 or more days after exposure to the second cell culture medium.

### Screening Methods

The present disclosure also relates to a method of evaluating the toxicity of an agent comprising: (a) culturing one or more neuronal cells or Schwann cells on any of the devices disclosed herein; (b) exposing at least one agent to the one or more neuronal or Schwann cells; (c) measuring and/or observing one or more metrics of the neuronal or Schwann cell health; and (d) correlating one or more metrics of the one or more neuronal cells with the toxicity of the agent, such that, if the metrics are indicative of decreased cell viability, the agent is characterized as toxic and, if the metrics are indicative of unchanged or increased cell viability, the agent is characterized as non-toxic; wherein step (c) optionally comprises and/or observing one or more morphometric changes of the one or more neuronal cells or Schwann cells; and wherein step (d) optionally comprises correlating one or more morphometric changes of the one or more neuronal cells or Schwann cells with the toxicity of the agent, such that, if the changes are indicative of decreased cell viability, the agent is characterized as toxic and, if the changes are indicative of unchanged or increased cell viability, the agent is characterized as non-toxic. In some embodiments, the neuronal cells are any of the cells disclosed herein.

In some embodiments, the at least one agent comprises a small chemical compound. In some embodiments, the at least one agent comprises at least one environmental or industrial pollutant. In some embodiments, the at least one agent comprises one or a combination of small chemical compounds chosen from: chemotherapeutics, analgesics, cardiovascular modulators, cholesterol level modulators, neuroprotectants, neuromodulators, immunomodulators, anti-inflammatories, and anti-microbial drugs such as bacterial antibiotics. In some embodiments, the at least one agent comprises a therapeutically effective amount of an antibody, such as a clinically relevant monoclonal antibody such as Tysabri.

The present disclosure also relates to method of measuring the amount or degree of myelination or demyelination of one or more axons of one or a plurality of neuronal cells and/or one or a plurality of tissue explants, said method comprising: (a) culturing one or more neuronal cells and/or one or a plurality of tissue explants on any of the devices disclosed herein for a time and under conditions sufficient to grow at least one axon; (b) measuring and/or observing one or more morphometric changes of the one or more neuronal cells; and (c) correlating one or more morphometric changes of the one or more neuronal cells with a quantitative or qualitative change of myelination of the neuronal cells or tissue explants.

The present disclosure also relates to a method of measuring myelination or demyelination of one or more axons of one or a plurality of neuronal cells, said method comprising: (a) culturing one or more neuronal cells on any of the devices disclosed herein for a time and under conditions sufficient to grow at least one axon; (b) measuring and/or observing one or more physiological metrics of the one or more neuronal cells and/or one or more tissue explants; and (c) correlating one or more metrics of the one or more neuronal and/or one or more tissue explants cells with a quantitative or qualitative change of myelination of the neuronal cells; wherein step (b) optionally comprises and/or observing one or more morphometric changes of the one or more neuronal cells; and wherein step (c) optionally comprises correlating one or more morphometric changes of the one or more neuronal cells with the quantitative or qualitative change of myelination of the neuronal cells.

The present disclosure also relates to a method of measuring myelination or demyelination of one or more axons of one or a plurality of neuronal cells and/or one or a plurality of tissue explants, said method comprising: (a) culturing one or more neuronal cells and/or one or a plurality of tissue explants on any of the devices disclosed herein for a time and under conditions sufficient to grow at least one axon; and (b) detecting the amount of myelination on one or a plurality of axons of the one or more neuronal cells and/or one or more tissue explants. In each of the above embodiments, the step should comprise one or a plurality of SCs in culture with the neuronal cells or explants.

In some embodiments, the step of detecting the amount of myelination on one or a plurality of axons of the one or more neuronal cells and/or one or more tissue explants comprises exposing the cells to an antibody that binds to myelin.

In some embodiments, the method further comprises (i) exposing one or a plurality of neuronal cells and/or one or a plurality of tissue explants in the presence of SCs to at least one agent after steps (a) and (b); (ii) measuring and/or observing one or more expression patterns of the cells, measuring and/or observing one or more morphometric changes and/or detecting the quantitative amount of myelin from the one or a plurality of neuronal cells and/or one or a plurality of tissue explants; (iii) calculating a change of measurements, observations and/or quantitative amount of myelin from the one or a plurality of neuronal cells and/or the one or a plurality of tissue explants in the presence and absence of the agent; and (iv) correlating the change of measurements, observations and/or quantitative amount of myelin from the one or a plurality of neuronal cells and/or the one or a plurality of tissue explants to the presence or absence of the agent.

In some embodiments, the at least one agent comprises at least one environmental or industrial pollutant. In some embodiments, the at least one agent comprises one or a combination of small chemical compounds chosen from: chemotherapeutics, analgesics, cardiovascular modulators, cholesterol level modulators, neuroprotectants, neuromodulators, immunomodulators, anti-inflammatories, and anti-microbial drugs.

The present disclosure also relates to a method of measuring myelination or demyelination of one or more axons of one or a plurality of neuronal cells and/or one or a plurality of tissue explants, said method comprising: (a) culturing one or more neuronal cells and/or one or a plurality of tissue explants on any of the devices disclosed herein for a time and under conditions sufficient to grow at least one axon; and (b) inducing a compound action potential in such one or more neuronal cells and/or one or more tissue explants; (c) measuring the compound action potential; and (d) quantifying the levels of myelination of such one or more neuronal cells based on the presence of SCs in the culture. In some embodiments, the method further comprises exposing the one or more neuronal cells and/or one or a plurality of tissue explants to an agent. In some embodiments, the at least one agent comprises at least one environmental or industrial pollutant.

In some embodiments, the at least one agent comprises one or a combination of small chemical compounds chosen from: chemotherapeutics, analgesics, cardiovascular modulators, cholesterol level modulators, neuroprotectants, neuromodulators, immunomodulators, anti-inflammatories, and anti-microbial drugs.

In some embodiments, the at least one agent comprises a small chemical compound. In some embodiments, the at least one agent comprises at least one environmental or industrial pollutant. In some embodiments, the at least one agent comprises one or a combination of small chemical compounds chosen from: chemotherapeutics, analgesics, cardiovascular modulators, cholesterol, neuroprotectants, neuromodulators, immunomodulators, anti-inflammatories, and anti-microbial drugs.

In some embodiments, the at least one agent comprises one or a combination of chemotherapeutics chosen from: Actinomycin, Alitretinoin, All-trans retinoic acid, Azacitidine, Azathioprine, Bexarotene, Bleomycin, Bortezomib, Capecitabine, Carboplatin, Chlorambucil, Cisplatin, Cyclophosphamide, Cytarabine, Dacarbazine (DTIC), Daunorubicin, Docetaxel, Doxifluridine, Doxorubicin, Epirubicin, Epothilone, Erlotinib, Etoposide, Fluorouracil, Gefitinib, Gemcitabine, Hydroxyurea, Idarubicin, Imatinib, Irinotecan, Mechlorethamine, Melphalan, Mercaptopurine, Methotrexate, Mitoxantrone, Nitrosoureas, Oxaliplatin, Paclitaxel, Pemetrexed, Romidepsin, Tafluposide, Temozolomide (Oral dacarbazine), Teniposide, Tioguanine (formerly Thioguanine), Topotecan, Tretinoin, Valrubicin, Vemurafenib, Vinblastine Vincristine, Vindesine, Vinorelbine, Vismodegib, and Vorinostat.

In some embodiments, the at least one agent comprises one or a combination of analgesics chosen from: Paracetoamol, Non-steroidal anti-inflammatory drugs (NSAIDs), COX-2 inhibitors, opioids, flupirtine, tricyclic antidepressants, carbamaxepine, gabapentin, and pregabalin.

In some embodiments, the at least one agent comprises one or a combination of cardiovascular modulators chosen from: nepicastat, cholesterol, niacin, Scutellaria, prenylamine, dehydroepiandrosterone, monatepil, esketamine, niguldipine, asenapine, atomoxetine, flunarizine, milnacipran, mexiletine, amphetamine, sodium thiopental, flavonoid, bretylium, oxazepam, and honokiol.

In some embodiments, the at least one agent comprises one or a combination of neuroprotectants and/or neuromodulators chosen from: tryptamine, galanin receptor 2, phenylalanine, phenethylamine, N-methylphenethylamine, adenosine, kyptorphin, substance P, 3-methoxytyramine, catecholamine, dopamine, GAB A, calcium, acetylcholine, epinephrine, norepinephrine, and serotonin.

In some embodiments, the at least one agent comprises one or a combination of immunomodulators chosen from: clenolizimab, enoticumab, ligelizumab, simtuzumab, vatelizumab, parsatuzumab, Imgatuzumab, tregalizaumb, pateclizumab, namulumab, perakizumab, faralimomab, patritumab, atinumab, ublituximab, futuximab, and duligotumab.

In some embodiments, the at least one agent comprises one or a combination of anti-inflammatories chosen from: ibuprofen, aspirin, ketoprofen, sulindac, naproxen, etodolac, fenoprofen, diclofenac, flurbiprofen, ketorolac, piroxicam, indomethacin, mefenamic acid, meloxicam, nabumetone, oxaprozin, ketoprofen, famotidine, meclofenamate, tolmetin, and salsalate.

In some embodiments, the at least one agent comprises one or a combination of anti-microbials chosen from: antibacterials, antifungals, antivirals, antiparasitics, heat, radiation, and ozone.

The present disclosure also relates to a method of detecting and/or quantifying neuronal cell growth viability comprising: (a) quantifying one or a plurality of neuronal cells; (b) culturing the one or more neuronal cells on any of the systems disclosed herein; and (c) calculating the number of neuronal cells in the composition after culturing for a time period sufficient to allow growth of the one or plurality of cells. In some embodiments, step (c) comprises detecting an internal and/or external recording of such one or more neuronal cells after culturing one or more neuronal cells and correlating the recording with a measurement of the same recording corresponding to a known or control number of cells. In any of the methods, a system comprising the one or plurality of neuronal cells also comprises one or a combination of: Low Passage Mature SCs, High Passage Mature SCs, Myelinating SCs or SCPDs.

In some embodiments, the method further comprises contacting the one or more neuronal cells to one or more agents. In some embodiments, the method further comprises: (i) measuring an intracellular and/or extracellular expression of nucleic acid sequences or protein before and after the step of contacting the one or more neuronal cells and/or SCs to the one or more agents; and (ii) correlating the difference in the expression before contacting the one or more neuronal cells and/or SCs to the one or more agents to the expression after contacting the one or more neuronal cells or SCs to the one or more agents to a change in cell number. Expression profiling can be completed by quantitative or semi-quantitative PCR.

### Methods of Treating

The present disclosure is also directed to methods of transplanting a composition comprising Schwann cells into a subject in need thereof by administering a pharmaceutical composition described herein.

In another aspect, the present disclosure relates to a method of treating a spinal cord injury or peripheral neuropathy in a subject in need thereof, the method comprising administering to the subject a therapeutically effective amount of the Schwann cells disclosed herein.

In another aspect, the present disclosure relates to a method of treating diabetic peripheral neuropathy (DPN), Charcot-Marie-Tooth disease (CMT), amyotrophic lateral sclerosis (ALS), neuroinflammation, Guillain-Barr syndrome, radiation-induced nerve damage and/or chemotherapeutic-induced nerve damage; the method comprising administering to a subject in need of treatment a therapeutically effective amount of an agent identified by any of the screening methods disclosed herein. In some embodiments, the method comprises administering to a subject in need of treatment a therapeutically effective amount of one or a combination of the cell compositions disclosed herein. In some embodiments, the cell compositions comprise one or a plurality of low-passage Schwann cells, high-passage Schwann cells, SCPD cells or myelinating Schwann cells. In some embodiments, the cells are derived from pluripotent stem cells and/or neural crest cells.

In another aspect, the present disclosure relates to a subject comprising any one of the compositions of any of the Schwann cells disclosed herein. In some embodiments, the subject is a mammal. In some embodiments, the subject is a human.

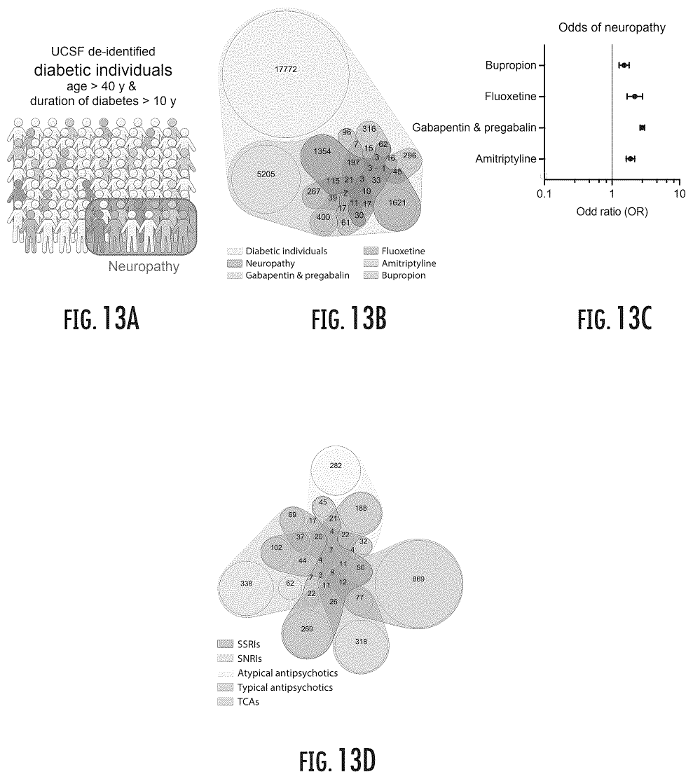

The disclosure also relates to a method of treating diabetic peripheral neuropathy in a subject in need thereof comprising administering to the subject a therapeutically effective amount of an agent or a pharmaceutical composition comprising a therapeutically effective amount of any one or combination of disclosed Schwann cells and a pharmaceutically acceptable carrier. In some embodiments, the agent is chosen from an agent of Table S4 or FIG. 13A through 13D. In some embodiments, the agent is chosen from an agent of Table S4 or a pharmaceutically acceptable salt or derivative thereof. In some embodiments, the agent is chosen from an agent of Table S4, or a pharmaceutically acceptable salt or derivative thereof, that comprises a Z score above 1.0, 1.1, 1.2, 1.3, 1.4, 1.5, 1.6, 1.7, 1.8, 1.9, or above 2.0.

The disclosure also relates to a method of treating Charcot-Marie-Tooth disease (CMT) in a subject in need thereof comprising administering to the subject a therapeutically effective amount of an agent or a pharmaceutical composition comprising a therapeutically effective amount of any one or combination of disclosed Schwann cells and a pharmaceutically acceptable carrier. In some embodiments, the agent is chosen from an agent of Table S4. In some embodiments, the agent is chosen from an agent of Table S4 or a pharmaceutically acceptable salt or derivative thereof. In some embodiments, the agent is chosen from an agent of Table S4, or a pharmaceutically acceptable salt or derivative thereof, that comprises a Z score above 1.0, 1.1, 1.2, 1.3, 1.4, 1.5, 1.6, 1.7, 1.8, 1.9, or above 2.0.

The disclosure also relates to a method of treating amyotrophic lateral sclerosis (ALS) in a subject in need thereof comprising administering to the subject a therapeutically effective amount of an agent or a pharmaceutical composition comprising a therapeutically effective amount of any one or combination of disclosed Schwann cells and a pharmaceutically acceptable carrier. In some embodiments, the agent is chosen from an agent of Table S4. In some embodiments, the agent is chosen from an agent of Table S4 or a pharmaceutically acceptable salt or derivative thereof. In some embodiments, the agent is chosen from an agent of Table S4, or a pharmaceutically acceptable salt or derivative thereof, that comprises a Z score above 1.0, 1.1, 1.2, 1.3, 1.4, 1.5, 1.6, 1.7, 1.8, 1.9, or above 2.0.

The disclosure also relates to a method of treating neuroinflammation in a subject in need thereof comprising administering to the subject a therapeutically effective amount of an agent or a pharmaceutical composition comprising a therapeutically effective amount of any one or combination of disclosed Schwann cells and a pharmaceutically acceptable carrier. In some embodiments, the agent is chosen from an agent of Table S4. In some embodiments, the agent is chosen from an agent of Table S4 or a pharmaceutically acceptable salt or derivative thereof. In some embodiments, the agent is chosen from an agent of Table S4, or a pharmaceutically acceptable salt or derivative thereof, that comprises a Z score above 1.0, 1.1, 1.2, 1.3, 1.4, 1.5, 1.6, 1.7, 1.8, 1.9, or above 2.0.

The disclosure also relates to a method of treating Guillain-Barr syndrome in a subject in need thereof comprising administering to the subject a therapeutically effective amount of an agent or a pharmaceutical composition comprising a therapeutically effective amount of any one or combination of disclosed Schwann cells and a pharmaceutically acceptable carrier. In some embodiments, the agent is chosen from an agent of Table S4. In some embodiments, the agent is chosen from an agent of Table S4 or a pharmaceutically acceptable salt or derivative thereof. In some embodiments, the agent is chosen from an agent of Table S4, or a pharmaceutically acceptable salt or derivative thereof, that comprises a Z score above 1.0, 1.1, 1.2, 1.3, 1.4, 1.5, 1.6, 1.7, 1.8, 1.9, or above 2.0.

The disclosure also relates to a method of treating radiation-induced nerve damage and/or chemotherapeutic-induced nerve damage in a subject in need thereof comprising administering to the subject a therapeutically effective amount of an agent or a pharmaceutical composition comprising a therapeutically effective amount of any one or combination of disclosed Schwann cells and a pharmaceutically acceptable carrier. In some embodiments, the agent is chosen from an agent of Table S4. In some embodiments, the agent is chosen from an agent of Table S4 or a pharmaceutically acceptable salt or derivative thereof. In some embodiments, the agent is chosen from an agent of Table S4, or a pharmaceutically acceptable salt or derivative thereof, that comprises a Z score above 1.0, 1.1, 1.2, 1.3, 1.4, 1.5, 1.6, 1.7, 1.8, 1.9, or above 2.0.

In some embodiments, the methods of treating utilize one or more active agents listed in Table S4, or pharmaceutically acceptable salt(s) thereof. In some embodiments, the methods of treating utilize an active agent with a Z score of 2.0 or more, or pharmaceutically acceptable salt(s) thereof. In some embodiments, the active agent is Bupropion or a pharmaceutically acceptable salt thereof. In some embodiments, the active agent is Cyclopenthiazide or a pharmaceutically acceptable salt thereof. In some embodiments, the active agent is Niridazole or a pharmaceutically acceptable salt thereof. In some embodiments, the active agent is Gliquidone or a pharmaceutically acceptable salt thereof. In some embodiments, the active agent is Condelphine or a pharmaceutically acceptable salt thereof. In some embodiments, the active agent is Furaltadone or a pharmaceutically acceptable salt thereof. In some embodiments, the active agent is Furaltadone hydrochloride. In some embodiments, the active agent is Captopril or a pharmaceutically acceptable salt thereof. In some embodiments, the active agent is Nimesulide or a pharmaceutically acceptable salt thereof. In some embodiments, the active agent is Nafronyl or a pharmaceutically acceptable salt thereof. In some embodiments, the active agent is Nafronyl oxalate. In some embodiments, the active agent is Tolbutamide or a pharmaceutically acceptable salt thereof. In some embodiments, the active agent is Perindopril or a pharmaceutically acceptable salt thereof. In some embodiments, the active agent is Trolox or a pharmaceutically acceptable salt thereof. In some embodiments, the active agent is Spaglumic acid or a pharmaceutically acceptable salt thereof. In some embodiments, the active agent is (d,l)-Tetrahydroberberine or a pharmaceutically acceptable salt thereof. In some embodiments, the active agent is Ribavirin or a pharmaceutically acceptable salt thereof. In some embodiments, the active agent is Triamcinolone or a pharmaceutically acceptable salt thereof.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g., symptoms of a gut motility disorder), kind of concurrent treatment, complications from the disease being treated or other health-related problems. Other therapeutic regimens or agents can be used in conjunction with the methods and compounds of Applicants' disclosure. Adjustment and manipulation of established dosages (e.g., frequency and duration) are well within the ability of those skilled in the art.

The Schwann cells of the disclosure can be administered alone or can be coadministered to the patient. Coadministration is meant to include simultaneous or sequential administration of the compound individually or in combination (more than one compound or agent). Thus, the preparations can also be combined, when desired, with other active substances (e.g., to reduce metabolic degradation). The compositions of the present disclosure can be delivered by transdermally, by a topical route, formulated as applicator sticks, solutions, suspensions, emulsions, gels, creams, ointments, pastes, jellies, paints, powders, and aerosols. Liquid form preparations include solutions, suspensions, and emulsions, for example, water or water/propylene glycol solutions. The compositions of the present disclosure may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present disclosure can also be delivered as microspheres for slow release in the body. For example, microspheres can be administered via intravenous injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995). In some embodiments, the formulations of the compositions of the present disclosure can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present disclosure into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present disclosure can also be delivered as nanoparticles.

The disclosure also relates to method of treating DPN in a subject in need thereof comprising administering to the subject a pharmaceutical composition comprising a therapeutically effective amount of buproprion or a pharmaceutically acceptable salt thereof or derivative thereof; and a pharmaceutically acceptable carrier. Buproprion comprises the formula of:

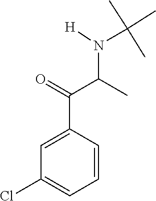 [SMILES: CC(NC(C)(C)C)C(=O)c1cccc(Cl)c1]

Other embodiments are described in the following non-limiting Examples. Various publications, including patents, published applications, GenBank accession numbers, technical articles and scholarly articles are cited throughout the specification. Each of these cited publications is incorporated by reference herein in its entirety.

### EXAMPLES

Examples 1 and 2 were carried out with methods including, but not limited to, the following:

### Example 1

Derivation and Prospective Isolation of SC Lineages from hPSCs

We previously established hPSC differentiation protocols to access various NC lineages including enteric and sensory neurons (Barber et al., 2019; Chambers et al., 2012; Fattahi et al., 2016; Tchieu et al., 2017). However, there are currently no methods for the efficient derivation of authentic Schwann cells from hPSCs. Our past efforts of deriving SCs relied on the prolonged, 2¬3 months, culture of NC-enriched progenitor cells to obtain a small proportion of gliogenic cells (Lee et al., 2007). More studies reported on the derivation of SC-like cells from hPSCs but did not show molecular authenticity through gene expression profiling and failed to demonstrate functional myelination (Huang et al., 2017; Kim et al., 2017; Liu et al., 2012; Ziegler et al., 2011). While the mechanisms of SC specification during human development remain to be elucidated, SCs are thought to arise from SOX10+ NC cells in a stepwise process. Based on studies in the mouse and chick embryos, NC first gives rise to SC precursors that are competent to associate with neuronal fiber bundles in the developing nerves. The associated neurons produce NRG1 which promotes the survival and further differentiation of SC precursors (SCPs) by activating ERBB3 receptors (Newbern and Birchmeier, 2010). By E13.5 of mouse development, SC precursors give rise to immature SCs which express lineage-specific markers such as GFAP, S100 and POU3F1 while maintaining the expression of SOX10. Terminal differentiation of SCs into myelinating and non-myelinating fates continues for extended time periods and concludes only after birth (Jessen et al., 2015).

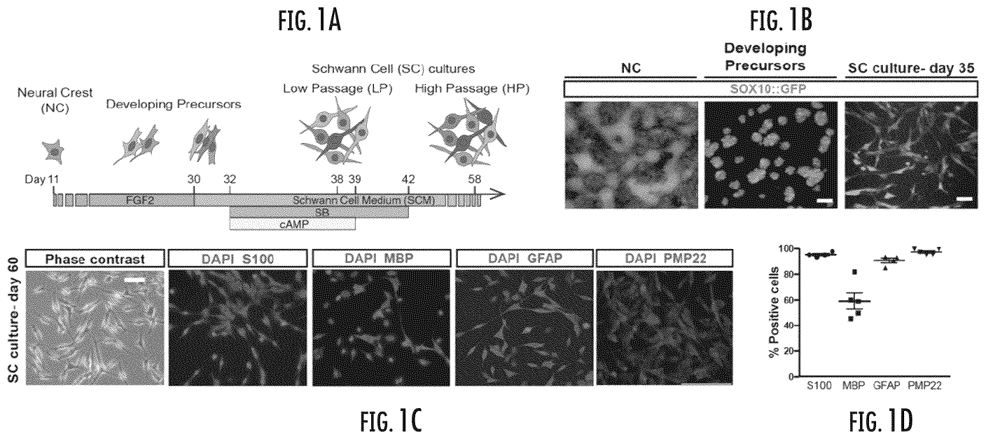

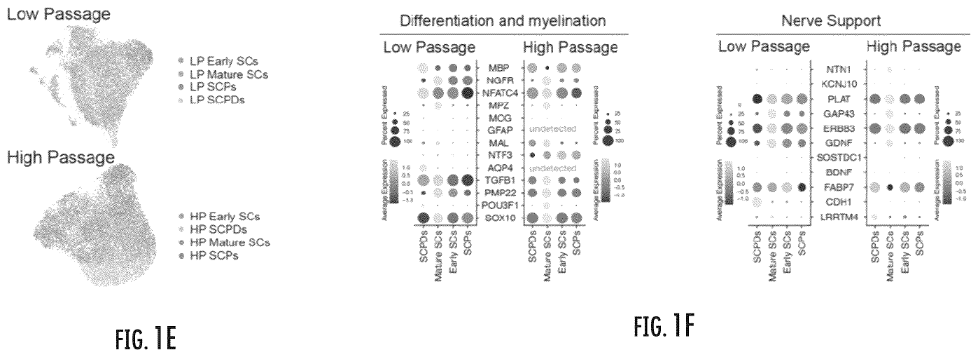

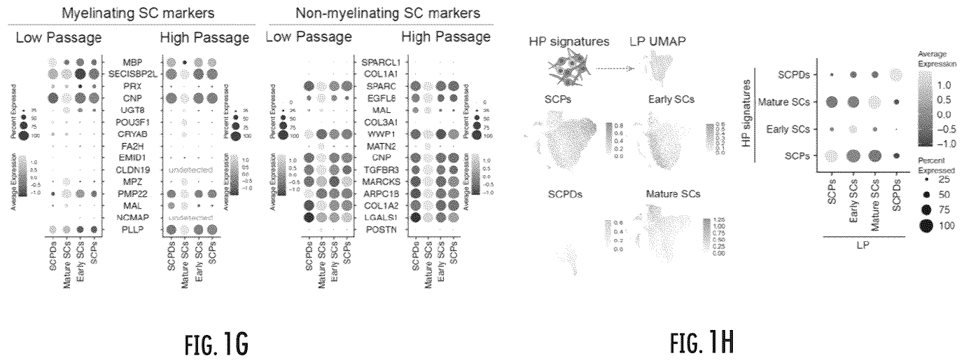

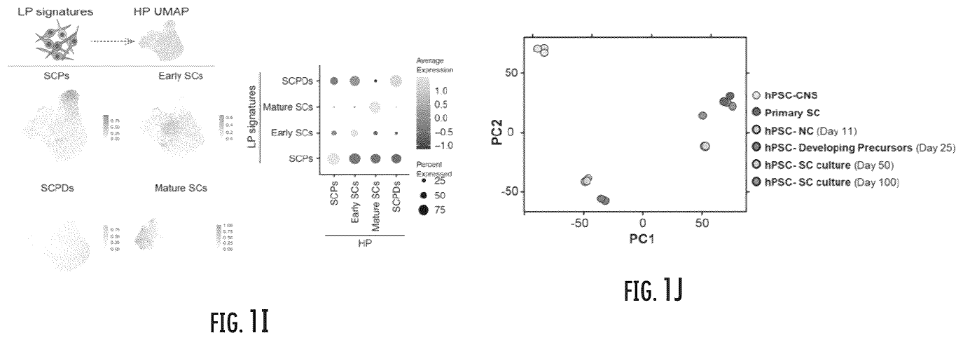

Initial hPSC-based NC differentiation protocols relied on the delamination of putative NC cells from neuroepithelial lineages combined with the prospective isolation of p75+ and/or HNK1+ NC precursors (Bajpai et al., 2010; Lee et al., 2007). While those protocols yield various NC-derived lineages, the levels of SOX10 expression are generally low. In contrast, more directed NC induction protocols based on timed exposure to activators of WNT signaling show robust induction of SOX10 in the majority of cells by day 11 of differentiation (Barber et al., 2019; Fattahi et al., 2016; Menendez et al., 2011; Mica et al., 2013; Tchieu et al., 2017). Upon further culture, those hPSC-derived NC cells can be directed into SOX10+ melanocytes (Mica et al., 2013) but also give rise to SOX10-mesenchymal and neuronal precursors (Fattahi et al., 2016; Lee et al., 2007; Mica et al., 2013; Tchieu et al., 2017). Since SOX10 is a key marker in the SC lineage (Finzsch et al., 2010), we first screened for conditions that maintained its expression in cultured NC precursors. We determined the percentage of SOX10+ cells in 2D or 3D NC cultures in the presence of modulators of EGF, FGF, WNT, Notch, TGFβ, BMP and endothelin 3 signaling. We observed that activation of WNT signaling through CHIR99021 exposure and treatment with FGF2 resulted in the maintenance of SOX10 expression in 3D aggregates that we refer to as developing precursors (FIG. 1 A and B).

Further treatment of developing precursors with Schwann cell media (SCM) containing FGF2, SB431542 and dbcAMP enabled the induction of additional SC markers such as GFAP, POU3F1, PMP22, MBP, AQP4, and MPZ and upregulation of genes involved in glial-neuronal interactions and support including GDNF, ERBB3, and GAP43 among others (FIG. 5 A and B). These cultures can be passaged and maintained for several weeks while retaining the expression of key SC markers S100, MBP, GFAP and PMP22 (FIG. 1 C and D).

In order to determine the cellular diversity of our hPSC-derived SC cultures, we performed single cell RNA-sequencing (scRNA-seq) at two differentiation time points; low passage (LP, day 38) and high passage (HP, day 58). Unbiased clustering of both LP and HP datasets revealed four transcriptionally distinct cell types namely SCPs, early SCs, mature SCs and SCP derivatives (SCPDs) (FIG. 1E and FIG. S2A). These cell types differentially expressed canonical markers of SC differentiation and function (FIG. 1F). For example, mature SCs in both LP and HP cultures expressed higher levels of myelinating and non-myelinating SC markers such as PMP22 and NGFR. Nerve support markers and neurotrophic factors including ERBB3, GDNF NGF, BDNF and GAP43 were also enriched in mature SCs particularly in the HP cultures (FIG. 1F and FIG. S2B). We module scored the LP and HP cell types for a number of SC functional gene sets including neurotrophic factors, neurotransmitter receptors and transcription factors (Table S1) and detected differential expression of many neurotransmitter receptors and postsynaptic signal transmission genes by our Schwann cell types (FIG. S2C). We also identified transcription factors that were specifically expressed by each population in LP and HP cultures. For example, early SCs were enriched for E2F7 and E2F8 while mature SCs expressed SOX10 and FOX01, POU3F2, TBX19 more specifically. POU6F2 was the common enriched transcription factor in LP and HP SCPDs (FIG. S2D).

TABLE S1

| Neurotrophic | Neurotransmitter | Transcription | Neurotrophic | Neurotransmitter | Transcription |
|---|---|---|---|---|---|
| Factors | Receptors | Factors | Factors | Receptors | Factors |
| BDNF | ACTN2 | SRY | IFNA6 | AP2M1 | SOX14 |
| NGF | ADCY1 | SOX1 | IFNA7 | AP2S1 | SOX15 |
| NTF3 | ADCY2 | SOX2 | IFNA8 | APBA1 | SOX17 |
| NTF4 | ADCY3 | SOX3 | IFNA10 | ARHGEF7 | SOX18 |
| CNTF | ADCY4 | SOX4 | IFNA13 | ARHGEF9 | SOX21 |
| LIF | ADCY5 | SOX5 | IFNA14 | CACNG2 | SOX30 |
| IL6 | ADCY6 | SOX6 | IFNA16 | CACNG3 | POU1F1 |
| PRL | ADCY7 | SOX7 | IFNA17 | CACNG4 | POU2F1 |
| GH1 | ADCY8 | SOX8 | IFNA21 | CACNG8 | POU2F2 |
| LEP | ADCY9 | SOX9 | IFNB1 | CALM1 | POU2F3 |
| IFNA1 | AKAP5 | SOX10 | IFNG | CAMK1 | POU3F1 |
| IFNA2 | AP2A1 | SOX11 | OSM | CAMK2A | POU3F2 |
| IFNA4 | AP2A2 | SOX12 | GDNF | CAMK2B | POU3F3 |
| IFNA5 | AP2B1 | SOX13 | ARTN | CAMK2D | POU3F4 |
| PSPN | CAMK4 | POU4F2 | NRTN | CAMK2G | POU4F1 |
| EFNA1 | CAMKK1 | POU4F3 | FGF14 | GABRR1 | FOXJ1 |
| EFNA2 | CAMKK2 | POU5F1 | FGF15 | GABRR2 | FOXJ2 |
| EFNA3 | CASK | POU5F1P1 | FGF16 | GABRR3 | FOXJ3 |
| EFNA4 | CHRNA1 | POU5F1P3 | FGF17 | GIT1 | FOXK1 |
| EFNA5 | CHRNA2 | POU5F1P4 | FGF18 | GLRA1 | FOXK2 |
| EFNB1 | CHRNA3 | POU5F2 | FGF19 | GLRA2 | FOXM1 |
| EFNB2 | CHRNA4 | POU6F1 | FGF20 | GLRA3 | FOXN1 |
| EFNB3 | CHRNA5 | POU6F2 | FGF21 | GLRB | FOXN2 |
| EGF | CHRNA6 | PAX1 | FGF22 | GNAI1 | FOXN3 |
| NRG1 | CHRNA7 | PAX2 | FGF23 | GNAI2 | FOXN4 |
| NRG2 | CHRNA9 | PAX3 | PDGF | GNAI3 | FOXO1 |
| NRG3 | CHRNB2 | PAX4 | ADCYAP1 | GNAL | FOXO3 |
| NRG4 | CHRNB3 | PAX5 | IL1 | GNAT3 | FOXO4 |
| TGFA | CHRNB4 | PAX6 | IL2 | GNB1 | FOXO6 |
| TGBF1 | CHRND | PAX7 | IL3 | GNB2 | FOXP1 |
| TGFB2 | CHRNE | PAX8 | IL5 | GNB3 | FOXP2 |
| TGFB3 | CHRNG | PAX9 | CXCL8 | GNB4 | FOXP3 |
| GMFB | CREB1 | FOXA1 | CSF1 | GNB5 | FOXP4 |
| INS | DLG1 | FOXA2 | CSF2 | GNG10 | FOXQ1 |
| IGF1 | DLG2 | FOXA3 | CXCL1 | GNG11 | FOXR1 |
| VEGFA | DLG3 | FOXB1 | | GNG12 | FOXR2 |
| VEGFB | DLG4 | FOXB2 | | GNG13 | TBXT |
| VEGFC | EPB41L1 | FOXC1 | | GNG2 | TBR1 |
| VEGFD | ERBB4 | FOXC2 | | GNG3 | TBX1 |
| PGF | GABBR1 | FOXD1 | | GNG4 | TBX2 |
| FGF1 | GABBR2 | FOXD2 | | GNG5 | TBX3 |
| FGF2 | GABRA1 | FOXD3 | | GNG7 | TBX4 |
| FGF3 | GABRA2 | FOXD4 | | GNG8 | TBX5 |
| FGF4 | GABRA3 | FOXD5 | | GNGT1 | TBX6 |
| FGF5 | GABRA4 | FOXE1 | | GNGT2 | TBX10 |
| FGF6 | GABRA5 | FOXE2 | | GRIA1 | TBX15 |
| FGF7 | GABRA6 | FOXE3 | | GRIA2 | TBX18 |
| FGF8 | GABRB1 | FOXF1 | | GRIA3 | TBX19 |
| FGF9 | GABRB2 | FOXF2 | | GRIA4 | TBX20 |
| FGF10 | GABRB3 | FOXG1 | | GRIK1 | TBX21 |
| FGF11 | GABRG2 | FOXH1 | | GRIK2 | TBX22 |
| FGF12 | GABRG3 | FOXI1 | | GRIK3 | HOXA1 |
| FGF13 | GABRQ | FOXI2 | | GRIK4 | HOXA2 |
| | GRIN1 | HOXA4 | | GRIK5 | HOXA3 |
| | GRIN2A | HOXA5 | | NEFL | RUNX2 |
| | GRIN2B | HOXA6 | | NPTN | RUNX3 |
| | GRIN2C | HOXA7 | | NRAS | E2F1 |
| | GRIN2D | HOXA8 | | NRG1 | E2F2 |
| | GRIN3A | HOXA9 | | NRGN | E2F3 |
| | GRIN3B | HOXA10 | | NSF | E2F4 |
| | GRIP1 | HOXA11 | | PDPK1 | E2F5 |
| | GRIP2 | HOXA12 | | PICK1 | E2F6 |
| | HRAS | HOXA13 | | PLCB1 | E2F7 |
| | HTR3A | HOXB1 | | PLCB2 | E2F8 |
| | HTR3B | HOXB2 | | PLCB3 | EHF |
| | HTR3C | HOXB3 | | PPM1E | ELF1 |
| | HTR3D | HOXB4 | | PPM1F | ELF2 |
| | HTR3E | HOXB5 | | PRKAA1 | ELF3 |
| | KCNJ10 | HOXB6 | | PRKAA2 | ELF4 |
| | KCNJ12 | HOXB7 | | PRKAB1 | ELF5 |
| | KCNJ15 | HOXB8 | | PRKAB2 | ELK1 |
| | KCNJ16 | HOXB9 | | PRKACA | ELK3 |
| | KCNJ2 | HOXB13 | | PRKACB | ELK4 |
| | KCNJ3 | HOXC4 | | PRKACG | ERF |
| | KCNJ4 | HOXC5 | | PRKAG1 | ERG |
| | KCNJ5 | HOXC6 | | PRKAG2 | ETS1 |
| | KCNJ6 | HOXC8 | | PRKAG3 | ETS2 |
| | KCNJ9 | HOXC9 | | PRKARIA | ETV1 |
| | KIF17 | HOXC10 | | PRKARIB | ETV2 |
| | KPNA2 | HOXC11 | | PRKAR2A | ETV3 |
| | KRAS | HOXC12 | | PRKAR2B | ETV3L |
| | LIN7A | HOXC13 | | PRKCA | ETV4 |
| | LIN7B | HOXD1 | | PRKCB | ETV5 |
| | LIN7C | HOXD3 | | PRKCG | ETV6 |
| | LRRC7 | HOXD4 | | PRKX | ETV7 |
| | MAPK1 | HOXD8 | | RAC1 | FEV |
| | MAPK3 | HOXD9 | | RASGRF1 | FLI1 |
| | MAPT | HOXD10 | | RASGRF2 | GABPA |
| | MDM2 | HOXD11 | | RPS6KA1 | SPDEF |
| | MYO6 | HOXD12 | | RPS6KA2 | SPIB |
| | NBEA | HOXD13 | | RPS6KA3 | SPIC |
| | NCALD | RUNX1 | | RPS6KA6 | SPI1 |
| | TSPAN7 | FOSB | | SRC | FOS |
| | TUBA1A | FOSL1 | | TUBAL3 | |
| | TUBA1B | FOSL2 | | TUBB1 | |
| | TUBA1C | JUN | | TUBB2A | |
| | TUBA3C | JUNB | | TUBB2B | |
| | TUBA3D | JUND | | TUBB3 | |
| | TUBA3E | | | TUBB4A | |
| | TUBA4A | | | TUBB4B | |
| | TUBA4B | | | TUBB6 | |
| | TUBA8 | | | TUBB8 | |
| | | | | TUBB8B | |

To validate the authenticity of our hPSC-derived SCs, we assessed the expression of top differentially expressed myelinating and non-myelinating Schwann cell specific genes derived from a primary mouse single cell transcriptomics dataset previously published by Segal and colleagues (Tasdemir-Yilmaz et al., 2021) (FIG. 1G, Table S2). We detected the expression of these markers in both our LP and HP cultures with cell type specific expression patterns (FIG. CG). Some markers such as MPZ and MATN2 were specifically expressed by a single cluster namely mature SCs. However, the majority of other genes showed differential transcript levels between cell types while not exclusively expressed in a single population (FIG. G). Interestingly, our LP and HP mature SC populations were highly enriched for both myelinating and non-myelinating markers indicating that our hPSC-derived SCs reliably express markers of authentic SCs.

TABLE S2

| mySC.DE15 | nmSC.DE15 |
|---|---|
| Mpz | Col1a2 |
| Cnp | Sparc |
| Prx | Col3a1 |
| Mbp | Col1a1 |
| Pmp22 | Fam198b |
| Cldn19 | Postn |
| Secisbp21 | Marcks |
| Emid1 | Egfl8 |
| Mal | Tgfbr3 |
| Pllp | Fabp7 |
| Fa2h | Matn2 |
| Cryab | Sparcl1 |
| Pou3f1 | Lgals1 |
| Ncmap | Arpc1b |
| Ptn | Wwp1 |
| Ugt8a | Cnp |
| Gjc3 | Mal |
| Fabp7 | Col5a1 |
| Eif1 | Nid2 |
| Arhgap19 | Mpz |
| Csrp2 | Gas2l3 |
| Cd9 | Cond1 |
| Smtn | Ank3 |
| Qk | Lrrc8b |
| Dbi | Itga6 |
| Kcna1 | Col5a2 |
| Bcas1 | Sbspon |
| Ncam1 | Vgl13 |
| Ednrb | Vim |
| Mag | Slc35f1 |
| Peli2 | Ahnak |
| Cdkn1a | Nrp2 |
| S100a6 | Ntrk3 |
| Postn | Epb41l4b |
| Fxyd6 | Lama2 |
| Ptprz1 | Ngfr |
| Utrn | Vwa1 |
| Ybx1 | Lamb1 |
| Rasal2 | Csrp1 |
| Marcks | Scn7a |
| Mgll | Pmp22 |
| Elovl1 | Cryab |
| Plekhb1 | Npr3 |
| Mlip | Epha5 |
| Kras | Rarres2 |
| Itih5 | Serpinh1 |
| Sostdc1 | Megf9 |
| Atp1b3 | Lamc1 |
| Bzw2 | Epb41l3 |
| Art3 | Nr2f2 |
| Fut8 | Art3 |
| Gypc | Akap12 |
| Dynll1 | Mbnl2 |
| Apoe | L1cam |
| Ndrg1 | Cond2 |
| Ttyh1 | mt-Co2 |
| Spag9 | Abca8b |
| Sfrp1 | Ctnnd2 |
| Egr2 | Gjc3 |
| Fbln2 | Ubl3 |
| Gldn | Glul |
| Tmem134 | mt-Co3 |
| Cdh2 | Tpt1 |
| Cdkn1b | Mcam |
| Mpp6 | Cadm4 |
| Stk39 | Entpd2 |
| Mdk | Lama4 |
| Kmt5a | Pdgfa |
| Fam178b | Tuba1a |
| Cdkn1c | Sh3pxd2a |
| Kcna2 | mt-Co1 |
| Zfp36l1 | Klf6 |
| Slc36a2 | S100a6 |
| Pla2g16 | Cnn3 |
| Ctnnd2 | Tead1 |
| Slc35f1 | Timp2 |
| Col4a1 | Tubb2b |
| Ndrg2 | Arpc1a |
| Nr2f1 | Chl1 |
| Gnas | Rhobtb3 |
| Gas5 | Gulp1 |
| Fyn | Sash1 |
| Arpc1a | Rap2a |
| Tsc22d4 | Gatm |
| Aatk | Sostdc1 |
| Mt1 | Lhfpl2 |
| Adamts5 | mt-Cytb |
| Cst3 | Rap1a |
| Fnta | Dab2 |
| Ptgfrn | Plp1 |
| Metrnl | Epas1 |
| Pmepa1 | Loxl2 |
| Sparc | mt-Nd1 |
| Maf | 2310022B05Rik |
| Lmo4 | Mbp |
| Ogn | Nid1 |
| Mest | mt-Nd3 |
| Sema3b | Lgi1 |
| Zdhhc2 | Serpine2 |
| mt-Co3 | Rsu1 |
| Pxdn | Anxa5 |
| Cald1 | Col18a1 |
| Ptpre | Fth1 |
| Ttf1 | Cdh11 |
| Cnn3 | Angptl2 |
| Gng2 | Eef1a1 |
| Fryl | Selm |
| Cadm1 | Mdk |
| Mapre2 | Matn4 |
| Drp2 | Anxa2 |
| Kif1a | S1pr3 |
| Id3 | Celf2 |
| Ldhb | Fign |
| Hey2 | Arl6ip1 |
| Mmd2 | Add3 |
| Gas2l3 | Itgb1 |
| Limch1 | Sat1 |
| Vim | Zeb2 |
| Matn2 | Deptor |
| H19 | Tmod2 |
| Adgrg6 | Itm2a |
| Rhob | Tax1bp3 |
| Sema6d | Spata6 |
| mt-Nd4 | Sema6d |
| St6gal1 | S100a10 |
| Lasp1 | Col15a1 |
| Atp1a1 | Prnp |
| Sh3bp5 | Rab31 |
| Dusp15 | Hbegf |
| Shtn1 | Lpar4 |
| Vdac2 | Cadm1 |
| Dag1 | Fbxo7 |
| Agrn | Cdh19 |
| Col4a2 | Itgav |
| Entpd2 | Mest |
| Kank4 | Insc |
| Tmem50a | Hey2 |
| Cdk6 | Ltbp4 |
| Oat | Prkcq |
| Elovl5 | Mmp2 |
| Cdh11 | Jam3 |
| Me1 | Tmem123 |
| Rps9 | Ldhb |
| mt-Nd1 | Fkbp9 |
| Sirt2 | Ppic |
| Rnf7 | Afap1l2 |
| Stxbp6 | 1500015O10Rik |
| Gatm | Tpm4 |
| Slitrk2 | Tmsb10 |
| Epas1 | Cdkn1c |
| Rgcc | Gas7 |
| Ppm1l | Ldha |
| Tpt1 | Vcl |
| Ivns1abp | Adam23 |
| Nov | Id3 |
| Efhd1 | Ednrb |
| Nr2f2 | Ptgfrn |
| Ahr | Ahr |
| Epb41l2 | mt-Atp6 |
| Abhd4 | Anxa6 |
| Tead1 | Pcdh9 |
| Ypel2 | Ptrf |
| 2700094K13Rik | Rab32 |
| Abca8b | Stard13 |
| Mras | Maf |
| Itgb8 | Secisbp2l |
| Ado | Col27a1 |
| Fxyd1 | Slc22a23 |
| Prss35 | Gata2 |
| Vat1l | Sema3b |
| Serinc5 | Thbs2 |
| Wbp5 | Oaf |
| Fzd2 | Fmo1 |
| S100b | Gsn |
| Tmem189 | Prss23 |
| Rbms1 | Kcna6 |
| Gja1 | Dbi |
| Meg3 | Ywhaz |
| Vcl | Ctsc |
| Ick | mt-Nd2 |
| Ostf1 | Pten |
| Jag1 | Tubb2a |
| Rhoc | Tsc22d4 |
| Tspan7 | Marveld1 |
| Osbpl1a | Igfbp4 |
| Calm2 | Prss35 |
| Lrrc8b | Cst3 |
| Rbms3 | Dlc1 |
| Sh3gl3 | Cdh1 |
| Sema5a | Pdpn |
| Sox2 | 2700094K13Rik |
| Alcam | Fabp5 |
| Myh10 | Kank4 |
| Tjp2 | Nr4a1 |
| Megf10 | Rpl30 |
| Fgfr1 | Me1 |
| Lpcat1 | Capn6 |
| Ccser2 | Mprip |
| Col1a1 | Emp1 |
| Grb10 | Fxyd6 |
| Acsbg1 | Itgb3 |
| Sema3c | App |
| Mmp14 | Rian |
| Usp6nl | Bcan |
| Sgk1 | Fbln2 |
| Ctnnal1 | Scarb2 |
| Alad | Itga4 |
| Tspan3 | Serinc5 |
| Deb1 | Jag1 |
| Fstl1 | Pja2 |
| Snx16 | Sema3c |
| Gnai1 | Meg3 |
| Map4 | Cd59a |
| Maged1 | Cyr61 |
| Igf2 | Hspa1b |
| Taldo1 | Itgb8 |
| Frmd3 | Adgrg6 |
| Dst | Mlip |
| Sh3pxd2a | Tenm3 |
| Sparcl1 | mt-Nd4 |
| Spop | Cd81 |
| Sulf2 | Parva |
| Cfl2 | Srgap1 |
| Gpr155 | Nrarp |
| Cux1 | Cdk6 |
| Purb | Dab1 |
| Calr | Rpl5 |
| Mbnl2 | Ivns1abp |
| Daam2 | Dusp6 |
| Lpar4 | Itpr3 |
| Gm28989 | Cttn |
| Lpar1 | 2-Sep |
| Peg3 | Jun |
| Foxd3 | Adgrg1 |
| Top2a | Fkbp1a |
| Itpkb | Rack1 |
| Abca1 | Flrt3 |
| Fkbp1a | Rab38 |
| Epb41l3 | Plxnb3 |
| Hsp90ab1 | Rhob |
| Tnc | Slc12a2 |
| Col11a1 | B2m |
| Phip | Ckap4 |
| Trim2 | Prx |
| Hspa12a | Pebp1 |
| Hnrnpa3 | Hspa1a |
| Plekha1 | Adgra3 |
| Wsb2 | Tgfb2 |
| Adam10 | Pxdn |
| Pcdh10 | Rnd3 |
| Kit | Sox11 |
| Slc22a17 | Kmt5a |
| Notch1 | Hs3st1 |
| Itga6 | Rpl7 |
| Spon1 | Serinc3 |
| Ftl1 | Tma7 |
| Fam126a | Copa |
| Nrep | Alcam |
| Foxp2 | Dmd |
| Tuba1b | Fgfr1 |
| Dclk1 | Rpl13 |
| Igf1r | Tmem229a |
| Plpp1 | Bnip3l |
| Lifr | Erbb3 |
| Fzd8 | Bach1 |
| Nfia | Pkig |
| Slc3a2 | P4hb |
| Samd8 | H2afz |
| Ak3 | Hdac4 |
| Rps27rt | Rps18 |
| Slc6a8 | Kcna2 |
| Dock9 | Sox10 |
| Tex2 | Col16a1 |
| Elmo1 | Klf13 |
| Cd47 | Fxyd1 |
| Slc9a3r1 | Rasgef1c |
| Metrn | Sptbn1 |
| Matn4 | Ndst1 |
| Srp14 | Dynlrb1 |
| Fam102a | Mmp15 |
| Mat2a | Crym |
| Timp2 | Sar1a |
| Arhgap24 | Gja1 |
| Mfap3l | Eif4g2 |
| Magt1 | Kit |
| Reep5 | Ncam1 |
| Ttyh3 | Scd2 |
| mt-Co1 | Taf13 |
| Sdcbp | Sh3gl3 |
| Tcf4 | Ptn |
| Ubb | Capn5 |
| Slc44a1 | Aspa |
| Luzp2 | Lmna |
| Tmod2 | Ilk |
| Emp2 | Hspa5 |
| Vldlr | Plscr4 |
| Scg3 | Rps3a1 |
| Shroom2 | Capg |
| Fgf1 | Jund |
| Sccpdh | Oaz1 |
| Iqgap1 | Dusp3 |
| Bcan | Top2a |
| Laptm4b | Nme2 |
| Ptpdc1 | S100a11 |
| Tenm3 | Racgap1 |
| Pdpn | Eif3h |
| Fth1 | Prss12 |
| Gal3st1 | Dclk1 |
| Prss12 | Hey1 |
| Ercc6l | Eif4a1 |
| Atp6voc | Cdo1 |
| Mcm6 | Hist1h1c |
| Plp1 | Col5a3 |
| Chl1 | Rpl8 |
| Crip1 | Laptm4b |
| Prrg3 | Vapa |
| Axl | Rpl4 |
| Auts2 | Ctnna1 |
| Gamt | Slc22a17 |
| Sertad1 | Plat |
| Prdx1 | Uaca |
| Aldh2 | Gfra3 |
| 2310022B05Rik | Sox2 |
| Atp1b2 | Flna |
| Ppp1r14c | Rgcc |
| Igfbp4 | Agrn |
| Elovl6 | Sncg |
| Nfasc | Ube2c |
| Kif5b | Gnb2 |
| Fabp5 | Cdc42 |
| Gng5 | Dhh |
| Rnf128 | Ptprg |
| Sox10 | Wipi1 |
| Rpl15 | Ccnb2 |
| Mtss11 | Mmd2 |
| H2afy | Ppip5k2 |
| Il16 | Reep3 |
| Camk2d | Slc7a2 |
| Hsd17b11 | Aatk |
| Maged2 | Arl8a |
| Arl6ip1 | Cenpa |
| Spred1 | Cap1 |
| Cd200 | Ccna2 |
| Kcnk1 | Gng12 |
| Tspan17 | Parp14 |
| Clic1 | Pcdh10 |
| Cpe | Egln1 |
| Pdlim4 | Nedd4 |
| Mapk8ip1 | Lgi4 |
| Eef2 | Id2 |
| Dpysl2 | Pcdh7 |
| Sox11 | Qk |
| Msi2 | Snx16 |
| Hmgb2 | Ugt8a |
| Mme | Stmn1 |
| Dnajc9 | Nfe2l2 |
| Rbp1 | Reep5 |
| Sema6a | Rtn1 |
| Nr4a2 | Fam19a5 |
| Dusp16 | Gltp |
| Tmed10 | Npdc1 |
| Lmnb1 | Edi1 |
| Idi1 | Iqgap2 |
| Fam212b | Nap1l1 |
| Abca2 | Akap2 |
| Tmem64 | Slc25a3 |
| Fmo1 | Pcbp1 |
| Nudt3 | Clmn |
| Csrp1 | Slc1a4 |
| Lmna | Pdx1 |
| Prkaca | Tnc |
| Hey1 | Zfp536 |
| Slc43a3 | Kcna1 |
| Nkain4 | Ctnnal1 |
| Cdh19 | Clcn3 |
| Rplp2 | Ptprz1 |
| Fign | Pnrc1 |
| Kif21a | H2afy |
| Srsf2 | Ddx3x |
| Rmnd5a | Pou3f1 |
| Aif1l | Add1 |
| Eid1 | Mxi1 |
| Mdh1 | Atp1b1 |
| Mfap2 | Sfr1 |
| Col2a1 | Srp54b |
| Rab14 | Lpar1 |
| Atp5b | Rhbdf1 |
| Ngfr | Hipk3 |
| Smco3 | Rnaset2b |
| Rpl6 | Ccnb1 |
| Cab39l | Clcn5 |
| Adamts15 | Birc5 |
| Cdk1 | Wnt6 |
| Shc4 | Crtap |
| Ccdc34 | Rtkn |
| Actb | Mgst1 |
| Hnrnpd | Shroom2 |
| Ahnak | Atp1a1 |
| Snrpg | Rps9 |
| Cbx5 | Fmnl2 |
| H2afv | Rac1 |
| Hnrnpa2b1 | Pmepa1 |
| H3f3a | Jam2 |
| Ran | Ucp2 |
| Igsf3 | Pfn1 |
| Cers2 | Myl12a |
| Gpr137b | Foxd3 |
| Sat1 | Dlk1 |
| Filip1 | Daam2 |
| Hnrnpa0 | Itih5 |
| Frmd6 | Fibin |
| Bri3 | Spcs1 |
| Azin1 | Tubb5 |
| Luc712 | Lrrtm4 |
| Trib2 | Msi2 |
| Cd164 | Igfbp7 |
| Depdc7 | Purg |
| Zfp3612 | Heyl |
| Utp111 | Peg3 |
| Ctsk | Clic1 |
| Ctsd | Serp1 |
| Nkain3 | Rbms1 |
| Ccna2 | Ybx1 |
| Lama4 | Cdc42ep4 |
| Ndufv2 | Cdca8 |
| Enah | Sqle |
| Nrarp | Rps25 |
| Sh3d19 | Fndc3b |
| Ptms | Rhou |
| Sfxn1 | Sh3bp5 |
| Mif | Itm2c |
| Smc2 | Rpl10 |
| Arpc3 | H2afx |
| Csdc2 | Capns1 |
| Med131 | Maged2 |
| Plce1 | Cdc42se1 |
| Il6st | St3gal2 |
| B2m | Nras |
| Olfml2a | Bgn |
| Ddah2 | Mrpl33 |
| Rps3 | Rps27l |
| Acadl | Rplp1 |
| Ngfrap1 | Rab5a |
| Col16a1 | Myl6 |
| Ptprg | Ppp1r18 |
| Tpx2 | Clic4 |
| Ubl3 | Tmem65 |
| Ssrp1 | Rcn3 |
| Lhfp | Arl2bp |
| Smpdl3a | Gm17541 |
| Pcbp4 | Phgdh |
| Cd81 | Morf4l2 |
| Igsf8 | Fos |
| Gm17541 | Plekha1 |
| Tax1bp1 | Fstl1 |
| Rcn2 | Plekhb1 |
| Timm8b | Epb41l4a |
| Mob3b | H19 |
| Neat1 | Arrdc3 |
| Hdac4 | Cdk1 |
| Acvr2a | Vwc2 |
| Slco3a1 | Aldoc |
| Wwc2 | Il1rap |
| Col1a2 | Ctdsp2 |
| Pygb | Socs2 |
| Foxo1 | Rpl23 |
| Serinc1 | Smpdl3a |
| Racgap1 | Lgr4 |
| 1810058I24Rik | Mapre2 |
| Dynlrb1 | Plk1 |
| Hnrnph1 | Rxrg |
| Tkt | Rpia |
| Nedd4 | Rpl37 |
| Flna | Tm7sf3 |
| Mthfd21 | Snca |
| 1190002N15Rik | Bcl7a |
| Rbbp7 | Prelid1 |
| Otud7b | Tpx2 |
| Sh3pxd2b | Luzp2 |
| Trim28 | Luc7l3 |
| 2810417H13Rik | Tuba1c |
| Calm1 | Tmem234 |
| Lgr4 | Atpif1 |
| Atp6v0e | Txndc17 |
| Cd151 | Fzd2 |
| Hsdl2 | Rps4x |
| Ucp2 | Actg1 |
| Rpn1 | Rabac1 |
| Dusp6 | Cxcr4 |
| Ppp1cc | Cbx5 |
| Rnf13 | Emp3 |
| Glg1 | Emp2 |
| Socs2 | Hcfc1r1 |
| Crabp2 | Pard6g |
| Nipal1 | Col2a1 |
| Rplp1 | Actb |
| Akt1 | H2afv |
| Gpx4 | Rnf130 |
| Cdh1 | Ywhaq |
| Sec11c | P4ha1 |
| Map2 | Thbd |
| Fmnl2 | Ctsk |
| Psip1 | Rpl27a |
| Tcf7l1 | Park7 |
| Sema4c | Casp12 |
| Smc4 | Ube2d2a |
| Fjx1 | Rab18 |
| Morf4l2 | Acsbg1 |
| Tmpo | |
| S1pr3 | |
| Hnrnpa1 | |
| Jak1 | |
| Itch | |
| Kirrel | |
| Xpo1 | |
| Plxnb2 | |
| Ctnnbip1 | |
| Marcksl1 | |
| Heyl | |
| Bhmt2 | |
| Col15a1 | |
| Frmd4a | |
| Itm2b | |
| Palld | |
| 6330403K07Rik | |
| Dazap2 | |
| Rabac1 | |
| Crabp1 | |
| Gpx1 | |
| Arpc5 | |
| Mcm4 | |
| Gm20594 | |
| Cdc42ep1 | |
| Syt11 | |
| Tgfbr3 | |
| Ap3s1 | |
| Rps26 | |
| Srsf1 | |
| Tubb4b | |
| Kcna6 | |
| Rab2a | |
| Crym | |
| Cox8a | |
| Slc1a4 | |
| Tnfaip8l1 | |
| Ank2 | |
| Laptm4a | |
| Pbx1 | |
| Anp32b | |
| Rad21 | |
| Lsamp | |
| Fus | |
| Gm8797 | |
| Stard13 | |
| Ank3 | |
| Fam129a | |
| Hist1h2bc | |
| Ddx5 | |
| Ube2c | |
| Bag5 | |
| Fbxo7 | |
| Gabarapl2 | |
| Gnptab | |
| Ywhab | |
| Rtkn | |
| Ceacam10 | |
| Rpl36 | |
| Cdk19 | |
| Notch3 | |
| Ncaph | |
| Fubp1 | |
| Idh2 | |
| Selm | |
| Rrm2 | |
| Ckb | |
| Aldoc | |
| Ubxn4 | |
| Cadm3 | |
| Cdc42ep2 | |
| Tcte2 | |
| Emp3 | |
| Rnf103 | |
| Dek | |
| Ccdc62 | |
| Lrrtm4 | |
| Pkm | |
| Cerk | |
| Ogt | |
| Anp32a | |
| mt-Nd2 | |
| Smox | |
| Ust | |
| Rhbdf1 | |
| Fads1 | |
| Gfra2 | |
| Hnrnpdl | |
| H2afx | |
| Sptlc2 | |
| Lmo1 | |
| Impad1 | |
| Ltbp4 | |
| Ddn | |
| Scand1 | |
| Rpl13a | |
| Rap1gap | |
| Mapk3 | |
| Imp3 | |
| Rps8 | |
| Gpc6 | |
| Gtf2i | |
| Mtmr2 | |
| Hmox1 | |
| Phyh | |
| Lect1 | |
| Tprg1 | |
| Fzd7 | |
| Spin1 | |
| Itga4 | |
| Nid2 | |
| Alg2 | |
| Wwp1 | |
| Lcorl | |
| Hcfc1 | |
| Hid1 | |
| Flrt3 | |
| Mt2 | |
| Mir99ahg | |
| Spcs1 | |
| Lzts2 | |
| Adgra3 | |
| S100a4 | |
| Itm2c | |
| Fxyd3 | |
| Cts1 | |
| Cltb | |
| Id4 | |
| Cks1b | |
| Srebf1 | |
| Sumo3 | |
| Nek1 | |
| Kif19a | |
| Fmn2 | |
| Dpysl3 | |
| Tmem38b | |
| Ephb6 | |
| Col9a2 | |
| Saraf | |
| Npepps | |
| Tagln2 | |
| Ddx17 | |
| Eif5 | |
| Arf6 | |
| Dkk3 | |
| H2-Q4 | |
| Mex3a | |
| Egfl8 | |
| Ddit4 | |
| Birc5 | |
| Rps29 | |
| Ccnb1 | |
| St5 | |
| Mprip | |
| Twsg1 | |
| Plekhf1 | |
| Dlk1 | |
| Tubb5 | |
| Pabpn1 | |
| Ecscr | |
| Angptl2 | |
| Tmem165 | |
| Mad2l1 | |
| Sae1 | |
| Itgav | |
| Samhd1 | |
| Rian | |
| Ntrk2 | |
| Cks2 | |
| Nid1 | |
| Ube2e3 | |
| Rab7b | |
| Gramd1b | |
| Pag1 | |
| Myl6 | |
| Gabarap | |
| Dhrs1 | |
| Rps16 | |
| Polr3h | |
| Smc3 | |
| Nav1 | |
| Tia1 | |
| Hmgcr | |
| Anapc5 | |
| Tyro3 | |
| Slc38a2 | |
| Ncapd2 | |
| Dusp18 | |
| Phldb2 | |
| Ralbp1 | |
| Spata13 | |
| Las1l | |
| Fam83f | |
| Gpsm2 | |
| Tuba1c | |
| Zfand5 | |
| Ubxn6 | |
| H2afz | |
| Rasa3 | |
| Baiap2 | |
| Nudt4 | |
| Lrrc4c | |
| Higd2a | |
| Gid4 | |
| Hnrnpr | |
| Mcam | |
| Anln | |
| Anks1 | |
| Pcbp2 | |
| Neo1 | |
| Reep3 | |
| Lrrtm1 | |
| Nt5de2 | |
| Tbc1d10a | |
| Cdo1 | |
| Ddhd1 | |
| Cdk2 | |
| Pik3ip1 | |
| Rasgef1a | |
| Plekha7 | |
| Pik3r1 | |
| Chd3 | |
| Hmgcs2 | |
| Cacng4 | |
| Dad1 | |
| Cyr61 | |
| Tm7sf3 | |
| Slc6a1 | |
| Gas1 | |
| Pdrg1 | |
| Sbds | |
| Fam53b | |
| Ndn | |
| Rtn4 | |
| Ndufb9 | |
| Dmd | |
| Fignl1 | |
| Arl14ep | |
| Dnajc10 | |
| Tmcc3 | |
| Csad | |
| Col26a1 | |
| Rpl5 | |
| Gpc1 | |
| Ptpra | |
| Mgat3 | |
| Twf1 | |
| Atp6v1e1 | |
| Bche | |
| Lurap1 | |
| Tead2 | |
| Sdc2 | |
| Nfib | |
| Klhl13 | |
| Kpna2 | |
| Tmsb4x | |
| Cyfip1 | |
| Mlec | |
| Rnd3 | |
| 1810011O10Rik | |
| Gng11 | |
| Pds5a | |
| Fbxl5 | |
| Arhgef28 | |
| Spg20 | |
| Sfpq | |
| Zfhx3 | |
| Rhbdf2 | |
| Klf9 | |
| Lamp1 | |
| Rpl13 | |
| Adgrg1 | |
| Capg | |
| Numb | |
| Gapdh | |
| Tex40 | |
| Rab31 | |
| Fcgrt | |
| Npr3 | |
| Gm7609 | |
| Vangl2 | |
| BC051077 | |
| Reck | |
| Calm3 | |
| Cdca8 | |
| Arf4 | |
| Vgll3 | |
| Dennd2a | |
| Als2 | |
| Eif2s2 | |
| St13 | |
| Tmem176b | |
| Vegfa | |
| Bcas2 | |
| Rps25 | |
| Rai14 | |
| Cenpa | |
| Rxrg | |
| Mvb12b | |
| Golga3 | |
| Rarres2 | |
| Emp1 | |
| Pcdh7 | |
| Papss1 | |
| Crlf3 | |
| Tacc1 | |
| Ckap2l | |
| Aes | |
| Klhl9 | |
| Stx6 | |
| Serpinh1 | |
| Eef1d | |
| Lbh | |
| Lgr5 | |

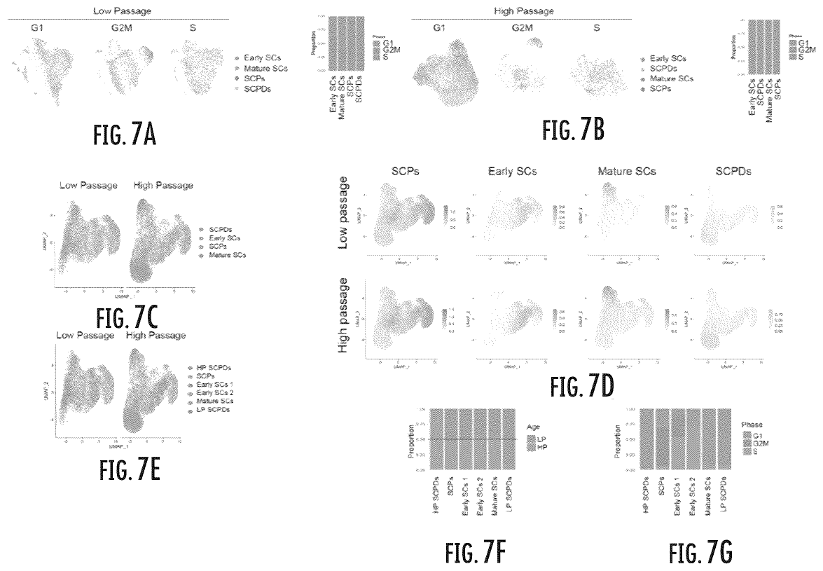

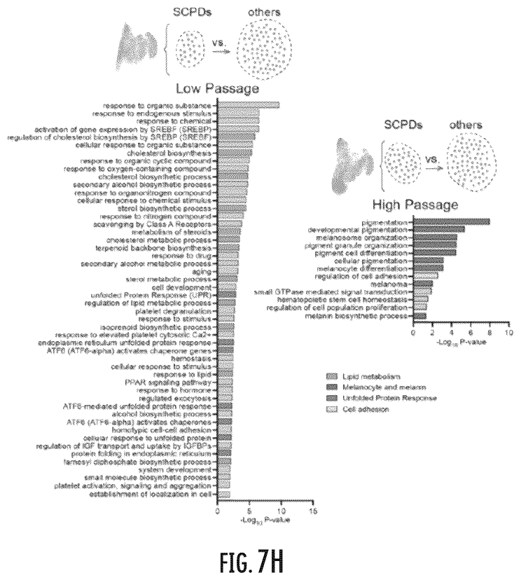

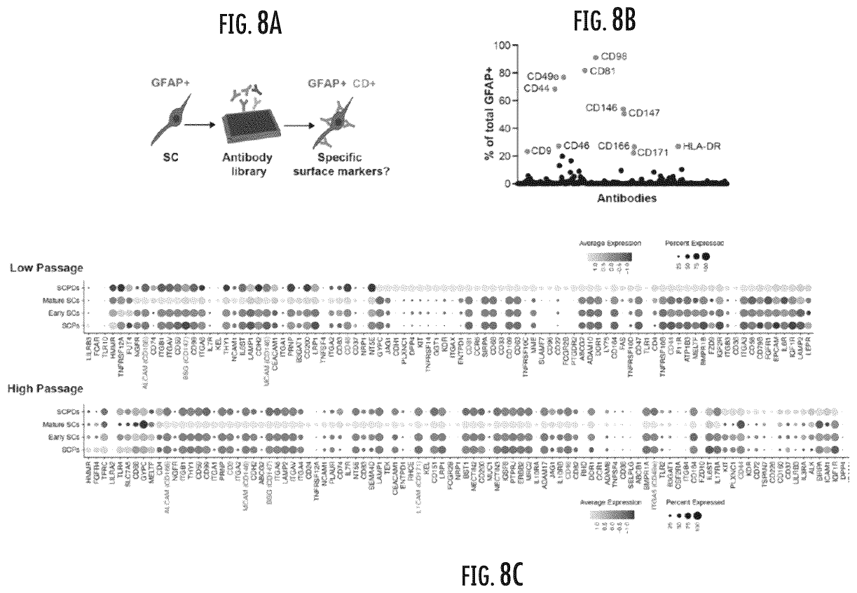

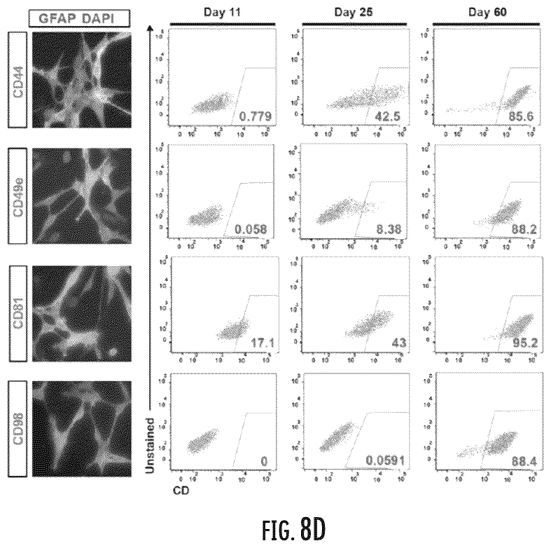

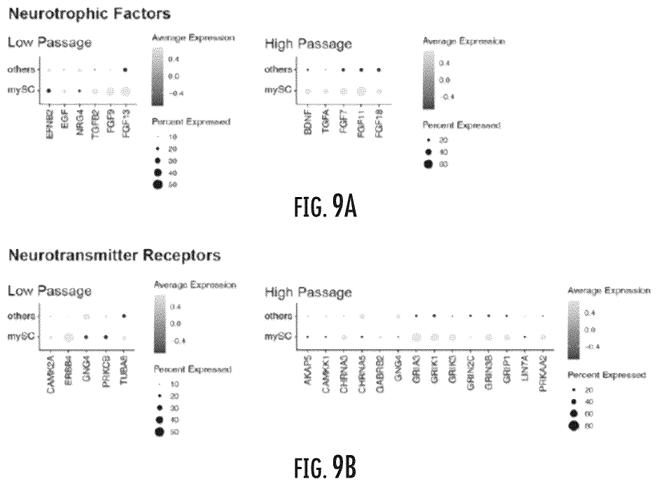

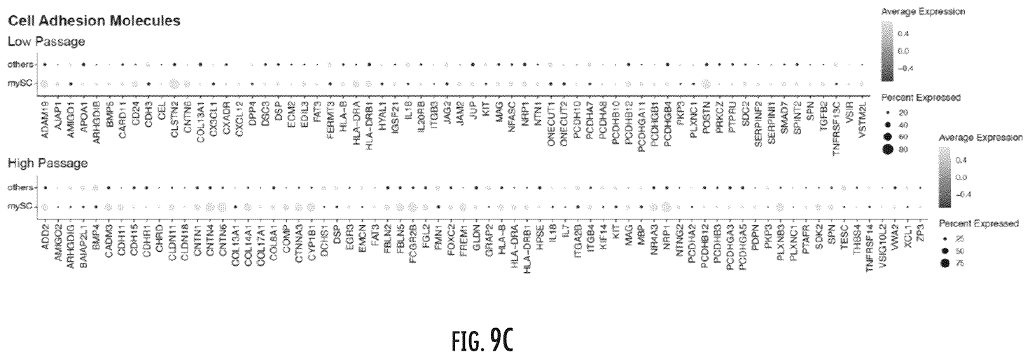

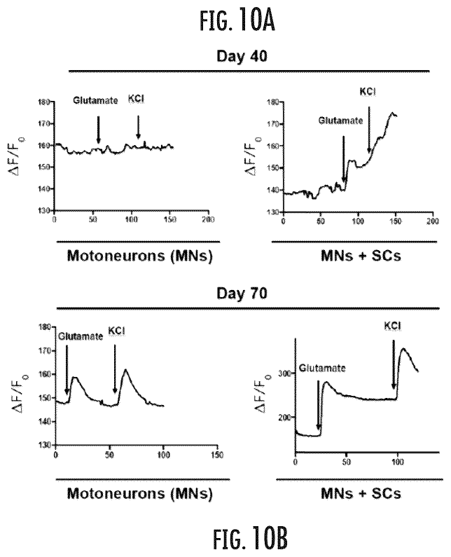

The proliferative capacity of our SC cultures enables their expansion and scalability. To characterize the proliferation potential of our cell types, we determined the proportional distribution of cell cycle phases within individual LP and HP populations (FIG. 7 A and B). As cultures transition from low to high passage, all cell types progressively exit the cell cycle. This is particularly evident in SCPs and early SCs that are predominantly cycling in low passage (FIG. 7 A and B). This is in agreement with the slower proliferation rate of our cultures as they age (not shown).

To assess whether our cultures retain their identity after long-term expansion, we evaluated the lineage relationship between the LP and HP cell types by module scoring the transcriptional signature of LP cell types in HP cell types and vice versa (FIG. 1 H and I). Notably, each cell type signature was most similar to its corresponding cell type in the other dataset (FIG. 1 H and I). The similarity between corresponding LP and HP clusters was further demonstrated when we performed clustering on a merged dataset and obtained the same cell populations (FIG. 7 C and D). Bulk transcriptomics analysis of cultures at different time points demonstrated that hPSC-derived developing precursors were closely related to early NC cells while SC cultures, particularly in higher passages, showed a gene expression pattern closely matching primary adult human SCs (FIG. 1J).

To characterize the diversity in these cultures in higher resolution, we performed further sub-clustering and revealed two early SCs and two SCPDs populations (FIG. S3E). While early SCs 1 and 2 separated solely based on their cell cycle phase distributions (FIG. S3F), SCPDs subclusters were predominantly LP or HP specific (FIG. S3G). To determine if LP and HP SCPDs were functionally distinct, we performed gene ontology (GO) enrichment analysis of their top 250 differentially expressed genes. Interestingly, despite high level expression of canonical melanocytic genes such as MTTF, MLANA and PMEL, LP SCPDs were enriched for myelin production terms, such as cholesterol and lipid metabolism pointing to a dual melanocyte-SC identity (FIG. S2A and FIG. S3H). On the other hand, HP SCPDs displayed enrichment for melanin synthesis and pigmentation (FIG. S3H) indicating that as cultures age SCPDs become more melanocytic.

Strategies for prospective isolation enables the generation of pure and high quality SC populations from heterogeneous cultures. To enable fluorescence-activated cell sorting (FACS) based purification of hPSC-SCs, we screened a library of 242 antibodies for human surface antigens that specifically mark GFAP+ SCs (FIG. S4 A and B). We identified 11 surface antibodies that stained >20% of the GFAP+ SCs of which CD44, CD49e, CD81 and CD98 labeled most of the target population (FIG. S4B). Analysis of surface marker expression in our LP and HP scRNA-seq datasets revealed transcripts that were specifically enriched in each cell type (FIG. S4C). Interestingly, this list included 9 of the 11 surface marker hits identified by the antibody screening. Among these, CD46, CD146, CD147 and CD166 were enriched in mature SCs in both datasets while CD9, CD49e and CD171 were enriched only in HP mature SCs. CD44 was highly expressed in SCPDs in both LP and HP cultures while CD81 enrichment was specific to the LP population (FIG. S4C). Further validations revealed that CD98 was the only marker specifically expressed in SCs but not in NCs or SCPs (FIG. S4D). These populations expressed CD49D, a marker previously shown to label early SOX10+ NC lineages (Fattahi et al., 2016).

Collectively these data demonstrate that our hPSC differentiation system generates scalable and proliferative human SC cultures that can be further enriched using FACS.

hPSC-SCs Promote Neuronal Maturation and Myelination In Vitro and Engraft into Injured Sciatic Nerves in Rats

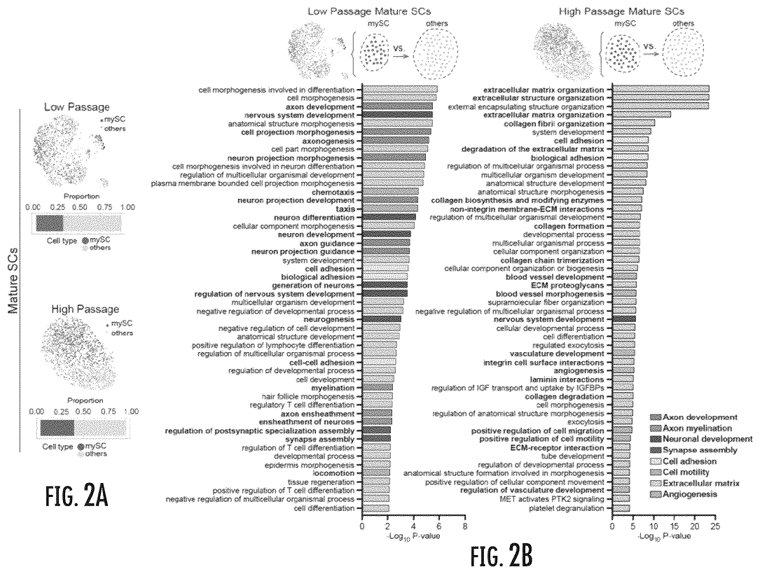

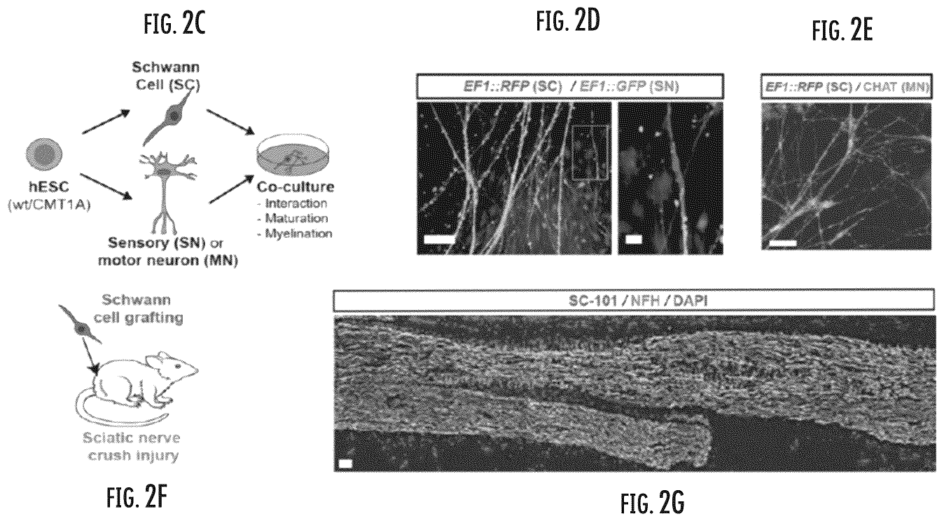

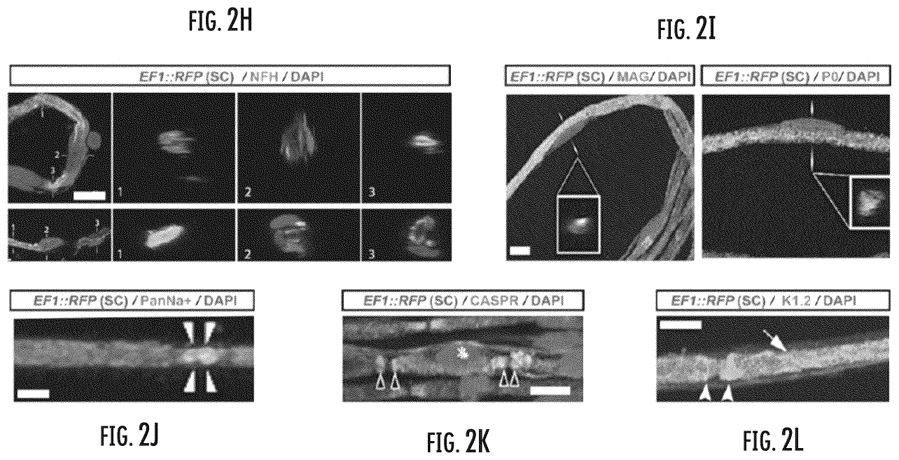

SCs play fundamental roles in maintaining and protecting the structure and function of the peripheral nerves. Myelinating SCs are specialized glial cells that form lipid rich myelin sheaths around the axons and enable fast neuronal signal propagation in the PNS. Since our hPSC-derived mature SCs express genes involved in myelination and lipid metabolism in high levels (FIG. 1 F and G, FIG. S3H), we set out to identify and characterize myelinating SCs (mySCs) in our LP and HP mature SCs. We module scored a curated list of top differentially expressed primary mouse mySCs and canonical myelinating genes (Calder et al., 2015) in our LP and HP mature SCs (FIG. 2A, Table S3). We identified >25% of mature SCs in each dataset as myelinating (FIG. 2A). LP and HP mySCs were specifically enriched for a number of neurotrophic factors and neurotransmitter and postsynaptic transmission genes (FIG. S5 A and B). For example, members of TGF and FGF protein families were highly enriched in mySCs compared to other mature SCs in both datasets (FIG. S5A). BDNF expression was specific to HP mySCs (FIG. S5A). To define the unique functional properties of these cells we performed pathway enrichment analysis using GO BP, KEGG and Reactome gene sets on the significantly upregulated genes in LP and HP mySCs (FIG. 2B). Interestingly, of the top 50 significantly enriched pathways, we identified multiple pathways related to axon development, myelination, neuronal development, synapse assembly, cell adhesion and cell motility. These are well established physiological features of myelinating SCs. HP mySCs upregulated pathways related to extracellular matrix organization, cell adhesion and cell motility among others. This is intriguing given the known contribution of SCs in depositing and organizing ECM components, formation of lamellipodia and cytoplasmic protrusions and forming contact and recognizing axons during radial sorting and myelination. These indicate our LP and HP mySCs are equipped with the molecular programs that enable myelinating SCs to perform their function.

TABLE S3
mySC Markers

MBP
SECISBP2L
POU3F1
FA2H
PTN
CNP
PRX
EMID1
CRYAB
NCMAP
MAL
CLDN19
MPZ
PLLP
PMP22
EGR2
ID2
MAG
BDNF
GDNF
NRG1

Since cell adhesion molecules (CAMs) play an important role in SC association with axons, nerve components and the ECM, and cell adhesion was amongst the most significantly enriched GO terms in both our LP and HP mySCs (FIG. 2B), we sought to determine the specific CAMs that were enriched in mySC populations. We module scored a list of cell adhesion molecules combining cell-cell and cell-matrix gene sets in our LP and HP mature SCs and identified many CAMs that were specifically depleted and enriched in LP and HP mySCs relative to the other mature SCs (FIG. S5C). For example, HLA-DR that was also a hit in our SC antibody screening (FIG. S4B, FIG. S5C) was enriched in both LP and HP mySCs. The proinflammatory interleukin IL18 was depleted while MAG and KIT were enriched in both mySC populations (FIG. S5C). Members of the contactin protein family (CNTN1, 4 and 6) that are axon-associated CAMs and play roles in the formation of axon connections in the developing nervous system were specifically enriched in HP mySCs (FIG. S5C). Similarly, HP mySCs were highly enriched for PLXNB3 which is important for axon guidance and cell migration (FIG. S5C).

To assess the ability of hPSC-derived SCs to functionally interact with neurons, we established co-cultures with hPSC-derived sensory (Chambers et al., 2012) and motor neurons (Calder and Tchieu, 2015) (FIG. 2C). RFP-labeled SCs (day 60) were mixed with GFP-labeled sensory neurons (day 50) and analyzed at 72 hours of co-culture. SCs associated closely with sensory neurons by aligning along their processes (FIG. 2D). Similarly, co-cultures of SCs and hPSC-derived motor neurons (day 25) showed robust interaction along neuronal fibers (FIG. 2E). The protracted process of human cell maturation in hPSC-derived lineages is a major hurdle in the field. Glial cells such as astrocytes have been shown to promote the functional maturation of hPSC-derived CNS neurons (Tang et al., 2013). To assess the impact of SCs on maturation, we performed calcium imaging in hPSC-derived motoneurons at day 40 and 70 of differentiation (15 and 55 days of co-culture). Interestingly, there was a marked increase in the calcium response of stage-matched motoneurons co-cultured with SCs (FIG. S6A). By day 70, the responsiveness to glutamate and KCl stimulations further improved and remained distinct from cultures containing motor neurons only (FIG. S6B). Our findings demonstrate the capability of hPSC-SCs to modulate neuronal function in vitro. In order to determine whether hPSC-SCs are functional in vivo and are capable of producing myelin, we asked whether they could survive and engraft in a rat model of sciatic nerve injury. We depleted endogenous SCs through a mechanical crush of the nerve and injected RFP-labeled hPSC-SCs at the site of injury (FIG. 2F). The transplanted SCs could be readily detected at eight weeks after nerve injection using the human-specific nuclear marker SC101 (FIG. 2G). Transplanted hPSC-SCs were in close contact with the host neurons (FIG. 2H) and expressed the myelin markers MAG and P0 (FIG. 2I). In mature myelinated fibers, sodium channels are localized at nodes of Ranvier: the site of action potential electrogenesis. This area is flanked by a CASPR-expressing domain (juxtaparanodal region) where the axon membrane is in close contact to myelin membrane. Adjacent to the CASPR+ region is an axon membrane domain marked by the expression of potassium channels. Remarkably, we observed appropriate localization of both sodium and potassium channels in axons that were wrapped by RFP-labelled hPSC-SCs (FIG. 2J-L). These studies demonstrate the ability of hPSC-SCs to engraft and produce myelin that is appropriately associated with nerve fibers and the nodes of Ranvier in injured adult peripheral nerves.

These results demonstrate that our hPSC-derived cultures of functional SCs offer a framework for modeling pathologies in which SCs play central roles in disease initiation and progression. For example, a large subset of Charcot-Marie-Tooth (CMT) patients suffer from debilitating myelin defects caused by genetic mutations. Importantly, genes associated with CMT including de-myelinating CMT1, axonal CMT2, and intermediate CMT are expressed by our SC cultures confirming their applicability for modeling CMT pathophysiology in future studies (FIG. S7).

hPSC-Derived SCs Enable Modeling, Mechanistic Understanding and Treating Diabetic Peripheral Neuropathy

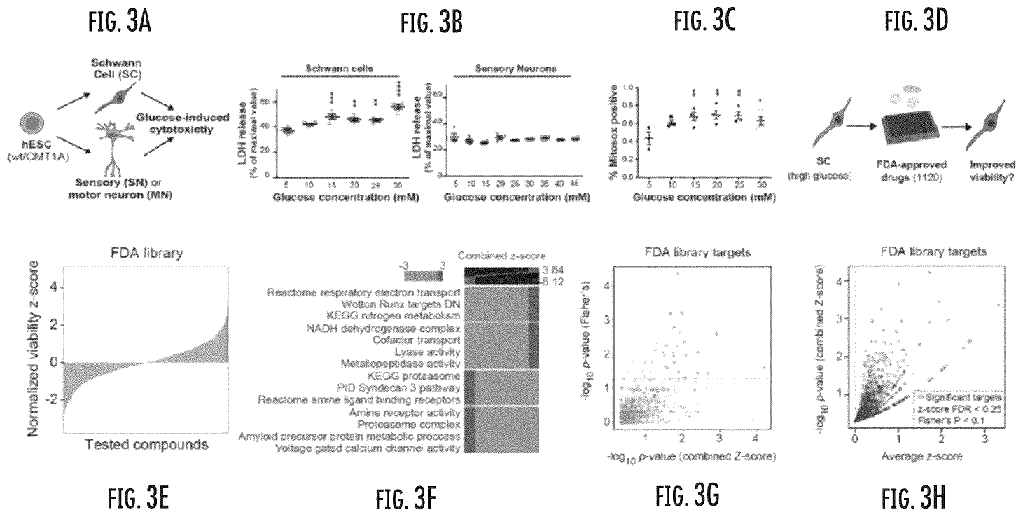

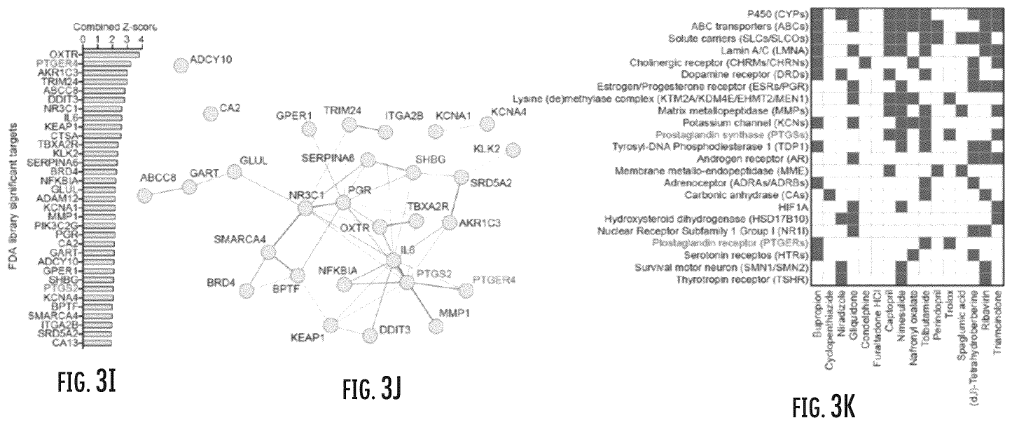

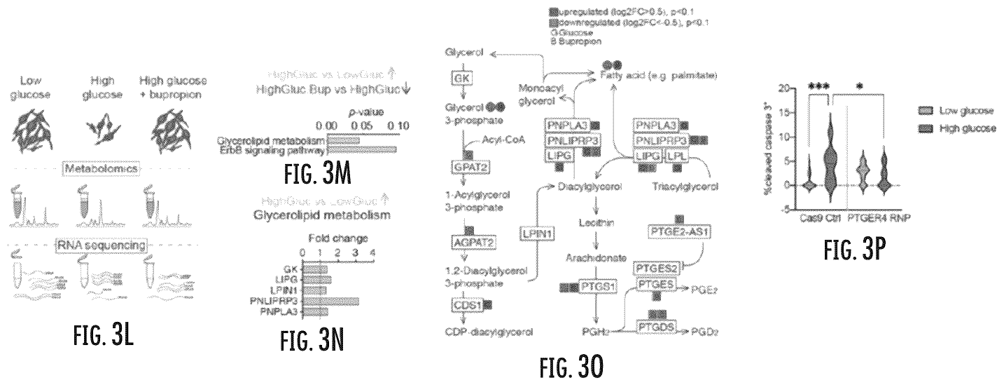

In addition to rare inherited defects such as CMT, SCs are associated with a broad range of other neuropathies. The most prominent form of an acquired neuropathy is diabetic peripheral neuropathy (DPN) which results from the progressive degeneration of peripheral nerves (Simmons and Feldman, 2002). While symptoms arise from neuronal dysfunction, it is unclear whether sensory neuron damage is the primary event in DPN, and there is evidence that SC degeneration and peripheral demyelination may be contributing factors (Eckersley, 2002). As a proof of concept, we set out to leverage our human hPSC differentiation system to model DPN by investigating the effect of high glucose on sensory neurons and SCs (FIG. 3A).

Sensory neurons showed no overt toxicity at glucose levels of up to 45 mM. In contrast, hPSC-derived SC cultures were exquisitely sensitive to even moderately increased glucose levels (FIG. 3B). Treatment with high glucose induced oxidative stress in SC cultures as measured by MitoSOX staining (FIG. 3C).

Given the exquisite glucotoxicity in SCs, strategies that prevent glucose-mediated cellular damage in SCs may represent novel therapeutic opportunities for treating DPN. We established a high-throughput screening (HTS) assay to measure the viability of hPSC-SCs in the presence of 30 mM glucose and the Prestwick library containing 1,120 small molecules of approved drugs (FDA, EMA or other regulatory agencies) (FIG. 3D, FIG. S8A). We identified several hit compounds that significantly increased SC viability under high glucose conditions (FIG. 3E, Table S4). Gaining mechanistic insights on the protective effect of these hits could shed light on the mechanism of glucotoxicity in SCs. Given that the library compounds target many different cellular pathways, we sought to determine the shared pathways among candidate drugs that improved SC viability under the high glucose condition using our previously established analysis approach (Samuel et al., 2020).

TABLE S4

| chemical_name | z.score.t |
|---|---|
| Bupropion hydrochloride | 2.803231907 |
| Cyclopenthiazide | 2.572725364 |
| Niridazole | 2.422433046 |
| Gliquidone | 2.369444837 |
| Condelphine | 2.361110825 |
| Furaltadone hydrochloride | 2.339863469 |
| Captopril | 2.206900457 |
| Nimesulide | 2.110636707 |
| Nafronyl oxalate | 2.094453034 |
| Tolbutamide | 2.089446985 |
| Perindopril | 2.067046408 |
| Trolox | 2.048042926 |
| Spaglumic acid | 2.04596734 |
| (d,l)-Tetrahydroberberine | 2.041477825 |
| Ribavirin | 2.029936986 |
| Triamcinolone | 2.023777849 |
| Phenazopyridine hydrochloride | 1.998973044 |
| Nystatine | 1.991779939 |
| Prilocaine hydrochloride | 1.983928984 |
| Tiletamine hydrochloride | 1.958171411 |
| Dicloxacillin sodium salt | 1.941608221 |
| Lactobionic acid | 1.941608221 |
| Cefotaxime sodium salt | 1.930900501 |
| Dimaprit dihydrochloride | 1.917887741 |
| beta-Belladonnine dichloroethylate | 1.872303174 |
| Norethynodrel | 1.856813543 |
| Sulfapyridine | 1.85320568 |
| Isocorydine (+) | 1.844516974 |
| Lorglumide sodium salt | 1.824052236 |
| Flurbiprofen | 1.824052236 |
| Alfaxalone | 1.82107988 |
| Lansoprazole | 1.808484843 |
| Dinoprost trometamol | 1.788511576 |
| Hesperetin | 1.785196663 |
| Hydrastinine hydrochloride | 1.785196663 |
| Estrone | 1.784197818 |
| Hydrocortisone base | 1.759636688 |
| Mefenamic acid | 1.751378505 |
| Retinoic acid | 1.751378505 |
| Xylazine | 1.749877251 |
| Naproxen | 1.744515391 |
| Clidinium bromide | 1.743166302 |
| Budesonide | 1.740049208 |
| Pyrazinamide | 1.73015172 |
| Cilostazol | 1.73015172 |
| Diflorasone Diacetate | 1.720183935 |
| (−)-Quinpirole hydrochloride | 1.682783502 |
| Betazole hydrochloride | 1.681190012 |
| Methylergometrine maleate | 1.669259859 |
| Ketoconazole | 1.669259859 |
| Phensuximide | 1.66887385 |
| Naringin hydrate | 1.658851699 |
| Tenoxicam | 1.658851699 |
| (−)-MK 801 hydrogen maleate | 1.657320611 |
| Tiabendazole | 1.637804583 |
| Parthenolide | 1.637804583 |
| Azlocillin sodium salt | 1.635022059 |
| Diethylstilbestrol | 1.623741099 |
| Tinidazole | 1.623741099 |
| Muramic acid, N-acetyl | 1.616443316 |
| Flunisolide | 1.612370801 |
| (S)-(−)-Atenolol | 1.605642417 |
| Gelsemine | 1.600909986 |
| Rolitetracycline | 1.600909986 |
| Piracetam | 1.594760002 |
| (−)-Cinchonidine | 1.594760002 |
| Promethazine hydrochloride | 1.577711986 |
| Delsoline | 1.565972356 |
| Methacholine chloride | 1.554137314 |
| Naringenine | 1.539089026 |
| Karakoline | 1.530175919 |
| Furazolidone | 1.530175919 |
| Ethacrynic acid | 1.529514087 |
| Lumicolchicine gamma | 1.527695633 |
| Nifurtimox | 1.521820816 |
| Eburnamonine (−) | 1.516213306 |
| Sulfameter | 1.493486091 |
| Fursultiamine Hydrochloride | 1.482619909 |
| Meloxicam | 1.481221539 |
| Novobiocin sodium salt | 1.481051451 |
| Chenodiol | 1.480752078 |
| Ioxaglic acid | 1.474654036 |
| Dehydrocholic acid | 1.472467093 |
| Hydralazine hydrochloride | 1.472467093 |
| Moroxidine hydrochloride | 1.464135919 |
| Amoxicillin | 1.457428308 |
| Epiandrosterone | 1.457428308 |
| Alclometasone dipropionate | 1.455866985 |
| Ranolazine | 1.442359787 |
| Glipizide | 1.430341288 |
| 2-Aminobenzenesulfonamide | 1.430253029 |
| Metrizamide | 1.421017039 |
| Rauwolscine hydrochloride | 1.383711314 |
| Quercetine dihydrate | 1.371071607 |
| Fluorocurarine chloride | 1.364264529 |
| Aztreonam | 1.360410717 |
| Fusaric acid | 1.345478049 |
| Convolamine hydrochloride | 1.345478049 |
| Clobetasol propionate | 1.323273525 |
| Tetrahydroxy-1,4-quinone monohydrate | 1.323273525 |
| Nafcillin sodium salt monohydrate | 1.323273525 |
| Oxolinic acid | 1.315041141 |
| Harmaline hydrochloride dihydrate | 1.306283484 |
| Nandrolone | 1.305878261 |
| Dantrolene sodium salt | 1.305807694 |
| Ampicillin trihydrate | 1.28717778 |
| Prednisolone | 1.28717778 |
| Picrotoxinin | 1.281184975 |
| (+) -Levobunolol hydrochloride | 1.281184975 |
| Dextromethorphan hydrobromide monohydrate | 1.279602819 |
| Iopanoic acid | 1.278903975 |
| Chloramphenicol | 1.277780439 |
| Fludrocortisone acetate | 1.277780439 |
| Isometheptene mucate | 1.269810256 |
| Tolazoline hydrochloride | 1.268327588 |
| Bezafibrate | 1.266093371 |
| Carteolol hydrochloride | 1.266093371 |
| Metronidazole | 1.258818792 |
| Metanephrine hydrochloride DL | 1.258818792 |
| Glimepiride | 1.252496077 |
| Gabazine | 1.252469473 |
| Cinchonine | 1.252469473 |
| Fenbufen | 1.24925361 |
| Nitrocaramiphen hydrochloride | 1.242216755 |
| Isoxicam | 1.239631606 |
| Bromperidol | 1.238729847 |
| Halcinonide | 1.237958126 |
| Bergenin monohydrate | 1.237958126 |
| Methylatropine nitrate | 1.237958126 |
| Imidurea | 1.223556889 |
| Metoprolol-(+, −) (+)-tartrate salt | 1.223289158 |
| Leucomisine | 1.214146527 |
| Metergoline | 1.210898304 |
| Propafenone hydrochloride | 1.210898304 |
| Ethamivan | 1.210898304 |
| Demecarium bromide | 1.210898304 |
| Riboflavine | 1.210898304 |
| Flavoxate hydrochloride | 1.21042028 |
| Aminophylline | 1.208487666 |
| Droperidol | 1.196803845 |
| Betonicine | 1.193552149 |
| Epirizole | 1.190653953 |
| Famotidine | 1.190653953 |
| Bumetanide | 1.180681859 |
| Hippeastrine hydrobromide | 1.178481117 |
| Estropipate | 1.178481117 |
| Napelline | 1.178481117 |
| Amiprilose hydrochloride | 1.178481117 |
| Metyrapone | 1.178481117 |
| Azapropazone | 1.175959846 |
| Pentylenetetrazole | 1.170649906 |
| Chlorothiazide | 1.170649906 |
| Hydrocotarnine hydrobromide | 1.168251253 |
| Flumethasone | 1.163273092 |
| Carbenoxolone disodium salt | 1.163273092 |
| Bacitracin | 1.163273092 |
| Doxycycline hyclate | 1.147926609 |
| Roxatidine Acetate HCl | 1.139205473 |
| Pilocarpine nitrate | 1.139205473 |
| Rifampicin | 1.139205473 |
| Triflusal | 1.139205473 |
| Sulfamethizole | 1.132440222 |
| Ethambutol dihydrochloride | 1.129915056 |
| Methoxy-8-psoralen | 1.12449458 |
| Methionine sulfoximine (L) | 1.12449458 |
| Ungerine nitrate | 1.116812507 |
| Sulfaquinoxaline sodium salt | 1.116812507 |
| Flunixin meglumine | 1.116812507 |
| Thiocolchicoside | 1.109656755 |
| Spectinomycin dihydrochloride | 1.101042061 |
| Methylhydantoin-5-(L) | 1.101042061 |
| Adrenosterone | 1.101042061 |
| Thiamphenicol | 1.098714923 |
| Benfluorex hydrochloride | 1.094690825 |
| Corynanthine hydrochloride | 1.094690825 |
| Canavanine sulfate monohydrate (L, +) | 1.094690825 |
| Baclofen (R,S) | 1.088189105 |
| Tranexamic acid | 1.088189105 |
| Metaraminol bitartrate | 1.088189105 |
| Cephalexin monohydrate | 1.079595643 |
| Clebopride maleate | 1.079595643 |
| Trimethadione | 1.079595643 |
| Diloxanide furoate | 1.069067523 |
| Troleandomycin | 1.066946472 |
| Methoxamine hydrochloride | 1.064370086 |
| Chlorhexidine | 1.0562289 |
| Clonidine hydrochloride | 1.0562289 |
| Helveticoside | 1.052860785 |
| Esculin Hydrate | 1.052860785 |
| Clozapine | 1.049013063 |
| Adenosine 5′-monophosphate monohydrate | 1.049013063 |
| Meclocycline sulfosalicylate | 1.049013063 |
| Pentoxifylline | 1.045446666 |
| Indomethacin | 1.045446666 |
| Cholecalciferol | 1.033523516 |
| Ginkgolide A | 1.033523516 |
| Dapsone | 1.023686792 |
| Metoclopramide monohydrochloride | 1.023686792 |
| S-(+)-ibuprofen | 1.020002055 |
| Neostigmine bromide | 1.017900423 |
| Berberine chloride | 1.017900423 |
| Gramine | 1.017900423 |
| N6-methyladenosine | 1.002142804 |
| Etifenin | 1.002142804 |
| Harmane hydrochloride | 1.002142804 |
| Diphenidol hydrochloride | 0.990553376 |
| Myosmine | 0.986541782 |
| Caffeic acid | 0.986541782 |
| Resveratrol | 0.970220294 |
| Ethynylestradiol 3-methyl ether | 0.969583325 |
| Clonixin Lysinate | 0.960333442 |
| Cortisone | 0.956820088 |
| Cinoxacin | 0.952471315 |
| Propantheline bromide | 0.952471315 |
| Hymecromone | 0.952471315 |
| Dihydroergotoxine mesylate | 0.937749113 |
| Suxibuzone | 0.935204839 |
| Equilin | 0.935204839 |
| Etofylline | 0.922479377 |
| Trigonelline | 0.921305871 |
| Solasodine | 0.917783064 |
| Leflunomide | 0.917783064 |
| Iocetamic acid | 0.917783064 |
| THIP Hydrochloride | 0.916219127 |
| D-cycloserine | 0.916219127 |
| Sulfathiazole | 0.910896354 |
| Phenformin hydrochloride | 0.910896354 |
| Kawain | 0.910896354 |
| Guanfacine hydrochloride | 0.904723313 |
| Fenoterol hydrobromide | 0.899244921 |
| Tocainide hydrochloride | 0.893782787 |
| Chlorogenic acid | 0.888000863 |
| Methotrexate | 0.887524892 |
| Aminohippuric acid | 0.882470711 |
| Thioperamide maleate | 0.882470711 |
| Idoxuridine | 0.875736105 |
| Moxisylyte hydrochoride | 0.875736105 |
| Betamethasone | 0.871138028 |
| Carcinine | 0.871138028 |
| Ricinine | 0.864578919 |
| Acetazolamide | 0.863878412 |
| Metformin hydrochloride | 0.863878412 |
| Phenelzine sulfate | 0.851951692 |
| Articaine hydrochloride | 0.848145213 |
| Piperacillin sodium salt | 0.846529413 |
| Butamben | 0.846529413 |
| Ketorolac tromethamine | 0.846529413 |
| Naloxone hydrochloride | 0.839955841 |
| Ambroxol hydrochloride | 0.836989643 |
| Cyanocobalamin | 0.836989643 |
| (−) -Levobunolol hydrochloride | 0.828321859 |
| Mianserine hydrochloride | 0.827890783 |
| Carbamazepine | 0.81575646 |
| Dydrogesterone | 0.809956043 |
| Tetrahydrozoline hydrochloride | 0.809956043 |
| Xamoterol hemifumarate | 0.809956043 |
| Allantoin | 0.803552845 |
| Phenacetin | 0.802275972 |
| Nifuroxazide | 0.802275972 |
| Galanthamine hydrobromide | 0.802275972 |
| Sulfadoxine | 0.801482332 |
| Balsalazide Sodium | 0.791431885 |
| Hexamethonium dibromide dihydrate | 0.791279933 |
| Edrophonium chloride | 0.791279933 |
| Khellin | 0.791279933 |
| Clopamide | 0.784706909 |
| Chloroquine diphosphate | 0.784706909 |
| Pargyline hydrochloride | 0.778937749 |
| Clemizole hydrochloride | 0.778937749 |
| Bucladesine sodium salt | 0.772749439 |
| Nitrofural | 0.76699645 |
| Imipenem | 0.76699645 |
| Seneciphylline | 0.76699645 |
| Boldine | 0.76699645 |
| Naphazoline hydrochloride | 0.766526342 |
| Labetalol hydrochloride | 0.766526342 |
| Methylprednisolone, 6-alpha | 0.766526342 |
| Naltrexone hydrochloride dihydrate | 0.754045796 |
| Iodixanol | 0.75390891 |
| Brinzolamide | 0.74914481 |
| Mimosine | 0.74914481 |
| Heptaminol hydrochloride | 0.741496223 |
| Diprophylline | 0.741496223 |
| Nadolol | 0.734910658 |
| Dehydroisoandosterone 3-acetate | 0.734910658 |
| Amphotericin B | 0.731152332 |
| Mebhydroline 1,5-naphtalenedisulfonate | 0.731152332 |
| Phenindione | 0.731152332 |
| Folic acid | 0.731152332 |
| Nomegestrol acetate | 0.729562887 |
| Estriol | 0.729562887 |
| Diflunisal | 0.716190595 |
| Dropropizine (R,S) | 0.716190595 |
| Avermectin B1 | 0.715755207 |
| Phenethicillin potassium salt | 0.715755207 |
| Spiramycin | 0.715755207 |
| Guanadrel sulfate | 0.715755207 |
| Pyrithyldione | 0.715755207 |
| Proparacaine hydrochloride | 0.715755207 |
| Hydroxytacrine maleate (R,S) | 0.713019489 |
| Myricetin | 0.713019489 |
| Molsidomine | 0.713019489 |
| Ticlopidine hydrochloride | 0.703434925 |
| Cefoxitin sodium salt | 0.696443257 |
| L(−)-vesamicol hydrochloride | 0.696443257 |
| Diltiazem hydrochloride | 0.690611001 |
| Iproniazide phosphate | 0.690611001 |
| Guanethidine sulfate | 0.690611001 |
| (S)-propranolol hydrochloride | 0.680341572 |
| Yohimbine hydrochloride | 0.676335321 |
| Mexiletine hydrochloride | 0.664759542 |
| Debrisoquin sulfate | 0.65778567 |
| Chicago sky blue 6B | 0.65778567 |
| (+, −)-Octopamine hydrochloride | 0.657353562 |
| Trapidil | 0.65535324 |
| Dubinidine | 0.65535324 |
| Panthenol (D) | 0.651732679 |
| Vinpocetine | 0.651732679 |
| Isradipine | 0.642765711 |
| Benzonatate | 0.638638909 |
| Ciprofloxacin hydrochloride | 0.638638909 |
| Sulmazole | 0.637578159 |
| Cloxacillin sodium salt | 0.625478662 |
| Fusidic acid sodium salt | 0.620276608 |
| Quipazine dimaleate salt | 0.620276608 |
| Nitrendipine | 0.617650952 |
| Thiostrepton | 0.601319845 |
| Nicorandil | 0.598960692 |
| Isoetharine mesylate salt | 0.597573634 |
| Iodipamide | 0.597573634 |
| Sulfadiazine | 0.585604048 |
| Pyrimethamine | 0.585604048 |
| Acetohexamide | 0.585604048 |
| Flutamide | 0.585604048 |
| Midodrine hydrochloride | 0.585604048 |
| Niflumic acid | 0.585604048 |
| Moclobemide | 0.582230318 |
| Vancomycin hydrochloride | 0.582230318 |
| Palmatine chloride | 0.582230318 |
| Conessine | 0.582230318 |
| Chlorzoxazone | 0.572183087 |
| Pyrantel tartrate | 0.572183087 |
| Ceftazidime pentahydrate | 0.563009798 |
| Clorsulon | 0.563009798 |
| Diclofenac sodium | 0.563009798 |
| Thiorphan | 0.563009798 |
| Procaine hydrochloride | 0.558698463 |
| Deferoxamine mesylate | 0.556976555 |
| Cromolyn disodium salt | 0.556976555 |
| Monobenzone | 0.556976555 |
| Chlorpheniramine maleate | 0.545150868 |
| Tiapride hydrochloride | 0.545150868 |
| Telenzepine dihydrochloride | 0.545150868 |
| Alizapride HCl | 0.543660245 |
| Cyclizine hydrochloride | 0.543660245 |
| Bicuculline (+) | 0.543660245 |
| Fillalbin | 0.536461323 |
| Nabumetone | 0.536461323 |
| Florfenicol | 0.536461323 |
| Quinic acid | 0.52675638 |
| Azacyclonol | 0.517869789 |
| Guanabenz acetate | 0.517869789 |
| Fipexide hydrochloride | 0.517869789 |
| Bephenium hydroxynaphthoate | 0.515805051 |
| Gabexate mesilate | 0.513574368 |
| Ritodrine hydrochloride | 0.504582842 |
| Dequalinium dichloride | 0.504582842 |
| Cefadroxil | 0.504582842 |
| Cefixime | 0.504582842 |
| Monocrotaline | 0.504582842 |
| Atracurium besylate | 0.504137931 |
| Amyleine hydrochloride | 0.504137931 |
| Isoniazid | 0.504137931 |
| Salbutamol | 0.504137931 |
| Graveoline | 0.495010616 |
| Ergocryptine-alpha | 0.495010616 |
| Fosfosal | 0.495010616 |
| Triamterene | 0.490346358 |
| Bisacodyl | 0.48485989 |
| Calycanthine | 0.48485989 |
| Flurandrenolide | 0.474081144 |
| Norcyclobenzaprine | 0.465017711 |
| Sulfaphenazole | 0.462587861 |
| Diazoxide | 0.462587861 |
| Nadide | 0.453020022 |
| Piromidic acid | 0.453020022 |
| Sulfamonomethoxine | 0.453020022 |
| Ethaverine hydrochloride | 0.453020022 |
| Azathioprine | 0.448622956 |
| Cyproheptadine hydrochloride | 0.448622956 |
| Dexamethasone acetate | 0.448622956 |
| Ranitidine hydrochloride | 0.448622956 |
| Tiratricol, 3,3′,5-triiodothyroacetic acid | 0.448622956 |
| Gemfibrozil | 0.448622956 |
| Calciferol | 0.445059264 |
| Metaproterenol sulfate, orciprenaline sulfate | 0.445059264 |
| Cimetidine | 0.434602374 |
| Isotretinoin | 0.434602374 |
| Trazodone hydrochloride | 0.434602374 |
| Demeclocycline hydrochloride | 0.431830899 |
| Pipenzolate bromide | 0.431830899 |
| Pipemidic acid | 0.431830899 |
| Nisoxetine hydrochloride | 0.431830899 |
| Carisoprodol | 0.424987719 |
| Ampyrone | 0.420527252 |
| Disopyramide | 0.420527252 |
| Flecainide acetate | 0.410517684 |
| Moxalactam disodium salt | 0.410517684 |
| Niacin | 0.410517684 |
| Urapidil hydrochloride | 0.410517684 |
| Zimelidine dihydrochloride monohydrate | 0.406398773 |
| Ethosuximide | 0.406398773 |
| Levamisole hydrochloride | 0.406398773 |
| Clenbuterol hydrochloride | 0.404806459 |
| Salsolinol hydrobromide | 0.404806459 |
| Acetylsalicylsalicylic acid | 0.392218174 |
| Norfloxacin | 0.392218174 |
| Sulfinpyrazone | 0.392218174 |
| Amethopterin (R,S) | 0.384519071 |
| Iobenguane sulfate | 0.384519071 |
| Tolnaftate | 0.37798674 |
| Diethylcarbamazine citrate | 0.37798674 |
| Isopropamide iodide | 0.36753593 |
| Alfadolone acetate | 0.365079292 |
| Buflomedil hydrochloride | 0.364129356 |
| Metolazone | 0.363705808 |
| Ebselen | 0.34587652 |
| Cisapride | 0.343641319 |
| Alprostadil | 0.337454584 |
| Roxithromycin | 0.324111274 |
| Bemegride | 0.324111274 |
| Milrinone | 0.323578372 |
| Bethanechol chloride | 0.323578372 |
| Bacampicillin hydrochloride | 0.323059174 |
| Dirithromycin | 0.323059174 |
| Acebutolol hydrochloride | 0.320580115 |
| Aminopurine, 6-benzyl | 0.320580115 |
| Mecamylamine hydrochloride | 0.309661385 |
| Carbetapentane citrate | 0.302387333 |
| Ethionamide | 0.302387333 |
| Isoquinoline, 6,7-dimethoxy-1-methyl- | 0.302387333 |
| 1,2,3,4-tetrahydro, hydrochloride | |
| Nitrarine dihydrochloride | 0.302245397 |
| Sulfamerazine | 0.302245397 |
| Sulfamethazine sodium salt | 0.302245397 |
| Benzthiazide | 0.302245397 |
| Acyclovir | 0.291608857 |
| Ethisterone | 0.291608857 |
| Mesoridazine besylate | 0.281630407 |
| Fluocinonide | 0.280284343 |
| Catharanthine | 0.262475795 |
| Terazosin hydrochloride | 0.258233802 |
| Primidone | 0.258233802 |
| Minoxidil | 0.247852789 |
| Trimethoprim | 0.247852789 |
| Norethindrone | 0.247852789 |
| Triprolidine hydrochloride | 0.247852789 |
| Hesperidin | 0.23988069 |
| Torsemide | 0.239515184 |
| Mevalonic-D, L acid lactone | 0.236099698 |
| Trimipramine maleate salt | 0.236099698 |
| Suprofen | 0.236099698 |
| Catechin-(+, −) hydrate | 0.236099698 |
| N-Acetyl-L-leucine | 0.236099698 |
| Nicardipine hydrochloride | 0.218898047 |
| Biotin | 0.218898047 |
| Trimetazidine dihydrochloride | 0.218898047 |
| Sulfamethoxypyridazine | 0.213888171 |
| Ethopropazine hydrochloride | 0.213888171 |
| Chrysin | 0.213888171 |
| 2-Chloropyrazine | 0.211223227 |
| Pindolol | 0.203779052 |
| Pentolinium bitartrate | 0.203779052 |
| Chlorpropamide | 0.197850599 |
| Lysergol | 0.197850599 |
| Mometasone furoate | 0.197850599 |
| Acetopromazine maleate salt | 0.197850599 |
| Cefmetazole sodium salt | 0.191605571 |
| Levonordefrin | 0.191605571 |
| Indoprofen | 0.191605571 |
| Mefexamide hydrochloride | 0.189025687 |
| Apramycin | 0.182817217 |
| Progesterone | 0.176743832 |
| Tolazamide | 0.176743832 |
| Ajmaline | 0.176743832 |
| Hydrochlorothiazide | 0.174244413 |
| Fenofibrate | 0.174244413 |
| Deltaline | 0.169258443 |
| Epitiostanol | 0.168575678 |
| Sulpiride | 0.159437177 |
| Doxepin hydrochloride | 0.159437177 |
| Sulfisoxazole | 0.15558338 |
| Clorgyline hydrochloride | 0.15558338 |
| Benperidol | 0.15558338 |
| Papaverine hydrochloride | 0.15558338 |
| Beta-sistosterol | 0.15431077 |
| Fluticasone propionate | 0.15431077 |
| Pancuronium bromide | 0.15431077 |
| Etanidazole | 0.146853507 |
| Hexestrol | 0.146853507 |
| Sulfadimethoxine | 0.146853507 |
| Ifosfamide | 0.146853507 |
| Ornidazole | 0.144605952 |
| Lincomycin hydrochloride | 0.144605952 |
| Methapyrilene hydrochloride | 0.134375011 |
| Probucol | 0.134375011 |
| Omeprazole | 0.134375011 |
| Sulfasalazine | 0.134375011 |
| Nalbuphine hydrochloride | 0.129752746 |
| Pergolide mesylate | 0.129752746 |
| Folinic acid calcium salt | 0.124397647 |
| Medrysone | 0.124397647 |
| Fenoprofen calcium salt dihydrate | 0.124397647 |
| Nilutamide | 0.124397647 |
| Danazol | 0.114879588 |
| Glutethimide, para-amino | 0.114879588 |
| Sparteine (−) | 0.113124613 |
| Hydroquinine hydrobromide hydrate | 0.113124613 |
| Vidarabine | 0.101897895 |
| (+)-Isoproterenol (+)-bitartrate salt | 0.101897895 |
| Spiperone | 0.099988541 |
| Tranylcypromine hydrochloride | 0.085081681 |
| Aceclofenac | 0.085081681 |
| Dimenhydrinate | 0.085081681 |
| Vincamine | 0.085081681 |
| Lisinopril | 0.085081681 |
| Moricizine hydrochloride | 0.082698584 |
| Pivmecillinam hydrochloride | 0.082698584 |
| Oxaprozin | 0.082698584 |
| Carbinoxamine maleate salt | 0.079361407 |
| Scopolamine hydrochloride | 0.079361407 |
| Kanamycin A sulfate | 0.070521816 |
| Amiloride hydrochloride dihydrate | 0.070161113 |
| Pyrilamine maleate | 0.070161113 |
| Eucatropine hydrochloride | 0.056795449 |
| Benoxinate hydrochloride | 0.055228952 |
| Diphemanil methylsulfate | 0.055228952 |
| Spironolactone | 0.055228952 |
| Canrenoic acid potassium salt | 0.055228952 |
| Remoxipride Hydrochloride | 0.053953912 |
| Tiaprofenic acid | 0.049181677 |
| Biperiden hydrochloride | 0.049181677 |
| Dimethisoquin hydrochloride | 0.049181677 |
| Antipyrine, 4-hydroxy | 0.040287335 |
| Fluvoxamine maleate | 0.039567727 |
| Mepenzolate bromide | 0.034207368 |
| Streptozotocin | 0.034207368 |
| Capsaicin | 0.034207368 |
| Phenoxybenzamine hydrochloride | 0.034207368 |
| Thioguanosine | 0.027823998 |
| Chlortetracycline hydrochloride | 0.025338404 |
| Sulfamethoxazole | 0.025338404 |
| Indapamide | 0.025338404 |
| Risperidone | 0.025174859 |
| N-Acetyl-DL-homocysteine Thiolactone | 0.01160458 |
| Evoxine | 0.01160458 |
| Norgestrel-(−)-D | 0.01160458 |
| Meptazinol hydrochloride | 0.010777233 |
| Methyldopa (L, −) | 0.006455057 |
| Domperidone | 0.006455057 |
| Aspartic acid, N-acetyl (R,S) | 0.006455057 |
| Melatonin | 0.006455057 |
| Felodipine | 0.006455057 |
| Rescinnamin | 0.006455057 |
| Nomifensine maleate | −0.004572767 |
| Antazoline hydrochloride | −0.004572767 |
| Rolipram | −0.011005454 |
| Procarbazine hydrochloride | −0.018024574 |
| LidocaÂ<ne hydrochloride | −0.019530679 |
| Fenspiride hydrochloride | −0.019530679 |
| Acacetin | −0.033615255 |
| Cefsulodin sodium salt | −0.033615255 |
| Trichlormethiazide | −0.033615255 |
| Dimethadione | −0.033615255 |
| Idazoxan hydrochloride | −0.033615255 |
| Nitrofurantoin | −0.034487255 |
| Pinacidil | −0.034487255 |
| Gliclazide | −0.036291367 |
| (R)-Propranolol hydrochloride | −0.046822234 |
| Trichlorfon | −0.049440327 |
| Picotamide monohydrate | −0.049440327 |
| Vitexin | −0.05621734 |
| Methimazole | −0.05621734 |
| Tolmetin sodium salt dihydrate | −0.05621734 |
| Flucytosine | −0.05621734 |
| Morantel tartrate | −0.064387737 |
| Procyclidine hydrochloride | −0.078804256 |
| Felbinac | −0.078804256 |
| Nizatidine | −0.078804256 |
| Hemicholinium bromide | −0.079007077 |
| Terconazole | −0.079007077 |
| Iohexol | −0.079007077 |
| Pirenperone | −0.079007077 |
| Trimethylcolchicinic acid | −0.079007077 |
| Pirenzepine dihydrochloride | −0.07932733 |
| Didanosine | −0.07932733 |
| Benzathine benzylpenicillin | −0.089977188 |
| Pheniramine maleate | −0.094256965 |
| Amoxapine | −0.094256965 |
| Erythromycin | −0.094256965 |
| Chlorphensin carbamate | −0.094256965 |
| Oxytetracycline dihydrate | −0.094256965 |
| Bisoprolol fumarate | −0.100337702 |
| Piperine | −0.100337702 |
| 3-alpha-Hydroxy-5-beta-androstan-17-one | −0.101368588 |
| Sulfachloropyridazine | −0.101368588 |
| (−)-Isoproterenol hydrochloride | −0.104343432 |
| Hydroflumethiazide | −0.109174509 |
| Proguanil hydrochloride | −0.118697142 |
| Methoxy-6-harmalan | −0.12164188 |
| Benfotiamine | −0.123902991 |
| Anabasine | −0.123902991 |
| Althiazide | −0.123902991 |
| Carbachol | −0.123902991 |
| Tropicamide | −0.124077851 |
| Phentolamine hydrochloride | −0.124077851 |
| Moxonidine | −0.133036442 |
| Cotinine (−) | −0.138964891 |
| Etodolac | −0.138964891 |
| Quinapril HCl | −0.146400209 |
| Rifabutin | −0.147359473 |
| Doxylamine succinate | −0.153833556 |
| Dizocilpine maleate | −0.153833556 |
| Gallamine triethiodide | −0.153833556 |
| Cyclobenzaprine hydrochloride | −0.164146433 |
| Cytisine (−) | −0.164146433 |
| Isoxsuprine hydrochloride | −0.168681792 |
| Molindone hydrochloride | −0.175949382 |
| Racecadotril | −0.185334805 |
| Etomidate | −0.190212645 |
| Trioxsalen | −0.191254613 |
| Glycopyrrolate | −0.191254613 |
| Drofenine hydrochloride | −0.191254613 |
| Scopolamin-N-oxide hydrobromide | −0.1983089 |
| Yohimbinic acid monohydrate | −0.206472749 |
| Antipyrine | −0.213083806 |
| Nicergoline | −0.213083806 |
| Sulconazole nitrate | −0.213597904 |
| Beclomethasone dipropionate | −0.213597904 |
| Pivampicillin | −0.218666934 |
| Carbimazole | −0.227554557 |
| TetracaÂ<ne hydrochloride | −0.227554557 |
| Homatropine hydrobromide (R,S) | −0.227830353 |
| Amidopyrine | −0.227830353 |
| Loxapine succinate | −0.227830353 |
| Pizotifen malate | −0.2328545 |
| (+, −)-Synephrine | −0.2328545 |
| Viomycin sulfate | −0.2328545 |
| Isopyrin hydrochloride | −0.235876251 |
| Mephentermine hemisulfate | −0.235876251 |
| Butylparaben | −0.235876251 |
| Dioxybenzone | −0.235876251 |
| Benserazide hydrochloride | −0.235876251 |
| Sulfaguanidine | −0.242546642 |
| Sulindac | −0.242546642 |
| Acenocoumarol | −0.242546642 |
| Tetracycline hydrochloride | −0.242546642 |
| Nifenazone | −0.242546642 |
| Deptropine citrate | −0.247013422 |
| Corticosterone | −0.248574655 |
| Aconitine | −0.248574655 |
| Epivincamine | −0.248574655 |
| Solanine alpha | −0.248574655 |
| Alprenolol hydrochloride | −0.257230804 |
| Arbutin | −0.261142047 |
| Ciprofibrate | −0.261142047 |
| Trimethobenzamide hydrochloride | −0.271881016 |
| Foliosidine | −0.280212181 |
| Clioquinol | −0.280212181 |
| Sulfacetamide sodic hydrate | −0.286495487 |
| Dicyclomine hydrochloride | −0.286495487 |
| Procainamide hydrochloride | −0.290408202 |
| Hycanthone | −0.290408202 |
| Apigenin | −0.290408202 |
| Propylthiouracil | −0.290408202 |
| Tomatidine | −0.290408202 |
| Lobeline alpha (−) hydrochoride | −0.290408202 |
| Hydroxyzine dihydrochloride | −0.301072475 |
| Heliotrine | −0.3022573 |
| Guaifenesin | −0.3022573 |
| Oxalamine citrate salt | −0.3022573 |
| Butacaine | −0.3022573 |
| Acetaminophen | −0.3022573 |
| Proxyphylline | −0.3022573 |
| Saquinavir mesylate | −0.303330104 |
| Practolol | −0.311211307 |
| Cetirizine dihydrochloride | −0.311211307 |
| Isoflupredone acetate | −0.315610273 |
| Meticrane | −0.315610273 |
| Bupivacaine hydrochloride | −0.315610273 |
| Ondansetron Hydrochloride | −0.317321685 |
| Methantheline bromide | −0.317321685 |
| Dicumarol | −0.324212571 |
| Nifedipine | −0.330107226 |
| Mebeverine hydrochloride | −0.330107226 |
| Pirlindole mesylate | −0.331275223 |
| Meropenem | −0.331275223 |
| Sisomicin sulfate | −0.331932029 |
| Probenecid | −0.331932029 |
| Tetramisole hydrochloride | −0.331932029 |
| Dihydroergocristine mesylate | −0.331932029 |
| Lobelanidine hydrochloride | −0.331932029 |
| Laudanosine (R,S) | −0.331932029 |
| Protoveratrine A | −0.331932029 |
| Riluzole hydrochloride | −0.344561717 |
| Gibberellic acid | −0.345189284 |
| Pentetic acid | −0.346072315 |
| Eserine sulfate, physostigmine sulfate | −0.352565649 |
| Retrorsine | −0.352565649 |
| Dihydrostreptomycin sulfate | −0.358972182 |
| Methazolamide | −0.367831097 |
| Protriptyline hydrochloride | −0.367831097 |
| (S)-(−)-Cycloserine | −0.372893424 |
| Dobutamine hydrochloride | −0.373107643 |
| Noscapine | −0.373107643 |
| Clindamycin hydrochloride | −0.373337097 |
| Nefopam hydrochloride | −0.373337097 |
| 6-Hydroxytropinone | −0.386680835 |
| Cefalonium | −0.386680835 |
| Bromopride | −0.389483724 |
| (R) -Naproxen sodium salt | −0.389483724 |
| Ethynodiol diacetate | −0.389483724 |
| Bretylium tosylate | −0.389483724 |
| Mitoxantrone dihydrochloride | −0.393553681 |
| Oxantel pamoate | −0.393553681 |
| Tobramycin | −0.393553681 |
| Mafenide hydrochloride | −0.401924448 |
| Epicatechin-(−) | −0.411025261 |
| Austricine hydrate | −0.411025261 |
| Trimeprazine tartrate | −0.411025261 |
| Propofol | −0.411025261 |
| Pralidoxime chloride | −0.411025261 |
| Tremorine dihydrochloride | −0.413899641 |
| SR-95639A | −0.413899641 |
| Colistin sulfate | −0.413899641 |
| DO 897/99 | −0.413899641 |
| Hydrastine hydrochloride | −0.413899641 |
| Vitamin K2 | −0.414119978 |
| Verapamyl hydrochloride | −0.416144086 |
| Piroxicam | −0.416144086 |
| Reserpinic acid hydrochloride | −0.427769291 |
| Sulfanilamide | −0.432451029 |
| Zaprinast | −0.434141605 |
| Todralazine hydrochloride | −0.444428665 |
| Tetrandrine | −0.453756608 |
| Fluvastatin sodium salt | −0.453756608 |
| Amikacin hydrate | −0.454275864 |
| Syrosingopine | −0.454275864 |
| Ethamsylate | −0.454921686 |
| Etilefrine hydrochloride | −0.454921686 |
| Methacycline hydrochloride | −0.468422606 |
| Pronethalol hydrochloride | −0.468422606 |
| Aminocaproic acid | −0.474937842 |
| Adamantamine fumarate | −0.494207481 |
| Cefotetan | −0.494207481 |
| Letrozole | −0.495268824 |
| Meprylcaine hydrochloride | −0.495268824 |
| Gabapentin | −0.495990838 |
| Prazosin hydrochloride | −0.495990838 |
| Minaprine dihydrochloride | −0.50034513 |
| Fexofenadine HCl | −0.508612222 |
| Butirosin disulfate salt | −0.516911967 |
| Citalopram Hydrobromide | −0.516911967 |
| Chlorotrianisene | −0.516911967 |
| Chloropyramine hydrochloride | −0.516911967 |
| S(−)Eticlopride hydrochloride | −0.516911967 |
| Cyproterone acetate | −0.521901282 |
| Tolfenamic acid | −0.52795945 |
| Hyoscyamine (L) | −0.52795945 |
| Betaxolol hydrochloride | −0.533669016 |
| Azathymine, 6 | −0.533669016 |
| Pyridoxine hydrochloride | −0.533669016 |
| Levopropoxyphene napsylate | −0.535135166 |
| Ramipril | −0.535135166 |
| Dihydroergotamine tartrate | −0.541677183 |
| Oleandomycin phosphate | −0.541677183 |
| Chlormezanone | −0.553216451 |
| Ceforanide | −0.553216451 |
| Praziquantel | −0.555334115 |
| Beta-Escin | −0.558345396 |
| Pempidine tartrate | −0.558345396 |
| Asiaticoside | −0.561434237 |
| Cyclopentolate hydrochloride | −0.561434237 |
| Diphenhydramine hydrochloride | −0.568929421 |
| Bepridil hydrochloride | −0.572638313 |
| Lymecycline | −0.574497949 |
| Mesalamine | −0.574497949 |
| Ioversol | −0.578851723 |
| Ronidazole | −0.587503531 |
| Bambuterol hydrochloride | −0.591932338 |
| Artemisinin | −0.591932338 |
| Nisoldipine | −0.599214226 |
| Ganciclovir | −0.599214226 |
| Iopromide | −0.599214226 |
| Prednisone | −0.609338028 |
| Quinidine hydrochloride monohydrate | −0.609338028 |
| Ajmalicine hydrochloride | −0.611096459 |
| Tropine | −0.613337792 |
| Cefazolin sodium salt | −0.619430539 |
| Hexylcaine hydrochloride | −0.619430539 |
| Clofilium tosylate | −0.622679511 |
| Betahistine mesylate | −0.6301288 |
| Dacarbazine | −0.6301288 |
| Griseofulvin | −0.635955938 |
| Ticarcillin sodium | −0.638932403 |
| Timolol maleate salt | −0.639498529 |
| Suloctidil | −0.649027668 |
| Ipratropium bromide | −0.649027668 |
| Neomycin sulfate | −0.649166759 |
| Dyclonine hydrochloride | −0.649166759 |
| Levocabastine hydrochloride | −0.651638562 |
| Oxybenzone | −0.659416296 |
| Bufexamac | −0.662311465 |
| Chlorprothixene hydrochloride | −0.667791554 |
| Succinylsulfathiazole | −0.67918216 |
| Pregnenolone | −0.686419124 |
| Pseudopelletierine hydrochloride | −0.686419124 |
| Ethotoin | −0.698794657 |
| Urosiol | −0.698794657 |
| Imipramine hydrochloride | −0.701344446 |
| Oxybutynin chloride | −0.701344446 |
| Nialamide | −0.701845586 |
| Tacrine hydrochloride hydrate | −0.704909209 |
| Arecoline hydrobromide | −0.704909209 |
| Cephalosporanic acid, 7-amino | −0.704909209 |
| Prenylamine lactate | −0.704909209 |
| Amitryptiline hydrochloride | −0.714220441 |
| 6-Furfurylaminopurine | −0.718252533 |
| Pridinol methanesulfonate salt | −0.718252533 |
| Ascorbic acid | −0.723260807 |
| Cefoperazone dihydrate | −0.723260807 |
| Tocopherol (R,S) | −0.723260807 |
| Cefaclor | −0.723260807 |
| Harmol hydrochloride monohydrate | −0.723260807 |
| Prednicarbate | −0.726573636 |
| Glafenine hydrochloride | −0.727028393 |
| Skimmianine | −0.737554726 |
| Diphenylpyraline hydrochloride | −0.737554726 |
| Atractyloside potassium salt | −0.737554726 |
| Cefamandole sodium salt | −0.737554726 |
| Esculetin | −0.737554726 |
| (Ã±)-Nipecotic acid | −0.737554726 |
| Clocortolone pivalate | −0.738842804 |
| Ketotifen fumarate | −0.74147307 |
| Roxarsone | −0.751048441 |
| Homosalate | −0.751048441 |
| Oxymetazoline hydrochloride | −0.75243911 |
| Merbromin | −0.756700371 |
| Propidium iodide | −0.756700371 |
| Arcaine sulfate | −0.756700371 |
| Altretamine | −0.756700371 |
| Tropisetron HCl | −0.759545301 |
| Adiphenine hydrochloride | −0.765041438 |
| Ursolic acid | −0.765041438 |
| Ribostamycin sulfate salt | −0.775688779 |
| 3-Acetylcoumarin | −0.775688779 |
| Prochlorperazine dimaleate | −0.777476946 |
| Mycophenolic acid | −0.777476946 |
| Diperodon hydrochloride | −0.777574846 |
| Glibenclamide | −0.777574846 |
| Trifluridine | −0.787282485 |
| Dorzolamide hydrochloride | −0.787282485 |
| Lithocholic acid | −0.794519436 |
| Luteolin | −0.794519436 |
| Estradiol-17 beta | −0.795267589 |
| Harmalol hydrochloride dihydrate | −0.795267589 |
| Talampicillin hydrochloride | −0.811118266 |
| Oxyphenbutazone | −0.811118266 |
| Pentamidine isethionate | −0.812916944 |
| Cefuroxime sodium salt | −0.813191993 |
| Propoxycaine hydrochloride | −0.822939863 |
| Josamycin | −0.827017008 |
| Xylometazoline hydrochloride | −0.827017008 |
| Phenylpropanolamine hydrochloride | −0.830424846 |
| Vigabatrin | −0.830424846 |
| Stachydrine hydrochloride | −0.830424846 |
| Benzocaine | −0.831706256 |
| Clofibric acid | −0.831706256 |
| Dichlorphenamide | −0.831706256 |
| Etidronic acid, disodium salt | −0.831706256 |
| Harpagoside | −0.834697224 |
| Dipyridamole | −0.83920438 |
| Mifepristone | −0.83920438 |
| Methyldopate hydrochloride | −0.846390352 |
| Zidovudine, AZT | −0.847791252 |
| Thyroxine (L) | −0.847791252 |
| Brompheniramine maleate | −0.847791252 |
| Dosulepin hydrochloride | −0.847791252 |
| Glycocholic acid | −0.847791252 |
| Terfenadine | −0.851322466 |
| Cinnarizine | −0.851322466 |
| Maprotiline hydrochloride | −0.865016226 |
| Fluoxetine hydrochloride | −0.865016226 |
| Lovastatin | −0.865016226 |
| Thiamine hydrochloride | −0.865016226 |
| Doxazosin mesylate | −0.868259845 |
| Gentamicine sulfate | −0.875350938 |
| Methiothepin maleate | −0.88209994 |
| Alcuronium chloride | −0.892521299 |
| Proglumide | −0.899103001 |
| Enalapril maleate | −0.899103001 |
| Azaperone | −0.903894021 |
| Sulfabenzamide | −0.904181431 |
| Zomepirac sodium salt | −0.904181431 |
| Securinine | −0.904181431 |
| Lomefloxacin hydrochloride | −0.910875659 |
| Naftopidil dihydrochloride | −0.926448239 |
| Quinethazone | −0.926448239 |
| Penbutolol sulfate | −0.937630034 |
| Piretanide | −0.937630034 |
| Methylhydantoin-5-(D) | −0.9394742 |
| Simvastatin | −0.9394742 |
| (R)-(+)-Atenolol | −0.9394742 |
| Tracazolate hydrochloride | −0.948748512 |
| Zalcitabine | −0.948748512 |
| Isocarboxazid | −0.956886246 |
| Memantine Hydrochloride | −0.959803859 |
| Nadifloxacin | −0.959803859 |
| Metixene hydrochloride | −0.965412134 |
| Atropine sulfate monohydrate | −0.965412134 |
| Delcorine | −0.974143041 |
| Finasteride | −0.974143041 |
| Fluorometholone | −0.974143041 |
| Meglumine | −0.974143041 |
| Cyclosporin A | −0.981656974 |
| Pantothenic acid calcium salt monohydrate | −0.981656974 |
| Mephenytoin | −0.981725996 |
| Cefepime hydrochloride | −0.992593229 |
| Selegiline hydrochloride | −0.997764184 |
| Flufenamic acid | −1.002599411 |
| Orphenadrine hydrochloride | −1.002599411 |
| Dipyrone | −1.00819432 |
| Methotrimeprazine maleat salt | −1.00819432 |
| Tyloxapol | −1.00819432 |
| Desipramine hydrochloride | −1.013734565 |
| Testosterone propionate | −1.013734565 |
| Coralyne chloride hydrate | −1.013734565 |
| Ketoprofen | −1.013761143 |
| Kaempferol | −1.014141243 |
| Betulin | −1.024822541 |
| Piperidolate hydrochloride | −1.024822541 |
| Alverine citrate salt | −1.024856283 |
| Amrinone | −1.024990712 |
| Iopamidol | −1.024990712 |
| Lidoflazine | −1.029568975 |
| Reserpine | −1.041635659 |
| Oxprenolol hydrochloride | −1.046001098 |
| Sotalol hydrochloride | −1.056498939 |
| Flucloxacillin sodium | −1.056498939 |
| Pramoxine hydrochloride | −1.058130276 |
| Alfuzosin hydrochloride | −1.060833561 |
| Chlorthalidone | −1.060833561 |
| Metampicillin sodium salt | −1.068577294 |
| Paromomycin sulfate | −1.07447574 |
| Albendazole | −1.07934434 |
| Benzethonium chloride | −1.090673269 |
| GBR 12909 dihydrochloride | −1.091565752 |
| Trihexyphenidyl-D, L Hydrochloride | −1.106724131 |
| Bendroflumethiazide | −1.106724131 |
| alpha-Santonin | −1.10808616 |
| Haloperidol | −1.1112595 |
| Cefotiam hydrochloride | −1.121773981 |
| Harmine hydrochloride | −1.121773981 |
| Dienestrol | −1.122629632 |
| Theophylline monohydrate | −1.122629632 |
| Zardaverine | −1.128306257 |
| Ifenprodil tartrate | −1.132218404 |
| Ketanserin tartrate hydrate | −1.13668439 |
| Proadifen hydrochloride | −1.138391116 |
| Paroxetine Hydrochloride | −1.138391116 |
| Ozagrel hydrochloride | −1.14829346 |
| Piperacetazine | −1.14829346 |
| Butoconazole nitrate | −1.151467197 |
| Camptothecine (S, +) | −1.16318781 |
| Tubocurarine chloride pentahydrate (+) | −1.166123587 |
| Naftifine hydrochloride | −1.168050643 |
| Methyl benzethonium chloride | −1.169487558 |
| Theobromine | −1.169487558 |
| Pimozide | −1.173387513 |
| Cloperastine hydrochloride | −1.184825345 |
| Serotonin hydrochloride | −1.195061932 |
| Doxorubicin hydrochloride | −1.20934634 |
| Scoulerine | −1.20934634 |
| Dibucaine | −1.21358027 |
| Repaglinide | −1.21645666 |
| Thioproperazine dimesylate | −1.223479067 |
| Ethoxyquin | −1.23001438 |
| Pimethixene maleate | −1.233318965 |
| Terbutaline hemisulfate | −1.237551851 |
| Ofloxacin | −1.243100397 |
| Nimodipine | −1.244807546 |
| Oxethazaine | −1.252823798 |
| Chlorambucil | −1.263490751 |
| Methiazole | −1.263490751 |
| Androsterone | −1.26528137 |
| Bromocryptine mesylate | −1.272098252 |
| Benzydamine hydrochloride | −1.272098252 |
| Famprofazone | −1.288398494 |
| Piribedil hydrochloride | −1.291072796 |
| Disulfiram | −1.291145823 |
| Benzylpenicillin sodium | −1.300162753 |
| Deoxycorticosterone | −1.302670998 |
| Furosemide | −1.306005445 |
| Phthalylsulfathiazole | −1.31681712 |
| Pyrvinium pamoate | −1.318188927 |
| Minocycline hydrochloride | −1.319299449 |
| Pepstatin A | −1.319353197 |
| Dipivefrin hydrochloride | −1.319353197 |
| Nocodazole | −1.328574348 |
| Loperamide hydrochloride | −1.328574348 |
| Salmeterol | −1.330838373 |
| (−)-Eseroline fumarate salt | −1.336012721 |
| Zuclopenthixol hydrochloride | −1.344849701 |
| Carbarsone | −1.345715958 |
| Rimexolone | −1.353637256 |
| Cephalothin sodium salt | −1.358512311 |
| Streptomycin sulfate | −1.358733516 |
| Azaguanine-8 | −1.365137394 |
| Levodopa | −1.365137394 |
| Hexetidine | −1.37164355 |
| Sertraline | −1.388300962 |
| Methocarbamol | −1.392008457 |
| Fluspirilen | −1.3991243 |
| Propranolol hydrochloride | −1.412427866 |
| Paclitaxel | −1.418419954 |
| Halofantrine hydrochloride | −1.422204673 |
| Ciclopirox ethanolamine | −1.422233577 |
| Crotamiton | −1.425617001 |
| Meclofenamic acid sodium salt monohydrate | −1.427123646 |
| Isosorbide dinitrate | −1.438693161 |
| Dilazep dihydrochloride | −1.444383344 |
| Benzdromarone | −1.459108182 |
| Flumequine | −1.461448902 |
| Thiethylperazine malate | −1.463552446 |
| Megestrol acetate | −1.464512313 |
| Denatonium benzoate | −1.471688135 |
| Fendiline hydrochloride | −1.478323615 |
| Triflupromazine hydrochloride | −1.51151347 |
| Benzamil hydrochloride | −1.514857524 |
| Enilconazole | −1.535228015 |
| Liothyronine | −1.539405689 |
| Proscillaridin A | −1.542982587 |
| Parbendazole | −1.542982587 |
| 3-Acetamidocoumarin | −1.550696553 |
| Verteporfin | −1.550696553 |
| Scopoletin | −1.558370253 |
| Decamethonium bromide | −1.558370253 |
| Sanguinarine | −1.558370253 |
| Tribenoside | −1.558370253 |
| Tridihexethyl chloride | −1.558370253 |
| Sertaconazole nitrate | −1.558370253 |
| Etoposide | −1.575852679 |
| Mebendazole | −1.583577746 |
| Fenbendazole | −1.60683165 |
| Amiodarone hydrochloride | −1.609143015 |
| Amprolium hydrochloride | −1.614500847 |
| Atovaquone | −1.62007216 |
| Chlorpromazine hydrochloride | −1.622129585 |
| Mefloquine hydrochloride | −1.637267081 |
| Clomipramine hydrochloride | −1.637267081 |
| Homochlorcyclizine dihydrochloride | −1.659678542 |
| Perhexiline maleate | −1.681744353 |
| Alexidine dihydrochloride | −1.73284611 |
| Perphenazine | −1.745955303 |
| Mephenesin | −1.752912664 |
| Nortriptyline hydrochloride | −1.752912664 |
| Chlorcyclizine hydrochloride | −1.753970394 |
| Clomiphene citrate (Z,E) | −1.755036611 |
| Zoxazolamine | −1.764903975 |
| Clofazimine | −1.764903975 |
| Amodiaquin dihydrochloride dihydrate | −1.766724674 |
| Primaquine diphosphate | −1.784432293 |
| Antimycin A | −1.787190234 |
| Niclosamide | −1.7939459 |
| Colchicine | −1.803690901 |
| Thalidomide | −1.820645004 |
| Meclozine dihydrochloride | −1.822686234 |
| Chrysene-1,4-quinone | −1.822686234 |
| Raloxifene hydrochloride | −1.825488704 |
| Tamoxifen citrate | −1.827240241 |
| Betulinic acid | −1.832087132 |
| Econazole nitrate | −1.833804191 |
| Promazine hydrochloride | −1.845263647 |
| Lynestrenol | −1.846839275 |
| Fluphenazine dihydrochloride | −1.846839275 |
| Astemizole | −1.866163686 |
| Ivermectin | −1.866163686 |
| Trifluoperazine dihydrochloride | −1.866163686 |
| Miconazole | −1.8725453 |
| Tomatine | −1.8725453 |
| Acemetacin | −1.8725453 |
| Thonzonium bromide | −1.883982049 |
| Clemastine fumarate | −1.891514003 |
| Thioridazine hydrochloride | −1.897778989 |
| Flunarizine dihydrochloride | −1.897778989 |
| Quinacrine dihydrochloride dihydrate | −1.897778989 |
| R(−) Apomorphine hydrochloride hemihydrate | −1.904015411 |
| Isoconazole | −1.910223501 |
| Clotrimazole | −1.910223501 |
| Menadione | −1.948784318 |
| Ellipticine | −1.948784318 |
| Emetine dihydrochloride | −1.991134395 |
| Daunorubicin hydrochloride | −1.999447482 |
| Cycloheximide | −2.002731668 |
| Digitoxigenin | −2.007709622 |
| Piperlongumine | −2.024083319 |
| Digoxin | −2.04025992 |
| Strophantine octahydrate | −2.04025992 |
| Anisomycin | −2.056243732 |
| Puromycin dihydrochloride | −2.056243732 |
| Azacytidine-5 | −2.143722458 |
| Podophyllotoxin | −2.151569024 |
| Cantharidin | −2.174833466 |
| Lycorine hydrochloride | −2.212719429 |
| Meclofenoxate hydrochloride | −2.263991749 |
| Monensin sodium salt | −2.299424917 |
| Lanatoside C | −2.333922611 |
| Gossypol | −2.367530607 |
| Lasalocid sodium salt | −2.367530607 |
| Digoxigenin | −2.387286222 |
| Strophanthidin | −2.400291577 |

We first predicted drug protein interactions for the entire Prestwick library using the similarity ensemble approach (SEA)(Keiser et al., 2007). We then added normalized z-score across all compounds that target each protein to calculate weighted combined z-score for each protein and used it for iPAGE GO analysis (Goodarzi et al., 2009). Among the GO terms associated with the SC protecting drug candidates we identified oxidative phosphorylation (OXPHOS), nitrogen metabolism and metallopeptidases (FIG. 3F). We validated the expression of these candidate pathways in our HP cultures using module scoring analysis (FIG. S8B). To determine the degree to which positive z-scores were enriched among the drugs targeting each protein, we performed a Fisher's exact test. Through this analysis, we identified 33 proteins as significant drug targets filtered based on average combined z-score>0, false discovery rate (FDR)<0.25 and Fisher's p<0.1 (FIG. 3 G-I).

We performed a protein-protein interaction network analysis to identify interactions between our 33 significant drug targets using the STRING database (Szklarczyk et al., 2019). In the resulting network, IL6, NR3C1, PGR and PTGS2 had the highest degree centrality (FIG. 3J). In addition, for a more comprehensive target prediction, we generated a list of potential targets for the top hits derived from the HTS dataset (FIG. S8C) or computational predictions that use network-based and similarity-based algorithms (FIG. 3K, Table S5). Intriguingly, many of the potential target proteins were shared between multiple hits; including potassium channels (KCNs), estrogen and progesterone receptors (ESRs and PGRs), prostaglandin synthases (PTGSs) and prostaglandin receptors (PTGERs). Notably, the predicted target pattern of tolbutamide resembled that of our top hit, bupropion (BP) (FIG. 3K).

TABLE S5

| | | | | | Furaltadone | | |
|---|---|---|---|---|---|---|---|
| Bupropion | Cyclopenthiazide | Niradizole | Gliquidone | Condelphine | HCl | Captopril | Nimesulide |
| Abat | ca1 | CYP1A2 | ABCB11 | CHRNA7 | CISD1 | ABCB1 | ABCB1 |
| ABCB1 | Ca13 | CYP3A4 | ABCC2 | | MAPK8 | ABCB11 | ABCC8 |
| ABCB11 | CA5A | DRD2 | ABCC4 | | MAPK9 | ABCC1 | ABCC9 |
| ADRA1A | CA5B | HCRTR1 | ABCC8 | | MITF | ABCC2 | APEX1 |
| ADRA2A | CA6 | HSD17B10 | APEX1 | | | ABCC3 | BRD1 |
| ADRB1 | Gria2 | HTT | AR | | | ABCC4 | BRD4 |
| AKR1C2 | KPNB1 | SMN1 | Chain A, Crystal Structure Of The Human | | | ACE | BRPF1 |
| | | | 2-oxoglutarate Oxygenase Loc390245 | | | | |
| | | | (human) | | | | |
| AKR1C3 | SNUPN | SMN2 | CYP2C9 | | | Ace2 | CFTR |
| ALDH1A1 | | TNNC1 | CYP3A4 | | | ALB | CTSV |
| CHRM1 | | TNNI3 | ESR2 | | | ANPEP | CYP19A1 |
| CHRM2 | | TNNT2 | ESRRA | | | CYP2D6 | CYP1A2 |
| CHRM3 | | TPM1 | HIF1A | | | DAPE | CYP2C18 |
| CHRM4 | | TSHR | HPGD | | | DPP4 | CYP2C19 |
| CHRM5 | | | HSD17B10 | | | DRD2 | CYP2C8 |
| CHRNA1 | | | KCNJ11 | | | ECE1 | CYP2C9 |
| CHRNA3 | | | KCNJ8 | | | ECE2 | CYP2D6 |
| CHRNA4 | | | KDM4E | | | EHMT2 | CYP3A4 |
| CHRNA7 | | | LMNA | | | F10 | EHMT2 |
| CHRNB1 | | | NFE2L2 | | | F2 | ESRRA |
| CHRNB2 | | | NR1H4 | | | IMP1 | FBP1 |
| CHRNB4 | | | NR1I2 | | | KDM4E | HIF1A |
| CHRND | | | PGR | | | LACTB | KCNJ1 |
| CHRNG | | | PPARG | | | LACTB2 | KCNMA1 |
| CYP1A2 | | | | | | LMNA | KMT2A |
| CYP2A6 | | | | | | LTA4H | LMNA |
| CYP2B6 | | | | | | MAPT | LTA4H |
| CYP2C19 | | | | | | MME | LTF |
| CYP2C8 | | | | | | MMP12 | MAPT |
| CYP2C9 | | | | | | MMP2 | MBNL1 |
| CYP2D6 | | | | | | MMP9 | MEN1 |
| CYP2E1 | | | | | | PREP | MMP1 |
| CYP3A4 | | | | | | PRSS1 | MPO |
| DHX8 | | | | | | PSIP1 | PGR |
| DRD2 | | | | | | PTGS1 | PLA2G2E |
| EIF4G2 | | | | | | PTGS2 | PPARG |
| ELK3 | | | | | | REN | PTGS1 |
| GABRA1 | | | | | | SLC15A1 | PTGS2 |
| GPR139 | | | | | | SLC22A6 | Rorc |
| Grm5 | | | | | | Tpp2 | SLC15A1 |
| HRH1 | | | | | | VIM2 | SLC15A2 |
| HTR2A | | | | | | XIAP | SLC22A6 |
| HTR2C | | | | | | XPNPEP2 | SLC6A3 |
| HTT | | | | | | | SLCO1A2 |
| ITGA5 | | | | | | | SLCO2B1 |
| JUN | | | | | | | SMN1 |
| KCNK9 | | | | | | | SMN2 |
| Kmo | | | | | | | TBXA2R |
| LMO3 | | | | | | | TDP1 |
| NAT1 | | | | | | | TRIM24 |
| NET1 | | | | | | | TSHR |
| ORM1 | | | | | | | |
| PRPF38B | | | | | | | |
| PTGER4 | | | | | | | |
| SLC12A5 | | | | | | | |
| SLC6A2 | | | | | | | |
| SLC6A3 | | | | | | | |
| SLC6A4 | | | | | | | |
| SLC6A5 | | | | | | | |
| TDP1 | | | | | | | |
| UGT1A1 | | | | | | | |
| UGT1A4 | | | | | | | |
| UGT1A6 | | | | | | | |
| UGT2B10 | | | | | | | |
| UGT2B7 | | | | | | | |

| Nafronyl | | | | Spaglumic | (d,l)- | | |
|---|---|---|---|---|---|---|---|
| oxalate | Tolbutamide | Perindopril | Trolox | acid | Tetrahydroberberine | Ribivarin | Triamcinolone |
| ABCB1 | ACAT | ABCB11 | ACHE | Ace | ache | ABCB11 | ABCB11 |
| ABCB11 | ADRB3 | ACE | BCHE | ANPEP | ADRA1A | ABCC2 | ABCC1 |
| Amy2 | ALB | MME | DHRS9 | CAPN1 | ADRA1D | ABCC3 | ACHE |
| BAZ2B | ALOX15 | SLC15A1 | EHMT2 | CASP1 | ADRA2A | ABCC4 | AR |
| Chain A, | BSEP | SLC15A2 | Ptger2 | CASP3 | ADRA2B | ADA | BLM |
| Crystal | | | | | | | |
| Structure Of | | | | | | | |
| The Human | | | | | | | |
| 2- | | | | | | | |
| oxoglutarate | | | | | | | |
| Oxygenase | | | | | | | |
| Loc390245 | | | | | | | |
| (human) | | | | | | | |
| CHRM1 | CA1 | | Ptger4 | CASP7 | ADRA2C | ADK | CFTR |
| CHRM2 | CA12 | | PTGS1 | CASP8 | AHR | ADORA1 | CHRM1 |
| CHRM4 | CA14 | | PTGS2 | CFB | AR | ADORA2A | CHRM5 |
| CYP3A4 | CA2 | | TTPA | cpg2 | BCHE | ADORA3 | CTSL |
| Ghsr | CA4 | | TYR | DLG4 | CDK2 | AHCY | CYP1A1 |
| HTR2A | CASA | | XDH | EDNRA | CHRM4 | AK | CYP1A2 |
| JUN | CA7 | | | Ednrb | Chrnb2 | ALDH1A1 | CYP219 |
| KCNH2 | CA9 | | | ENPEP | Chrnb4 | AR | CYP2C19 |
| KDM4A | CH25H | | | FOLH1 | CYP1A2 | ATIC | CYP2C9 |
| KDM4E | COQ6 | | | FOLH2 | CYP2C19 | ca1 | CYP2D6 |
| KMT2A | CYP11A1 | | | GGH | CYP2D6 | CA12 | CYP2J2 |
| MEN1 | CYP11B1 | | | GNPAT | CYP3A4 | CA14 | CYP3A4 |
| PANC | CYP11B2 | | | GRIA3 | DPP4 | CA2 | ESR1 |
| | CYP17A1 | | | HPN | DRD1 | CA9 | FABP1 |
| | CYP19A1 | | | ITGA2B | DRD2 | CDA | GLUL |
| | CYP1A1 | | | ITGB3 | DRD3 | CXCL8 | HIF1A |
| | CYP1A2 | | | KEAP1 | DRD4 | CYP3A4 | HSD17B10 |
| | CYP1B1 | | | MME | DRD5 | ENPP1 | IL6 |
| | CYP20A1 | | | MMP10 | ESRRA | ESR1 | LMNA |
| | CYP21A2 | | | MMP12 | F3 | GAPDH | MMP1 |
| | CYP24A1 | | | MMP13 | F7 | GRK1 | NEF2L2 |
| | CYP26A1 | | | MMP2 | GLI3 | GSK3B | NFKB1 |
| | CYP26B1 | | | MMP3 | GPR32 | H2AX | NR3C1 |
| | CYP26C1 | | | MMP8 | HTR1A | HSPA5 | PGR |
| | CYP27A1 | | | NAALAD2 | HTR2A | HSPA8 | PTGS2 |
| | CYP27B1 | | | PTPRC | HTR2B | IMPDH1 | SERPINA6 |
| | CYP2A13 | | | RIR1 | HTR7 | IMPDH2 | |
| | CYP2A6 | | | Rrm1 | NR1I3 | LMNA | |
| | CYP2A7 | | | SIRT5 | PGR | NR1I2 | |
| | CYP2B6 | | | SLC15A2 | RAD52 | NR1I3 | |
| | CYP2C18 | | | SLC36A1 | SIGMAR1 | NT5C2 | |
| | CYP2C19 | | | SPSB2 | SLC22A2 | PAX8 | |
| | CYP2C8 | | | SRC | TDP1 | PDCD4 | |
| | CYP2C9 | | | WDR5 | | PYGM | |
| | CYP2D6 | | | | | RORC | |
| | CYP2D7 | | | | | SLC28A1 | |
| | CYP2E1 | | | | | SLC28A2 | |
| | CYP2F1 | | | | | SLC28a3 | |
| | CYP2J2 | | | | | SLC29A1 | |
| | CYP2R1 | | | | | SLC29A2 | |
| | CYP2S1 | | | | | SMN1 | |
| | CYP2U1 | | | | | SMN2 | |
| | CYP2W1 | | | | | TDP1 | |
| | CYP39A1 | | | | | THPO | |
| | CYP3A4 | | | | | THRB | |
| | CYP3A43 | | | | | TP53 | |
| | CYP3A5 | | | | | TSHR | |
| | CYP3A7 | | | | | | |
| | CYP3A7-CYP3A51P | | | | | | |
| | CYP46A1 | | | | | | |
| | CYP4A11 | | | | | | |
| | CYP4A22 | | | | | | |
| | CYP4B1 | | | | | | |
| | CYP4F11 | | | | | | |
| | CYP4F12 | | | | | | |
| | CYP4F2 | | | | | | |
| | CYP4F22 | | | | | | |
| | CYP4F3 | | | | | | |
| | CYP4F8 | | | | | | |
| | CYP4V2 | | | | | | |
| | CYP4X1 | | | | | | |
| | CYP4Z1 | | | | | | |
| | CYP51 | | | | | | |
| | CYP51A1 | | | | | | |
| | CYP7A1 | | | | | | |
| | CYP7B1 | | | | | | |
| | CYP8B1 | | | | | | |
| | DRD2 | | | | | | |
| | EPHX1 | | | | | | |
| | EPHX2 | | | | | | |
| | FBP1 | | | | | | |
| | GHSR | | | | | | |
| | Kcnj11 | | | | | | |
| | KCNJ8 | | | | | | |
| | LMNA | | | | | | |
| | MMP12 | | | | | | |
| | MMP14 | | | | | | |
| | MMP2 | | | | | | |
| | MMP8 | | | | | | |
| | MMP9 | | | | | | |
| | NS5B | | | | | | |
| | PTGER1 | | | | | | |
| | PTGER4 | | | | | | |
| | PTGIS | | | | | | |
| | PTGS2 | | | | | | |
| | SLC22A6 | | | | | | |
| | SLCO1A2 | | | | | | |
| | SLCO2B1 | | | | | | |
| | TASP1 | | | | | | |
| | TBXA2R | | | | | | |
| | TDP1 | | | | | | |
| | TREK1 | | | | | | |
| | UGT1A1 | | | | | | |
| | UGT1A4 | | | | | | |
| | UGT1A6 | | | | | | |
| | UGT2B10 | | | | | | |
| | UGT2B7 | | | | | | |

Our top hit compound, BP, is a widely used antidepressant marketed as Wellbutrin®. BP showed a dose-dependent effect on rescuing the viability of high glucose treated SC cultures (FIG. S8D). In line with previous reports on glucose-mediated activation of oxidative stress response (Correa-Silva et al., 2018; Kowluru et al., 2003), we observed the activation of cellular inflammatory response via NF-kB p65 localization to the nucleus in SCs exposed to high glucose, a phenotype that was counteracted by BP treatment (FIG. S8E).

To understand the mechanism of glucotoxicity in SC cultures and its rescue with BP, we performed unbiased transcriptional and metabolite profiling (FIG. 3L). We compared gene expression profiles of SCs treated with low glucose and high glucose with or without BP treatment followed by pathway enrichment analysis of differentially expressed genes. Of the 1,559 SC transcripts that were enriched or depleted in response to high glucose, 66 showed reversed expression pattern in cells treated with BP (FIG. S9A). Of these, PTGER4 was the only gene in common with the list of predicted targets of BP (FIG. 3 I and K, Table S5). The affected processes in cells exposed to high glucose included several metabolic and cell cycle signaling pathways and BP treatment modulated pathways related to DNA replication and transcription as well as cell cycle and stress response.

To better understand the metabolic consequences of high glucose and BP treatments, we performed metabolomics and integrated the data with our bulk RNA sequencing results. The detected primary metabolites fell into seven categories based on their pattern of abundance in response to glucose and BP (FIG. S10 A and B). Many of the metabolites were either accumulated or depleted in SCs exposed to high glucose. BP treatment led to a reversed response in a subset of these metabolites in particular groups 4 and 7. Metabolic pathway enrichment analysis suggested modulations in citric acid cycle, urea cycle, amino acid metabolism, glycolysis and gluconeogenesis (FIG. S10 A and B). In parallel, BP treatment led to an increase in the detected levels of TCA cycle metabolites succinate, fumarate, citrate, α-ketoglutarate and malate (FIG. S10, FIG. S11A). The concentration of citrate was reduced in response to high glucose and was reversed by BP treatment. Citrate transporter SLC13A2 expression followed the same trend (FIG. S10, FIG. S11A). Exposure to high glucose resulted in elevation of cellular urea that was accompanied by increased transcript levels of the plasma membrane urea transporter SLC14A2 (FIG. S10, FIG. S11A). Both urea concentration and its transporter mRNA level were decreased in the presence of BP (FIG. S10, FIG. S11A). We observed an increase in cellular pyruvate in response to high glucose that was lowered by BP treatment (FIG. S10, FIG. S11A). Cellular lactate level was reduced after BP treatment which was accompanied by downregulation of membrane monocarboxylate transporters SLC16A3, SLC16A14, SLC5A2 RNA levels (FIG. S10, FIG. S11A). It has been previously reported that in some cell types elevated glucose levels can activate the polyol pathway (Oates, 2002). The polyol pathway metabolizes excess intracellular glucose into sorbitol and subsequently into fructose via two enzymatic steps catalyzed by aldose reductase (AR) and sorbitol dehydrogenase (SDH), respectively. Osmotic and oxidative stress triggered by the polyol pathway have been proposed as mediators of tissue damage in response to high glucose based on studies in the lens more than 50 years ago (Van Heyningen, 1959). Sorbitol accumulation has been implicated in peripheral nerve damage in multiple animal models of diabetes (Mizisin, 2014; Oates, 2002). We asked whether hPSC-SCs show increased sorbitol levels in response to high glucose as a potential mechanism of their selective vulnerability. In agreement with studies in the mouse (Maekawa et al., 2001; Mizisin and Powell, 1993), we observed a higher AR to SDH ratio in hPSC-derived SCs compared to sensory neurons (FIG. S11B). Furthermore, SCs but not sensory neurons showed increased levels of sorbitol when exposed to high glucose (FIG. S11C). BP treatment reduced sorbitol accumulation in SCs in a dose-dependent manner (FIG. S11D). Similarly, we observed an increase in the cellular level of fructose in SCs treated with high glucose and BP treatment countered this effect (FIG. SHE). This is in agreement with the protective effect of aldose reductase inhibitors in diabetic SCs (Hao et al., 2015). Collectively, these results point to a global metabolic shift in high glucose treated SCs that is reversed by BP treatment.

Pathway enrichment analysis of transcripts that were upregulated in response to high glucose and reversed in response to BP revealed glycerolipid metabolism and ErbB signaling pathways (FIG. 3M). Transcriptional changes in the glycerolipid metabolism pathway (FIG. 3N) were accompanied by corresponding changes in pathway metabolites measured in the metabolomics dataset (FIG. 3O). Increased tri- and diacylglycerol degradation into free fatty acid and glycerol was suggested by upregulation of LIPG and PNLIPRP3 transcripts in response to high glucose treatment (FIG. 3N). This is interesting given the crucial role of lipid metabolism in myelin production and SC physiology (FIG. S3H). Further, through the action of prostaglandin synthases, the glycerolipid metabolic pathway is directly linked to the prostaglandin metabolism. This piqued our interest for multiple additional reasons. For example, prostaglandin E2 receptor (PTGER4) and prostaglandin-endoperoxide synthase 2 (PTGS2) were among the top significant protein targets identified by our high-throughput drug screening (FIG. 3I). Remarkably, they were both part of the protein-protein interaction network with PTGS2 showing a high degree network centrality (FIG. 3J) and PTGSs and PTGERs were part of the protein families shared between our top drug hits (FIG. 3K). Moreover, PTGER4 transcript had a reversed pattern of expression in response to high glucose in presence and absence of BP (FIG. S9A). We aimed to investigate the effect of PTGER4 in SC glucotoxicity using a genetic approach. We knocked out PTGER4 in the Schwann cells using CRISPR-Cas9 ribonucleoproteins (RNP) and observed a significant reduction in cleaved caspase-3 in response to high glucose treated cells indicating a lower sensitivity to glucotoxicity in the absence of PTGER4 (FIG. 3P).

### Buproprion Rescues the Disease Phenotypes in a Mouse Model of DPN

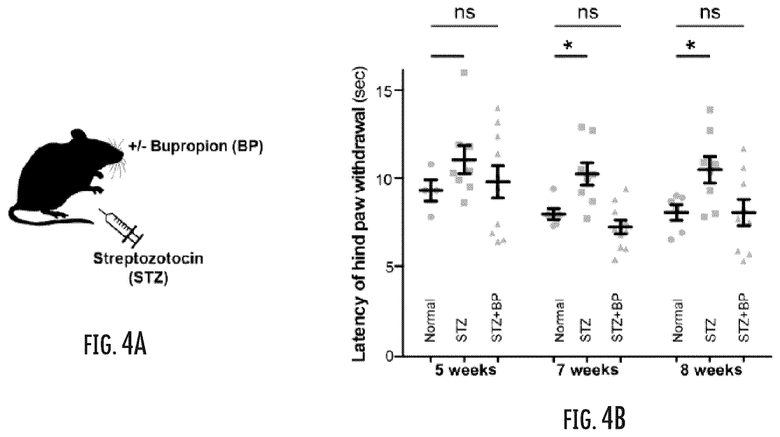

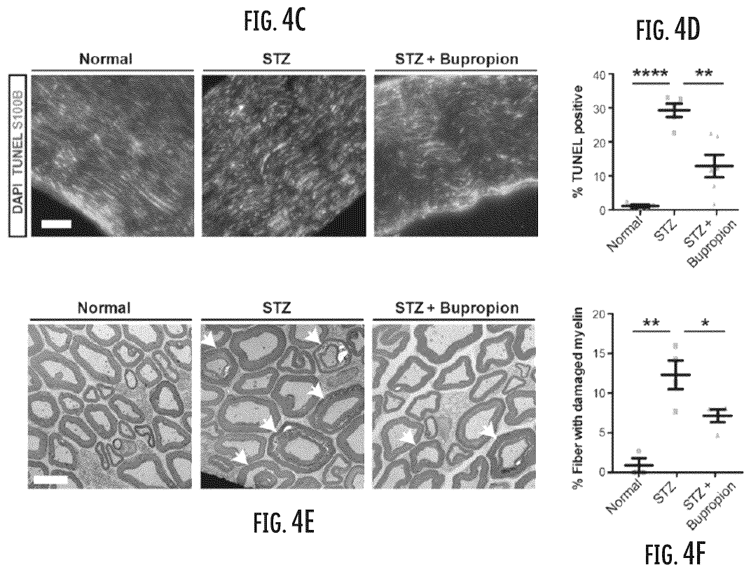

Given the remarkable ability of BP in rescuing the viability of hPSC-SCs in vitro, we next assessed the therapeutic potential of BP in a mouse model of DPN. We treated wild type C57BL6 mice with the pancreatic beta cell specific toxin streptozotocin (STZ) which leads to beta cell death, impaired insulin production and hyperglycemia in mice (Wu and Huan, 2001). In DPN, sensory nerve damage commonly leads to loss of sensation in the extremities. We evaluated the impact of BP treatment in STZ-treated mice by measuring thermal sensation as a readout of sensory nerve function and by histological analysis of the sciatic nerve to assess structural damage (FIG. 4A). STZ-treated mice showed a dramatic increase in blood glucose levels independent of BP treatment as compared to non-diabetic control animals, indicating that BP treatment does not affect glucose levels. Hyperglycemic mice maintained without BP treatment showed a delayed response to thermal stimulation at seven and eight weeks post-STZ treatment. Remarkably, BP treated diabetic mice showed no significant difference in response time compared to normal, non-diabetic animals (FIG. 4B). Histological analysis revealed a marked increase in the percentage of TUNEL+ apoptotic cells in the sciatic nerves of STZ mice. BP+STZ treated animals showed significantly fewer apoptotic cells than animals with vehicle+STZ treatment (FIG. 4 C and D). Finally, we evaluated the impact of STZ and BP treatment on peripheral myelin using transmission electron microscopy. We observed a large percentage of fibers with damaged myelin in the sciatic nerves of STZ treated animals which was significantly reduced in BP+STZ treated animals (FIG. 4 E and F). These data indicate that BP can partially prevent DPN in STZ treated mice. To assess whether BP is capable of reversing sensory defects and has therapeutic potential in more advanced disease states, we started BP treatment 8 weeks post-STZ in a separate cohort of mice. These mice already showed a delayed response time to thermal stimulation before treatment with BP but showed no significant difference after 4-8 weeks of BP treatment compared to normal mice. These studies demonstrate robust therapeutic effects of BP in the STZ-model of DPN.

Collectively these results highlight the promise of hPSC-derived cultures for modeling SC disorders, understanding disease mechanisms and identifying potential therapeutic strategies.

### Discussion

Our study reports on the highly efficient derivation of SCs from hPSCs which overcomes the limitations of previous studies such as low yield, protracted differentiation, limited SC maturation and lack of myelination data (Huang et al., 2017; Kim et al., 2017; Lee et al., 2007; Liu et al., 2012, 2012; Ziegler et al., 2011). An important feature of our hPSC-based model is the scalability and purity of the resulting SCs and the ability to culture cells for extended periods without losing SC properties. In contrast, primary SCs tend to rapidly lose their properties upon extended culture which results in the increasing contamination of SC cultures with fibroblast-like cells. Important developmental questions that are now accessible using this novel differentiation technology include the mechanisms controlling the transition from a multipotent NC stem cell to committed SCs and the study of human SC plasticity given data in the mouse suggesting that both melanocytes and parasympathetic neurons can be derived from early SC-lineages (Adameyko et al., 2009; Bonnamour et al., 2021; Espinosa-Medina et al., 2014; Nitzan et al., 2013). Our stepwise SC differentiation protocol generates SCs following the developmental processes thought to occur in vivo. It hence provides a powerful framework for lineage tracing studies that would shed light on how NC and SCP fate specification mechanisms lead to the emergence of SCs and other SCPDs such as melanocytes.

Our high-resolution transcriptomics profiling of hPSC-derived SCs is the first study that provides a comprehensive molecular characterization of human SCs at two differentiation stages. In addition to confirming the authenticity of the differentiated Schwann cell types, it allows us to identify specific transcription factors, SC functional markers and surface molecules. Combining these data with high-throughput antibody screening, we identify novel markers for prospective isolation of mature SCs. The identification of CD98 as a surface marker for the prospective isolation of committed SCs represents a powerful tool for such studies. Based on the proof-of-concept data presented here, studies on SC-mediated neuronal maturation and myelination should be other areas of focus. The modeling of PNS pathologies and the development of a drug screening platform for compounds modulating peripheral myelination could be of particular interest. A surprising feature of the cultured hPSC-derived SC is their gene expression pattern that not only confirms SC identity but suggests that pluripotent-derived cells match the expression pattern of adult SC. This is in contrast to most other in vitro derived hPSC-lineages such as neurons expressing fetal stage markers (Studer et al., 2015). Autologous SCs are currently being tested for applications in regenerative medicine targeting both PNS and CNS disorders (Lavdas et al., 2008; Rodrigues et al., 2012). Our transplantation data demonstrate robust engraftment of hPSC-SCs in a model of traumatic nerve injury. While future studies are required to assess long-term engraftment and therapeutic potential in models of PNS and CNS injury, our results set a solid foundation for the application of hPSC-SCs in regenerative medicine including spinal cord injury.

We present an hPSC-based DPN model that revealed a selective vulnerability of SCs to diabetes-associated hyperglycemia. While most of our data were obtained in response to high glucose exposure, we observed decreases in SC viability even at more moderate levels of increased glucose. Through our high-throughput screening, we identified candidate drugs that counteract the glucotoxicity in SCs. Interestingly there are many shared proteins between the predicted targets of these drugs. For validation studies, we focused on our top candidate bupropion. Transcriptional and metabolic profiling of stressed and rescued SCs gave interesting insight into the cellular consequences of SC glucotoxicity and the mechanism of BP rescue. By comparing the list of BP predicted targets and the 66 transcripts that had reversed pattern of expression under glucose and BP treatment, we identified PTGER4 as a potential target that mediates SC glucotoxicity. These proteins convert arachidonate to PGH2 that is the precursor for other prostaglandins including PGD2 and PGF2 and PGE2, the latter being the ligand for PTGER4. In addition, PTGER4 is among the significant targets of our HTS library compound analysis of hits that improve SC viability under high glucose condition. Moreover, prostaglandin analogs have been shown some evidence of moderate efficacy in a randomized clinical trial for DPN (Boulton et al., 2005).

The ability of BP to counteract glucose-mediated SC toxicity correlated with a decrease in intracellular glucose and sucrose levels. BP treatment resulted in reduced cellular lactate concentration and its membrane transporters that might be potentially important given the importance of lactate as a fuel in neuron-SC metabolic coupling (Babetto et al., 2020; Domenech-Estevez et al., 2015). Our transcriptional and metabolomics profiling provide evidence on upregulation of glycolysis and downregulation of mitochondrial respiration in SCs in response to high glucose that were rescued by BP treatment. iPAGE gene set enrichment analysis also identified oxidative phosphorylation as pathways by which HTS hits may mediate their protective effects. Interestingly, BP appears to be the only antidepressant drug commonly associated with moderate weight loss in patients rather than a weight gain (Arterburn et al., 2016). It is tempting to speculate whether BP mediated changes in glucose metabolism could be related to those systemic effects. By performing pathway enrichment analysis, we identified significant changes in glycerolipid metabolism under high glucose condition that was reversed upon BP treatment. This is of significant functional relevance considering the role of lipid metabolism and myelin synthesis in SC function.

Importantly, our in vivo studies demonstrate that BP treatment can rescue DPN-related behavioral deficits and nerve damage. The fact that BP is a widely used drug should greatly facilitate any future testing in DPN patients. Interestingly, BP has shown some benefit in the treatment of patients suffering from neuropathic pain (Semenchuk et al., 2001) raising the question whether those effects of the drug for alternative indications could also be mediated by its effect on SC vulnerability. In addition to BP, we identified several additional compounds capable or rescuing Schwann cell vulnerability. It will be interesting to determine whether those compounds act via a common or distinct mechanism and whether they show in vivo activity in STZ mice comparable to BP.

In conclusion, our findings facilitate human SC-based studies for applications in regenerative medicine and human disease modeling. The work further implicates SC defects in the pathogenesis of DPN and presents BP as an FDA-approved drug that can treat DPN-related damage in vitro and in vivo.

### Materials and Methods

Culture of Human Pluripotent Stem Cells (hPSCs)

hPSC line H9 (WA-09) and derivatives (SOX10::GFP; SYN::ChR2-YFP; SYN::YFP;PHOX2B:GFP;EF1::RFP EDNRB−/−) were maintained on mouse embryonic fibroblasts (MEF, Global Stem, Rockville, MD) in KSR (Life Technologies, 10828-028) containing hPSC medium as described previously (Chambers et al., 2009) or were plated on Geltrex™-coated plates and maintained in chemically-defined medium (E8) as described previously (Barber et al., 2019). Cells were subjected to mycoplasma testing at monthly intervals and STR profiled to confirm cell identity at the initiation of the study.

### Neural Crest Induction

Differentiations of hPSCs towards NC were carried out following previously established methods using the knockout serum (KSR) medium or chemically defined Essential 6 (E6) medium (Fattahi et al., 2016; Tchieu et al., 2017). Briefly, when the monolayer culture of hPSCs reached about 70% confluency, neural crest induction protocol was initiated (D0) by aspirating the maintenance medium (E8) and replacing it with neural crest induction medium A [BMP4 (1 ng ml-1), SB431542 (10 μM), and CHIR 99021 (600 nM) in Essential 6 medium]. Subsequently, on D2 neural crest induction medium B [SB431542 (10 μM) and CHIR 99021 (1.5 μM) in Essential 6 medium] was fed to the cultures until D12. Next, developing precursors are formed during D12-D30 to facilitate the selection for glial progenitor lineages. In doing so, we removed medium B on D12 and detached the NC monolayers using accutase (30 min, 37° C., 5% CO2). After centrifuging the samples at 290×g for 1 min, we re-suspended the cells in NC-C medium [FGF2 (10 ng ml-1), CHIR 99021 (3 μM), N2 supplement (10 μl ml-1), B27 supplement (20 μl ml-1), glutagro (10 μl ml-1), and MEM NEAAs (10 μl ml-1) in neurobasal medium] and transferred them to ultra-low-attachment plates to form free-floating 3D developing precursors. Two days later, when the free-floating developing precursors could be observed, we gently gathered them in the center of each well using a swirling motion. Then, the old media was carefully aspirated from the circumference of each well without removing developing precursors. After addition of the fresh NC-C medium, the cultures were incubated for 48 hours (37° C. and 5% CO2) prior to passaging using accutase. Similarly, cultures were fed with fresh medium every other day and passaged every four days until D30.

Induction and Expansion of Schwann Cells from hPSCs

At day 1, NC cells were aggregated into 3D spheroids (5 million cells/well) in Ultra Low Attachment 6-well culture plates (Fisher Scientific, 3471) and cultured in Neurobasal (NB) medium supplemented with L-Glutamine (Gibco, 25030-164), N2 (Stem Cell Technologies, 07156) and B27 (Life Technologies, 17504044) containing CHIR (3 μM, Tocris Bioscience, 4423) and FGF2 (10 ng/ml, R&D Systems, 233-FB-001MG/CF) and NRG1 (10 ng/ml, R&D 378-SM-025). After 14 days of suspension culture, the spheroids were plated on Poly Omithine/Laminin/Fibronectin (PO/LM/FN) coated dishes (prepared as described previously (17)) in Neurobasal (NB) medium supplemented with L-Glutamine (Gibco, 25030-164), N2 (Stem Cell Technologies, 07156) and B27 (Life Technologies, 17504044) containing NRG1 (20 ng/ml, R&D 378-SM-025), FGF2 (10 ng/ml, R&D Systems, 233-FB-001MG/CF) and cAMP (100 mM, Sigma, D0260). The SC precursors migrate out of the plated spheroids and differentiate into SCs within 10 days. For long-term expansion, cells were cultured in Schwann cell medium (Sciencell, 1701) on PO/LM/FN coated dishes. Cells were fixed for immunostaining or harvested for gene expression analysis at Day 25, Day 35, Day 50 and Day 100 of differentiation.

### FACS and Immunofluorescence (IF) Analysis

For IF, the cells were fixed with 4% paraformaldehyde (PFA, Affymetrix-USB, 19943) for 20 minutes, then blocked and permeabilized using 1% Bovine Serum Albumin (BSA, Thermo Scientific, 23209) and 0.3% triton X-100 (Sigma, T8787). The cells were then incubated in primary antibody solutions overnight at 4° C. (Celsius) and stained with fluorophore conjugated secondary antibodies at RT for 1 hour, the stained cells were then incubated with DAPI (1 ng/ml, Sigma, D9542-5MG) and washed several times before imaging. For Flow Cytometry analysis, the cells are dissociated with Accutase (Innovative Cell Technologies, AT104) and fixed and permeabilized using BD Cytofix/Cytoperm (BD Bioscience, 554722) solution, then washed, blocked and permeabilized using BD Perm/Wash buffer (BD Bioscience, 554723) according to the manufacturer's instructions. The cells are then stained with primary (overnight at 4) and secondary (30 min at room temperature) antibodies and analyzed using a flow Cytometer (FlowJo software). A list of primary antibodies and dilutions is provided in Table S6.

TABLE S6

| Antibody | Source | Cat. Number | Dilution |
|---|---|---|---|
| CASPR | Generous gift from | | |
| | Dr. Joel Black | | |
| CD49D | Biolegend | 304301 | 0.597222222 |
| CHAT | Proteintech | 20747-1-AP | 1:1000 |
| CHAT | Sigma | AB144P | 1:1000 |
| GFAP | Abcam | ab4674 | 1:1000 |
| Kᵥ1.2 | Generous gift from | | |
| | Dr. Joel Black | | |
| MAG | Millipore | LS-C279052-200 | 1:200 |
| MBP | Millipore | MAB386 | 1:200 |
| NFH | Encore | RPCA-NF-H | 1:1000 |
| NFkB p65 | Invitrogen | 710048 | 1:500 |
| PanNa | Generous gift from | | |
| | Dr. Joel Black | | |
| PMP22 | Novus Biologicals | NB110-59086 | 1:100 |
| S100 | Thermo Scientific | RB-9018-P0 | 1:500 |
| SC101 | Takara | Y40400 | 1:1000 |
| TUBB3 | Millipore Sigma | ab9354 | 1:350 |
| TUBB3 | Biolegend | 801202 | 1:1500 |

Single Cell RNA-Sequencing (scRNA-Seq) Data Analysis

### ScRNA-Seq Processing

FASTq files were aligned using the 10× Genomics CellRanger 6.0.0 pipeline (Zheng et al., 2017) to the human GRCh38 reference transcriptome to generate gene-expression counts matrices using the “include introns” option.

### Quality Control and Cell Filtration

Datasets were analyzed in R v4.1.0 with Seurat v4 (Hao et al., 2021). The number of reads mapped to mitochondrial and ribosomal transcripts per cell were derived using the “PercentageFeatureSet” function. We identified cells of poor quality and subsequently removed them independently for each dataset based on the number of unique features captured per cell, the number of unique molecular identifiers (UMI) captured per cell and the percentage of mitochondrial gene transcripts per cell. Datasets were filtered based on the following quality control metrics: nFeatures>200, nFeatures<7000, nCounts<40000 and percent mitochondrial reads <20%.

### Dimensionality Reduction, Clustering and Annotation

Transcript count matrices were log normalized applying a scaling factor of 10,000 with 2,000 variable features identified using the “vst” method. Cell cycle phase distribution was predicted using the “CellCycleScoring” function with Seurat's S and G2M features available in “cc.genes”. The 2000 most variable feature set was scaled and centered, and the following data variables were regressed out: nFeatures, nCounts, mitochondrial gene percentage, ribosomal gene percentage, S score and G2M score. Principal Components Analysis (PCA) was run using default settings. PCA reduction was used to perform Uniform Manifold Approximation and Projection (UMAP) dimensionality reduction. The shared nearest neighbors (SNN) graph was computed using default settings followed by cell clustering achieved using the default Louvain algorithm. Quality control metrics were visualized per cluster to identify and remove clusters of low-quality cells (see Quality Control and Cell Filtration). The above pipeline was performed again on each dataset after the removal of low-quality cell clusters. The number of principal components used for UMAP reduction and SNN calculation was determined by principal component standard deviation unique for each dataset together with resolution used for clustering of each dataset can be found in Table S7. Cluster markers were derived using the Wilcoxon Rank Sum test. Cluster annotation was based on the expression of known cell type marker genes. Following cell type annotation, gene dropout values were imputed using adaptively-thresholded low rank approximation (ALRA) (Linderman et al., 2018). The rank-k approximation was automatically chosen for each dataset with default values selected for all other parameters. The imputed gene expression is depicted in all plots and used as default in all downstream analyses unless otherwise specified.

TABLE S7

| | | | # PCs | | | | | | Cluster(s) |
|---|---|---|---|---|---|---|---|---|---|
| | Cell | Batch | Used | | | | | | removed and re- |
| | Type | Correction | (SNN and | UMAP | UMAP | UMAP | UMAP | Clustering | clustered? |
| Dataset | Subset | Method | UMAP) | min.dist | n.neighbors | metric | umap.method | Resolution | (Reason) |
| Low | All | NA | 46 | 0.3 | 46 | cosine | uwot | 0.15 | Yes |
| passage | cells | (regular | | | | | | | (contaminating |
| SCs | | merge) | | | | | | | cell type |
| | | | | | | | | | specific marker |
| | | | | | | | | | expression) |
| High | All | NA | 35 | 0.3 | 35 | cosine | uwot | 0.35 | Yes |
| passage | cells | (regular | | | | | | | (contaminating |
| SCs | | merge) | | | | | | | cell type |
| | | | | | | | | | specific marker |
| | | | | | | | | | expression) |

### Analysis of Published Datasets

Primary tissue derived Schwann cell type markers for Schwann cell precursor, myelinating and non-myelinating Schwann cells were obtained from Tasdemir-Yilmaz et. al. (Tasdemir-Yilmaz et al., 2021) using interactive webpage Pagoda 2 by performing differential feature expression analysis of cell type cluster of interest against the entirety of dataset cells. Differentially expressed (DE) genes were sorted by Z-score and converted to human gene names with “biomaRt” (Durinck et al., 2009) package using human and mouse genome databases available at ensembl.

### Gene Group Expression Characterization

Gene lists were compiled for genes belonging to transcription factor, surface marker, cell adhesion, neurotransmitter receptor and neurotrophic factor functional groups from Molecular Signatures Database (MSigDB) (Liberzon et al., 2011). For each dataset, the gene lists were filtered to remove low abundance genes (detected in less than 25% of cells of each cell type cluster). Genes were then determined to be exclusively expressed by a cluster if greater than 25% of cells within that cluster only expressed the gene.

To further selectively filter transcription factor and surface marker gene sets we derived genes shared by transcription factor and surface marker gene sets and cell type specific differentially expressed (DE) gene lists.

### Cell Type Transcriptional Signature Scoring

To find transcriptionally similar cell populations between two datasets, first the differentially expressed (DE) genes of the reference dataset were calculated from the imputed gene counts with the “FindAllMarkers” function using the Wilcoxon Rank Sum test and only genes with a fold change (FC) above 0.25 were returned. The reference DE gene lists were filtered to remove genes not present in the query dataset. Then for each cell cluster in the reference dataset, a transcriptional signature gene list was made from the top 100 DE genes filtered for p value below 0.05 and sorted by decreasing fold change (FC). The query dataset is then scored for the transcriptional signature gene lists of each reference dataset cell cluster using the “AddModuleScore” function based on the query dataset's imputed feature counts.

### Myelinating Schwann Cell Identity Specification

Myelinating Schwann cell (mySC) specific marker gene set was curated by combining published dataset derived markers (Jessen and Mirsky, 2005) and canonical myelination associated markers for a total of 21 marker genes (Table S3). LP and HP specific mature Schwann cell clusters were subset from entire LP and HP datasets and scored for mySC gene set using the “AddModuleScore” function. Cells with positive mySC score were isolated and identified as mySC in further analyses.

### Gene Ontology Analysis

Cell type of interest specific DE genes with positive fold change (FC) were calculated from the imputed gene counts with the “FindAllMarkers” function. Each gene set was filtered to include genes with p value<0.05 and sorted by decreasing fold change. Where possible up to 250 genes from each cell type specific dataset were used in gene functional profiling analysis by using g:Profiler (Raudvere et al., 2019) online tool and selecting pathways from GO biological process, KEGG and Reactome databases. Term enrichment was ranked by decreasing value of negative log 10 transformed p values.

### Surface Marker Screening

Screening for specific surface antigens was performed using BD Lyoplate Library® (BD, 560747) on hPSC-SCs at day 80 of differentiation. Cells were plated in 96 well plates (10,000 cells/well) and stained with primary and secondary antibodies according to manufacturer's instructions. The stained wells were fixed for total plate imaging and quantification. The percentage of double positive cells out of total GFAP was quantified for each antibody. Top hits (>60% double positive) were validated further using flow cytometry.

### Gene Expression Analysis

For RNA sequencing, total RNA was extracted using RNeasy RNA purification kit (Qiagen, 74106). For qRT-PCR assay, total RNA samples were reverse transcribed to cDNA using Superscript II Reverse Transcriptase (Life Technologies, 18064-014). qRT-PCR reactions were set up using QuantiTect SYBR Green PCR mix (Qiagen, 204148). Each data point represents three independent biological replicates. RNA-seq reads were mapped to the human reference genome (hg19) using TopHat v2.0. TopHat was run with default parameters with exception to the coverage search. Alignments were then quantified using HTSeq and differential gene expression was calculated using DESeq normalized to the cranial neural crest sample.

### Viability Assay

To monitor the viability of SCs, cells were assayed for LDH activity using CytoTox 96 cytotoxicity assay kit (Promega, G1780). Briefly, the cells are plated in 96 well plates at 30,000 cells/cm2. The supernatant and the cell lysate is harvested 24 hours later and assayed for LDH activity using a plate reader (490 nm absorbance). Cytotoxicity is calculated by dividing the LDH signal of the supernatant by total LDH signal (from lysate plus supernatant). The cells were cultured in Schwann cell medium (Sciencell, 1701) on PO/LM/FN coated dishes during the assay.

### Calcium Imaging

MN-only cultures and MN-SC co-cultures were subjected to calcium imaging at days 40 and 70 post-co-culture as previously described (Barreto-Chang and Dolmetsch, 2009). Briefly, The cells were loaded with 2 μmol/L Fluo-4 AM dissolved in 1:1 (v/v) amount of 20% Pluronic®-F127 and DMSO with stock concentration of 1 mmol/L for 45 min at RT in Tyrode solution consisting of (mmol/L): 140 NaCl, 5.4 KCl, 1 MgCl2, 1.8 CaCl2, 10 glucose and 10 HEPES at pH 7.4. For activation, cells were spiked with a solution containing glutamate (50 mM) or KCl (300 mM). Time lapse images were acquired using an Axiovert Inverted Microscope (Zeiss) on a heated stage. Ratiometric analysis was performed using Metamorph Software (Molecular Devices).

Transplantation of hPSC-SCs in Rat Sciatic Nerves and Histological Assessment

All procedures were performed following NIH guidelines, and were approved by the local Institutional Animal Care and Use Committee (IACUC). Rats were placed under isoflurane gas anesthesia and both sciatic nerves were exposed below the sciatic notch and crushed using Dumont #5 forceps for 30 seconds twice in the same location. Immediately afterwards, a cell suspension of 3×10⁴ hPSCs/μl Schwann cells were transplanted via injection of ˜3-4 μl at proximal and distal locations to the crush site with a glass micropipette. Survival times ranged from 2 to 8 weeks. For immunohistochemistry, tissue was fixed through intracardiac perfusion of 4% PFA in 0.1 M PBS. Sciatic nerves were dissected from rats at 2, 3, 4, 8 weeks after crush lesion and transplantation. After dissection, sciatic nerves where prepared by placing them in 30% sucrose in 0.1 M PBS overnight and embedding them in OCT blocks for cryosectioning, or, by removing the perineurium and teasing them in cold 0.1 M Phosphate Buffer (pH 7.4). Some nerves were teased after perfusion and immunostained to examine individual axons. Regenerated axons distal to the crush site were analyzed.

### Metabolite Measurements

High glucose, low glucose and drug treated SCs and sensory neurons were subjected to biochemical assays for Sorbitol (Abcam, ab118968), glucose (Abcam, ab65333), Pyruvate (Abcam, ab65342) and 2DG uptake (Abcam, ab136955). The measurements were carried out according to the manufacturer's instructions. Data were normalized according to cell numbers and averaged across 3-6 biological replicates.

High-throughput screening assay for drugs reversing glucose-mediated SC cytotoxicity The chemical compound screening was performed using the Prestwick Chemical Library®. RFP-labeled hPSC-SCs were plated in 384 well plates (1,000 cells/well) and treated with 30 mM glucose immediately before addition of the compounds. The compounds were added at 1 μM concentration. After 72 hours, the plates were treated with DAPI for ten minutes, washed twice and fixed for total plate imaging. The number of viable cells was quantified for each well by counting the number of DAPI negative, RFP positive cells. For validation of the selected hit compound (Bupropion HCL, Sigma, B102), the cells were treated with various concentrations of the compound for dose response analysis. The highest non-toxic dose (0.7 μM; based on sorbitol reduction and viability) was used for follow-up experiments.

### Drug Target Prediction

Z-scores for primary hit compounds were calculated as Z=(x−μ)/σ. X is the number of viable cells. μ is the mean number of viable cells and σ is the standard deviation for all compounds and DMSO controls. The normalized z-score values reported for all the compounds were first transformed to N(0,1) using the bestNormalize package (v1.4.0) in R (v3.5.1). The treatments with transformed z-scores greater than 2 were selected, which resulted in 16 hit compounds.

To identify the proteins that are most likely targeted by the SC protecting drugs against glucotoxicity, we employed two independent tests. First, we calculated combined z-score in which we integrated normalized z-score from all of the treatments associated with a particular protein. Second, we performed a Fisher's exact test to see if a target protein is enriched among the targets of treatments with positive z-scores. We report the correlation between the p-values calculated by these two independent tests. For every compound, possible target proteins were identified as above. Weighted combined z-scores were then calculated for each protein by combining normalized z-scores across all treatments (Zaykin, 2011). The p-values were then calculated based on the combined z-scores and adjusted using p.adjust (method=FDR). As an orthogonal approach, for each protein, we recorded the number of treatments with positive normalized z-scores as well as the total number of compounds predicted to target that protein. Using the sum of counts for all other proteins and drugs, we performed a Fisher's exact test to evaluate the degree to which positive z-scores were enrichment among the treatments likely to affect a protein of interest. As expected, the two p-values, i.e. combined z-score and Fisher's, are generally correlated.

### Protein-Protein Interaction Network Construction.

Protein-protein interaction network analysis was performed using the Search Tool for the Retrieval of Interacting Genes (STRING) database. The minimum required interaction score was set to 0.4 corresponding to medium confidence. The edge thickness indicates the degree of data support from the following active interaction sources: textmining, experiments, databases, co-expression, neighborhood, gene fusion and co-occurrence.

iPAGE Pathway Enrichment Analysis

The iPAGE algorithm was used for gene set and pathway enrichment analysis (Goodarzi et al., 2009). iPAGE first quantize continuous input data into equally populated bins and then, it calculates the Mutual Information (MI) between a vector of values for genes within each cluster bin and a binary vector of gene set memberships. The significance of the calculated MI values is then assessed through a randomization-based statistical test. Finally, it uses hypergeometric distribution to determine the level with which the significantly informative pathways are overrepresented (red) or underrepresented (blue) in each cluster bin. The resulting p-values asses to draw heatmap visualization, in which rows represent significant pathways and columns correspond to cluster bins. We first ordered target genes identified from FDA approved drug library screening with positive z-score (see drug target prediction above) based on their combined z-score from left to right and followed by dividing them into seven equally populated bins. We then assessed the enrichment (red boxes) and depletion (blue boxes) of various gene sets across the spectrum (MSigDB v6.0). Gene sets provided were from the Molecular Signatures Database (MSigDB v6.0) database (Liberzon et al., 2015). Here, we report enriched gene sets from cluster 2 (C2, curated gene sets), and cluster 5 (C5, ontology gene sets).

### NF-kB Staining

Image analysis was performed using FIJI (Schindelin et al., 2012). Watershedding was applied to separate nuclei that were grouped together. Nuclear ROIs were overlayed onto the NF-KB image to calculate nuclear co-localization. To analyze NF-KB in each condition, several measures were taken. Because the cytoplasm could not be isolated to represent the cell to which it belongs and because the average of the mean intensity of the cytoplasmic ROIs would not account for the distribution of intensities per ROI area, the integrated densities of the cytoplasmic ROIs were summed together and divided by the sum of the cytoplasmic ROIs' area to give the mean intensity of NF-KB across all cytoplasms in each image. The mean intensities of each nuclei were divided by the mean intensity of the cytoplasm to give a ratio of nuclear to cytoplasmic NF-KB expression.

### Metabolomics

hPSC-Scs were treated with 5 mM and 30 mM glucose for 72 hours and harvested for metabolomics analysis. Frozen total cell pellets from at least three biological repeats were submitted to the West Coast Metabolomics Center at the University of California, Davis that used Agilent 6890 gas chromatographer and Pegasus III TOF mass spectrometer for an untargeted primary metabolomics analysis. Metaboanalyst (Xia et al., 2009) was used to generate the heat maps and perform path analysis.

### Fructose, Lactate and Sorbitol Measurements

Cellular levels of fructose, lactate and sorbitol were measured by colorimetric assays following the instructions provided by the manufacturers. The following kits were used: fructose (Abeam, ab83380), lactate (Abeam, ab65331) and sorbitol (Abeam, ab118968)

### CRISPR Knockout of PTGER4

Schwann PTGER4 ribonucleoprotein (RNP) complex was assembled by mixing 180 pmol of multiguide sgRNA (Synthego, USA) and 20 pmol of Cas9 2NLS (Berkeley QB3) in Lonza electroporation buffer P3 (Lonza, Switzerland) per reaction. hPSC-derived Schwann cells were detached using trypsin and washed 2× with PBS. 250,000 cells per reaction were resuspended in Lonza electroporation buffer P3 immediately before electroporation. Cells were mixed with the RNPs and were electroporated using a Lonza 4D 96 well electroporation system with pulse code DS-137. Ten minutes post nucleofection, cells were then diluted in warm medium and were plated. Medium was changed on the following day, cells were passed as needed until the day of the assay. For quantification, images were analyzed with NIH ImageJ software by measuring the fluorescence intensity of individual cells by manual region of interest selection.

### Drug Treatment of Diabetic Mice

All procedures were performed following NIH guidelines, and were approved by the local Institutional Animal Care and Use Committee (IACUC). 3-8 weeks old male C57BL6 mice were treated with one dose IP injection of STZ (180 mg/kg, sigma, 85882) to induce pancreatic beta cell death. Blood glucose levels were measured using a standard Glucometer (Freestyle Lite) in weekly intervals starting at one week post-treatment, by drawing a drop of blood from tail tip. BP treatment was initiated one week post-STZ treatment. BP was mixed with standard chow at 1.63 mg/g of chow to be administered orally at ˜300 mg/kg daily. The dose was calculated based on average daily food intake (5.5 g/day) and initial body weight (30 g).

### Mouse Thermal Sensitivity Test

Thermal nociception was assessed using the hot plate test. The hot plate (Ugo Basile 35100) consisted of a metal surface (55° C.) with a transparent Plexiglas cylinder to contain the mouse. The subject is placed upon a constant temperature hot plate and the latency necessary to demonstrate discomfort, assessed by either licking/shaking the hind paw or jumping, is determined. Typical baseline latencies are 5-10 seconds, with maximal latencies of 30 seconds. Any animal that does not demonstrate discomfort behavior will be removed after 30 seconds, the maximal latency, to avoid tissue damage.

### Statistical Analysis

Data are presented as mean±SEM and were derived from at least 3 independent experiments. Data on replicates (n) is shown in figures. Statistical analysis was performed using the Student t-test (comparing 2 groups) or ANOVA with Dunnett test (comparing multiple groups against control). Distribution of the raw data approximated normal distribution (Kolmogorov Smirnov normality test) for data with sufficient number of replicates to test for normality.

### REFERENCES

Adameyko, I., Lallemend, F., Aquino, J. B., Pereira, J. A., Topilko, P., Müller, T., Fritz, N., Beljajeva, A., Mochii, M., Liste, I., et al. (2009). Schwann Cell Precursors from Nerve Innervation Are a Cellular Origin of Melanocytes in Skin. Cell 139, 366-379.
Arterburn, D., Sofer, T., Boudreau, D. M., Bogart, A., Westbrook, E. O., Theis, M. K., Simon, G., and Haneuse, S. (2016). Long-Term Weight Change after Initiating Second-Generation Antidepressants. J. Clin. Med. 5, E48.
Babetto, E., Wong, K. M., and Beirowski, B. (2020). A glycolytic shift in Schwann cells supports injured axons. Nat. Neurosci. 23, 1215-1228.
Bajpai, R., Chen, D. A., Rada-Iglesias, A., Zhang, J., Xiong, Y., Helms, J., Chang, C.-P., Zhao, Y., Swigut, T., and Wysocka, J. (2010). CHD7 cooperates with PBAF to control multipotent neural crest formation. Nature 463, 958-962.
Barber, K., Studer, L., and Fattahi, F. (2019). Derivation of enteric neuron lineages from human pluripotent stem cells. Nat. Protoc. 14, 1261-1279.
Barreto-Chang, O. L., and Dolmetsch, R. E. (2009). Calcium imaging of cortical neurons using Fura-2 AM. J. Vis. Exp. JoVE 1067.
Bonnamour, G., Soret, R., and Pilon, N. (2021). Dhh-expressing Schwann cell precursors contribute to skin and cochlear melanocytes, but not to vestibular melanocytes. Pigment Cell Melanoma Res. 34, 648-654.
Boulton, A. J. M., Vinik, A. I., Arezzo, J. C., Bril, V., Feldman, E. L., Freeman, R., Malik, R. A., Maser, R. E., Sosenko, J. M., Ziegler, D., et al. (2005). Diabetic neuropathies: a statement by the American Diabetes Association. Diabetes Care 28, 956-962.
Calder, E. L., Tchieu, J., Steinbeck, J. A., Tu, E., Keros, S., Ying, S.-W., Jaiswal, M. K., Cornacchia, D., Goldstein, P. A., Tabar, V., et al. (2015). Retinoic Acid-Mediated Regulation of GLI3 Enables Efficient Motoneuron Derivation from Human ESCs in the Absence of Extrinsic SHH Activation. J. Neurosci. Off. J. Soc. Neurosci. 35, 11462-11481.
Callaghan, B. C., Cheng, H. T., Stables, C. L., Smith, A. L., and Feldman, E. L. (2012). Diabetic neuropathy: clinical manifestations and current treatments. Lancet Neurol. 11, 521-534.
Chambers, S. M., Fasano, C. A., Papapetrou, E. P., Tomishima, M., Sadelain, M., and Studer, L. (2009). Highly efficient neural conversion of human ES and iPS cells by dual inhibition of SMAD signaling. Nat. Biotechnol. 27, 275-280.
Chambers, S. M., Qi, Y., Mica, Y., Lee, G., Zhang, X.-J., Niu, L., Bilsland, J., Cao, L., Stevens, E., Whiting, P., et al. (2012). Combined small-molecule inhibition accelerates developmental timing and converts human pluripotent stem cells into nociceptors. Nat. Biotechnol. 30, 715-720.
Corrêa-Silva, S., Alencar. A. P., Moreli, J. B., Borbely, A. U., de S Lima, L., Scavone, C., Damasceno, D. C., Rudge, M. V. C., Bevilacqua, E., and Calderon, I. M. P. (2018). Hyperglycemia induces inflammatory mediators in the human chorionic villous. Cytokine 111, 41-48.
Domènech-Estévez, E., Baloui, H., Repond, C., Rosafio, K., Médard, J.-J., Tricaud, N., Pellerin, L., and Chrast, R. (2015). Distribution of monocarboxylate transporters in the peripheral nervous system suggests putative roles in lactate shuttling and myelination. J. Neurosci. Off. J. Soc. Neurosci. 35, 4151-4156.
Durinck, S., Spellman, P. T., Birney, E., and Huber, W. (2009). Mapping identifiers for the integration of genomic datasets with the R/Bioconductor package biomaRt. Nat. Protoc. 4, 1184-1191.
Eckersley, L. (2002). Role of the Schwann cell in diabetic neuropathy. Int. Rev. Neurobiol. 50, 293-321.
Espinosa-Medina, I., Outin, E., Picard, C. A., Chettouh, Z., Dymecki, S., Consalez, G. G., Coppola, E.,
and Brunet, J.-F. (2014). Neurodevelopment. Parasympathetic ganglia derive from Schwann cell precursors. Science 345, 87-90.
Fattahi, F., Steinbeck, J. A., Kriks, S., Tchieu, J., Zimmer, B., Kishinevsky, S., Zeltner, N., Mica, Y., El-Nachef, W., Zhao, H., et al. (2016). Deriving human ENS lineages for cell therapy and drug discovery in Hirschsprung disease. Nature 531, 105-109.
Finzsch, M., Schreiner, S., Kichko, T., Reeh, P., Tann, E. R., Bösl, M. R., Meijer, D., and Wegner, M.
(2010). Sox10 is required for Schwann cell identity and progression beyond the immature Schwann cell stage. J. Cell Biol. 189, 701-712.
Gfeller, D., Grosdidier, A., Wirth, M., Daina, A., Michielin, O., and Zoete, V. (2014). SwissTargetPrediction: a web server for target prediction of bioactive small molecules. Nucleic Acids Res. 42, W32-38.
Goodarzi, H., Elemento, O., and Tavazoie, S. (2009). Revealing global regulatory perturbations across human cancers. Mol. Cell 36, 900-911.
Hao, W., Tashiro, S., Hasegawa, T., Sato, Y., Kobayashi, T., Tando, T., Katsuyama, E., Fujie, A., Watanabe, R., Morita, M., et al. (2015). Hyperglycemia Promotes Schwann Cell Dedifferentiation and De-myelination via Sorbitol Accumulation and Igf1 Protein Down-regulation. J. Biol. Chem. 290, 17106-17115.
Hao, Y., Hao, S., Andersen-Nissen, E., Mauck, W. M., Zheng, S., Butler, A., Lee, M. J., Wilk, A. J., Darby, C., Zager, M., et al. (2021). Integrated analysis of multimodal single-cell data. Cell 184, 3573-3587.e29.
Huang, C.-W., Huang, W.-C., Qiu, X., Fernandes Ferreira da Silva, F., Wang, A., Patel, S., Nesti, L. J., Poo, M.-M., and Li, S. (2017). The Differentiation Stage of Transplanted Stem Cells Modulates Nerve Regeneration. Sci. Rep. 7, 17401.
Huang, D. W., Sherman, B. T., and Lempicki, R. A. (2009a). Bioinformatics enrichment tools: paths toward the comprehensive functional analysis of large gene lists. Nucleic Acids Res. 37, 1-13.
Huang, D. W., Sherman, B. T., and Lempicki, R. A. (2009b). Systematic and integrative analysis of large gene lists using DAVID bioinformatics resources. Nat. Protoc. 4, 44-57.
Jessen, K. R., and Mirsky, R. (2005). The origin and development of glial cells in peripheral nerves. Nat. Rev. Neurosci. 6, 671-682.
Jessen, K. R., Mirsky, R., and Lloyd, A. C. (2015). Schwann Cells: Development and Role in Nerve Repair. Cold Spring Harb. Perspect. Biol. 7, a020487.
Keiser, M. J., Roth, B. L., Armbruster, B. N., Emsberger, P., Irwin, J. J., and Shoichet, B. K. (2007). Relating protein pharmacology by ligand chemistry. Nat. Biotechnol. 25, 197-206.
Kim, H.-S., Lee, J., Lee, D. Y., Kim, Y.-D., Kim, J. Y., Lim, H. J., Lim, S., and Cho, Y. S. (2017). Schwann Cell Precursors from Human Pluripotent Stem Cells as a Potential Therapeutic Target for Myelin Repair. Stem Cell Rep. 8, 1714-1726.
Kim, S., Chen, J., Cheng, T., Gindulyte, A., He, J., He, S., Li, Q., Shoemaker, B. A., Thiessen, P. A., Yu, B., et al. (2019). PubChem 2019 update: improved access to chemical data. Nucleic Acids Res. 47, D1102-D1109.
Kowluru, R. A., Koppolu, P., Chakrabarti, S., and Chen, S. (2003). Diabetes-induced activation of nuclear transcriptional factor in the retina, and its inhibition by antioxidants. Free Radic. Res. 37, 1169-1180.
Lavdas, A. A., Papastefanaki, F., Thomaidou, D., and Matsas, R. (2008). Schwann cell transplantation for CNS repair. Curr. Med. Chem. 15, 151-160.
Lee, G., Kim, H., Elkabetz, Y., Al Shamy, G., Panagiotakos, G., Barberi, T., Tabar, V., and Studer, L. (2007). Isolation and directed differentiation of neural crest stem cells derived from human embryonic stem cells. Nat. Biotechnol. 25, 1468-1475.
Liberzon, A., Subramanian, A., Pinchback, R., Thorvaldsdóttir, H., Tamayo, P., and Mesirov, J. P. (2011). Molecular signatures database (MSigDB) 3.0. Bioinforma. Oxf. Engl. 27, 1739-1740.
Liberzon, A., Birger, C., Thorvaldsdóttir, H., Ghandi, M., Mesirov, J. P., and Tamayo, P. (2015). The Molecular Signatures Database (MSigDB) hallmark gene set collection. Cell Syst. 1, 417-425.
Linderman, G. C., Zhao, J., and Kluger, Y. (2018). Zero-preserving imputation of scRNA-seq data using low-rank approximation.
Liu, Q., Spusta, S. C., Mi, R., Lassiter, R. N. T., Stark, M. R., Höke, A., Rao, M. S., and Zeng, X. (2012). Human neural crest stem cells derived from human ESCs and induced pluripotent stem cells: induction, maintenance, and differentiation into functional schwann cells. Stem Cells Transl. Med. 1, 266-278.
Liu, T., Lin, Y., Wen, X., Jorissen, R. N., and Gilson, M. K. (2007). BindingDB: a web-accessible database of experimentally determined protein-ligand binding affinities. Nucleic Acids Res. 35, D198-201.
Maekawa, K., Tanimoto, T., Okada, S., Suzuki, T., Suzuki, T., and Yabe-Nishimura, C. (2001). Expression of aldose reductase and sorbitol dehydrogenase genes in Schwann cells isolated from rat: effects of high glucose and osmotic stress. Brain Res. Mol. Brain Res. 87, 251-256.
Mathias, S. L., Hines-Kay, J., Yang, J. J., Zahoransky-Kohalmi, G., Bologa, C. G., Ursu, O., and Oprea, T. I. (2013). The CARLSBAD database: a confederated database of chemical bioactivities. Database J. Biol. Databases Curation 2013, bat044.
Menendez, L., Yatskievych, T. A., Antin, P. B., and Dalton, S. (2011). Wnt signaling and a Smad pathway blockade direct the differentiation of human pluripotent stem cells to multipotent neural crest cells. Proc. Natl. Acad. Sci. 108, 19240-19245.
Mica, Y., Lee, G., Chambers, S. M., Tomishima, M. J., and Studer, L. (2013). Modeling neural crest induction, melanocyte specification, and disease-related pigmentation defects in hESCs and patient-specific iPSCs. Cell Rep. 3, 1140-1152.
Mizisin, A. P. (2014). Mechanisms of diabetic neuropathy: Schwann cells. Handb. Clin. Neurol. 126, 401-428.
Mizisin, A. P., and Powell, H. C. (1993). Schwann cell injury is attenuated by aldose reductase inhibition in galactose intoxication. J. Neuropathol. Exp. Neurol. 52, 78-86.
Newbern, J., and Birchmeier, C. (2010). Nrg1/ErbB signaling networks in Schwann cell development and myelination. Semin. Cell Dev. Biol. 21, 922-928.
Nitzan, E., Pfaltzgraff, E. R., Labosky, P. A., and Kalcheim, C. (2013). Neural crest and Schwann cell progenitor-derived melanocytes are two spatially segregated populations similarly regulated by Foxd3. Proc. Natl. Acad. Sci. U.S.A 110, 12709-12714.
Oates, P. J. (2002). Polyol pathway and diabetic peripheral neuropathy. Int. Rev. Neurobiol. 50, 325-392.
Raudvere, U., Kolberg, L., Kuzmin, I., Arak, T., Adler, P., Peterson, H., and Vilo, J. (2019). g:Profiler: a web server for functional enrichment analysis and conversions of gene lists (2019 update). Nucleic Acids Res. 47, W191-W198.
Rodrigues, M. C. O., Rodrigues, A. A., Glover, L. E., Voltarelli, J., and Borlongan, C. V. (2012). Peripheral Nerve Repair with Cultured Schwann Cells: Getting Closer to the Clinics. Sci. World J. 2012, e413091.
Samuel, R. M., Majd, H., Richter, M. N., Ghazizadeh, Z., Zekavat, S. M., Navickas, A., Ramirez, J. T., Asgharian, H., Simoncau, C. R., Bonser, L. R., et al. (2020). Androgen Signaling Regulates SARS-CoV-2 Receptor Levels and Is Associated with Severe COVID-19 Symptoms in Men. Cell Stem Cell 27, 876-889.e12.
Schindelin, J., Arganda-Carreras, I., Frise, E., Kaynig, V., Longair, M., Pietzsch, T., Preibisch, S., Rueden, C., Saalfeld, S., Schmid, B., et al. (2012). Fiji: an open-source platform for biological-image analysis. Nat. Methods 9, 676-682.
Semenchuk, M. R., Sherman, S., and Davis, B. (2001). Double-blind, randomized trial of bupropion SR for the treatment of neuropathic pain. Neurology 57, 1583-1588.
Simmons, Z., and Feldman, E. L. (2002). Update on diabetic neuropathy. Curr. Opin. Neurol. 15, 595-603.
Siramshetty, V. B., Eckert, O. A., Gohlke, B.-O., Goede, A., Chen, Q., Devarakonda, P., Preissner, S., and Preissner, R. (2018). SuperDRUG2: a one stop resource for approved/marketed drugs. Nucleic Acids Res. 46, D1137-D1143.
Studer, L., Vera, E., and Cornacchia, D. (2015). Programming and Reprogramming Cellular Age in the Era of Induced Pluripotency. Cell Stem Cell 16, 591-600.
Szklarczyk, D., Gable, A. L., Lyon, D., Junge, A., Wyder, S., Huerta-Cepas, J., Simonovic, M., Doncheva, N. T., Morris, J. H., Bork, P., et al. (2019). STRING v11: protein-protein association networks with increased coverage, supporting functional discovery in genome-wide experimental datasets. Nucleic Acids Res. 47, D607-D613.
Tang, X., Zhou, L., Wagner, A. M., Marchetto, M. C. N., Muotri, A. R., Gage, F. H., and Chen, G. (2013). Astroglial cells regulate the developmental timeline of human neurons differentiated from induced pluripotent stem cells. Stem Cell Res. 11, 743-757.
Tasdemir-Yilmaz, O. E., Druckenbrod, N. R., Olukoya, O. O., Dong, W., Yung, A. R., Bastille, I., Pazyra-Murphy, M. F., Sitko, A. A., Hale, E. B., Vigneau, S., et al. (2021). Diversity of developing peripheral glia revealed by single-cell RNA sequencing. Dev. Cell 56, 2516-2535.e8.
Tchieu, J., Zimmer, B., Fattahi, F., Amin, S., Zeltner, N., Chen, S., and Studer, L. (2017). A Modular Platform for Differentiation of Human PSCs into All Major Ectodermal Lineages. Cell Stem Cell 21, 399-410.e7.
Wu, K. K., and Huan, Y. (2001). Streptozotocin-Induced Diabetic Models in Mice and Rats. In Current Protocols in Pharmacology, (John Wiley & Sons, Inc.), p.
Xia, J., Psychogios, N., Young, N., and Wishart, D. S. (2009). MetaboAnalyst: a web server for metabolomic data analysis and interpretation. Nucleic Acids Res. 37, W652-660.
Yamanishi, Y., Kotera, M., Moriya, Y., Sawada, R., Kanehisa, M., and Goto, S. (2014). DINIES: drug-target interaction network inference engine based on supervised analysis. Nucleic Acids Res. 42, W39-45.
Zaykin, D. V. (2011). Optimally weighted Z-test is a powerful method for combining probabilities in meta-analysis. J. Evol. Biol. 24, 1836-1841.
Zheng, G. X. Y., Terry, J. M., Belgrader, P., Ryvkin, P., Bent, Z. W., Wilson, R., Ziraldo, S. B., Wheeler, T. D., McDermott, G. P., Zhu, J., et al. (2017). Massively parallel digital transcriptional profiling of single cells. Nat. Commun. 8, 14049.
Ziegler, L., Grigoryan, S., Yang, I. H., Thakor, N. V., and Goldstein, R. S. (2011). Efficient generation of schwann cells from human embryonic stem cell-derived neurospheres. Stem Cell Rev. 7, 394-403.

### Example 2

Culture Methods in Differentiation and Culture of hPSCs into Neural Crest Cells and Schwann Cells.
Culture of Human Pluripotent Stem Cells (hPSCs)

hPSC line H9 (WA-09) and derivatives (SOX10::GFP; EF1::RFP were maintained on mouse embryonic fibroblasts (MEF, Global Stem, Rockville, MD) in KSR (Life Technologies, 10828-028) containing hPSC medium as described previously or were plated on gel-Trex™-coated (Thermo Fisher Scientific, A1413302) plates and maintained in chemically-defined Essential 8 (E8) medium as described previously.10 WTC11 cells were also maintained in E8 medium.

### Neural Crest (NC) and Schwann Cell (SC) Differentiation

To induce neural crest-derived Schwann cells we used two different methods. In option 1, we performed neural crest induction using KnockOut serum replacement (KSR, Life Technologies, 10828028) containing media and SC induction media supplemented with NRG1 (R&D 378-SM-025). In option 2, we used Essential 6 (E6, Life Technologies, A1516401) medium for neural crest induction and SC induction media without additional NRG1 supplement.

### NC Induction Option 1

hESCs were plated on Matrigel (BD Biosciences, 354234) coated dishes (10⁵ cells/cm2) in hESC medium containing 10 ng/ml FGF2 (R&D Systems, 233-FB-001MG/CF). NC differentiation was initiated in knockout serum replacement (KSR) medium (KO DMEM+15% KSR, L-glutamine (Life Technologies, 25030-081), NEAA (Life Technologies, 11140-050) containing LDN193189 (100 nM, Stemgent, Cambridge, MA) and SB431542 (10 mM, Tocris, Ellisville, MI). For NC induction, cells are treated with 3 mM CHIR99021 (Tocris Bioscience, 4423) in addition to LDN and SB from day 2 through day 11. The KSR medium was gradually replaced with increasing amounts of N2 medium from day 4 through day 10 as described previously58. The differentiated cells are sorted for CD49D at day 11. CNS precursor control cells were generated by treatment with LDN and SB from day 0 through day 11 as previously described [58—Chambers, S. M., Fasano, C. A., Papapetrou, E. P., Tomishima, M., Sadelain, M., and Studer, L. (2009). Highly efficient neural conversion of human ES and iPS cells by dual inhibition of SMAD signaling. Nat. Biotechnol. 27, 275-280, which is incorporated by reference in its entirety]. Throughout the disclosure, day 0 is the day the medium is switched from hESC medium to differentiation medium. Days of differentiation in text and figures refer to the number of days since the pluripotent stage (day 0).

### Induction and Expansion of Schwann Cells Option 1

At day 11, NC cells were aggregated into 3D spheroids (5 million cells/well) in Ultra Low Attachment 6-well culture plates (Fisher Scientific, 3471) and cultured in Neurobasal (NB) medium supplemented with L-Glutamine (Gibco, 25030-164), N2 (Stem Cell Technologies, 07156) and B27 (Life Technologies, 17504044) containing CHIR (3 mM, Tocris Bioscience, 4423) and FGF2 (10 ng/ml, R&D Systems, 233-FB-001MG/CF) and NRG1 (10 ng/ml, R&D 378-SM-025). After 14 days of suspension culture, the spheroids were plated on Poly Ornithine/Laminin/Fibronectin (PO/LM/FN) coated dishes (prepared as described previously (17)) in Neurobasal (NB) medium supplemented with L-Glutamine (Gibco, 25030-164), N2 (Stem Cell Technologies, 07156) and B27 (Life Technologies, 17504044) containing NRG1 (20 ng/ml, R&D 378-SM-025), FGF2 (10 ng/ml, R&D Systems, 233-FB-001MG/CF) and cAMP (100 mM, Sigma, D0260). The SC precursors migrate out of the plated spheroids and differentiate into SCs within 10 days. For long-term expansion, cells were cultured in Schwann cell medium (Sciencell, 1701 without FBS) on PO/LM/FN coated dishes. Cells were fixed for immunostaining or harvested for gene expression analysis at Day 25, Day 35, Day 50 and Day 100 of differentiation.

### NC Induction Option 2

Differentiations of hPSCs towards NC were carried out following previously established methods using the chemically defined Essential 6 (E6, Life Technologies, A1516401) medium.13 Briefly, when the monolayer culture of hPSCs reached about 70% confluency, NC induction protocol was initiated (D0) by aspirating the maintenance medium (E8, Life Technologies, A2858501) and replacing it with NC induction medium A [BMP4 (1 ng ml-1, R&D Systems, 314-BP), SB431542 (10 mM, R&D Systems, 1614), and CHIR 99021 (600 nM, Tocris Bioscience, 4423) in Essential 6 medium (Life Technologies, A1516401)]. Subsequently, on D2-D12 cultures were fed with NC induction medium B [SB431542 (10 mM) and CHIR 99021 (1.5 mM) in Essential 6 medium].

### Induction and Expansion of Schwann Cells Option 2

On D12, NC are dissociated to form 3D spheroids and maintained until D30 to facilitate the emergence of glial progenitors. To do so, we removed medium B on D12 and detached the NC monolayers using Accutase (Innovative Cell Technologies, AT104) for 30 min at 37° C., 5% CO2. After centrifuging the samples at 290×g for 1 min, we re-suspended the cells in NC-C medium [FGF2 (10 ng ml-1, R&D

Systems, 233-FB-001MG/CF), CHIR 99021 (3 mM), N2 supplement (10 ml ml-1, CTS, A1370701), B27 supplement (20 ml ml-1, Life

Technologies, 17504044), glutagro (10 ml ml-1, Corning, 25-015-CI), and MEM NEAAs (10 ml ml-1, Corning, 25-025-CI) in neurobasal medium (Life Technologies, 21103049)] and transferred them to ultra-low-attachment plates (Fisher Scientific, 3471) to form free-floating 3D developing precursors. Two days later, when the free-floating developing precursors could be observed, we gently gathered them in the center of each well using a swirling motion. Then, the old media was carefully aspirated from the circumference of each well without removing developing precursors. After addition of the fresh NC-C medium, the cultures were incubated for 48 h (37° C. and 5% CO2) prior to passaging using Accutase. Similarly, cultures were fed with fresh medium every other day and passaged every four days until D30. On D30, the free-floating developing precursors were dissociated using Accutase and cultured in Schwann cell medium (Sciencell, 1701) without FBS, plated on dishes coated with Poly Ornithine/Laminin/Fibronectin (PO/LM/FN, Sigma, P3655, Cultrex 3400-10 and Corning, 356008 at 15 mg/ml, 2 mg/ml, 2 mg/ml respectively). Typically, starting with one 6-well plate on D0 should yield three 10-cm PO/LM/FN coated dishes on D30. Cells were treated with Schwann Cell medium without FBS with 10 mM SB431542 at D32-D42 and 100 mM cAMP (Sigma, D0260) at D32-D39 to improve induction efficiency and purity. Cultures were fed with fresh medium every day and passaging was done using 0.05% trypsin (Fisher Scientific, MT25052CI) when the cultures expanded and became confluent (>80%). Long-term maintenance and expansion of cells were in Schwann cell medium without FBS.

### FACS and Immunofluorescence (IF) Analysis

For IF, the cells were fixed with 4% paraformaldehyde (PFA, SCBT, sc-281692) for 20 minutes, then blocked and permeabilized using 1% Bovine Serum Albumin (BSA, Thermo Scientific, 23209) and 0.3% triton X-100 (Sigma, T8787). The cells were then incubated in primary antibody solutions overnight at 4° C. and stained with fluorophore conjugated secondary antibodies at RT for 1 hour, the stained cells were then incubated with DAPI (1 ng/ml, Sigma, D9542-5MG) and washed several times before imaging. For Flow Cytometry analysis, the cells were dissociated with Accutase (Innovative Cell Technologies, AT104) and fixed and permeabilized using BD Cytofix/Cytoperm (BD Bioscience, 554722) solution, then washed, blocked and permeabilized using BD Perm/Wash buffer (BD Bioscience, 554723) according to the manufacturer's instructions. The cells were then stained with primary (overnight at 4° C.) and secondary (30 min at room temperature) antibodies and analyzed using a flow cytometer (FlowJo software). The antibodies and the dilutions used are as follows: CD49D (Biolegend, 304301, 1:800), CHAT (Proteintech, 20747-1-AP, 1:1000), CHAT (Sigma, AB144P, 1:1000), GFAP (Abcam, ab4674, 1:1000), MAG (Millipore, LS-C279052-200, 1:200), MBP (Millipore, MAB386, 1:200), MPZ (abeam, ab39375, 1:500), NFH (Encor, RPCA-NF-H, 1:1000), NFkB p65 (Invitrogen, 710048, 1:500), PMP22 (Novus Biologicals, NB110-59086, 1:100), S100 (Thermo Scientific, RB-9018-P0, 1:500), SC101 (Takara, Y40400, 1:1000), TUBB3 (Millipore Sigma, ab9354, 1:350), TUBB3 (Biolegend, 801202, 1:1500).

1. A composition comprising one or a plurality of Schwann cells, wherein the Schwann cells comprise:
(i) CD98 or a functional fragment thereof that comprises at least about 70% sequence identity to CD98;
(ii) MPZ, MAG, PMPP22, PLLP or a functional fragment thereof that comprises at least about 70% sequence identity to MPZ, MAG, PMPP22, PLLP; or
(iii) POU6F2, CD44, CD81 or a functional fragment thereof that comprises at least about 70% sequence identity to POU6F2, CD44, and CD81.

2. (canceled)

3. The composition of claim 1, wherein the cell is in culture no fewer than about 35 days.

4. (canceled)

5. The composition of claim 1, wherein the composition further comprises one or a combination of S100, myelin binding protein (MBP), and GFAP.

6. The composition of claim 1, wherein the cell further comprises one or a combination of: SOX10, POU3F2, GAP43, or a functional fragment thereof that comprises at least about 70% sequence identity to SOX10, POU3F2, and GAP43.

7. The composition of claim 1, wherein the cell further comprises one or a combination of PMP22, SOX10, POU3F2, GAP43, NGFR, MP2, CD46, CD146, CD147, CD166, ERBB3, GDNF, or a functional fragment thereof that comprises at least about 70% sequence identity to PMP22, SOX10, POU3F2, GAP43, NGFR, MP2, CD46, CD146, CD147, CD166, ERBB3, and GDNF.

8. The composition of claim 7, wherein the cell further comprises one or a combination of FOX01, TBX19, MATN2, PLAT, or a functional fragment thereof that comprises at least about 70% sequence identity to FOX01, TBX19, MATN2, and PLAT.

9. The composition of claim 1, wherein the cell further comprises one or a combination of PMP22, POU3F2, GAP43, NGFR, MP2, CD46, CD146, CD147, CD166, ERBB3, GDNF, CD9, CD49e, CD171, or a functional fragment thereof that comprises at least about 70% sequence identity to one of PMP22, POU3F2, GAP43, NGFR, MP2, CD46, CD146, CD147, CD166, ERBB3, GDNF, CD9, CD49e, and CD171.

10.-11. (canceled)

12. The composition of claim 1, wherein the composition comprises greater than about 70% Schwann cells.

13-15. (canceled)

16. The composition of claim 1, wherein the cells are in culture at least about 2 weeks.

17. A pharmaceutical composition comprising:
(i) a composition of claim 1 comprising a therapeutically effective amount of Schwann cells; and
(ii) a pharmaceutically acceptable carrier.

18.-20. (canceled)

21. A method of differentiating or enriching Schwann cells in a cell culture comprising exposing a composition of pluripotent stem cells with FGF2 for a time period sufficient for the neural crest cell to express SOX10 or a functional fragment thereof.

22. The method of claim 21 further comprising exposing the composition of neural crest cells with a WNT pathway activator for a time period sufficient for the neural crest cell to express SOX10 or a functional fragment thereof.

23. The method claim 21 further comprising exposing the composition of neural crest cells with SB431542 and/or dbcAMP for a time period sufficient for the neural crest cell to express one or combination of POU3F1, PMP22, MBP, MPZ, AQP4, or a functional fragment thereof.

24. The method of claim 23, wherein the step of exposing the composition of neural crest cells with SB431542 and/or dbcAMP comprises exposure for a time period sufficient for the neural crest cell to differentiate into a Schwann cell.

25. The method of claim 21, wherein the one or plurality of steps of exposing are performed cumulatively for more than about 19 days.

26.-28. (canceled)

29. A method for screening one or more agents for neuromodulatory activity comprising
i) culturing the composition of claim 1 in a tissue culture system comprising one or a plurality of healthy or dysfunctional neural cells;
ii) exposing the composition to one or more agents;
iii) monitoring the composition for neuromodulating activity; and
iv) identifying the one more agents as toxic to cells of the nervous system if the neuromodulatory activity inhibits or disrupts or reduces growth or health of healthy neural cells as compared to the neuromodulatory activity of the cells in the absence of the one or more agents; or identifying the one more agents as inducing repair of neural cells if the neuromodulatory activity improves or restores function of dysfunctional neural cells as compared to the neuromodulatory activity of the dysfunctional cells in the absence of the one or more agents.

30. (canceled)

31. A method of treating a spinal cord injury or diabetic peripheral neuropathy in a subject in need thereof comprising administering to the subject a pharmaceutical composition comprising: (i) a therapeutically effective amount of Schwann cells and a pharmaceutically effective carrier; or (ii) a therapeutically effective amount of bupropion or a derivative or salt thereof; and a pharmaceutically effective carrier.

32. The method of claim 31, wherein the composition is administered intravenously.

33.-41. (canceled)


SEQUENCE LISTING (61 sequences)

SEQ ID NO: 1 - protein, 507 aa, Homo sapiens
MAGAGPKRRALAAPAAEEKEEAREKMLAAKSADGSAPAGEGEGVTLQRNITLLNGVAIIVGTIIGSGIFVTPTGVLKEAGSPGLALVVWAACGVFSIVGALCYAELGTTISKSGGDYAYMLEVYGSLPAFLKLWIELLIIRPSSQYIVALVFATYLLKPLFPTCPVPEEAAKLVACLCVLLLTAVNCYSVKAATRVQDAFAAAKLLALALIILLGFVQIGKGDVSNLDPNFSFEGTKLDVGNIVLALYSGLFAYGGWNYLNFVTEEMINPYRNLPLAIIISLPIVTLVYVLTNLAYFTTLSTEQMLSSEAVAVDFGNYHLGVMSWIIPVFVGLSCFGSVNGSLFTSSRLFFVGSREGHLPSILSMIHPQLLTPVPSLVFTCVMTLLYAFSKDIFSVINFFSFFNWLCVALAIIGMIWLRHRKPELERPIKVNLALPVFFILACLFLIAVSFWKTPVECGIGFTIILSGLPVYFFGVWWKNKPKWLLQGIFSTTVLCQKLMQVVPQET

SEQ ID NO: 2 - protein, 94 aa, Homo sapiens
MGSELETAMETLINVFHAHSGKEGDKYKLSKKELKELLQTELSGFLDAQKDVDAVDKVMKELDENGDGEVDFQEYVVLVAALTVACNNFFWENS

SEQ ID NO: 3 - protein, 197 aa, Homo sapiens
MASQKRPSQRHGSKYLATASTMDHARHGFLPRHRDTGILDSIGRFFGGDRGAPKRGSGKVPWLKPGRSPLPSHARSQPGLCNMYKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 4 - protein, 472 aa, Homo sapiens
MERRRITSAARRSYVSSGEMMVGGLAPGRRLGPGTRLSLARMPPPLPTRVDFSLAGALNAGFKETRASERAEMMELNDRFASYIEKVRFLEQQNKALAAELNQLRAKEPTKLADVYQAELRELRLRLDQLTANSARLEVERDNLAQDLATVRQKLQDETNLRLEAENNLAAYRQEADEATLARLDLERKIESLEEEIRFLRKIHEEEVRELQEQLARQQVHVELDVAKPDLTAALKEIRTQYEAMASSNMHEAEEWYRSKFADLTDAAARNAELLRQAKHEANDYRRQLQSLTCDLESLRGTNESLERQMREQEERHVREAASYQEALARLEEEGQSLKDEMARHLQEYQDLLNVKLALDIEIATYRKLLEGEENRITIPVQTFSNLQIRGGKSTKDGENHKVTRYLKSLTIRVIPIQAHQIVNGTPPARETSLDTKSVSEGHLKRNIVVKTVEMRDGEVIKESKQEHKDVM

SEQ ID NO: 5 - protein, 160 aa, Homo sapiens
MLLLLLSIIVLHVAVLVLLFVSTIVSQWIVGNGHATDLWQNCSTSSSGNVHHCFSSSPNEWLQSVQATMILSIIFSILSLFLFFCQLFTLTKGGRFYITGIFQILAGLCVMSAAAIYTVRHPEWHLNSDYSYGFAYILAWVAFPLALLSGVIYVILRKRE

SEQ ID NO: 6 - protein, 668 aa, Homo sapiens
MWLFFGITGLLTAALSGHPSPAPPDQLNTSSAESELWEPGERLPVRLTNGSSSCSGTVEVRLEASWEPACGALWDSRAAEAVCRALGCGGAEAASQLAPPTPELPPPPAAGNTSVAANATLAGAPALLCSGAEWRLCEVVEHACRSDGRRARVTCAENRALRLVDGGGACAGRVEMLEHGEWGSVCDDTWDLEDAHVVCRQLGCGWAVQALPGLHFTPGRGPIHRDQVNCSGAEAYLWDCPGLPGQHYCGHKEDAGAVCSEHQSWRLTGGADRCEGQVEVHFRGVWNTVCDSEWYPSEAKVLCQSLGCGTAVERPKGLPHSLSGRMYYSCNGEELTLSNCSWRFNNSNLCSQSLAARVLCSASRSLHNLSTPEVPASVQTVTIESSVTVKIENKESRELMLLIPSIVLGILLLGSLIFIAFILLRIKGKYALPVMVNHQHLPTTIPAGSNSYQPVPITIPKEVFMLPIQVQAPPPEDSDSGSDSDYEHYDFSAQPPVALTTFYNSQRHRVTDEEVQQSRFQMPPLEEGLEELHASHIPTANPGHCITDPPSLGPQYHPRSNSESSTSSGEDYCNSPKSKLPPWNPQVFSSERSSFLEQPPNLELAGTQPAFSAGPPADDSSSTSSGEWYQNFQPPPQPPSEEQFGCPGSPSPQPDSTDNDDYDDISAA

SEQ ID NO: 7 - protein, 249 aa, Homo sapiens
MPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYAVCRLGKDTLLRFLRIVSAHRVLTAPDQCKHSNRLLTICFSHPHPCLLAVVELLWFGWGRGTVYLRHLPQEGRTRNLHREGKLRPGSWCPCPHCSGQTLQA

SEQ ID NO: 8 - protein, 700 aa, Homo sapiens
MDKFWWHAAWGLCLVPLSLAQIDLNITCRFAGVFHVEKNGRYSISRTEAADLCKAFNSTLPTMAQMEKALSIGFETCRYGFIEGHVVIPRIHPNSICAANNTGVYILTSNTSQYDTYCFNASAPPEEDCTSVTDLPNAFDGPITITIVNRDGTRYVQKGEYRTNPEDIYPSNPTDDDVSSGSSSERSSTSGGYIFYTFSTVHPIPDEDSPWITDSTDRIPATTLMSTSATATETATKRQETWDWFSWLFLPSESKNHLHTTTQMAGTSSNTISAGWEPNEENEDERDRHLSFSGSGIDDDEDFISSTISTTPRAFDHTKQNQDWTQWNPSHSNPEVLLQTTTRMTDVDRNGTTAYEGNWNPEAHPPLIHHEHHEEEETPHSTSTIQATPSSTTEETATQKEQWFGNRWHEGYRQTPKEDSHSTTGTAAASAHTSHPMQGRTTPSPEDSSWTDFFNPISHPMGRGHQAGRRMDMDSSHSITLQPTANPNTGLVEDLDRTGPLSMTTQQSNSQSFSTSHEGLEEDKDHPTTSTLTSSNRNDVTGGRRDPNHSEGSTTLLEGYTSHYPHTKESRTFIPVTSAKTGSFGVTAVTVGDSNSNVNRSLSGDQDTFHPSGGSHTTHGSESDGHSHGSQEGGANTTSGPIRTPQIPEWLIILASLLALALILAVCIAVNSRRRCGQKKKLVINSGNGAVEDRKPSGLN

SEQ ID NO: 9 - protein, 399 aa, Homo sapiens
MEPPGRRECPFPSWRFPGLLLAAMVLLLYSFSDACEEPPTFEAMELIGKPKPYYEIGERVDYKCKKGYFYIPPLATHTICDRNHTWLPVSDDACYRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKVLPPSSTKPPALSHSVSTSSTTKSPASSASGPRPTYKPPVSNYPGYPKPEEGILDSLDVWVIAVIVIAIVVGVAVICVVPYRYLQRRKKKGKADGGAEYATYQTKSTTPAEQRG

SEQ ID NO: 10 - protein, 1049 aa, Homo sapiens
MGSRTPESPLHAVQLRWGPRRRPPLLPLLLLLLPPPPRVGGFNLDAEAPAVLSGPPGSFFGFSVEFYRPGTDGVSVLVGAPKANTSQPGVLQGGAVYLCPWGASPTQCTPIEFDSKGSRLLESSLSSSEGEEPVEYKSLQWFGATVRAHGSSILACAPLYSWRTEKEPLSDPVGTCYLSTDNFTRILEYAPCRSDFSWAAGQGYCQGGFSAEFTKTGRVVLGGPGSYFWQGQILSATQEQIAESYYPEYLINLVQGQLQTRQASSIYDDSYLGYSVAVGEFSGDDTEDFVAGVPKGNLTYGYVTILNGSDIRSLYNFSGEQMASYFGYAVAATDVNGDGLDDLLVGAPLLMDRTPDGRPQEVGRVYVYLQHPAGIEPTPTLTLTGHDEFGRFGSSLTPLGDLDQDGYNDVAIGAPFGGETQQGVVFVFPGGPGGLGSKPSQVLQPLWAASHTPDFFGSALRGGRDLDGNGYPDLIVGSFGVDKAVVYRGRPIVSASASLTIFPAMFNPEERSCSLEGNPVACINLSFCLNASGKHVADSIGFTVELQLDWQKQKGGVRRALFLASRQATLTQTLLIQNGAREDCREMKIYLRNESEFRDKLSPIHIALNFSLDPQAPVDSHGLRPALHYQSKSRIEDKAQILLDCGEDNICVPDLQLEVFGEQNHVYLGDKNALNLTFHAQNVGEGGAYEAELRVTAPPEAEYSGLVRHPGNFSSLSCDYFAVNQSRLLVCDLGNPMKAGASLWGGLRFTVPHLRDTKKTIQFDFQILSKNLNNSQSDVVSFRLSVEAQAQVTLNGVSKPEAVLFPVSDWHPRDQPQKEEDLGPAVHHVYELINQGPSSISQGVLELSCPQALEGQQLLYVTRVTGLNCTTNHPINPKGLELDPEGSLHHQQKREAPSRSSASSGPQILKCPEAECFRLRCELGPLHQQESQSLQLHFRVWAKTFLQREHQPFSLQCEAVYKALKMPYRILPRQLPQKERQVATAVQWTKAEGSYGVPLWIIILAILFGLLLLGLLIYILYKLGFFKRSLPYGTAMEKAQLKPPATSDA

SEQ ID NO: 11 - protein, 236 aa, Homo sapiens
MGVEGCTKCIKYLLFVFNFVFWLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVY

SEQ ID NO: 12 - protein, 646 aa, Homo sapiens
MGLPRLVCAFLLAACCCCPRVAGVPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQLRVYKAPEEPNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQGLDLDTMISLLSEPQELLVNYVSDVRVSPAAPERQEGSSLTLTCEAESSQDLEFQWLREETGQVLERGPVLQLHDLKREAGGGYRCVASVPSIPGLNRTQLVNVAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTERKLPEPESRGVVIVAVIVCILVLAVLGAVLYFLYKKGKLPCRRSGKQEITLPPSRKSELVVEVKSDKLPEEMGLLQGSSGDKRAPGDQGEKYIDLRH

SEQ ID NO: 13 - protein, 385 aa, Homo sapiens
MAAALFVLLGFALLGTHGASGAAGFVQAPLSQQRWVGGSVELHCEAVGSPVPEIQWWFEGQGPNDTCSQLWDGARLDRVHIHATYHQHAASTISIDTLVEEDTGTYECRASNDPDRNHLTRAPRVKWVRAQAVVLVLEPGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 14 - protein, 583 aa, Homo sapiens
MESKGASSCRLLFCLLISATVFRPGLGWYTVNSAYGDTIIIPCRLDVPQNLMFGKWKYEKPDGSPVFIAFRSSTKKSVQYDDVPEYKDRLNLSENYTLSISNARISDEKRFVCMLVTEDNVFEAPTIVKVFKQPSKPEIVSKALFLETEQLKKLGDCISEDSYPDGNITWYRNGKVLHPLEGAVVIIFKKEMDPVTQLYTMTSTLEYKTTKADIQMPFTCSVTYYGPSGQKTIHSEQAVFDIYYPTEQVTIQVLPPKNAIKEGDNITLKCLGNGNPPPEEFLFYLPGQPEGIRSSNTYTLTDVRRNATGDYKCSLIDKKSMIASTAITVHYLDLSLNPSGEVTRQIGDALPVSCTISASRNATVVWMKDNIRLRSSPSFSSLHYQDAGNYVCETALQEVEGLKKRESLTLIVEGKPQIKMTKKTDPSGLSKTIICHVEGFPKPAIQWTITGSGSVINQTEESPYINGRYYSKIIISPEENVTLTCTAENQLERTVNSLNVSAISIPEHDEADEISDENREKVNDQAKLIVGIVVGLLLAALVAGVVYWLYMKKSKTASKHVNKDLGNMEENKKLEENNHKTEA

SEQ ID NO: 15 - protein, 1257 aa, Homo sapiens
MVVALRYVWPLLLCSPCLLIQIPEEYEGHHVMEPPVITEQSPRRLVVFPTDDISLKCEASGKPEVQFRWTRDGVHFKPKEELGVTVYQSPHSGSFTITGNNSNFAQRFQGIYRCFASNKLGTAMSHEIRLMAEGAPKWPKETVKPVEVEEGESVVLPCNPPPSAEPLRIYWMNSKILHIKQDERVTMGQNGNLYFANVLTSDNHSDYICHAHFPGTRTIIQKEPIDLRVKATNSMIDRKPRLLFPTNSSSHLVALQGQPLVLECIAEGFPTPTIKWLRPSGPMPADRVTYQNHNKTLQLLKVGEEDDGEYRCLAENSLGSARHAYYVTVEAAPYWLHKPQSHLYGPGETARLDCQVQGRPQPEVTWRINGIPVEELAKDQKYRIQRGALILSNVQPSDTMVTQCEARNRHGLLLANAYIYVVQLPAKILTADNQTYMAVQGSTAYLLCKAFGAPVPSVQWLDEDGTTVLQDERFFPYANGTLGIRDLQANDTGRYFCLAANDQNNVTIMANLKVKDATQITQGPRSTIEKKGSRVTFTCQASFDPSLQPSITWRGDGRDLQELGDSDKYFIEDGRLVIHSLDYSDQGNYSCVASTELDVVESRAQLLVVGSPGPVPRLVLSDLHLLTQSQVRVSWSPAEDHNAPIEKYDIEFEDKEMAPEKWYSLGKVPGNQTSTTLKLSPYVHYTFRVTAINKYGPGEPSPVSETVVTPEAAPEKNPVDVKGEGNETTNMVITWKPLRWMDWNAPQVQYRVQWRPQGTRGPWQEQIVSDPFLVVSNTSTFVPYEIKVQAVNSQGKGPEPQVTIGYSGEDYPQAIPELEGIEILNSSAVLVKWRPVDLAQVKGHLRGYNVTYWREGSQRKHSKRHIHKDHVVVPANTTSVILSGLRPYSSYHLEVQAFNGRGSGPASEFTFSTPEGVPGHPEALHLECQSNTSLLLRWQPPLSHNGVLTGYVLSYHPLDEGGKGQLSFNLRDPELRTHNLTDLSPHLRYRFQLQATTKEGPGEAIVREGGTMALSGISDFGNISATAGENYSVVSWVPKEGQCNFRFHILFKALGEEKGGASLSPQYVSYNQSSYTQWDLQPDTDYEIHLFKERMFRHQMAVKTNGTGRVRLPPAGFATEGWFIGFVSAIILLLLVLLILCFIKRSKGGKYSVKDKEDTQVDSEARPMKDETFGEYRSLESDNEEKAFGSSQPSLNGDIKPLGSDDSLADYGGSVDVQFNEDGSFIGQYSGKKEKEAAGGNDSSGATSPINPAVALE

SEQ ID NO: 16 - protein, 427 aa, Homo sapiens
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNSCKQNKQGANSRPVNQTPPPEGEKLHSDSGISVDSQSLHDQQPHTQTASGQALKGDGGLYSSLPPAKREEVEKLLNGSAGDTWRHLAGELGYQPEHIDSFTHEACPVRALLASWATQDSATLDALLAALRRIQRADLVESLCSESTATSPV

SEQ ID NO: 17 - protein, 466 aa, Homo sapiens
MAEEQDLSEVELSPVGSEEPRCLSPGSAPSLGPDGGGGGSGLRASPGPGELGKVKKEQQDGEADDDKFPVCIREAVSQVLSGYDWTLVPMPVRVNGASKSKPHVKRPMNAFMVWAQAARRKLADQYPHLHNAELSKTLGKLWRLLNESDKRPFIEEAERLRMQHKKDHPDYKYQPRRRKNGKAAQGEAECPGGEAEQGGTAAIQAHYKSAHLDHRHPGEGSPMSDGNPEHPSGQSHGPPTPPTTPKTELQSGKADPKRDGRSMGEGGKPHIDFGNVDIGEISHEVMSNMETFDVAELDQYLPPNGHPGHVSSYSAAGYGLGSALAVASGHSAWISKPPGVALPTVSPPGVDAKAQVKTETAGPQGPPHYTDQPSTSQIAYTSLSLPHYGSAFPSISRPQFDYSDHQPSGPYYGHSGQASGLYSAFSYMGPSQRPLYTAISDPSPSGPQSHSPTHWEQPVYTTLSRP

SEQ ID NO: 18 - protein, 443 aa, Homo sapiens
MATAASNHYSLLTSSASIVHAEPPGGMQQGAGGYREAQSLVQGDYGALQSNGHPLSHAHQWITALSHGGGGGGGGGGGGGGGGGGGGGDGSPWSTSPLGQPDIKPSVVVQQGGRGDELHGPGALQQQHQQQQQQQQQQQQQQQQQQQQQRPPHLVHHAANHHPGPGAWRSAAAAAHLPPSMGASNGGLLYSQPSFTVNGMLGAGGQPAGLHHHGLRDAHDEPHHADHHPHPHSHPHQQPPPPPPPQGPPGHPGAHHDPHSDEDTPTSDDLEQFAKQFKQRRIKLGFTQADVGLALGTLYGNVFSQTTICRFEALQLSFKNMCKLKPLLNKWLEEADSSSGSPTSIDKIAAQGRKRKKRTSIEVSVKGALESHFLKCPKPSAQEITSLADSLQLEKEVVRVWFCNRRQKEKRMTPPGGTLPGAEDVYGGSRDTPPHHGVQTPVQ

SEQ ID NO: 19 - protein, 312 aa, Homo sapiens
MAPGAPSSSPSPILAVLLFSSLVLSPAQAIVVYTDREVHGAVGSRVTLHCSFWSSEWVSDDISFTWRYQPEGGRDAISIFHYAKGQPYIDEVGTFKERIQWVGDPRWKDGSIVIHNLDYSDNGTFTCDVKNPPDIVGKTSQVTLYVFEKVPTRYGVVLGAVIGGVLGVVLLLLLLFYVVRYCWLRRQAALQRRLSAMEKGKLHKPGKDASKRGRQTPVLYAMLDHSRSTKAVSEKKAKGLGESRKDKKXRLAGRAGDRGLGVESAKGPKVMVIEMELRKDEQSPELRPAVKSPSRTSLKNALKNMMGLNSDK

SEQ ID NO: 20 - protein, 274 aa, Homo sapiens
MTKSCSELCHPALHFLPCLGGLRKNLQRAVRPSPYSLGFLTFWISRVEKNDDDQKIEQDGIKPEDKAHKAATKIQASFRGHITRKKLKGEKKDDVQAAEAEANKKDEAPVADGVEKKGEGTTTAEAAPATGSKPDEPGKAGETPSEEKKGEGDAATEQAAPQAPASSEEKAGSAETESATKASTDNSPSSKAEDAPAKEEPKQADVPAAVTAAAATTPAAEDAAAKATAQPPTETGESSQAEENIEAVDETKPKESARQDEGKEEEPEADQEHA

SEQ ID NO: 21 - protein, 1342 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 22 - protein, 228 aa, Homo sapiens
MQSLPNSNGAAAGRDFKMKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 23 - protein, 626 aa, Homo sapiens
MIFLTALPLFWIMISASRGGHWGAWMPSSISAFEGTCVSIPCRFDFPDELRPAVVHGVWYFNSPYPKNYPPVVFKSRTQVVHESFQGRSRLLGDLGLRNCTLLLSNVSPELGGKYYFRGDLGGYNQYTFSEHSVLDIVNTPNIVVPPEVVAGTEVEVSCMVPDNCPELRPELSWLGHEGLGEPAVLGRLREDEGTWVQVSLLHFVPTREANGHRLGCQASFPNTTLQFEGYASMDVKYPPVIVEMNSSVEAIEGSHVSLLCGADSNPPPLLTWMRDGTVLREAVAESLLLELEEVTPAEDGVYACLAENAYGQDNRTVGLSVMYAPWKPTVNGTMVAVEGETVSILCSTQSNPDPILTIFKEKQILSTVIYESELQLELPAVSPEDDGEYWCVAENQYGQRATAFNLSVEFAPVLLLESHCAAARDTVQCLCVVKSNPEPSVAFELPSRNVTVNESEREFVYSERSGLVLTSILTLRGQAQAPPRVICTARNLYGAKSLELPFQGAHRLMWAKIGPVGAVVAFAILIAIVCYITQTRRKKNVTESPSFSAGDNPPVLFSSDFRISGAPEKYESERRLGSERRLLGLRGEPPELDLSYSHSDLGKRPTKDSYTLTEELAEYAEIRVK

SEQ ID NO: 24 - protein, 182 aa, Homo sapiens
MAEFPSKVSTRTSSPAQGAEASVSALRPDLGFVRSRLGALMLLQLVLGLLVWALIADTPYHLYPAYGWVMFVAVFLWLVTIVLFNLYLFQLHMKLYMVPWPLVLMIFNISATVLYITAFIACSAAVDLTSLRGTRPYNQRAAASFFACLVMIAYGVSAFFSYQAWRGVGSNAATSQMAGGYA

SEQ ID NO: 25 - protein, 720 aa, Homo sapiens
MHARNPHSPFQRQHMMDCYLSAQQDTGTMQAVIGQDPMIAGQVSKPLLSVRSEMNAELRGEDKAATSDSELNEPLLAPVESNDSEDTPSKLFGARGNPALSDPGTPDQHQASQTHPPFPVGPQPLLTAQQLASAVAGVMPGGPPALNQPILIPFNMAGQLGGQQGLVLTLPTANLTNIQGLVAAAAAGGIMTLPLQNLQATSSLNSQLQQLQLQLQQQQQQQQQQPPPSTNQHPQPAPQAPSQSQQQPLQPTPPQQPPPASQQPPAPTSQLQQAPQPQQHQPHSHSQNQNQPSPTQQSSSPPQKPSQSPGHGLPSPLTPPNPLQLVNNPLASQAAAAAAAMSSIASSQAFGNALSSLQGVTGQLVTNAQGQIIGTIPLMPNPGPSSQAASGTQGLQVQPITPQLLTNAQGQIIATVIGNQILPVINTQGITLSPIKPGQQLHQPSQTSVGQAASQGNLLHLAHSQASMSQSPVRQASSSSSSSSSSSALSVGQLVSNPQTAAGEVDGVNLEEIREFAKAFKIRRLSLGLTQTQVGQALSATEGPAYSQSAICRHTILRSHFFLPQEAQENTIASSLTAKLNPGLLYPARFEKLDITPKSAQKIKPVLERWMAEAEARHRAGMQNLTEFIGSEPSKKRKRRTSFTPQALEILNAHFEKNTHPSGQEMTEIAEKLNYDREVVRVWFCNKRQALKNTIKRLKQHEPATAVPLEPLTDSLEENS

SEQ ID NO: 26 - protein, 1932 aa, Homo sapiens
MELTPASSLTCSLLSPRLPGSFPQLRRVPPCSRPWLPKAPVMARWPPFGLCLLLLLLSPPPLPLTGAHRFSAPNTTLNHLALAPGRGTLYVGAVNRLFQLSPELQLEAVAVTGPVIDSPDCVPFRDPAECPQAQLTDNANQLLLVSSRAQELVACGQVRQGVCETRRLGDVAEVLYQAEDPGDGQFVAANTPGVATVGLVVPLPGRDLLLVARGLAGKLSAGVPPLAIRQLAGSQPFSSEGLGRLVVGDFSDYNNSYVGAFADARSAYFVFRRRGARAQAEYRSYVARVCLGDTNLYSYVEVPLACQGQGLIQAAFLAPGTLLGVFAAGPRGTQAALCAFPMVELGASMEQARRLCYTAGGRGPSGAEEATVEYGVTSRCVTLPLDSPESYPCGDEHTPSPIAGRQPLEVQPLLKLGQPVSAVAALQADGHMIAFLGDTQGQLYKVFLHGSQGQVYHSQQVGPPGSAISPDLLLDSSGSHLYVLTAHQVDRIPVAACPQFPDCASCLQAQDPLCGWCVLQGRCTRKGQCGRAGQLNQWLWSYEEDSHCLHIQSLLPGHHPRQEQGQVTLSVPRLPILDADEYFHCAFGDYDSLAHVEGPHVACVTPPQDQVPLNPPGTDHVTVPLALMFEDVTVAATNFSFYDCSAVQALEAAAPCRACVGSIWRCHWCPQSSHCVYGEHCPEGERTIYSAQEVDIQVRGPGACPQVEGLAGPHLVPVGWESHLALRVRNLQHFRGLPASFHCWLELPGELRGLPATLEETAGDSGLIHCQAHQFYPSMSQRELPVPIYVTQGEAQRLDNTHALYVILYDCAMGHPDCSHCQAANRSLGCLWCADGQPACRYGPLCPPGAVELLCPAPSIDAVEPLTGPPEGGLALTILGSNLGRAFADVQYAVSVASRPCNPEPSLYRTSARIVCVTSPAPNGTTGPVRVAIKSQPPGISSQHFTYQDPVLLSLSPRWGPQAGGTQLTIRGQHLQTGGNTSAFVGGQPCPILEPVCPEAIVCRTRPQAAPGEAAVLVVFGHAQRTLLASPFRYTANPQLVAAEPSASFRGGGRLIRVRGTGLDVVQRPLLSVWLEADAEVQASRAQPQDPQPRRSCGAPAADPQACIQLGGGLLQCSTVCSVNSSSLLLCRSPAVPDRAHPQRVFFTLDNVQVDFASASGGQGFLYQPNPRLAPLSREGPARPYRLKPGHVLDVEGEGLNLGISKEEVRVHIGRGECLVKTLTRTHLYCEPPAHAPQPANGSGLPQFVVQMGNVQLALGPVQYEAEPPLSAFPVEAQAGVGMGAAVLIAAVLLLTLMYRHKSKQALRDYQKVLVQLESLETGVGDQCRKEFTDLMTEMTDLSSDLEGSGIPFLDYRTYAERAFFPGHGGCPLQPKPEGPGEDGHCATVRQGLTQLSNLLNSKLFLLTLIHTLEEQPSFSQRDRCHVASLLSLALHGKLEYLTDIMRTLLGDLAAHYVHRNPKLMLRRTETMVEKLLTNWLSICLYAFLREVAGEPLYMLFRAIQYQVDKGPVDAVTGKAKRTLNDSRLLREDVEFQPLTLMVLVGPGAGGAAGSSEMQRVPARVLDTDTITQVKEKVLDQVYKGTPFSQRPSVHALDLEWRSGLAGHLTLSDEDLTSVTQNHWKRLNTLQHYKVPDGATVGLVPQLHRGSTISQSLAQRCPLGENIPTLEDGEEGGVCLWHLVKATEEPEGAKVRCSSLREREPARAKAIPEIYLTRLLSMKGTLQKFVDDTFQAILSVNRPIPIAVKYLFDLLDELAEKHGIEDPGTLHIWKTNSLLLRFWVNALKNPQLIFDVRVSDNVDAILAVIAQTFIDSCTTSEHKVGRDSPVNKLLYAREIPRYKQMVERYYADIRQSSPASYQEMNSALAELSGNYTSAPHCLEALQELYNHIHRYYDQIISALEEDPVGQKLQLACRLQQVAALVENKVTDL

SEQ ID NO: 27 - RNA, 2224 nt, Homo sapiens
  source 1..2224: mol_type = mRNA
gcattgcggcttggttttctcacccagtgcatgtggcaggagcggtgagatcactgcctcacggcgatcctggactgacggtcacgactgcctaccctctaaccctgttctgagctgccccttgcccacacaccccaaacctgtgtgcaggatccgcctccatggagctacagcctcctgaagcctcgatcgccgtcgtgtcgattccgcgccagttgcctggctcacattcggaggctggtgtccagggtctcagcgcgggggacgactcagagacggggtctgactgtgttacccaggctggtcttcaactcttggcctcaagtgatcctcctgccttagcttccaagaatgctgaggttacagtagaaacggggtttcaccatgttagccaggctgatattgaattcctgacctcaattgatccgactgcctcggcctccggaagtgctgggattacaggcaccatgagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgagcgccgcgttgtcgcgagctaccggcgcagaagtggtggcacacgggcgccctctaccgcatcggcgaccttcaggccttccagggccacggcgcgggcaacctggcgggtctgaaggggcgtctcgattacctgagctctctgaaggtgaagggccttgtgctgggtccaattcacaagaaccagaaggatgatgtcgctcagactgacttgctgcagatcgaccccaattttggctccaaggaagattttgacagtctcttgcaatcggctaaaaaaaagagcatccgtgtcattctggaccttactcccaactaccggggtgagaactcgtggttctccactcaggttgacactgtggccaccaaggtgaaggatgctctggagttttggctgcaagctggcgtggatgggttccaggttcgggacatagagaatctgaaggatgcatcctcattcttggctgagtggcaaaatatcaccaagggcttcagtgaagacaggctcttgattgcggggactaactcctccgaccttcagcagatcctgagcctactcgaatccaacaaagacttgctgttgactagctcatacctgtctgattctggttctactggggagcatacaaaatccctagtcacacagtatttgaatgccactggcaatcgctggtgcagctggagtttgtctcaggcaaggctcctgacttccttcttgccggctcaacttctccgactctaccagctgatgctcttcaccctgccagggacccctgttttcagctacggggatgagattggcctggatgcagctgcccttcctggacagcctatggaggctccagtcatgctgtgggatgagtccagcttccctgacatcccaggggctgtaagtgccaacatgactgtgaagggccagagtgaagaccctggctccctcctttccttgttccggcggctgagtgaccagcggagtaaggagcgctccctactgcatggggacttccacgcgttctccgctgggcctggactcttctcctatatccgccactgggaccagaatgagcgttttctggtagtgcttaactttggggatgtgggcctctcggctggactgcaggcctccgacctgcctgccagcgccagcctgccagccaaggctgacctcctgctcagcacccagccaggccgtgaggagggctcccctcttgagctggaacgcctgaaactggagcctcacgaagggctgctgctccgcttcccctacgcggcctgacttcagcctgacatggacccactacccttctcctttccttcccaggccctttggcttctgatttttctcttttttaaaaacaaacaaacaaactgttgcagattatgagtgaacccccaaatagggtgttttctgccttcaaataaaagtcacccctgcatggtgaa

SEQ ID NO: 28 - RNA, 559 nt, Homo sapiens
  source 1..559: mol_type = mRNA
agccacatttgcaaccttggccatctgtccagaacctgctcccacctcaggcccaggccaaccgtgcactgctgcaatgggctctgagctggagacggcgatggagaccctcatcaacgtgttccacgcccactcgggcaaagagggggacaagtacaagctgagcaagaaggagctgaaagagctgctgcagacggagctctctggcttcctggatgcccagaaggatgtggatgctgtggacaaggtgatgaaggagctagacgagaatggagacggggaggtggacttccaggagtatgtggtgcttgtggctgctctcacagtggcctgtaacaatttcttctgggagaacagttgagcagacagccacattgggcagcgcccttcctctccaccctcccagacctgcctcttccccctgcttccacctcaccccacttatccctctccataaccccacccttgcccaccccacccccacccccaccaagggcgcaagagtagcggtccaagcctgcaactcatctttcattaaaggcttctctctcaccagcca

SEQ ID NO: 29 - RNA, 2738 nt, Homo sapiens
  source 1..2738: mol_type = mRNA
agtcaccgccgccgcgcgccagagagaagcagcctccggccccggcggcccctgtctcccgaccccggaaggcgaagcaggctgcccggggaccccgcgcgtgggcgcttgaagccgagaccagcctgcccgggcctgggcaggcggagcagggccttggaccccgcggcgcccctcggcctcggagcaacgagcgcagcgccgcctctgaagagccaatccattcaggatgggaaaccacgcaggcaaacgagaattaaatgccgagaaggccagtacgaatagtgaaactaacagaggagaatctgaaaaaaagagaaacctgggtgaactttcacggacaacctcagaggacaacgaagtgttcggagaggcagatgcgaaccagaacaatgggacctcctctcaggacacagcggtgactgactccaagcgcacagcggacccgaagaatgcctggcaggatgcccacccagctgacccagggagccgcccccacttgatccgcctcttttcccgagatgccccggggagggaggacaacaccttcaaagacaggccctctgagtccgacgagctccagaccatccaagaagacagtgcagccacctccgagagcctggatgtgatggcgtcacagaagagaccctcccagaggcacggatccaagtacctggccacagcaagtaccatggaccatgccaggcatggcttcctcccaaggcacagagacacgggcatccttgactccatcgggcgcttctttggcggtgacaggggtgcgcccaagcggggctctggcaaggactcacaccacccggcaagaactgctcactacggctccctgccccagaagtcacacggccggacccaagatgaaaaccccgtagtccacttcttcaagaacattgtgacgcctcgcacaccacccccgtcgcagggaaaggggagaggactgtccctgagcagatttagctggggggccgaaggccagagaccaggatttggctacggaggcagagcgtccgactataaatcggctcacaagggattcaagggagtcgatgcccagggcacgctttccaaaatttttaagctgggaggaagagatagtcgctctggatcacccatggctagacgctgaaaacccacctggttccggaatcctgtcctcagcttcttaatataactgccttaaaactttaatcccacttgcccctgttacctaattagagcagatgacccctcccctaatgcctgcggagttgtgcacgtagtagggtcaggccacggcagcctaccggcaatttccggccaacagttaaatgagaacatgaaaacagaaaacggttaaaactgtccctttctgtgtgaagatcacgttccttcccccgcaatgtgcccccagacgcacgtgggtcttcagggggccaggtgcacagacgtccctccacgttcacccctccacccttggactttcttttcgccgtggctgcggcacccttgcgcttttgctggtcactgccatggaggcacacagctgcagagacagagaggacgtgggcggcagagaggactgttgacatccaagcttcctttgtttttttttcctgtccttctctcacctcctaaagtagacttcatttttcctaacaggattagacagtcaaggagtggcttactacatgtgggagcttttggtatgtgacatgcgggctgggcagctgttagagtccaacgtggggcagcacagagagggggccacctccccaggccgtggctgcccacacaccccaattagctgaattcgcgtgtggcagagggaggaaaaggaggcaaacgtgggctgggcaatggcctcacataggaaacagggtcttcctggagatttggtgatggagatgtcaagcaggtggcctctggacgtcaccgttgccctgcatggtggccccagagcagcctctatgaacaacctcgtttccaaaccacagcccacagccggagagtccaggaagacttgcgcactcagagcagaagggtaggagtcctctagacagcctcgcagccgcgccagtcgcccatagacactggctgtgaccgggcgtgctggcagcggcagtgcacagtggccagcactaaccctccctgagaagataaccggctcattcacttcctcccagaagacgcgtggtagcgagtaggcacaggcgtgcacctgctcccgaattactcaccgagacacacgggctgagcagacggccccgtggatggagacaaagagctcttctgaccatatccttcttaacacccgctggcatctcctttcgcgcctccctccctaacctactgacccaccttttgattttagcgcacctgtgattgataggccttccaaagagtcccacgctggcatcaccctccccgaggacggagatgaggagtagtcagcgtgatgccaaaacgcgtcttcttaatccaattctaattctgaatgtttcgtgtgggcttaataccatgtctattaatatatagcctcgatgatgagagagttacaaagaacaaaactccagacacaaacctccaaatttttcagcagaagcactctgcgtcgctgagctgaggtcggctctgcgatccatacgtggccgcacccacacagcacgtgctgtgacgatggctgaacggaaagtgtacactgttcctgaatattgaaataaaacaataaacttttaatggta

SEQ ID NO: 30 - RNA, 5501 nt, Homo sapiens
  source 1..5501: mol_type = mRNA
agagccagagcaggatggagaggagacgcatcacctccgctgctcgccgctcctacgtctcctcaggggagatgatggtggggggcctggctcctggccgccgtctgggtcctggcacccgcctctccctggctcgaatgccccctccactcccgacccgggtggatttctccctggctggggcactcaatgctggcttcaaggagacccgggccagtgagcgggcagagatgatggagctcaatgaccgctttgccagctacatcgagaaggttcgcttcctggaacagcaaaacaaggcgctggctgctgagctgaaccagctgcgggccaaggagcccaccaagctggcagacgtctaccaggctgagctgcgagagctgcggctgcggctcgatcaactcaccgccaacagcgcccggctggaggttgagagggacaatctggcacaggacctggccactgtgaggcagaagctccaggatgaaaccaacctgaggctggaagccgagaacaacctggctgcctatagacaggaagcagatgaagccaccctggcccgtctggatctggagaggaagattgagtcgctggaggaggagatccggttcttgaggaagatccacgaggaggaggttcgggaactccaggagcagctggcccgacagcaggtccatgtggagcttgacgtggccaagccagacctcaccgcagccctgaaagagatccgcacgcagtatgaggcaatggcgtccagcaacatgcatgaagccgaagagtggtaccgctccaagtttgcagacctgacagacgctgctgcccgcaacgcggagctgctccgccaggccaagcacgaagccaacgactaccggcgccagttgcagtccttgacctgcgacctggagtctctgcgcggcacgaacgagtccctggagaggcagatgcgcgagcaggaggagcggcacgtgcgggaggcggccagttatcaggaggcgctggcgcggctggaggaagaggggcagagcctcaaggacgagatggcccgccacttgcaggagtaccaggacctgctcaatgtcaagctggccctggacatcgagatcgccacctacaggaagctgctagagggcgaggagaaccggatcaccattcccgtgcagaccttctccaacctgcagattcgagaaaccagcctggacaccaagtctgtgtcagaaggccacctcaagaggaacatcgtggtgaagaccgtggagatgcgggatggagaggtcattaaggagtccaagcaggagcacaaggatgtgatgtgaggcaggacccacctggtggcctctgccccgtctcatgaggggcccgagcagaagcaggatagttgctccgcctctgctggcacatttccccagacctgagctccccaccaccccagctgctcccctccctcctctgtccctaggtcagcttgctgccctaggctccgtcagtatcaggcctgccagacggcacccacccagcacccagcaactccaactaacaagaaactcacccccaaggggcagtctggaggggcatggccagcagcttgcgttagaatgaggaggaaggagagaaggggaggagggcggggggcacctactacatcgccctccacatccctgattcctgttgttatggaaactgttgccagagatggaggttctctcggagtatctgggaactgtgcctttgagtttcctcaggctgctggaggaaaactgagactcagacaggaaagggaaggccccacagacaaggtagccctggccagaggcttgttttgtcttttggtttttatgaggtgggatatccctatgctgcctaggctgaccttgaactcctgggctcaagcagtctacccacctcagcctcctgtgtagctgggattatagattggagccaccatgcccagctcagagggttgttctcctagactgaccctgatcagtctaagatgggtggggacgtcctgccacctggggcagtcacctgcccagatcccagaaggacctcctgagcgatgactcaagtgtctcagtccacctgagctgccatccagggatgccatctgtgggcacgctgtgggcaggtgggagcttgattctcagcacttgggggatctgttgtgtacgtggagagggatgaggtgctgggagggatagaggggggctgcctggcccccagctgtgggtacagagaggtcaagcccaggaggactgccccgtgcagactggaggggacgctggtagagatggaggaggaggcaattgggatggcgctaggcatacaagtaggggttgtgggtgaccagttgcacttggcctctggattgtgggaattaaggaagtgactcatcctcttgaagatgctgaaacaggagagaaaggggatgtatccatgggggcagggcatgactttgtcccatttctaaaggcctcttccttgctgtgtcataccaggccgccccagcctctgagcccctgggactgctgcttcttaaccccagtaagccactgccacacgtctgaccctctccaccccatagtgaccggctgcttttccctaagccaagggcctcttgcggtcccttcttactcacacacaaaatgtacccagtattctaggtagtgccctattttacaattgtaaaactgaggcacgagcaaagtgaagacactggctcatattcctgcagcctggaggccgggtgctcagggctgacacgtccaccccagtgcacccactctgctttgactgagcagactggtgagcagactggtgggatctgtgcccagagatgggactgggagggcccacttcagggttctcctctcccctctaaggccgaagaagggtccttccctctccccaagacttggtgtcctttccctccactccttcctgccacctgctgctgctgctgctgctaatcttcagggcactgctgctgcctttagtcgctgaggaaaaataaagacaaatgctgcgcccttccccagagtggactctgatctgttcatgagagggcgggactggggccaagatgtagcctttgacaagaccaactcatttcttattactgatcatctctggggcccatgccctcaccaaattccacccgcagccaaagaggacatacaccagctccctccactcttttcttccttcctctccctgctacctgcaactcaaccagcacaatcttcataggcaagaaagcaaagcagctcaaacatgattcaacactgatcagtgtttaccactggataaatctgagttcacactttccttctctgacctaaatgtgaagtcaggaaacacatgtgccctacttccatcctgagctcagtccccaatctcccaccagcctcaggcccctccacttctcagatcaggtcccagacctgcccatgaaaatggggagcaggctgtaacagatttgtccacatgttcctaccacctgtcccaacccagggtacccacccagagacatctggtatcatttaacaaacacattgaaggacaactggtcttcagagctgaagagagctcctagggggagaagctgggacaacagtgaaataagtagcagcagcaacgacagaagtgaatggtgacaaagactgctgtgatgagcaggtagcctatcagggtgagctccacagccgagcgagtctcaggatctgagaacgaggctgggtagtgcccatgagatgtcacacccagccggaagccagcaactagcacaccctgcctccagcaatagtagatgccccggtcatccagctgggtgaagcggatgtggagctggttgccgtggtcaatgaacaccctcatggacctgttgacacccttcaggtactgtgtgcggtagaggtgctggcggtctttgtcccaggccactgcatgctctggccgggccccaggacaggagatgatgagtccatggcccagtctctgctggtggaactgaatgggcacctggggcacccagggccggctgcccactttggacacatagttaatgatggccagcacgccctcccggatggtctttgtcttctcacagggtactaagcagctccgaaccagcacctcaggcgtgtggtccctggccttggtccgcagcttccttggcacagcccttgagccacaagacaccacatcgggcacggccttgaggtagcgtggggagaggtctgggctctgcaggtagcagaggccgatgcgccactgctccccacgcactccgcagcggtcacagggggtccattcccagaaggtggtgaagacatggaggtgcccatagtattcatctgcaaagggctcctggcccttgtcctggaaagtggccaccattccctcactgttctggatgtccacatcgtaggcgtaaaagtagtcccccttgcgggtgccgcagaagtacaggcctgagtcctcagactgagccctgaaaaccaacaagctgaacatgcggatgctgaagcgggtcagcatgtcgctgcccacacgtacctgggctgcctccgtcagcacccgcccatcaaagtccgtcagcactttggtgtggctgctacctaggtgcttttggtagaaccagactacagctggcacctcttcgggtttgcagtgacagggaagctcaaagctcatgtcggccaggtaggctgcattttcaaacatcaggaaagcagggcagggggtcctctgaaaaatgttttccttctccacaatttcaaaggcctggagcccccatgcccacaggagcacagtggtgagggccaggtgcatacctgaaggaggcaggggtcagaggggcagggcaaaaccagggcattaaaggctcatagggctcctagaaagctctgctaagcggaagcctctagatgaggaaaggattatgcagccaggaaaagcagcaacaatctgcagaggaagccgccaagtgcaaggcaatttattcccagtggatgtacaagatgcccttctaacattccagacctgatctcagggtggggggggaaagccattctagaacctggcctttactcccctttctagaacactggcgctcacccaagaatgggtcaaaggaaaccggaatgagaagggcgggccgaggtgctcgggcagggagatctctgcctcagtgctccaggccctgccctgccagcctggtggaaaagtctttcatcaacctgggggatgaaggaaacccaccctcctgcatatctggccatccgggaggctggctggacctgagctgatggcttgggactttcccaggcccaacctgcacaagaactgagtctctaggggaaaattcaacacctcaaatgatgtagtatttgatcatttgttgattacatgtccattcattggtttggggctataaacattcttgttaagagctgtggagatcagtgtttgtttaccataaagattttgctttttccctttta

SEQ ID NO: 31 - RNA, 1828 nt, Homo sapiens
  source 1..1828: mol_type = mRNA
agttacagggagcaccaccagggaacatctcggggagcctggttggaagctgcaggcttagtctgtcggctgcgggtctctgactgccctgtggggagggtcttgccttaacatcccttgcatttggctgcaaagaaatctgcttggaagaaggggttacgctgtttggccgggcagaaactccgctgagcagaacttgccgccagaatgctcctcctgttgctgagtatcatcgtcctccacgtcgcggtgctggtgctgctgttcgtctccacgatcgtcagccaatggatcgtgggcaatggacacgcaactgatctctggcagaactgtagcacctcttcctcaggaaatgtccaccactgtttctcatcatcaccaaacgaatggctgcagtctgtccaggccaccatgatcctgtcgatcatcttcagcattctgtctctgttcctgttcttctgccaactcttcaccctcaccaaggggggcaggttttacatcactggaatcttccaaattcttgctggtctgtgcgtgatgagtgctgcggccatctacacggtgaggcacccggagtggcatctcaactcggattactcctacggtttcgcctacatcctggcctgggtggccttccccctggcccttctcagcggtgtcatctatgtgatcttgcggaaacgcgaatgaggcgcccagacggtctgtctgaggctctgagcgtacatagggaagggaggaagggaaaacagaaagcagacaaagaaaaaagagctagcccaaaatcccaaactcaaaccaaaccaaacagaaagcagtggaggtgggggttgctgttgattgaagatgtatataatatctccggtttataaaacctatttataacactttttacatatatgtacatagtattgtttgctttttatgttgaccatcagcctcgtgttgagccttaaagaagtagctaaggaactttacatcctaacagtataatccagctcagtatttttgttttgttttttgtttgtttgttttgttttacccagaaataagataactccatctcgccccttccctttcatctgaaagaagatacctccctcccagtccacctcatttagaaaaccaaagtgtgggtagaaaccccaaatgtccaaaagcccttttctggtgggtgacccagtgcatccaacagaaacagccgctgcccgaacctctgtgtgaagctttacgcgcacacggacaaaatgcccaaactggagcccttgcaaaaacacggcttgtggcattggcatacttgcccttacaggtggagtatcttcgtcacacatctaaatgagaaatcagtgacaacaagtctttgaaatggtgctatggatttaccattccttattatcactaatcatctaaacaactcactggaaatccaattaacaattttacaacataagatagaatggagacctgaataattctgtgtaatataaatggtttataactgcttttgtacctagctaggctgctattattactataatgagtaaatcataaagccttcatcactcccacatttttcttacggtcggagcatcagaacaagcgtctagactccttgggaccgtgagttcctagagcttggctgggtctaggctgttctgtgcctccaaggactgtctggcaatgacttgtattggccaccaactgtagatgtatatatggtgcccttctgatgctaagactccagaccttttgtttttgctttgcattttctgattttataccaactgtgtggactaagatgcattaaaataaacatcagagtaactca

SEQ ID NO: 32 - RNA, 3252 nt, Homo sapiens
  source 1..3252: mol_type = mRNA
actcacaggttgggtttgatcgcatgcgtgtcggagaggagagagcagagagagacacaggaacaagaacagcaaagggtagagcagacctgcgccaggggcgcacaacggccgtgtccacctcccggccccaagatggtgcttcccacaggcagccacgcgtagcagccagagacagctccagacatgtggctcttcttcgggatcactggattgctgacggcagccctctcaggtcatccatctccagccccacctgaccagctcaacaccagcagtgcagagagtgagctctgggagccaggggagcggcttccggtccgtctgacaaacgggagcagcagctgcagcgggacggtggaggtgcggctcgaggcgtcctgggagcccgcgtgcggggcgctctgggacagccgcgccgccgaggccgtgtgccgagcactgggctgcggcggggcggaggccgcctctcagctcgccccgccgacccctgagctgccgcccccgcctgcagccgggaacaccagcgtagcagctaatgccactctggccggggcgcccgccctcctgtgcagcggcgccgagtggcggctctgcgaggtggtggagcacgcgtgccgcagcgacgggaggcgggcccgtgtcacctgtgcagagaaccgcgcgctgcgcctggtggacggtggcggcgcctgcgccggccgcgtggagatgctggagcatggcgagtggggatcagtgtgcgatgacacttgggacctggaggacgcccacgtggtgtgcaggcaactgggctgcggctgggcagtccaggccctgcccggcttgcacttcacgcccggccgcgggcctatccaccgggaccaggtgaactgctcgggggccgaagcttacctgtgggactgcccggggctgccaggacagcactactgcggccacaaagaggacgcgggcgcggtgtgctcagagcaccagtcctggcgcctgacagggggcgctgaccgctgcgaggggcaggtggaggtacacttccgaggggtctggaacacagtgtgtgacagtgagtggtacccatcggaggccaaggtgctctgccagtccttgggctgtggaactgcggttgagaggcccaaggggctgccccactccttgtccggcaggatgtactactcatgcaatggggaggagctcaccctctccaactgctcctggcggttcaacaactccaacctctgcagccagtcgctggcagccagggtcctctgctcagcttcccggagtttgcacaatctgtccactcccgaagtccctgcaagtgttcagacagtcactatagaatcttctgtgacagtgaaaatagagaacaaggaatctcgggagctaatgctcctcatcccctccatcgttctgggaattctcctccttggctccctcatcttcatagccttcatcctcttgagaattaaaggaaaatatgccctccccgtaatggtgaaccaccagcacctacccaccaccatcccggcagggagcaatagctatcaaccggtccccatcaccatccccaaagaagttttcatgctgcccatccaggtccaggccccgccccctgaggactcagactctggctcggactcagactatgagcactatgacttcagcgcccagcctcctgtggccctgaccaccttctacaattcccagcggcatcgggtcacagatgaggaggtccagcaaagcaggttccagatgccacccttggaggaaggacttgaagagttgcatgcctcccacatcccaactgccaaccctggacactgcattacagacccgccatccctgggccctcagtatcacccgaggagcaacagtgagtcgagcacctcttcaggggaggattactgcaatagtcccaaaagcaagctgcctccatggaacccccaggtgttttcttcagagaggagttccttcctggagcagcccccaaacttggagctggccggcacccagccagccttttcagcagggcccccggctgatgacagctccagcacctcatccggggagtggtaccagaacttccagccaccaccccagcccccttcggaggagcagtttggctgtccagggtcccccagccctcagcctgactccaccgacaacgatgactacgatgacatcagcgcagcctaggccggggccagccgaggctcctggggtggctctgaccctctggcctcctgctctacctactccctttcccctttcccaccctcccagctcacctccccatggagctgagaggcctcccttggagagatggaaggaaacgttataccttgtacccctcggtctccatccatcaagccaaacctgctgccacagccctcccccggccccagatagcagccccagggaggatgctgcctccaagaggtgtgagccctctgtctcggggatgaacaagcagagtctgggctacctcttgacagctggtggaggggagttggggagctggactggatgactctggaggccccttccaaacctcaagtgtccggcgctttgattgcctgagtttctgacacttcagggcccagaggtcctgcgaggggcagaactggacccccatgccagtgctgctgcaggagggcccatatactagggtctgctgagctgttgtcactgatcggtgggcgctgggggggtagggtagcacaccagctgtcccaggctttgctccgggcggtaactgcacttgggcagggaatatagccttcctgggcacaactagctgacaatgacaggttgactgtgtacccccaaccaaggagctggggcccaaggccagtcctgccccagagacactccaagtccgccaggggcacagaccagttctgcagtgactgtccctggacaatgggtctttattctgagtttcctatggtttacaaagagggccccagcccagccccaccacagatcccagagataggggcccagtctccatgggggcaaggagcatagagatgttttccaggaaggggctcagaagctgcactaggccccgagtccccatgtgtctccttgaattgatgaggatgctcctgggagggatgcgtgactatgtggtgttgcacccggggctgcaaacgtctccgtgcagcccccagagagaggcccatgggctcagaccaggctttgttgtcctgctctgagtatcctgagattaaactgaattgctgaatgaaa

SEQ ID NO: 33 - RNA, 1225 nt, Homo sapiens
  source 1..1225: mol_type = mRNA
agccgcctgcatctgtatccagcgccaggtcccgccagtcccagctgcgcgcgccccccagtcccgcacccgttcggcccaggctaagttagccctcaccatgccggtcaaaggaggcaccaagtgcatcaaatacctgctgttcggatttaacttcatcttctggcttgccgggattgctgtccttgccattggactatggctccgattcgactctcagaccaagagcatcttcgagcaagaaactaataataataattccagcttctacacaggagtctatattctgatcggagccggcgccctcatgatgctggtgggcttcctgggctgctgcggggctgtgcaggagtcccagtgcatgctgggactgttcttcggcttcctcttggtgatattcgccattgaaatagctgcggccatctggggatattcccacaaggatgaggtgattaaggaagtccaggagttttacaaggacacctacaacaagctgaaaaccaaggatgagccccagcgggaaacgctgaaagccatccactatgcgttgaactgctgtggtttggctgggggcgtggaacagtttatctcagacatctgccccaagaaggacgtactcgaaaccttcaccgtgaagtcctgtcctgatgccatcaaagaggtcttcgacaataaattccacatcatcggcgcagtgggcatcggcattgccgtggtcatgatatttggcatgatcttcagtatgatcttgtgctgtgctatccgcaggaaccgcgagatggtctagagtcagcttacatccctgagcaggaaagtttacccatgaagattggtgggattttttgtttgtttgttttgttttgtttgttgtttgttgtttgtttttttgccactaattttagtattcattctgcattgctagataaaagctgaagttactttatgtttgtcttttaatgcttcattcaatattgacatttgtagttgagcggggggtttggtttgctttggtttatattttttcagttgtttgtttttgcttgttatattaagcagaaatcctgcaatgaaaggtactatatttgctagactctagacaagatattgtacataaaagaatttttttgtctttaaatagatacaaatgtctatcaactttaatcaagttgtaacttatattgaagacaatttgatacataataaaaaattatgacaatgtcctgga

SEQ ID NO: 34 - RNA, 5431 nt, Homo sapiens
  source 1..5431: mol_type = mRNA
ctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccactttgatgagcactagtgctacagcaactgagacagcaaccaagaggcaagaaacctgggattggttttcatggttgtttctaccatcagagtcaaagaatcatcttcacacaacaacacaaatggctggtacgtcttcaaataccatctcagcaggctgggagccaaatgaagaaaatgaagatgaaagagacagacacctcagtttttctggatcaggcattgatgatgatgaagattttatctccagcaccatttcaaccacaccacgggcttttgaccacacaaaacagaaccaggactggacccagtggaacccaagccattcaaatccggaagtgctacttcagacaaccacaaggatgactgatgtagacagaaatggcaccactgcttatgaaggaaactggaacccagaagcacaccctcccctcattcaccatgagcatcatgaggaagaagagaccccacattctacaagcacaatccaggcaactcctagtagtacaacggaagaaacagctacccagaaggaacagtggtttggcaacagatggcatgagggatatcgccaaacacccaaagaagactcccattcgacaacagggacagctgcagcctcagctcataccagccatccaatgcaaggaaggacaacaccaagcccagaggacagttcctggactgatttcttcaacccaatctcacaccccatgggacgaggtcatcaagcaggaagaaggatggatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaacaga

SEQ ID NO: 35 - RNA, 3402 nt, Homo sapiens
  source 1..3402: mol_type = mRNA
cacttccgccccgggcgcggctcgggccacgcccacctgtcctgcagcactggatgctttgtgagttggggattgttgcgtcccatatctggacccagaagggacttccctgctcggctggctctcggtttctctgctttcctccggagaaataacagcgtcttccgcgccgcgcatggagcctcccggccgccgcgagtgtccctttccttcctggcgctttcctgggttgcttctggcggccatggtgttgctgctgtactccttctccgatgcctgtgaggagccaccaacatttgaagctatggagctcattggtaaaccaaaaccctactatgagattggtgaacgagtagattataagtgtaaaaaaggatacttctatatacctcctcttgccacccatactatttgtgatcggaatcatacatggctacctgtctcagatgacgcctgttatagagaaacatgtccatatatacgggatcctttaaatggccaagcagtccctgcaaatgggacttacgagtttggttatcagatgcactttatttgtaatgagggttattacttaattggtgaagaaattctatattgtgaacttaaaggatcagtagcaatttggagcggtaagcccccaatatgtgaaaaggttttgtgtacaccacctccaaaaataaaaaatggaaaacacacctttagtgaagtagaagtatttgagtatcttgatgcagtaacttatagttgtgatcctgcacctggaccagatccattttcacttattggagagagcacgatttattgtggtgacaattcagtgtggagtcgtgctgctccagagtgtaaagtggtcaaatgtcgatttccagtagtcgaaaatggaaaacagatatcaggatttggaaaaaaattttactacaaagcaacagttatgtttgaatgcgataagggtttttacctcgatggcagcgacacaattgtctgtgacagtaacagtacttgggatcccccagttccaaagtgtcttaaagtgctgcctccatctagtacaaaacctccagctttgagtcattcagtgtcgacttcttccactacaaaatctccagcgtccagtgcctcaggtcctaggcctacttacaagcctccagtctcaaattatccaggatatcctaaacctgaggaaggaatacttgacagtttggatgtttgggtcattgctgtgattgttattgccatagttgttggagttgcagtaatttgtgttgtcccgtacagatatcttcaaaggaggaagaagaaaggcacatacctaactgatgagacccacagagaagtaaaatttacttctctctgagaaggagagatgagagaaaggtttgcttttatcattaaaaggaaagcagatggtggagctgaatatgccacttaccagactaaatcaaccactccagcagagcagagaggctgaatagattccacaacctggtttgccagttcatcttttgactctattaaaatcttcaatagttgttattctgtagtttcactctcatgagtgcaactgtggcttagctaatattgcaatgtggcttgaatgtaggtagcatcctttgatgcttctttgaaacttgtatgaatttgggtatgaacagattgcctgctttcccttaaataacacttagatttattggaccagtcagcacagcatgcctggttgtattaaagcagggatatgctgtattttataaaattggcaaaattagagaaatatagttcacaatgaaattatattttctttgtaaagaaagtggcttgaaatcttttttgttcaaagattaatgccaactcttaagattattctttcaccaactatagaatgtattttatatatcgttcattgtaaaaagcccttaaaaatatgtgtatactactttggctcttgtgcataaaaacaagaacactgaaaattgggaatatgcacaaacttggcttctttaaccaagaatattattggaaaattctctaaaagttaatagggtaaattctctattttttgtaatgtgttcggtgatttcagaaagctagaaagtgtatgtgtggcatttgttttcactttttaaaacatccctaactgatcgaatatatcagtaatttcagaatcagatgcatcctttcataagaagtgagaggactctgacagccataacaggagtgccacttcatggtgcgaagtgaacactgtagtcttgttgttttcccaaagagaactccgtatgttctcttaggttgagtaacccactctgaattctggttacatgtgtttttctctccctccttaaataaagagaggggttaaacatgccctctaaaagtaggtggttttgaagagaataaattcatcagataacctcaagtcacatgagaatcttagtccatttacattgccttggctagtaaaagccatctatgtatatgtcttacctcatctcctaaaaggcagagtacaaagtaagccatgtatctcaggaaggtaacttcattttgtctatttgctgttgattgtaccaagggatggaagaagtaaatatagctcaggtagcactttatactcaggcagatctcagccctctactgagtcccttagccaagcagtttctttcaaagaagccagcaggcgaaaagcagggactgccactgcatttcatatcacactgttaaaagttgtgttttgaaattttatgtttagttgcacaaattgggccaaagaaacattgccttgaggaagatatgattggaaaatcaagagtgtagaagaataaatactgttttactgtccaaagacatgtttatagtgctctgtaaatgttcctttcctttgtagtctctggcaagatgctttaggaagataaaagtttgaggagaacaaacaggaattctgaattaagcacagagttgaagtttatacccgtttcacatgcttttcaagaatgtcgcaattactaagaagcagataatggtgttttttagaaacctaattgaagtatattcaaccaaatactttaatgtataaaataaatattatacaatatacttgtatagcagtttctgcttcacatttgattttttcaaatttaatatttatattagagatctatatatgtataaatatgtattttgtcaaatttgttacttaaatatatagagaccagttttctctggaagtttgtttaaatgacagaagcgtatatgaattcaagaaaatttaagctgcaaaaatgtatttgctataaaatgagaagtctcactgatagaggttctttattgctcattttttaaaaaatggactcttgaaatctgttaaaataaaattgtacatttggagatgtttcatgaaaaaaaaaa

SEQ ID NO: 36 - RNA, 4250 nt, Homo sapiens
  source 1..4250: mol_type = mRNA
attcgcctctgggaggtttaggaagcggctccgggtcggtggccccaggacagggaagagcgggcgctatggggagccggacgccagagtcccctctccacgccgtgcagctgcgctggggcccccggcgccgacccccgctgctgccgctgctgttgctgctgctgccgccgccacccagggtcgggggcttcaacttagacgcggaggccccagcagtactctcggggcccccgggctccttcttcggattctcagtggagttttaccggccgggaacagacggggtcagtgtgctggtgggagcacccaaggctaataccagccagccaggagtgctgcagggtggtgctgtctacctctgtccttggggtgccagccccacacagtgcacccccattgaatttgacagcaaaggctctcggctcctggagtcctcactgtccagctcagagggagaggagcctgtggagtacaagtccttgcagtggttcggggcaacagttcgagcccatggctcctccatcttggcatgcgctccactgtacagctggcgcacagagaaggagccactgagcgaccccgtgggcacctgctacctctccacagataacttcacccgaattctggagtatgcaccctgccgctcagatttcagctgggcagcaggacagggttactgccaaggaggcttcagtgccgagttcaccaagactggccgtgtggttttaggtggaccaggaagctatttctggcaaggccagatcctgtctgccactcaggagcagattgcagaatcttattaccccgagtacctgatcaacctggttcaggggcagctgcagactcgccaggccagttccatctatgatgacagctacctaggatactctgtggctgttggtgaattcagtggtgatgacacagaagactttgttgctggtgtgcccaaagggaacctcacttacggctatgtcaccatccttaatggctcagacattcgatccctctacaacttctcaggggaacagatggcctcctactttggctatgcagtggccgccacagacgtcaatggggacgggctggatgacttgctggtgggggcacccctgctcatggatcggacccctgacgggcggcctcaggaggtgggcagggtctacgtctacctgcagcacccagccggcatagagcccacgcccacccttaccctcactggccatgatgagtttggccgatttggcagctccttgacccccctgggggacctggaccaggatggctacaatgatgtggccatcggggctccctttggtggggagacccagcagggagtagtgtttgtatttcctgggggcccaggagggctgggctctaagccttcccaggttctgcagcccctgtgggcagccagccacaccccagacttctttggctctgcccttcgaggaggccgagacctggatggcaatggatatcctgatctgattgtggggtcctttggtgtggacaaggctgtggtatacaggggccgccccatcgtgtccgctagtgcctccctcaccatcttccccgccatgttcaacccagaggagcggagctgcagcttagaggggaaccctgtggcctgcatcaaccttagcttctgcctcaatgcttctggaaaacacgttgctgactccattggtttcacagtggaacttcagctggactggcagaagcagaagggaggggtacggcgggcactgttcctggcctccaggcaggcaaccctgacccagaccctgctcatccagaatggggctcgagaggattgcagagagatgaagatctacctcaggaacgagtcagaatttcgagacaaactctcgccgattcacatcgctctcaacttctccttggacccccaagccccagtggacagccacggcctcaggccagccctacattatcagagcaagagccggatagaggacaaggctcagatcttgctggactgtggagaagacaacatctgtgtgcctgacctgcagctggaagtgtttggggagcagaaccatgtgtacctgggtgacaagaatgccctgaacctcactttccatgcccagaatgtgggtgagggtggcgcctatgaggctgagcttcgggtcaccgcccctccagaggctgagtactcaggactcgtcagacacccagggaacttctccagcctgagctgtgactactttgccgtgaaccagagccgcctgctggtgtgtgacctgggcaaccccatgaaggcaggagccagtctgtggggtggccttcggtttacagtccctcatctccgggacactaagaaaaccatccagtttgacttccagatcctcagcaagaatctcaacaactcgcaaagcgacgtggtttcctttcggctctccgtggaggctcaggcccaggtcaccctgaacggtgtctccaagcctgaggcagtgctattcccagtaagcgactggcatccccgagaccagcctcagaaggaggaggacctgggacctgctgtccaccatgtctatgagctcatcaaccaaggccccagctccattagccagggtgtgctggaactcagctgtccccaggctctggaaggtcagcagctcctatatgtgaccagagttacgggactcaactgcaccaccaatcaccccattaacccaaagggcctggagttggatcccgagggttccctgcaccaccagcaaaaacgggaagctccaagccgcagctctgcttcctcgggacctcagatcctgaaatgcccggaggctgagtgtttcaggctgcgctgtgagctcgggcccctgcaccaacaagagagccaaagtctgcagttgcatttccgagtctgggccaagactttcttgcagcgggagcaccagccatttagcctgcagtgtgaggctgtgtacaaagccctgaagatgccctaccgaatcctgcctcggcagctgccccaaaaagagcgtcaggtggccacagctgtgcaatggaccaaggcagaaggcagctatggcgtcccactgtggatcatcatcctagccatcctgtttggcctcctgctcctaggtctactcatctacatcctctacaagcttggattcttcaaacgctccctcccatatggcaccgccatggaaaaagctcagctcaagcctccagccacctctgatgcctgagtcctcccaatttcagactcccattcctgaagaaccagtccccccaccctcattctactgaaaaggaggggtctgggtacttcttgaaggtgctgacggccagggagaagctcctctccccagcccagagacatacttgaagggccagagccaggggggtgaggagctggggatccctcccccccatgcactgtgaaggacccttgtttacacataccctcttcatggatgggggaactcagatccagggacagaggccccagcctccctgaagcctttgcattttggagagtttcctgaaacaacttggaaagataactaggaaatccattcacagttctttgggccagacatgccacaaggacttcctgtccagctccaacctgcaaagatctgtcctcagccttgccagagatccaaaagaagcccccagctaagaacctggaacttggggagttaagacctggcagctctggacagccccaccctggtgggccaacaaagaacactaactatgcatggtgccccaggaccagctcaggacagatgccacacaaggatagatgctggcccagggcccagagcccagctccaaggggaatcagaactcaaatggggccagatccagcctggggtctggagttgatctggaacccagactcagacattggcacctaatccaggcagatccaggactatatttgggcctgctccagacctgatcctggaggcccagttcaccctgatttaggagaagccaggaatttcccaggaccctgaaggggccatgatggcaacagatctggaacctcagcctggccagacacaggccctccctgttccccagagaaaggggagcccactgtcctgggcctgcagaatttgggttctgcctgccagctgcactgatgctgcccctcatctctctgcccaacccttccctcaccttggcaccagacacccaggacttatttaaactctgttgcaagtgcaataaatctgacccagtgcccccactgaccagaactagaa

SEQ ID NO: 37 - RNA, 1482 nt, Homo sapiens
  source 1..1482: mol_type = mRNA
ggccagagagcgagcgcgcaacggcggcgacggcggcgaccccaccgcgcatcctgccaggcctccggcgcccagcgccccacgcgcccccgcgcccccgcgcccccgcgcccctttcttcgcgcccccgcccctcggcccgccaggcccccttgccggccacccgccaggccccgcgccggcccgcccgccgcccaggaccggcccgcgccccgcaggccgcccgccgcccgcgccgccatgggagtggagggctgcaccaagtgcatcaagtacctgctcttcgtcttcaatttcgtcttctggctggctggaggcgtgatcctgggtgtggccctgtggctccgccatgacccgcagaccaccaacctcctgtatctggagctgggagacaagcccgcgcccaacaccttctatgtaggcatctacatcctcatcgctgtgggcgctgtcatgatgttcgttggcttcctgggctgctacggggccatccaggaatcccagtgcctgctggggacgttcttcacctgcctggtcatcctgtttgcctgtgaggtggccgccggcatctggggctttgtcaacaaggaccagatcgccaaggatgtgaagcagttctatgaccaggccctacagcaggccgtggtggatgatgacgccaacaacgccaaggctgtggtgaagaccttccacgagacgcttgactgctgtggctccagcacactgactgctttgaccacctcagtgctcaagaacaatttgtgtccctcgggcagcaacatcatcagcaacctcttcaaggaggactgccaccagaagatcgatgacctcttctccgggaagctgtacctcatcggcattgctgccatcgtggtcgctgtgatcatgatcttcgagatgatcctgagcatggtgctgtgctgtggcatccggaacagctccgtgtactgaggccccgcagctctggccacagggacctctgcagtgccccctaagtgacccggacacttccgagggggccatcaccgcctgtgtatataacgtttccggtattactctgctacacgtagcctttttacttttggggttttgtttttgttctgaactttcctgttaccttttcagggctgacgtcacatgtaggtggcgtgtatgagtggagacgggcctgggtcttggggactggagggcaggggtccttctgccctggggtcccagggtgctctgcctgctcagccaggcctctcctgggagccactcgcccagagactcagcttggccaacttggggggctgtgtccacccagcccgcccgtcctgtgggctgcacagctcaccttgttccctcctgccccggttcgagagccgagtctgtgggcactctctgccttcatgcacctgtcctttctaacacgtcgccttcaactgtaatcacaacatcctgactccgtcatttaataaagaaggaacatcaggcatgcta

SEQ ID NO: 38 - RNA, 3327 nt, Homo sapiens
  source 1..3327: mol_type = mRNA
acttggctctcgccctccggccgggaagcatggggcttcccaggctggtctgcgccttcttgctcgccgcctgctgctgctgtcctcgcgtcgcgggtgtgcccggagaggctgagcagcctgcgcctgagctggtggaggtggaagtgggcagcacagcccttctgaagtgcggcctctcccagtcccaaggcaacctcagccatgtcgactggttttctgtccacaaggagaagcggacgctcatcttccgtgtgcgccagggccagggccagagcgaacctggggagtacgagcagcggctcagcctccaggacagaggggctactctggccctgactcaagtcaccccccaagacgagcgcatcttcttgtgccagggcaagcgccctcggtcccaggagtaccgcatccagctccgcgtctacaaagctccggaggagccaaacatccaggtcaaccccctgggcatccctgtgaacagtaaggagcctgaggaggtcgctacctgtgtagggaggaacgggtaccccattcctcaagtcatctggtacaagaatggccggcctctgaaggaggagaagaaccgggtccacattcagtcgtcccagactgtggagtcgagtggtttgtacaccttgcagagtattctgaaggcacagctggttaaagaagacaaagatgcccagttttactgtgagctcaactaccggctgcccagtgggaaccacatgaaggagtccagggaagtcaccgtccctgttttctacccgacagaaaaagtgtggctggaagtggagcccgtgggaatgctgaaggaaggggaccgcgtggaaatcaggtgtttggctgatggcaaccctccaccacacttcagcatcagcaagcagaaccccagcaccagggaggcagaggaagagacaaccaacgacaacggggtcctggtgctggagcctgcccggaaggaacacagtgggcgctatgaatgtcagggcctggacttggacaccatgatatcgctgctgagtgaaccacaggaactactggtgaactatgtgtctgacgtccgagtgagtcccgcagcccctgagagacaggaaggcagcagcctcaccctgacctgtgaggcagagagtagccaggacctcgagttccagtggctgagagaagagacaggccaggtgctggaaagggggcctgtgcttcagttgcatgacctgaaacgggaggcaggaggcggctatcgctgcgtggcgtctgtgcccagcatacccggcctgaaccgcacacagctggtcaacgtggccatttttggccccccttggatggcattcaaggagaggaaggtgtgggtgaaagagaatatggtgttgaatctgtcttgtgaagcgtcagggcacccccggcccaccatctcctggaacgtcaacggcacggcaagtgaacaagaccaagatccacagcgagtcctgagcaccctgaatgtcctcgtgaccccggagctgttggagacaggtgttgaatgcacggcctccaacgacctgggcaaaaacaccagcatcctcttcctggagctggtcaatttaaccaccctcacaccagactccaacacaaccactggcctcagcacttccactgccagtcctcataccagagccaacagcacctccacagagagaaagctgccggagccggagagccggggcgtggtcatcgtggctgtgattgtgtgcatcctggtcctggcggtgctgggcgctgtcctctatttcctctataagaagggcaagctgccgtgcaggcgctcagggaagcaggagatcacgctacccccgtctcgtaagagcgaacttgtagttgaagttaagtcagataagctcccagaagagatgggcctcctgcagggcagcagcggtgacaagagggctccgggagaccagggagagaaatacatcgatctgaggcattagccccgaatcacttcagctcccttccctgcctggaccattcccagctccctgctcactcttctctcagccaaagcctccaaagggactagagagaagcctcctgctcccctcgcctgcacaccccctttcagagggccactgggttaggacctgaggacctcacttggccctgcaaggcccgcttttcagggaccagtccaccaccatctcctccacgttgagtgaagctcatcccaagcaaggagccccagtctcccgagcgggtaggagagtttcttgcagaacgtgttttttctttacacacattatggctgtaaatacctggctcctgccagcagctgagctgggtagcctctctgagctggtttcctgccccaaaggctggcttccaccatccaggtgcaccactgaagtgaggacacaccggagccaggcgcctgctcatgttgaagtgcgctgttcacacccgctccggagagcaccccagcagcatccagaagcagctgcagtgttgctgccaccaccctcctgtctgcctcttcaaagtctcctgtgacattttttctttggtcagaagccaggaactggtgtcattccttaaaagatacgtgccggggccaggtgtggtggctcacgcctgtaatcccagcactttgggaggccgaggcgggcggatcacaaagtcaggacgagaccatcctggctaacacggtgaaaccctgtctctactaaaaatacaaaaaaaaattagctaggcgtagtggttggcacctatagtcccagctactcggaaggctgaagcaggagaatggtatgaatccaggaggtggagcttgcagtgagccgagaccgtgccactgcactccagcctgggcaacacagcgagactccgtctcgaggaaaaaaaaagaaaagatacgtgcctgcggtgaggaagctgggcgctgttttcgagttcaggtgaattagcctcaatcccccgtgttcacttggctcccatagccctcttgatggatcacgtaaaactgaaaggcagcggggagcagacaaagatgaggtctacactgtccttcatggggattaaagctatggttatattagcaccaaacttctacaaaccaagctcagggccccaaccctagaagggcccaaatgagagaatggtacttagggatggaaaacgggcctggctagagcttcgggtgtgtgtgtctgtctgtgtgtatgcatacatatgtgtgtatatatggttttgtcaggtgtgtaaatttgcaaattgtttcctttatatatgtatgtatatatatatatgaaaatatatatatatatgaaaaataaagcttaattgtcccagaaa

SEQ ID NO: 39 - RNA, 972 nt, Homo sapiens
  source 1..972: mol_type = mRNA
agcggttggaggttgtaggaccggcgaggaataggaatcatggcggctgcgctgttcgtgctgctgggattcgcgctgctgggcacccacggagcctccggggctgccggcacagtcttcactaccgtagaagaccttggctccaagatactcctcacctgctccttgaatgacagcgccacagaggtcacagggcaccgctggctgaaggggggcgtggtgctgaaggaggacgcgctgcccggccagaaaacggagttcaaggtggactccgacgaccagtggggagagtactcctgcgtcttcctccccgagcccatgggcacggccaacatccagctccacgggcctcccagagtgaaggctgtgaagtcgtcagaacacatcaacgagggggagacggccatgctggtctgcaagtcagagtccgtgccacctgtcactgactgggcctggtacaagatcactgactctgaggacaaggccctcatgaacggctccgagagcaggttcttcgtgagttcctcgcagggccggtcagagctacacattgagaacctgaacatggaggccgaccccggccagtaccggtgcaacggcaccagctccaagggctccgaccaggccatcatcacgctccgcgtgcgcagccacctggccgccctctggcccctcctgggcatcgtggctgaggtgctggtgctggtcaccatcatcttcatctacgagaagcgccggaagcccgaggacgtcctggatgatgacgacgccggctctgcacccctgaagagcagcgggcagcaccagaatgacaaaggcaagaacgtccgccagaggaactcttcctgaggcaggtggcccgaggacgctccctgctccacgtctgcgccgccgccggagtccactcccagtgcttgcaagattccaagttctcacctcttaaagaaaacccaccccgtagattcccatcat

SEQ ID NO: 40 - RNA, 4662 nt, Homo sapiens
  source 1..4662: mol_type = mRNA
agagcagcccggagaccgctgccgccgctgccgctgctaccaccgctgccacctgaggagacccgccgcccccccgtcgccgcctcctgcgagtccttcttagcacctggcgtttcatgcacattgccactgccattattattatcattccaatacaaggaaaataaaagaagataccagcgaaaagaaccgcttacacctttccgaattactcaagtgtctcctggaaacagagggtcgttgtccccggaggagcagccgaagggcccgtgggctggtgttgaccgggagggaggaggagttgggggcattgcgtggtggaaagttgcgtgcggcagagaaccgaaggtgcagcgccacagcccaggggacggtgtgtctgggagaagacgctgcccctgcgtcgggacccgccagcgcgcgggcaccgcggggcccgggacgacgccccctcctgcggcgtggactccgtcagtggcccaccaagaaggaggaggaatatggaatccaagggggccagttcctgccgtctgctcttctgcctcttgatctccgccaccgtcttcaggccaggccttggatggtatactgtaaattcagcatatggagataccattatcataccttgccgacttgacgtacctcagaatctcatgtttggcaaatggaaatatgaaaagcccgatggctccccagtatttattgccttcagatcctctacaaagaaaagtgtgcagtacgacgatgtaccagaatacaaagacagattgaacctctcagaaaactacactttgtctatcagtaatgcaaggatcagtgatgaaaagagatttgtgtgcatgctagtaactgaggacaacgtgtttgaggcacctacaatagtcaaggtgttcaagcaaccatctaaacctgaaattgtaagcaaagcactgtttctcgaaacagagcagctaaaaaagttgggtgactgcatttcagaagacagttatccagatggcaatatcacatggtacaggaatggaaaagtgctacatccccttgaaggagcggtggtcataatttttaaaaaggaaatggacccagtgactcagctctataccatgacttccaccctggagtacaagacaaccaaggctgacatacaaatgccattcacctgctcggtgacatattatggaccatctggccagaaaacaattcattctgaacaggcagtatttgatatttactatcctacagagcaggtgacaatacaagtgctgccaccaaaaaatgccatcaaagaaggggataacatcactcttaaatgcttagggaatggcaaccctcccccagaggaatttttgttttacttaccaggacagcccgaaggaataagaagctcaaatacttacacactgacggatgtgaggcgcaatgcaacaggagactacaagtgttccctgatagacaaaaaaagcatgattgcttcaacagctatcacagttcactatttggatttgtccttaaacccaagtggagaagtgactagacagattggtgatgccctacccgtgtcatgcacaatatctgctagcaggaatgcaactgtggtatggatgaaagataacatcaggcttcgatctagcccgtcattttctagtcttcattatcaggatgctggaaactatgtctgcgaaactgctctgcaggaggttgaaggactaaagaaaagagagtcattgactctcattgtagaaggcaaacctcaaataaaaatgacaaagaaaactgatcccagtggactatctaaaacaataatctgccatgtggaaggttttccaaagccagccattcaatggacaattactggcagtggaagcgtcataaaccaaacagaggaatctccttatattaatggcaggtattatagtaaaattatcatttcccctgaagagaatgttacattaacttgcacagcagaaaaccaactggagagaacagtaaactccttgaatgtctctgctaatgaaaacagagaaaaggtgaatgaccaggcaaaactaattgtgggaatcgttgttggtctcctccttgctgcccttgttgctggtgtcgtctactggctgtacatgaagaagtcaaagactgcatcaaaacatgtaaacaaggacctcggtaatatggaagaaaacaaaaagttagaagaaaacaatcacaaaactgaagcctaagagagaaactgtcctagttgtccagagataaaaatcatatagaccaattgaagcatgaacgtggattgtatttaagacataaacaaagacattgacagcaattcatggttcaagtattaagcagttcattctaccaagctgtcacaggttttcagagaattatctcaagtaaaacaaatgaaatttaattacaaacaataagaacaagttttggcagccatgataataggtcatatgttgtgtttggttcaattttttttccgtaaatgtctgcactgaggatttctttttggtttgccttttatgtaaattttttacgtagctatttttatacactgtaagctttgttctgggagttgctgttaatctgatgtataatgtaatgtttttatttcaattgtttatatggataatctgagcaggtacatttctgattctgattgctatcagcaatgccccaaactttctcataagcacctaaaacccaaaggtggcagcttgtgaagattggggacactcatattgccctaattaaaaactgtgatttttatcacaagggaggggaggccgagagtcagactgatagacaccataggagccgactctttgatatgccaccagcgaactctcagaaataaatcacagatgcatatagacacacatacataatggtactcccaaactgacaattttacctattctgaaaaagacataaaacagaatttggtagcacttacctctacagacacctgctaataaattattttctgtcaaaagaaaaaacacaagcatgtgtgagagacagtttggaaaaatcatggtcaacattcccattttcatagatcacaatgtaaatcactataattacaaattggtgttaaatcctttgggttatccactgccttaaaattatacctatttcatgtttaaaaagatatcaatcagaattggagtttttaacagtggtcattatcaaagctgtgttattttccacagaatatagaatatatatttttttcgtgtgtgtttttgttaactaccctacagatattgaatgcaccttgagataatttagtgtttttaactgatacataatttatcaagcagtacatgaaagtgtaataataaaatgtctatgtatctttagttacattcaaatttgtaactttataaacatgttttatgcttgaggaaatttttaaggtggtagtataaatggaaactttttgaagtagaccagatatgggctacttgtgactagacttttaaactttgctctttcaagcagaagcctggtttctgggagaacactgcacagcgatttctttcccaggatttacacaactttaaagggaagataaatgaacatcagatttctaggtatagaactatgttattgaaaggaaaaggaaaactggtgtttgtttcttagactcatgaaataaaaaattatgaaggcaatgaaaaataaattgaaaattaaagtcagatgagaataggaataatactttgccacttctgcattatttagaaacatacgttattgtacatttgtaaaccatttactgtctgggcaatagtgactccgtttaataaaagcttccgtagtgcattggtatggattaaatgcataaaatattcttagactcgatgctgtataaaatattatgggaaaaaaagaaaatacgttattttgcctctaaacttttattgaagttttatttggcaggaaaaaaaattgaatcttggtcaacatttaaaccaaagtaaaaggggaaaaaccaaagttatttgttttgcatggctaagccattctgttatctctgtaaatactgtgatttcttttttattttctctttagaattttgttaaagaaattctaaaatttttaaacacctgctctccacaataaatcacaaacactaaaataaaattacttccatataaatattattttctcttttggtgtgggagatcaaaggtttaaagtctaacttctaagatatatttgcagaaagaagcaacatgacaatagagagagttatgctacaattatttcttggtttccacttgcaatggttaattaagtccaaaaacagctgtcagaacctcgagagcagaacatgagaaactcagagctctggaccgaaagcagaaagtttgccgggaaaaaaaaagacaacattattaccatcgattcagtgcctggataaagaggaaagcttacttgtttaatggcagccacatgcacgaagatgctaagaagaaaaagaattccaaatcctcaacttttgaggtttcggctctccaatttaactctttggcaacaggaaacaggttttgcaagttcaaggttcactccctatatgtgattataggaattgtttgtggaaatggattaacatacccgtctatgcctaaaagataataaaactgaaatatgtcttca

SEQ ID NO: 41 - RNA, 5099 nt, Homo sapiens
  source 1..5099: mol_type = mRNA
agtcactaacgtccttccgttctctcgtctctcttcccccacccttcccttcccttctcccctctcccagtgcccccactcccaactcccgccccaagccgcccaccagcccccttcccctccggccggagcctgaaccgagcccggctggctgtgctgcgcggtgccgccgggaaagatggtcgtggcgctgcggtacgtgtggcctctcctcctctgcagcccctgcctgcttatccagatccccgaggaatatgaaggacaccatgtgatggagccacctgtcatcacggaacagtctccacggcgcctggttgtcttccccacagatgacatcagcctcaagtgtgaggccagtggcaagcccgaagtgcagttccgctggacgagggatggtgtccacttcaaacccaaggaagagctgggtgtgaccgtgtaccagtcgccccactctggctccttcaccatcacgggcaacaacagcaactttgctcagaggttccagggcatctaccgctgctttgccagcaataagctgggcaccgccatgtcccatgagatccggctcatggccgagggtgcccccaagtggccaaaggagacagtgaagcccgtggaggtggaggaaggggagtcagtggttctgccttgcaaccctcccccaagtgcagagcctctccggatctactggatgaacagcaagatcttgcacatcaagcaggacgagcgggtgacgatgggccagaacggcaacctctactttgccaatgtgctcacctccgacaaccactcagactacatctgccacgcccacttcccaggcaccaggaccatcattcagaaggaacccattgacctccgggtcaaggccaccaacagcatgattgacaggaagccgcgcctgctcttccccaccaactccagcagccacctggtggccttgcaggggcagccattggtcctggagtgcatcgccgagggctttcccacgcccaccatcaaatggctgcgccccagtggccccatgccagccgaccgtgtcacctaccagaaccacaacaagaccctgcagctgctgaaagtgggcgaggaggatgatggcgagtaccgctgcctggccgagaactcactgggcagtgcccggcatgcgtactatgtcaccgtggaggctgccccgtactggctgcacaagccccagagccatctatatgggccaggagagactgcccgcctggactgccaagtccagggcaggccccaaccagaggtcacctggagaatcaacgggatccctgtggaggagctggccaaagaccagaagtaccggattcagcgtggcgccctgatcctgagcaacgtgcagcccagtgacacaatggtgacccaatgtgaggcccgcaaccggcacgggctcttgctggccaatgcctacatctacgttgtccagctgccagccaagatcctgactgcggacaatcagacgtacatggctgtccagggcagcactgcctaccttctgtgcaaggccttcggagcgcctgtgcccagtgttcagtggctggacgaggatgggacaacagtgcttcaggacgaacgcttcttcccctatgccaatgggaccctgggcattcgagacctccaggccaatgacaccggacgctacttctgcctggctgccaatgaccaaaacaatgttaccatcatggctaacctgaaggttaaagatgcaactcagatcactcaggggccccgcagcacaatcgagaagaaaggttccagggtgaccttcacgtgccaggcctcctttgacccctccttgcagcccagcatcacctggcgtggggacggtcgagacctccaggagcttggggacagtgacaagtacttcatagaggatgggcgcctggtcatccacagcctggactacagcgaccagggcaactacagctgcgtggccagtaccgaactggatgtggtggagagtagggcacagctcttggtggtggggagccctgggccggtgccacggctggtgctgtccgacctgcacctgctgacgcagagccaggtgcgcgtgtcctggagtcctgcagaagaccacaatgcccccattgagaaatatgacattgaatttgaggacaaggaaatggcgcctgaaaaatggtacagtctgggcaaggttccagggaaccagacctctaccaccctcaagctgtcgccctatgtccactacacctttagggttactgccataaacaaatatggccccggggagcccagcccggtctctgagactgtggtcacacctgaggcagccccagagaagaaccctgtggatgtgaagggggaaggaaatgagaccaccaatatggtcatcacgtggaagccgctccggtggatggactggaacgccccccaggttcagtaccgcgtgcagtggcgccctcaggggacacgagggccctggcaggagcagattgtcagcgaccccttcctggtggtgtccaacacgtccaccttcgtgccctatgagatcaaagtccaggccgtcaacagccagggcaagggaccagagccccaggtcactatcggctactctggagaggactacccccaggcaatccctgagctggaaggcattgaaatcctcaactcaagtgccgtgctggtcaagtggcggccggtggacctggcccaggtcaagggccacctccgcggatacaatgtgacgtactggagggagggcagtcagaggaagcacagcaagagacatatccacaaagaccatgtggtggtgcccgccaacaccaccagtgtcatcctcagtggcttgcggccctatagctcctaccacctggaggtgcaggcctttaacgggcgaggatcggggcccgccagcgagttcaccttcagcaccccagagggagtgcctggccaccccgaggcgttgcacctggagtgccagtcgaacaccagcctgctgctgcgctggcagcccccactcagccacaacggcgtgctcaccggctacgtgctctcctaccaccccctggatgaggggggcaaggggcaactgtccttcaaccttcgggaccccgaacttcggacacacaacctgaccgatctcagcccccacctgcggtaccgcttccagcttcaggccaccaccaaagagggccctggtgaagccatcgtacgggaaggaggcactatggccttgtctgggatctcagattttggcaacatctcagccacagcgggtgaaaactacagtgtcgtctcctgggtccccaaggagggccagtgcaacttcaggttccatatcttgttcaaagccttgggagaagagaagggtggggcttccctttcgccacagtatgtcagctacaaccagagctcctacacgcagtgggacctgcagcctgacactgactacgagatccacttgtttaaggagaggatgttccggcaccaaatggctgtgaagaccaatggcacaggccgcgtgaggctccctcctgctggcttcgccactgagggctggttcatcggctttgtgagtgccatcatcctcctgctcctcgtcctgctcatcctctgcttcatcaagcgcagcaagggcggcaaatactcagtgaaggataaggaggacacccaggtggactctgaggcccgaccgatgaaagatgagaccttcggcgagtacaggtccctggagagtgacaacgaggagaaggcctttggcagcagccagccatcgctcaacggggacatcaagcccctgggcagtgacgacagcctggccgattatgggggcagcgtggatgttcagttcaacgaggatggttcgttcattggccagtacagtggcaagaaggagaaggaggcggcagggggcaatgacagctcaggggccacttcccccatcaaccctgccgtggccctagaatagtggagtccaggacaggagatgctgtgcccctggccttgggatccaggcccctccctctccagcaggcccatgggaggctggagttggggcagaggagaacttgctgcctcggatccccttcctaccacccggtccccactttattgccaaaacccagctgcaccccttcctgggcacacgctgctctgccccagcttgggcagatctcccacatgccaggggcctttgggtgctgttttgccagcccatttgggcagagaggctgtggtttgggggagaagaagtaggggtggcccgaaagggtctccgaaatgctgtctttcttgctccctgactgggggcagacatggtggggtctcctcaggaccagggttggcaccttccccctcccccagccactccccagccagcctggctgggactgggaacagaactcggtgtccccaccatctgctgtcttttctttgccatctctgctccaaccgggatgggagccgggcaaactggccgcgggggcaggggaggccatctggagagcccagagtccccccactcccagcatcgcactctggcagcaccgcctcttcccgccgcccagcccaccccatggccggctttcaggagctccatacacacgctgccttcggtacccaccacacaacatccaagtggcctccgtcactacctggctgcggggcgggcacacctcctcccactgcccactggccggcctcttccagccgcccccaccccccaggaccccttagcagccctgccctccctatcgtctgaacagttgtcttcctcagcctcctcccgcccccaccttgggaatgtaaatacaccgtgactttgaaagtttgtacccctgtccttccctttacgccactagtgtgtaggcagatgtctgagtccctaggtggtttctaggattgatagcaattagctttgatgaacccatcccaggaaaaataaaaacagacaaaaaaaaaggaaagattggttctcccagcactgctcagcagccacagcctccctgtatgcctgtgcttggtctactgataagccctctacaaaaaaacaaaagtatatatatatatgtacataatatcagaattataacaggcaaataaaacctgaaaatcaa

SEQ ID NO: 42 - RNA, 5615 nt, Homo sapiens
  source 1..5615: mol_type = mRNA
gcaatttgcaacctccgctgccgtcgccgcagcagccaccaattcgccagcggttcaggtggctcttgcctcgatgtcctagcctaggggcccccgggccggacttggctgggctcccttcaccctctgcggagtcatgagggcgaacgacgctctgcaggtgctgggcttgcttttcagcctggcccggggctccgaggtgggcaactctcaggcagtgtgtcctgggactctgaatggcctgagtgtgaccggcgatgctgagaaccaataccagacactgtacaagctctacgagaggtgtgaggtggtgatggggaaccttgagattgtgctcacgggacacaatgccgacctctccttcctgcagtggattcgagaagtgacaggctatgtcctcgtggccatgaatgaattctctactctaccattgcccaacctccgcgtggtgcgagggacccaggtctacgatgggaagtttgccatcttcgtcatgttgaactataacaccaactccagccacgctctgcgccagctccgcttgactcagctcaccgagattctgtcagggggtgtttatattgagaagaacgataagctttgtcacatggacacaattgactggagggacatcgtgagggaccgagatgctgagatagtggtgaaggacaatggcagaagctgtcccccctgtcatgaggtttgcaaggggcgatgctggggtcctggatcagaagactgccagacattgaccaagaccatctgtgctcctcagtgtaatggtcactgctttgggcccaaccccaaccagtgctgccatgatgagtgtgccgggggctgctcaggccctcaggacacagactgctttgcctgccggcacttcaatgacagtggagcctgtgtacctcgctgtccacagcctcttgtctacaacaagctaactttccagctggaacccaatccccacaccaagtatcagtatggaggagtttgtgtagccagctgtccccataactttgtggtggatcaaacatcctgtgtcagggcctgtcctcctgacaagatggaagtagataaaaatgggctcaagatgtgtgagccttgtgggggactatgtcccaaagcctgtgagggaacaggctctgggagccgcttccagactgtggactcgagcaacattgatggatttgtgaactgcaccaagatcctgggcaacctggactttctgatcaccggcctcaatggagacccctggcacaagatccctgccctggacccagagaagctcaatgtcttccggacagtacgggagatcacaggttacctgaacatccagtcctggccgccccacatgcacaacttcagtgttttttccaatttgacaaccattggaggcagaagcctctacaaccggggcttctcattgttgatcatgaagaacttgaatgtcacatctctgggcttccgatccctgaaggaaattagtgctgggcgtatctatataagtgccaataggcagctctgctaccaccactctttgaactggaccaaggtgcttcgggggcctacggaagagcgactagacatcaagcataatcggccgcgcagagactgcgtggcagagggcaaagtgtgtgacccactgtgctcctctgggggatgctggggcccaggccctggtcagtgcttgtcctgtcgaaattatagccgaggaggtgtctgtgtgacccactgcaactttctgaatggggagcctcgagaatttgcccatgaggccgaatgcttctcctgccacccggaatgccaacccatggagggcactgccacatgcaatggctcgggctctgatacttgtgctcaatgtgcccattttcgagatgggccccactgtgtgagcagctgcccccatggagtcctaggtgccaagggcccaatctacaagtacccagatgttcagaatgaatgtcggccctgccatgagaactgcacccaggggtgtaaaggaccagagcttcaagactgtttaggacaaacactggtgctgatcggcaaaacccatctgacaatggctttgacagtgatagcaggattggtagtgattttcatgatgctgggcggcacttttctctactggcgtgggcgccggattcagaataaaagggctatgaggcgatacttggaacggggtgagagcatagagcctctggaccccagtgagaaggctaacaaagtcttggccagaatcttcaaagagacagagctaaggaagcttaaagtgcttggctcgggtgtctttggaactgtgcacaaaggagtgtggatccctgagggtgaatcaatcaagattccagtctgcattaaagtcattgaggacaagagtggacggcagagttttcaagctgtgacagatcatatgctggccattggcagcctggaccatgcccacattgtaaggctgctgggactatgcccagggtcatctctgcagcttgtcactcaatatttgcctctgggttctctgctggatcatgtgagacaacaccggggggcactggggccacagctgctgctcaactggggagtacaaattgccaagggaatgtactaccttgaggaacatggtatggtgcatagaaacctggctgcccgaaacgtgctactcaagtcacccagtcaggttcaggtggcagattttggtgtggctgacctgctgcctcctgatgataagcagctgctatacagtgaggccaagactccaattaagtggatggcccttgagagtatccactttgggaaatacacacaccagagtgatgtctggagctatggtgtgacagtttgggagttgatgaccttcggggcagagccctatgcagggctacgattggctgaagtaccagacctgctagagaagggggagcggttggcacagccccagatctgcacaattgatgtctacatggtgatggtcaagtgttggatgattgatgagaacattcgcccaacctttaaagaactagccaatgagttcaccaggatggcccgagacccaccacggtatctggtcataaagagagagagtgggcctggaatagcccctgggccagagccccatggtctgacaaacaagaagctagaggaagtagagctggagccagaactagacctagacctagacttggaagcagaggaggacaacctggcaaccaccacactgggctccgccctcagcctaccagttggaacacttaatcggccacgtgggagccagagccttttaagtccatcatctggatacatgcccatgaaccagggtaatcttggggagtcttgccaggagtctgcagtttctgggagcagtgaacggtgcccccgtccagtctctctacacccaatgccacggggatgcctggcatcagagtcatcagaggggcatgtaacaggctctgaggctgagctccaggagaaagtgtcaatgtgtaggagccggagcaggagccggagcccacggccacgcggagatagcgcctaccattcccagcgccacagtctgctgactcctgttaccccactctccccacccgggttagaggaagaggatgtcaacggttatgtcatgccagatacacacctcaaaggtactccctcctcccgggaaggcaccctttcttcagtgggtctcagttctgtcctgggtactgaagaagaagatgaagatgaggagtatgaatacatgaaccggaggagaaggcacagtccacctcatccccctaggccaagttcccttgaggagctgggttatgagtacatggatgtggggtcagacctcagtgcctctctgggcagcacacagagttgcccactccaccctgtacccatcatgcccactgcaggcacaactccagatgaagactatgaatatatgaatcggcaacgagatggaggtggtcctgggggtgattatgcagccatgggggcctgcccagcatctgagcaagggtatgaagagatgagagcttttcaggggcctggacatcaggccccccatgtccattatgcccgcctaaaaactctacgtagcttagaggctacagactctgcctttgataaccctgattactggcatagcaggcttttccccaaggctaatgcccagagaacgtaactcctgctccctgtggcactcagggagcatttaatggcagctagtgcctttagagggtaccgtcttctccctattccctctctctcccaggtcccagccccttttccccagtcccagacaattccattcaatctttggaggcttttaaacattttgacacaaaattcttatggtatgtagccagctgtgcactttcttctctttcccaaccccaggaaaggttttccttattttgtgtgctttcccagtcccattcctcagcttcttcacaggcactcctggagatatgaaggattactctccatatcccttcctctcaggctcttgactacttggaactaggctcttatgtgtgcctttgtttcccatcagactgtcaagaagaggaaagggaggaaacctagcagaggaaagtgtaattttggtttatgactcttaaccccctagaaagacagaagcttaaaatctgtgaagaaagaggttaggagtagatattgattactatcataattcagcacttaactatgagccaggcatcatactaaacttcacctacattatctcacttagtcctttatcatccttaaaacaattctgtgacatacatattatctcattttacacaaagggaagtcgggcatggtggctcatgcctgtaatctcagcactttgggaggctgaggcagaaggattacctgaggcaaggagtttgagaccagcttagccaacatagtaagacccccatctctttaaaaaaaaaaaaaaaaaaaaaaaaaaaactttagaactgggtgcagtggctcatgcctgtaatcccagccagcactttgggaggctgagatgggaagatcacttgagcccagaattagagataagcctatggaaacatagcaagacactgtctctacaggggaaaaaaaaaaaagaaactgagccttaaagagatgaaataaattaagcagtagatccaggatgcaaaatcctcccaattcctgtgcatgtgctcttattgtaaggtgccaagaaaaactgatttaagttacagcccttgtttaaggggcactgtttcttgtttttgcactgaatcaagtctaaccccaacagccacatcctcctatacctagacatctcatctcaggaagtggtggtgggggtagtcagaaggaaaaataactggacatctttgtgtaaaccataatccacatgtgccgtaaatgatcttcactccttatccgagggcaaattcacaaggatccccaagatccacttttagaagccattctcatccagcagtgagaagcttccaggtaggacagaaaaaagatccagcttcagctgcacacctctgtccccttggatggggaactaagggaaaacgtctgttgtatcactgaagttttttgttttgtttttatacgtgtctgaataaaaatgccaaagttttttttca

SEQ ID NO: 43 - RNA, 1894 nt, Homo sapiens
  source 1..1894: mol_type = mRNA
acagttgctgctaactgccctggtgtgtgtgagggagagagagggagggagggagagagagcgcgctagcgcgagagagcgagtgagcaagcgagcagaaaagaggtggagagggggggaataagaaagagagagaaggaaaggagagaaggcaggaagaaggcaagggacgagacaaccatgctgtgctgtatgagaagaaccaaacagaattaaaagggaacctggtctctgggttgttttcaacatctcaagtgtgaattttccctgtcaaaatcttcacaaggaaaatgagtcacagcatcacctgggtgacgaggtcataacacctcagcccttgcttaaaaaattttatttctacttttctattgtaaagagatctcaaaacaggaagataaaattggactgacagctctacagcctagtcttttagacagtgaactaggccagcattggcagacactggcgatgacaaagtcctgctctgaattatgccaccccgcactccactttttaccttgcctgggaggcttgaggaaaaatcttcagagagcagttcgacctagtccttattcacttggcttcttgactttctggatttcaagggttgaaaaaaatgatgacgaccaaaagattgaacaagatggtatcaaaccagaagataaagctcataaggccgcaaccaaaattcaggctagcttccgtggacacataacaaggaaaaagctcaaaggagagaagaaggatgatgtccaagctgctgaggctgaagctaataagaaggatgaagcccctgttgccgatggggtggagaagaagggagaaggcaccactactgccgaagcagccccagccactggctccaagcctgatgagcccggcaaagcaggagaaactccttccgaggagaagaagggggagggtgatgctgccacagagcaggcagccccccaggctcctgcatcctcagaggagaaggccggctcagctgagacagaaagtgccactaaagcttccactgataactcgccgtcctccaaggctgaagatgccccagccaaggaggagcctaaacaagccgatgtgcctgctgctgtcactgctgctgctgccaccacccctgccgcagaggatgctgctgccaaggcaacagcccagcctccaacggagactggggagagcagccaagctgaagagaacatagaagctgtagatgaaaccaaacctaaggaaagtgcccggcaggacgagggtaaagaagaggaacctgaggctgaccaagaacatgcctgaactctaagaaatggctttccacatccccaccctcccctctcctgagcctgtctctccctaccctcttctcagctccactctgaagtcccttcctgtcctgctcacgtctgtgagtctgtcctttcccacccactagccctctttctctctgtgtggcaaacatttaaaaaaaaaaaaaaaaagcaggaaagatcccaagtcaaacagtgtggcttaaacattttttgtttcttggtgttgttatggcaagtttttggtaatgatgattcaatcattttgggaaattcttgcactgtatccaagttatttgatctggtgcgtgtggccctgtgggagtccactttcctctctctctctctctctgttccaagtgtgtgtgcaatgttccgttcatctgaggagtccaaaatatcgagtgaattcaaaatcatttttgttttcctccttttcaatgtgatggaatgaacaaaaaggaaaaaattcaaaaaacccagtttgttttaaaaataaataaataaagcaaatgtgccaattagcgtaaacttgcggctctaaggctcctttttcaacccgaatattaataaatcatgagagtaatcaa

SEQ ID NO: 44 - RNA, 4171 nt, Homo sapiens
  source 1..4171: mol_type = mRNA
agcctgcgctcctggcgccctcatgtcttcacgggactccccgcgccggttgacgtggtgtctcgttcggatctccaggcaagacctcagctccggcagcagcatcagacaaaccagtctcgtgctcccaggcagtgcgcccagaggaggcgcagagcgcggcagctgccgctgagccgcccgcagcgccccgggcccgcgcagccccagccaagagcgcgacgcgcgcagccctgtcagccccccacccaaagcagcggcggctgctcggacctcggcttctgggggtgcgggggcccggcgggagagttgccggcagccctcgccctgttggcggcggcggcggcgggagtcttggccgccgcctccagcgcgcccttgctgccccgcgcgaccccaggattgcgaactcttgcccctgacctgttgggcggggctccgcgctccagccatcagcccggatgggtctcctggctgggacttggggcacctggagttaatgtccaacctagggtctgcggagacccgatccgaggtgccgccgccggacgggactttaagatgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctgactccggctccagagactgctgtgtattgcattcctgctacagtgcaaagaaagggaccaaggttcccaggaaatgtttgcccagaatggaagatgaggaccaaggaggcggaggaggaggaagaagaagaggaggaggaggaggaggaggaggaggaggaggaaggcagccatcatgggagcctggtagagggagatccagctacagacaactggacaggagagagagaaaacagccctctggattctccaggatggcagccgatgtcactagaagctcagggctgatgttcctggttggctattgccaccatttcagctgatacagtccaccatcactgattaccggcgcggttgcggtggatgcacttgaaccaaaccagtgtatctcctgtgatttgttttcatgtgtccgaagacaccagggaaacagagatcctggtgttgttccttgttattacgttttactgctgaaagtaagaggtttatttttctgtcactcagtggagacataccctggaaaggagaggggaaaaaaaaagcaaagatacaagagataatcacctttgcatttgaaagttgaggcccgaggtttactacaaccagcatttttgccaacggttggtgattgatttccatcacggtgtgtggggtgggaagaagttggctaggaaccaaaaaggctgtgctcatgattaaacacaaacctgaaggtatttcctttatgtccttggaaacaggaaacgagttgtggttttcgccagcattcttgtaggagagaatcggggaaggccccgaactgcccccgggcagggagagcccctcaggcctgttggtttacagagagacagatgttacataaccagctccgttgatgcgtggtcaccagtgaccagagaagctactcgatgcaatgcatctgtttcagatacagaaatatagagaagatatttattgaaatttaagttattgttatttattaccgttcactaatgaatttctcttttttcccttatttattaaagtttcttttcaaaggtgccaaagtatatgtgctcgcaaaatgcaaagaaaggtgacaaaaggaaatttgaattgggaacaagggtccatgcttttcaaagtattaaaaagttttttgccaggcaaaaatcacttactttacctttttaagaaaatttgtcattaattttccccagatttcagcatttttcccaatttttatttgtggagcatctcaggcaagccccctttcctggagcagcgtgcagagaccactggcacttgactttatttcttccttgctccattgctgaacagaaatgtcgtgggctccacttcctgttgtctttaagctcttagtcccctccacgtatacctatctgtactatgcataaccatatgtagaaaaggttcagttccttttagtaggtagtcctggatttaatgctgacctaaaagtaatgtcgacaatgctgtcaggtagctgccgttctaccgactccctccatccctgcccacccactgccctcccgagaatatgctggctgcccagtgcagcccgggagacacaggggccttccagaggtagggtctaccaggtcctgtacaacccctgggctgtcaccgggggtcaacagctgctgctcctatatacccaaacacctgacagctccctggggagcagatggctgagaagggtgctgaggaagccatattgggaccagccacagccacacacatggagcctcatacttaggagcgtgctgcctttaaatgaaggtggtcggggccagtgcagcggctcacacccataatcccaacactttggaaagccaaggtgggaggatctcttgaacccaggagtttgagaccagcttgggcaacatagggagaccctgtctctacagaaactttaaaaattaggcaggcatgatggtgcacacctgtggtcccagctactcaagaggctgaaggaggatcacttgagtccagaaggtcgaggctgcagtgagctgtgatcatgccactgcactccagcctaagtgacagtgcggtaccctgtctcaaaaaaaaaaaaaaaaaaaaaaagaggttggagcaggaggaagcataggggcgggaacagccacctcctccatgccctagattgtgaatttatcgggcagccaacacatgtatgacacactaggccctgtattacagcttgttacgcatttcataaaagggattttcattaaggagataatctattactacctaccttagtggctactagtataaaactatgacagatttagcaattaaatgaaatactggcctccatcaaataatcatagtaacaagaagcagcagttaccagacatctgatccccttcccccaaaatacccaaattcttcatggttctgcccttctctgtcctttctgctccccttgctcgcctgggaaatggaggaaaggccttccctctcacactgtcttgggatcttgctgagaattcagactgctcgaaacagtgacaaaccccagccatccagtcattcgtggagcacaatttggatgtggccccaggggcatctgtcccattcagagaaccttggcagtgcgatggccactgttcccaggcttcaacctcagtgaccccccccaacaactccccatggagagtccctgcccaaaaaagctgtaggatccaaggggtgtcaatagctcgttcccggcatcacctacacaccacaagcaggttttaatggaagcaagttgctccaccaaatccacaaaagggtaaagtttgtgatttttctttatcattgcgatcaccatctgataccgtaaggagtgcacttgtttggaagttctgacttctctgatctgtcttggtcgtttgtgttataaaaccaaagttctctacagactttatttttgtacaatatcattttgtaactttttacaaataaaaactcatttctattgc

SEQ ID NO: 45 - RNA, 2402 nt, Homo sapiens
  source 1..2402: mol_type = mRNA
gagctgggctggcggagcagaggtgcagaagcaactgagtccaagttgtctggcggcttcaggtggacccagaagacgtccccaactcagggagattcagcgatcactcactcgctgtacagaatgatattcctcacggcactgcctctgttctggattatgatttcagcctcccgagggggtcactggggtgcctggatgccctcgtccatctcggccttcgaaggcacgtgcgtctccatcccctgccgctttgacttcccggatgagctgcggcccgctgtggtgcatggtgtctggtacttcaatagcccctaccccaagaactaccccccggtggtcttcaagtcgcgcacccaagtagtccacgagagcttccagggccgcagccgcctcctgggggacctgggcctgcgaaactgcaccctcctgctcagcaacgtcagccccgagctgggcgggaagtactacttccgtggggacctgggcggctacaaccagtacaccttctcagagcacagcgtcctggatatcgtcaacacccccaacatcgtggtgcccccagaggtggtggcaggcacggaggtggaggtcagctgcatggtgccggacaactgcccagagctgcgccctgagctgagctggctgggccacgaggggctgggggagcccgctgtgctgggccggctgcgggaggacgagggcacctgggtgcaggtgtcactgctgcacttcgtgcccacgagggaggccaacggccacaggctgggctgccaggcctccttccccaacaccaccctgcagttcgagggctacgccagcatggacgtcaagtaccccccggtgattgtggagatgaactcctcggtggaggccatcgagggctcccacgtgagcctgctctgtggggctgacagcaaccccccgccgctgctgacctggatgcgggacgggacagtcctccgggaggcggtggccgagagcctgctcctggagctggaggaggtgacccccgccgaagacggcgtctatgcctgcctggccgagaatgcctatggccaggacaaccgcaccgtggggctcagtgtcatgtatgcaccctggaagccaacagtgaacgggacaatggtggccgtagagggggagacggtctctatcttgtgctccacacagagcaacccggaccctattctcaccatcttcaaggagaagcagatcctgtccacggtcatctacgagagcgagctgcagctggagctgccggccgtgtcacccgaggatgatggagagtactggtgtgtggctgagaaccagtatggccagagggccaccgccttcaacctgtctgtggagttcgcccctgtgctcctcctggagtcccactgcgcggcagcccgagacacggtgcagtgcctgtgcgtggtgaagtccaacccggagccgtccgtggcctttgagctgccatcgcgcaatgtgaccgtgaacgagagcgagcgggagttcgtgtactcggagcgcagcggcctcgtgctcaccagcatcctcacgctgcgggggcaggcccaggccccgccccgcgtcatctgcaccgcgaggaacctctatggcgccaagagcctggagctgcccttccagggagcccatcgactgatgtgggccaagatcgggcctgtgggcgccgtggtcgcctttgccatcctgattgccatcgtctgctacattacccagacacgcaggaaaaagaacgtgacagagagccccagcttctcggcaggggacaaccctcccgtcctgttcagcagcgacttccgcatctctggggcaccagagaagtacgagtccaaagaggtttctaccctggaatctcactgagtgccccaggagagcgagaggcgcctgggatctgagaggaggctgctgggccttcggggtgagcccccagagctggacctgagctattctcactcggacctggggaaacggcccaccaaggacagctacacgctgacggaggagctagctgagtatgctgaaatccgggtcaagtgaaggagctgggggcagcctgcgtggctgacccccctcaggaccctcgctggcccccactggctgtgggctcccttcctcccaaaagtatcgggggctggggcaggaggggagtgaggcaggtgacagtgaggtcctgggggcctgacctccccctccttcccagctgcccctccctgccagcacccccacgccctcattacggctcctctctaacctcctttaccctcatctgtctggaggggagctctgtctgtccgtgttatttattgctacttcctgcctggtctcctgcccccacacctggccctggggcctgtacaaaagggacatgaaataaatgccccaaagccaaa

SEQ ID NO: 46 - RNA, 1951 nt, Homo sapiens
  source 1..1951: mol_type = mRNA
agttcctggtcccccactttctcaaccccacagatgctccgggcccctgcccctgccccagctatggctcctggggctccctcatccagccccagccctatcctggctgtgctgctcttctcttctttggtgctgtccccggcccaggccatcgtggtttacaccgacagggaggtccatggtgctgtgggctcccgggtgaccctgcactgctccttctggtccagtgagtgggtctcagatgacatctccttcacctggcgctaccagcccgaagggggcagagatgccatttcgatcttccactatgccaagggacaaccctacattgacgaggtggggaccttcaaagagcgcatccagtgggtaggggaccctcgctggaaggatggctccattgtcatacacaacctagactacagtgacaatggcacgttcacttgtgacgtcaaaaaccctccagacatagtgggcaagacctctcaggtcacgctgtatgtctttgaaaaagtgccaactaggtacggggtcgttctgggagctgtgatcgggggtgtcctcggggtggtgctgttgctgctgctgcttttctacgtggttcggtactgctggctacgcaggcaggcggccctgcagaggaggctcagtgctatggagaaggggaaattgcacaagccaggaaaggacgcgtcgaagcgcgggcggcagacgccagtgctgtatgcaatgctggaccacagcagaagcaccaaagctgtcagtgagaagaaggccaaggggctgggggagtctcgcaaggataagaaatagcggttagcgggccgggcgggggatcgggggttaggggtggagtccgccaaaggcccaaaggtgatggtcatcgagatggagctacgaaaggatgagcagagcccggagctccggcctgctgtcaagtcccccagcagaaccagcctcaaaaacgccctcaagaacatgatgggcctgaactcggacaagtgatcgccacccccccaccccaggccctgccagagcagggggacctaggctcctcttacccccgtctaggtgctttccctctttgctcccccgccctgccctgccctcacctccctttgagatgtaagtttcattccagaattcattccccaggcaattgtattctcccccaccttcacccctggctttctgggagcccaggagctaatcctacccctcacctgccccgggggctgtgtgtttggtgcctgtccacctgagcactgagaagaaagggactttgataccctctgcctcaagtccaggccacctggcattcccatctcctgcatcccccagcctgtccccctggctgtttcctctcccgtccctccctcccctctaccaggtggcccagctccatactctgtccccccagctaatacccagagcacccagatcagactctccttcagggtttatttaggttattattttttattttttaatccattctttgtttgtttacctgtgctcatcctctgcccttacacccatgactgaggaccaatgacgtcatgtggcttttgcaattcacgccccccttaagtccttaatgaagagccagcccaagtagaggggcccctgatcctcacacttcagtatagcattggttccccctgaccactttggagcactgttctgggactccaggtcttgaggagagagacagagagagagaatggatcctcataggtcagggagtgggggagggggcaaatgagccttaagaaatggtttttaaacaaccaaacaaaaagcaggaaaaacaaatgggaaatgggggggcgggggggaggaagaggctgcactgcagccacaggggattcttaggatttttctacattctgtatatttcttctcaaacctccaaatgtccttaaatgtttaataaacactgacatttccagaa

SEQ ID NO: 47 - RNA, 3408 nt, Homo sapiens
  source 1..3408: mol_type = mRNA
agagcgagccgagccgcggccagctccggcgggcagggggggcgctggagcgcagcgcagcgcagccccatcagtccgcaaagcggaccgagctggaagtcgagcgctgccgcgggaggcgggcgatgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttcaagaggtggaacagctgcaagcagaacaagcaaggagccaacagccggccagtgaaccagacgcccccaccagagggagaaaaactccacagcgacagtggcatctccgtggacagccagagcctgcatgaccagcagccccacacgcagacagcctcgggccaggccctcaagggtgacggaggcctctacagcagcctgcccccagccaagcgggaggaggtggagaagcttctcaacggctctgcgggggacacctggcggcacctggcgggcgagctgggctaccagcccgagcacatagactcctttacccatgaggcctgccccgttcgcgccctgcttgcaagctgggccacccaggacagcgccacactggacgccctcctggccgccctgcgccgcatccagcgagccgacctcgtggagagtctgtgcagtgagtccactgccacatccccggtgtgagcccaaccggggagcccccgccccgccccacattccgacaaccgatgctccagccaacccctgtggagcccgcacccccaccctttggggggggcccgcctggcagaactgagctcctctgggcaggacctcagagtccaggccccaaaaccacagccctgtcagtgcagcccgtgtggccccttcacttctgaccacacttcctgtccagagagagaagtgcccctgctgcctccccaaccctgcccctgccccgtcaccatctcaggccacctgcccccttctcccacactgctaggtgggccagcccctcccaccacagcaggtgtcatatatggggggccaacaccagggatggtactagggggaagtgacaaggccccagagactcagagggaggaatcgaggaaccagagccatggactctacactgtgaacttggggaacaagggtggcatcccagtggcctcaaccctccctcagcccctcttgccccccaccccagcctaagatgaagaggatcggaggcttgtcagagctgggaggggttttcgaagctcagcccacccccctcattttggatataggtcagtgaggcccagggagaggccatgattcgcccaaagccagacagcaacggggaggccaagtgcaggctggcaccgccttctctaaatgaggggcctcaggtttgcctgagggcgaggggagggtggcaggtgaccttctgggaaatggcttgaagccaagtcagctttgccttccacgctgtctccagacccccaccccttccccactgcctgcccacccgtggagatgggatgcttgcctagggcctggtccatgatggagtcaggtttggggttcgtggaaagggtgctgcttccctctgcctgtccctctcaggcatgcctgtgtgacatcagtggcatggctccagtctgctgccctccatcccgacatggacccggagctaacactggcccctagaatcagcctaggggtcagggaccaaggacccctcaccttgcaacacacagacacacgcacacacacacacaggaggagaaatctcacttttctccatgagttttttctcttgggctgagactggatactgcccggggcagctgccagagaagcatcggagggaattgaggtctgctcggccgtcttcactcgcccccgggtttggcgggccaaggactgccgaccgaggctggagctggcgtctgtcttcaagggcttacacgtggaggaatgctcccccatcctccccttccctgcaaacatggggttggctgggcccagaaggttgtgatgaagaaaagtgggccagtgtgggaatgcggcaagaaggaattgacttcgactgtgacctgtggggatttctcccagctctagacaaccctgcaaaggactgttttttcctgagcttggccagaagggggccatgaggcctcagtggactttccaccccctccctggcctgttctgttttgcctgaagttggagtgagtgtggctcccctctatttagcatgacaagccccaggcaggctgtgcgctgacaaccaccgctccccagcccagggttcccccagccctgtggaagggactaggagcactgtagtaaatggcaattctttgacctcaacctgtgatgaggggaggaaactcacctgctggcccctcacctgggcacctggggagtgggacagagtctgggtgtatttattttcctccccagcaggtggggagggggtttgggggcttgcaagtatgttttagcatgtgtttggttctggggcccctttttactccccttgagctgagatggaacccttttggcccccgagctgggggccatgagctccagacccccagcaaccctcctatcacctcccctccttgcctcctgtgtaatcatttcttgggccctcctgaaacttacacacaaaacgttaagtgatgaacattaaatagcaaagaaagaaaaa

SEQ ID NO: 48 - RNA, 1497 nt, Homo sapiens
  source 1..1497: mol_type = mRNA
gacgcctcctgcagcgcctggagccacacagggatccggagcctgggggaaaagcggcgcgggagccggcacccaccgctggaggggcggcgacggcggccgtagcgacctcgggaggcaagcggagccgccatggccgagttcccgtcgaaagttagcacgcggaccagcagtcctgcgcagggcgccgaagcctcggtgtcggcgctgcgcccggacctgggcttcgtgcgctcccgcctcggggcgctcatgctgctgcagctggtgctggggctgctggtgtgggcgctgattgcggacaccccgtaccacctgtatccggcctatggctgggtgatgttcgtcgctgtcttcctctggctggtgacaatcgtcctcttcaacctctacctgtttcagctgcacatgaagttgtacatggttccctggccactggtgttaatgatctttaacatcagcgccaccgttctctacatcaccgccttcatcgcctgctctgcggcagttgacctgacatccctgaggggcacccggccttataaccagcgcgcggctgcctcgttctttgcgtgtttggtgatgatcgcctatggagtgagtgccttcttcagctaccaggcctggcgaggagtaggcagcaatgcggccaccagtcagatggctggcggctatgcctaaaccacctgtgccacggccccctctggggctgaagccgccgctgggtcacagagcagggtcaccctgcaagcctgaagctggggagccctgcgtggagtcagcccaacagggactgcatttgctcctctctgcccgtcagacataagctctcacagcgctaaggaagcaggcccaggctggcaggcatctcggcttgcaggaggccaactgctgagacctcttctccatcccccttattcagtggaagatgacgggggatctgaggctgtgtctctgccttgtctttagaggacttcagcgtccaagactggggcccacccttctcaccagcactaaatgcactaacaaggactccagacctgcagccccagacccgccgtagtataagcctaacaagcaacacgtagcaccttagtctttgttccaggagagctgagcaagctggtgaaaccactctccttcctttaaacaccgtttcaaccaacctctccctggagccaacctgtaaaaagtgggttgattgctgacagcatggtcttccctccctgcatttcagacataccagttactgaaagcaaatcagttttaagtgatttctcagtgctgaaaagcctgtccaggtttccttccctttcccaagcctctctctgtaatactccctttgggcgaagctaacatcggtgcctccccgaccttgctgactaggcacatgggacgcaaaggagggagggaagcaaggccttgcctggcgagttgtcatgtggttggtggtgactgttttattttttttaataaaaataaagatgagagaaatta

SEQ ID NO: 49 - RNA, 6319 nt, Homo sapiens
  source 1..6319: mol_type = mRNA
gagacgtgctcctggcaccgccagctgctacttggccctcgccggtggcccaccaggacaatgcccccccgcagccatctcatgcccatcgccactgccctggggcagctgaactgagcgtatgtgccacgccgcccaggagacccctctgctgcaccacttcatgcacccccgctgccttgccccagtgcttcctcgccctggagctgggcgccagcatggagctcacccctgcctcttcgctgacttgctccttgctcagcccgcggctgcctggctctttcccccagctgcggagggttcctccttgcagccggccctggctgcccaaggcccccgtgatggctcgctggcctcccttcggcctctgcctcctcctgctgctgctgtccccaccgccactgcccttgacaggggcccatcgcttctccgcacctaataccactctcaaccacttggcactggcacctggccgaggcacactctatgtcggcgcagtgaaccgcctcttccagctcagccccgagctgcagctcgaggccgtggctgtcactggccctgtaatcgacagccctgactgcgtgcccttccgtgacccagccgagtgcccacaggcccagctcactgacaatgccaaccagctgctgctggtgagcagccgcgcccaggagctggtggcctgcgggcaggtgcggcagggcgtgtgtgagacacggcgccttggggatgtggccgaggtgctgtaccaggctgaggaccctggtgacgggcagtttgtggctgccaataccccgggagtggcaacggtggggctggtggtgcccttgcccggccgggacctcctgcttgtggccagaggcctggcgggcaagctgtcggcaggggtgccacccctggccatccgccagctggccgggtctcagcccttctccagcgagggcctgggccgcctggtggtgggcgacttctccgactacaacaacagctacgtcggggcctttgccgacgcccgctccgcctacttcgtgttccgccgccgcggggcccgggcccaggctgagtaccgctcctacgtggcccgcgtctgcctgggggacaccaacctgtactcctacgtggaggtccccctcgcctgccagggccagggcctcatccaggccgccttccttgccccgggcaccttgctaggggtgtttgccgcgggcccaaggggcacccaggcggcgctctgtgccttccccatggtggagctgggtgccagcatggagcaggcccggagactctgctacacggcgggcggccggggccccagcggcgcagaggaagccaccgtggagtacggcgtcacgtcgcgctgcgtcaccctgccccttgattcccccgagtcgtacccctgtggcgacgagcacacccccagccccattgctggccgccagcccctggaggtccagcctctgctgaagctcgggcagccggtcagcgccgtggcagctctccaggcagatgggcacatgatagccttcctgggggacacccagggccagctgtacaaggtctttctccacggctcccagggccaggtttaccactcccagcaagtggggcctccaggctcagccatcagcccagacctgctgctggacagcagtggcagtcacctctatgtcctgactgcccaccaggtggaccggatacctgtggcagcctgcccccagttccctgactgtgccagctgcctccaggcccaggacccgctgtgtggctggtgtgtcctccagggcaggtgtacccggaagggccagtgcgggcgggcaggccagctgaaccagtggctgtggagttatgaggaggacagccactgcctgcacatccagagcctgctgccgggccaccacccccgccaggagcagggccaggtcactttgtctgtcccccggctgcccatcctggatgcagatgaatacttccattgtgcgttcggggactatgacagcttggctcatgtggaagggccccacgtggcctgtgtcacccctccccaagaccaggtgccacttaaccctccaggcacagaccacgtcactgtgcccctggccctgatgttcgaggacgtgactgtggctgccaccaacttctccttttatgactgcagtgccgtccaggccttggaggcggctgccccgtgtcgcgcttgcgtgggcagcatctggcggtgtcactggtgcccgcagagtagccactgcgtgtacggagagcactgcccagagggcgagaggaccatctacagcgcccaggaggtggacatccaggtgcgtggcccaggggcttgcccacaggtcgaaggcctggcaggtccccacctggtgcctgtgggctgggagagccatttggccctacgcgtgcggaaccttcaacatttccgaggcctgcctgcctccttccactgctggctggagctgcctggagaacttcggggactgccggccaccctggaggagacagcaggggattcaggcctcatccactgccaggcccaccagttttatccctccatgtcccagcgggagctcccagtgcccatctacgtcacccagggtgaagcccagaggctggacaacacccatgctctttatgtgatcctgtacgactgcgccatgggccacccggactgcagccactgccaagcggccaacaggagcctgggctgcctgtggtgtgctgacggccagcctgcctgtcgctatgggcccttgtgcccgccgggggctgtggagctgctgtgtcctgcgcccagcattgatgcagtcgagcccctgaccggtccccctgagggaggcttggccctcaccatcctgggctccaacctgggccgggccttcgccgatgtgcagtacgccgtgagcgtggccagccggccctgcaaccctgagccctctctctaccgcacgtcggcccggattgtgtgtgtgacatctcctgcccccaatggcaccactgggcccgtccgggtggccattaagagccagccaccaggcatctcaagccagcacttcacctaccaggaccctgtcctgctgagcctgagtcctcgctggggcccccaggcagggggcacccagctcaccatccgaggtcagcacctccagacaggtggcaacaccagtgccttcgtgggtggccaaccctgtcccatcctggagccagtgtgtccggaggccatcgtgtgccgtaccaggccccaggctgccccaggagaagcagcggtccttgtggtctttggccatgcccagcgcacactgctcgccagccccttccgctacaccgccaacccccagcttgtagcggcggagcccagtgccagcttccgggggggtgggcgactgatccgtgtcaggggcaccggcctagacgtggtgcagcggcccctactgtctgtgtggctggaggctgacgcagaggtgcaggcttccagggcccagccccaggacccacagccaaggaggagctgtggagcccctgctgcggacccccaggcttgtatccagctcggtggggggctgctgcagtgctccaccgtctgctccgtcaactcgtccagcctcctcctgtgccggagccctgctgtaccagacagagcccacccgcagcgggtcttcttcaccctagacaacgtgcaagtggacttcgccagtgccagtgggggccagggcttcctgtaccagcccaacccccgcctggcacccctcagccgcgaggggcctgcccgcccctaccgcctcaagccaggccatgtcctggatgtggagggcgagggcctcaacctgggcatcagcaaggaggaggtgcgcgtgcacatcggccgcggcgagtgcctggtgaagacgctcacgcgcacccacctgtactgcgagccgcctgcgcacgccccgcagcctgccaatggctccggcctgccacagttcgtggtgcagatgggcaatgtgcagctggccctgggccctgtgcagtacgaggctgaacccccgctgtctgcctttcccgtggaggcccaggcaggcgtgggcatgggtgctgcagtgctgattgccgccgtgctcctcctcaccctcatgtacaggcacaagagcaagcaggccctgcgggactaccagaaggtgctagtgcagctggagagcctggagaccggcgtgggagaccagtgccgcaaggagttcacagacctcatgacggagatgaccgacctcagcagcgacctggagggcagcgggatccccttcctggactaccgcacctacgccgagcgcgccttcttccctggccatggcggttgcccgctgcagcccaagcctgaggggccaggggaggacggccactgtgccactgtgcgccagggcctcacgcagctctccaacctgctcaacagcaagctcttcctcctcacgctcatccacaccctggaggagcagcccagcttttcccagagggatcgctgccatgtggcttcgctgctgtcgctagcgctacacggcaagctggagtacctgacggacatcatgaggaccctgctgggtgacctggcggcccattacgtgcacaggaaccccaagctcatgctacgcaggacagagaccatggtggagaaactgctcaccaactggctgtccatctgcctgtacgccttcctgagggaggtggctggtgaaccactgtacatgctcttccgggccatccagtaccaggtggacaaaggccccgtggacgccgtgacaggcaaggccaaacggaccctgaatgatagccgcttgctgcgggaggacgtggagttccagcccctgacgctgatggtgctggtggggcccggggctggcggggccgcaggcagcagcgagatgcagcgcgtgccagcccgggtgctcgacacggacaccatcacccaggtcaaggagaaggtgttggaccaagtctacaagggcacccccttctcccagaggccctcagtgcatgccctagaccttgagtggcgctcaggcctggctggtcacctgaccctatcggacgaagacttgacctccgtgacccaaaaccactggaagagactcaacaccttgcaacactacaaggtcccagatggagcaacagtggggctcgtccctcagctgcaccgtggcagcaccatctcccagagcctggcccagaggtgccccttgggagagaacatacccacgctggaggatggcgaggaggggggggtgtgcctctggcacctggtgaaagccaccgaggagccagaaggggccaaggtgcggtgcagcagcctgcgggagcgcgagccagcaagggccaaggccattccggaaatctacctcacccgtctgctgtccatgaagggcacgctgcagaagtttgtggacgacaccttccaggccattctcagcgtgaaccggcccatccccatcgccgtcaagtacctgtttgaccttctggatgagctagcagagaagcacggcatcgaggacccagggaccctgcacatctggaagaccaacagtctgctgctgcggttctgggtgaatgccttgaagaacccacagctcatctttgatgtacgggtgtcggacaatgtggacgccatccttgctgtcatcgcccagaccttcattgactcctgtaccacctcggagcataaagtgggccgggattccccagtgaacaaactgctctacgcccgggagatcccacgctacaagcagatggtggagaggtactatgcggacattcgccagagctctccggcgagctaccaggagatgaactctgctttggctgagctctccgggaactacacttctgctccccactgtctggaggctctgcaagaactctacaaccacatccacaggtactatgatcagattatcagtgccctggaggaggaccctgtgggccagaagctgcagctggcctgccgcctgcagcaggtcgccgccctggtggaaaacaaagtgactgacctgtgagctctggctcagacagcagcaagccggatccaccaacaccgcagcgccttatgaccccggaaccgagccagccactgaggggagctggcagagcctgggggcacagggtgcaaagccaggcactgtgcccagcagtgggctccctgcctgccacctcccctgccagcccacccaccttccccccacctgagattgtttctaatttataaggatccccctccttccccctctccccattgtatttatttgcctgctggaaaatcacatccggaaataaaatagaaatatgtctttttatttta

SEQ ID NO: 50 - RNA, 4885 nt, Homo sapiens
  source 1..4885: mol_type = mRNA
agagagctggagagagcagggagaggggggagcgccgagctagtcagagagtgagcgagagcgagaaggagggagaggaggagaaagagagcgagggcgggcgggaggcggcggcggcggcagcagcagcagtaatagcaggagcagcaacagaaggcgtcggagcgggcgtcggagctgcccgctgtgggagagagaggagacagaaagagcgagcgaggagagggagcccgaggcgaaaaagtaactgtcaaatgcgcggctcctttaaccggagcgctcagtccggctccgagagtcatggcgaccgcagcgtctaaccactacagcctgctcacctccagcgcctccatcgtgcacgccgagccgcccggcggcatgcagcagggcgcggggggctaccgcgaagcgcagagcctggtgcagggcgactacggcgctctgcagagcaacggacacccgctcagccacgctcaccagtggatcaccgcgctgtcccacggcggcggcggcgggggcggtggcggcggcggggggggcgggggcggcggcgggggcggcggcgacggctccccgtggtccaccagccccctgggccagccggacatcaagccctcggtggtggtgcagcagggcggccgcggagacgagctgcacgggccaggcgccctgcagcagcagcatcagcagcagcaacagcaacagcagcagcaacagcagcaacagcagcagcagcagcagcaacagcggccgccgcatctggtgcaccacgccgctaaccaccacccgggacccggggcatggcggagcgcggcggctgcagcgcacctcccaccctccatgggagcgtccaacggcggcttgctctactcgcagcccagcttcacggtgaacggcatgctgggcgccggcgggcagccggccggtctgcaccaccacggcctgcgggacgcgcacgacgagccacaccatgccgaccaccacccgcacccgcactcgcacccacaccagcagccgccgcccccgccgcccccgcagggtccgcctggccacccaggcgcgcaccacgacccgcactcggacgaggacacgccgacctcggacgacctggagcagttcgccaagcagttcaagcagcggcggatcaaactgggatttacccaagcggacgtggggctggctctgggcaccctgtatggcaacgtgttctcgcagaccaccatctgcaggtttgaggccctgcagctgagcttcaagaacatgtgcaagctgaagcctttgttgaacaagtggttggaggaggcggactcgtcctcgggcagccccacgagcatagacaagatcgcagcgcaagggcgcaagcggaaaaagcggacctccatcgaggtgagcgtcaagggggctctggagagccatttcctcaaatgccccaagccctcggcccaggagatcacctccctcgcggacagcttacagctggagaaggaggtggtgagagtttggttttgtaacaggagacagaaagagaaaaggatgacccctcccggagggactctgccgggcgccgaggatgtgtacggggggagtagggacactccaccacaccacggggtgcagacgcccgtccagtgaactcgagctgggggaggggcagagcgcggggctccccctccccttcggtccttggccctttcccggccctcttgttccctctctaacttctgattgttcttttatttttaattattatttccccgtcccttaaaaagacaaaaaaaataaggcaaaaggaaagcaactaagacactggactatcctttaaaggtagcaggtgtaatgatgtgttttgacctttgcaggcgagtaaccaggcaatggagtggagtgtctcctggagagagtgaggagagtgtgtgatagctagaaagagagagagacagagagatggcaagcactgagataaatacctggcaaaactaaataaattaccaaaaaggaaaaaaaatccaccaaaccatgataaacacaaaatgcagcttcctgatgcttagagttggcacatgctgctgtgtttatttattgtggattcccatcaggaaagaggaaaaaatacacatgttctttcatataggcaaaatttaaccacataaatttgcactgcaagaaaattgaagtttacgtgaacaaattcatgagcatattttctctttctccccaccgttaatttgggagttgccgttttgggggattttgttttgctttgctttattcatcggagagagttgaagccagctcttggccactctccatttctaatgttcttgtgttgccccttcttcgtactgtttgtgaactttggttaccttcacattccccttacgagggtgtaacatctatttgttcctcttaccaaagcaaaaggattggcttcatacaaaatagacaattctctgatttcaggaaatgtgcatggtctacccgctttatcgaaggcaagaatccggtttggaatataaaaataagcattggttgttcttaccagccacaaagtaaacttcattttcaggcagtgtttctgggggaggttatggagggaagaaaaaagaaaaatcgatagtgagtgactgattgcttcattttatcaggcgggcccattgtgaaagagctcaggggaaatgtggaggttaaatatatttccagagttgtccagcagaaagaaagtggcactttgaagagaactagggaagtacatatcttcagatatccctatatagttctctaccttcagttttagtaacaattatgaagaattatttgtgctgacagcagcagttaaactttgtttctctaatagctttttttttacataaaaaaagacccaggaacttaatagtgtatgcataagactgtgttttttagcacacagatacccacagcatacactgacgatctccacgcagtagacaggttttgtcttcactagctcatttgtttatcaagtcatatttagggtcccacaccctcttttcctgtaatttattgcagaatacaccactttgacttggacagctttctgccccctctttcactaaggaaggcaaatgaagtgaaaaaaaaaaatgccattttcaatccttcctttctcccctttgttaatagttttaagtgaatttttgaccttatcttaatggaaaacggttaactccaaacacaaaagactctactggaaagtgtaggtgaaaaaacttgtaactgtattgaaaataaataccattaaactgtgatcagttaaaatttaaaagaaaaatcagcacaaaagggcgctaaaagggaaaacactttttattaatcttaaaagtttgggggtttttttccagttaggtattagataaatttttattttaaaaaatgaaagtctcactaccataaaattatggttcagcatcagattagcattgcactcagtagtctttaaggttttaggaaatatgctttatattgtcttttcaaacacctgtgattgtttcattttccatgtttttgcaagataaatggtgacttataatgggcatatttatttgcctgtatttcatttcccccaatgaatgtcacaaggagatgggcacggagctgcttcgggtgcatcacgctgctcgttcctgaggtatgggaactggcctttagtgaagctatccagagcagggcaaatagccactggtaaagggaggaaatgaatttccagatacttattaccaagtaggtaaggtcagaagctggagttcagagaatgtgtctacagcttctctgactcttataggtttactaagatgaaagttaccactgaaccttaccactatgtatatatgtttaatatctgtcttttgaaatgcagaaatagtttaaatgtttctttgtctatttttctttttttttaatgctacccagggaaatattttcatatcatttttaagtggcctgcctcaatgtatatttatttcttttgaaacaaaaaggttctggaaactgtttttctgtagctttaaatgaataggtgagcaaaatctatatgggatgtaatttttttgttcagtctcttaaaaaatactttgttttggtacatttggttgtgcttgtggggaaaataaaaacgcagagatccttatatatttatgttaaagtaatattttattatctacataaaacagaaatgcacaataccttcatagtttgttctaattattgaaatatctttattttatttttaaagatagtgccaagttttaaggggggaaaaccctagaccttaaattgactgagttgagttgtgtgtaaaacacttcccttcctttatacttcataaagttttggaataaattttatgcatatactgccagatttgatgttcataactttcagaggctttttttttttttaatggagactactggtctaattcactttactttgcaaaaactatcagtcccaaatctttcagtcactatgcctgtagcattaaattgaaatggtcattgggtttgagcttcaatttgcttgccatttcatggtcctacaaagagatgtttgtcccctttaaacatatgcagatatgcctgcatcttctttccaggttaactataccttattcgattgtgctgttttggagaacatcagtggaactaggtggactttgatctctacccataggtcctcaaataatttgggatctacagaaaagcaggatttagtagattattatttttaataatttacaaatatctcttaacaaagaataaccctgatagtatactattgtgagtttactaaatgattagaagtagatcctatacattattcctgtttggtttgcataaaaagatgaatttta

SEQ ID NO: 51 - RNA, 6234 nt, Homo sapiens
  source 1..6234: mol_type = mRNA
cccttgcttttggtgatggttgtgagtgcagaagttgattgacagatgcatgccagaaacccccattctccttttcaaagacaacatatgatggattgctatctctcagcgcagcaggacacggggaccatgcaagctgtaattggtcaggcatgaactctcttgggttgtgttaccctgtttgctgtttactgtcaagatgctgaaagaatgttcttataatgatccaagaggaagtggcaaatgagtgctcttcttcaggatccaatgatagctggacaagtcagtaagcccttgctgtcagtgcggagtgaaatgaatgcggagttgagaggtgaggacaaggctgctacttcagacagcgagctgaatgagcccctgcttgcgcctgtggaatcaaatgacagcgaggacactcccagcaagctcttcggggctagaggaaacccagcattatcagacccaggcactcctgaccaacaccaggccagtcagacccaccccccatttccagttgggccacagccacttctgacggcacagcagttagcttctgctgtggccggcgtgatgccgggaggccccccagccctcaaccagccaatcctcattcccttcaacatggcgggacagctaggaggccagcaaggactggttctcacactgccaacagcgaatctcaccaacatccaagggctggtggcagcagctgcagccggaggcattatgactctgccactgcaaaatctacaagctacctcatccctgaactcccagctccagcagctccagctccagctccagcagcagcagcagcagcagcagcagcagcctcccccgtcaaccaaccagcacccgcaaccagccccacaggcgccctcgcagtcccagcagcagccgctgcagcccaccccaccccagcagccaccacccgcctctcagcagccgccagctcctacatctcagctgcaacaggcgcctcagccccagcagcaccaaccccactcccactcccagaaccagaaccaaccatctccaacccagcagagctccagccccccgcagaaacctagtcagtctccaggacatggcctgccttcaccgctcacgccacccaatcctctacagctggttaataatccactagcaagtcaggctgcagcggctgcagcagccatgagctccatagcaagctcacaggcctttggcaatgccctctccagtcttcagggggtcacaggtcaactagttactaatgcacaaggacagattatcgggaccattccactgatgcctaatccagggccatcgagccaagcagcaagcggcactcagggcttgcaagtgcagccaatcaccccccagctcctcacaaacgcccagggccagatcatcgccacagtcattgggaaccagatcctgcccgtgatcaacacccagggcatcacgctgtcacccatcaagcccggccagcagctccaccaaccctcccagacgtcagtgggtcaagcagcctcccaaggcaaccttctgcacctggctcacagccaagcatccatgtctcaaagtcccgtccggcaggcttcctcttcttcctcctcatcctcctcttcttcagctttgagcgtgggccagttagtcagcaatcctcaaacggcagcgggtgaggtggatggggttaatctggaggagatccgagaatttgccaaagcttttaaaatccggcgcctgtcccttggcctgacccagactcaggtgggacaggctctcagtgctacagagggccccgcgtacagccagtcggccatctgcagacacaccatcctgagaagccactttttcctaccacaggaagcccaagagaacactatagctagcagtctgacagccaaactgaaccctggccttttgtatcctgccaggtttgaaaagctggacatcacccctaaaagtgcccagaagatcaagccggtgcttgagcggtggatggctgaggctgaggcccgccatcgagcaggtatgcagaacctgaccgagtttatcgggagtgaaccatccaaaaagcgcaagcggcgcacctccttcacaccccaggcccttgagatcctcaatgcccactttgagaagaacacacacccttctgggcaggaaatgaccgaaattgctgagaagctgaactatgaccgagaagtagttagagtttggttctgcaataagaggcaagccctgaagaacacaattaaacgcttaaaacagcacgagccggccacggcagtccctttggagcccttaacagactctctggaagaaaactcctaaagagatgcccacccataatcagaagcaaaattcacagaaactaaactccacccttgggactccacaacaacaacaacaacaaaatttaatttaatttaaaaatagccccagtcgtcatcacccttgtaagtaaatgactaagaaaactaccaagtggacagaatggtttctacatgtccgttggttttccaaaaaggaaagaagaaaatttttagaaaatttttaaacaaggaatacaccacactgaaggtgtgtgtggtaggatagttcccttcccccacctgtctcccccaaagccagttttttaatggacttaaagcaaaccaaataaccacgtacttttttctgtatattatgaaaatgtgaacacattttaaggaaaaagaaaaaaaaaaactaaaccaaaaaccaacaacgaaggaacaaaaactttgatctgttcaaagcgaatacaagcctgccacctggaggaaggactgcaccccttcaggtactaagtgctgattcactatgaaacctattaaccaaagtcagaaacatggcattgcaacgcgatcgttcgtctacgcttctccgcacgtaaagttgtgttacgaatttttacatttgtactaacagaacaataggaagcctgatttctcccatctttcccatctactgtctccacccatggggtgggtaccattgttgaagccattctgtgaggctcactattgggtttttttgtgggggtggtagggagggtggtcttttttctttgtctttctttttgacgggagggcatcctggatcgtgtgccaaagcatttgttgcttttttctcactatgacttgtgggtttgagaaaagaaaatggagctcgcatttctctcttttcctccatttctccatctccctcgcgcgtggctcctggggtctgctggaaggccacagaagtggggagaagcagtgtcttttccactcagatcctagtgaaatgcaggagagactccaaaataactagggctttgctcgatgaactgtcaacactggcataagctgtaattgtgctcactgtccacaccagagcttgggatttttctcagtctgttggccacgtacatggagagctgaccaaaactaattttgtaatataaacataagctgcacatttggttcaatacttacatctatgttatgcttctgtgcaaagcaatttctctcagaagtctgatagccaaagaaatgtctcaagatttcagtcaaatacacacatgacatgcacacacccataaacacacacacaaagaaacaggctaaaaagaaagtgatagcaactggatcattaaaggccatcgtgtatcctgttaatctcatcttttctgctacttctttcattccaatagcgatggcagacttttttcagggggaataattactcttcgggatatataaatggagcagaaaggtggcgggcaaatttaaacaactggaattgggggccaggttcttcaggggaaaggaagtttgaagaagctttcaccaaaagaaaaaaatatagaaggggcttatctaacaatcagaatagcctgcaatatgagaactagaggattatttcctttaaaaaaaataacttaaaagatctgtgggccacaataacctaataagcaaggacatgttgggatgtgaggacggctgtgcaggtgagcacttggcagctgccactggtgttttcacccaggtacaggcagacggtgttccattaaaggagcaggcaaaagagagctgtcaatttgcttgctgttgaaatgtacatatttatgcataatttataatcatgctaatgtattatctagaagtaagttgtgaaagaaaaaagagaaacccaatctcaagttgcccaactgattagaaatgccatctccttcccctaatacactccctctccctggggagtttgagtttcttctgttggctcctttgctgtttgtgtaaatgctacagtataattgtcccctccaatgattcatgcagcaatcaagagactaccagcaaggaaactctaaaaggaatcctggaagtggaacaagtcactcgtaaaggtgtttccattttaccagtcaatgctaactggaccacagctaacttgatcttcaatgccaacatcacagagcccctttcgccacctcaaaaactgcacaaacatggacaacccagaaacccaaaactttattgattaaaacagattttggattctcctaggacctgctccaaattccatcaagaaaagctccctgcgaagagaagttaacaagataacatatgatggatgctaaatatttaaatatatgccggcagcggttacataaggcctgctcagttctgagatctataattgggagggtctaactaggacagggaaaatataaaataaaagtaatcttccagagtggaacaaaaaagaaatatattcatccgacagtcgtaacaccattcatgtgcagtgatttttttatagttttataattttgtattgttttataaattatttataaggtgttgtaatgcttcttatattaattttttacagataaattttttgctacaaggcataaaaaggtgcctgaaccgattcttaggaatataaattatactgtgtaactcataagtctttgggaccacacctaatgcttaattttatgatgacatttctcatgtctacttgtaaacaacttcatatgccaatggcgtttaagtgtcttttatgtttccatgaactgaaggttaaggttgccacccacaaaaaataaaagccactttgggcatatgtgattgtatttcaagcttcagtatttttctcacataatttttaattattgttatctgcattttcattacttcatgaaaaaatgacagtggatattacattttacacttgtttatagattttctttctttttcaaccaagaaaatattttgaacaattgaaacttttaattgcatttgcaaggttttggcttctttatgttgtcatcacacagatgcacacacacatacacatacaacacaaaagagagaacaaaaaagcaaaaggagctaggaaaaaaacaaaacaaaacaagtagaggtgtatcaaagaactcaagctataaccaaaaagaaatcgtaaaatgcctttgctcgtttctctacgctggaccaaagctcaatatttgtaggtatatgcacattgtatagatatggctaaatgttgctgacaatctcgcaatactaaactgttcctattttaagaaaaaaaaaagaaatacaaactgttcatcaatgttttacctcagcactctacttgtacccagttaatgaccaagcttaaaaaaaaattgagataaaggaaattgattttcatttcaatgtttgactgtaaaatctgtttggataacattttgtaatgagctttttgtcatgtgatttgcttgtcttcaacttgaaattatgtgaggcacatttgtttatttgttgttaagaaagtgatttttttttttttttgtcctatgtgctgtgatctagactggtcaccggggtcacttatgaggcaccacaaagagatctgcttctccgtgcccggagcaggcagcaggagggagggcaggaaacaggactgggttgctttggaaaaatcatcatgacaagaagttgacatgatagacttgtgaccaagaagccaaactgaatatttaaaagctccttacttgctctgacattgaaaccaaagctgattttatctgcacaggttgcttaatatttaaaaaaaaaaaaaactgtacttaatctagagcaatatctgtatggtcagtaaagctgcactttgtgtatttcttaacagcttcagatctgtcacttttaatttgtaccataaaaaataaataattgtttgacatga

SEQ ID NO: 52 - RNA, 2885 nt, Homo sapiens
  source 1..2885: mol_type = mRNA
agtcgctcagtcagtctcgggctgtccggccagggtggttggtggtaaggattcaggctccgtcctaacgaggccgtggcctgaggctcagggccccccgcccctccctcccagcccaccagcgtcacctcccagccccgagctggaccgcacaccttgggacacggttttccacttcctaaggacgagccccagactggaggagaggtccgaggaggtgggcgttggactctttgcgaggaccccggcggctggcccgggggaggcggccgaggcggcggcggcggcggccgggggcgacatggcggaggagcaggacctatcggaggtggagctgagccccgtgggctcggaggagccccgctgcctgtccccggggagcgcgccctcgctagggcccgacggcggcggcggcggatcgggcctgcgagccagcccggggccaggcgagctgggcaaggtcaagaaggagcagcaggacggcgaggcggacgatgacaagttccccgtgtgcatccgcgaggccgtcagccaggtgctcagcggctacgactggacgctggtgcccatgcccgtgcgcgtcaacggcgccagcaaaagcaagccgcacgtcaagcggcccatgaacgccttcatggtgtgggctcaggcagcgcgcaggaagctcgcggaccagtacccgcacctgcacaacgctgagctcagcaagacgctgggcaagctctggaggctgctgaacgaaagtgacaagcgccccttcatcgaggaggctgagcggctccgtatgcagcacaagaaagaccacccggactacaagtaccagcccaggcggcggaagaacgggaaggccgcccagggcgaggcggagtgccccggtggggaggccgagcaaggtgggaccgccgccatccaggcccactacaagagcgcccacttggaccaccggcacccaggagagggctcccccatgtcagatgggaaccccgagcacccctcaggccagagccatggcccacccacccctccaaccaccccgaagacagagctgcagtcgggcaaggcagacccgaagcgggacgggcgctccatgggggagggcgggaagcctcacatcgacttcggcaacgtggacattggtgagatcagccacgaggtaatgtccaacatggagacctttgatgtggctgagttggaccagtacctgccgcccaatgggcacccaggccatgtgagcagctactcagcagccggctatgggctgggcagtgccctggccgtggccagtggacactccgcctggatctccaagccaccaggcgtggctctgcccacggtctcaccacctggtgtggatgccaaagcccaggtgaagacagagaccgcggggccccaggggcccccacactacaccgaccagccatccacctcacagatcgcctacacctccctcagcctgccccactatggctcagccttcccctccatctcccgcccccagtttgactactctgaccatcagccctcaggaccctattatggccactcgggccaggcctctggcctctactcggccttctcctatatggggccctcgcagcggcccctctacacggccatctctgaccccagcccctcagggccccagtcccacagccccacacactgggagcagccagtatatacgacactgtcccggccctaaagggggccctgtcgccaccaccccccgcccagcccctgcccccagcctgtgtgccctgttccttgcccacctcaggcctggtggtggcagtggaggaggctgaggaggctgaagaggctgacaggtcggggggctttctgtctggctcactgccctgatgacccacccgccccatccaggctccagcagcaaagccccaggagaacaggctggacagaggagaaggaggttgactgttgcacccacactgaaagatgaggggctgcaccttcccccaggaatgaccctctatcccaggacctgagaagggcctgctcaccctcctcggggaggggaagcaccagggttggtggcatcggaggccttaccactcctatgactcctgttttctctctcacagatagtgagggtctgacatgcccatgccacctatgccacagtgcctaagggctaggccacccagagactgtgcccggagctggccgtgtctcccactcaggggctgagagtagctttgaggagcctcattggggagtggggggttcgagggacttagtggagttctcatcccttcaatgccccctccctttctgaaggcaggaaggagttggcacagaggccccctgatccaattctgtgccaataacctcattctttgtctgagaaacagcccccagtcctcctccactacaacctccatgaccttgagacgcatcccaggaggtgacgaggcaggggctccaggaaaggaatcagagacaattcacagagcctccctccctgggctccttgccagctccctcttcccttactaggctctatggcccctgctcagtcagccccactccctgggcttcccagagagtgacagctgctcaggccctaacccttggctccaggagacacagggcccagcacccaggttgctgtcggcaggctgaagacactagaatcctgacctgtacattctgcccttgcctcttaccccttgcctcccagtggtatttgaataaagtatgtagctatatctgcccctattttcctgttctgcagccccccaaatccacatgtaactcattactgtctcctgttatttatctcagtagtcccctctcctagccactctagcccctattaactctgcattaagcattccacataataaaattaaaggttccggtta

SEQ ID NO: 53 - protein, 432 aa, Homo sapiens
MERRRITSAARRSYVSSGEMMVGGLAPGRRLGPGTRLSLARMPPPLPTRVDFSLAGALNAGFKETRASERAEMMELNDRFASYIEKVRFLEQQNKALAAELNQLRAKEPTKLADVYQAELRELRLRLDQLTANSARLEVERDNLAQDLATVRQKLQDETNLRLEAENNLAAYRQEADEATLARLDLERKIESLEEEIRFLRKIHEEEVRELQEQLARQQVHVELDVAKPDLTAALKEIRTQYEAMASSNMHEAEEWYRSKFADLTDAAARNAELLRQAKHEANDYRRQLQSLTCDLESLRGTNESLERQMREQEERHVREAASYQEALARLEEEGQSLKDEMARHLQEYQDLLNVKLALDIEIATYRKLLEGEENRITIPVQTFSNLQIRETSLDTKSVSEGHLKRNIVVKTVEMRDGEVIKESKQEHKDVM

SEQ ID NO: 54 - protein, 430 aa, Rattus norvegicus
MERRRITSARRSYASSETMVRGHGPTRHLGTIPRLSLSRMTPPLPARVDFSLAGALNAGFKETRASERAEMMELNDRFASYIEKVRFLEQQNKALAAELNQLRAKEPTKLADVYQAELRELRLRLDQLTTNSARLEVERDNLTQDLGTLRQKLQDETNLRLEAENNLAVYRQEADEATLARVDLERKVESLEEEIQFLRKIHEEEVRELQEQLAQQQVHVEMDVAKPDLTAALREIRTQYEAVATSNMQETEEWYRSKFADLTDVASRNAELLRQAKHEANDYRRQLQALTCDLESLRGTNESLERQMREQEERHARESASYQEALARLEEEGQSLKEEMARHLQEYQDLLNVKLALDIEIATYRKLLEGEENRITIPVQTFSNLQIRETSLDTKSVSEGHLKRNIVVKTVEMRDGEVIKESKQEHKDVM

SEQ ID NO: 55 - protein, 430 aa, Mus musculus
MERRRITSARRSYASETVVRGLGPSRQLGTMPRFSLSRMTPPLPARVDFSLAGALNAGFKETRASERAEMMELNDRFASYIEKVRFLEQQNKALAAELNQLRAKEPTKLADVYQAELRELRLRLDQLTANSARLEVERDNFAQDLGTLRQKLQDETNLRLEAENNLAAYRQEADEATLARVDLERKVESLEEEIQFLRKIYEEEVRELREQLAQQQVHVEMDVAKPDLTAALREIRTQYEAVATSNMQETEEWYRSKFADLTDAASRNAELLRQAKHEANDYRRQLQALTCDLESLRGTNESLERQMREQEERHARESASYQEALARLEEEGQSLKEEMARHLQEYQDLLNVKLALDIEIATYRKLLEGEENRITIPVQTFSNLQIRETSLDTKSVSEGHLKRNIVVKTVEMRDGEVIKDSKQEHKDVVM

SEQ ID NO: 56 - protein, 466 aa, Homo sapiens; identical to SEQ ID NO: 17

SEQ ID NO: 57 - protein, 466 aa, Rattus norvegicus
MAEEQDLSEVELSPVGSEEPRCLSPSSAPSLGPDGGGGGSGLRASPGPGELGKVKKEQQDGEADDDKFPVCIREAVSQVLSGYDWTLVPMPVRVNGASKSKPHVKRPMNAFMVWAQAARRKLADQYPHLHNAELSKTLGKLWRLLNESDKRPFIEEAERLRMQHKKDHPDYKYQPRRRKNGKAAQGEAECPGGETDQGGAAAIQAHYKSAHLDHRHPEEGSPMSDGNPEHPSGQSHGPPTPPTTPKTELQSGKADPKRDGRSLGEGGKPHIDFGNVDIGEISHEVMSNMETFDVTELDQYLPPNGHPGHVGSYSAAGYGLSSALAVASGHSAWISKPPGVALPTVSPPAVDAKAQVKTETTGPQGPPHYTDQPSTSQIAYTSLSLPHYGSAFPSISRPQFDYSDHQPSGPYYGHAGQASGLYSAFSYMGPSQRPLYTAISDPSPSGPQSHSPTHWEQPVYTTLSRP

SEQ ID NO: 58 - protein, 466 aa, Mus musculus
MAEEQDLSEVELSPVGSEEPRCLSPGSAPSLGPDGGGGGSGLRASPGPGELGKVKKEQQDGEADDDKFPVCIREAVSQVLSGYDWTLVPMPVRVNGASKSKPHVKRPMNAFMVWAQAARRKLADQYPHLHNAELSKTLGKLWRLLNESDKRPFIEEAERLRMQHKKDHPDYKYQPRRRKNGKAAQGEAECPGGEAEQGGAAAIQAHYKSAHLDHRHPEEGSPMSDGNPEHPSGQSHGPPTPPTTPKTELQSGKADPKRDGRSLGEGGKPHIDFGNVDIGEISHEVMSNMETFDVTELDQYLPPNGHPGHVGSYSAAGYGLGSALAVASGHSAWISKPPGVALPTVSPPGVDAKAQVKTETTGPQGPPHYTDQPSTSQIAYTSLSLPHYGSAFPSISRPQFDYSDHQPSGPYYGHAGQASGLYSAFSYMGPSQRPLYTAISDPSPSGPQSHSPTHWEQPVYTTLSRP

SEQ ID NO: 59 - protein, 408 aa, Homo sapiens
MIPGNRMLMVVLLCQVLLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEVISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 60 - protein, 288 aa, Homo sapiens
MVGVGGGDVEDVTPRPGGCQISGRGARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 61 - protein, 296 aa, Homo sapiens
MEIYSPDMSEVAAERSSSPSTQLSADPSLDGLPAAEDMPEPQTEDGRTPGLVGLAVPCCACLEAERLRGCLNSEKICIVPILACLVSLCLCIAGLKWVFVDKIFEYDSPTHLDPGGLGQDPIISLDATAASAVWVSSEAYTSPVSRAQSESEVQVTVQGDKAVVSFEPSAAPTPKNRIFAFSFLPSTAPSFPSPTRNPEVRTPKSATQPQTTETNLQTAPKLSTSTSTTGTSHLVKCAEKEKTFCVNGGECFMVKDLSNPSRYLCKCPNEFTGDRCQNYVMASFYSTSTPFLSLPE